(19) United States
(12) Patent Application Publication
(10) Pub. No.: US 2024/0199587 A1
(43) Pub. Date: Jun. 20, 2024

(54) HETEROCYCLIC COMPOUNDS

(71) Applicant: Hoffmann-La Roche Inc., Little Falls, NJ (US)

(72) Inventors: Machoud AMOUSSA, Berlin (DE); Joerg BENZ, Rheinfelden (DE); Niels Kevin BRIAN, Weil am Rhein (DE); Kallie FRISTON, Stockton-On-Tees (GB); Maude GIROUD, Basel (CH); Uwe GRETHER, Efringen-Kirchen (DE); Katrin GROEBKE ZBINDEN, Liestal (CH); Benoit HORNSPERGER, Altkirch (FR); Carsten KROLL, Basel (CH); Bernd KUHN, Reinach BL (CH); Camiel John LEAKE, Brasschaat (BE); Rainer E. MARTIN, Basel (CH); David Friedrich Erhard NIPPA, Muehldorf am Inn (DE); Fionn Susannah O’HARA, Basel (CH); Bernd PUELLMANN, Aesch (CH); Hans RICHTER, Grenzach-Wyhlen (DE); Martin RITTER, Mumpf (CH); Didier ROMBACH, Mulhouse (FR); Philipp Claudio SCHMID, Birsfelden (CH); Shounan ZHANG, Hubei (CN)

(73) Assignee: Hoffmann-La Roche Inc., Little Falls, NJ (US)

(21) Appl. No.: 18/490,967

(22) Filed: Oct. 20, 2023

Related U.S. Application Data
(63) Continuation of application No. PCT/EP2022/060644, filed on Apr. 22, 2022.

(30) Foreign Application Priority Data
Apr. 23, 2021 (EP) 21170090.1
Mar. 25, 2022 (WO) PCT/CN2022/083125

(51) Int. Cl. C07D 403/14; A61K 31/4178; A61K 31/4196; A61K 31/4245; A61K 31/427; A61K 31/438; A61K 31/4439; A61K 31/496; A61K 31/497; A61K 31/506; A61K 31/5377; C07D 401/14; C07D 407/14; C07D 413/14; C07D 417/14; C07D 471/10; C07D 487/10; C07D 491/052

(52) U.S. Cl. CPC C07D 403/14; A61K 31/4178; A61K 31/4196; A61K 31/4245; A61K 31/427; A61K 31/438; A61K 31/4439; A61K 31/496; A61K 31/497; A61K 31/506; A61K 31/5377; C07D 401/14; C07D 407/14; C07D 413/14; C07D 417/14; C07D 471/10; C07D 487/10; C07D 491/052

(57) ABSTRACT

The invention provides new heterocyclic compounds having the general formula (I)

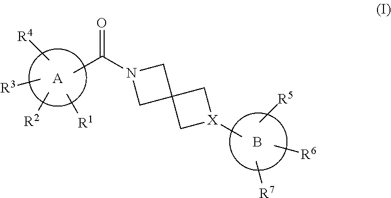

wherein A, B, X, and R¹ to R⁷ are as described herein, compositions including the compounds, processes of manufacturing the compounds and methods of using the compounds.

### CROSS REFERENCE TO RELATED APPLICATIONS

This application is a continuation of International Application No. PCT/EP2022/060644, filed Apr. 22, 2022, which claims priority to Chinese Application No. PCT/CN2022/083125, filed Mar. 25, 2022, and EP Application No. 21170090.1, filed Apr. 23, 2021, the disclosure of each of which is incorporated herein by reference in its entirety.

### FIELD OF THE INVENTION

The present invention relates to organic compounds useful for therapy or prophylaxis in a mammal, and in particular to monoacylglycerol lipase (MAGL) inhibitors for the treatment or prophylaxis of neuroinflammation, neurodegenerative diseases, pain, cancer, mental disorders, multiple sclerosis, Alzheimer's disease, Parkinson's disease, amyotrophic lateral sclerosis, traumatic brain injury, neurotoxicity, stroke, epilepsy, anxiety, migraine, depression, inflammatory bowel disease, abdominal pain, abdominal pain associated with irritable bowel syndrome and/or visceral pain in a mammal.

### BACKGROUND OF THE INVENTION

Endocannabinoids (ECs) are signaling lipids that exert their biological actions by interacting with cannabinoid receptors (CBRs), CB1 and CB2. They modulate multiple physiological processes including neuroinflammation, neurodegeneration and tissue regeneration (Iannotti, F. A., et al., Progress in lipid research 2016, 62, 107-28). In the brain, the main endocannabinoid, 2-arachidonoylglycerol (2-AG), is produced by diacyglycerol lipases (DAGL) and hydrolyzed by the monoacylglycerol lipase, MAGL. MAGL hydrolyses 85% of 2-AG; the remaining 15% being hydrolysed by ABHD6 and ABDH12 (Nomura, D. K., et al., Science 2011, 334, 809). MAGL is expressed throughout the brain and in most brain cell types, including neurons, astrocytes, oligodendrocytes and microglia cells (Chanda, P. K., et al., Molecular pharmacology 2010, 78, 996; Viader, A., et al., Cell reports 2015, 12, 798). 2-AG hydrolysis results in the formation of arachidonic acid (AA), the precursor of prostaglandins (PGs) and leukotrienes (LTs). Oxidative metabolism of AA is increased in inflamed tissues. There are two principal enzyme pathways of arachidonic acid oxygenation involved in inflammatory processes, the cyclo-oxygenase which produces PGs and the 5-lipoxygenase which produces LTs. Of the various cyclooxygenase products formed during inflammation, PGE2 is one of the most important. These products have been detected at sites of inflammation, e.g. in the cerebrospinal fluid of patients suffering from neurodegenerative disorders and are believed to contribute to inflammatory response and disease progression. Mice lacking MAGL (Mgl1−/−) exhibit dramatically reduced 2-AG hydrolase activity and elevated 2-AG levels in the nervous system while other arachidonoyl-containing phospho- and neutral lipid species including anandamide (AEA), as well as other free fatty acids, are unaltered. Conversely, levels of AA and AA-derived prostaglandins and other eicosanoids, including prostaglandin E2 (PGE2), D2 (PGD2), F2 (PGF2), and thromboxane B2 (TXB2), are strongly decreased. Phospholipase A₂ (PLA₂) enzymes have been viewed as the principal source of AA, but cPLA2-deficient mice have unaltered AA levels in their brain, reinforcing the key role of MAGL in the brain for AA production and regulation of the brain inflammatory process.

Neuroinflammation is a common pathological change characteristic of diseases of the brain including, but not restricted to, neurodegenerative diseases (e.g. multiple sclerosis, Alzheimer's disease, Parkinson disease, amyotrophic lateral sclerosis, traumatic brain injury, neurotoxicity, stroke, epilepsy and mental disorders such as anxiety and migraine). In the brain, production of eicosanoids and prostaglandins controls the neuroinflammation process. The pro-inflammatory agent lipopolysaccharide (LPS) produces a robust, time-dependent increase in brain eicosanoids that is markedly blunted in Mgll−/− mice. LPS treatment also induces a widespread elevation in pro-inflammatory cytokines including interleukin-1-a (IL-1-a), IL-1b, IL-6, and tumor necrosis factor-a (TNF-α) that is prevented in Mgll−/− mice.

Neuroinflammation is characterized by the activation of the innate immune cells of the central nervous system, the microglia and the astrocytes. It has been reported that anti-inflammatory drugs can suppress in preclinical models the activation of glia cells and the progression of disease including Alzheimer's disease and mutiple sclerosis (Lleo A., Cell Mol Life Sci. 2007, 64, 1403). Importantly, genetic and/or pharmacological disruption of MAGL activity also blocks LPS-induced activation of microglial cells in the brain (Nomura, D. K., et al., Science 2011, 334, 809).

In addition, genetic and/or pharmacological disruption of MAGL activity was shown to be protective in several animal models of neurodegeneration including, but not restricted to, Alzheimer's disease, Parkinson's disease and multiple sclerosis. For example, an irreversible MAGL inhibitor has been widely used in preclinical models of neuroinflammation and neurodegeneration (Long, J. Z., et al., Nature chemical biology 2009, 5, 37). Systemic injection of such inhibitor recapitulates the Mgll−/− mice phenotype in the brain, including an increase in 2-AG levels, a reduction in AA levels and related eicosanoids production, as well as the prevention of cytokines production and microglia activation following LPS-induced neuroinflammation (Nomura, D. K., et al., Science 2011, 334, 809), altogether confirming that MAGL is a druggable target.

Consecutive to the genetic and/or pharmacological disruption of MAGL activity, the endogenous levels of the MAGL natural substrate in the brain, 2-AG, are increased. 2-AG has been reported to show beneficial effects on pain with, for example, anti-nociceptive effects in mice (Ignatowska-Jankowska B. et al., J. Pharmacol. Exp. Ther. 2015, 353, 424) and on mental disorders, such as depression in chronic stress models (Zhong P. et al., Neuropsychopharmacology 2014, 39, 1763).

Furthermore, oligodendrocytes (OLs), the myelinating cells of the central nervous system, and their precursors (OPCs) express the cannabinoid receptor 2 (CB2) on their membrane. 2-AG is the endogenous ligand of CB1 and CB2 receptors. It has been reported that both cannabinoids and pharmacological inhibition of MAGL attenuate OLs's and OPCs's vulnerability to excitotoxic insults and therefore may be neuroprotective (Bernal-Chico, A., et al., Glia 2015, 63, 163). Additionally, pharmacological inhibition of MAGL increases the number of myelinating OLs in the brain of mice, suggesting that MAGL inhibition may promote differentiation of OPCs in myelinating OLs in vivo (Alpar, A., et al., Nature communications 2014, 5, 4421). Inhibition of MAGL was also shown to promote remyelination and functional recovery in a mouse model of progressive multiple sclerosis (Feliu A. et al., Journal of Neuroscience 2017, 37 (35), 8385).

In addition, in recent years, metabolism is talked highly important in cancer research, especially the lipid metabolism. Researchers believe that the de novo fatty acid synthesis plays an important role in tumor development. Many studies illustrated that endocannabinoids have anti-tumorigenic actions, including anti-proliferation, apoptosis induction and anti-metastatic effects. MAGL as an important decomposing enzyme for both lipid metabolism and the endocannabinoids system, additionally as a part of a gene expression signature, contributes to different aspects of tumourigenesis, including in glioblastoma (Qin, H., et al., Cell Biochem. Biophys. 2014, 70, 33; Nomura D K et al., Cell 2009, 140(1), 49-61; Nomura D K et al., Chem. Biol. 2011, 18(7), 846-856, Jinlong Yin et al, Nature Communications 2020, 11, 2978).

The endocannabinoid system is also involved in many gastrointestinal physiological and physiopathological actions (Marquez, Suarez et al. 2009). All these effects are driven mainly via cannabinoid receptors (CBRs), CB1 and CB2. CB1 receptors are present throughout the GI tract of animals and healthy humans, especially in the enteric nervous system (ENS) and the epithelial lining, as well as smooth muscle cells of blood vessels in the colonic wall (Wright, Rooney et al. 2005), (Duncan, Davison et al. 2005). Activation of CB1 produces anti-emetic, anti-motility, and anti-inflammatory effect, and help to modulate pain (Perisetti, Rimu et al. 2020). CB2 receptors are expressed in immune cells such as plasma cells and macrophages, in the lamina propria of the GI tract (Wright, Rooney et al. 2005), and primarily on the epithelium of human colonic tissue associated with inflammatory bowel disease (IBD). Activation of CB2 exerts anti-inflammatory effect by reducing pro-inflammatory cytokines. Expression of MAGL is increased in colonic tissue in UC patients (Marquez, Suarez et al. 2009) and 2-AG levels are increased in plasma of IBD patients (Grill, Hogenauer et al. 2019). Several animal studies have demonstrated the potential of MAGL inhibitors for symptomatic treatment of IBD. MAGL inhibition prevents TNBS-induced mouse colitis and decreases local and circulating inflammatory markers via a CB1/CB2 MoA (Marquez, Suarez et al. 2009). Furthermore, MAGL inhibition improves gut wall integrity and intestinal permeability via a CB1 driven MoA (Wang, Zhang et al. 2020).

In conclusion, suppressing the action and/or the activation of MAGL is a promising new therapeutic strategy for the treatment or prevention of neuroinflammation, neurodegenerative diseases, pain, cancer, mental disorders, inflammatory bowel disease, abdominal pain and abdominal pain associated with irritable bowel syndrome. Furthermore, suppressing the action and/or the activation of MAGL is a promising new therapeutic strategy for providing neuroprotection and myelin regeneration. Accordingly, there is a high unmet medical need for new MAGL inhibitors.

### SUMMARY OF THE INVENTION

In a first aspect, the present invention provides compounds of formula (I)

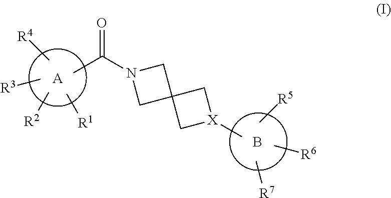

wherein A, B, X, and R¹ to R⁷ are as defined herein.

In a further aspect, the present invention provides a process of manufacturing the compounds of formula (I) described herein, or pharmaceutically acceptable salts thereof, wherein the process is as described in any one of schemes 1 to 44.

In a further aspect, the present invention provides a compound of formula (I) as described herein, or a pharmaceutically acceptable salt thereof, when manufactured according to the processes described herein.

In a further aspect, the present invention provides a compound of formula (I) as described herein, or a pharmaceutically acceptable salt thereof, for use as therapeutically active substance.

In a further aspect, the present invention provides a pharmaceutical composition comprising a compound of formula (I) as described herein, or a pharmaceutically acceptable salt thereof, and a therapeutically inert carrier.

In a further aspect, the present invention provides a compound of formula (I) as described herein, or a pharmaceutically acceptable salt thereof, for use in a method of inhibiting monoacylglycerol lipase in a mammal.

In a further aspect, the present invention provides a compound of formula (I) as described herein, or a pharmaceutically acceptable salt thereof, for use in the treatment or prophylaxis of neuroinflammation, neurodegenerative diseases, pain, cancer, mental disorders and/or inflammatory bowel disease in a mammal.

In a further aspect, the present invention provides a compound of formula (I) as described herein, or a pharmaceutically acceptable salt thereof, for use in the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “alkyl” refers to a mono- or multivalent, e.g., a mono- or bivalent, linear or branched saturated hydrocarbon group of 1 to 12 carbon atoms. In some preferred embodiments, the alkyl group contains 1 to 6 carbon atoms (“C₁₋₆-alkyl”), e.g., 1, 2, 3, 4, 5, or 6 carbon atoms. In other embodiments, the alkyl group contains 1 to 3 carbon atoms, e.g., 1, 2 or 3 carbon atoms. Some non-limiting examples of alkyl include methyl, ethyl, propyl, 2-propyl (isopropyl), n-butyl, iso-butyl, sec-butyl, tert-butyl, and 2,2-dimethylpropyl. Particularly preferred, yet non-limiting examples of alkyl are methyl, tert-butyl, and 2,2-dimethylpropyl.

The term “alkoxy” refers to an alkyl group, as previously defined, attached to the parent molecular moiety via an oxygen atom. Unless otherwise specified, the alkoxy group contains 1 to 12 carbon atoms. In some preferred embodiments, the alkoxy group contains 1 to 6 carbon atoms (“C₁₋₆-alkoxy”). In other embodiments, the alkoxy group contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy. A particularly preferred, yet non-limiting example of alkoxy is methoxy.

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “cycloalkyl” as used herein refers to a saturated or partly unsaturated monocyclic or bicyclic hydrocarbon group of 3 to 10 ring carbon atoms (“C₃₋₁₀-cycloalkyl”). In some preferred embodiments, the cycloalkyl group is a saturated monocyclic hydrocarbon group of 3 to 8 ring carbon atoms. “Bicyclic cycloalkyl” refers to cycloalkyl moieties consisting of two saturated carbocycles having two carbon atoms in common, i.e., the bridge separating the two rings is either a single bond or a chain of one or two ring atoms, and to spirocyclic moieties, i.e., the two rings are connected via one common ring atom. Preferably, the cycloalkyl group is a saturated monocyclic hydrocarbon group of 3 to 6 ring carbon atoms, e.g., of 3, 4, 5 or 6 carbon atoms. Some non-limiting examples of cycloalkyl include cyclopropyl, cyclobutyl, cyclopentyl, cyclohexyl, cycloheptyl, 1-bicyclo[1.1.1]pentanyl, norbornanyl, and 1-bicyclo[2.2.2]octanyl. A particularly preferred, yet non-limiting example of cycloalkyl is cyclopropyl.

The term “aryl” refers to a monocyclic, bicyclic, or tricyclic carbocyclic ring system having a total of 6 to 14 ring members (“C₆-C₁₄-aryl”), preferably, 6 to 12 ring members, and more preferably 6 to 10 ring members, and wherein at least one ring in the system is aromatic. Some non-limiting examples of aryl include phenyl and 9H-fluorenyl (e.g. 9H-fluoren-9-yl). A particularly preferred, yet non-limiting example of aryl is phenyl.

The term “haloaryl” refers to an aryl group, wherein at least one of the hydrogen atoms of the aryl group has been replaced by a halogen atom, preferably fluoro. Preferably, “haloaryl” refers to an aryl group wherein 1, 2 or 3 hydrogen atoms of the aryl group have been replaced by a halogen atom, most preferably fluoro. A particularly preferred, yet non-limiting examples of haloaryl is fluorophenyl.

The term “heteroaryl” refers to a mono- or multivalent, monocyclic, bicyclic or tricyclic, preferably bicyclic ring system having a total of 5 to 14 ring members, preferably, 5 to 12 ring members, and more preferably 5 to 10 ring members, wherein at least one ring in the system is aromatic, and at least one ring in the system contains one or more heteroatoms. Preferably, “heteroaryl” refers to a 5-10 membered heteroaryl comprising 1, 2, 3 or 4 heteroatoms independently selected from O, S and N. Most preferably, “heteroaryl” refers to a 5-10 membered heteroaryl comprising 1 to 2 heteroatoms independently selected from O, S and N. Some non-limiting examples of heteroaryl include spiro[cyclopropane-1,3′-indoline] (e.g., spiro[cyclopropane-1,3′-indoline]-1′-yl), 2-pyridyl, 3-pyridyl, 4-pyridyl, pyrazin-2-yl, pyrimidin-2-yl, pyrimidin-4-yl, pyrimidin-5-yl, pyrimidin-6-yl, indol-1-yl, 1H-indol-2-yl, 1H-indol-3-yl, 1H-indol-4-yl, 1H-indol-5-yl, 1H-indol-6-yl, 1H-indol-7-yl, 1,2-benzoxazol-3-yl, 1,2-benzoxazol-4-yl, 1,2-benzoxazol-5-yl, 1,2-benzoxazol-6-yl, 1,2-benzoxazol-7-yl, 1H-indazol-3-yl, 1H-indazol-4-yl, 1H-indazol-5-yl, 1H-indazol-6-yl, 1H-indazol-7-yl, pyrazol-1-yl, 1H-pyrazol-3-yl, 1H-pyrazol-4-yl, 1H-pyrazol-5-yl, imidazol-1-yl, 1H-imidazol-2-yl, 1H-imidazol-4-yl, 1H-imidazol-5-yl, oxazol-2-yl, oxazol-4-yl, oxazol-5-yl, 1,2,4-oxadiazolyl, 1,3,4-oxadiazolyl, thiazol-2-yl, thiazol-4-yl, thiazol-5-yl, pyridazin-3-yl, pyridazin-4-yl, 1,2,4-triazol-4-yl, 1,2,4-triazol-1-yl, 4H-1,2,4-triazol-3-yl, 4,5,6,7-tetrahydroindazol-2-yl, 6,7-dihydro-4H-pyrano[4,3-c]pyrazol-2-yl, thiazolyl, benzofurazan-4-yl, tetrazolyl, isoxazolyl, and morpholinyl. Particularly preferred, yet non-limiting examples of heteroaryl are pyridyl, pyrazinyl, pyrimidinyl, pyrazolyl, imidazolyl, oxazolyl, oxadiazolyl and triazolyl.

The term “heterocyclyl” refers to a saturated or partly unsaturated mono- or bicyclic, preferably monocyclic ring system of 3 to 14 ring atoms, preferably 3 to 10 ring atoms, more preferably 3 to 8 ring atoms, wherein 1, 2, or 3 of said ring atoms are heteroatoms selected from N, O and S, the remaining ring atoms being carbon. Preferably, 1 to 2 of said ring atoms are selected from N and O, the remaining ring atoms being carbon. “Bicyclic heterocyclyl” refers to heterocyclic moieties consisting of two cycles having two ring atoms in common, i.e., the bridge separating the two rings is either a single bond or a chain of one or two ring atoms, and to spirocyclic moieties, i.e., the two rings are connected via one common ring atom. Some non-limiting examples of heterocyclyl groups include azetidinyl, piperidyl, pyrrolidinyl, oxetanyl, 5-azaspiro[2.5]octan-5-yl, piperidyl, 3,3a,4,5,6,6a-hexahydro-1H-cyclopenta[c]pyrrol-2-yl, 2-azaspiro[3.3]heptan-2-yl, 2,6-diazaspiro[3.3]heptanyl, 2-azaspiro[3.5]nonan-2-yl, 1,2-dihydropyridiynl, piperidyl, pyrrolidinyl, tetrahydrothiophenyl, and thietanyl.

The term “hydroxy” refers to an —OH group.

The term “cyano” refers to a —CN (nitrile) group.

The term “amino” refers to an —NH₂ group.

The term “carboxy” refers to a —COOH group (i.e., a carboxylic acid group).

The term “alkoxycarbonyl” refers to a —C(O)—O—C₁-C₆-alkyl group (i.e., a carboxylic acid ester group).

The term “oxo” refers to a double bonded oxygen (═O).

The term “carbamoyl” refers to a group H₂N—C(O)—.

The term “haloalkyl” refers to an alkyl group, wherein at least one of the hydrogen atoms of the alkyl group has been replaced by a halogen atom, preferably fluoro. Preferably, “haloalkyl” refers to an alkyl group wherein 1, 2 or 3 hydrogen atoms of the alkyl group have been replaced by a halogen atom, most preferably fluoro. Particularly preferred, yet non-limiting examples of haloalkyl are trifluoromethyl, difluoromethyl, 1,1-difluoroethyl, 2,2-difluoroethyl, and 2,2,2-trifluoroethyl.

The term “haloalkoxy” refers to an alkoxy group, wherein at least one of the hydrogen atoms of the alkoxy group has been replaced by a halogen atom, preferably fluoro. Preferably, “haloalkoxy” refers to an alkoxy group wherein 1, 2 or 3 hydrogen atoms of the alkoxy group have been replaced by a halogen atom, most preferably fluoro. Particularly preferred, yet non-limiting examples of haloalkoxy are trifluoromethoxy, difluoromethoxy, 2,2,2-trifluoro-1,1-dimethyl-ethoxy, (1,1,1-trifluoropropan-2-yl)oxy, and 2,2,2-trifluoroethoxy.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like.

The compounds of formula (I) can contain several asymmetric centers and can be present in the form of optically pure enantiomers, mixtures of enantiomers such as, for example, racemates, optically pure diastereioisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The abbreviation “MAGL” refers to the enzyme monoacylglycerol lipase. The terms “MAGL” and “monoacylglycerol lipase” are used herein interchangeably.

The term “treatment” as used herein includes: (1) inhibiting the state, disorder or condition (e.g. arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

The term “prophylaxis” as used herein includes: preventing or delaying the appearance of clinical symptoms of the state, disorder or condition developing in a mammal and especially a human that may be afflicted with or predisposed to the state, disorder or condition but does not yet experience or display clinical or subclinical symptoms of the state, disorder or condition.

The term “neuroinflammation” as used herein relates to acute and chronic inflammation of the nervous tissue, which is the main tissue component of the two parts of the nervous system; the brain and spinal cord of the central nervous system (CNS), and the branching peripheral nerves of the peripheral nervous system (PNS). Chronic neuroinflammation is associated with neurodegenerative diseases such as Alzheimer's disease, Parkinson's disease and multiple sclerosis. Acute neuroinflammation usually follows injury to the central nervous system immediately, e.g., as a result of traumatic brain injury (TBI).

The term “traumatic brain injury” (“TBI”, also known as “intracranial injury”), relates to damage to the brain resulting from external mechanical force, such as rapid acceleration or deceleration, impact, blast waves, or penetration by a projectile.

The term “neurodegenerative diseases” relates to diseases that are related to the progressive loss of structure or function of neurons, including death of neurons. Examples of neurodegenerative diseases include, but are not limited to, multiple sclerosis, Alzheimer's disease, Parkinson's disease and amyotrophic lateral sclerosis.

The term “mental disorders” (also called mental illnesses or psychiatric disorders) relates to behavioral or mental patterns that may cause suffering or a poor ability to function in life. Such features may be persistent, relapsing and remitting, or occur as a single episode. Examples of mental disorders include, but are not limited to, anxiety and depression.

The term “pain” relates to an unpleasant sensory and emotional experience associated with actual or potential tissue damage. Examples of pain include, but are not limited to, nociceptive pain, chronic pain (including idiopathic pain), neuropathic pain including chemotherapy induced neuropathy, phantom pain and phsychogenic pain. A particular example of pain is neuropathic pain, which is caused by damage or disease affecting any part of the nervous system involved in bodily feelings (i.e., the somatosensory system). In one embodiment, “pain” is neuropathic pain resulting from amputation or thoracotomy. In one embodiment, “pain” is chemotherapy induced neuropathy.

The term “neurotoxicity” relates to toxicity in the nervous system. It occurs when exposure to natural or artificial toxic substances (neurotoxins) alter the normal activity of the nervous system in such a way as to cause damage to nervous tissue. Examples of neurotoxicity include, but are not limited to, neurotoxicity resulting from exposure to substances used in chemotherapy, radiation treatment, drug therapies, drug abuse, and organ transplants, as well as exposure to heavy metals, certain foods and food additives, pesticides, industrial and/or cleaning solvents, cosmetics, and some naturally occurring substances.

The term “cancer” refers to a disease characterized by the presence of a neoplasm or tumor resulting from abnormal uncontrolled growth of cells (such cells being “cancer cells”). As used herein, the term cancer explicitly includes, but is not limited to, hepatocellular carcinoma, colon carcinogenesis and ovarian cancer. The term “mammal” as used herein includes both humans and non-humans and includes but is not limited to humans, non-human primates, canines, felines, murines, bovines, equines, and porcines. In a particularly preferred embodiment, the term “mammal” refers to humans.

### Compounds of the Invention

In a first aspect, the present invention provides a compound of Formula (I)

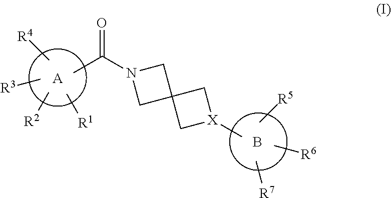

or a pharmaceutically acceptable salt thereof, wherein:
X is CR⁸ or N;
A is selected from C₆-C₁₄-aryl, C₃-C₁₀-cycloalkyl, 5- to 14-membered heteroaryl, and 3- to 14-membered heterocyclyl;
B is a heteroaryl selected from B-1 to B-10:

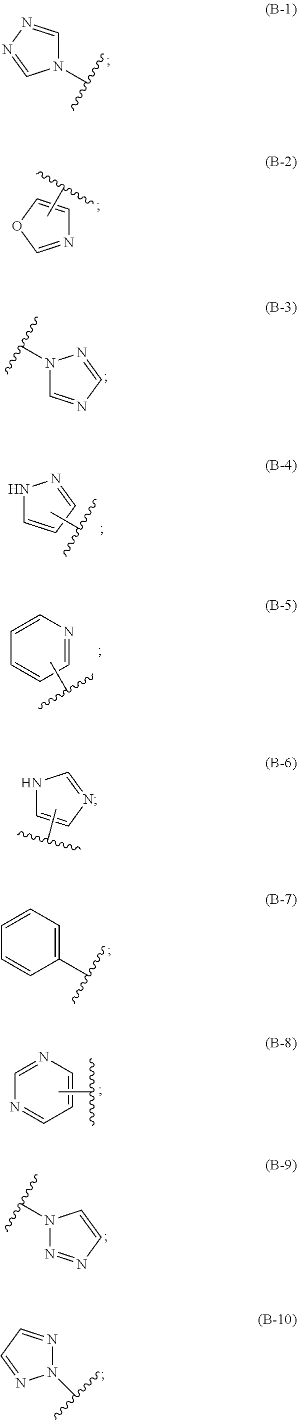

wherein the wavy line indicates the point of attachment to the remainder of formula (I);
C is selected from C₆-C₁₄-aryl, C₃-C₁₀-cycloalkyl, 5- to 14-membered heteroaryl, and 3- to 14-membered heterocyclyl;
D is selected from C₃-C₁₀-cycloalkyl, 3- to 14-membered heterocyclyl, C₆-C₁₄-aryl and 5- to 14-membered heteroaryl;
E is selected from C₃-C₁₀-cycloalkyl and 3- to 14-membered heterocyclyl;
L¹ is selected from a covalent bond, —CR¹²R¹³—, —CH₂O—, —OCH₂—, —CH₂NH—, —NHCH₂—, —CH₂OCH₂—, —O—, —NH—, —CH₂CH₂—, —CH═CH—, [symbol] , —SO₂NH—, —NHSO₂—, —SO₂NHCH₂—, —CH₂NHSO₂—, —SO₂—, —CH₂SO₂—, —SO₂CH₂—, —(CH₂)₂SO₂—, —SO₂(CH₂)₂—, carbonyl, —NHC(O)— and —C(O)NH—;
L² is selected from a covalent bond, —CH₂—, —CH₂NH—, —NHCH₂—, —NH—, —N(C₁-C₆-alkyl)- and —SO₂—;
L³ is selected from a covalent bond and —CH₂—;

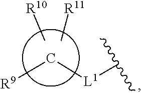

R¹ is selected from hydrogen, halogen, a group, C₁-C₆-alkyl, halo-C₁-C₆-alkyl, C₁-C₆-alkoxy, halo-C₁-C₆-alkoxy, C₁-C₆-alkyl-SO₂NH—, C₃-C₁₀-cycloalkyl-C₁-C₆-alkyl-S(O)₂—, C₁-C₆-alkyl-SO₂—, halo-C₁-C₆-alkyl-S(O)₂—, (C₁-C₆-alkyl)₂N—SO₂—, and halo-C₁-C₆-alkyl-C(O)—;
R², R³, and R⁴ are each independently selected from hydrogen, halogen, C₁-C₆-alkyl, halo-C₁-C₆-alkyl, and 3- to 14-membered heterocyclyl;
R⁵ and R⁶ are each independently selected from hydrogen, halogen, cyano, hydroxy, amino, C₁-C₆-alkyl, C₁-C₆-alkoxy, halo-C₁-C₆-alkyl, halo-C₁-C₆-alkoxy, C₃-C₁₀-cycloalkyl, and 3- to 14-membered heterocyclyl; wherein said C₃-C₁₀-cycloalkyl and 3- to 14-membered heterocyclyl are optionally substituted with 1, 2 or 3 substituents selected from C₁-C₆-alkyl, halo-C₁-C₆-alkyl, C₁-C₆-alkoxy, halo-C₁-C₆-alkoxy, halogen, cyano, amino, and hydroxy;
R⁷ is absent or is selected from hydrogen, halogen, cyano, hydroxy, amino, C₁-C₆-alkyl, C₁-C₆-alkoxy, halo-C₁-C₆-alkyl, and halo-C₁-C₆-alkoxy;
R⁸ is selected from hydrogen, halogen, cyano, C₁-C₆-alkyl, C₁-C₆-alkoxy, and hydroxy;
R⁹ is selected from hydrogen, C₁-C₆-alkyl, C₁-C₆-alkoxy, halo-C₁-C₆-alkyl, halo-C₁-C₆-alkoxy, halogen, cyano, SF₅, C₁-C₆-alkyl-SO₂—, halo-C₁-C₆-alkyl-SO₂—, (C₁-C₆-alkyl)₂-PO—, amino, carboxy, carboxy-C₁-C₆-alkyl, C₁-C₆-alkoxycarbonyl, C₁-C₆-alkoxycarbonyl-C₁-C₆-alkyl-, NH₂SO₂—, carbamoyl, C₁-C₆-alkyl-C(O)NH—, halo-C₁-C₆-alkyl-NHC(O)—, oxo, a group

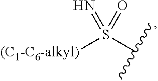

a group

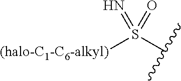

and a group

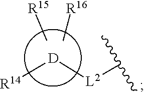

R¹⁰ and R¹¹ are each independently selected from hydrogen, halogen, cyano, C₁-C₆-alkyl, C₁-C₆-alkoxy, halo-C₁-C₆-alkyl, and oxo;
R¹² is selected from hydrogen, carbamoyl, C₁-C₆-alkyl-NHC(O)—, and halo-C₆-C₁₄-aryl and R¹³ is hydrogen; or
R¹² and R¹³, taken together with the carbon atom to which they are attached, form a C₃-C₁₀-cycloalkyl;
R¹⁴ is selected from hydrogen, C₁-C₆-alkyl, halo-C₁-C₆-alkyl, C₁-C₆-alkoxy, halo-C₁-C₆-alkoxy, halogen, cyano, amino, carbamoyl, hydroxy, oxo, C₁-C₆-alkyl-SO₂—, a group

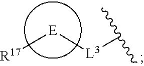

R¹⁵ is selected from hydrogen, halogen, hydroxy, oxo, C₁-C₆-alkyl;
R¹⁶ is selected from hydrogen and halogen; and R¹⁷ is selected from hydrogen, C₁-C₆-alkyl, and halo-C₁-C₆-alkyl.

In one embodiment, the present invention provides a compound of formula (I) as described herein, or a pharmaceutically acceptable salt thereof, wherein:
X is CR⁸;
A is selected from C₆-C₁₄-aryl, C₃-C₁₀-cycloalkyl, 5- to 14-membered heteroaryl, and 3- to 14-membered heterocyclyl;
B is a heteroaryl selected from B-1 to B-6:

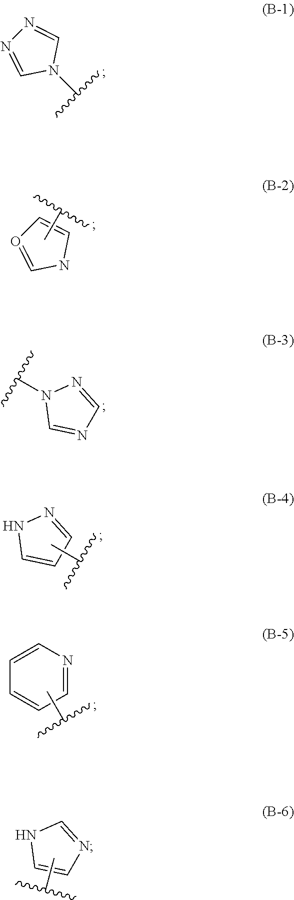

wherein the wavy line indicates the point of attachment to the remainder of formula (I);
C is selected from C₆-C₁₄-aryl, C₃-C₁₀-cycloalkyl, 5- to 14-membered heteroaryl, and 3- to 14-membered heterocyclyl;
L¹ is selected from a covalent bond, —CR¹²R¹³—, —CH₂O—, —OCH₂—, —CH₂NH—, —NHCH₂—, —CH₂OCH₂—, —O—, —NH—, [symbol] , —SO₂NH—, —NHSO₂—, —SO₂NHCH₂—, —CH₂NHSO₂—, —SO₂—, —CH₂SO₂—, —SO₂CH₂—, —(CH₂)₂SO₂—, —SO₂(CH₂)₂—, carbonyl, —NHC(O)— and —C(O)NH—;
R¹ is selected from hydrogen, halogen, a group

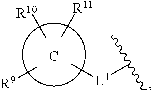

C₁-C₆-alkyl, halo-C₁-C₆-alkyl, C₁-C₆-alkoxy, halo-C₁-C₆-alkoxy, C₁-C₆-alkyl-SO₂NH—, C₃-C₁₀-cycloalkyl-C₁-C₆-alkyl-S(O)₂—, C₁-C₆-alkyl-SO₂—, halo-C₁-C₆-alkyl-S(O)₂—, (C₁-C₆-alkyl)₂N—SO₂—, and halo-C₁-C₆-alkyl-C(O)—;
R², R³, and R⁴ are each independently selected from hydrogen, halogen, C₁-C₆-alkyl, halo-C₁-C₆-alkyl, and 3- to 14-membered heterocyclyl;
R⁵ and R⁶ are each independently selected from hydrogen, halogen, cyano, hydroxy, amino, C₁-C₆-alkyl, C₁-C₆-alkoxy, halo-C₁-C₆-alkyl, halo-C₁-C₆-alkoxy, C₃-C₁₀-cycloalkyl, and 3- to 14-membered heterocyclyl;
   wherein C₃-C₁₀-cycloalkyl and 3- to 14-membered heterocyclyl are optionally substituted with 1, 2 or 3 substituents selected from C₁-C₆-alkyl, halo-C₁-C₆-alkyl, C₁-C₆-alkoxy, halo-C₁-C₆-alkoxy, halogen, cyano, amino, and hydroxy;
R⁷ is absent or is selected from hydrogen, halogen, cyano, hydroxy, amino, C₁-C₆-alkyl, C₁-C₆-alkoxy, halo-C₁-C₆-alkyl, and halo-C₁-C₆-alkoxy;
R⁸ is selected from hydrogen, halogen, cyano, C₁-C₆-alkyl, C₁-C₆-alkoxy, and hydroxy;
R⁹ is selected from hydrogen, C₁-C₆-alkyl, C₁-C₆-alkoxy, halo-C₁-C₆-alkyl, halo-C₁-C₆-alkoxy, halogen, cyano, SF₅, C₃-C₁₀-cycloalkyl, C₃-C₁₀-cycloalkyl-C₁-C₆-alkyl-, 3- to 14-membered heterocyclyl, C₆-C₁₄-aryl, C₁-C₆-alkyl-SO₂—, amino, carboxy, carboxy-C₁-C₆-alkyl, C₁-C₆-alkoxycarbonyl, C₁-C₆-alkoxycarbonyl-C₁-C₆-alkyl-, NH₂SO₂—, carbamoyl, C₁-C₆-alkyl-C(O)NH—, halo-C₁-C₆-alkyl-NHC(O)— and oxo; wherein C₃-C₁₀-cycloalkyl, 3- to 14-membered heterocyclyl, and C₆-C₁₄-aryl are optionally substituted with 1, 2, or 3 substituents selected from C₁-C₆-alkyl, halo-C₁-C₆-alkyl, C₁-C₆-alkoxy, halo-C₁-C₆-alkoxy, 3- to 14-membered heterocyclyl, halogen, cyano, amino, and hydroxy;
R¹⁰ and R¹¹ are each independently selected from hydrogen, halogen, cyano, C₁-C₆-alkyl, C₁-C₆-alkoxy, halo-C₁-C₆-alkyl, and oxo;
R¹² is selected from hydrogen, carbamoyl, C₁-C₆-alkyl-NHC(O)—, and halo-C₆-C₁₄-aryl and R¹³ is hydrogen; or
R¹² and R¹³, taken together with the carbon atom to which they are attached, form a C₃-C₁₀-cycloalkyl.

In one embodiment, the present invention provides a compound of formula (I) as described herein, or a pharmaceutically acceptable salt thereof, wherein:
A is selected from C₆-C₁₄-aryl, C₃-C₁₀-cycloalkyl, 5- to 14-membered heteroaryl, and 3- to 14-membered heterocyclyl;
C is selected from C₆-C₁₄-aryl, C₃-C₁₀-cycloalkyl, 5- to 14-membered heteroaryl, and 3- to 14-membered heterocyclyl;
L¹ is selected from a covalent bond, —CR¹²R¹³—, —CH₂O—, —CH₂NH—, —CH₂OCH₂—, —O—, —NH—, [symbol] , —SO₂NH—, —NHSO₂—, —SO₂NHCH₂—, —CH₂NHSO₂—, —SO₂—, —CH₂SO₂—, —(CH₂)₂SO₂—, carbonyl, and —C(O)NH—;
R¹ is selected from a group

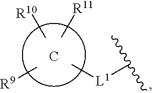

halo-C₁-C₆-alkoxy, C₁-C₆-alkyl-SO₂NH—, C₃-C₁₀-cycloalkyl-C₁-C₆-alkyl-S(O)₂—, C₁-C₆-alkyl-SO₂—, halo-C₁-C₆-alkyl-S(O)₂—, (C₁-C₆-alkyl)₂N—SO₂—, and halo-C₁-C₆-alkyl-C(O)—;
R² is selected from hydrogen, halogen, C₁-C₆-alkyl, halo-C₁-C₆-alkyl, and 3- to 14-membered heterocyclyl;
R³ is selected from hydrogen and halogen;
R⁴ is hydrogen;
R⁹ is selected from hydrogen, C₁-C₆-alkyl, C₁-C₆-alkoxy, halo-C₁-C₆-alkyl, halo-C₁-C₆-alkoxy, halogen, cyano, SF₅, C₃-C₁₀-cycloalkyl, C₃-C₁₀-cycloalkyl-C₁-C₆-alkyl-, 3- to 14-membered heterocyclyl, C₆-C₁₄-aryl, C₁-C₆-alkyl-SO₂—, amino, carboxy, carboxy-C₁-C₆-alkyl, C₁-C₆-alkoxycarbonyl, C₁-C₆-alkoxycarbonyl-C₁-C₆-alkyl-, NH₂SO₂—, carbamoyl, C₁-C₆-alkyl-C(O)NH—, halo-C₁-C₆-alkyl-NHC(O)— and oxo; wherein C₃-C₁₀-cycloalkyl, 3- to 14-membered heterocyclyl, and C₆-C₁₄-aryl are optionally substituted with 1 or 2 substituents selected from halo-C₁-C₆-alkyl, 3- to 14-membered heterocyclyl, halogen, and hydroxy;
R¹⁰ is selected from hydrogen, halogen, cyano, C₁-C₆-alkyl, C₁-C₆-alkoxy, halo-C₁-C₆-alkyl, and oxo;
R¹¹ is selected from hydrogen and halogen; and R¹² is selected from hydrogen, carbamoyl, C₁-C₆-alkyl-NHC(O)—, and halo-C₆-C₁₄-aryl and R¹³ is hydrogen; or
R¹² and R¹³, taken together with the carbon atom to which they are attached, form a C₃-C₁₀-cycloalkyl.

In a preferred embodiment, the present invention provides a compound of formula (I) as described herein, or a pharmaceutically acceptable salt thereof, wherein:
A is selected from C₆-C₁₄-aryl, 5- to 14-membered heteroaryl, and 3- to 14-membered heterocyclyl;
C is selected from C₆-C₁₄-aryl, C₃-C₁₀-cycloalkyl, and 5- to 14-membered heteroaryl;
L¹ is selected from a covalent bond, —CR¹²R¹³—, —CH₂O—, —O—, —SO₂NH—, and —SO₂—;
R¹ is a group

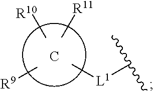

R² is selected from hydrogen and C₁-C₆-alkyl;
R³, R⁴, R¹², and R¹³ are all hydrogen;
R⁹ is selected from C₁-C₆-alkyl, halo-C₁-C₆-alkyl, halo-C₁-C₆-alkoxy, SF₅, C₃-C₁₀-cycloalkyl, 3- to 14-membered heterocyclyl, and C₁-C₆-alkyl-SO₂—; wherein C₃-C₁₀-cycloalkyl and 3- to 14-membered heterocyclyl are optionally substituted with 1 or 2 substituents selected from halo-C₁-C₆-alkyl and hydroxy;
R¹⁰ is selected from hydrogen, halogen and C₁-C₆-alkoxy; and R¹¹ is selected from hydrogen and halogen.

In a particularly preferred embodiment, the present invention provides a compound of formula (I) as described herein, or a pharmaceutically acceptable salt thereof, wherein:
A is selected from phenyl, pyridyl, azetidinyl, 2-azaspiro[3.3]heptan-2-yl, 2,6-diazaspiro[3.3]heptanyl, and 2-azaspiro[3.5]nonan-2-yl;
C is selected from phenyl, cyclopropyl, pyridyl, 1,2,4-oxadiazolyl, pyrazinyl, and pyrimidinyl;
L¹ is selected from a covalent bond, —CR¹²R¹³—, —CH₂O—, —O—, —SO₂NH—, and —SO₂—;
R¹ is a group

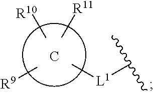

R² is selected from hydrogen and methyl;
R³, R⁴, R¹², and R¹³ are all hydrogen;
R⁹ is selected from tert-butyl, CF₃, CF₃O, SF₅, cyclopropyl, azetidinyl, pyrrolidinyl, and methylsulfonyl;
   wherein cyclopropyl, azetidinyl, and pyrrolidinyl are optionally substituted with 1 or 2 substituents selected from CF₃ and hydroxy;
R¹⁰ is selected from hydrogen, fluoro, chloro and methoxy; and
R¹¹ is selected from hydrogen and fluoro.

In one embodiment, the present invention provides a compound of formula (I) as described herein, or a pharmaceutically acceptable salt thereof, wherein:

A is selected from

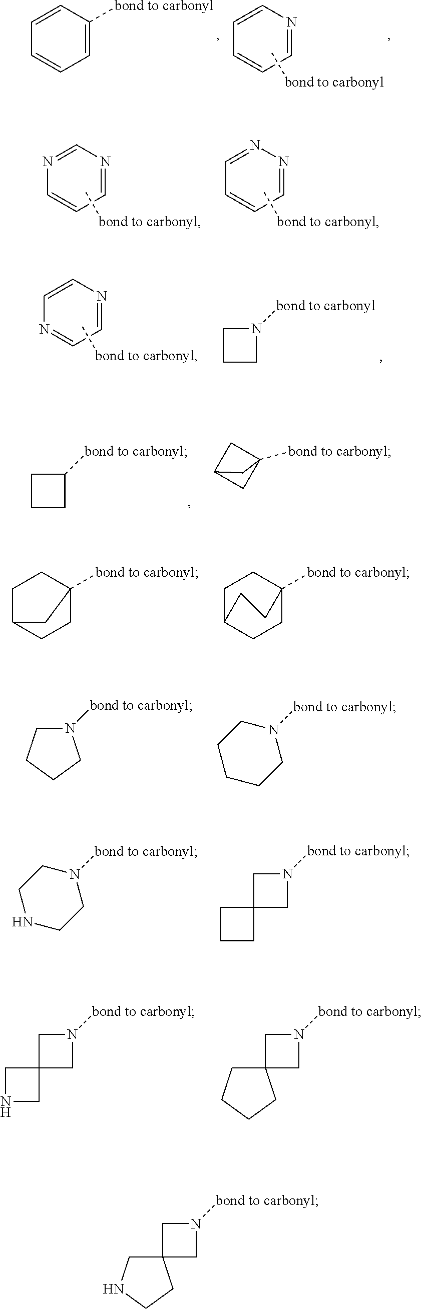

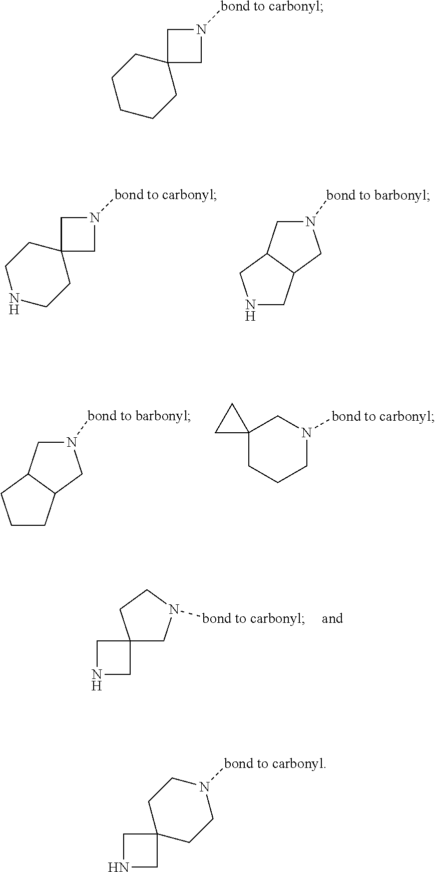

In one embodiment, the present invention provides a compound of formula (I) as described herein, or a pharmaceutically acceptable salt thereof, wherein:

A is selected from

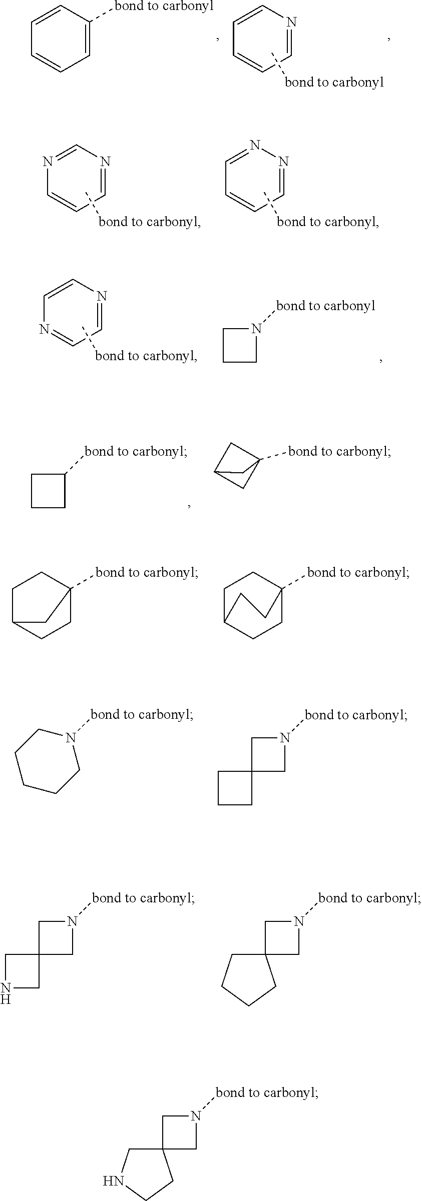

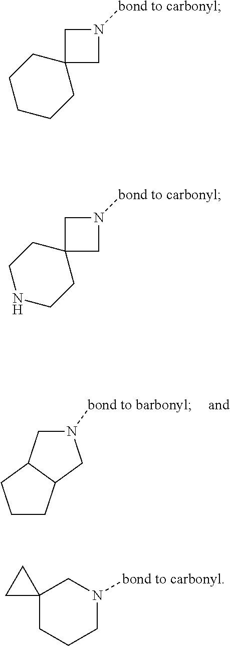

In a preferred embodiment, the present invention provides a compound of formula (I) as described herein, or a pharmaceutically acceptable salt thereof, wherein:

A is selected from

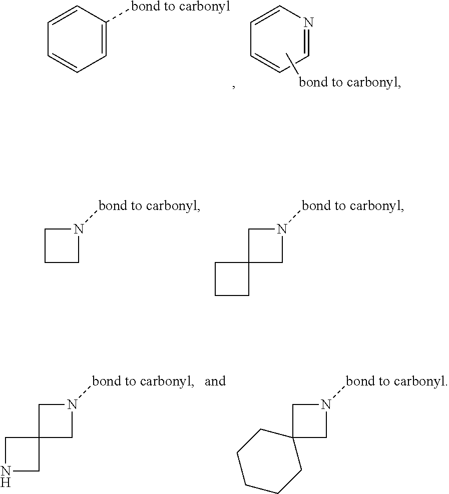

In one embodiment, the present invention provides a compound of formula (I) as described herein, or a pharmaceutically acceptable salt thereof, wherein:

A is selected from

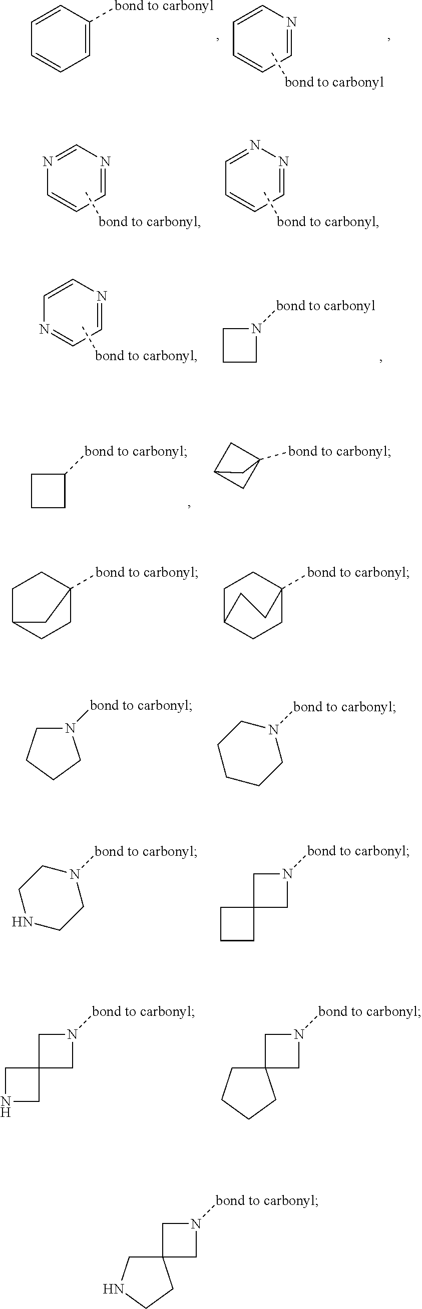

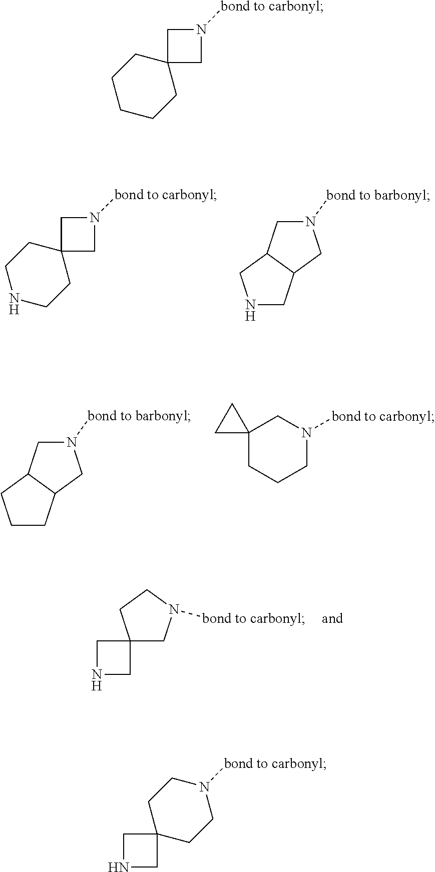

C is selected from C₆-C₁₄-aryl, C₃-C₁₀-cycloalkyl, 5- to 14-membered heteroaryl, and 3- to 14-membered heterocyclyl;
D is selected from C₃-C₁₀-cycloalkyl, 3- to 14-membered heterocyclyl, C₆-C₁₄-aryl and 5- to 14-membered heteroaryl;
E is selected from C₃-C₁₀-cycloalkyl, and 3- to 14-membered heterocyclyl;
L¹ is selected from a covalent bond, —CR¹²R¹³—, —CH₂O—, —CH₂NH—, —CH₂OCH₂—, —O—, —NH—, [symbol] , —SO₂NH—, —NHSO₂—, —SO₂NHCH₂—, —CH₂NHSO₂—, —SO₂—, —CH₂SO₂—, —(CH₂)₂SO₂—, carbonyl, and —C(O)NH—;
L² is selected from a covalent bond, —CH₂—, —CH₂NH—, —NHCH₂—, —NH—, —N(C₁-C₆-alkyl)- and —SO₂—;
L³ is selected from a covalent bond and —CH₂—;
R¹ is selected from a group

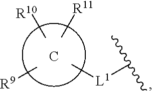

halo-C₁-C₆-alkoxy, C₁-C₆-alkyl-SO₂NH—, C₃-C₁₀-cycloalkyl-C₁-C₆-alkyl-S(O)₂—, C₁-C₆-alkyl-SO₂—, halo-C₁-C₆-alkyl-S(O)₂—, (C₁-C₆-alkyl)₂N—SO₂—, and halo-C₁-C₆-alkyl-C(O)—;
R² is selected from hydrogen, halogen, C₁-C₆-alkyl, halo-C₁-C₆-alkyl, and 3- to 14-membered heterocyclyl;
R³ is selected from hydrogen and halogen;
R⁴ is hydrogen;
R⁹ is selected from hydrogen, C₁-C₆-alkyl, C₁-C₆-alkoxy, halo-C₁-C₆-alkyl, halo-C₁-C₆-alkoxy, halogen, cyano, SF₅, C₁-C₆-alkyl-SO₂—, halo-C₁-C₆-alkyl-SO₂—, (C₁-C₆-alkyl)₂-PO—, amino, carboxy, carboxy-C₁-C₆-alkyl, C₁-C₆-alkoxycarbonyl, C₁-C₆-alkoxycarbonyl-C₁-C₆-alkyl-, NH₂SO₂—, carbamoyl, C₁-C₆-alkyl-C(O)NH—, halo-C₁-C₆-alkyl-NHC(O)—, oxo, a group

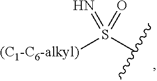

a group

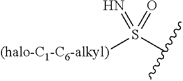

and a group

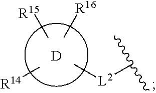

wherein C₃-C₁₀-cycloalkyl, 3- to 14-membered heterocyclyl, and C₆-C₁₄-aryl are optionally substituted with 1 or 2 substituents selected from halo-C₁-C₆-alkyl, 3- to 14-membered heterocyclyl, halogen, and hydroxy;
R¹⁰ is selected from hydrogen, halogen, cyano, C₁-C₆-alkyl, C₁-C₆-alkoxy, halo-C₁-C₆-alkyl, and oxo;
R¹¹ is selected from hydrogen and halogen;
R¹² is selected from hydrogen, carbamoyl, C₁-C₆-alkyl-NHC(O)—, and halo-C₆-C₁₄-aryl and R¹³ is hydrogen; or
R¹² and R¹³, taken together with the carbon atom to which they are attached, form a C₃-C₁₀-cycloalkyl;
R¹⁴ is selected from hydrogen, C₁-C₆-alkyl, halo-C₁-C₆-alkyl, C₁-C₆-alkoxy, halo-C₁-C₆-alkoxy, halogen, cyano, amino, carbamoyl, hydroxy, oxo, C₁-C₆-alkyl-SO₂—, a group

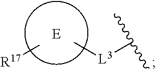

R¹⁵ is selected from hydrogen, halogen, hydroxy, oxo, and C₁-C₆-alkyl;
R¹⁶ is selected from hydrogen and halogen; and R¹⁷ is selected from hydrogen, C₁-C₆-alkyl, and halo-C₁-C₆-alkyl.

In one embodiment, the present invention provides a compound of formula (I) as described herein, or a pharmaceutically acceptable salt thereof, wherein:
A is selected from,

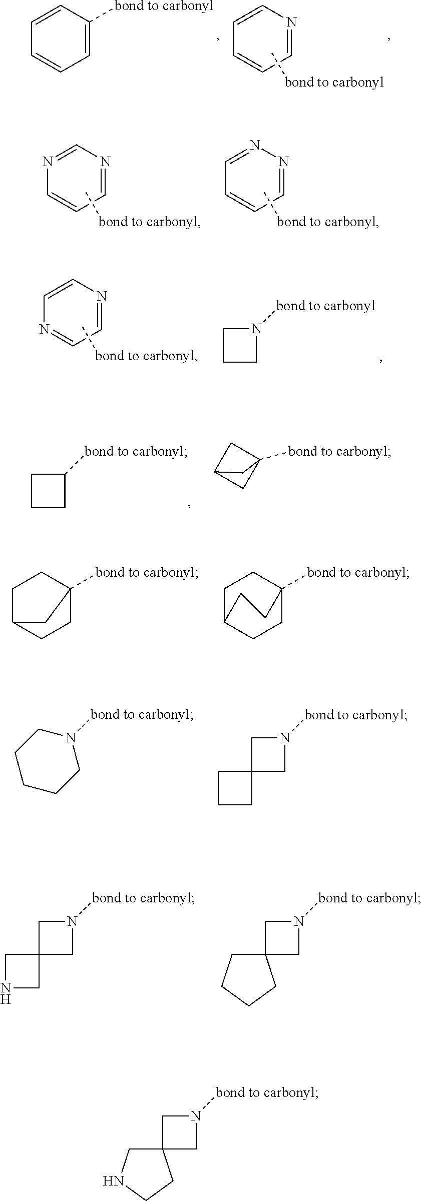

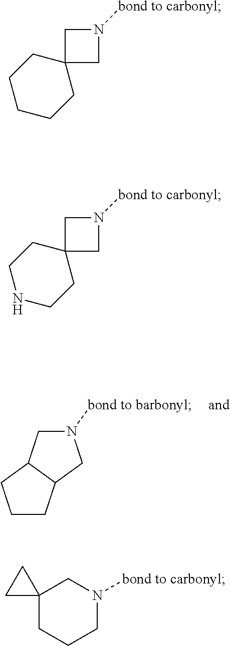

C is selected from C₆-C₁₄-aryl, C₃-C₁₀-cycloalkyl, 5- to 14-membered heteroaryl, and 3- to 14-membered heterocyclyl;
D is selected from C₃-C₁₀-cycloalkyl, 3- to 14-membered heterocyclyl, C₆-C₁₄-aryl and 5- to 14-membered heteroaryl;
E is selected from C₃-C₁₀-cycloalkyl, and 3- to 14-membered heterocyclyl;
L¹ is selected from a covalent bond, —CR¹²R¹³—, —CH₂O—, —CH₂NH—, —CH₂OCH₂—, —O—, —NH—, [symbol] , —SO₂NH—, —NHSO₂—, —SO₂NHCH₂—, —CH₂NHSO₂—, —SO₂—, —CH₂SO₂—, —(CH₂)₂SO₂—, carbonyl, and —C(O)NH—;
L² is selected from a covalent bond, —CH₂—, —CH₂NH—, —NHCH₂—, —NH—, —N(C₁-C₆-alkyl)- and —SO₂—;
L³ is selected from a covalent bond and —CH₂—;
R¹ is selected from a group

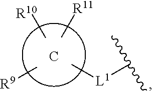

halo-C₁-C₆-alkoxy, C₁-C₆-alkyl-SO₂NH—, C₃-C₁₀-cycloalkyl-C₁-C₆-alkyl-S(O)₂—, C₁-C₆-alkyl-SO₂—, halo-C₁-C₆-alkyl-S(O)₂—, (C₁-C₆-alkyl)₂N—SO₂—, and halo-C₁-C₆-alkyl-C(O)—;
R² is selected from hydrogen, halogen, C₁-C₆-alkyl, halo-C₁-C₆-alkyl, and 3- to 14-membered heterocyclyl;
R³ is selected from hydrogen and halogen;
R⁴ is hydrogen;
R⁹ is selected from hydrogen, C₁-C₆-alkyl, C₁-C₆-alkoxy, halo-C₁-C₆-alkyl, halo-C₁-C₆-alkoxy, halogen, cyano, SF₅, C₁-C₆-alkyl-SO₂—, halo-C₁-C₆-alkyl-SO₂—, (C₁-C₆-alkyl)₂-PO—, amino, carboxy, carboxy-C₁-C₆-alkyl, C₁-C₆-alkoxycarbonyl, C₁-C₆-alkoxycarbonyl-C₁-C₆-alkyl-, NH₂SO₂—, carbamoyl, C₁-C₆-alkyl-C(O)NH—, halo-C₁-C₆-alkyl-NHC(O)—, oxo, a group

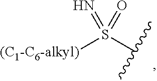

a group

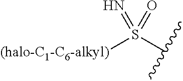

and a group

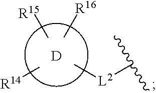

wherein C₃-C₁₀-cycloalkyl, 3- to 14-membered heterocyclyl, and C₆-C₁₄-aryl are optionally substituted with 1 or 2 substituents selected from halo-C₁-C₆-alkyl, 3- to 14-membered heterocyclyl, halogen, and hydroxy;
R¹⁰ is selected from hydrogen, halogen, cyano, C₁-C₆-alkyl, C₁-C₆-alkoxy, halo-C₁-C₆-alkyl, and oxo;
R¹¹ is selected from hydrogen and halogen;
R¹² is selected from hydrogen, carbamoyl, C₁-C₆-alkyl-NHC(O)—, and halo-C₆-C₁₄-aryl and R¹³ is hydrogen; or
R¹² and R¹³, taken together with the carbon atom to which they are attached, form a C₃-C₁₀-cycloalkyl;
R¹⁴ is selected from hydrogen, C₁-C₆-alkyl, halo-C₁-C₆-alkyl, C₁-C₆-alkoxy, halo-C₁-C₆-alkoxy, halogen, cyano, amino, carbamoyl, hydroxy, oxo, C₁-C₆-alkyl-SO₂—, a group

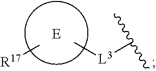

R¹⁵ is selected from hydrogen, halogen, hydroxy, oxo, and C₁-C₆-alkyl;
R¹⁶ is selected from hydrogen and halogen; and
R¹⁷ is selected from hydrogen, C₁-C₆-alkyl, and halo-C₁-C₆-alkyl.

In a preferred embodiment, the present invention provides a compound of formula (I) as described herein, or a pharmaceutically acceptable salt thereof, wherein:

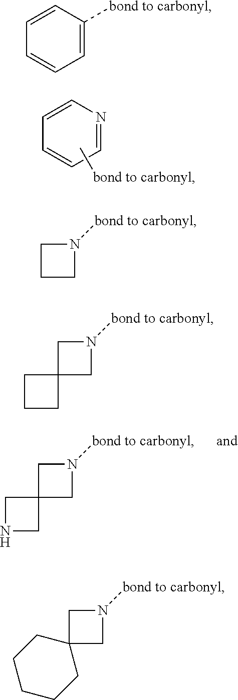

C is selected from C₆-C₁₄-aryl, C₃-C₁₀-cycloalkyl, and 5- to 14-membered heteroaryl;
D is selected from C₃-C₁₀-cycloalkyl and 3- to 14-membered heterocyclyl;
L¹ is selected from a covalent bond, —CR¹²R¹³—, —CH₂O—, —O—, —SO₂NH—, and —SO₂—;
L² is selected from a covalent bond and —CH₂—;
R¹ is a group

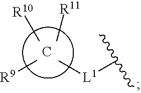

R² is selected from hydrogen and C₁-C₆-alkyl;
R³, R⁴, R¹², and R¹³ are all hydrogen;
R⁹ is selected from halogen, C₁-C₆-alkyl, halo-C₁-C₆-alkyl, halo-C₁-C₆-alkoxy, SF₅, C₁-C₆-alkyl-SO₂—, a group

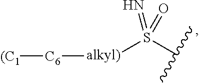

a group

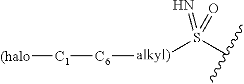

and a group

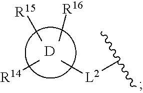

R¹⁰ is selected from hydrogen, halogen, halo-C₁-C₆-alkyl, and C₁-C₆-alkoxy;
R¹¹ is selected from hydrogen and halogen;
R¹⁴ is selected from hydrogen and halo-C₁-C₆-alkyl;
R¹⁵ is selected from hydrogen and hydroxy; and
R¹⁶ is hydrogen.

In one embodiment, the present invention provides a compound of formula (I) as described herein, or a pharmaceutically acceptable salt thereof, wherein:
A is selected from C₆-C₁₄-aryl, C₃-C₁₀-cycloalkyl, 5- to 14-membered heteroaryl, and 3- to 14-membered heterocyclyl;
C is selected from C₆-C₁₄-aryl, C₃-C₁₀-cycloalkyl, 5- to 14-membered heteroaryl, and 3- to 14-membered heterocyclyl;
D is selected from C₃-C₁₀-cycloalkyl, 3- to 14-membered heterocyclyl, C₆-C₁₄-aryl and 5- to 14-membered heteroaryl;
E is selected from C₃-C₁₀-cycloalkyl, and 3- to 14-membered heterocyclyl;
L¹ is selected from a covalent bond, —CR¹²R¹³—, —CH₂O—, —CH₂NH—, —CH₂OCH₂—, —O—, —NH—, [symbol] , —SO₂NH—, —NHSO₂—, —SO₂NHCH₂—, —CH₂NHSO₂—, —SO₂—, —CH₂SO₂—, —(CH₂)₂SO₂—, carbonyl, and —C(O)NH—;
L² is selected from a covalent bond, —CH₂—, —CH₂NH—, —NHCH₂—, —NH—, —N(C₁-C₆-alkyl)- and —SO₂—;
L³ is selected from a covalent bond and —CH₂—;
R¹ is selected from a group

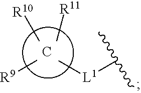

halo-C₁-C₆-alkoxy, C₁-C₆-alkyl-SO₂NH—C₃-C₁₀-cycloalkyl-C₁-C₆-alkyl-S(O)₂—, C₁-C₆-alkyl-SO₂—, halo-C₁-C₆-alkyl-S(O)₂—, (C₁-C₆-alkyl)₂N—SO₂—, and halo-C₁-C₆-alkyl-C(O)—;
R² is selected from hydrogen, halogen, C₁-C₆-alkyl, halo-C₁-C₆-alkyl, and 3- to 14-membered heterocyclyl;
R³ is selected from hydrogen and halogen;
R⁴ is hydrogen;
R⁹ is selected from hydrogen, C₁-C₆-alkyl, C₁-C₆-alkoxy, halo-C₁-C₆-alkyl, halo-C₁-C₆-alkoxy, halogen, cyano, SF₅, C₁-C₆-alkyl-SO₂—, halo-C₁-C₆-alkyl-SO₂—, (C₁-C₆-alkyl)₂-PO—, amino, carboxy, carboxy-C₁-C₆-alkyl, C₁-C₆-alkoxycarbonyl, C₁-C₆-alkoxycarbonyl-C₁-C₆-alkyl-, NH₂SO₂—, carbamoyl, C₁-C₆-alkyl-C(O)NH—, halo-C₁-C₆-alkyl-NHC(O)—, oxo, a group

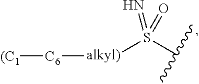

a group

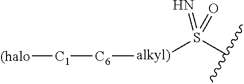

and a group

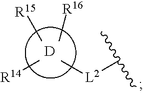

wherein C₃-C₁₀-cycloalkyl, 3- to 14-membered heterocyclyl, and C₆-C₁₄-aryl are optionally substituted with 1 or 2 substituents selected from halo-C₁-C₆-alkyl, 3- to 14-membered heterocyclyl, halogen, and hydroxy;
R¹⁰ is selected from hydrogen, halogen, cyano, C₁-C₆-alkyl, C₁-C₆-alkoxy, halo-C₁-C₆-alkyl, and oxo;
R¹¹ is selected from hydrogen and halogen;
R¹² is selected from hydrogen, carbamoyl, C₁-C₆-alkyl-NHC(O)—, and halo-C₆-C₁₄-aryl and R¹³ is hydrogen; or
R¹² and R¹³, taken together with the carbon atom to which they are attached, form a C₃-C₁₀-cycloalkyl;
R¹⁴ is selected from hydrogen, C₁-C₆-alkyl, halo-C₁-C₆-alkyl, C₁-C₆-alkoxy, halo-C₁-C₆-alkoxy, halogen, cyano, amino, carbamoyl, hydroxy, oxo, C₁-C₆-alkyl-SO₂—, a group

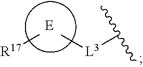

R¹⁵ is selected from hydrogen, halogen, hydroxy, oxo, and C₁-C₆-alkyl;
R¹⁶ is selected from hydrogen and halogen; and
R¹⁷ is selected from hydrogen, C₁-C₆-alkyl, and halo-C₁-C₆-alkyl.

In a preferred embodiment, the present invention provides a compound of formula (I) as described herein, or a pharmaceutically acceptable salt thereof, wherein:
A is selected from C₆-C₁₄-aryl, 5- to 14-membered heteroaryl, and 3- to 14-membered heterocyclyl;
C is selected from C₆-C₁₄-aryl, C₃-C₁₀-cycloalkyl, and 5- to 14-membered heteroaryl;
D is selected from C₃-C₁₀-cycloalkyl and 3- to 14-membered heterocyclyl;
L¹ is selected from a covalent bond, —CR¹²R¹³—, —CH₂O—, —O—, —SO₂NH—, and —SO₂—;
L² is selected from a covalent bond and —CH₂—;

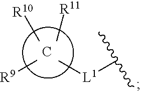

R¹ is a group;
R² is selected from hydrogen and C₁-C₆-alkyl;
R³, R⁴, R¹², and R¹³ are all hydrogen;
R⁹ is selected from halogen, C₁-C₆-alkyl, halo-C₁-C₆-alkyl, halo-C₁-C₆-alkoxy, SF₅, C₁-C₆-alkyl-SO₂—, a group

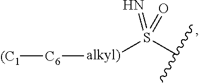

a group

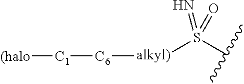

and a group

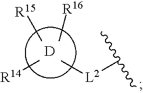

R¹⁰ is selected from hydrogen, halogen, halo-C₁-C₆-alkyl, and C₁-C₆-alkoxy;
R¹¹ is selected from hydrogen and halogen;
R¹⁴ is selected from hydrogen and halo-C₁-C₆-alkyl;
R¹⁵ is selected from hydrogen and hydroxy; and
R¹⁶ is hydrogen.

In a particularly preferred embodiment, the present invention provides a compound of formula (I) as described herein, or a pharmaceutically acceptable salt thereof, wherein:
A is selected from phenyl, pyridyl, azetidinyl, 2-azaspiro[3.3]heptan-2-yl, 2,6-diazaspiro[3.3]heptanyl, and 2-azaspiro[3.5]nonan-2-yl;
C is selected from phenyl, cyclopropyl, pyridyl, 1,2,4-oxadiazolyl, pyrazinyl, and pyrimidinyl;
D is selected from cyclopropyl, azetidinyl, and pyrrolidinyl;
L¹ is selected from a covalent bond, —CR¹²R¹³—, —CH₂O—, —O—, —SO₂NH—, and —SO₂—;
L² is selected from a covalent bond and —CH₂—;
R¹ is a group

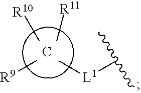

R² is selected from hydrogen and methyl;
R³, R⁴, R¹², and R¹³ are all hydrogen;
R⁹ is selected from fluoro, chloro, tert-butyl, CF₃, CF₃O, SF₅, methylsulfonyl, a group

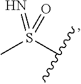 [SMILES: *S(C)(=N)=O]

a group

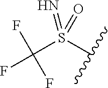 [SMILES: *S(=N)(=O)C(F)(F)F]

and a group

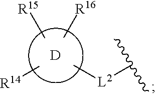

R¹⁰ is selected from hydrogen, fluoro, chloro, CF₃, and methoxy;
R¹¹ is selected from hydrogen and fluoro;
R¹⁴ is selected from hydrogen and CF₃;
R¹⁵ is selected from hydrogen and hydroxy; and
R¹⁶ is hydrogen.

In one embodiment, the present invention provides a compound of formula (I) as described herein, or a pharmaceutically acceptable salt thereof, wherein: B is a heteroaryl selected from B-1 to B-6:

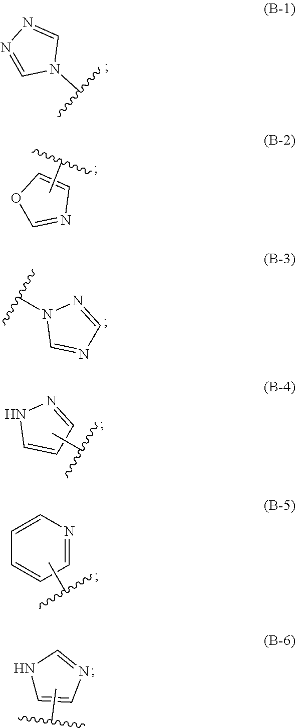

wherein the wavy line indicates the point of attachment to the remainder of formula (I);
R⁵ is selected from hydrogen, halogen, cyano, C₁-C₆-alkyl, C₁-C₆-alkoxy, halo-C₁-C₆-alkyl, C₃-C₁₀-cycloalkyl, and 3- to 14-membered heterocyclyl;
   wherein said C₃-C₁₀-cycloalkyl is optionally substituted with one C₁-C₆-alkyl substituent;
R⁶ is selected from hydrogen and halogen; and
R⁷ is absent or hydrogen.

In a preferred embodiment, the present invention provides a compound of formula (I) as described herein, or a pharmaceutically acceptable salt thereof, wherein:
B is

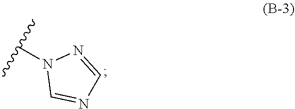 [SMILES: *n1cncn1]

wherein the wavy line indicates the point of attachment to the remainder of formula (I);
R⁵ is selected from C₁-C₆-alkyl and C₃-C₁₀-cycloalkyl;
R⁶ is hydrogen; and
R⁷ is absent.

In a particularly preferred embodiment, the present invention provides a compound of formula (I) as described herein, or a pharmaceutically acceptable salt thereof, wherein:
B is

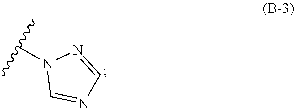 [SMILES: *n1cncn1]

wherein the wavy line indicates the point of attachment to the remainder of formula (I);
R⁵ is selected from ethyl and cyclopropyl;
R⁶ is hydrogen; and
R⁷ is absent.

In one embodiment, the present invention provides a compound of formula (I) as described herein, or a pharmaceutically acceptable salt thereof, wherein:
B is a heteroaryl selected from B-1 to B-10:

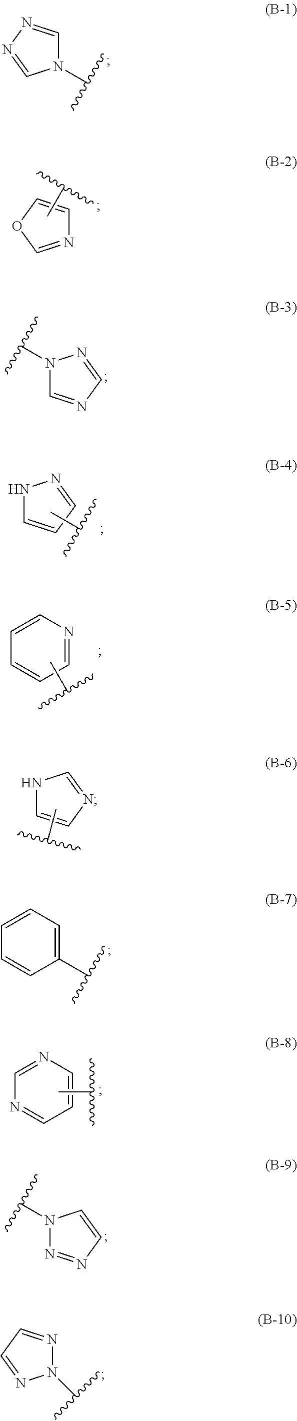

the wavy line indicates the point of attachment to the remainder of formula (I);
R⁵ is selected from hydrogen, halogen, cyano, C₁-C₆-alkyl, C₁-C₆-alkoxy, halo-C₁-C₆-alkyl, C₃-C₁₀-cycloalkyl, and 3- to 14-membered heterocyclyl;
   wherein said C₃-C₁₀-cycloalkyl is optionally substituted with one substituent selected from hydroxy and C₁-C₆-alkyl;
R⁶ is selected from hydrogen and halogen; and
R⁷ is absent or hydrogen.

In a preferred embodiment, the present invention provides a compound of formula (I) as described herein, or a pharmaceutically acceptable salt thereof, wherein:
B is

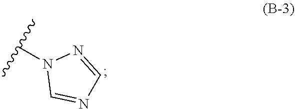 [SMILES: *n1cncn1]

wherein the wavy line indicates the point of attachment to the remainder of formula (I);
R⁵ is selected from C₁-C₆-alkyl, halo-C₁-C₆-alkyl, and C₃-C₁₀-cycloalkyl, wherein said C₃-C₁₀-cycloalkyl is optionally substituted with one hydroxy substituent;
R⁶ is hydrogen; and
R⁷ is absent.

In a particularly preferred embodiment, the present invention provides a compound of formula (I) as described herein, or a pharmaceutically acceptable salt thereof, wherein:
B is

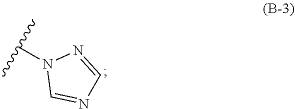 [SMILES: *n1cncn1]

wherein the wavy line indicates the point of attachment to the remainder of formula (I);
R⁵ is selected from ethyl, CF₃, and cyclopropyl, wherein said cyclopropyl is optionally substituted with one hydroxy substituent;
R⁶ is hydrogen; and
R⁷ is absent.

In a preferred embodiment, the present invention provides a compound of formula (I) as described herein, or a pharmaceutically acceptable salt thereof, wherein R⁸ is hydrogen or hydroxy.

In a particularly preferred embodiment, the present invention provides a compound of formula (I) as described herein, or a pharmaceutically acceptable salt thereof, wherein R⁸ is hydrogen.

In one embodiment, the present invention provides a compound of formula (I) as described herein, or a pharmaceutically acceptable salt thereof, wherein:
X is CR⁸ or N; and
R⁸ is hydrogen or hydroxy.

In a preferred embodiment, the present invention provides a compound of formula (I) as described herein, or a pharmaceutically acceptable salt thereof, wherein:
X is CR⁸; and
R⁸ is hydrogen.

In one embodiment, the present invention provides a compound of formula (I) as described herein, or a pharmaceutically acceptable salt thereof, wherein:
A is selected from C₆-C₁₄-aryl, C₃-C₁₀-cycloalkyl, 5- to 14-membered heteroaryl, and 3- to 14-membered heterocyclyl;
B is a heteroaryl selected from B-1 to B-6:

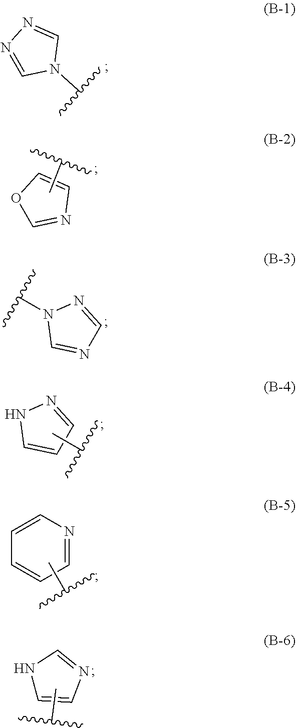

wherein the wavy line indicates the point of attachment to the remainder of formula (I);
C is selected from C₆-C₁₄-aryl, C₃-C₁₀-cycloalkyl, 5- to 14-membered heteroaryl, and 3- to 14-membered heterocyclyl;
L¹ is selected from a covalent bond —CR¹²R¹³—, —CH₂O—, —CH₂NH—, —CH₂OCH₂—, —O—, —NH—, [symbol] , —SO₂NH—, —NHSO₂—, —SO₂NHCH₂—, —CH₂NHSO₂—, —SO₂—, —CH₂SO₂—, —(CH₂)₂SO₂—, carbonyl, and —C(O)NH—;
R¹ is selected from a group

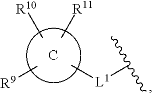

halo-C₁-C₆-alkoxy, C₁-C₆-alkyl-SO₂NH—, C₃-C₁₀-cycloalkyl-C₁-C₆-alkyl-S(O)₂—, C₁-C₆-alkyl-SO₂—, halo-C₁-C₆-alkyl-S(O)₂—, (C₁-C₆-alkyl)₂N—SO₂—, and halo-C₁-C₆-alkyl-C(O)—;
R² is selected from hydrogen, halogen, C₁-C₆-alkyl, halo-C₁-C₆-alkyl, and 3- to 14-membered heterocyclyl;
R³ is selected from hydrogen and halogen;
R⁴ is hydrogen;
R⁵ is selected from hydrogen, halogen, cyano, C₁-C₆-alkyl, C₁-C₆-alkoxy, halo-C₁-C₆-alkyl, C₃-C₁₀-cycloalkyl, and 3- to 14-membered heterocyclyl;
   wherein said C₃-C₁₀-cycloalkyl is optionally substituted with one C₁-C₆-alkyl substituent;
R⁶ is selected from hydrogen and halogen;
R⁷ is absent or hydrogen;
R⁸ is hydrogen or hydroxy;
R⁹ is selected from hydrogen, C₁-C₆-alkyl, C₁-C₆-alkoxy, halo-C₁-C₆-alkyl, halo-C₁-C₆-alkoxy, halogen, cyano, SF₅, C₃-C₁₀-cycloalkyl, C₃-C₁₀-cycloalkyl-C₁-C₆-alkyl-, 3- to 14-membered heterocyclyl, C₆-C₁₄-aryl, C₁-C₆-alkyl-SO₂—, amino, carboxy, carboxy-C₁-C₆-alkyl, C₁-C₆-alkoxycarbonyl, C₁-C₆-alkoxycarbonyl-C₁-C₆-alkyl-, NH₂SO₂—, carbamoyl, C₁-C₆-alkyl-C(O)NH—, halo-C₁-C₆-alkyl-NHC(O)— and oxo;
   wherein C₃-C₁₀-cycloalkyl, 3- to 14-membered heterocyclyl, and C₆-C₁₄-aryl are optionally substituted with 1 or 2 substituents selected from halo-C₁-C₆-alkyl, 3- to 14-membered heterocyclyl, halogen, and hydroxy;
R¹⁰ is selected from hydrogen, halogen, cyano, C₁-C₆-alkyl, C₁-C₆-alkoxy, halo-C₁-C₆-alkyl, and oxo;
R¹¹ is selected from hydrogen and halogen; and
R¹² is selected from hydrogen, carbamoyl, C₁-C₆-alkyl-NHC(O)—, and halo-C₆-C₁₄-aryl and R¹³ is hydrogen; or
R¹² and R¹³, taken together with the carbon atom to which they are attached, form a C₃-C₁₀-cycloalkyl.

In a preferred embodiment, the present invention provides a compound of formula (I) as described herein, or a pharmaceutically acceptable salt thereof, wherein:
A is selected from C₆-C₁₄-aryl, 5- to 14-membered heteroaryl, and 3- to 14-membered heterocyclyl;
B is

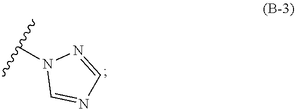 [SMILES: *n1cncn1]

wherein the wavy line indicates the point of attachment to the remainder of formula (I);
C is selected from C₆-C₁₄-aryl, C₃-C₁₀-cycloalkyl, and 5- to 14-membered heteroaryl;
L¹ is selected from a covalent bond, —CR¹²R¹³—, —CH₂O—, —O—, —SO₂NH—, and —SO₂—;
R¹ is a group

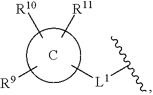

R² is selected from hydrogen and C₁-C₆-alkyl;
R³, R⁴, R⁶, R⁸, R¹², and R¹³ are all hydrogen;
R⁵ is selected from C₁-C₆-alkyl and C₃-C₁₀-cycloalkyl;
R⁷ is absent;
R⁹ is selected from C₁-C₆-alkyl, halo-C₁-C₆-alkyl, halo-C₁-C₆-alkoxy, SF₅, C₃-C₁₀-cycloalkyl, 3- to 14-membered heterocyclyl, and C₁-C₆-alkyl-SO₂—;
   wherein C₃-C₁₀-cycloalkyl and 3- to 14-membered heterocyclyl are optionally substituted with 1 or 2 substituents selected from halo-C₁-C₆-alkyl and hydroxy;
R¹⁰ is selected from hydrogen, halogen and C₁-C₆-alkoxy; and
R¹¹ is selected from hydrogen and halogen.

In a particularly preferred embodiment, the present invention provides a compound of formula (I) as described herein, or a pharmaceutically acceptable salt thereof, wherein:
A is selected from phenyl, pyridyl, azetidinyl, 2-azaspiro[3.3]heptan-2-yl, 2,6-diazaspiro[3.3]heptanyl, and 2-azaspiro[3.5]nonan-2-yl;
B is

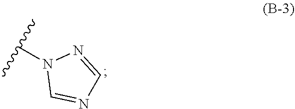 [SMILES: *n1cncn1]

wherein the wavy line indicates the point of attachment to the remainder of formula (I);
C is selected from phenyl, cyclopropyl, pyridyl, 1,2,4-oxadiazolyl, pyrazinyl, and pyrimidinyl;
L¹ is selected from a covalent bond, —CR¹²R¹³—, —CH₂O—, —O—, —SO₂NH—, and —SO₂—;
R¹ is a group

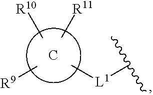

R² is selected from hydrogen and methyl;
R³, R⁴, R⁶, R⁸, R¹², and R¹³ are all hydrogen;
R⁵ is selected from ethyl and cyclpropyl;
R⁷ is absent;
R⁹ is selected from tert-butyl, CF₃, CF₃O, SF₅, cyclopropyl, azetidinyl, pyrrolidinyl, and methylsulfonyl;
   wherein cyclopropyl, azetidinyl, and pyrrolidinyl are optionally substituted with 1 or 2 substituents selected from CF₃ and hydroxy;
R¹⁰ is selected from hydrogen, fluoro, chloro and methoxy; and
R¹¹ is selected from hydrogen and fluoro.

In one embodiment, the present invention provides a compound of formula (I) as described herein, or a pharmaceutically acceptable salt thereof, wherein:
X is CR⁸ or N;
A is selected from C₆-C₁₄-aryl, C₃-C₁₀-cycloalkyl, 5- to 14-membered heteroaryl, and 3- to 14-membered heterocyclyl;
B is a heteroaryl selected from B-1 to B-10:

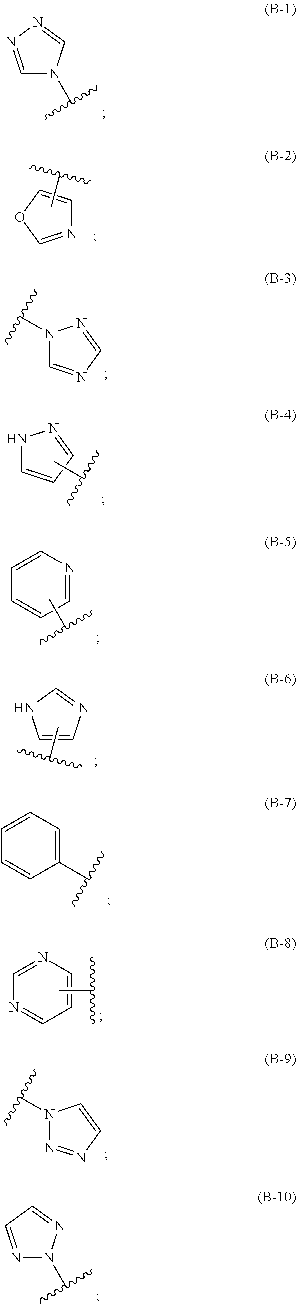

wherein the wavy line indicates the point of attachment to the remainder of formula (I);
C is selected from C₆-C₁₄-aryl, C₃-C₁₀-cycloalkyl, 5- to 14-membered heteroaryl, and 3- to 14-membered heterocyclyl;
D is selected from C₃-C₁₀-cycloalkyl, 3- to 14-membered heterocyclyl, C₆-C₁₄-aryl and 5- to 14-membered heteroaryl;
E is selected from C₃-C₁₀-cycloalkyl, and 3- to 14-membered heterocyclyl;
L¹ is selected from a covalent bond, —CR¹²R¹³—, —CH₂O—, —CH₂NH—, —CH₂OCH₂—, —O—, —NH—, [symbol] , —SO₂NH—, —NHSO₂—, —SO₂NHCH₂—, —CH₂NHSO₂—, —SO₂—, —CH₂SO₂—, —(CH₂)₂SO₂—, carbonyl, and —C(O)NH—;
L² is selected from a covalent bond, —CH₂—, —CH₂NH—, —NHCH₂—, —NH—, —N(C₁-C₆-alkyl)- and —SO₂—;
L³ is selected from a covalent bond and —CH₂—;
R¹ is selected from a group

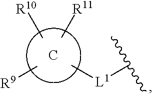

halo-C₁-C₆-alkoxy, C₁-C₆-alkyl-SO₂NH—, C₃-C₁₀-cycloalkyl-C₁-C₆-alkyl-S(O)₂—, C₁-C₆-alkyl-SO₂—, halo-C₁-C₆-alkyl-S(O)₂—, (C₁-C₆-alkyl)₂N—SO₂—, and halo-C₁-C₆-alkyl-C(O)—;
R² is selected from hydrogen, halogen, C₁-C₆-alkyl, halo-C₁-C₆-alkyl, and 3- to 14-membered heterocyclyl;
R³ is selected from hydrogen and halogen;
R⁴ is hydrogen;
R⁵ is selected from hydrogen, halogen, cyano, C₁-C₆-alkyl, C₁-C₆-alkoxy, halo-C₁-C₆-alkyl, C₃-C₁₀-cycloalkyl, and 3- to 14-membered heterocyclyl;
   wherein said C₃-C₁₀-cycloalkyl is optionally substituted with one substituent selected from hydroxy and C₁-C₆-alkyl;
R⁶ is selected from hydrogen and halogen;
R⁷ is absent or hydrogen;
R⁸ is hydrogen or hydroxy;
R⁹ is selected from hydrogen, C₁-C₆-alkyl, C₁-C₆-alkoxy, halo-C₁-C₆-alkyl, halo-C₁-C₆-alkoxy, halogen, cyano, SF₅, C₁-C₆-alkyl-SO₂—, halo-C₁-C₆-alkyl-SO₂—, (C₁-C₆-alkyl)₂-PO—, amino, carboxy, carboxy-C₁-C₆-alkyl, C₁-C₆-alkoxycarbonyl, C₁-C₆-alkoxycarbonyl-C₁-C₆-alkyl-, NH₂SO₂—, carbamoyl, C₁-C₆-alkyl-C(O)NH—, halo-C₁-C₆-alkyl-NHC(O)—, oxo, a group

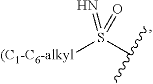

a group

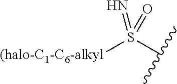

and a group

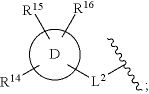

R¹⁰ is selected from hydrogen, halogen, cyano, C₁-C₆-alkyl, C₁-C₆-alkoxy, halo-C₁-C₆-alkyl, and oxo;
R¹¹ is selected from hydrogen and halogen;
R¹² is selected from hydrogen, carbamoyl, C₁-C₆-alkyl-NHC(O)—, and halo-C₆-C₁₄-aryl and R¹³ is hydrogen; or
R¹² and R¹³, taken together with the carbon atom to which they are attached, form a C₃-C₁₀-cycloalkyl;
R¹⁴ is selected from hydrogen, C₁-C₆-alkyl, halo-C₁-C₆-alkyl, C₁-C₆-alkoxy, halo-C₁-C₆-alkoxy, halogen, cyano, amino, carbamoyl, hydroxy, oxo, C₁-C₆-alkyl-SO₂—, a group

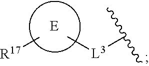

R¹⁵ is selected from hydrogen, halogen, hydroxy, oxo, and C₁-C₆-alkyl;
R¹⁶ is selected from hydrogen and halogen; and
R¹⁷ is selected from hydrogen, C₁-C₆-alkyl, and halo-C₁-C₆-alkyl.

In a preferred embodiment, the present invention provides a compound of formula (I) as described herein, or a pharmaceutically acceptable salt thereof, wherein:
X is CR⁸;
A is selected from C₆-C₁₄-aryl, 5- to 14-membered heteroaryl, and 3- to 14-membered heterocyclyl;
B is

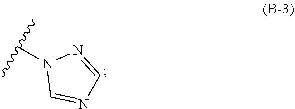 [SMILES: *n1cncn1]

wherein the wavy line indicates the point of attachment to the remainder of formula (I);
C is selected from C₆-C₁₄-aryl, C₃-C₁₀-cycloalkyl, and 5- to 14-membered heteroaryl;
D is selected from C₃-C₁₀-cycloalkyl and 3- to 14-membered heterocyclyl;
L¹ is selected from a covalent bond, —CR¹²R¹³—, —CH₂O—, —O—, —SO₂NH—, and —SO₂—;
L² is selected from a covalent bond and —CH₂—;
R¹ is a group

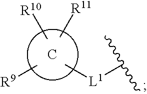

R² is selected from hydrogen and C₁-C₆-alkyl;
R³, R⁴, R⁶, R⁸, R¹², and R¹³ are all hydrogen;
R⁵ is selected from C₁-C₆-alkyl, halo-C₁-C₆-alkyl, and C₃-C₁₀-cycloalkyl, wherein said C₃-C₁₀-cycloalkyl is optionally substituted with one hydroxy substituent;
R⁷ is absent;
R⁹ is selected from halogen, C₁-C₆-alkyl, halo-C₁-C₆-alkyl, halo-C₁-C₆-alkoxy, SF₅, C₁-C₆-alkyl-SO₂—, a group

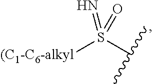

a group

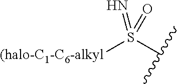

and a group

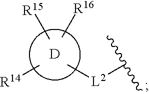

R¹⁰ is selected from hydrogen, halogen, halo-C₁-C₆-alkyl, and C₁-C₆-alkoxy;
R¹¹ is selected from hydrogen and halogen;
R¹⁴ is selected from hydrogen and halo-C₁-C₆-alkyl;
R¹⁵ is selected from hydrogen and hydroxy; and
R¹⁶ is hydrogen.

In a particularly preferred embodiment, the present invention provides a compound of formula (I) as described herein, or a pharmaceutically acceptable salt thereof, wherein:
X is CR⁸;
A is selected from phenyl, pyridyl, azetidinyl, 2-azaspiro[3.3]heptan-2-yl, 2,6-diazaspiro[3.3]heptanyl, and 2-azaspiro[3.5]nonan-2-yl;
B is

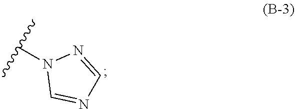 [SMILES: *n1cncn1]

wherein the wavy line indicates the point of attachment to the remainder of formula (I);
C is selected from phenyl, cyclopropyl, pyridyl, 1,2,4-oxadiazolyl, pyrazinyl, and pyrimidinyl;
D is selected from cyclopropyl, azetidinyl, and pyrrolidinyl;
L¹ is selected from a covalent bond, —CR¹²R¹³—, —CH₂O—, —O—, —SO₂NH—, and —SO₂—;
L² is selected from a covalent bond and —CH₂—;
R¹ is a group

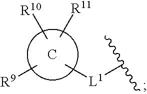

R² is selected from hydrogen and methyl;
R³, R⁴, R⁶, R⁸, R¹², and R¹³ are all hydrogen;
R⁵ is selected from ethyl, CF₃, and cyclpropyl, wherein said cyclopropyl is optionally substituted with one hydroxy substituent;
R⁷ is absent;
R⁹ is selected from fluoro, chloro, tert-butyl, CF₃, CF₃O, SF₅, methylsulfonyl, a group

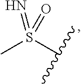 [SMILES: *S(C)(=N)=O]

a group

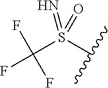 [SMILES: *S(=N)(=O)C(F)(F)F]

and a group

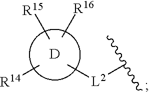

R¹⁰ is selected from hydrogen, fluoro, chloro, CF₃, and methoxy;
R¹¹ is selected from hydrogen and fluoro;
R¹⁴ is selected from hydrogen and CF₃;
R¹⁵ is selected from hydrogen and hydroxy; and
R¹⁶ is hydrogen.

In one embodiment, the present invention provides a compound of formula (I) as described herein, or a pharmaceutically acceptable salt thereof, wherein A is selected from C₆-C₁₄-aryl, C₃-C₁₀-cycloalkyl, 5- to 14-membered heteroaryl, and 3- to 14-membered heterocyclyl.

In one embodiment, the present invention provides a compound of formula (I) as described herein, or a pharmaceutically acceptable salt thereof, wherein B is a heteroaryl selected from B-1 to B-6:

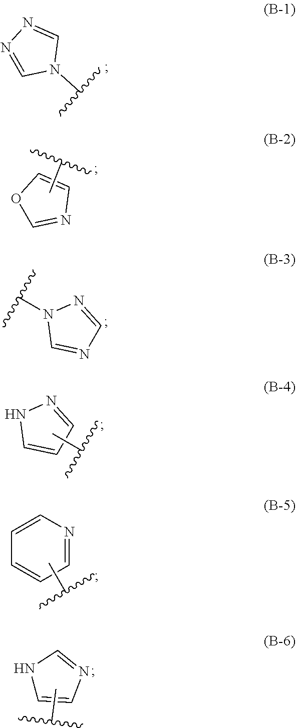

wherein the wavy line indicates the point of attachment to the remainder of formula (I).

In one embodiment, the present invention provides a compound of formula (I) as described herein, or a pharmaceutically acceptable salt thereof, wherein B is a heteroaryl selected from B-1 to B-10:

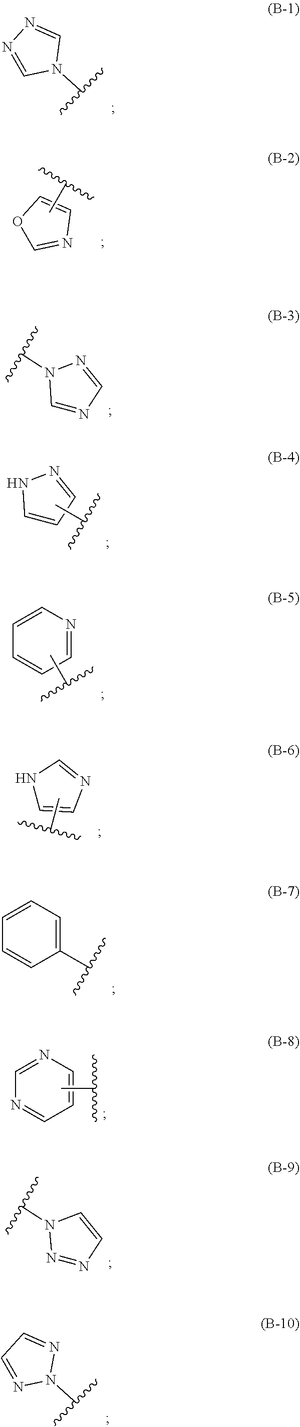

wherein the wavy line indicates the point of attachment to the remainder of formula (I).

In one embodiment, the present invention provides a compound of formula (I) as described herein, or a pharmaceutically acceptable salt thereof, wherein C is selected from C₆-C₁₄-aryl, C₃-C₁₀-cycloalkyl, 5- to 14-membered heteroaryl, and 3- to 14-membered heterocyclyl.

In one embodiment, the present invention provides a compound of formula (I) as described herein, or a pharmaceutically acceptable salt thereof, wherein C is selected from phenyl, bicyclo[1.1.1]pentanyl, pyridyl, pyrimidinyl, pyridazinyl, and pyrazinyl.

In one embodiment, the present invention provides a compound of formula (I) as described herein, or a pharmaceutically acceptable salt thereof, wherein D is selected from cyclopropyl, thietanyl, tetrahydrothiophene, azetidinyl, pyrrolidinyl, piperidyl, oxetanyl, phenyl, 1H-1,2,4-triazolyl, 1H-triazolyl, 4H-1,2,4-triazolyl, and 1,3,4-oxadiazolyl.

In one embodiment, the present invention provides a compound of formula (I) as described herein, or a pharmaceutically acceptable salt thereof, wherein E is selected from C₃-C₁₀-cycloalkyl, and 3- to 14-membered heterocyclyl.

In one embodiment, the present invention provides a compound of formula (I) as described herein, or a pharmaceutically acceptable salt thereof, wherein E is selected from cyclopropyl and cycobutyl.

In one embodiment, the present invention provides a compound of formula (I) as described herein, or a pharmaceutically acceptable salt thereof, wherein L¹ is selected from a covalent [symbol] bond, —CR¹²R¹³—, —CH₂O—, —CH₂NH—, —CH₂OCH₂—, —O—, —NH—, [symbol] , —SO₂NH—, —NHSO₂—, —SO₂NHCH₂—, —CH₂NHSO₂—, —SO₂—, —CH₂SO₂—, —(CH₂)₂SO₂—, carbonyl, and —C(O)NH—, wherein R¹² and R¹³ are as defined herein.

In one embodiment, the present invention provides a compound of formula (I) as described herein, or a pharmaceutically acceptable salt thereof, wherein L² is selected from a covalent bond, —CH₂—, —CH₂NH—, —NHCH₂—, —NH—, —N(C₁-C₆-alkyl)- and —SO₂—.

In one embodiment, the present invention provides a compound of formula (I) as described herein, or a pharmaceutically acceptable salt thereof, wherein L³ is selected from a covalent bond and —CH₂—. In one embodiment, the present invention provides a compound of formula (I) as described herein, or a pharmaceutically acceptable salt thereof, wherein R¹ is selected from a group

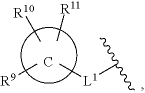

halo-C₁-C₆-alkoxy, C₁-C₆-alkyl-SO₂NH—, C₃-C₁₀-cycloalkyl-C₁-C₆-alkyl-S(O)₂—, C₁-C₆-alkyl-SO₂—, halo-C₁-C₆-alkyl-S(O)₂—, (C₁-C₆-alkyl)₂N—SO₂—, and halo-C₁-C₆-alkyl-C(O)—; wherein R⁹, R¹⁰, R¹, L¹, and C are as defined herein.

In one embodiment, the present invention provides a compound of formula (I) as described herein, or a pharmaceutically acceptable salt thereof, wherein R² is selected from hydrogen, halogen, C₁-C₆-alkyl, halo-C₁-C₆-alkyl, and 3- to 14-membered heterocyclyl.

In one embodiment, the present invention provides a compound of formula (I) as described herein, or a pharmaceutically acceptable salt thereof, wherein R³ is selected from hydrogen and halogen.

In a preferred embodiment, the present invention provides a compound of formula (I) as described herein, or a pharmaceutically acceptable salt thereof, wherein R⁴ is hydrogen.

In one embodiment, the present invention provides a compound of formula (I) as described herein, or a pharmaceutically acceptable salt thereof, wherein R⁵ is selected from hydrogen, halogen, cyano, C₁-C₆-alkyl, C₁-C₆-alkoxy, halo-C₁-C₆-alkyl, C₃-C₁₀-cycloalkyl, and 3- to 14-membered heterocyclyl; wherein said C₃-C₁₀-cycloalkyl is optionally substituted with one C₁-C₆-alkyl substituent.

In one embodiment, the present invention provides a compound of formula (I) as described herein, or a pharmaceutically acceptable salt thereof, wherein R⁵ is selected from hydrogen, halogen, cyano, C₁-C₆-alkyl, C₁-C₆-alkoxy, halo-C₁-C₆-alkyl, C₃-C₁₀-cycloalkyl, and 3- to 14-membered heterocyclyl; wherein said C₃-C₁₀-cycloalkyl is optionally substituted with one substituent selected from hydroxy and C₁-C₆-alkyl.

In one embodiment, the present invention provides a compound of formula (I) as described herein, or a pharmaceutically acceptable salt thereof, wherein R⁶ is selected from hydrogen and halogen.

In one embodiment, the present invention provides a compound of formula (I) as described herein, or a pharmaceutically acceptable salt thereof, wherein R⁷ is absent or hydrogen.

In one embodiment, the present invention provides a compound of formula (I) as described herein, or a pharmaceutically acceptable salt thereof, wherein R⁸ is hydrogen or hydroxy.

In one embodiment, the present invention provides a compound of formula (I) as described herein, or a pharmaceutically acceptable salt thereof, wherein R⁹ is selected from hydrogen, C₁-C₆-alkyl, C₁-C₆-alkoxy, halo-C₁-C₆-alkyl, halo-C₁-C₆-alkoxy, halogen, cyano, SF₅, C₃-C₁₀-cycloalkyl, C₃-C₁₀-cycloalkyl-C₁-C₆-alkyl-, 3- to 14-membered heterocyclyl, C₆-C₁₄-aryl, C₁-C₆-alkyl-SO₂—, amino, carboxy, carboxy-C₁-C₆-alkyl, C₁-C₆-alkoxycarbonyl, C₁-C₆-alkoxycarbonyl-C₁—C₆-alkyl-, NH₂SO₂—, carbamoyl, C₁-C₆-alkyl-C(O)NH—, halo-C₁-C₆-alkyl-NHC(O)— and oxo; wherein C₃-C₁₀-cycloalkyl, 3- to 14-membered heterocyclyl, and C₆-C₁₄-aryl are optionally substituted with 1 or 2 substituents selected from halo-C₁-C₆-alkyl, 3- to 14-membered heterocyclyl, halogen, and hydroxy.

In one embodiment, the present invention provides a compound of formula (I) as described herein, or a pharmaceutically acceptable salt thereof, wherein R⁹ is selected from hydrogen, C₁-C₆-alkyl, C₁-C₆-alkoxy, halo-C₁-C₆-alkyl, halo-C₁-C₆-alkoxy, halogen, cyano, SF₅, C₁-C₆-alkyl-SO₂—, halo-C₁-C₆-alkyl-SO₂—, (C₁-C₆-alkyl)₂-PO—, amino, carboxy, carboxy-C₁-C₆-alkyl, C₁-C₆-alkoxycarbonyl, C₁-C₆-alkoxycarbonyl-C₁-C₆-alkyl-, NH₂SO₂—, carbamoyl, C₁-C₆-alkyl-C(O)NH—, halo-C₁-C₆-alkyl-NHC(O)—, oxo, a group

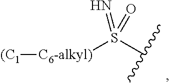

a group

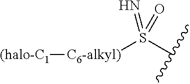

and a group

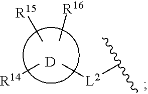

wherein L², D, and R¹⁴ to R¹⁶ are as defined herein.

In one embodiment, the present invention provides a compound of formula (I) as described herein, or a pharmaceutically acceptable salt thereof, wherein R¹⁰ is selected from hydrogen, halogen, cyano, C₁-C₆-alkyl, C₁-C₆-alkoxy, halo-C₁-C₆-alkyl, and oxo.

In one embodiment, the present invention provides a compound of formula (I) as described herein, or a pharmaceutically acceptable salt thereof, wherein R¹¹ is selected from hydrogen and halogen.

In one embodiment, the present invention provides a compound of formula (I) as described herein, or a pharmaceutically acceptable salt thereof, wherein R¹² is selected from hydrogen, carbamoyl, C₁-C₆-alkyl-NHC(O)—, and halo-C₆-C₁₄-aryl.

In a preferred embodiment, the present invention provides a compound of formula (I) as described herein, or a pharmaceutically acceptable salt thereof, wherein R¹³ is hydrogen.

In one embodiment, the present invention provides a compound of formula (I) as described herein, or a pharmaceutically acceptable salt thereof, wherein R¹² and R¹³, taken together with the carbon atom to which they are attached, form a C₃-C₁₀-cycloalkyl.

In a preferred embodiment, the present invention provides a compound of formula (I) as described herein, or a pharmaceutically acceptable salt thereof, wherein R¹⁴ is selected from hydrogen, C₁-C₆-alkyl, halo-C₁-C₆-alkyl, C₁-C₆-alkoxy, halo-C₁-C₆-alkoxy, halogen, cyano, amino, carbamoyl, hydroxy, oxo, C₁-C₆-alkyl-SO₂—, and a group

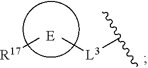

wherein L³, E, and R¹⁷ are as defined herein.

In a preferred embodiment, the present invention provides a compound of formula (I) as described herein, or a pharmaceutically acceptable salt thereof, wherein R¹⁵ is selected from hydrogen, halogen, hydroxy, oxo, and C₁-C₆-alkyl.

In a preferred embodiment, the present invention provides a compound of formula (I) as described herein, or a pharmaceutically acceptable salt thereof, wherein R¹⁶ is selected from hydrogen and halogen.

In a preferred embodiment, the present invention provides a compound of formula (I) as described herein, or a pharmaceutically acceptable salt thereof, wherein R¹⁷ is selected from hydrogen, C₁-C₆-alkyl, and halo-C₁-C₆-alkyl.

In a preferred embodiment, the present invention provides a compound of formula (I) as described herein, or a pharmaceutically acceptable salt thereof, wherein A is selected from C₆-C₁₄-aryl, 5- to 14-membered heteroaryl, and 3- to 14-membered heterocyclyl.

In a preferred embodiment, the present invention provides a compound of formula (I) as described herein, or a pharmaceutically acceptable salt thereof, wherein B is

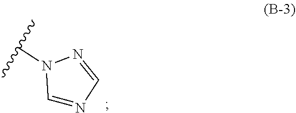 [SMILES: *n1cncn1]

wherein the wavy line indicates the point of attachment to the remainder of formula (I).

In a preferred embodiment, the present invention provides a compound of formula (I) as described herein, or a pharmaceutically acceptable salt thereof, wherein C is selected from C₆-C₁₄-aryl, C₃-C₁₀-cycloalkyl, and 5- to 14-membered heteroaryl.

In a preferred embodiment, the present invention provides a compound of formula (I) as described herein, or a pharmaceutically acceptable salt thereof, wherein D is selected from C₃-C₁₀-cycloalkyl and 3- to 14-membered heterocyclyl.

In a preferred embodiment, the present invention provides a compound of formula (I) as described herein, or a pharmaceutically acceptable salt thereof, wherein L¹ is selected from a covalent bond, —CR¹²R¹³—, —CH₂O—, —O—, —SO₂NH—, and —SO₂—, wherein R¹² and R¹³ are as defined herein.

In a preferred embodiment, the present invention provides a compound of formula (I) as described herein, or a pharmaceutically acceptable salt thereof, wherein L² is selected from a covalent bond and —CH₂—.

In a preferred embodiment, the present invention provides a compound of formula (I) as described herein, or a pharmaceutically acceptable salt thereof, wherein R¹ is a group

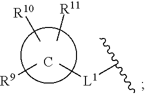

wherein R⁹, R¹⁰, R¹¹, L¹, and C are as defined herein.

In a preferred embodiment, the present invention provides a compound of formula (I) as described herein, or a pharmaceutically acceptable salt thereof, wherein R² is selected from hydrogen and C₁-C₆-alkyl.

In a preferred embodiment, the present invention provides a compound of formula (I) as described herein, or a pharmaceutically acceptable salt thereof, wherein R³ is hydrogen.

In a preferred embodiment, the present invention provides a compound of formula (I) as described herein, or a pharmaceutically acceptable salt thereof, wherein R⁶ is hydrogen.

In a preferred embodiment, the present invention provides a compound of formula (I) as described herein, or a pharmaceutically acceptable salt thereof, wherein R⁸ is hydrogen.

In a preferred embodiment, the present invention provides a compound of formula (I) as described herein, or a pharmaceutically acceptable salt thereof, wherein R¹² is hydrogen.

In a preferred embodiment, the present invention provides a compound of formula (I) as described herein, or a pharmaceutically acceptable salt thereof, wherein R⁵ is selected from C₁-C₆-alkyl and C₃-C₁₀-cycloalkyl.

In a preferred embodiment, the present invention provides a compound of formula (I) as described herein, or a pharmaceutically acceptable salt thereof, wherein R⁵ is selected from C₁-C₆-alkyl, halo-C₁-C₆-alkyl, and C₃-C₁₀-cycloalkyl, wherein said C₃-C₁₀-cycloalkyl is optionally substituted with one hydroxy substituent.

In a preferred embodiment, the present invention provides a compound of formula (I) as described herein, or a pharmaceutically acceptable salt thereof, wherein R⁷ is absent.

In a preferred embodiment, the present invention provides a compound of formula (I) as described herein, or a pharmaceutically acceptable salt thereof, wherein R⁹ is selected from C₁-C₆-alkyl, halo-C₁-C₆-alkyl, halo-C₁-C₆-alkoxy, SF₅, C₃-C₁₀-cycloalkyl, 3- to 14-membered heterocyclyl, and C₁-C₆-alkyl-SO₂—; wherein C₃-C₁₀-cycloalkyl and 3- to 14-membered heterocyclyl are optionally substituted with 1 or 2 substituents selected from halo-C₁-C₆-alkyl and hydroxy.

In a preferred embodiment, the present invention provides a compound of formula (I) as described herein, or a pharmaceutically acceptable salt thereof, wherein R⁹ is selected from halogen, C₁-C₆-alkyl, halo-C₁-C₆-alkyl, halo-C₁-C₆-alkoxy, SF₅, C₁-C₆-alkyl-SO₂—, a group

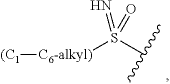

a group

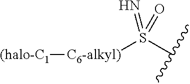

and a group

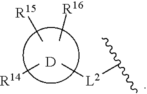

In a preferred embodiment, the present invention provides a compound of formula (I) as described herein, or a pharmaceutically acceptable salt thereof, wherein R¹⁰ is selected from hydrogen, halogen and C₁-C₆-alkoxy.

In a preferred embodiment, the present invention provides a compound of formula (I) as described herein, or a pharmaceutically acceptable salt thereof, wherein R¹⁰ is selected from hydrogen, halogen, halo-C₁-C₆-alkyl, and C₁-C₆-alkoxy.

In a preferred embodiment, the present invention provides a compound of formula (I) as described herein, or a pharmaceutically acceptable salt thereof, wherein R¹¹ is selected from hydrogen and halogen.

In a preferred embodiment, the present invention provides a compound of formula (I) as described herein, or a pharmaceutically acceptable salt thereof, wherein R¹⁴ is selected from hydrogen and halo-C₁-C₆-alkyl.

In a preferred embodiment, the present invention provides a compound of formula (I) as described herein, or a pharmaceutically acceptable salt thereof, wherein R¹⁵ is selected from hydrogen and hydroxy.

In a preferred embodiment, the present invention provides a compound of formula (I) as described herein, or a pharmaceutically acceptable salt thereof, wherein R¹⁶ is hydrogen.

In a particularly preferred embodiment, the present invention provides a compound of formula (I) as described herein, or a pharmaceutically acceptable salt thereof, wherein A is selected from phenyl, pyridyl, azetidinyl, 2-azaspiro[3.3]heptan-2-yl, 2,6-diazaspiro[3.3]heptanyl, and 2-azaspiro[3.5]nonan-2-yl.

In a particularly preferred embodiment, the present invention provides a compound of formula (I) as described herein, or a pharmaceutically acceptable salt thereof, wherein C is selected from phenyl, cyclopropyl, pyridyl, 1,2,4-oxadiazolyl, pyrazinyl, and pyrimidinyl.

In a particularly preferred embodiment, the present invention provides a compound of formula (I) as described herein, or a pharmaceutically acceptable salt thereof, wherein D is selected from phenyl, cyclopropyl, pyridyl, 1,2,4-oxadiazolyl, pyrazinyl, and pyrimidinyl.

In a particularly preferred embodiment, the present invention provides a compound of formula (I) as described herein, or a pharmaceutically acceptable salt thereof, wherein R² is selected from hydrogen and methyl.

In a particularly preferred embodiment, the present invention provides a compound of formula (I) as described herein, or a pharmaceutically acceptable salt thereof, wherein R⁵ is selected from ethyl and cyclpropyl.

In a particularly preferred embodiment, the present invention provides a compound of formula (I) as described herein, or a pharmaceutically acceptable salt thereof, wherein R⁵ is selected from ethyl, CF₃, and cyclpropyl, wherein said cyclopropyl is optionally substituted with one hydroxy substituent.

In a particularly preferred embodiment, the present invention provides a compound of formula (I) as described herein, or a pharmaceutically acceptable salt thereof, wherein R⁷ is absent.

In a particularly preferred embodiment, the present invention provides a compound of formula (I) as described herein, or a pharmaceutically acceptable salt thereof, wherein R⁹ is selected from tert-butyl, CF₃, CF₃O, SF₅, cyclopropyl, azetidinyl, pyrrolidinyl, and methylsulfonyl; wherein cyclopropyl, azetidinyl, and pyrrolidinyl are optionally substituted with 1 or 2 substituents selected from CF₃ and hydroxy.

In a particularly preferred embodiment, the present invention provides a compound of formula (I) as described herein, or a pharmaceutically acceptable salt thereof, wherein R⁹ is selected from fluoro, chloro, tert-butyl, CF₃, CF₃O, SF₅, methylsulfonyl, a group

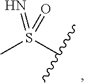 [SMILES: *S(C)(=N)=O]

a group

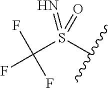 [SMILES: *S(=N)(=O)C(F)(F)F]

and a group

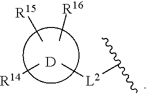

In a particularly preferred embodiment, the present invention provides a compound of formula (I) as described herein, or a pharmaceutically acceptable salt thereof, wherein R¹⁰ is selected from hydrogen, fluoro, chloro and methoxy.

In a particularly preferred embodiment, the present invention provides a compound of formula (I) as described herein, or a pharmaceutically acceptable salt thereof, wherein R¹⁰ is selected from hydrogen, fluoro, chloro, CF₃, and methoxy.

In a particularly preferred embodiment, the present invention provides a compound of formula (I) as described herein, or a pharmaceutically acceptable salt thereof, wherein R¹¹ is selected from hydrogen and fluoro.

In a particularly preferred embodiment, the present invention provides a compound of formula (I) as described herein, or a pharmaceutically acceptable salt thereof, wherein R¹⁴ is selected from hydrogen and CF₃.

In a preferred embodiment, the present invention provides a compound of formula (I) as described herein, or a pharmaceutically acceptable salt thereof, wherein
A is selected from C₆-C₁₄-aryl and 3- to 14-membered heterocyclyl;
B is

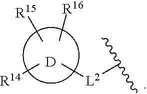

wherein the wavy line indicates the point of attachment to the remainder of formula (I);
C is selected from C₆-C₁₄-aryl and 5- to 14-membered heteroaryl;
L¹ is selected from a covalent bond and —CR¹²R¹³—;
R¹ is a group

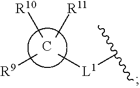

R², R³, R⁴, R⁶, R⁸, R¹¹, R¹², and R¹³ are each hydrogen;
R⁵ is C₃-C₁₀-cycloalkyl;
R⁷ is absent;
R⁹ is selected from C₁-C₆-alkyl, halogen and C₃-C₁₀-cycloalkyl; wherein C₃-C₁₀-cycloalkyl is substituted with a halo-C₁-C₆-alkyl; and
R¹⁰ is selected from hydrogen and halogen.

In a particularly preferred embodiment, the present invention provides a compound of formula (I) as described herein, or a pharmaceutically acceptable salt thereof, wherein
A is selected from phenyl, azetidinyl, and 2-azaspiro[3.3]heptan-2-y;
B is

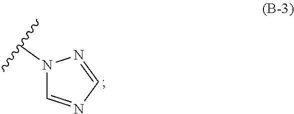 [SMILES: *n1cncn1]

wherein the wavy line indicates the point of attachment to the remainder of formula (I);
C is selected from phenyl and 1,2,4-oxadiazolyl;
L¹ is selected from a covalent bond and —CR¹²R¹³—;
R¹ is a group

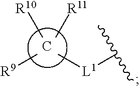

R², R³, R⁴, R⁶, R⁸, R¹¹, R¹², and R¹³ are each hydrogen;
R⁵ is cyclopropyl;
R⁷ is absent;
R⁹ is selected from tert-butyl, fluoro and cyclopropyl; wherein cyclopropyl is substituted with a CF₃; and
R¹⁰ is selected from hydrogen and fluoro.

In a preferred embodiment, the present invention provides a compound of formula (I) as described herein, or a pharmaceutically acceptable salt thereof, wherein A is selected from C₆-C₁₄-aryl and 3- to 14-membered heterocyclyl.

In a preferred embodiment, the present invention provides a compound of formula (I) as described herein, or a pharmaceutically acceptable salt thereof, wherein C is selected from C₆-C₁₄-aryl and 5- to 14-membered heteroaryl.

In a preferred embodiment, the present invention provides a compound of formula (I) as described herein, or a pharmaceutically acceptable salt thereof, wherein L¹ is selected from a covalent bond and —CR¹²R¹³—.

In a preferred embodiment, the present invention provides a compound of formula (I) as described herein, or a pharmaceutically acceptable salt thereof, wherein R² is hydrogen.

In a preferred embodiment, the present invention provides a compound of formula (I) as described herein, or a pharmaceutically acceptable salt thereof, wherein R¹¹ is hydrogen.

In a preferred embodiment, the present invention provides a compound of formula (I) as described herein, or a pharmaceutically acceptable salt thereof, wherein R⁵ is C₃-C₁₀-cycloalkyl.

In a preferred embodiment, the present invention provides a compound of formula (I) as described herein, or a pharmaceutically acceptable salt thereof, wherein R⁹ is selected from C₁-C₆-alkyl, halogen and C₃-C₁₀-cycloalkyl; wherein C₃-C₁₀-cycloalkyl is substituted with a halo-C₁-C₆-alkyl.

In a preferred embodiment, the present invention provides a compound of formula (I) as described herein, or a pharmaceutically acceptable salt thereof, wherein R¹⁰ is selected from hydrogen and halogen.

In a particularly preferred embodiment, the present invention provides a compound of formula (I) as described herein, or a pharmaceutically acceptable salt thereof, wherein A is selected from phenyl, azetidinyl, and 2-azaspiro[3.3]heptan-2-y.

In a particularly preferred embodiment, the present invention provides a compound of formula (I) as described herein, or a pharmaceutically acceptable salt thereof, wherein C is selected from phenyl and 1,2,4-oxadiazolyl.

In a particularly preferred embodiment, the present invention provides a compound of formula (I) as described herein, or a pharmaceutically acceptable salt thereof, wherein R⁵ is cyclopropyl.

In a particularly preferred embodiment, the present invention provides a compound of formula (I) as described herein, or a pharmaceutically acceptable salt thereof, wherein R⁹ is selected from tert-butyl, fluoro and cyclopropyl; wherein cyclopropyl is substituted with a CF₃.

In a particularly preferred embodiment, the present invention provides a compound of formula (I) as described herein, or a pharmaceutically acceptable salt thereof, wherein R¹⁰ is selected from hydrogen and fluoro.

In a preferred embodiment, the present invention provides a compound of formula (I) as described herein, or a pharmaceutically acceptable salt thereof, wherein A is 3- to 14-membered heterocyclyl.

In a particularly preferred embodiment, the present invention provides a compound of formula (I) as described herein, or a pharmaceutically acceptable salt thereof, wherein A is azetidinyl.

In a preferred embodiment, the present invention provides a compound of formula (I) as described herein, or a pharmaceutically acceptable salt thereof, wherein C is C₆-C₁₄-aryl.

In a particularly preferred embodiment, the present invention provides a compound of formula (I) as described herein, or a pharmaceutically acceptable salt thereof, wherein C is phenyl.

In a particularly preferred embodiment, the present invention provides a compound of formula (I) as described herein, or a pharmaceutically acceptable salt thereof, wherein L¹ is a covalent bond.

In a preferred embodiment, the present invention provides a compound of formula (I) as described herein, or a pharmaceutically acceptable salt thereof, wherein R⁹ is C₃-C₁₀-cycloalkyl substituted with a halo-C₁-C₆-alkyl.

In a particularly preferred embodiment, the present invention provides a compound of formula (I) as described herein, or a pharmaceutically acceptable salt thereof, wherein R⁹ is cyclopropyl substituted with a CF₃.

In a particularly preferred embodiment, the present invention provides a compound of formula (I) as described herein, or a pharmaceutically acceptable salt thereof, wherein R¹⁰ is hydrogen.

In a particularly preferred embodiment, the present invention provides a compound of formula (I) as described herein, or a pharmaceutically acceptable salt thereof, wherein said compound of formula (I) is a compound of formula (II):

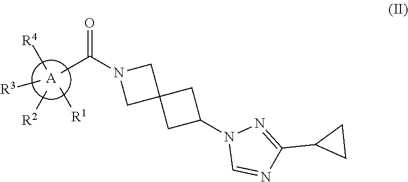

wherein A, R¹, R², R³, and R⁴ are as defined herein.

In one embodiment, the present invention provides a compound of formula (I) as described herein, or a pharmaceutically acceptable salt thereof, wherein A is selected from phenyl, cyclobutyl, 1-bicyclo[1.1.1]pentanyl, norbornanyl, 1-bicyclo[2.2.2]octanyl, pyridyl, pyrimidinyl, pyridazinyl, pyrazinyl, azetidinyl, pyrrolidinyl, 5-azaspiro[2.5]octan-5-yl, piperidyl, 3,3a,4,5,6,6a-hexahydro-1H-cyclopenta[c]pyrrol-2-yl, 2-azaspiro[3.3]heptan-2-yl, 2,6-diazaspiro[3.3]heptanyl, and 2-azaspiro[3.5]nonan-2-yl.

In one embodiment, the present invention provides a compound of formula (I) as described herein, or a pharmaceutically acceptable salt thereof, wherein B is selected from pyrazolyl, imidazolyl, triazolyl, pyridyl, oxazolyl, 4,5,6,7-tetrahydroindazol-2-yl, and 6,7-dihydro-4H-pyrano[4,3-c]pyrazol-2-yl.

In one embodiment, the present invention provides a compound of formula (I) as described herein, or a pharmaceutically acceptable salt thereof, wherein C is selected from phenyl, cyclopropyl, cyclohexyl, 1,2,4-triazolyl, thiazolyl, pyridyl, 1,2,4-oxadiazolyl; 1,3,4-oxadiazolyl, pyrazolyl, pyrazinyl, pyridazinyl, benzofurazan-4-yl, tetrazolyl, isoxazolyl, pyrimidinyl, morpholinyl, 1,2-dihydropyridiynl, piperidyl, pyrrolidinyl, and thietanyl.

In one embodiment, the present invention provides a compound of formula (I) as described herein, or a pharmaceutically acceptable salt thereof, wherein R¹ is selected from a group

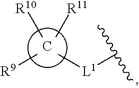

2,2,2-trifluoro-1,1-dimethyl-ethoxy, 2,2,2-trifluoroethoxy, C₁-C₆-alkyl-SO₂NH—, C₃-C₁₀-cycloalkyl-C₁-C₆-alkyl-S(O)₂—, C₁-C₆-alkyl-SO₂—, halo-C₁-C₆-alkyl-S(O)₂—, (C₁-C₆-alkyl)₂N—SO₂—, halo-C₁-C₆-alkyl-C(O)—, wherein R⁹, R¹⁰, R^{D}, L¹, and C are as defined herein.

In one embodiment, the present invention provides a compound of formula (I) as described herein, or a pharmaceutically acceptable salt thereof, wherein R² is selected from hydrogen, fluoro, methyl, CF₃, and oxetanyl.

In one embodiment, the present invention provides a compound of formula (I) as described herein, or a pharmaceutically acceptable salt thereof, wherein R³ is selected from hydrogen and fluoro.

In one embodiment, the present invention provides a compound of formula (I) as described herein, or a pharmaceutically acceptable salt thereof, wherein R⁵ is selected from hydrogen, fluoro, chloro, cyano, methyl, ethyl, methoxy, CF₃, cyclopropyl, cyclobutyl, and azetidinyl; wherein said cyclopropyl and cyclobutyl is optionally substituted with one or more methyl substituents.

In one embodiment, the present invention provides a compound of formula (I) as described herein, or a pharmaceutically acceptable salt thereof, wherein R⁹ is selected from hydrogen, methyl, tert-butyl, 2,2-dimethylpropyl, methoxy, CF₃, difluoroethyl, 1,1-difluoroethyl, 2,2,2-trifluoroethyl, 2,2,2-trifluoroethoxy, difluoromethoxy, CF₃O, (1,1,1-trifluoropropan-2-yl)oxy), fluoro, cyano, SF₅, cyclopropyl, cyclopropyl-CH₂—, oxetanyl, azetidinyl, pyrrolidinyl, phenyl, methylsulfonyl, 2-neopentylsulfonyl, amino, carboxy, 2-methylpropanoic acid, 2,2-dimethylpropanoic acid, methoxycarbonyl, methyl-2,2-dimethylpropanoate, methyl-2-methylpropanoate, NH₂SO₂—, carbamoyl, C₁-C₆-alkyl-C(O)NH—, halo-C₁-C₆-alkyl-NHC(O)— and oxo; wherein cyclopropyl, phenyl, oxetanyl, azetidinyl, and pyrrolidinyl are optionally substituted with 1 to 2 substituents selected from CF₃, morpholinyl, halogen, and hydroxy.

In one embodiment, the present invention provides a compound of formula (I) as described herein, or a pharmaceutically acceptable salt thereof, wherein R¹⁰ is selected from hydrogen, fluoro, chloro, cyano, methyl, methoxy, CF₃, 2,2,2-trifluoroethyl, and oxo.

In a preferred embodiment, the present invention provides a compound of formula (I) as described herein, or a pharmaceutically acceptable salt thereof, wherein:
X is CR⁸;
A is 3- to 14-membered heterocyclyl;
B is

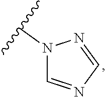 [SMILES: *n1cncn1]

wherein the wavy line indicates the point of attachment to the remainder of formula (I);
C is C₆-C₁₄-aryl;
D is C₃-C₁₀-cycloalkyl;
L¹ is a covalent bond;
L² is a covalent bond;
R¹ is a group

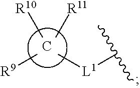

R², R³, R⁴, R⁶, R⁸, R¹⁰, R¹, R¹⁵, R¹⁶, are all hydrogen;
R⁵ is C₃-C₁₀-cycloalkyl;
R⁷ is absent;
R⁹ is a group

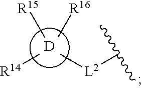

and
R¹⁴ is halo-C₁-C₆-alkyl.

In a particularly preferred embodiment, the present invention provides a compound of formula (I) as described herein, or a pharmaceutically acceptable salt thereof, wherein:
X is CR⁸;
A is azetidinyl;
B is

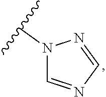 [SMILES: *n1cncn1]

wherein the wavy line indicates the point of attachment to the remainder of formula (I);
C is phenyl;
D is cyclopropyl;
L¹ is a covalent bond;
L² is a covalent bond;
R¹ is a group

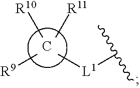

R², R³, R⁴, R⁶, R^{B}, R¹⁰, R¹, R¹⁵, R¹⁶, are all hydrogen;
R⁵ is cyclopropyl;
R⁷ is absent;
R⁹ is a group

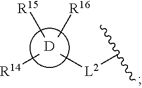

and
R¹⁴ is CF₃.

In a preferred embodiment, the present invention provides a compound of formula (I) as described herein, or a pharmaceutically acceptable salt thereof, wherein:
X is CR⁸;
A is a 3- to 14-membered heterocyclyl;
B is

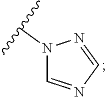 [SMILES: *n1cncn1]

C is a 5- to 14-membered heteroaryl;
L¹ is —CH₂—; and
R¹ to R⁸ are as defined herein.

In a preferred embodiment, the present invention provides a compound of formula (I) as described herein, or a pharmaceutically acceptable salt thereof, wherein:
X is CR⁸;
A is a 3- to 14-membered heterocyclyl;
B is

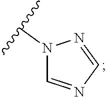 [SMILES: *n1cncn1]

C is a 5- to 14-membered heteroaryl;
L¹ is —CH₂—;
R⁵ is C₃-C₁₀-cycloalkyl;
R⁶ is hydrogen;
R⁷ is absent; and
R¹ to R⁴ and R⁸ are as defined herein.

In a particularly preferred embodiment, the present invention provides a compound of formula (I) as described herein, or a pharmaceutically acceptable salt thereof, wherein:
X is CR⁸;
A is 2-azaspiro[3.3]heptane;
B is

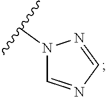 [SMILES: *n1cncn1]

C is a 6-membered heteroaryl;
L¹ is —CH₂—; and
R¹ to R⁸ are as defined herein.

In a particularly preferred embodiment, the present invention provides a compound of formula (I) as described herein, or a pharmaceutically acceptable salt thereof, wherein:
X is CR⁸;
A is a 2-azaspiro[3.3]heptane;
B is

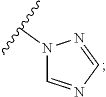 [SMILES: *n1cncn1]

C is a 6-membered heteroaryl;
L¹ is —CH₂—;
R⁵ is cyclopropyl;
R⁶ is hydrogen;
R⁷ is absent; and
R¹ to R⁴ and R⁸ are as defined herein.

In one embodiment, the present invention provides a compound of formula (I) as described herein, or a pharmaceutically acceptable salt thereof, wherein said compound of formula (I) is selected from:
(6-(3-cyclopropyl-1H-1,2,4-triazol-1-yl)-2-azaspiro[3.3]heptan-2-yl)(3-(4-(1-(trifluoromethyl)cyclopropyl)phenyl)azetidin-1-yl)methanone;
(6-(3-cyclopropyl-1H-1,2,4-triazol-1-yl)-2-azaspiro[3.3]heptan-2-yl)(3-(3-((1-(trifluoromethyl)cyclopropyl)methyl)-1,2,4-oxadiazol-5-yl)azetidin-1-yl)methanone;
(4-(5-(tert-butyl)-1,2,4-oxadiazol-3-yl)phenyl)(6-(3-cyclopropyl-1H-1,2,4-triazol-1-yl)-2-azaspiro[3.3]heptan-2-yl)methanone;
(4-(5-(tert-butyl)-1,2,4-oxadiazol-3-yl)phenyl)(6-(3-(trifluoromethyl)-1H-1,2,4-triazol-1-yl)-2-azaspiro[3.3]heptan-2-yl)methanone;
[4-(5-tert-butyl-1,2,4-oxadiazol-3-yl)phenyl]−[6-[3-(trifluoromethyl)pyrazol-1-yl]-2-azaspiro[3.3]heptan-2-yl]methanone;
[4-(5-tert-butyl-1,2,4-oxadiazol-3-yl)phenyl]−[6-(4-chloropyrazol-1-yl)-2-azaspiro[3.3]heptan-2-yl]methanone;
[4-(5-tert-butyl-1,2,4-oxadiazol-3-yl)phenyl]−[6-(4-cyclopropylpyrazol-1-yl)-2-azaspiro[3.3]heptan-2-yl]methanone;
1-[2-[4-(5-tert-butyl-1,2,4-oxadiazol-3-yl)benzoyl]-2-azaspiro[3.3]heptan-6-yl]pyrazole-3-carbonitrile;
[4-(5-tert-butyl-1,2,4-oxadiazol-3-yl)phenyl]−[6-[3-(1-methylcyclopropyl)pyrazol-1-yl]-2-azaspiro[3.3]heptan-2-yl]methanone;
[4-(5-tert-butyl-1,2,4-oxadiazol-3-yl)phenyl]−[6-[5-(1-methylcyclopropyl)pyrazol-1-yl]-2-azaspiro[3.3]heptan-2-yl]methanone;
[4-(5-tert-butyl-1,2,4-oxadiazol-3-yl)phenyl]−[6-(4-methoxypyrazol-1-yl)-2-azaspiro[3.3]heptan-2-yl]methanone;
[4-(5-tert-butyl-1,2,4-oxadiazol-3-yl)phenyl]−[6-(4,5,6,7-tetrahydroindazol-2-yl)-2-azaspiro[3.3]heptan-2-yl]methanone;
[4-(5-tert-butyl-1,2,4-oxadiazol-3-yl)phenyl]−[6-(3-methoxypyrazol-1-yl)-2-azaspiro[3.3]heptan-2-yl]methanone;
[4-(5-tert-butyl-1,2,4-oxadiazol-3-yl)phenyl]−[6-(6,7-dihydro-4H-pyrano[4,3-c]pyrazol-2-yl)-2-azaspiro[3.3]heptan-2-yl]methanone;
[4-(5-tert-butyl-1,2,4-oxadiazol-3-yl)phenyl]−[6-(4-fluoropyrazol-1-yl)-2-azaspiro[3.3]heptan-2-yl]methanone;
[4-(5-tert-butyl-1,2,4-oxadiazol-3-yl)phenyl]−[6-[5-(trifluoromethyl)pyrazol-1-yl]-2-azaspiro[3.3]heptan-2-yl]methanone;
[4-(5-tert-butyl-1,2,4-oxadiazol-3-yl)phenyl]−[6-(5-cyclopropyl-1,2,4-triazol-1-yl)-2-azaspiro[3.3]heptan-2-yl]methanone;
[4-(5-tert-butyl-1,2,4-oxadiazol-3-yl)phenyl]−[6-(6,7-dihydro-4H-pyrano[4,3-c]pyrazol-1-yl)-2-azaspiro[3.3]heptan-2-yl]methanone;
[4-(5-tert-butyl-1,2,4-oxadiazol-3-yl)phenyl]−[6-(5-methoxypyrazol-1-yl)-2-azaspiro[3.3]heptan-2-yl]methanone;
1-[2-[4-(5-tert-butyl-1,2,4-oxadiazol-3-yl)benzoyl]-2-azaspiro[3.3]heptan-6-yl]-1,2,4-triazole-3-carbonitrile;
1-[2-[4-(5-tert-butyl-1,2,4-oxadiazol-3-yl)benzoyl]-2-azaspiro[3.3]heptan-6-yl]pyrazole-4-carbonitrile;
[4-(5-tert-butyl-1,2,4-oxadiazol-3-yl)phenyl]−[6-(4,5,6,7-tetrahydroindazol-1-yl)-2-azaspiro[3.3]heptan-2-yl]methanone;
[4-(5-tert-butyl-1,2,4-oxadiazol-3-yl)phenyl]−[6-(6-methyl-3-pyridyl)-2-azaspiro[3.3]heptan-2-yl]methanone;
[6-(3-cyclopropyl-1,2,4-triazol-1-yl)-2-azaspiro[3.3]heptan-2-yl]−[5-methyl-6-[[1-(trifluoromethyl)cyclopropyl]methoxy]-3-pyridyl]methanone;
[6-(4-cyclopropylimidazol-1-yl)-2-azaspiro[3.3]heptan-2-yl]−[5-methyl-6-[[1-(trifluoromethyl)cyclopropyl]methoxy]-3-pyridyl]methanone;
[6-(3-cyclopropyl-1,2,4-triazol-1-yl)-2-azaspiro[3.3]heptan-2-yl]−[5-fluoro-6-[[1-(trifluoromethyl)cyclopropyl]methoxy]-3-pyridyl]methanone;
[6-(3-cyclopropyl-1,2,4-triazol-1-yl)-2-azaspiro[3.3]heptan-2-yl]−[6-methyl-5-[[1-(trifluoromethyl)cyclopropyl]methoxy]pyrazin-2-yl]methanone;
[6-(4-cyclopropylimidazol-1-yl)-2-azaspiro[3.3]heptan-2-yl]−[5-fluoro-6-[[1-(trifluoromethyl)cyclopropyl]methoxy]-3-pyridyl]methanone;
[6-(3-cyclopropyl-1,2,4-triazol-1-yl)-2-azaspiro[3.3]heptan-2-yl]−[5-fluoro-6-[(1-methylcyclopropyl)methoxy]-3-pyridyl]methanone;
[[6-(4-cyclopropylimidazol-1-yl)-2-azaspiro[3.3]heptan-2-yl]−[5-fluoro-6-[(1-methylcyclopropyl)methoxy]-3-pyridyl]methanone;
[6-(3-cyclopropyl-1,2,4-triazol-1-yl)-2-azaspiro[3.3]heptan-2-yl]−[2-[[1-(trifluoromethyl)cyclopropyl]methoxy]pyrimidin-5-yl]methanone;
[6-(3-cyclopropyl-1,2,4-triazol-1-yl)-2-azaspiro[3.3]heptan-2-yl]−[5-methyl-6-[[1-(trifluoromethyl)cyclopropyl]methoxy]pyridazin-3-yl]methanone;
[6-(3-cyclopropyl-1,2,4-triazol-1-yl)-2-azaspiro[3.3]heptan-2-yl]−[3-[4-(trifluoromethoxy)phenyl]azetidin-1-yl]methanone;
[6-(3-cyclopropyl-1,2,4-triazol-1-yl)-2-azaspiro[3.3]heptan-2-yl]−[3-[4-(1-morpholinocyclopropyl)phenyl]azetidin-1-yl]methanone;
6-(3-cyclopropyl-1,2,4-triazol-1-yl)-2-azaspiro[3.3]heptan-2-yl]−[3-[4-(1,1-difluoroethyl)phenyl]azetidin-1-yl]methanone;
[6-(3-cyclopropyl-1,2,4-triazol-1-yl)-2-azaspiro[3.3]heptan-2-yl]−[3-[3-fluoro-4-(trifluoromethoxy)phenyl]azetidin-1-yl]methanone;
[6-(3-cyclopropyl-1,2,4-triazol-1-yl)-2-azaspiro[3.3]heptan-2-yl]−[3-[4-(2,2,2-trifluoroethyl)phenyl]azetidin-1-yl]methanone;
[3-(4-cyclopropyl-2-fluoro-phenyl)azetidin-1-yl]−[6-(3-cyclopropyl-1,2,4-triazol-1-yl)-2-azaspiro[3.3]heptan-2-yl]methanone;
5-[1-[6-(3-cyclopropyl-1,2,4-triazol-1-yl)-2-azaspiro[3.3]heptane-2-carbonyl]azetidin-3-yl]-2-(trifluoromethoxy)benzonitrile;
[6-(3-cyclopropyl-1,2,4-triazol-1-yl)-2-azaspiro[3.3]heptan-2-yl]−[6-[[2-methoxy-4-(trifluoromethyl)phenyl]methyl]-2-azaspiro[3.3]heptan-2-yl]methanone;
[6-(3-cyclopropyl-1,2,4-triazol-1-yl)-2-azaspiro[3.3]heptan-2-yl]−[6-[(2,4-difluorophenyl)methyl]-2-azaspiro[3.3]heptan-2-yl]methanone;
6-(3-cyclopropyl-1,2,4-triazol-1-yl)-2-azaspiro[3.3]heptan-2-yl]−[6-[2-fluoro-4-(trifluoromethoxy)phenoxy]-2-azaspiro[3.3]heptan-2-yl]methanone;
[6-(3-cyclopropyl-1,2,4-triazol-1-yl)-2-azaspiro[3.3]heptan-2-yl]−[6-[4-(trifluoromethyl)phenoxy]-2-azaspiro[3.3]heptan-2-yl]methanone;
[3-(4-tert-butylphenyl)azetidin-1-yl]−[6-(3-cyclopropyl-1,2,4-triazol-1-yl)-2-azaspiro[3.3]heptan-2-yl]methanone;
[6-(2-chloro-4-fluoro-phenoxy)-2-azaspiro[3.3]heptan-2-yl]−[6-(3-cyclopropyl-1,2,4-triazol-1-yl)-2-azaspiro[3.3]heptan-2-yl]methanone;
(6-(3-cyclopropyl-1H-1,2,4-triazol-1-yl)-2-azaspiro[3.3]heptan-2-yl)(6-(3-fluoro-5-(trifluoromethyl)phenoxy)-2-azaspiro[3.3]heptan-2-yl)methanone;
2-[[2-[6-(3-cyclopropyl-1,2,4-triazol-1-yl)-2-azaspiro[3.3]heptane-2-carbonyl]-2-azaspiro[3.3]heptan-6-yl]oxy]-5-(trifluoromethoxy)benzonitrile;
[6-(4-cyclopropylimidazol-1-yl)-2-azaspiro[3.3]heptan-2-yl]−[3-[4-[1-(trifluoromethyl)cyclopropyl]phenyl]azetidin-1-yl]methanone;
[6-(3-cyclopropyl-1,2,4-triazol-1-yl)-2-azaspiro[3.3]heptan-2-yl]−[3-[[2-fluoro-4-(pentafluoro-λ6-sulfanyl)phenyl]methoxy]azetidin-1-yl]methanone;
(6-(3-cyclopropyl-1H-1,2,4-triazol-1-yl)-2-azaspiro[3.3]heptan-2-yl)(6-(3,4-difluorobenzyl)-2-azaspiro[3.3]heptan-2-yl)methanone;
(6-(3-cyclopropyl-1H-1,2,4-triazol-1-yl)-2-azaspiro[3.3]heptan-2-yl)(6-((6-(trifluoromethyl)pyrazin-2-yl)oxy)-2-azaspiro[3.3]heptan-2-yl)methanone;
[6-(3-cyclopropyl-1,2,4-triazol-1-yl)-2-azaspiro[3.3]heptan-2-yl]−[6-[[5-(trifluoromethyl)-2-pyridyl]oxy]-2-azaspiro[3.3]heptan-2-yl]methanone;
[3-[3-chloro-4-(trifluoromethoxy)phenyl]azetidin-1-yl]−[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[6-[4-(trifluoromethoxy)phenoxy]-2-azaspiro[3.3]heptan-2-yl]methanone;
[6-(3-cyclopropyl-1,2,4-triazol-1-yl)-2-azaspiro[3.3]heptan-2-yl]−[6-[2-(trifluoromethyl)pyrimidin-4-yl]oxy-2-azaspiro[3.3]heptan-2-yl]methanone;
(6-(3-cyclopropyl-1H-1,2,4-triazol-1-yl)-2-azaspiro[3.3]heptan-2-yl)(6-((6-(difluoromethoxy)pyridin-3-yl)oxy)-2-azaspiro[3.3]heptan-2-yl)methanone;
[6-(3-cyclopropyl-1,2,4-triazol-1-yl)-2-azaspiro[3.3]heptan-2-yl]−[6-[6-(trifluoromethyl)pyrimidin-4-yl]oxy-2-azaspiro[3.3]heptan-2-yl]methanone;
[6-(3-cyclopropyl-1,2,4-triazol-1-yl)-2-azaspiro[3.3]heptan-2-yl]−[6-[4-(trifluoromethyl)pyrimidin-2-yl]oxy-2-azaspiro[3.3]heptan-2-yl]methanone;
(6-(3-cyclopropyl-1H-1,2,4-triazol-1-yl)-2-azaspiro[3.3]heptan-2-yl)(6-(2,4-difluorophenoxy)-2-azaspiro[3.3]heptan-2-yl)methanone;
(6-(3-cyclopropyl-1H-1,2,4-triazol-1-yl)-2-azaspiro[3.3]heptan-2-yl)(6-((6-methoxypyridin-3-yl)oxy)-2-azaspiro[3.3]heptan-2-yl)methanone;
(6-(3-cyclopropyl-1H-1,2,4-triazol-1-yl)-2-azaspiro[3.3]heptan-2-yl)(6-((2-(trifluoromethyl)pyrimidin-5-yl)oxy)-2-azaspiro[3.3]heptan-2-yl)methanone;
[6-(3-cyclopropyl-1,2,4-triazol-1-yl)-2-azaspiro[3.3]heptan-2-yl]−[6-[6-(trifluoromethyl)pyridazin-3-yl]oxy-2-azaspiro[3.3]heptan-2-yl]methanone;
(6-(3-cyclopropyl-1H-1,2,4-triazol-1-yl)-2-azaspiro[3.3]heptan-2-yl)(6-((5-fluoropyridin-3-yl)oxy)-2-azaspiro[3.3]heptan-2-yl)methanone;
(4-(5-(tert-butyl)-1,2,4-oxadiazol-3-yl)phenyl)(6-(4-cyclopropyl-1H-imidazol-1-yl)-2-azaspiro[3.3]heptan-2-yl)methanone;
[6-(3-cyclopropyl-1,2,4-triazol-1-yl)-2-azaspiro[3.3]heptan-2-yl]−[6-(2,2,2-trifluoroethoxy)-2-azaspiro[3.3]heptan-2-yl]methanone;
4-[[2-[6-(3-cyclopropyl-1,2,4-triazol-1-yl)-2-azaspiro[3.3]heptane-2-carbonyl]-2-azaspiro[3.3]heptan-6-yl]oxy]-1-methyl-pyridin-2-one;
[6-(5-cyclopropyl-1,2,4-triazol-1-yl)-2-azaspiro[3.3]heptan-2-yl]−[3-[4-[1-(trifluoromethyl)cyclopropyl]phenyl]azetidin-1-yl]methanone;
[6-(3-cyclopropyl-1,2,4-triazol-1-yl)-2-azaspiro[3.3]heptan-2-yl]−[6-(3,4-difluorophenoxy)-2-azaspiro[3.3]heptan-2-yl]methanone;
[3-[3-chloro-4-(trifluoromethoxy)phenyl]azetidin-1-yl]−[6-(4-cyclopropylimidazol-1-yl)-2-azaspiro[3.3]heptan-2-yl]methanone;
[6-(3-cyclopropyl-1,2,4-triazol-1-yl)-2-azaspiro[3.3]heptan-2-yl]−[6-[5-(trifluoromethyl)pyrazin-2-yl]oxy-2-azaspiro[3.3]heptan-2-yl]methanone;
(6-(3-cyclopropyl-1H-1,2,4-triazol-1-yl)-2-azaspiro[3.3]heptan-2-yl)(3-((4-(trifluoromethoxy)benzyl)oxy)azetidin-1-yl)methanone;
(6-(3-cyclopropyl-1H-1,2,4-triazol-1-yl)-2-azaspiro[3.3]heptan-2-yl)(3-((4-(trifluoromethyl)benzyl)oxy)azetidin-1-yl)methanone;
(6-(3-cyclopropyl-1H-1,2,4-triazol-1-yl)-2-azaspiro[3.3]heptan-2-yl)(3-((2-fluoro-5-(trifluoromethyl)benzyl)oxy)azetidin-1-yl)methanone;
(6-(3-cyclopropyl-1H-1,2,4-triazol-1-yl)-2-azaspiro[3.3]heptan-2-yl)(3-((3-fluoro-4-(trifluoromethyl)benzyl)oxy)azetidin-1-yl)methanone;
(6-(3-cyclopropyl-1H-1,2,4-triazol-1-yl)-2-azaspiro[3.3]heptan-2-yl)(3-((2-fluoro-4-(trifluoromethoxy)benzyl)oxy)azetidin-1-yl)methanone;
(6-(3-cyclopropyl-1H-1,2,4-triazol-1-yl)-2-azaspiro[3.3]heptan-2-yl)(3-((3-fluoro-5-(trifluoromethyl)benzyl)oxy)azetidin-1-yl)methanone;
(3-((2-chloro-4-fluorobenzyl)oxy)azetidin-1-yl)(6-(3-cyclopropyl-1H-1,2,4-triazol-1-yl)-2-azaspiro[3.3]heptan-2-yl)methanone;
(6-(3-cyclopropyl-1H-1,2,4-triazol-1-yl)-2-azaspiro[3.3]heptan-2-yl)(3-((4-fluoro-2-(trifluoromethyl)benzyl)oxy)azetidin-1-yl)methanone;
(6-(3-cyclopropyl-1H-1,2,4-triazol-1-yl)-2-azaspiro[3.3]heptan-2-yl)(6-(2,5-difluorophenoxy)-2-azaspiro[3.3]heptan-2-yl)methanone;
(6-(3-cyclopropyl-1H-1,2,4-triazol-1-yl)-2-azaspiro[3.3]heptan-2-yl)(6-((6-(trifluoromethyl)pyridin-3-yl)oxy)-2-azaspiro[3.3]heptan-2-yl)methanone;
(6-(3-cyclopropyl-1H-1,2,4-triazol-1-yl)-2-azaspiro[3.3]heptan-2-yl)(6-((5-(trifluoromethyl)pyridin-3-yl)oxy)-2-azaspiro[3.3]heptan-2-yl)methanone;
(6-((5-chloropyridin-3-yl)oxy)-2-azaspiro[3.3]heptan-2-yl)(6-(3-cyclopropyl-1H-1,2,4-triazol-1-yl)-2-azaspiro[3.3]heptan-2-yl)methanone;
(6-((6-chloropyridin-3-yl)oxy)-2-azaspiro[3.3]heptan-2-yl)(6-(3-cyclopropyl-TH-1,2,4-triazol-1-yl)-2-azaspiro[3.3]heptan-2-yl)methanone;
[3-[2-chloro-4-(trifluoromethoxy)phenyl]azetidin-1-yl]−[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[3-[3,5-difluoro-4-(trifluoromethoxy)phenyl]azetidin-1-yl]methanone;
[6-(3-cyclopropyl-1,2,4-triazol-1-yl)-2-azaspiro[3.3]heptan-2-yl]−[3-[2-fluoro-4-(trifluoromethoxy)phenyl]azetidin-1-yl]methanone;
[3-(2-tert-butylthiazol-4-yl)azetidin-1-yl]−[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[4-[5-(2,2-dimethylpropyl)-1,2,4-oxadiazol-3-yl]phenyl]methanone;
[6-(3-cyclopropyl-1,2,4-triazol-1-yl)-2-azaspiro[3.3]heptan-2-yl]−[rac-(3aS,6aR)-5-[2-fluoro-4-(trifluoromethyl)phenoxy]-3,3a,4,5,6,6a-hexahydro-1H-cyclopenta[c]pyrrol-2-yl]methanone;
[4-(5-tert-butyl-1,2,4-oxadiazol-3-yl)phenyl]−[6-(3-cyclobutyl-1,2,4-triazol-1-yl)-2-azaspiro[3.3]heptan-2-yl]methanone;
[6-(4-cyclopropylimidazol-1-yl)-2-azaspiro[3.3]heptan-2-yl]−[rac-(3aR,6aS)-5-(2-chloro-4-fluoro-phenoxy)-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[3-[2-[4-(difluoromethoxy)phenyl]ethynyl]azetidin-1-yl]methanone;
[6-(3-cyclopropyl-1,2,4-triazol-1-yl)-2-azaspiro[3.3]heptan-2-yl]−[rac-(3aR,6aS)-5-(2-chloro-4-fluoro-phenoxy)-3,3a,4,5,6,6a-hexahydro-1H-cyclopenta[c]pyrrol-2-yl]methanone;
(3-(2-chloro-3-cyclopropylphenoxy)azetidin-1-yl)(6-(3-cyclopropyl-1H-1,2,4-triazol-1-yl)-2-azaspiro[3.3]heptan-2-yl)methanone;
(3-(4-chloro-3-cyclopropylphenoxy)azetidin-1-yl)(6-(3-cyclopropyl-1H-1,2,4-triazol-1-yl)-2-azaspiro[3.3]heptan-2-yl)methanone;
(6-(3-cyclopropyl-1H-1,2,4-triazol-1-yl)-2-azaspiro[3.3]heptan-2-yl)(3-(2-fluoro-4-(trifluoromethyl)phenoxy)azetidin-1-yl)methanone;
(3-(2-chloro-4-methylphenoxy)azetidin-1-yl)(6-(3-cyclopropyl-1H-1,2,4-triazol-1-yl)-2-azaspiro[3.3]heptan-2-yl)methanone;
(6-(3-cyclopropyl-1H-1,2,4-triazol-1-yl)-2-azaspiro[3.3]heptan-2-yl)(3-(2,4-dichlorophenoxy)azetidin-1-yl)methanone;
(3-(4-chloro-2-fluorophenoxy)azetidin-1-yl)(6-(3-cyclopropyl-1H-1,2,4-triazol-1-yl)-2-azaspiro[3.3]heptan-2-yl)methanone;
(3-((2-chloro-6-methylpyridin-3-yl)oxy)azetidin-1-yl)(6-(3-cyclopropyl-TH-1,2,4-triazol-1-yl)-2-azaspiro[3.3]heptan-2-yl)methanone;
[6-(4-cyclopropylimidazol-1-yl)-2-azaspiro[3.3]heptan-2-yl]−[3-[[4-(trifluoromethyl)phenyl]methoxy]azetidin-1-yl]methanone;
[6-(4-cyclopropylimidazol-1-yl)-2-azaspiro[3.3]heptan-2-yl]−[3-[[2-fluoro-4-(trifluoromethyl)phenyl]methoxy]azetidin-1-yl]methanone;
[6-(4-cyclopropylimidazol-1-yl)-2-azaspiro[3.3]heptan-2-yl]−[3-[[3-fluoro-4-(trifluoromethyl)phenyl]methoxy]azetidin-1-yl]methanone;
[6-(4-cyclopropylimidazol-1-yl)-2-azaspiro[3.3]heptan-2-yl]−[3-[[4-(pentafluoro-λ⁶-sulfanyl)phenyl]methoxy]azetidin-1-yl]methanone;
[6-(4-cyclopropylimidazol-1-yl)-2-azaspiro[3.3]heptan-2-yl]−[3-[[2-fluoro-4-(pentafluoro-λ⁶-sulfanyl)phenyl]methoxy]azetidin-1-yl]methanone;
[6-(4-cyclopropylimidazol-1-yl)-2-azaspiro[3.3]heptan-2-yl]−[3-[[4-methyl-3-(trifluoromethyl)phenyl]methoxy]azetidin-1-yl]methanone;
[6-(3-cyclopropyl-1,2,4-triazol-1-yl)-2-azaspiro[3.3]heptan-2-yl]−[3-[2-[2-(difluoromethyl)phenyl]ethynyl]azetidin-1-yl]methanone;
[3-[(4-chloro-2-fluoro-phenyl)methoxy]azetidin-1-yl]−[6-(4-cyclopropylimidazol-1-yl)-2-azaspiro[3.3]heptan-2-yl]methanone;
[3-[(2-chloro-4-fluoro-phenyl)methoxy]azetidin-1-yl]−[6-(4-cyclopropylimidazol-1-yl)-2-azaspiro[3.3]heptan-2-yl]methanone;
[6-(4-cyclopropylimidazol-1-yl)-2-azaspiro[3.3]heptan-2-yl]−[3-[(2,4-difluorophenyl)methoxy]azetidin-1-yl]methanone; 4-[[2-[6-(3-cyclopropyl-1,2,4-triazol-1-yl)-2-azaspiro[3.3]heptane-2-carbonyl]-2-azaspiro[3.3]heptan-6-yl]oxy]-3-fluoro-benzonitrile;
[6-(3-cyclopropyl-1,2,4-triazol-1-yl)-2-azaspiro[3.3]heptan-2-yl]−[rac-(3aS,6aR)-5-[4-(difluoromethoxy)-2-fluoro-phenoxy]-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-[4-(trifluoromethyl)phenoxy]-3,3a,4,5,6,6a-hexahydro-1H-cyclopenta[c]pyrrol-2-yl]methanone;
2-[[rac-(3aS,6aR)-2-[6-(3-cyclopropyl-1,2,4-triazol-1-yl)-2-azaspiro[3.3]heptane-2-carbonyl]-3,3a,4,5,6,6a-hexahydro-TH-cyclopenta[c]pyrrol-5-yl]oxy]-5-(trifluoromethyl)benzonitrile;
[6-(3-cyclopropyl-1,2,4-triazol-1-yl)-2-azaspiro[3.3]heptan-2-yl]−[rac-(3aS,6aR)-5-[3-chloro-4-(trifluoromethyl)phenoxy]-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-[2-methoxy-4-(trifluoromethyl)phenoxy]-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-[3-fluoro-4-(trifluoromethyl)phenoxy]-3,3a,4,5,6,6a-hexahydro-1H-cyclopenta[c]pyrrol-2-yl]methanone;
5-[[rac-(3aS,6aR)-2-[6-(3-cyclopropyl-1,2,4-triazol-1-yl)-2-azaspiro[3.3]heptane-2-carbonyl]-3,3a,4,5,6,6a-hexahydro-1H-cyclopenta[c]pyrrol-5-yl]oxy]-2-(trifluoromethyl)benzonitrile;
[6-(3-cyclopropyl-1,2,4-triazol-1-yl)-2-azaspiro[3.3]heptan-2-yl]−[rac-(3aS,6aR)-5-(3,4-difluorophenoxy)-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-[4-fluoro-3-(trifluoromethyl)phenoxy]-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-(2,4-difluorophenoxy)-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-(4-fluoro-2-methoxy-phenoxy)-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-(4-fluoro-2-methylsulfonyl-phenoxy)-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-(2,4,6-trifluorophenoxy)-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-(4-fluoro-3-methyl-phenoxy)-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-(4-fluoro-3-chloro-phenoxy)-3,3a,4,5,6,6a-hexahydro-1H-cyclopenta[c]pyrrol-2-yl]methanone; 2-fluoro-5-[[rac-(3aS,6aR)-2-[6-(3-cyclopropyl-1,2,4-triazol-1-yl)-2-azaspiro[3.3]heptane-2-carbonyl]-3,3a,4,5,6,6a-hexahydro-1H-cyclopenta[c]pyrrol-5-yl]oxy]benzonitrile;
[6-(3-cyclopropyl-1,2,4-triazol-1-yl)-2-azaspiro[3.3]heptan-2-yl]−[rac-(3aS,6aR)-5-(4,5-difluoro-2-methyl-phenoxy)-3,3a,4,5,6,6a-hexahydro-1H-cyclopenta[c]pyrrol-2-yl]methanone;
5-fluoro-2-[[rac-(3aS,6aR)-2-[6-(3-cyclopropyl-1,2,4-triazol-1-yl)-2-azaspiro[3.3]heptane-2-carbonyl]-3,3a,4,5,6,6a-hexahydro-1H-cyclopenta[c]pyrrol-5-yl]oxy]benzonitrile;
[6-(3-cyclopropyl-1,2,4-triazol-1-yl)-2-azaspiro[3.3]heptan-2-yl]−[rac-(3aS,6aR)-5-(4-fluoro-3-methylsulfonyl-phenoxy)-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-(4-fluoro-3-methoxy-phenoxy)-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-[2,4-difluoro-5-(trifluoromethyl)phenoxy]-3,3a,4,5,6,6a-hexahydro-TH-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-[4-fluoro-3-(trifluoromethoxy)phenoxy]-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-[4-(trifluoromethoxy)phenoxy]-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-[3-fluoro-4-(trifluoromethoxy)phenoxy]-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[3-[[5-(trifluoromethyl)-3-pyridyl]methoxy]azetidin-1-yl]methanone;
[6-(3-cyclopropyl-1,2,4-triazol-1-yl)-2-azaspiro[3.3]heptan-2-yl]−[6-[[1-(trifluoromethyl)cyclopropyl]methoxy]-2-azaspiro[3.3]heptan-2-yl]methanone;
[6-(3-cyclopropyl-1,2,4-triazol-1-yl)-2-azaspiro[3.3]heptan-2-yl]−[2-methyl-3-[[4-(trifluoromethyl)phenyl]methoxy]azetidin-1-yl]methanone;
[6-(4-cyclopropylimidazol-1-yl)-2-azaspiro[3.3]heptan-2-yl]−[3-[2-[2-(difluoromethyl)phenyl]ethynyl]azetidin-1-yl]methanone;
[6-(3-cyclopropyl-1,2,4-triazol-1-yl)-2-azaspiro[3.3]heptan-2-yl]−[3-[5-[1-(trifluoromethyl)cyclopropyl]-1,2,4-oxadiazol-3-yl]azetidin-1-yl]methanone;
[6-(4-cyclopropylimidazol-1-yl)-2-azaspiro[3.3]heptan-2-yl]−[3-[5-[1-(trifluoromethyl)cyclopropyl]-1,2,4-oxadiazol-3-yl]azetidin-1-yl]methanone;
[6-(4-cyclopropylimidazol-1-yl)-2-azaspiro[3.3]heptan-2-yl]−[5-methyl-6-[(1-methylcyclopropyl)methoxy]-3-pyridyl]methanone;
[6-(3-cyclopropyl-1,2,4-triazol-1-yl)-2-azaspiro[3.3]heptan-2-yl]−[5-methyl-6-[(1-methylcyclopropyl)methoxy]-3-pyridyl]methanone;
[6-(4-cyclopropylimidazol-1-yl)-2-azaspiro[3.3]heptan-2-yl]−[3-[4-(2,2,2-trifluoroethoxy)pyrazol-1-yl]azetidin-1-yl]methanone;
[6-(3-cyclopropyl-1,2,4-triazol-1-yl)-2-azaspiro[3.3]heptan-2-yl]−[3-[5-[1-(trifluoromethyl)cyclopropyl]-1,3,4-oxadiazol-2-yl]azetidin-1-yl]methanone;
[4-(5-tert-butyl-1,2,4-oxadiazol-3-yl)phenyl]−[6-(4-methylpyrazol-1-yl)-2-azaspiro[3.3]heptan-2-yl]methanone;
[6-(4-methylpyrazol-1-yl)-2-azaspiro[3.3]heptan-2-yl]−[5-methyl-6-[[1-(trifluoromethyl)cyclopropyl]methoxy]-3-pyridyl]methanone;
[4-(5-tert-butyl-1,2,4-oxadiazol-3-yl)phenyl]−[6-(3-methylpyrazol-1-yl)-2-azaspiro[3.3]heptan-2-yl]methanone;
[6-(3-cyclopropyl-1,2,4-triazol-1-yl)-2-azaspiro[3.3]heptan-2-yl]−[8-[[1-(trifluoromethyl)cyclopropyl]methoxy]-5-azaspiro[2.5]octan-5-yl]methanone;
[6-(3-cyclopropyl-1,2,4-triazol-1-yl)-2-azaspiro[3.3]heptan-2-yl]−[3,3-difluoro-4-[[1-(trifluoromethyl)cyclopropyl]methoxy]-1-piperidyl]methanone;
[6-(3-cyclopropylpyrazol-1-yl)-2-azaspiro[3.3]heptan-2-yl]−[5-methyl-6-[[1-(trifluoromethyl)cyclopropyl]methoxy]-3-pyridyl]methanone;
[6-(3-cyclopropylpyrazol-1-yl)-2-azaspiro[3.3]heptan-2-yl]−[5-methyl-6-(2,2,2-trifluoro-1,1-dimethyl-ethoxy)-3-pyridyl]methanone;
[6-(3-cyclopropyl-1,2,4-triazol-1-yl)-2-azaspiro[3.3]heptan-2-yl]−[5-(trifluoromethyl)-6-[[1-(trifluoromethyl)cyclopropyl]methoxy]-3-pyridyl]methanone;
[6-(3-cyclopropyl-1,2,4-triazol-1-yl)-2-azaspiro[3.3]heptan-2-yl]−[5-(oxetan-3-yl)-6-[[1-(trifluoromethyl)cyclopropyl]methoxy]-3-pyridyl]methanone;
[6-(3-cyclopropyl-1,2,4-triazol-1-yl)-2-azaspiro[3.3]heptan-2-yl]−[4-[[1-(trifluoromethyl)cyclopropyl]methoxymethyl]-1-bicyclo[2.2.2]octanyl]methanone;
[4-(5-tert-butyl-1,2,4-oxadiazol-3-yl)phenyl]−[6-(3-ethyl-1,2,4-triazol-1-yl)-2-azaspiro[3.3]heptan-2-yl]methanone;
[4-(5-tert-butyl-1,2,4-oxadiazol-3-yl)phenyl]−[6-(4-ethylimidazol-1-yl)-2-azaspiro[3.3]heptan-2-yl]methanone;
[4-(5-tert-butyl-1,2,4-oxadiazol-3-yl)phenyl]−[6-[4-(trifluoromethyl)imidazol-1-yl]-2-azaspiro[3.3]heptan-2-yl]methanone;
[4-(5-tert-butyl-1,2,4-oxadiazol-3-yl)phenyl]−[6-(4-chloroimidazol-1-yl)-2-azaspiro[3.3]heptan-2-yl]methanone;
1-[2-[4-(5-tert-butyl-1,2,4-oxadiazol-3-yl)benzoyl]-2-azaspiro[3.3]heptan-6-yl]imidazole-4-carbonitrile;
[6-(3-cyclopropyl-1,2,4-triazol-1-yl)-2-azaspiro[3.3]heptan-2-yl]−[4-[[1-(trifluoromethyl)cyclopropyl]methoxymethyl]norboman-1-yl]methanone;
[3-(5-tert-butyl-1,2,4-oxadiazol-3-yl)-1-bicyclo[1.1.1]pentanyl]−[6-(4-cyclopropylimidazol-1-yl)-2-azaspiro[3.3]heptan-2-yl]methanone;
[3-(5-tert-butyl-1,2,4-oxadiazol-3-yl)-1-bicyclo[1.1.1]pentanyl]−[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[3-[1-[1-(trifluoromethyl)cyclopropyl]triazol-4-yl]azetidin-1-yl]methanone;
[6-(3-cyclopropyl-1,2,4-triazol-1-yl)-2-azaspiro[3.3]heptan-2-yl]−[3-[4-(2,2,2-trifluoroethoxy)pyrazol-1-yl]azetidin-1-yl]methanone;
[6-(3-cyclopropyl-1,2,4-triazol-1-yl)-2-azaspiro[3.3]heptan-2-yl]−[3-[3-[1-(trifluoromethyl)cyclopropyl]-1,2,4-oxadiazol-5-yl]azetidin-1-yl]methanone;
[6-(3-cyclopropyl-1,2,4-triazol-1-yl)-2-azaspiro[3.3]heptan-2-yl]−[3-[1-[3-(trifluoromethyl)oxetan-3-yl]triazol-4-yl]azetidin-1-yl]methanone;
[4-(5-tert-butyl-1,3,4-oxadiazol-2-yl)phenyl]−[6-(3-chloro-1,2,4-triazol-1-yl)-2-azaspiro[3.3]heptan-2-yl]methanone;
[4-(5-tert-butyl-1,3,4-oxadiazol-2-yl)phenyl]−[6-(3-cyclopropylpyrazol-1-yl)-2-azaspiro[3.3]heptan-2-yl]methanone;
[4-(1-tert-butylpyrazol-4-yl)phenyl]−[6-(3-chloro-1,2,4-triazol-1-yl)-2-azaspiro[3.3]heptan-2-yl]methanone;
[4-(5-tert-butyl-1,2,4-oxadiazol-3-yl)phenyl]−[6-(3-cyclopropylpyrazol-1-yl)-2-azaspiro[3.3]heptan-2-yl]methanone;
[4-(5-tert-butyl-1,2,4-oxadiazol-3-yl)phenyl]−[6-(3-chloro-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[3-[[1-(trifluoromethyl)cyclopropyl]methoxymethyl]cyclobutyl]methanone;
[6-(3-cyclopropylpyrazol-1-yl)-2-azaspiro[3.3]heptan-2-yl]−[5-methyl-6-(2,2,2-trifluoro-1,1-dimethyl-ethoxy)-3-pyridyl]methanone;
[6-(3-chloro-1,2,4-triazol-1-yl)-2-azaspiro[3.3]heptan-2-yl]−[5-methyl-6-(2,2,2-trifluoro-1,1-dimethyl-ethoxy)-3-pyridyl]methanone;
[6-(3-cyclopropyl-1,2,4-triazol-1-yl)-2-azaspiro[3.3]heptan-2-yl]−[3-[[1-(trifluoromethyl)cyclopropyl]methoxy]cyclobutyl]methanone;
(6-(3-(azetidin-1-yl)-1H-1,2,4-triazol-1-yl)-2-azaspiro[3.3]heptan-2-yl)(3-((4-(trifluoromethyl)benzyl)oxy)azetidin-1-yl)methanone;
[6-(3-cyclopropyl-1,2,4-triazol-4-yl)-2-azaspiro[3.3]heptan-2-yl]−[rac-(3aS,6aR)-5-(2-chloro-4-fluoro-phenoxy)-3,3a,4,5,6,6a-hexahydro-1H-cyclopenta[c]pyrrol-2-yl]methanone;
[4-(5-tert-butyl-1,2,4-oxadiazol-3-yl)phenyl]−[6-(2-cyclopropyloxazol-5-yl)-6-hydroxy-2-azaspiro[3.3]heptan-2-yl]methanone;
[4-(5-tert-butyl-1,2,4-oxadiazol-3-yl)phenyl]−[6-(5-cyclopropylpyrazol-1-yl)-2-azaspiro[3.3]heptan-2-yl]methanone;
[3-(1-tert-butylpyrazol-4-yl)azetidin-1-yl]−[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[3-[3-methyl-4-(trifluoromethoxy)phenyl]azetidin-1-yl]methanone;
(6-(3-cyclopropyl-1H-1,2,4-triazol-1-yl)-2-azaspiro[3.3]heptan-2-yl)(3-(4-cyclopropylphenoxy)azetidin-1-yl)methanone;
[6-(3-cyclobutyl-1,2,4-triazol-1-yl)-2-azaspiro[3.3]heptan-2-yl]−[6-[(2,4-difluorophenyl)methyl]-2-azaspiro[3.3]heptan-2-yl]methanone;
(3-((3-chloro-4-cyclopropylpyridin-2-yl)oxy)azetidin-1-yl)(6-(3-cyclopropyl-1H-1,2,4-triazol-1-yl)-2-azaspiro[3.3]heptan-2-yl)methanone;
[6-[(2,4-difluorophenyl)methyl]-2-azaspiro[3.3]heptan-2-yl]−[6-(3-ethyl-1,2,4-triazol-1-yl)-2-azaspiro[3.3]heptan-2-yl]methanone;
(6-(3-cyclopropyl-1H-1,2,4-triazol-1-yl)-2-azaspiro[3.3]heptan-2-yl)(3-(3-cyclopropyl-4-(trifluoromethyl)phenoxy)azetidin-1-yl)methanone;
5-cyclopropyl-2-((1-(6-(3-cyclopropyl-1H-1,2,4-triazol-1-yl)-2-azaspiro[3.3]heptane-2-carbonyl)azetidin-3-yl)oxy)benzonitrile;
[6-(3-cyclopropyl-1,2,4-triazol-1-yl)-2-azaspiro[3.3]heptan-2-yl]−[6-[2-fluoro-4-(trifluoromethyl)benzyl]-2,6-diazaspiro[3.3]heptan-2-yl]methanone;
(6-(3-cyclopropyl-1H-1,2,4-triazol-1-yl)-2-azaspiro[3.3]heptan-2-yl)(3-(4-cyclopropyl-2-fluorophenoxy)azetidin-1-yl)methanone;
2-cyclopropyl-6-((1-(6-(3-cyclopropyl-1H-1,2,4-triazol-1-yl)-2-azaspiro[3.3]heptane-2-carbonyl)azetidin-3-yl)oxy)benzonitrile;
[6-(3-cyclopropyl-1,2,4-triazol-1-yl)-2-azaspiro[3.3]heptan-2-yl]−[6-(3-mesylbenzyl)-2-azaspiro[3.3]heptan-2-yl]methanone;
[6-(3-cyclopropyl-1,2,4-triazol-1-yl)-2-azaspiro[3.3]heptan-2-yl]−[6-[[5-(trifluoromethyl)pyrazin-2-yl]methyl]-2-azaspiro[3.3]heptan-2-yl]methanone;
methyl 3-[3-[1-[6-(3-cyclopropyl-1,2,4-triazol-1-yl)-2-azaspiro[3.3]heptane-2-carbonyl]azetidin-3-yl]oxyphenyl]-2,2-dimethylpropanoate;
N-[2-[6-(3-cyclopropyl-1,2,4-triazol-1-yl)-2-azaspiro[3.3]heptane-2-carbonyl]-2-azaspiro[3.3]heptan-6-yl]-3-(trifluoromethyl)benzenesulfonamide;
[6-(3-cyclopropyl-1,2,4-triazol-1-yl)-2-azaspiro[3.3]-2-yl]−[6-[[4-fluoro-2-(trifluoromethyl)phenyl]methyl]-2,6-diazaspiro[3.3]heptan-2-yl]methanone;
[4-[(R)-(3-cyclopropyl-1,2,4-oxadiazol-5-yl)-(4-fluorophenyl)methyl]-1-piperidyl]−[6-(3-cyclopropyl-1,2,4-triazol-1-yl)-2-azaspiro[3.3]heptan-2-yl]methanone;
(6-(3-cyclopropyl-1H-1,2,4-triazol-1-yl)-2-azaspiro[3.3]heptan-2-yl)(3-(3-cyclopropyl-2-fluorophenoxy)azetidin-1-yl)methanone;
[6-(3-cyclopropyl-1,2,4-triazol-1-yl)-2-azaspiro[3.3]heptan-2-yl]−[6-[(3,5-difluoro-2-pyridyl)methyl]-2-azaspiro[3.3]heptan-2-yl]methanone;
methyl 2-[3-[1-[6-(3-cyclopropyl-1,2,4-triazol-1-yl)-2-azaspiro[3.3]heptane-2-carbonyl]azetidin-3-yl]oxyphenyl]-2-methylpropanoate;
(6-(2-chloro-4-fluorobenzyl)-2,6-diazaspiro[3.3]heptan-2-yl)(6-(3-cyclopropyl-1H-1,2,4-triazol-1-yl)-2-azaspiro[3.3]heptan-2-yl)methanone;
(6-(3-cyclopropyl-1H-1,2,4-triazol-1-yl)-2-azaspiro[3.3]heptan-2-yl)(3-(4-(trifluoromethyl)phenoxy)azetidin-1-yl)methanone;
[6-[(2,4-difluorophenyl)methyl]-2-azaspiro[3.3]heptan-2-yl]−[6-(3-isopropyl-1,2,4-triazol-1-yl)-2-azaspiro[3.3]heptan-2-yl]methanone;
(6-(3-cyclopropyl-1H-1,2,4-triazol-1-yl)-2-azaspiro[3.3]heptan-2-yl)(3-((4-cyclopropyl-3-fluoropyridin-2-yl)oxy)azetidin-1-yl)methanone;
[6-(3-cyclopropyl-1,2,4-triazol-1-yl)-2-azaspiro[3.3]heptan-2-yl]−[6-(4-mesylbenzyl)-2-azaspiro[3.3]heptan-2-yl]methanone;
[6-(3-cyclopropyl-1,2,4-triazol-1-yl)-2-azaspiro[3.3]heptan-2-yl]−[3-[6-[3-hydroxy-3-(trifluoromethyl)azetidin-1-yl]-3-pyridyl]azetidin-1-yl]methanone;
(4-((3-cyclopropyl-1,2,4-oxadiazol-5-yl)(4-fluorophenyl)methyl)piperidin-1-yl)(6-(3-cyclopropyl-TH-1,2,4-triazol-1-yl)-2-azaspiro[3.3]heptan-2-yl)methanone;
(6-(3-cyclopropyl-1H-1,2,4-triazol-1-yl)-2-azaspiro[3.3]heptan-2-yl)(3-((6-cyclopropyl-2-fluoropyridin-3-yl)oxy)azetidin-1-yl)methanone;
[6-(3-cyclopropyl-1,2,4-triazol-1-yl)-2-azaspiro[3.3]heptan-2-yl]−[2-[3-(trifluoromethyl)phenyl]sulfonyl-2,6-diazaspiro[3.3]heptan-6-yl]methanone;
[3-[(5-cyclopropyl-2-pyridyl)oxy]azetidin-1-yl]−[6-(3-cyclopropyl-1,2,4-triazol-1-yl)-2-azaspiro[3.3]heptan-2-yl]methanone;
(6-(3-cyclopropyl-1H-1,2,4-triazol-1-yl)-2-azaspiro[3.3]heptan-2-yl)(6-((4-fluoro-2-(trifluoromethyl)phenyl)sulfonyl)-2,6-diazaspiro[3.3]heptan-2-yl)methanone;
[6-(3-cyclopropyl-1,2,4-triazol-1-yl)-2-azaspiro[3.3]heptan-2-yl]−[3-[6-[3-hydroxy-3-(trifluoromethyl)pyrrolidino]-3-pyridyl]azetidin-1-yl]methanone;
[6-(3-cyclopropyl-1,2,4-triazol-1-yl)-2-azaspiro[3.3]heptan-2-yl]−[2-(3,5-difluorophenyl)sulfonyl-2,6-diazaspiro[3.3]heptan-6-yl]methanone;
[6-(3-cyclopropyl-1,2,4-triazol-1-yl)-2-azaspiro[3.3]heptan-2-yl]−[3-[6-[(3R)-3-hydroxy-3-(trifluoromethyl)pyrrolidin-1-yl]-3-pyridyl]azetidin-1-yl]methanone;
N-[2-[6-(3-cyclopropyl-1,2,4-triazol-1-yl)-2-azaspiro[3.3]heptane-2-carbonyl]-2-azaspiro[3.3]heptan-6-yl]-2,2-dimethyl-propane-1-sulfonamide;
[6-(3-cyclopropyl-1,2,4-triazol-1-yl)-2-azaspiro[3.3]heptan-2-yl]−[3-[6-[(3S)-3-hydroxy-3-(trifluoromethyl)pyrrolidino]-3-pyridyl]azetidin-1-yl]methanone;
[6-(3-cyclopropyl-1,2,4-triazol-1-yl)-2-azaspiro[3.3]heptan-2-yl]−[6-[(5-fluoro-2-pyridyl)methyl]-2-azaspiro[3.3]heptan-2-yl]methanone;
[6-[(2,4-difluorophenyl)methyl]-2-azaspiro[3.3]heptan-2-yl]−[6-(4-methylimidazol-1-yl)-2-azaspiro[3.3]heptan-2-yl]methanone;
(6-(3-cyclopropyl-1H-1,2,4-triazol-1-yl)-2-azaspiro[3.3]heptan-2-yl)(3-(2-methoxy-3-(trifluoromethyl)phenoxy)azetidin-1-yl)methanone;
(6-(3-cyclopropyl-1H-1,2,4-triazol-1-yl)-2-azaspiro[3.3]heptan-2-yl)(3-(3-cyclopropyl-4-fluorophenoxy)azetidin-1-yl)methanone;
[6-(3-cyclopropyl-1,2,4-triazol-1-yl)-2-azaspiro[3.3]heptan-2-yl]−[6-(3,5-difluorobenzyl)-2,6-diazaspiro[3.3]heptan-2-yl]methanone;
(3-(2-chloro-3-(trifluoromethyl)phenoxy)azetidin-1-yl)(6-(3-cyclopropyl-1H-1,2,4-triazol-1-yl)-2-azaspiro[3.3]heptan-2-yl)methanone;
[6-(3-cyclopropyl-1,2,4-triazol-1-yl)-2-azaspiro[3.3]heptan-2-yl]−[2-[2-(trifluoromethyl)phenyl]sulfonyl-2,6-diazaspiro[3.3]heptan-6-yl]methanone;
(2-cyclohexylsulfonyl-2,6-diazaspiro[3.3]heptan-6-yl)-[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[2-[4-(trifluoromethyl)phenyl]sulfonyl-2,6-diazaspiro[3.3]heptan-6-yl]methanone;
[6-(3-cyclopropyl-1,2,4-triazol-1-yl)-2-azaspiro[3.3]heptan-2-yl]−[2-[3-fluoro-5-(trifluoromethyl)phenyl]sulfonyl-2,6-diazaspiro[3.3]heptan-6-yl]methanone;
[4-[(S)-(3-cyclopropyl-1,2,4-oxadiazol-5-yl)-(4-fluorophenyl)methyl]-1-piperidyl]−[6-(3-cyclopropyl-1,2,4-triazol-1-yl)-2-azaspiro[3.3]heptan-2-yl]methanone;
(6-((2-chloro-4-fluorophenyl)sulfonyl)-2,6-diazaspiro[3.3]heptan-2-yl)(6-(3-cyclopropyl-1H-1,2,4-triazol-1-yl)-2-azaspiro[3.3]heptan-2-yl)methanone;
2-[[2-[6-(3-cyclopropyl-1,2,4-triazol-1-yl)-2-azaspiro[3.3]heptane-2-carbonyl]-2,6-diazaspiro[3.3]heptan-6-yl]methyl]benzoic acid methyl ester;
[6-(3-cyclopropyl-1,2,4-triazol-1-yl)-2-azaspiro[3.3]heptan-2-yl]−[2-[4-(trifluoromethoxy)phenyl]sulfonyl-2,6-diazaspiro[3.3]heptan-6-yl]methanone;
[6-(3-cyclopropyl-1,2,4-triazol-1-yl)-2-azaspiro[3.3]heptan-2-yl]−[2-(2,4-difluorophenyl)sulfonyl-2,6-diazaspiro[3.3]heptan-6-yl]methanone;
[6-(3-cyclopropyl-1,2,4-triazol-1-yl)-2-azaspiro[3.3]heptan-2-yl]−[6-[[5-(trifluoromethyl)-2-pyridyl]methyl]-2,6-diazaspiro[3.3]heptan-2-yl]methanone;
[6-(3-cyclopropyl-1,2,4-triazol-1-yl)-2-azaspiro[3.3]heptan-2-yl]−[2-[3-(trifluoromethoxy)phenyl]sulfonyl-2,6-diazaspiro[3.3]heptan-6-yl]methanone;
[6-(3-cyclopropyl-1,2,4-triazol-1-yl)-2-azaspiro[3.3]heptan-2-yl]−[6-(2,4-difluorobenzyl)-2,6-diazaspiro[3.3]heptan-2-yl]methanone;
(3-((4-chloro-5-cyclopropylpyridin-3-yl)oxy)azetidin-1-yl)(6-(3-cyclopropyl-1H-1,2,4-triazol-1-yl)-2-azaspiro[3.3]heptan-2-yl)methanone;
[6-(3-cyclopropyl-1,2,4-triazol-1-yl)-2-azaspiro[3.3]heptan-2-yl]−[2-[4-fluoro-3-(trifluoromethyl)phenyl]sulfonyl-2,6-diazaspiro[3.3]heptan-6-yl]methanone;
[6-(3-cyclopropyl-1,2,4-triazol-1-yl)-2-azaspiro[3.3]heptan-2-yl]-(2-piperidinosulfonyl-2,6-diazaspiro[3.3]heptan-6-yl)methanone;
[6-(3-cyclopropyl-1,2,4-triazol-1-yl)-2-azaspiro[3.3]heptan-2-yl]−[7-(4-fluoro-2-mesyl-phenoxy)-2-azaspiro[3.5]nonan-2-yl]methanone;
[6-(3-cyclopropyl-1,2,4-triazol-1-yl)-2-azaspiro[3.3]heptan-2-yl]-(2-neopentylsulfonyl-2,6-diazaspiro[3.3]heptan-6-yl)methanone;
2-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]benzonitrile;
(6-(3-cyclopropyl-1H-1,2,4-triazol-1-yl)-2-azaspiro[3.3]heptan-2-yl)(3-(2-fluoro-4-methylphenoxy)azetidin-1-yl)methanone;
[6-(3-cyclopropyl-1,2,4-triazol-1-yl)-2-azaspiro[3.3]heptan-2-yl]−[2-(2,4,6-trifluorophenyl)sulfonyl-2,6-diazaspiro[3.3]heptan-6-yl]methanone;
[2-[(2-chloro-3-pyridyl)sulfonyl]-2,6-diazaspiro[3.3]heptan-6-yl]−[6-(3-cyclopropyl-1,2,4-triazol-1-yl)-2-azaspiro[3.3]heptan-2-yl]methanone;
[2-(cyclohexylmethylsulfonyl)-2,6-diazaspiro[3.3]heptan-6-yl]−[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[2-(3-methoxyphenyl)sulfonyl-2,6-diazaspiro[3.3]heptan-6-yl]methanone;
N-[[1-[6-(3-cyclopropyl-1,2,4-triazol-1-yl)-2-azaspiro[3.3]heptane-2-carbonyl]azetidin-3-yl]methyl]-3-(trifluoromethyl)benzenesulfonamide;
6-[6-(3-cyclopropyl-1,2,4-triazol-1-yl)-2-azaspiro[3.3]heptane-2-carbonyl]-N-[[1-(trifluoromethyl)cyclopropyl]methyl]-2,6-diazaspiro[3.3]heptane-2-sulfonamide;
(6-(3-cyclopropyl-1H-1,2,4-triazol-1-yl)-2-azaspiro[3.3]heptan-2-yl)(3-((6-(trifluoromethyl)pyridazin-3-yl)oxy)azetidin-1-yl)methanone;
(6-(3-cyclopropyl-TH-1,2,4-triazol-1-yl)-2-azaspiro[3.3]heptan-2-yl)(3-(4-((1,1,1-trifluoropropan-2-yl)oxy)-1H-pyrazol-1-yl)azetidin-1-yl)methanone;
(2-benzofurazan-4-ylsulfonyl-2,6-diazaspiro[3.3]heptan-6-yl)-[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[2-(2-methoxyphenyl)sulfonyl-2,6-diazaspiro[3.3]heptan-6-yl]methanone;
[6-(3-cyclopropyl-1,2,4-triazol-1-yl)-2-azaspiro[3.3]heptan-2-yl]−[3-[4-[1-(2H-tetrazol-5-yl)cyclopropyl]phenyl]azetidin-1-yl]methanone;
N-[[1-[6-(3-cyclopropyl-1,2,4-triazol-1-yl)-2-azaspiro[3.3]heptane-2-carbonyl]-4-piperidyl]methyl]-4-(trifluoromethyl)benzenesulfonamide;
(3-((6-chloro-5-cyclopropylpyridin-3-yl)oxy)azetidin-1-yl)(6-(3-cyclopropyl-1H-1,2,4-triazol-1-yl)-2-azaspiro[3.3]heptan-2-yl)methanone;
[6-(3-cyclopropyl-1,2,4-triazol-1-yl)-2-azaspiro[3.3]heptan-2-yl]−[2-[[6-(trifluoromethyl)-3-pyridyl]sulfonyl]-2,6-diazaspiro[3.3]heptan-6-yl]methanone;
6-[6-(3-cyclopropyl-1,2,4-triazol-1-yl)-2-azaspiro[3.3]heptane-2-carbonyl]-N-(4-fluorobenzyl)-2,6-diazaspiro[3.3]heptane-2-sulfonamide;
2-[[2-[6-(3-cyclopropyl-1,2,4-triazol-1-yl)-2-azaspiro[3.3]heptane-2-carbonyl]-2,6-diazaspiro[3.3]heptan-6-yl]methyl]benzenesulfonamide;
[6-(3-cyclopropyl-1,2,4-triazol-1-yl)-2-azaspiro[3.3]heptan-2-yl]−[6-[(3,5-difluoro-2-pyridyl)methyl]-2,6-diazaspiro[3.3]heptan-2-yl]methanone;
N-[[1-[6-(3-cyclopropyl-1,2,4-triazol-1-yl)-2-azaspiro[3.3]heptane-2-carbonyl]-4-piperidyl]methyl]-4-(trifluoromethoxy)benzenesulfonamide;
6-[6-(3-cyclopropyl-1,2,4-triazol-1-yl)-2-azaspiro[3.3]heptane-2-carbonyl]-N-[1-(trifluoromethyl)cyclopropyl]-2,6-diazaspiro[3.3]heptane-2-sulfonamide;
4-(1-(6-(3-cyclopropyl-1H-1,2,4-triazol-1-yl)-2-azaspiro[3.3]heptane-2-carbonyl)azetidin-3-yl)-1-(2,2,2-trifluoroethyl)pyridin-2(1H)-one;
[6-(3-cyclopropyl-1,2,4-triazol-1-yl)-2-azaspiro[3.3]heptan-2-yl]−[3-[[2-fluoro-4-(trifluoromethyl)benzyl]amino]azetidin-1-yl]methanone;
[6-(3-cyclopropyl-1,2,4-triazol-1-yl)-2-azaspiro[3.3]heptan-2-yl]−[2-[[4-(trifluoromethyl)-3-pyridyl]sulfonyl]-2,6-diazaspiro[3.3]heptan-6-yl]methanone;
2-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]benzoic acid methyl ester;
(2-benzylsulfonyl-2,6-diazaspiro[3.3]heptan-6-yl)-[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[3-(2-fluoro-4-mesyl-benzyl)oxyazetidin-1-yl]methanone;
[6-(3-cyclopropyl-1,2,4-triazol-1-yl)-2-azaspiro[3.3]heptan-2-yl]−[6-[[6-(trifluoromethyl)pyridazin-3-yl]amino]-2-azaspiro[3.3]heptan-2-yl]methanone;
[6-(3-cyclopropyl-1,2,4-triazol-1-yl)-2-azaspiro[3.3]heptan-2-yl]−[2-[[5-(trifluoromethyl)-3-pyridyl]sulfonyl]-2,6-diazaspiro[3.3]heptan-6-yl]methanone;
6-[6-(3-cyclopropyl-1,2,4-triazol-1-yl)-2-azaspiro[3.3]heptane-2-carbonyl]-N-(1-methylcyclopropyl)-2,6-diazaspiro[3.3]heptane-2-sulfonamide;
(6-(3-cyclopropyl-1H-1,2,4-triazol-1-yl)-2-azaspiro[3.3]heptan-2-yl)(3-((6-(trifluoromethyl)pyridin-3-yl)oxy)azetidin-1-yl)methanone;
4-chloro-N-[[1-[6-(3-cyclopropyl-1,2,4-triazol-1-yl)-2-azaspiro[3.3]heptane-2-carbonyl]-4-piperidyl]methyl]benzenesulfonamide;
[6-(3-cyclopropyl-1,2,4-triazol-1-yl)-2-azaspiro[3.3]heptan-2-yl]−[2-[2-(4-fluorophenyl)ethylsulfonyl]-2,6-diazaspiro[3.3]heptan-6-yl]methanone;
[6-(3-cyclopropyl-1,2,4-triazol-1-yl)-2-azaspiro[3.3]heptan-2-yl]−[2-(3,4-difluorophenyl)sulfonyl-2,6-diazaspiro[3.3]heptan-6-yl]methanone;
(6-(3-cyclopropyl-1H-1,2,4-triazol-1-yl)-2-azaspiro[3.3]heptan-2-yl)(3-((2-cyclopropylpyrimidin-4-yl)oxy)azetidin-1-yl)methanone;
(6-(6-(3-cyclopropyl-1H-1,2,4-triazol-1-yl)-2-azaspiro[3.3]heptane-2-carbonyl)-2,6-diazaspiro[3.3]heptan-2-yl)(4-fluoro-2-(trifluoromethyl)phenyl)methanone;
N-[[1-[6-(3-cyclopropyl-1,2,4-triazol-1-yl)-2-azaspiro[3.3]heptane-2-carbonyl]azetidin-3-yl]methyl]-4-(trifluoromethyl)benzenesulfonamide;
N-[2-[6-(3-cyclopropyl-1,2,4-triazol-1-yl)-2-azaspiro[3.3]heptane-2-carbonyl]-2-azaspiro[3.3]heptan-6-yl]-1-(trifluoromethyl)cyclopropanecarboxamide;
[2-[(4-chloro-3-pyridyl)sulfonyl]-2,6-diazaspiro[3.3]heptan-6-yl]−[6-(3-cyclopropyl-1,2,4-triazol-1-yl)-2-azaspiro[3.3]heptan-2-yl]methanone;
2-[3-[1-[6-(3-cyclopropyl-1,2,4-triazol-1-yl)-2-azaspiro[3.3]heptane-2-carbonyl]azetidin-3-yl]oxyphenyl]-2-methylpropanoic acid;
[3-(6-cyclopropylpyridazin-3-yl)oxyazetidin-1-yl]−[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[2-(3,5-dimethylisoxazol-4-yl)sulfonyl-2,6-diazaspiro[3.3]heptan-6-yl]methanone;
[6-(3-cyclopropyl-1,2,4-triazol-1-yl)-2-azaspiro[3.3]heptan-2-yl]−[2-(4-methoxyphenyl)sulfonyl-2,6-diazaspiro[3.3]heptan-6-yl]methanone;
(6-(3-cyclopropyl-1H-1,2,4-triazol-1-yl)-2-azaspiro[3.3]heptan-2-yl)(3-((2-(trifluoromethyl)pyrimidin-4-yl)oxy)azetidin-1-yl)methanone;
[6-(3-cyclopropyl-1,2,4-triazol-1-yl)-2-azaspiro[3.3]heptan-2-yl]-(2-pyrrolidinosulfonyl-2,6-diazaspiro[3.3]heptan-6-yl)methanone;
(6-(3-cyclopropyl-1H-1,2,4-triazol-1-yl)-2-azaspiro[3.3]heptan-2-yl)(3-((6-(trifluoromethyl)pyrimidin-4-yl)oxy)azetidin-1-yl)methanone;
[2-[(6-chloro-2-pyridyl)sulfonyl]-2,6-diazaspiro[3.3]heptan-6-yl]−[6-(3-cyclopropyl-1,2,4-triazol-1-yl)-2-azaspiro[3.3]heptan-2-yl]methanone;
3-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]benzonitrile;
[6-(3-cyclopropyl-1,2,4-triazol-1-yl)-2-azaspiro[3.3]heptan-2-yl]−[2-(1-methylcyclopropyl)sulfonyl-2,6-diazaspiro[3.3]heptan-6-yl]methanone;
(6-(3-cyclopropyl-1H-1,2,4-triazol-1-yl)-2-azaspiro[3.3]heptan-2-yl)(3-(5-(2,4-difluorophenyl)-4H-1,2,4-triazol-3-yl)azetidin-1-yl)methanone;
[6-(3-cyclopropyl-1,2,4-triazol-1-yl)-2-azaspiro[3.3]heptan-2-yl]−[2-(4-fluoro-2-methoxy-phenyl)sulfonyl-2,6-diazaspiro[3.3]heptan-6-yl]methanone;
(2S)-2-[1-[6-(3-cyclopropyl-1,2,4-triazol-1-yl)-2-azaspiro[3.3]heptane-2-carbonyl]-4-piperidyl]-2-(4-fluorophenyl)acetamide;
(6-(2-chloro-4-fluorobenzoyl)-2,6-diazaspiro[3.3]heptan-2-yl)(6-(3-cyclopropyl-1H-1,2,4-triazol-1-yl)-2-azaspiro[3.3]heptan-2-yl)methanone;
4-[1-[6-(3-cyclopropyl-1,2,4-triazol-1-yl)-2-azaspiro[3.3]heptane-2-carbonyl]azetidin-3-yl]benzenesulfonamide;
3-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]-4-fluoro-benzamide;
N-[4-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]phenyl]acetamide;
[2-(cyclopropylmethylsulfonyl)-2,6-diazaspiro[3.3]heptan-6-yl]−[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[2-[[2-(trifluoromethyl)-3-pyridyl]sulfonyl]-2,6-diazaspiro[3.3]heptan-6-yl]methanone;
4-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]benzamide;
3-[3-[1-[6-(3-cyclopropyl-1,2,4-triazol-1-yl)-2-azaspiro[3.3]heptane-2-carbonyl]azetidin-3-yl]oxyphenyl]-2,2-dimethylpropanoic acid;
[6-(3-cyclopropyl-1,2,4-triazol-1-yl)-2-azaspiro[3.3]heptan-2-yl]−[2-(5-methylisoxazol-4-yl)sulfonyl-2,6-diazaspiro[3.3]heptan-6-yl]methanone;
(2R)-2-[1-[6-(3-cyclopropyl-1,2,4-triazol-1-yl)-2-azaspiro[3.3]heptane-2-carbonyl]-4-piperidyl]-2-(4-fluorophenyl)acetamide;
N-[[1-[6-(3-cyclopropyl-1,2,4-triazol-1-yl)-2-azaspiro[3.3]heptane-2-carbonyl]azetidin-3-yl]methyl]-4-fluoro-2-(trifluoromethyl)benzenesulfonamide;
(6-(3-cyclopropyl-1H-1,2,4-triazol-1-yl)-2-azaspiro[3.3]heptan-2-yl)(3-((6-(trifluoromethyl)pyrazin-2-yl)oxy)azetidin-1-yl)methanone;
[6-(3-cyclopropyl-1,2,4-triazol-1-yl)-2-azaspiro[3.3]heptan-2-yl]-(2-triflyl-2,6-diazaspiro[3.3]heptan-6-yl)methanone;
2-[1-[6-(3-cyclopropyl-1,2,4-triazol-1-yl)-2-azaspiro[3.3]heptane-2-carbonyl]-4-piperidyl]-2-(4-fluorophenyl)acetamide;
(2S)-2-[4-[6-(3-cyclopropyl-1,2,4-triazol-1-yl)-2-azaspiro[3.3]heptane-2-carbonyl]piperazino]-2-(4-fluorophenyl)acetamide;
[3-[[4-(pentafluoro-16-sulfanyl)phenyl]methoxy]azetidin-1-yl]−[6-[4-(trifluoromethyl)imidazol-1-yl]-2-azaspiro[3.3]heptan-2-yl]methanone;
4-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]benzonitrile;
[6-(3-cyclopropyl-1,2,4-triazol-1-yl)-2-azaspiro[3.3]heptan-2-yl]−[2-[(2-methoxy-3-pyridyl)sulfonyl]-2,6-diazaspiro[3.3]heptan-6-yl]methanone;
[2-(2-aminopyrimidin-5-yl)sulfonyl-2,6-diazaspiro[3.3]heptan-6-yl]−[6-(3-cyclopropyl-1,2,4-triazol-1-yl)-2-azaspiro[3.3]heptan-2-yl]methanone;
2-[2-[6-(3-cyclopropyl-1,2,4-triazol-1-yl)-2-azaspiro[3.3]heptane-2-carbonyl]-2,6-diazaspiro[3.3]heptan-6-yl]-N-methyl-2-phenyl-acetamide;
1-(1-(6-(3-cyclopropyl-1H-1,2,4-triazol-1-yl)-2-azaspiro[3.3]heptane-2-carbonyl)azetidin-3-yl)-N-(2,2,2-trifluoroethyl)-1H-pyrazole-4-carboxamide;
2-[[2-[6-(3-cyclopropyl-1,2,4-triazol-1-yl)-2-azaspiro[3.3]heptane-2-carbonyl]-2,6-diazaspiro[3.3]heptan-6-yl]methyl]benzoic acid;
6-[6-(3-cyclopropyl-1,2,4-triazol-1-yl)-2-azaspiro[3.3]heptane-2-carbonyl]-N,N-dimethyl-2,6-diazaspiro[3.3]heptane-2-sulfonamide;
[6-(4-methylimidazol-1-yl)-2-azaspiro[3.3]heptan-2-yl]−[3-[[4-(pentafluoro-16-sulfanyl)phenyl]methoxy]azetidin-1-yl]methanone;
(6-(3-cyclopropyl-1H-1,2,4-triazol-1-yl)-2-azaspiro[3.3]heptan-2-yl)(3-((4-(trifluoromethyl)pyrimidin-2-yl)oxy)azetidin-1-yl)methanone;
[6-(3-cyclopropyl-1,2,4-triazol-1-yl)-2-azaspiro[3.3]heptan-2-yl]−[2-(3-pyridylsulfonyl)-2,6-diazaspiro[3.3]heptan-6-yl]methanone;
[6-(3-cyclopropyl-1,2,4-triazol-1-yl)-2-azaspiro[3.3]heptan-2-yl]-(2-morpholinosulfonyl-2,6-diazaspiro[3.3]heptan-6-yl)methanone;
3-[[1-[6-(3-cyclopropyl-1,2,4-triazol-1-yl)-2-azaspiro[3.3]heptane-2-carbonyl]-4-piperidyl]methyl]-4-fluoro-benzenesulfonamide;
[6-(3-cyclopropyl-1,2,4-triazol-1-yl)-2-azaspiro[3.3]heptan-2-yl]-(2-propylsulfonyl-2,6-diazaspiro[3.3]heptan-6-yl)methanone;
N-[1-[6-(3-cyclopropyl-1,2,4-triazol-1-yl)-2-azaspiro[3.3]heptane-2-carbonyl]-4-piperidyl]-4-(trifluoromethyl)benzenesulfonamide;
[6-(3-cyclopropyl-1,2,4-triazol-1-yl)-2-azaspiro[3.3]heptan-2-yl]−[3-[4-(trifluoromethyl)pyridazin-3-yl]oxyazetidin-1-yl]methanone;
2-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]benzamide;
4-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]benzoic acid;
N-[[1-[6-(3-cyclopropyl-1,2,4-triazol-1-yl)-2-azaspiro[3.3]heptane-2-carbonyl]-4-piperidyl]methyl]benzenesulfonamide;
(2R)-2-[4-[6-(3-cyclopropyl-1,2,4-triazol-1-yl)-2-azaspiro[3.3]heptane-2-carbonyl]piperazino]-2-(4-fluorophenyl)acetamide;
3-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]-4-fluoro-benzoic acid;
[6-[(2,4-difluorophenyl)methyl]-2-azaspiro[3.3]heptan-2-yl]−[6-(5-ethyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[2-(2,2,2-trifluoroethylsulfonyl)-2,6-diazaspiro[3.3]heptan-6-yl]methanone;
(2S)-2-[4-[6-(3-cyclopropyl-1,2,4-triazol-1-yl)-2-azaspiro[3.3]heptane-2-carbonyl]piperazino]-2-(4-fluorophenyl)-N-methyl-acetamide;
[6-(3-cyclopropyl-1,2,4-triazol-1-yl)-2-azaspiro[3.3]heptan-2-yl]−[2-(1-methylpyrazol-4-yl)sulfonyl-2,6-diazaspiro[3.3]heptan-6-yl]methanone;
2-[4-[6-(3-cyclopropyl-1,2,4-triazol-1-yl)-2-azaspiro[3.3]heptane-2-carbonyl]piperazino]-2-(4-fluorophenyl)acetamide;
(6-(3-cyclopropyl-1H-1,2,4-triazol-1-yl)-2-azaspiro[3.3]heptan-2-yl)(3-(5-(cyclopropylmethyl)-4H-1,2,4-triazol-3-yl)azetidin-1-yl)methanone;
2-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]-N-methyl-benzamide;
(2R)-2-[4-[6-(3-cyclopropyl-1,2,4-triazol-1-yl)-2-azaspiro[3.3]heptane-2-carbonyl]piperazino]-2-(4-fluorophenyl)-N-methyl-acetamide;
N-[1-[6-(3-cyclopropyl-1,2,4-triazol-1-yl)-2-azaspiro[3.3]heptane-2-carbonyl]-4-piperidyl]-4-fluoro-benzenesulfonamide;
1-[2-[6-(3-cyclopropyl-1,2,4-triazol-1-yl)-2-azaspiro[3.3]heptane-2-carbonyl]-2,6-diazaspiro[3.3]heptan-6-yl]-2,2,2-trifluoro-ethanone;
2-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]benzoic acid;
[6-(3-cyclopropyl-1,2,4-triazol-1-yl)-2-azaspiro[3.3]heptan-2-yl]−[2-(1,1-diketothietan-3-yl)sulfonyl-2,6-diazaspiro[3.3]heptan-6-yl]methanone;
2-[4-[6-(3-cyclopropyl-1,2,4-triazol-1-yl)-2-azaspiro[3.3]heptane-2-carbonyl]piperazino]-2-(4-fluorophenyl)-N-methyl-acetamide;
[3-[4-(4-chloro-2-methylsulfonyl-phenyl)phenyl]azetidin-1-yl]−[6-(triazol-2-yl)-2-azaspiro[3.3]heptan-2-yl]methanone;
[3-[4-(4-chloro-2-methylsulfonyl-phenyl)phenyl]azetidin-1-yl]−[6-(triazol-1-yl)-2-azaspiro[3.3]heptan-2-yl]methanone;
[6-[(2,4-difluorophenyl)methyl]-2-azaspiro[3.3]heptan-2-yl]−[6-(4-fluorophenyl)-2-azaspiro[3.3]heptan-2-yl]methanone;
[3-[4-(4-chloro-2-methylsulfonyl-phenyl)phenyl]azetidin-1-yl]−[6-[2-(trifluoromethyl)pyrimidin-5-yl]-2-azaspiro[3.3]heptan-2-yl]methanone;
[6-[(2,4-difluorophenyl)methyl]-2-azaspiro[3.3]heptan-2-yl]−[6-[4-(trifluoromethylsulfonyl)phenyl]-2-azaspiro[3.3]heptan-2-yl]methanone;
[6-[(2,4-difluorophenyl)methyl]-2-azaspiro[3.3]heptan-2-yl]−[6-(4-methylsulfonylphenyl)-2-azaspiro[3.3]heptan-2-yl]methanone;
[6-[(2,4-difluorophenyl)methyl]-2-azaspiro[3.3]heptan-2-yl]−[6-[2-methylsulfonyl-4-(trifluoromethyl)phenyl]-2-azaspiro[3.3]heptan-2-yl]methanone;
[6-[(5-chloro-2-pyridyl)methyl]-2-azaspiro[3.3]heptan-2-yl]−[6-(5-fluoro-3-pyridyl)-2,6-diazaspiro[3.3]heptan-2-yl]methanone;
[3-[4-[3-(2,2-dimethylpropyl)triazol-4-yl]phenyl]azetidin-1-yl]−[6-(5-fluoro-3-pyridyl)-2,6-diazaspiro[3.3]heptan-2-yl]methanone;
[3-[4-(4-chloro-2-methylsulfonyl-phenyl)phenyl]azetidin-1-yl]−[6-(5-fluoro-3-pyridyl)-2,6-diazaspiro[3.3]heptan-2-yl]methanone;
[6-[3-(1-hydroxycyclopropyl)-1,2,4-triazol-1-yl]-2-azaspiro[3.3]heptan-2-yl]−[(6S)-6-[(3,5-difluoro-2-pyridyl)methyl]-2-azaspiro[3.4]octan-2-yl]methanone;
[6-[3-(1-hydroxycyclopropyl)-1,2,4-triazol-1-yl]-2-azaspiro[3.3]heptan-2-yl]−[(6R)-6-[(3,5-difluoro-2-pyridyl)methyl]-2-azaspiro[3.4]octan-2-yl]methanone;
[6-[[4-(trifluoromethylsulfonyl)phenyl]methyl]-2,6-diazaspiro[3.3]heptan-2-yl]−[6-[3-(trifluoromethyl)-1,2,4-triazol-1-yl]-2-azaspiro[3.3]heptan-2-yl]methanone;
[6-[[4-fluoro-2-(methylsulfonimidoyl)phenyl]methyl]-2-azaspiro[3.3]heptan-2-yl]−[6-[3-(1-hydroxycyclopropyl)-1,2,4-triazol-1-yl]-2-azaspiro[3.3]heptan-2-yl]methanone;
[3-[3-(5-cyclopropyl-3-methyl-pyrazol-1-yl)-1-bicyclo[1.1.1]pentanyl]azetidin-1-yl]−[6-[3-(1-hydroxycyclopropyl)-1,2,4-triazol-1-yl]-2-azaspiro[3.3]heptan-2-yl]methanone;
[3-[3-(5-cyclopropyl-3-methyl-pyrazol-1-yl)-1-bicyclo[1.1.1]pentanyl]azetidin-1-yl]−[6-[3-(1-hydroxycyclopropyl)-1,2,4-triazol-1-yl]-2-azaspiro[3.3]heptan-2-yl]methanone;
[3-[3-(3,5-dimethylpyrazol-1-yl)-1-bicyclo[1.1.1]pentanyl]azetidin-1-yl]−[6-[3-(1-hydroxycyclopropyl)-1,2,4-triazol-1-yl]-2-azaspiro[3.3]heptan-2-yl]methanone;
[6-(3-cyclopropyl-1,2,4-triazol-1-yl)-2-azaspiro[3.3]heptan-2-yl]−[6-(1H-pyrazolo[4,3-b]pyridin-5-ylmethyl)-2-azaspiro[3.3]heptan-2-yl]methanone;
[6-[(2,4-difluorophenyl)methyl]-2-azaspiro[3.3]heptan-2-yl]−[6-[6-(trifluoromethyl)-3-pyridyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[[3-methylsulfonyl-5-(trifluoromethyl)phenyl]methyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[(6S)-6-[[3-(trifluoromethylsulfonyl)phenyl]methyl]-2-azaspiro[3.4]octan-2-yl]methanone;
[6-[3-(1-hydroxycyclopropyl)-1,2,4-triazol-1-yl]-2-azaspiro[3.3]heptan-2-yl]−[(6R)-6-[[3-(trifluoromethylsulfonyl)phenyl]methyl]-2-azaspiro[3.4]octan-2-yl]methanone;
[6-[3-(1-hydroxycyclopropyl)-1,2,4-triazol-1-yl]-2-azaspiro[3.3]heptan-2-yl]−[(6S)-6-[[4-(trifluoromethylsulfonyl)phenyl]methyl]-2-azaspiro[3.4]octan-2-yl]methanone;
[6-[3-(1-hydroxycyclopropyl)-1,2,4-triazol-1-yl]-2-azaspiro[3.3]heptan-2-yl]−[(6R)-6-[[4-(trifluoromethylsulfonyl)phenyl]methyl]-2-azaspiro[3.4]octan-2-yl]methanone;
[6-[(4-cyclopropylsulfonylphenyl)methyl]-2-azaspiro[3.3]heptan-2-yl]−[6-[3-(1-hydroxycyclopropyl)-1,2,4-triazol-1-yl]-2-azaspiro[3.3]heptan-2-yl]methanone;
5-[[2-[6-[3-(1-hydroxycyclopropyl)-1,2,4-triazol-1-yl]-2-azaspiro[3.3]heptane-2-carbonyl]-2-azaspiro[3.3]heptan-6-yl]methyl]-2-(trifluoromethyl)benzonitrile;
[3-[4-(4-chloro-2-methylsulfonyl-phenyl)phenyl]azetidin-1-yl]−[6-(5-fluoro-3-pyridyl)-2-azaspiro[3.3]heptan-2-yl]methanone;
[6-[(5-chloro-3-fluoro-2-pyridyl)methyl]-2-azaspiro[3.3]heptan-2-yl]−[6-[3-(1-hydroxycyclopropyl)-1,2,4-triazol-1-yl]-2-azaspiro[3.3]heptan-2-yl]methanone;
4-[[2-[6-[3-(1-hydroxycyclopropyl)-1,2,4-triazol-1-yl]-2-azaspiro[3.3]heptane-2-carbonyl]-2-azaspiro[3.3]heptan-6-yl]methyl]-2-(trifluoromethyl)benzonitrile;
[6-[3-(1-hydroxycyclopropyl)-1,2,4-triazol-1-yl]-2-azaspiro[3.3]heptan-2-yl]−[7-[[4-(trifluoromethylsulfonyl)phenyl]methyl]-2,7-diazaspiro[3.4]octan-2-yl]methanone;
3-[[2-[6-[3-(1-hydroxycyclopropyl)-1,2,4-triazol-1-yl]-2-azaspiro[3.3]heptane-2-carbonyl]-2-azaspiro[3.3]heptan-6-yl]methyl]-5-(trifluoromethyl)benzonitrile;
[3-[4-(4-chloro-2-methylsulfonyl-phenyl)phenyl]azetidin-1-yl]−[6-[6-(trifluoromethyl)-3-pyridyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[3-[6-[[1-(trifluoromethyl)cyclopropyl]methylamino]-3-pyridyl]azetidin-1-yl]methanone;
[6-[3-(1-hydroxycyclopropyl)-1,2,4-triazol-1-yl]-2-azaspiro[3.3]heptan-2-yl]−[3-[6-[(3R)-3-(trifluoromethyl)pyrrolidin-1-yl]-3-pyridyl]azetidin-1-yl]methanone;
[6-[3-(1-hydroxycyclopropyl)-1,2,4-triazol-1-yl]-2-azaspiro[3.3]heptan-2-yl]−[6-[[3-(methylsulfonimidoyl)phenyl]methyl]-2-azaspiro[3.3]heptan-2-yl]methanone;
[6-[(4-dimethylphosphorylphenyl)methyl]-2-azaspiro[3.3]heptan-2-yl]−[6-[3-(trifluoromethyl)-1,2,4-triazol-1-yl]-2-azaspiro[3.3]heptan-2-yl]methanone;
[6-(3-cyclopropyl-1,2,4-triazol-1-yl)-2-azaspiro[3.3]heptan-2-yl]−[6-[(4-dimethylphosphorylphenyl)methyl]-2-azaspiro[3.3]heptan-2-yl]methanone;
[6-[(5-dimethylphosphoryl-2-pyridyl)methyl]-2-azaspiro[3.3]heptan-2-yl]−[6-[3-(trifluoromethyl)-1,2,4-triazol-1-yl]-2-azaspiro[3.3]heptan-2-yl]methanone;
[6-(3-cyclopropyl-1,2,4-triazol-1-yl)-2-azaspiro[3.3]heptan-2-yl]−[6-[(5-dimethylphosphoryl-2-pyridyl)methyl]-2-azaspiro[3.3]heptan-2-yl]methanone;
[6-[(4-dimethylphosphorylphenyl)methyl]-2,6-diazaspiro[3.3]heptan-2-yl]−[6-[3-(trifluoromethyl)-1,2,4-triazol-1-yl]-2-azaspiro[3.3]heptan-2-yl]methanone;
[6-[(2,4-difluorophenyl)methyl]-2-azaspiro[3.3]heptan-2-yl]−[6-(5-fluoro-3-pyridyl)-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[[6-(trifluoromethoxy)-3-pyridyl]methyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[3-[3-[[6-(trifluoromethyl)-3-pyridyl]methyl]-1-bicyclo[1.1.1]pentanyl]azetidin-1-yl]methanone;
[6-[3-(1-hydroxycyclopropyl)-1,2,4-triazol-1-yl]-2-azaspiro[3.3]heptan-2-yl]−[6-(1H-pyrazolo[4,3-b]pyridin-5-ylmethyl)-2-azaspiro[3.3]heptan-2-yl]methanone;
[6-(3-cyclopropyl-1,2,4-triazol-1-yl)-2-azaspiro[3.3]heptan-2-yl]−[3-[3-[(5-cyclopropyl-4H-1,2,4-triazol-3-yl)methyl]-1-bicyclo[1.1.1]pentanyl]azetidin-1-yl]methanone;
[6-[3-(1-hydroxycyclopropyl)-1,2,4-triazol-1-yl]-2-azaspiro[3.3]heptan-2-yl]−[6-[[4-methylsulfonyl-3-(trifluoromethyl)phenyl]methyl]-2-azaspiro[3.3]heptan-2-yl]methanone;
[3-[6-(4-chloro-2-methylsulfonyl-phenyl)-3-pyridyl]azetidin-1-yl]−[6-[6-(trifluoromethyl)-3-pyridyl]-2-azaspiro[3.3]heptan-2-yl]methanone;
5-[[(6S)-2-[6-[3-(1-hydroxycyclopropyl)-1,2,4-triazol-1-yl]-2-azaspiro[3.3]heptane-2-carbonyl]-2-azaspiro[3.4]octan-6-yl]oxy]-2-(trifluoromethyl)pyridine-4-carbonitrile;
[6-[(3,5-difluoro-2-pyridyl)methyl]-2-azaspiro[3.4]octan-2-yl]−[6-[3-(1-hydroxycyclopropyl)-1,2,4-triazol-1-yl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[[5-(trifluoromethyl)-2-pyridyl]methyl]-2-azaspiro[3.4]octan-2-yl]methanone;
[6-[(5-fluoro-2-pyridyl)methyl]-2-azaspiro[3.3]heptan-2-yl]−[6-[3-(1-hydroxycyclopropyl)-1,2,4-triazol-1-yl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[7-[[5-(trifluoromethyl)-2-pyridyl]methyl]-2,7-diazaspiro[3.5]nonan-2-yl]methanone;
[6-[3-(1-hydroxycyclopropyl)-1,2,4-triazol-1-yl]-2-azaspiro[3.3]heptan-2-yl]−[7-[[6-(trifluoromethyl)-3-pyridyl]methyl]-2,7-diazaspiro[3.5]nonan-2-yl]methanone;
[6-[3-(1-hydroxycyclopropyl)-1,2,4-triazol-1-yl]-2-azaspiro[3.3]heptan-2-yl]−[3-[[2-methoxy-4-(trifluoromethyl)phenyl]methylamino]azetidin-1-yl]methanone;
[6-[3-(1-hydroxycyclopropyl)-1,2,4-triazol-1-yl]-2-azaspiro[3.3]heptan-2-yl]−[3-[4-[5-[(1-methylcyclopropyl)methyl]-4H-1,2,4-triazol-3-yl]phenyl]azetidin-1-yl]methanone;
[6-[3-(1-hydroxycyclopropyl)-1,2,4-triazol-1-yl]-2-azaspiro[3.3]heptan-2-yl]−[3-[3-[[1-(trifluoromethyl)cyclopropyl]methylamino]-1-bicyclo[1.1.1]pentanyl]azetidin-1-yl]methanone;
[3-[4-(4-chloro-2-methylsulfonyl-phenyl)phenyl]azetidin-1-yl]−[6-[3-(1-hydroxycyclopropyl)-1,2,4-triazol-1-yl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[3-[6-[(3S)-3-(trifluoromethyl)pyrrolidin-1-yl]-3-pyridyl]azetidin-1-yl]methanone;
[3-[[2-fluoro-4-(trifluoromethylsulfonyl)phenyl]methoxy]azetidin-1-yl]−[6-[3-(1-hydroxycyclopropyl)-1,2,4-triazol-1-yl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[3-[[4-(trifluoromethylsulfonyl)phenyl]methoxy]azetidin-1-yl]methanone;
[6-[3-(1-hydroxycyclopropyl)-1,2,4-triazol-1-yl]-2-azaspiro[3.3]heptan-2-yl]−[6-[[4-(trifluoromethyl)-2-pyridyl]methyl]-2-azaspiro[3.3]heptan-2-yl]methanone;
N-[2-[6-[3-(1-hydroxycyclopropyl)-1,2,4-triazol-1-yl]-2-azaspiro[3.3]heptane-2-carbonyl]-2-azaspiro[3.3]heptan-6-yl]-3-(trifluoromethyl)benzenesulfonamide;
[6-[(3,5-difluoro-2-pyridyl)methyl]-2-azaspiro[3.3]heptan-2-yl]−[6-[3-(1-hydroxycyclopropyl)-1,2,4-triazol-1-yl]-2-azaspiro[3.3]heptan-2-yl]methanone;
[6-[(2-fluoro-4-methylsulfonyl-phenyl)methyl]-2-azaspiro[3.3]heptan-2-yl]−[6-[3-(1-hydroxycyclopropyl)-1,2,4-triazol-1-yl]-2-azaspiro[3.3]heptan-2-yl]methanone;
[6-[(5-chloro-2-pyridyl)methyl]-2-azaspiro[3.3]heptan-2-yl]−[6-[3-(1-hydroxycyclopropyl)-1,2,4-triazol-1-yl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[[4-(methylsulfonimidoyl)phenyl]methyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[[3-(trifluoromethylsulfonimidoyl)phenyl]methyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[[3-(trifluoromethylsulfonimidoyl)phenyl]methyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[[5-(trifluoromethyl)-2-pyridyl]methyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[[6-(trifluoromethyl)pyridazin-3-yl]methyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[[2-(trifluoromethyl)pyrimidin-5-yl]methyl]-2-azaspiro[3.3]heptan-2-yl]methanone;
[6-[(3-fluoro-5-methylsulfonyl-phenyl)methyl]-2-azaspiro[3.3]heptan-2-yl]−[6-[3-(1-hydroxycyclopropyl)-1,2,4-triazol-1-yl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[[4-(trifluoromethylsulfonyl)phenyl]methyl]-2,6-diazaspiro[3.3]heptan-2-yl]methanone;
[3-[4-(4-chloro-2-methylsulfonyl-phenyl)phenyl]azetidin-1-yl]−[6-(1,2,4-triazol-1-yl)-2-azaspiro[3.3]heptan-2-yl]methanone;
[3-[(4-dimethylphosphorylphenyl)methoxy]azetidin-1-yl]−[6-[3-(trifluoromethyl)-1,2,4-triazol-1-yl]-2-azaspiro[3.3]heptan-2-yl]methanone;
[6-(3-cyclopropyl-1,2,4-triazol-1-yl)-2-azaspiro[3.3]heptan-2-yl]−[3-[(4-dimethylphosphorylphenyl)methoxy]azetidin-1-yl]methanone;
[6-(3-cyclopropyl-1,2,4-triazol-1-yl)-2-azaspiro[3.3]heptan-2-yl]−[6-[[4-fluoro-2-(methylsulfonimidoyl)phenyl]methyl]-2-azaspiro[3.3]heptan-2-yl]methanone;
[3-[4-[3-(2,2-dimethylpropyl)triazol-4-yl]phenyl]azetidin-1-yl]−[6-(1,2,4-triazol-1-yl)-2-azaspiro[3.3]heptan-2-yl]methanone;
[3-[3-[5-[1-(trifluoromethyl)cyclopropyl]-4H-1,2,4-triazol-3-yl]-1-bicyclo[1.1.1]pentanyl]azetidin-1-yl]−[6-[3-(trifluoromethyl)-1,2,4-triazol-1-yl]-2-azaspiro[3.3]heptan-2-yl]methanone;
[6-(3-cyclopropyl-1,2,4-triazol-1-yl)-2-azaspiro[3.3]heptan-2-yl]−[3-[3-[5-[1-(trifluoromethyl)cyclopropyl]-4H-1,2,4-triazol-3-yl]-1-bicyclo[1.1.1]pentanyl]azetidin-1-yl]methanone;
[3-[3-[[[1-(trifluoromethyl)cyclopropyl]amino]methyl]-1-bicyclo[1.1.1]pentanyl]azetidin-1-yl]-[6-[3-(trifluoromethyl)-1,2,4-triazol-1-yl]-2-azaspiro[3.3]heptan-2-yl]methanone;
[3-[3-[[1-(trifluoromethyl)cyclopropyl]methylamino]-1-bicyclo[1.1.1]pentanyl]azetidin-1-yl]−[6-[3-(trifluoromethyl)-1,2,4-triazol-1-yl]-2-azaspiro[3.3]heptan-2-yl]methanone;
[3-[6-[3-(trifluoromethyl)azetidin-1-yl]-3-pyridyl]azetidin-1-yl]−[6-[3-(trifluoromethyl)-1,2,4-triazol-1-yl]-2-azaspiro[3.3]heptan-2-yl]methanone;
[3-[2-[3-(trifluoromethyl)azetidin-1-yl]pyrimidin-5-yl]azetidin-1-yl]−[6-[3-(trifluoromethyl)-1,2,4-triazol-1-yl]-2-azaspiro[3.3]heptan-2-yl]methanone;
[3-[6-[[1-(trifluoromethyl)cyclopropyl]methylamino]-3-pyridyl]azetidin-1-yl]−[6-[3-(trifluoromethyl)-1,2,4-triazol-1-yl]-2-azaspiro[3.3]heptan-2-yl]methanone;
[3-[4-[5-[(1-methylcyclopropyl)methyl]-4H-1,2,4-triazol-3-yl]phenyl]azetidin-1-yl]−[6-[3-(trifluoromethyl)-1,2,4-triazol-1-yl]-2-azaspiro[3.3]heptan-2-yl]methanone;
[3-[6-[(3S)-3-(trifluoromethyl)pyrrolidin-1-yl]-3-pyridyl]azetidin-1-yl]−[6-[3-(trifluoromethyl)-1,2,4-triazol-1-yl]-2-azaspiro[3.3]heptan-2-yl]methanone;
[3-[6-[(3R)-3-(trifluoromethyl)pyrrolidin-1-yl]-3-pyridyl]azetidin-1-yl]−[6-[3-(trifluoromethyl)-1,2,4-triazol-1-yl]-2-azaspiro[3.3]heptan-2-yl]methanone;
[3-[6-[(1,1-dioxothietan-3-yl)methylamino]-3-pyridyl]azetidin-1-yl]−[6-[3-(trifluoromethyl)-1,2,4-triazol-1-yl]-2-azaspiro[3.3]heptan-2-yl]methanone;
2-[4-[1-[6-[3-(trifluoromethyl)-1,2,4-triazol-1-yl]-2-azaspiro[3.3]heptane-2-carbonyl]azetidin-3-yl]phenyl]benzamide;
[6-[[4-(methylsulfonimidoyl)phenyl]methyl]-2-azaspiro[3.3]heptan-2-yl]−[6-[3-(trifluoromethyl)-1,2,4-triazol-1-yl]-2-azaspiro[3.3]heptan-2-yl]methanone;
[6-[[3-(methylsulfonimidoyl)phenyl]methyl]-2-azaspiro[3.3]heptan-2-yl]−[6-[3-(trifluoromethyl)-1,2,4-triazol-1-yl]-2-azaspiro[3.3]heptan-2-yl]methanone;
[6-(3-cyclopropyl-1,2,4-triazol-1-yl)-2-azaspiro[3.3]heptan-2-yl]−[3-[3-(5-cyclopropyl-4H-1,2,4-triazol-3-yl)-1-bicyclo[1.1.1]pentanyl]azetidin-1-yl]methanone;
[3-[3-(5-cyclopropyl-4H-1,2,4-triazol-3-yl)-1-bicyclo[1.1.1]pentanyl]azetidin-1-yl]−[6-[3-(trifluoromethyl)-1,2,4-triazol-1-yl]-2-azaspiro[3.3]heptan-2-yl]methanone;
[6-(3-cyclopropyl-1,2,4-triazol-1-yl)-2-azaspiro[3.3]heptan-2-yl]−[7-[[6-(trifluoromethyl)-3-pyridyl]methyl]-2,7-diazaspiro[3.4]octan-2-yl]methanone;
[6-(3-cyclopropyl-1,2,4-triazol-1-yl)-2-azaspiro[3.3]heptan-2-yl]−[3-[3-[5-(2,2,2-trifluoroethyl)-1,3,4-oxadiazol-2-yl]-1-bicyclo[1.1.1]pentanyl]azetidin-1-yl]methanone;
[3-[3-(5-cyclopropyl-1,3,4-oxadiazol-2-yl)-1-bicyclo[1.1.1]pentanyl]azetidin-1-yl]−[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[7-[[5-(trifluoromethyl)pyrazin-2-yl]methyl]-2,7-diazaspiro[3.4]octan-2-yl]methanone;
[6-(3-cyclopropyl-1,2,4-triazol-1-yl)-2-azaspiro[3.3]heptan-2-yl]−[7-[[6-(trifluoromethyl)pyridazin-3-yl]methyl]-2,7-diazaspiro[3.4]octan-2-yl]methanone;
[6-(3-cyclopropyl-1,2,4-triazol-1-yl)-2-azaspiro[3.3]heptan-2-yl]−[7-[[5-(trifluoromethyl)-2-pyridyl]methyl]-2,7-diazaspiro[3.4]octan-2-yl]methanone;
[6-(3-cyclopropyl-1,2,4-triazol-1-yl)-2-azaspiro[3.3]heptan-2-yl]−[6-[[3-(methylsulfonimidoyl)phenyl]methyl]-2-azaspiro[3.3]heptan-2-yl]methanone;
[6-(3-cyclopropyl-1,2,4-triazol-1-yl)-2-azaspiro[3.3]heptan-2-yl]−[6-[[3-(methylsulfonimidoyl)phenyl]methyl]-2-azaspiro[3.3]heptan-2-yl]methanone;
[6-(3-cyclopropyl-1,2,4-triazol-1-yl)-2-azaspiro[3.3]heptan-2-yl]−[6-[[4-(methylsulfonimidoyl)phenyl]methyl]-2-azaspiro[3.3]heptan-2-yl]methanone;
[6-(3-cyclopropyl-1,2,4-triazol-1-yl)-2-azaspiro[3.3]heptan-2-yl]−[6-[[4-(methylsulfonimidoyl)phenyl]methyl]-2-azaspiro[3.3]heptan-2-yl]methanone;
[6-(3-cyclopropyl-1,2,4-triazol-1-yl)-2-azaspiro[3.3]heptan-2-yl]−[3-[3-[[[1-(trifluoromethyl)cyclopropyl]amino]methyl]-1-bicyclo[1.1.1]pentanyl]azetidin-1-yl]methanone;
[6-(3-cyclopropyl-1,2,4-triazol-1-yl)-2-azaspiro[3.3]heptan-2-yl]−[3-[3-[[1-(trifluoromethyl)cyclopropyl]methylamino]-1-bicyclo[1.1.1]pentanyl]azetidin-1-yl]methanone;
[6-(3-cyclopropyl-1,2,4-triazol-1-yl)-2-azaspiro[3.3]heptan-2-yl]−[6-[[4-(trifluoromethylsulfonimidoyl)phenyl]methyl]-2-azaspiro[3.3]heptan-2-yl]methanone;
[6-(3-cyclopropyl-1,2,4-triazol-1-yl)-2-azaspiro[3.3]heptan-2-yl]−[6-[[3-(trifluoromethylsulfonimidoyl)phenyl]methyl]-2-azaspiro[3.3]heptan-2-yl]methanone;
[6-(3-cyclopropyl-1,2,4-triazol-1-yl)-2-azaspiro[3.3]heptan-2-yl]−[3-[4-[5-[(1-methylcyclopropyl)methyl]-4H-1,2,4-triazol-3-yl]phenyl]azetidin-1-yl]methanone;
[6-(3-cyclopropyl-1,2,4-triazol-1-yl)-2-azaspiro[3.3]heptan-2-yl]−[6-[3-(trifluoromethyl)-1,2,4-triazol-1-yl]-2-azaspiro[3.3]heptan-2-yl]methanone;
[6-(3-cyclopropyl-1,2,4-triazol-1-yl)-2-azaspiro[3.3]heptan-2-yl]−[6-(5-cyclopropyl-1,2,4-triazol-1-yl)-2-azaspiro[3.3]heptan-2-yl]methanone;
[3-[[2-methoxy-4-(trifluoromethyl)phenyl]methylamino]azetidin-1-yl]−[6-[3-(trifluoromethyl)-1,2,4-triazol-1-yl]-2-azaspiro[3.3]heptan-2-yl]methanone;
[3-[6-[3-hydroxy-3-(trifluoromethyl)azetidin-1-yl]-3-pyridyl]azetidin-1-yl]−[6-[3-(trifluoromethyl)-1,2,4-triazol-1-yl]-2-azaspiro[3.3]heptan-2-yl]methanone;
[3-[6-(3-hydroxy-3-methyl-azetidin-1-yl)-3-pyridyl]azetidin-1-yl]−[6-[3-(trifluoromethyl)-1,2,4-triazol-1-yl]-2-azaspiro[3.3]heptan-2-yl]methanone;
3-(trifluoromethyl)-N-[2-[6-[3-(trifluoromethyl)-1,2,4-triazol-1-yl]-2-azaspiro[3.3]heptane-2-carbonyl]-2-azaspiro[3.3]heptan-6-yl]benzenesulfonamide;
[6-[(4-methylsulfonylphenyl)methyl]-2-azaspiro[3.3]heptan-2-yl]−[6-[3-(trifluoromethyl)-1,2,4-triazol-1-yl]-2-azaspiro[3.3]heptan-2-yl]methanone;
[6-[(3-methylsulfonylphenyl)methyl]-2-azaspiro[3.3]heptan-2-yl]−[6-[3-(trifluoromethyl)-1,2,4-triazol-1-yl]-2-azaspiro[3.3]heptan-2-yl]methanone;
[6-[(5-methylsulfonyl-3-pyridyl)methyl]-2-azaspiro[3.3]heptan-2-yl]−[6-[3-(trifluoromethyl)-1,2,4-triazol-1-yl]-2-azaspiro[3.3]heptan-2-yl]methanone;
[6-[(5-methylsulfonyl-2-pyridyl)methyl]-2-azaspiro[3.3]heptan-2-yl]−[6-[3-(trifluoromethyl)-1,2,4-triazol-1-yl]-2-azaspiro[3.3]heptan-2-yl]methanone;
[6-[(2-fluoro-4-methylsulfonyl-phenyl)methyl]-2-azaspiro[3.3]heptan-2-yl]−[6-[3-(trifluoromethyl)-1,2,4-triazol-1-yl]-2-azaspiro[3.3]heptan-2-yl]methanone;
[6-[(3-fluoro-5-methylsulfonyl-phenyl)methyl]-2-azaspiro[3.3]heptan-2-yl]−[6-[3-(trifluoromethyl)-1,2,4-triazol-1-yl]-2-azaspiro[3.3]heptan-2-yl]methanone;
[6-(3-cyclopropyl-1,2,4-triazol-1-yl)-2-azaspiro[3.3]heptan-2-yl]−[6-[[3-(methylsulfonimidoyl)phenyl]methyl]-2-azaspiro[3.3]heptan-2-yl]methanone;
[6-(3-cyclopropyl-1,2,4-triazol-1-yl)-2-azaspiro[3.3]heptan-2-yl]−[6-[[4-(methylsulfonimidoyl)phenyl]methyl]-2-azaspiro[3.3]heptan-2-yl]methanone;
[6-(3-cyclopropyl-1,2,4-triazol-1-yl)-2-azaspiro[3.3]heptan-2-yl]−[7-[[5-(trifluoromethyl)-2-pyridyl]methyl]-2,7-diazaspiro[3.5]nonan-2-yl]methanone;
[6-(3-cyclopropyl-1,2,4-triazol-1-yl)-2-azaspiro[3.3]heptan-2-yl]−[7-[[6-(trifluoromethyl)-3-pyridyl]methyl]-2,7-diazaspiro[3.5]nonan-2-yl]methanone;
[6-(3-cyclopropyl-1,2,4-triazol-1-yl)-2-azaspiro[3.3]heptan-2-yl]−[6-[[[3-(trifluoromethyl)oxetan-3-yl]amino]methyl]-2-azaspiro[3.3]heptan-2-yl]methanone;
[6-(3-cyclopropyl-1,2,4-triazol-1-yl)-2-azaspiro[3.3]heptan-2-yl]−[3-[4-[5-[1-(trifluoromethyl)cyclopropyl]-4H-1,2,4-triazol-3-yl]phenyl]azetidin-1-yl]methanone;
[6-(3-cyclopropyl-1,2,4-triazol-1-yl)-2-azaspiro[3.3]heptan-2-yl]−[3-[4-(5-cyclopropyl-TH-1,2,4-triazol-3-yl)phenyl]azetidin-1-yl]methanone;
[6-(3-cyclopropyl-1,2,4-triazol-1-yl)-2-azaspiro[3.3]heptan-2-yl]−[6-[(3-fluoro-5-methylsulfonyl-phenyl)methyl]-2-azaspiro[3.3]heptan-2-yl]methanone;
[6-(3-cyclopropyl-1,2,4-triazol-1-yl)-2-azaspiro[3.3]heptan-2-yl]−[6-[[4-(trifluoromethylsulfonyl)phenyl]methyl]-2,6-diazaspiro[3.3]heptan-2-yl]methanone;
[6-(3-cyclopropyl-1,2,4-triazol-1-yl)-2-azaspiro[3.3]heptan-2-yl]−[3-[5-[[1-(trifluoromethyl)cyclopropyl]methylamino]pyrazin-2-yl]azetidin-1-yl]methanone;
[3-[4-(5-cyclobutyl-1H-1,2,4-triazol-3-yl)phenyl]azetidin-1-yl]−[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[6-[[6-(trifluoromethyl)pyridazin-3-yl]methyl]-2,6-diazaspiro[3.3]heptan-2-yl]methanone;
[6-(3-cyclopropyl-1,2,4-triazol-1-yl)-2-azaspiro[3.3]heptan-2-yl]−[6-[[5-(trifluoromethyl)pyrazin-2-yl]methyl]-2,6-diazaspiro[3.3]heptan-2-yl]methanone;
[6-(3-cyclopropyl-1,2,4-triazol-1-yl)-2-azaspiro[3.3]heptan-2-yl]−[2-[[3-(trifluoromethyl)-1-bicyclo[1.1.1]pentanyl]sulfonyl]-2,6-diazaspiro[3.3]heptan-6-yl]methanone;
[6-(3-cyclopropyl-1,2,4-triazol-1-yl)-2-azaspiro[3.3]heptan-2-yl]−[6-[(4-fluoro-2-methylsulfonyl-phenyl)methyl]-2-azaspiro[3.3]heptan-2-yl]methanone;
[6-(3-cyclopropyl-1,2,4-triazol-1-yl)-2-azaspiro[3.3]heptan-2-yl]−[3-[6-[(3S)-3-(trifluoromethyl)pyrrolidin-1-yl]-3-pyridyl]azetidin-1-yl]methanone;
[6-(3-cyclopropyl-1,2,4-triazol-1-yl)-2-azaspiro[3.3]heptan-2-yl]−[3-[6-[[1-(trifluoromethyl)cyclopropyl]amino]-3-pyridyl]azetidin-1-yl]methanone;
[6-(3-cyclopropyl-1,2,4-triazol-1-yl)-2-azaspiro[3.3]heptan-2-yl]−[3-[5-[[1-(trifluoromethyl)cyclopropyl]methylamino]-2-pyridyl]azetidin-1-yl]methanone;
[6-(3-cyclopropyl-1,2,4-triazol-1-yl)-2-azaspiro[3.3]heptan-2-yl]−[3-[2-[3-(trifluoromethyl)azetidin-1-yl]pyrimidin-5-yl]azetidin-1-yl]methanone;
[6-(3-cyclopropyl-1,2,4-triazol-1-yl)-2-azaspiro[3.3]heptan-2-yl]−[3-[6-[(3S)-3-(trifluoromethyl)pyrrolidin-1-yl]-3-pyridyl]azetidin-1-yl]methanone;
[6-(3-cyclopropyl-1,2,4-triazol-1-yl)-2-azaspiro[3.3]heptan-2-yl]−[3-[2-[(3S)-3-(trifluoromethyl)pyrrolidin-1-yl]pyrimidin-5-yl]azetidin-1-yl]methanone;
[6-(3-cyclopropyl-1,2,4-triazol-1-yl)-2-azaspiro[3.3]heptan-2-yl]−[3-[2-[(3R)-3-(trifluoromethyl)pyrrolidin-1-yl]pyrimidin-5-yl]azetidin-1-yl]methanone;
[6-(3-cyclopropyl-1,2,4-triazol-1-yl)-2-azaspiro[3.3]heptan-2-yl]−[6-[(2-fluoro-4-methylsulfonyl-phenyl)methyl]-2-azaspiro[3.3]heptan-2-yl]methanone;
[6-(3-cyclopropyl-1,2,4-triazol-1-yl)-2-azaspiro[3.3]heptan-2-yl]−[7-[[3-(trifluoromethyl)-1-bicyclo[1.1.1]pentanyl]sulfonyl]-2,7-diazaspiro[3.5]nonan-2-yl]methanone;
[6-(3-cyclopropyl-1,2,4-triazol-1-yl)-2-azaspiro[3.3]heptan-2-yl]−[7-[(3-methylsulfonylphenyl)methyl]-2,7-diazaspiro[3.5]nonan-2-yl]methanone;
[6-(3-cyclopropyl-1,2,4-triazol-1-yl)-2-azaspiro[3.3]heptan-2-yl]−[7-[(4-methylsulfonylphenyl)methyl]-2,7-diazaspiro[3.5]nonan-2-yl]methanone;
[6-[3-(1-hydroxycyclopropyl)-1,2,4-triazol-1-yl]-2-azaspiro[3.3]heptan-2-yl]−[6-[[5-(trifluoromethyl)pyrazin-2-yl]methyl]-2-azaspiro[3.3]heptan-2-yl]methanone;
[6-(3-cyclopropyl-1,2,4-triazol-1-yl)-2-azaspiro[3.3]heptan-2-yl]−[6-[(5-methylsulfonyl-2-pyridyl)methyl]-2-azaspiro[3.3]heptan-2-yl]methanone;
[6-(3-cyclopropyl-1,2,4-triazol-1-yl)-2-azaspiro[3.3]heptan-2-yl]−[6-[(5-methylsulfonyl-3-pyridyl)methyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[(3-methylsulfonylphenyl)methyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[(4-methylsulfonylphenyl)methyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[3-[6-[[1-(trifluoromethyl)cyclopropyl]amino]-3-pyridyl]azetidin-1-yl]methanone;
(2R)-1-[4-[1-[6-(3-cyclopropyl-1,2,4-triazol-1-yl)-2-azaspiro[3.3]heptane-2-carbonyl]azetidin-3-yl]phenyl]-4,4-difluoro-piperidine-2-carboxamide;
(2R)-1-[4-[1-[6-(3-cyclopropyl-1,2,4-triazol-1-yl)-2-azaspiro[3.3]heptane-2-carbonyl]azetidin-3-yl]phenyl]-4,4-difluoro-piperidine-2-carboxamide;
[6-[3-(1-hydroxycyclopropyl)-1,2,4-triazol-1-yl]-2-azaspiro[3.3]heptan-2-yl]−[6-[[6-(trifluoromethyl)-3-pyridyl]methyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[3-[6-[3-(trifluoromethyl)azetidin-1-yl]-3-pyridyl]azetidin-1-yl]methanone;
[6-[3-(1-hydroxycyclopropyl)-1,2,4-triazol-1-yl]-2-azaspiro[3.3]heptan-2-yl]−[2-[3-(trifluoromethoxy)phenyl]sulfonyl-2,6-diazaspiro[3.3]heptan-6-yl]methanone;
[6-(3-cyclopropyl-1,2,4-triazol-1-yl)-2-azaspiro[3.3]heptan-2-yl]−[3-[[3-(trifluoromethylsulfonyl)phenyl]methoxy]azetidin-1-yl]methanone;
[6-(3-cyclopropyl-1,2,4-triazol-1-yl)-2-azaspiro[3.3]heptan-2-yl]−[3-[[4-(trifluoromethylsulfonyl)phenyl]methoxy]azetidin-1-yl]methanone;
[6-(3-cyclopropyl-1,2,4-triazol-1-yl)-2-azaspiro[3.3]heptan-2-yl]−[6-[(4-methylsulfonylphenyl)methyl]-2,6-diazaspiro[3.3]heptan-2-yl]methanone;
[6-(3-cyclopropyl-1,2,4-triazol-1-yl)-2-azaspiro[3.3]heptan-2-yl]−[6-[(3-methylsulfonylphenyl)methyl]-2,6-diazaspiro[3.3]heptan-2-yl]methanone;
[6-(3-cyclopropyl-1,2,4-triazol-1-yl)-2-azaspiro[3.3]heptan-2-yl]−[6-[(2-methylsulfonylphenyl)methyl]-2,6-diazaspiro[3.3]heptan-2-yl]methanone;
1-[4-[1-[6-(3-cyclopropyl-1,2,4-triazol-1-yl)-2-azaspiro[3.3]heptane-2-carbonyl]azetidin-3-yl]phenyl]-4,4-difluoro-piperidine-2-carboxamide;
[6-(3-cyclopropyl-1,2,4-triazol-1-yl)-2-azaspiro[3.3]heptan-2-yl]−[3-[6-(3-hydroxy-3-methyl-azetidin-1-yl)-3-pyridyl]azetidin-1-yl]methanone;
[6-(3-cyclopropyl-1,2,4-triazol-1-yl)-2-azaspiro[3.3]heptan-2-yl]−[3-[6-[(1,1-dioxothiolan-3-yl)amino]-3-pyridyl]azetidin-1-yl]methanone;
[6-(3-cyclopropyl-1,2,4-triazol-1-yl)-2-azaspiro[3.3]heptan-2-yl]−[4-(3-fluorophenoxy)-1-piperidyl]methanone;
[3-(4-cyclobutylphenyl)azetidin-1-yl]−[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[3-[2-(2-fluoro-6-methyl-phenyl)ethyl]azetidin-1-yl]methanone;
[6-(3-cyclopropyl-1,2,4-triazol-1-yl)-2-azaspiro[3.3]heptan-2-yl]−[3-[(E)-2-(3-fluorophenyl)vinyl]azetidin-1-yl]methanone; bis[6-(3-cyclopropyl-1,2,4-triazol-1-yl)-2-azaspiro[3.3]heptan-2-yl]methanone;
[7-[[6-(difluoromethoxy)-3-pyridyl]methyl]-2-azaspiro[3.5]nonan-2-yl]−[6-(5-fluoro-3-pyridyl)-2-azaspiro[3.3]heptan-2-yl]methanone;
[3-[3-cyclopropyl-4-(trifluoromethyl)phenoxy]azetidin-1-yl]−[6-(5-fluoro-3-pyridyl)-2-azaspiro[3.3]heptan-2-yl]methanone;
[3-[(2-chloro-4-fluoro-phenyl)methoxy]azetidin-1-yl]−[6-(5-fluoro-3-pyridyl)-2-azaspiro[3.3]heptan-2-yl]methanone;
[3-[[2-chloro-4-(trifluoromethyl)phenyl]methylamino]azetidin-1-yl]−[6-(5-fluoro-3-pyridyl)-2-azaspiro[3.3]heptan-2-yl]methanone;
[6-(5-fluoro-3-pyridyl)-2-azaspiro[3.3]heptan-2-yl]−[6-[[6-(trifluoromethyl)-3-pyridyl]methyl]-2-azaspiro[3.3]heptan-2-yl]methanone;
[7-[(5-fluoro-2-pyridyl)methyl]-2-azaspiro[3.5]nonan-2-yl]−[6-[6-(trifluoromethyl)-3-pyridyl]-2-azaspiro[3.3]heptan-2-yl]methanone;
[7-[(5-chloro-2-pyridyl)methyl]-2-azaspiro[3.5]nonan-2-yl]−[6-[6-(trifluoromethyl)-3-pyridyl]-2-azaspiro[3.3]heptan-2-yl]methanone;
[3-[[2-methoxy-4-(trifluoromethyl)phenyl]methylamino]azetidin-1-yl]−[6-[6-(trifluoromethyl)-3-pyridyl]-2-azaspiro[3.3]heptan-2-yl]methanone;
[6-[[5-(trifluoromethyl)pyrazin-2-yl]methyl]-2-azaspiro[3.3]heptan-2-yl]−[6-[6-(trifluoromethyl)-3-pyridyl]-2-azaspiro[3.3]heptan-2-yl]methanone;
[6-[6-(trifluoromethyl)-3-pyridyl]-2-azaspiro[3.3]heptan-2-yl]−[3-[[4-(trifluoromethylsulfonyl)phenyl]methoxy]azetidin-1-yl]methanone;
[7-[[6-(difluoromethoxy)-3-pyridyl]methyl]-2-azaspiro[3.5]nonan-2-yl]−[6-[6-(trifluoromethyl)-3-pyridyl]-2-azaspiro[3.3]heptan-2-yl]methanone; bis[6-[6-(trifluoromethyl)-3-pyridyl]-2-azaspiro[3.3]heptan-2-yl]methanone;
[3-[6-[3-(trifluoromethyl)azetidin-1-yl]-3-pyridyl]azetidin-1-yl]−[6-[6-(trifluoromethyl)-3-pyridyl]-2-azaspiro[3.3]heptan-2-yl]methanone;
[6-[6-(trifluoromethyl)-3-pyridyl]-2-azaspiro[3.3]heptan-2-yl]−[6-[[6-(trifluoromethyl)-3-pyridyl]methyl]-2-azaspiro[3.3]heptan-2-yl]methanone;
[6-[6-(trifluoromethyl)-3-pyridyl]-2-azaspiro[3.3]heptan-2-yl]−[6-[[6-(trifluoromethyl)-3-pyridyl]oxy]-2-azaspiro[3.3]heptan-2-yl]methanone;
[6-[6-(trifluoromethyl)-3-pyridyl]-2-azaspiro[3.3]heptan-2-yl]−[6-[[4-(trifluoromethylsulfonyl)phenyl]methyl]-2,6-diazaspiro[3.3]heptan-2-yl]methanone;
[3-[6-[[1-(trifluoromethyl)cyclopropyl]methylamino]-3-pyridyl]azetidin-1-yl]−[6-[6-(trifluoromethyl)-3-pyridyl]-2-azaspiro[3.3]heptan-2-yl]methanone;
[3-[3-cyclopropyl-4-(trifluoromethyl)phenoxy]azetidin-1-yl]−[6-[6-(trifluoromethyl)-3-pyridyl]-2-azaspiro[3.3]heptan-2-yl]methanone;
[6-[(3-methylsulfonylphenyl)methyl]-2-azaspiro[3.3]heptan-2-yl]−[6-[6-(trifluoromethyl)-3-pyridyl]-2-azaspiro[3.3]heptan-2-yl]methanone;
[6-[(4-methylsulfonylphenyl)methyl]-2-azaspiro[3.3]heptan-2-yl]−[6-[6-(trifluoromethyl)-3-pyridyl]-2-azaspiro[3.3]heptan-2-yl]methanone;
[3-[[2-chloro-4-(trifluoromethyl)phenyl]methylamino]azetidin-1-yl]−[6-[6-(trifluoromethyl)-3-pyridyl]-2-azaspiro[3.3]heptan-2-yl]methanone;
[6-[[4-methylsulfonyl-3-(trifluoromethyl)phenyl]methyl]-2-azaspiro[3.3]heptan-2-yl]−[6-[6-(trifluoromethyl)-3-pyridyl]-2-azaspiro[3.3]heptan-2-yl]methanone;
[3-[(2-chloro-4-fluoro-phenyl)methoxy]azetidin-1-yl]−[6-[6-(trifluoromethyl)-3-pyridyl]-2-azaspiro[3.3]heptan-2-yl]methanone;
[3-[6-(4-isopropyl-N-methyl-anilino)-3-pyridyl]azetidin-1-yl]−[6-[6-(trifluoromethyl)-3-pyridyl]-2-azaspiro[3.3]heptan-2-yl]methanone;
[6-[6-(trifluoromethyl)-3-pyridyl]-2-azaspiro[3.3]heptan-2-yl]−[6-[[6-(trifluoromethyl)-3-pyridyl]methyl]-2-azaspiro[3.4]octan-2-yl]methanone;
2-(trifluoromethyl)-5-[[2-[6-[6-(trifluoromethyl)-3-pyridyl]-2-azaspiro[3.3]heptane-2-carbonyl]-2-azaspiro[3.3]heptan-6-yl]methyl]benzonitrile;
[3-[5-(2,4-dichlorophenyl)-2-pyridyl]azetidin-1-yl]−[6-[6-(trifluoromethyl)-3-pyridyl]-2-azaspiro[3.3]heptan-2-yl]methanone;
[3-[6-(2-chloro-4-methylsulfonyl-phenyl)-3-pyridyl]azetidin-1-yl]−[6-[6-(trifluoromethyl)-3-pyridyl]-2-azaspiro[3.3]heptan-2-yl]methanone; and
[3-[5-(4-chloro-2-fluoro-phenyl)-2-pyridyl]azetidin-1-yl]−[6-[6-(trifluoromethyl)-3-pyridyl]-2-azaspiro[3.3]heptan-2-yl]methanone.

In a preferred embodiment, the present invention provides a compound of formula (I) as described herein, or a pharmaceutically acceptable salt thereof, wherein said compound of formula (I) is selected from:
(6-(3-cyclopropyl-1H-1,2,4-triazol-1-yl)-2-azaspiro[3.3]heptan-2-yl)(6-(2-methoxy-4-(trifluoromethyl)benzyl)-2-azaspiro[3.3]heptan-2-yl)methanone;
[6-(3-cyclopropyl-1,2,4-triazol-1-yl)-2-azaspiro[3.3]heptan-2-yl]−[6-[(2,4-difluorophenyl)methyl]-2-azaspiro[3.3]heptan-2-yl]methanone;
(6-(3-cyclopropyl-1H-1,2,4-triazol-1-yl)-2-azaspiro[3.3]heptan-2-yl)(6-(2-fluoro-4-(trifluoromethoxy)phenoxy)-2-azaspiro[3.3]heptan-2-yl)methanone;
(6-(3-cyclopropyl-1H-1,2,4-triazol-1-yl)-2-azaspiro[3.3]heptan-2-yl)(3-(4-(1-(trifluoromethyl)cyclopropyl)phenyl)azetidin-1-yl)methanone;
(6-(3-cyclopropyl-1H-1,2,4-triazol-1-yl)-2-azaspiro[3.3]heptan-2-yl)(3-(4-cyclopropylphenoxy)azetidin-1-yl)methanone;
[6-[(2,4-difluorophenyl)methyl]-2-azaspiro[3.3]heptan-2-yl]−[6-(3-ethyl-1,2,4-triazol-1-yl)-2-azaspiro[3.3]heptan-2-yl]methanone;
(6-(3-cyclopropyl-1H-1,2,4-triazol-1-yl)-2-azaspiro[3.3]heptan-2-yl)(6-(4-(trifluoromethyl)phenoxy)-2-azaspiro[3.3]heptan-2-yl)methanone;
(6-(2-chloro-4-fluorophenoxy)-2-azaspiro[3.3]heptan-2-yl)(6-(3-cyclopropyl-1H-1,2,4-triazol-1-yl)-2-azaspiro[3.3]heptan-2-yl)methanone;
(6-(3-cyclopropyl-1H-1,2,4-triazol-1-yl)-2-azaspiro[3.3]heptan-2-yl)(6-(3-fluoro-5-(trifluoromethyl)phenoxy)-2-azaspiro[3.3]heptan-2-yl)methanone;
[6-(3-cyclopropyl-1,2,4-triazol-1-yl)-2-azaspiro[3.3]heptan-2-yl]−[6-[[5-(trifluoromethyl)pyrazin-2-yl]methyl]-2-azaspiro[3.3]heptan-2-yl]methanone;
[6-(3-cyclopropyl-1,2,4-triazol-1-yl)-2-azaspiro[3.3]heptan-2-yl]−[3-[[2-fluoro-4-(pentafluoro-16-sulfanyl)phenyl]methoxy]azetidin-1-yl]methanone;
(6-(3-cyclopropyl-1H-1,2,4-triazol-1-yl)-2-azaspiro[3.3]heptan-2-yl)(6-(3,4-difluorobenzyl)-2-azaspiro[3.3]heptan-2-yl)methanone;
(6-(3-cyclopropyl-1H-1,2,4-triazol-1-yl)-2-azaspiro[3.3]heptan-2-yl)(6-((6-(trifluoromethyl)pyrazin-2-yl)oxy)-2-azaspiro[3.3]heptan-2-yl)methanone;
[6-(3-cyclopropyl-1,2,4-triazol-1-yl)-2-azaspiro[3.3]heptan-2-yl]−[3-[2-fluoro-4-(trifluoromethoxy)phenyl]azetidin-1-yl]methanone;
N-[2-[6-(3-cyclopropyl-1,2,4-triazol-1-yl)-2-azaspiro[3.3]heptane-2-carbonyl]-2-azaspiro[3.3]heptan-6-yl]-3-(trifluoromethyl)benzenesulfonamide;
[6-(3-cyclopropyl-1,2,4-triazol-1-yl)-2-azaspiro[3.3]-2-yl]−[6-[[4-fluoro-2-(trifluoromethyl)phenyl]methyl]-2,6-diazaspiro[3.3]heptan-2-yl]methanone;
(6-(3-cyclopropyl-1H-1,2,4-triazol-1-yl)-2-azaspiro[3.3]heptan-2-yl)(6-((5-(trifluoromethyl)pyridin-2-yl)oxy)-2-azaspiro[3.3]heptan-2-yl)methanone;
(6-(3-cyclopropyl-1H-1,2,4-triazol-1-yl)-2-azaspiro[3.3]heptan-2-yl)(3-(3-fluoro-4-(trifluoromethoxy)phenyl)azetidin-1-yl)methanone;
(6-(3-cyclopropyl-1H-1,2,4-triazol-1-yl)-2-azaspiro[3.3]heptan-2-yl)(3-(4-(trifluoromethoxy)phenyl)azetidin-1-yl)methanone;
[6-(3-cyclopropyl-1,2,4-triazol-1-yl)-2-azaspiro[3.3]heptan-2-yl]−[6-[(3,5-difluoro-2-pyridyl)methyl]-2-azaspiro[3.3]heptan-2-yl]methanone;
(6-(2-chloro-4-fluorobenzyl)-2,6-diazaspiro[3.3]heptan-2-yl)(6-(3-cyclopropyl-1H-1,2,4-triazol-1-yl)-2-azaspiro[3.3]heptan-2-yl)methanone;
(6-(3-cyclopropyl-1H-1,2,4-triazol-1-yl)-2-azaspiro[3.3]heptan-2-yl)(6-(3,4-difluorophenoxy)-2-azaspiro[3.3]heptan-2-yl)methanone;
[6-(3-cyclopropyl-1,2,4-triazol-1-yl)-2-azaspiro[3.3]heptan-2-yl]−[6-(4-mesylbenzyl)-2-azaspiro[3.3]heptan-2-yl]methanone;
[6-(3-cyclopropyl-1,2,4-triazol-1-yl)-2-azaspiro[3.3]heptan-2-yl]−[3-[6-[3-hydroxy-3-(trifluoromethyl)azetidin-1-yl]-3-pyridyl]azetidin-1-yl]methanone;
[6-(3-cyclopropyl-1,2,4-triazol-1-yl)-2-azaspiro[3.3]heptan-2-yl]−[2-[3-(trifluoromethyl)phenyl]sulfonyl-2,6-diazaspiro[3.3]heptan-6-yl]methanone;
(6-(3-cyclopropyl-1H-1,2,4-triazol-1-yl)-2-azaspiro[3.3]heptan-2-yl)(6-((4-fluoro-2-(trifluoromethyl)phenyl)sulfonyl)-2,6-diazaspiro[3.3]heptan-2-yl)methanone;
[6-(3-cyclopropyl-1,2,4-triazol-1-yl)-2-azaspiro[3.3]heptan-2-yl]−[3-[6-[(3R)-3-hydroxy-3-(trifluoromethyl)pyrrolidin-1-yl]-3-pyridyl]azetidin-1-yl]methanone;
(6-(3-cyclopropyl-1H-1,2,4-triazol-1-yl)-2-azaspiro[3.3]heptan-2-yl)(6-((2-(trifluoromethyl)pyrimidin-4-yl)oxy)-2-azaspiro[3.3]heptan-2-yl)methanone;
[6-(3-cyclopropyl-1,2,4-triazol-1-yl)-2-azaspiro[3.3]heptan-2-yl]−[3-[6-[(3S)-3-hydroxy-3-(trifluoromethyl)pyrrolidino]-3-pyridyl]azetidin-1-yl]methanone;
[6-(3-cyclopropyl-1,2,4-triazol-1-yl)-2-azaspiro[3.3]heptan-2-yl]−[6-[(5-fluoro-2-pyridyl)methyl]-2-azaspiro[3.3]heptan-2-yl]methanone;
(6-(3-cyclopropyl-1H-1,2,4-triazol-1-yl)-2-azaspiro[3.3]heptan-2-yl)(6-((5-(trifluoromethyl)pyrazin-2-yl)oxy)-2-azaspiro[3.3]heptan-2-yl)methanone;
(6-(3-cyclopropyl-1H-1,2,4-triazol-1-yl)-2-azaspiro[3.3]heptan-2-yl)(3-((3-fluoro-5-(trifluoromethyl)benzyl)oxy)azetidin-1-yl)methanone;
(6-(3-cyclopropyl-1H-1,2,4-triazol-1-yl)-2-azaspiro[3.3]heptan-2-yl)(6-((6-(trifluoromethyl)pyridin-3-yl)oxy)-2-azaspiro[3.3]heptan-2-yl)methanone;
(6-(3-cyclopropyl-1H-1,2,4-triazol-1-yl)-2-azaspiro[3.3]heptan-2-yl)(6-((6-(trifluoromethyl)pyrimidin-4-yl)oxy)-2-azaspiro[3.3]heptan-2-yl)methanone;
(6-(3-cyclopropyl-1H-1,2,4-triazol-1-yl)-2-azaspiro[3.3]heptan-2-yl)(6-(2,4-difluorophenoxy)-2-azaspiro[3.3]heptan-2-yl)methanone;
[6-(3-cyclopropyl-1,2,4-triazol-1-yl)-2-azaspiro[3.3]heptan-2-yl]−[2-[4-(trifluoromethoxy)phenyl]sulfonyl-2,6-diazaspiro[3.3]heptan-6-yl]methanone;
(4-(5-(tert-butyl)-1,2,4-oxadiazol-3-yl)phenyl)(6-(3-cyclopropyl-1H-1,2,4-triazol-1-yl)-2-azaspiro[3.3]heptan-2-yl)methanone;
[6-(3-cyclopropyl-1,2,4-triazol-1-yl)-2-azaspiro[3.3]heptan-2-yl]−[7-(4-fluoro-2-mesyl-phenoxy)-2-azaspiro[3.5]nonan-2-yl]methanone;
(6-(3-cyclopropyl-1H-1,2,4-triazol-1-yl)-2-azaspiro[3.3]heptan-2-yl)(5-methyl-6-((1-(trifluoromethyl)cyclopropyl)methoxy)pyridin-3-yl)methanone; and
(6-(3-cyclopropyl-1H-1,2,4-triazol-1-yl)-2-azaspiro[3.3]heptan-2-yl)(3-(3-((1-(trifluoromethyl)cyclopropyl)methyl)-1,2,4-oxadiazol-5-yl)azetidin-1-yl)methanone.

In a particularly preferred embodiment, the present invention provides a compound of formula (I) as described herein, or a pharmaceutically acceptable salt thereof, wherein said compound of formula (I) is selected from: [6-(3-cyclopropyl-1,2,4-triazol-1-yl)-2-azaspiro[3.3]heptan-2-yl]−[6-[(2,4-difluorophenyl)methyl]-2-azaspiro[3.3]heptan-2-yl]methanone;
(6-(3-cyclopropyl-1H-1,2,4-triazol-1-yl)-2-azaspiro[3.3]heptan-2-yl)(3-(4-(1-(trifluoromethyl)cyclopropyl)phenyl)azetidin-1-yl)methanone;
(6-(3-cyclopropyl-1H-1,2,4-triazol-1-yl)-2-azaspiro[3.3]heptan-2-yl)(6-(4-(trifluoromethyl)phenoxy)-2-azaspiro[3.3]heptan-2-yl)methanone;
[6-(3-cyclopropyl-1,2,4-triazol-1-yl)-2-azaspiro[3.3]heptan-2-yl]−[6-[[5-(trifluoromethyl)pyrazin-2-yl]methyl]-2-azaspiro[3.3]heptan-2-yl]methanone;
[6-(3-cyclopropyl-1,2,4-triazol-1-yl)-2-azaspiro[3.3]heptan-2-yl]−[6-(4-mesylbenzyl)-2-azaspiro[3.3]heptan-2-yl]methanone;
[6-(3-cyclopropyl-1,2,4-triazol-1-yl)-2-azaspiro[3.3]heptan-2-yl]−[2-[3-(trifluoromethyl)phenyl]sulfonyl-2,6-diazaspiro[3.3]heptan-6-yl]methanone;
[6-(3-cyclopropyl-1,2,4-triazol-1-yl)-2-azaspiro[3.3]heptan-2-yl]−[6-[(5-fluoro-2-pyridyl)methyl]-2-azaspiro[3.3]heptan-2-yl]methanone;
(4-(5-(tert-butyl)-1,2,4-oxadiazol-3-yl)phenyl)(6-(3-cyclopropyl-1H-1,2,4-triazol-1-yl)-2-azaspiro[3.3]heptan-2-yl)methanone; and
(6-(3-cyclopropyl-1H-1,2,4-triazol-1-yl)-2-azaspiro[3.3]heptan-2-yl)(3-(3-((1-(trifluoromethyl)cyclopropyl)methyl)-1,2,4-oxadiazol-5-yl)azetidin-1-yl)methanone.

In a particular embodiment, the present invention provides pharmaceutically acceptable salts of the compounds according to formula (I) as described herein. In a further particular embodiment, the present invention provides compounds according to formula (I) as described herein as free bases.

In some embodiments, the compounds of formula (I) are isotopically-labeled by having one or more atoms therein replaced by an atom having a different atomic mass or mass number. Such isotopically-labeled (i.e., radiolabeled) compounds of formula (I) are considered to be within the scope of this disclosure. Examples of isotopes that can be incorporated into the compounds of formula (I) include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Certain isotopically-labeled compounds of formula (I), for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of formula (I) can be enriched with 1, 2, 5, 10, 25, 50, 75, 90, 95, or 99 percent of a given isotope.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the Examples as set out below using an appropriate isotopically-labeled reagent in place of the non-labeled reagent previously employed.

Processes of Manufacturing

The preparation of compounds of formula (I) of the present invention may be carried out in sequential or convergent synthetic routes. Syntheses of the invention are shown in the following general schemes. The skills required for carrying out the reaction and purification of the resulting products are known to those persons skilled in the art. The substituents and indices used in the following description of the processes have the significance given herein, unless indicated to the contrary.

If one of the starting materials, intermediates or compounds of formula (I) contain one or more functional groups which are not stable or are reactive under the reaction conditions of one or more reaction steps, appropriate protective groups (as described e.g., in “Protective Groups in Organic Chemistry” by T. W. Greene and P. G. M. Wutts, 5th Ed., 2014, John Wiley & Sons, N.Y.) can be introduced before the critical step applying methods well known in the art. Such protective groups can be removed at a later stage of the synthesis using standard methods described in the literature.

If starting materials or intermediates contain stereogenic centers, compounds of formula (I) can be obtained as mixtures of diastereomers or enantiomers, which can be separated by methods well known in the art e.g., chiral HPLC, chiral SFC or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent. It is equally possible to separate starting materials and intermediates containing stereogenic centers to afford diastereomerically/enantiomerically enriched starting materials and intermediates. Using such diastereomerically/enantiomerically enriched starting materials and intermediates in the synthesis of compounds of formula (I) will typically lead to the respective diastereomerically/enantiomerically enriched compounds of formula (I).

A person skilled in the art will acknowledge that in the synthesis of compounds of formula (I)-insofar not desired otherwise—an “orthogonal protection group strategy” will be applied, allowing the cleavage of several protective groups one at a time each without affecting other protective groups in the molecule. The principle of orthogonal protection is well known in the art and has also been described in literature (e.g. Barany and R. B. Merrifield, J Am. Chem. Soc. 1977, 99, 7363; H. Waldmann et al., Angew. Chem. Int. Ed. Engl. 1996, 35, 2056).

A person skilled in the art will acknowledge that the sequence of reactions may be varied depending on reactivity and nature of the intermediates.

In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 2nd Edition, Richard C. Larock. John Wiley & Sons, New York, NY. 1999). It was found convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 hours to several days will usually suffice to yield the described intermediates and compounds. The reaction sequence is not limited to the one displayed in the schemes, however, depending on the starting materials and their respective reactivity, the sequence of reaction steps can be freely altered.

If starting materials or intermediates are not commercially available or their synthesis not described in literature, they can be prepared in analogy to existing procedures for close analogues or as outlined in the experimental section.

The following abbreviations are used in the present text:
AcOH=acetic acid, ACN=acetonitrile, Bn=benzyl, BINAP=(2,2′-bis(diphenylphosphino)-1,1′-binaphthyl), Boc=tert-butyloxycarbonyl, CAS RN=chemical abstracts registration number, Cbz=benzyloxycarbonyl, Cs₂CO₃=cesium carbonate, CO=carbon monoxide, CuCl=copper(I) chloride, CuCN=copper(I) cyanide, CuI=copper(I) iodide, DABCO=1,4-Diazabicyclo[2.2.2]octane;triethylenediamine, DAST=(diethylamino)sulfur trifluoride, DBU=1,8-diazabicyclo[5,4,0]undec-7-ene, DEAD=diethyl azodicarboxylate, DIAD=diisopropyl azodicarboxylate, DIBAL-H=diisobutyl aluminium hydride, DMAP=4-dimethylaminopyridine, DME=dimethoxyethane, DMEDA=N,N′-dimethylethylenediamine, DMF=N,N-dimethylformamide, DIPEA=N,N-diisopropylethylamine, dppf=1,1 bis(diphenyl phosphino)ferrocene, EDC·HCl=N-(3-dimethylaminopropyl)-N′-ethylcarbodiimide hydrochloride, EI=electron impact, ESI=electrospray ionization, EtOAc=ethyl acetate, EtOH=ethanol, h=hour(s), FA=formic acid, H₂O=water, H₂SO₄=sulfuric acid, HATU=1-[bis(dimethylamino)methylene]-1H-1,2,3-triazolo[4,5-b]pyridinium-3-oxide hexafluorophosphate, HBTU═O-benzotriazole-N,N,N′,N′-tetramethyl-uronium-hexafluoro-phosphate, HCl=hydrogen chloride, HOBt=1-hydroxy-1H-benzotriazole; HPLC=high performance liquid chromatography, iPrMgCl=isopropylmagnesium chloride, 12=iodine, IPA=2-propanol, ISP=ion spray positive (mode), ISN=ion spray negative (mode), K₂CO₃=potassium carbonate, KHCO₃=potassium bicarbonate, KI=potassium iodide, KOH=potassium hydroxide, K₃PO₄=potassium phosphate tribasic, LiAlH₄ or LAH=lithium aluminium hydride, LiHMDS=lithium bis(trimethylsilyl)amide, LiOH=lithium hydroxide, mCPBA=meta-chloroperoxybenzoic acid, MgSO₄=magnesium sulfate, min=minute(s), mL=milliliter, MPLC=medium pressure liquid chromatography, MS=mass spectrum, nBuLi=n-butyllithium, NaBH₃CN=sodium cyanoborohydride, NaH=sodium hydride, NBS=N-bromosuccinimide, NaHCO₃=sodium hydrogen carbonate, NaNO₂=sodium nitrite, NaBH(OAc)₃=sodium triacetoxyborohydride, NaOH=sodium hydroxide, Na₂CO₃=sodium carbonate, Na₂SO₄=sodium sulfate, Na₂S₂O₃=sodium thiosulfate, NEt₃=triethylamine (TEA), NH₄Cl=ammonium chloride, NMP=N-methyl-2-pyrrolidone, OAc=Acetoxy, T₃P=propylphosphonic anhydride, PE=petroleum ether, PG=protective group, Pd—C=palladium on activated carbon, PdCl₂(dppf)-CH₂Cl₂=1,1′-bis(diphenylphosphino)ferrocene-palladium(II)dichloride dichloromethane complex, Pd₂(dba)₃=tris(dibenzylideneacetone)dipalladium(O), Pd(OAc)₂=palladium(II) acetate, Pd(OH)₂=palladium hydroxide, Pd(PPh₃)₄=tetrakis(triphenylphosphine)palladium(O), PMP=1,2,2,6,6-Pentamethylpiperidine, PTSA=p-toluenesulfonic acid, R=any group, RP=reverse phase, RT=room temperature, SFC=Supercritical Fluid Chromatography, S—PHOS=2-dicyclohexylphosphino-2′,6′-dimethoxybiphenyl, TBAI=tetra butyl ammonium iodine, TEA=triethylamine, TFA=trifluoroacetic acid, THF=tetrahydrofuran, TMEDA=N,N,N′,N′-tetramethylethylenediamine, TS-TPP=triphenylphospine—polymer bound, ZnCl₂=zinc chloride, Hal=halogen, prep-TLC=preparative thin layer chromatography.

The present compounds of formula I, where ring A is an N-linked aliphatic heterocycle, can be prepared by reacting an activated intermediate of formula 2 with the nucleophilic cyclic amine by heating in a solvent such as DMF or CH₃CN in the presence of a base such as DIPEA. In some cases an alternative activated intermediate bearing a 4-nitrophenyl group instead of the 1,2,4-triazole was used. (Scheme 1)

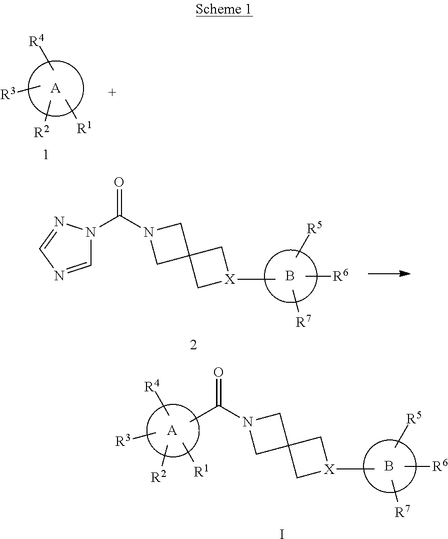

The activated intermediate 2 can be generated by reacting an amine 3 with a coupling agent such as di(1H-1,2,4-triazol-1-yl)methanone in a solvent such as CH₂Cl₂ in the presence of a base such as DIPEA (Scheme 2). The related 4-nitrophenylcarbonate intermediates can be generated in a similar process using 4-nitrophenyl carbonochloridate. Alternatively, the same strategy as in Schemes 1 and 2 may be used, but with the activated intermediate being constructed initially on the Ring A, before coupling with amine 3.

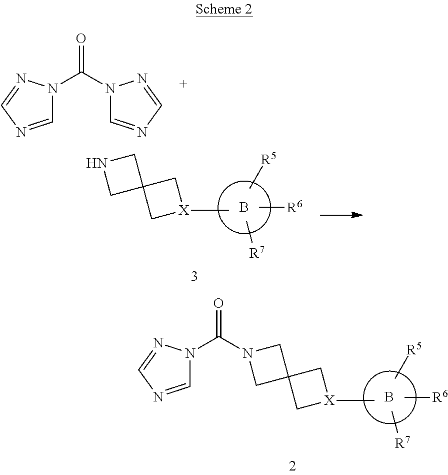

Where ring B is a N-linked aromatic heterocycle and X=CR^{B}, amine 3 can be generated by reacting the nucleophilic heteroaryl 5 with a suitably functionalized 2-azaspiro[3.3]heptane building block 4 (Y=leaving group e.g. OMs, C₁, I, Br) in the presence of a base (e.g. DIPEA, Cs₂CO₃), followed by deprotection of the protecting group using standard conditions (e.g. using TFA where PG=Boc). (Scheme 3) Typically mesylate building blocks were used (Y═OMs), which can conveniently be generated from the hydroxyl analog by reacting with MsCl in the presence of a mild base such as Et₃N.

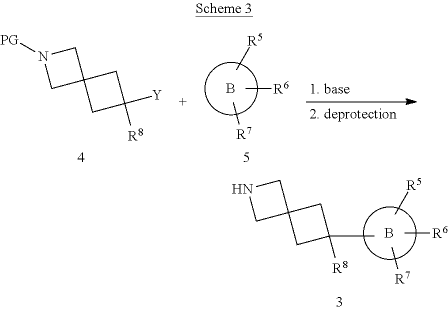

Alternatively, compounds of formula I, where ring A is an N-linked aliphatic heterocycle, can be generated by direct coupling of building blocks 1 and 3, for example using a coupling agent such as CDI or triphosgene and a base (e.g. TEA, DIPEA) (Scheme 4). Intermediates which also fall under formula I (e.g. 14, 16), can also be prepared in this manner.

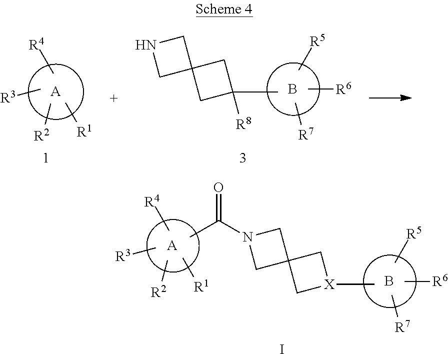

Alternatively, compounds of formula I, where ring A is C-linked, can be generated by coupling a suitable acid with the amine 3 (e.g. using a coupling agent such as HATU or T3P, and a base such as TEA or DIPEA). (Scheme 5). Intermediates which also fall under formula I (e.g. 12), can also be prepared in this manner.

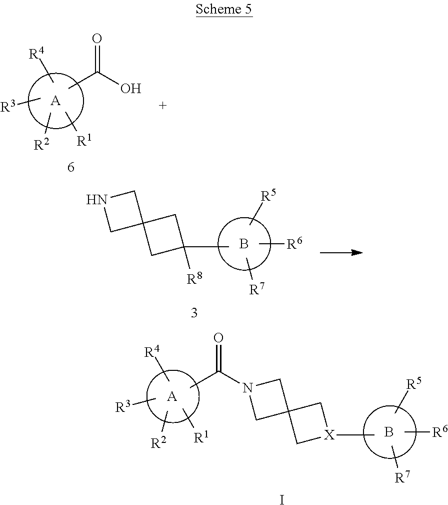

Alternatively, compounds of formula I, where X is CR⁸ and ring B is a N-linked aromatic heterocycle, can be generated by reacting a nucleophilic heteroaryl 5, with a suitably functionalized intermediate 7 (Y=leaving group, e.g. OMs) in the presence of a base (e.g. Cs₂CO₃, NaOtBu). (Scheme 6). The intermediate 7 can be generated by coupling a suitable hydroxylated building block with Ring A as described in Schemes 1 or 4, to generate intermediate 8, followed by conversion of the hydroxyl group into a suitable leaving group (e.g. by mesylation in the presence of MsCl and a base). Alternatively the hydroxylated intermediate 8 may be converted directly into a compound of formula I using Mitsunobu-type conditions (e.g. PS—PPh₃, DIAD in THF) and the nucleophilic heteroaryl 5.

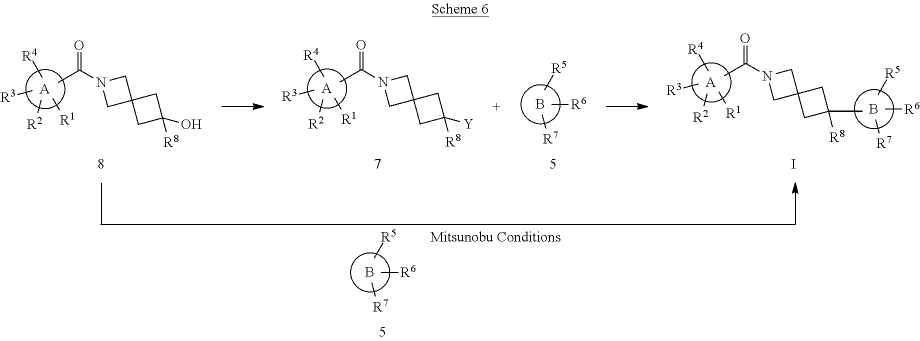

Alternatively, where X=CR⁸ and Ring B is a C-linked (hetero)aryl, compounds of formula I may be generated using a metal-catalyzed cross-coupling reaction from suitably functionalized intermediates 7 and 9, where one partner bears an organometallic (e.g. zincate, boronate) typically generated from a halide intermediate such as I or Br, and the other partner bears a halide such as Br or I. (Scheme 7) For example under Negishi conditions (palladium catalysis) a zincate 7 transiently generated from the iodide (7; Y¹═I) can be reacted with a (hetero)aryl halide 9 (Y²═Br, I). The iodide 7 can be generated from the related hydroxy building block 8 by reaction with 12 and PPh₃.

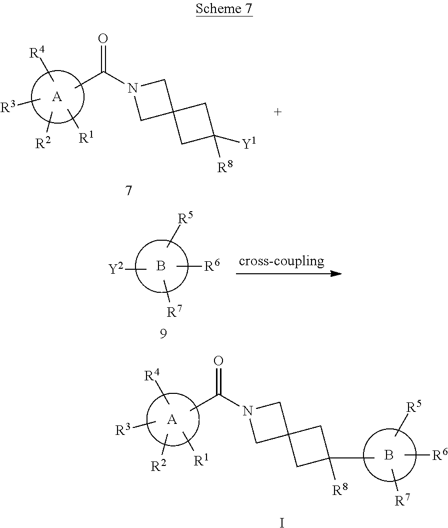

Alternatively, compounds of formula I with A=(substituted) pyridyl, (substituted) pyrimidinyl or (substituted) pyridazinyl with a (halo)alkoxy R group were prepared by reacting compounds of formula 10 (Y═F, Cl) with the corresponding alcohol and a suitable base (e.g. NaH or KOtBu) (Scheme 8). Alternatively, if Y═OH, compounds of formula I can be generated from the corresponding alcohol by a Mitsunobu reaction (for example, using cyanomethyl tributylphosphorane).

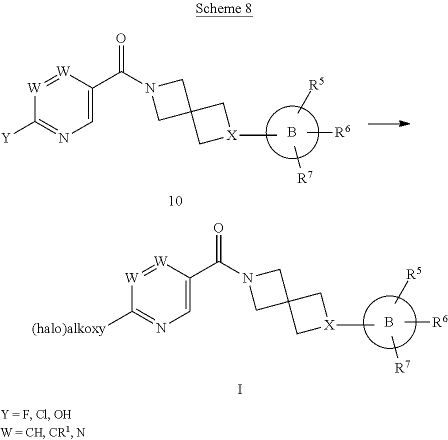

Alternatively, compounds of formula I with A=aliphatic (hetero)cycle and C=(hetero)aryl can be generated by coupling a boronic acid derivative 11 (Y=B(OR)₂) with an iodide 12 under nickel or palladium catalysis. Alternatively a bromide 11 (Y=Br) can be coupled directly with 12 in a photochemical reaction using Ir[dF(CF₃)ppy]₂(dtbbpy)PF₆, NiCl₂·DME, dtbbpy and (TMS)₃SiH. (Scheme 9)

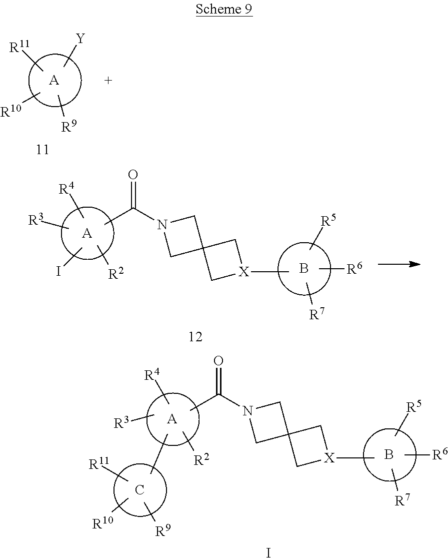

Alternatively, compounds of formula I with A=aliphatic (hetero)cycle, and L₁ is (or contains) oxygen can be prepared by reacting an alcohol of formula 13 (or other simple alcohol or haloalcohol group) with a suitably functionalized building block 14, where Y is a leaving group such as OMs or I, in an S_{N}2 reaction using a base such as NaH. (Scheme 10) The building block 14 can be generated from the corresponding alcohol (Y═OH, 16) if required.

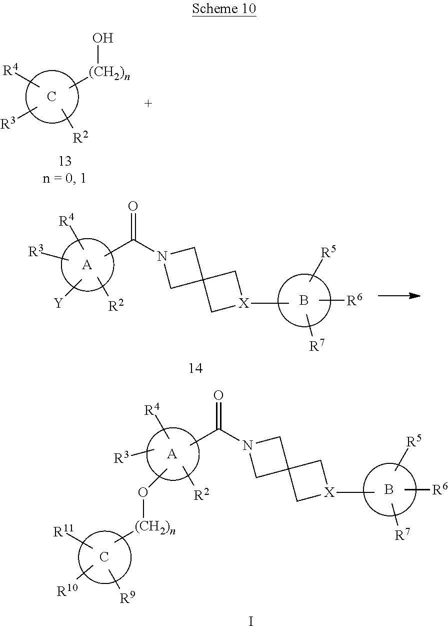

Alternatively, compounds of formula I with L₁ is (or contains) oxygen and C is a (substituted)aliphatic (hetero)cycle or a (hetero)aryl (where Y is in a position suitable for S_{N}Ar displacement), can be prepared by reacting 15 (Y is a leaving group such as OMs, Cl), with an alcohol 16 in the presence of a base such as NaH. (Scheme 11)

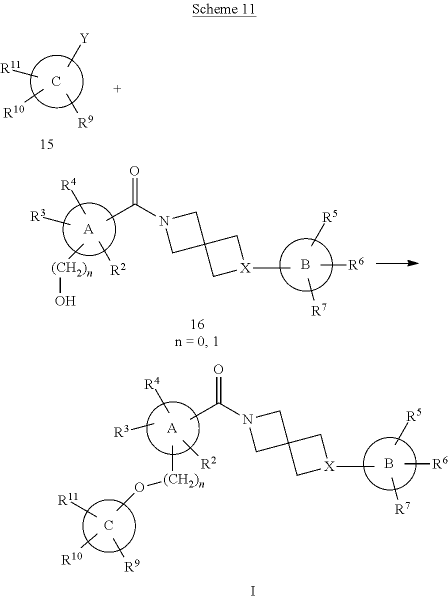

Building blocks of formula 18 with A=aliphatic (hetero)cycle, and L₁ is (or contains) oxygen can be prepared by reacting an alcohol of formula 13 (or other simple alcohol or haloalkyl-hydroxy group) with a suitably functionalized building block 17, where X¹ is a leaving group such as OMs or I and PG is a protecting group such as Boc, in an S_{N}2 reaction using a base such as NaH. The protecting group can then be removed under standard conditions (e.g. TFA for PG=Boc). (Scheme 12) The building block 17 can be generated from the corresponding alcohol (X¹=OH) if required. Alternatively, where the ring C is phenol-type (hetero)aryl (n=0), the building block 18 can be generated from an alcohol 17 (X¹═OH) using Mitsunobu conditions (e.g. PS—PPh₃, DIAD in THF) followed by deprotection.

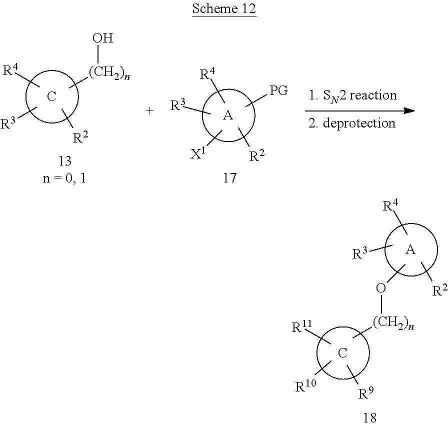

Alternatively, building blocks of formula 18 with L₁ is (or contains) oxygen and C is an aliphatic (hetero)cycle, a small (halo)alkyl fragament or a (hetero)aryl (where X¹ is in a position suitable for S_{N}Ar displacement), can be prepared by reacting 15 (X¹ is a leaving group such as OMs, Cl), with a suitably protected alcohol building block 19 in the presence of a base such as NaH or KOtBu, followed by deprotection under standard conditions (e.g. with TFA when PG=Boc). (Scheme 13) Alternatively, building blocks of formula 18 with L₁=oxygen and C is (hetero)aryl can be generated by a palladium-catalyzed cross coupling of the alcohol 19 with the (hetero)aryl halide 15 (X¹=typically Br, I), followed by deprotection.

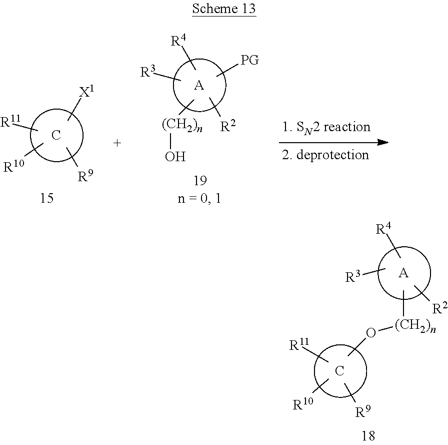

Building blocks of formula 21 with A=(substituted) pyridyl, (substituted) pyrimidinyl or (substituted) pyridazinyl with a (halo)alkoxy R group can be prepared by reacting a suitably protected (e.g. as methyl ester) heteroaryl acid of formula 20 (X¹=F, Cl) with the corresponding alcohol and a suitable base such as NaH or KOtBu as base in a solvent such as DMA, NMP or DMF at elevated temperature if required. If additional substitution R₂ is required on the heteroaryl, this can typically be installed via a commercial halide (R₂=Br, I), followed by a metal-catalyzed cross coupling (e.g. Suzuki, Negishi, Ir-catalyzed photochemical reaction). The protecting group (if used) can be removed under standard conditions (e.g. alkaline hydrolysis for the methyl ester). (Scheme 14) Alternatively, these building blocks can be generated by synthesizing a suitable bromo or iodo-heteroarene using standard techniques, followed by installation of the acid (or ester) functionality via a Pd-catalyzed carbonylation reaction.

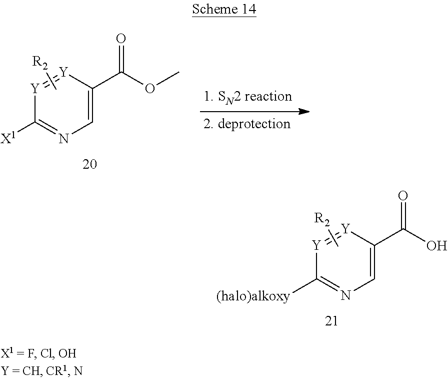

Where X=CR⁸ and ring B is a C-linked (hetero)aryl, amine 3 can be generated by reacting a carbanion (or carbanion equivalent) with a suitably protected building block, such as tert-butyl 6-oxo-2-azaspiro[3.3]heptane-2-carboxylate, followed by deprotection of the protecting group using standard conditions (e.g. using TFA where PG=Boc). The anion can be generated by direct metallation of a heteroaryl 22 (Y=H, e.g. with BuLi), or by metal-halogen exchange (Y=Br, I, e.g. with BuLi). (Scheme 15)

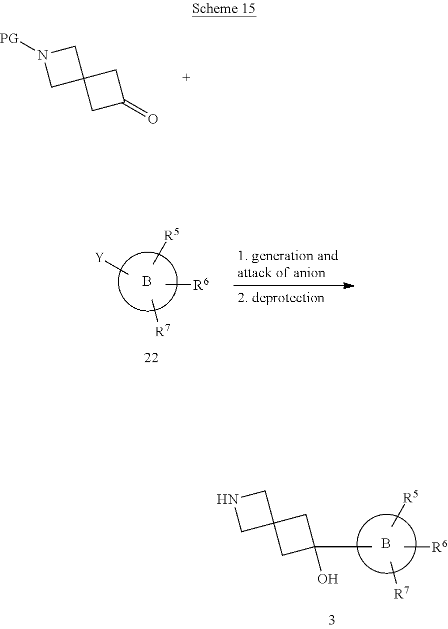

Building blocks of formula 24 with A=aliphatic (hetero)cycle and C=(hetero)aryl can be generated by coupling a suitably protected boronic acid derivative 11 (X¹=B(OR)₂) with an iodide 23 under nickel or palladium catalysis. Alternatively a bromide 11 (X¹=Br) can be coupled directly with 23 in a photochemical reaction using Ir[dF(CF₃)ppy]₂(dtbbpy)PF₆, NiCl₂·DME, dtbbpy and (TMS)₃SiH. (Scheme 16)

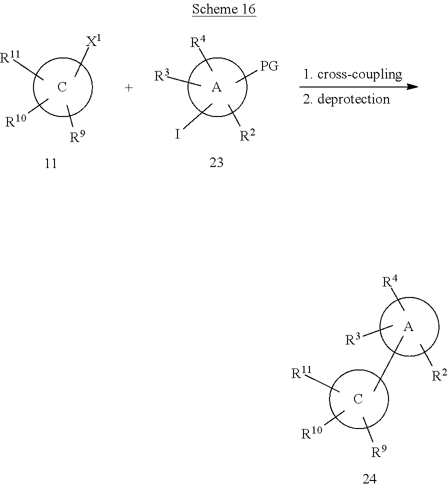

Building blocks of formula 27 where C=(hetero)aryl can be generated by Suzuki reaction (e.g. (Pd(dppf)Cl₂, K₂CO₃, dioxane/H₂O) of a (hetero)aryl halide (X¹═Br, I, Cl) followed by hydrogenation (e.g. Pd/C, H₂). The required boronate intermediate 25 can be generated by reacting a ketone with 4,4,5,5-tetramethyl-2-[(tetramethyl-1,3,2-dioxaborolan-2-yl)methyl]-1,3,2-dioxaborolane (LiTMP, THF, −78° C.). (Scheme 17). Alternatively the alkene 26 can be generated via Wittig reaction using the ketone and a suitable triphenylphosphonium bromide of Ring C (generated from the benzyl bromide, or heteroaryl equivalent). Alternatively, the building block 27 can be generated by generating a tosylhydrazone intermediate from an aldehyde on Ring A (e.g. by condensation with 4-methylbenzenesulfonhydrazide), followed by reaction with the relevant (hetero)aryl boronic acid (e.g. K₂CO₃, Barluenga conditions), before deprotection. Building blocks where L₁=CR¹²R¹³ and R¹²=carbamoyl, R¹³=hydrogen, can be synthetized via a similar strategy involving hydrogenation of an alkene intermediate, generated via condensation of a (hetero)aryl acetonitrile with a ketone (52), followed by hydrolysis of the nitrile and amide formation, using standard techniques.

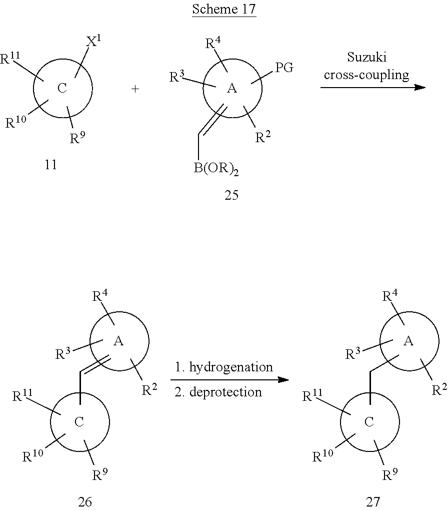

Building blocks of formula 30 with L₁ is oxygen and C is a phenol or (hetero)aryl hydroxy (28) can be prepared by reacting the nucleophilic (hetero)aryl hydroxy anion (generated using a base e.g. NaH) with a suitably protected building block 29 bearing a leaving group X¹ (e.g. OMs, which can be generated from the hydroxy derivative using MsCl, Et₃N) in a position suitable for S_{N}2 substitution. This can be followed by deprotection under standard conditions (e.g. with TFA when PG=Boc). (Scheme 18) Alternatively, the building blocks 30 can be generated via Mitsunobu reaction (using e.g. DIAD, PPh₃ or 2-(tributyl-15-phosphaneylidene)acetonitrile) of the phenol with an alcohol 29 (X¹═OH), followed by deprotection.

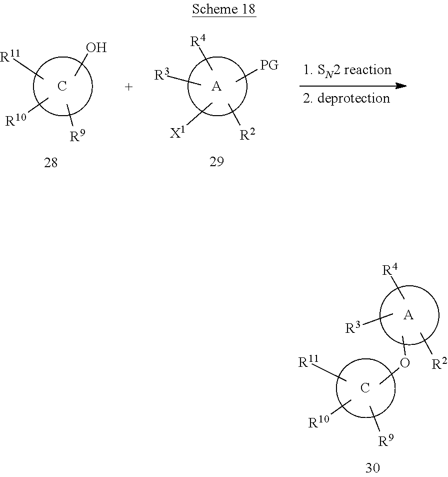

Alternatively, building blocks of formula 32 with A=aliphatic (hetero)cycle and C=(hetero)aryl can be generated by coupling a boronic acid derivative 11 (X¹═B(OR)₂) with a suitably protected halide (Y═I or Br) 31 under nickel or palladium catalysis. Alternatively a bromide 11 (X¹═Br) can be coupled directly with 31 (Y═I or Br) in a photochemical reaction using Ir[dF(CF₃)ppy]₂(dtbbpy)PF₆, NiCl₂·DME, dtbbpy and (TMS)₃SiH. The coupling is followed by a suitable deprotection step (e.g. TsOH, when PG=Boc). (Scheme 19)

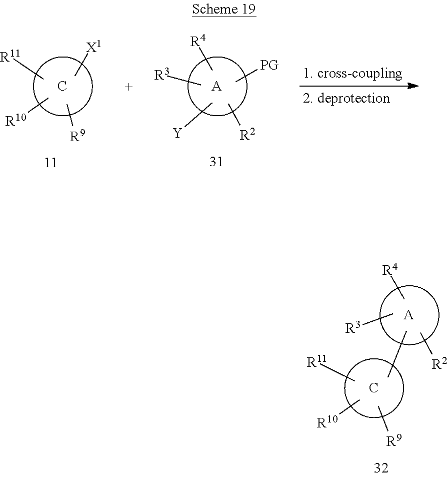

Alternatively, building blocks of formula 35 with A=aliphatic (hetero)cycle, L₁=—OCH₂— and C=(hetero)aryl can be generated by reacting a (hetero)aryl bearing a leaving group (e.g. X¹═Br) in a benzylic position (33) with an alcohol 34 in the presence of a base (e.g. NaH). The coupling is followed by a suitable deprotection step (e.g. TsOH or TFA, when PG=Boc). (Scheme 20) This route is also appropriate where C=small aliphatic (hetero)cycle (e.g. cyclopropyl), or where Ring C is replaced by a small alkyl or haloalkyl fragment.

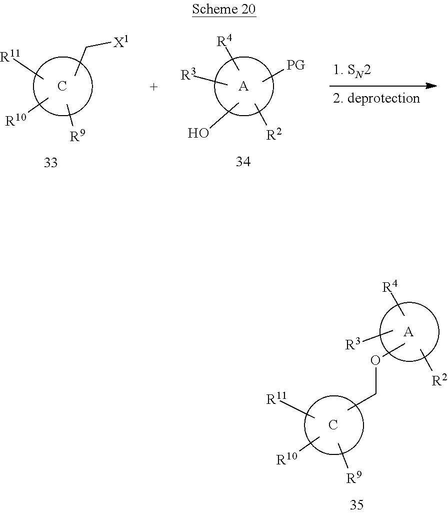

Alternatively, building blocks of formula 37 with L₁=C—C triple bond can be generated from a (hetero)aryl halide 11 (X¹═Br or I) and a suitably protected alkyne 36 via a Sonogashira coupling (e.g. Pd(PPh₃)₂C₁₂, CuI, TEA), followed by a suitable deprotection step (e.g. TsOH or TFA, when PG=Boc). (Scheme 21)

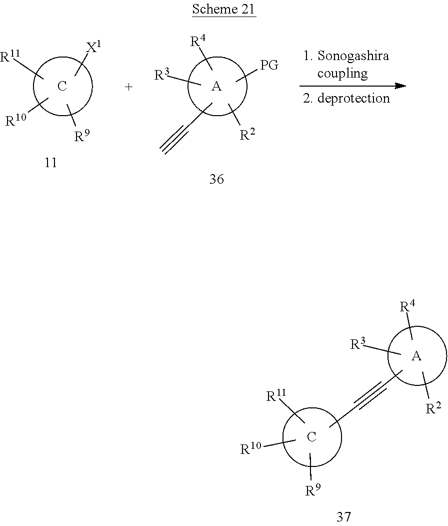

Building blocks of formula 38 with B=C-linked (hetero)aryl and X=CR⁸ can be generated by coupling a suitably protected boronic acid derivative 39 (Y═B(OR)₂) with an iodide or bromide (Z═I or Br) 40 under nickel or palladium catalysis. Alternatively a bromide 39 (Y═Br) can be coupled directly with 40 (Z═I or Br) in a photochemical reaction using Ir[dF(CF₃)ppy]₂(dtbbpy)PF₆, NiCl₂·DME, dtbbpy and (TMS)₃SiH. (Scheme 22) Alternatively the cross-coupling can be carried out under Negishi conditions with a zincate transiently generated from 39 (Y═I) and a (hetero)aryl halide 40 (Z═I, Br).

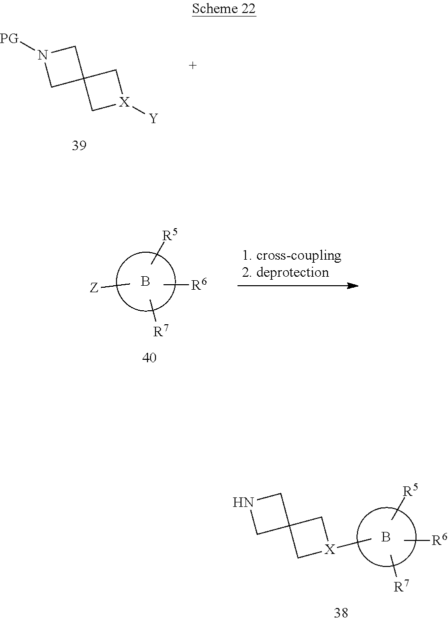

Building blocks of formula 41 with X═N and B=(hetero)aryl can be prepared using a metal-catalysed cross-coupling reaction (e.g. Buchwald reaction, Pd-catalysis) between suitably protected 42 and a (hetero)arylhalide (Y═Br, I, Cl), followed by deprotection under standard conditions (e.g. with TsOH or TFA when PG=Boc). (Scheme 23)

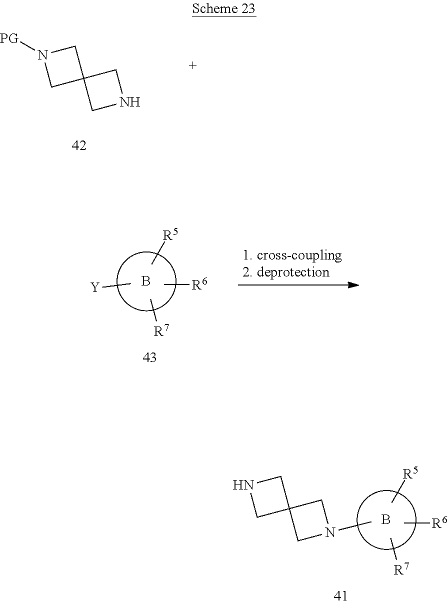

Alternatively, building blocks of formula 44 with L₁=—SO₂NH— can be prepared from a sulfonyl chloride 45 and suitably protected (spiro)cyclic amine (46) in the presence of a base such as DIPEA, followed by deprotection under standard conditions (e.g. with TsOH when PG=Boc). (Scheme 24) This sequence is also suitable where there is a small alkyl group in place of the C-ring.

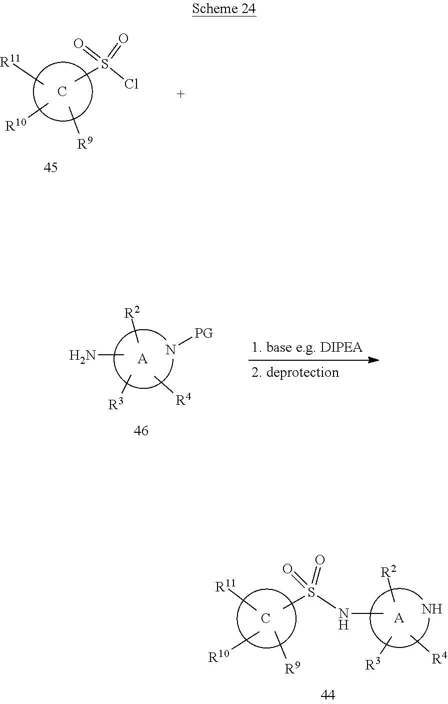

Alternatively, building blocks of formula 47 with L₁=NH and C is (hetero)aryl (where X¹ is in a position suitable for S_{N}Ar displacement) can be prepared by reacting 48 (X¹ is a leaving group such as C₁, Br, often adjacent to aromatic N for S_{N}Ar reaction), with a suitably protected amine building block 49 in the presence of a base such as DIPEA, followed by deprotection under standard conditions (e.g. with TsOH when PG=Boc). (Scheme 25)

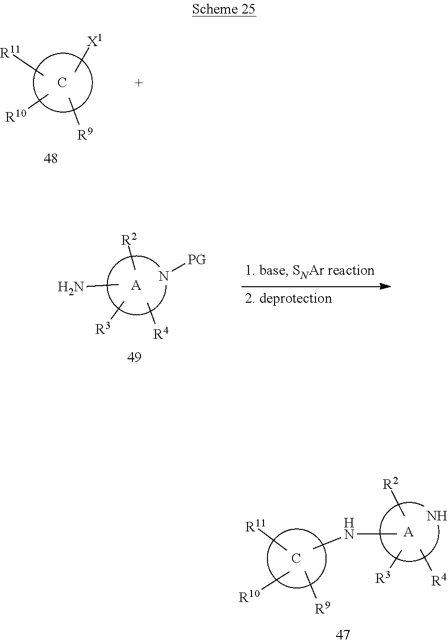

Alternatively, building blocks of formula 50 with L₁=NH₂ or CH₂NH may be installed by a reductive amination reaction of amine 51 with a suitably protected ketone building block 56 in the presence of a reducing agent such as sodium triacetoxyborohydride or sodium cyanoborohydride, followed by deprotection under standard conditions (e.g. with TsOH when PG=Boc). (Scheme 26)

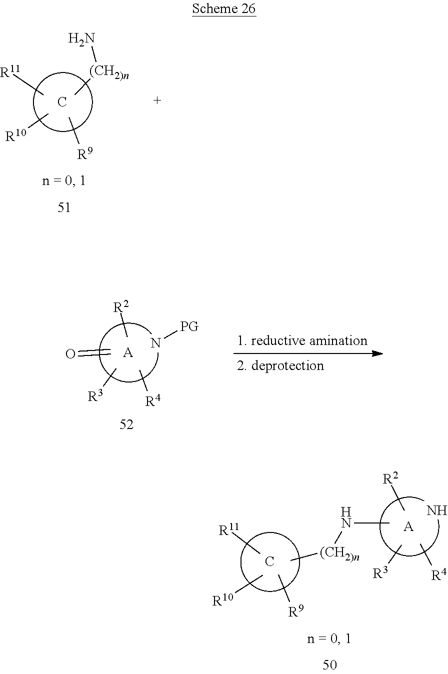

Alternatively, building blocks of formula 53 with L₁=CH₂ and A is N-linked can be prepared by a reductive amination reaction of aldehyde 54 with suitably protected heterocycle A (55) in the presence of a reducing agent such as sodium triacetoxyborohydride or sodium cyanoborohydride, followed by deprotection under standard conditions (e.g. with TsOH when PG=Boc). (Scheme 27) The same sequence can also be carried out to generate building blocks of formula 56 where with L₁=CH₂NH from aldehyde 54 and amine 57. (Scheme 28)

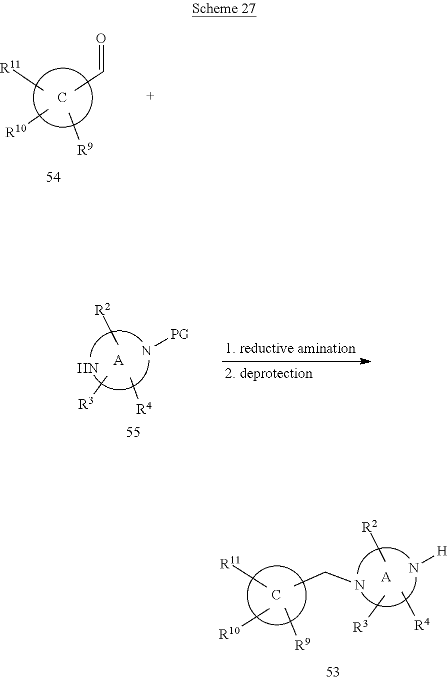

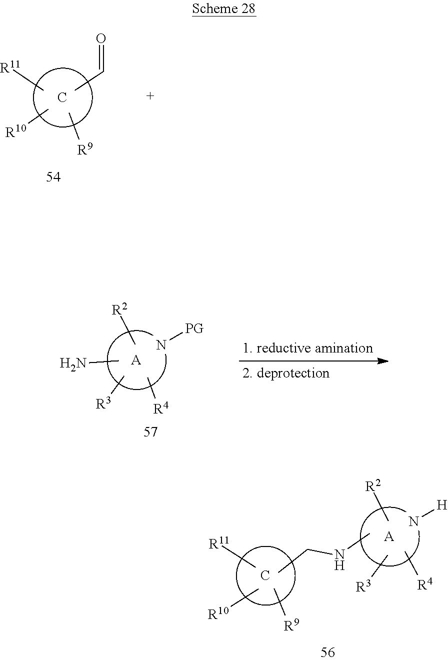

Alternatively, building blocks of formula 58 with L₁=SO₂ and A is N-linked can be prepared from a sulfonyl chloride 45 and suitably protected heterocycle A (55) in the presence of a base such as DIPEA, followed by deprotection under standard conditions (e.g. with TsOH when PG=Boc). (Scheme 29) This sequence is also appropriate where A is N-linked and R₁ is C₁-C₆-alkyl-SO₂—.

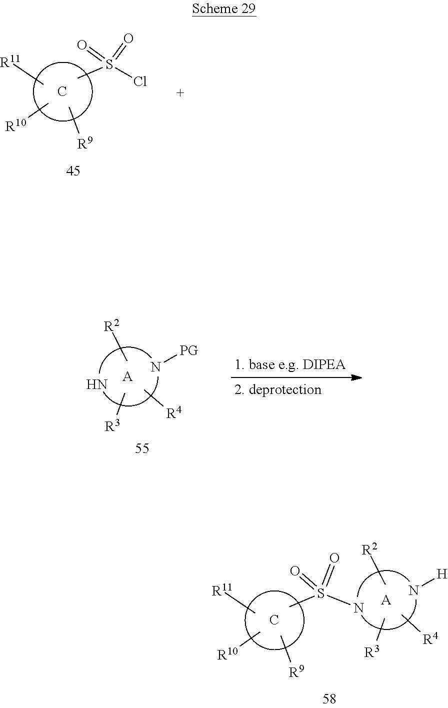

Alternatively, building blocks of formula 59 with L₁=CH₂ and A is N-linked can be prepared by a reductive amination reaction of (hetero)aryl methylhalide (X¹═Br, I) 60 with suitably protected heterocycle A (55) in the presence of a base such as K₂CO₃ in a solvent such as ACN, followed by deprotection under standard conditions (e.g. with TsOH when PG=Boc). (Scheme 30)

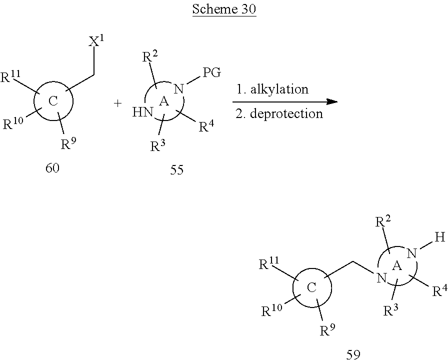

Alternatively, building blocks of formula 59 with L₁=CH₂ and A is N-linked can be prepared by a reductive amination reaction of acid chloride 61 with suitably protected heterocycle A (55) in the presense of a base (e.g. DIPEA) to form an amide, follow by reduction of the amide (e.g. using borane tetrahydrofuran complex), and deprotection under standard conditions (e.g. with TsOH when PG=Boc). (Scheme 31)

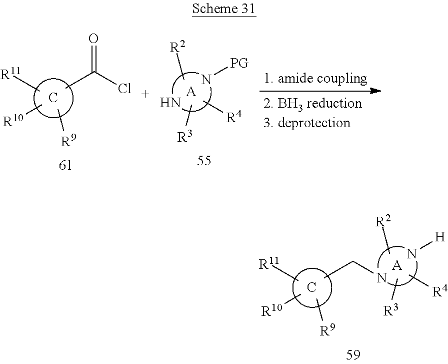

Alternatively, building blocks of formula 62 with L₁=C(O)NH could be prepared by reacting a suitably protected amine 57 with a carboxylic acid 63 to produce amide 45 using standard amide coupling techniques (e.g. HATU, DIPEA), followed by deprotection under standard conditions (e.g. with TsOH or TFA when PG=Boc). (Scheme 32)

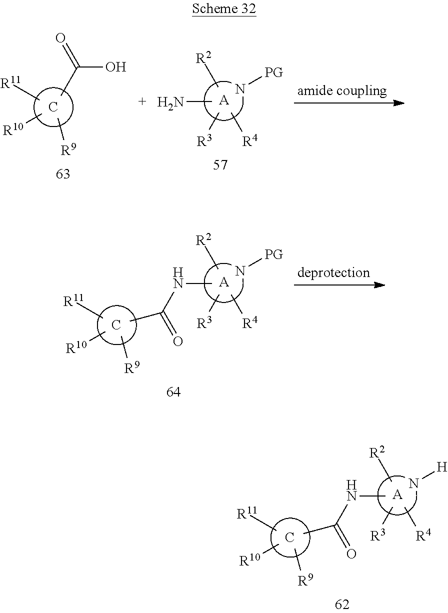

Alternatively, sulfonylurea building blocks of formula 65 with L¹=—NHSO₂— or —CH₂NHSO₂—, can be prepared by activating 2-methyl-1-(2-methylimidazol-1-yl)sulfonyl-imidazole by methylation with methyl trifluoromethanesulfonate, followed by reaction with suitably protected amine 55; a further sequence of activation by methylation with methyl trifluoromethanesulfonate followed by reaction with amine 51; and finally deprotection under standard conditions (e.g. with TsOH or TFA when PG=Boc). (Scheme 33) Alternatively sulfonylurea building blocks 65 can be generated from sulfuryl chloride followed by sequential additions of 55 and 51 in the presence of a base such as Et₃N or DIPEA, and finally deprotection under standard conditions.

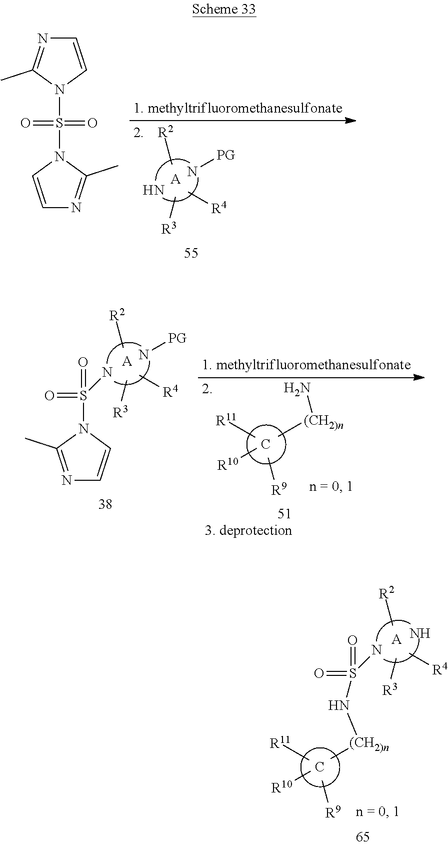

Building blocks with L₁=bond and C═C-linked (hetero)aryl and R⁹=amine (L₂=—NH— or —CH₂NH—and D is an aliphatic heterocycle such as cyclopropyl (66) or C═N-linked heterocycle (67)) can be generated by metal-catalyzed amination of intermediate 70 (X¹═Br, Cl adjacent to aromatic N) with the relevant amine building block 71 or 72 (typically Pd-catalysis, Buchwald reaction). The required intermediate 70 could be generated by a cross-coupling such as Negishi reaction between a zincate transiently generated from suitably protected building block 69 (Y═I) and a suitable dihalogenated (hetero)aryl building block 68 (X² needs to be more reactive to the cross coupling conditions than X¹; typically X²═I or Br, and X¹═Br). (Scheme 34) Alternatively the intermediate 70 can be generated by reacting a p-tolylsulfonylhydrazono derivative of 69 (Y═N—NH-Ts) with a boronic acid 68 (X²═B(OH)₂) in the presence of a base such as K₂CO₃.

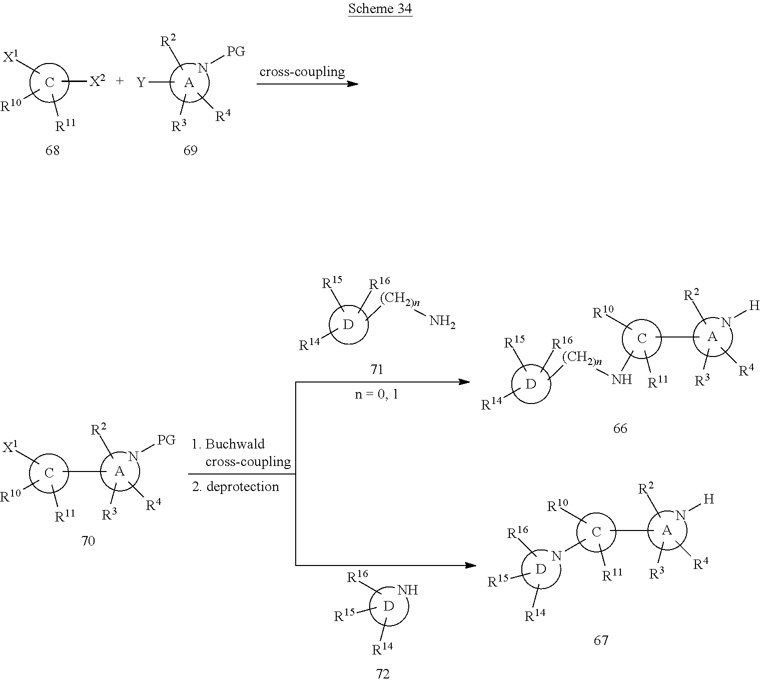

Alternatively, the amine 71 or 72 can be reacted with (hetero)aryl building block 68 (X¹═F) in the presence of a base (such as DIEA or K₂CO₃) in an S_{N}Ar reaction to generate intermediate 73 or 74, before carrying out the cross coupling reaction with the A ring (typically using photochemical reaction of 69 (Y═Br) with halide 73 or 74 using Ir[dF(CF₃)ppy]₂(dtbbpy)PF₆, NiCl₂·DME, dtbbpy and (TMS)₃SiH), followed by deprotection. (Scheme 35) Alternatively this S_{N}Ar approach to produce the R⁹ amine could be carried out on intermediates such as 70 (where X¹═F). (Scheme 34) Occasionally copper-catalyzed S_{N}Ar or Ullmann type reactions were used for the reaction of 68 and 72 to give 74.

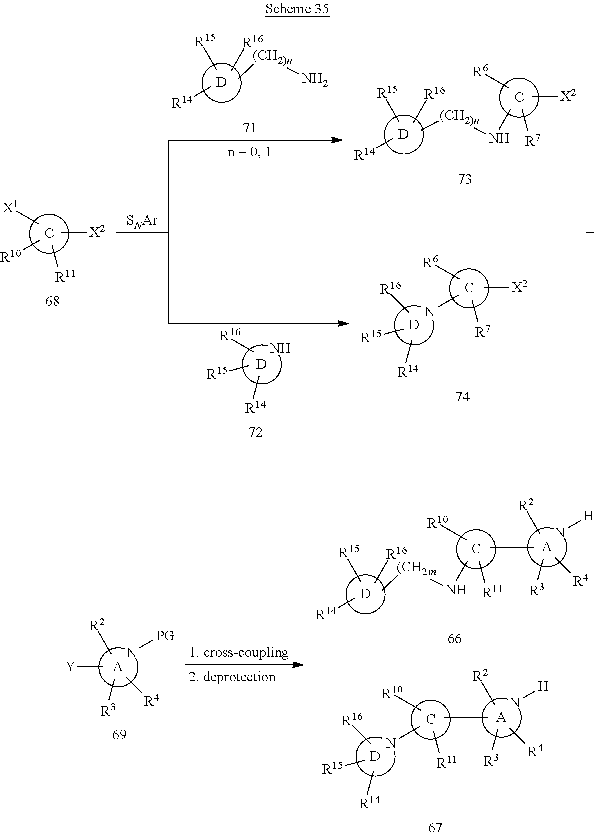

Building blocks with L₁=bond and C═C-linked (hetero)aryl and D=N-linked heteroaryl (57) can be generated by Chan-Lam coupling (in presence of Cu(OAc)₂) of a boronic acid 68 (X¹═B(OH)₂, X²═Br) with the heteroaryl to give intermediate 77, which could then be reacted with suitably protected (spiro)cyclic amine 69 (Y═Br) in a photochemical cross-coupling using Ir[dF(CF₃)ppy]₂(dtbbpy)PF₆, NiCl₂·DME, dtbbpy and (TMS)₃SiH), followed by deprotection. (Scheme 36) Alternatively, for C-linked heterocycles, the heterocycle C can be constructed via standard heterocyclic synthesis techniques prior to the photochemical cross coupling reaction and deprotection.

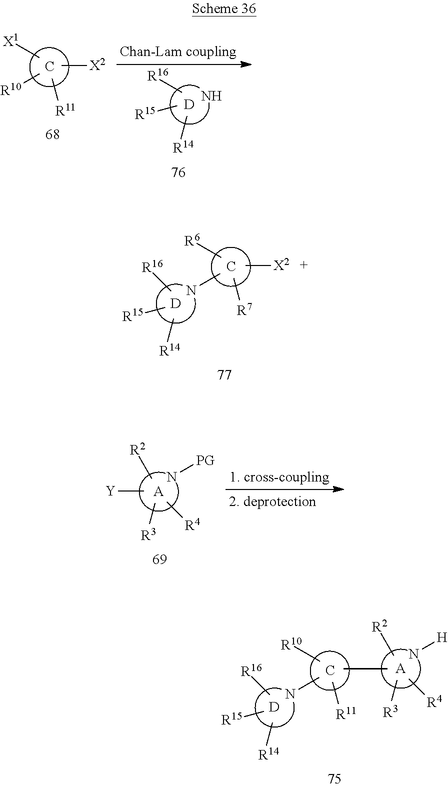

Alternatively, building blocks of formula 78 with L₁ and L₂=bond and C and D are (hetero)aryl can be prepared by reacting a suitably protected (spiro)cyclic amine (Y═I or Br) in two sequential cross coupling reactions, followed by deprotection. Most typically this involves a Ni-catalyzed cross-coupling between 69 (Y═I) and a building block 79 bearing both a bromide and boronic acid functionality to give bromo (hetero)aryl intermediate 80. Intermediate 80 could be coupled with a boronic acid derivative 81 under Suzuki conditions followed by deprotection to give 78. (Scheme 37)

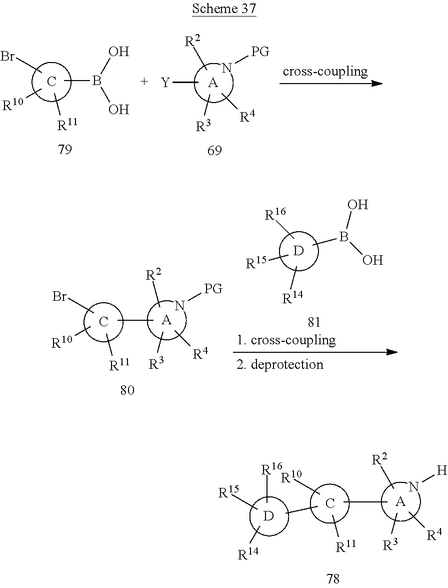

Building blocks of formula 82 where L₁=bond and L₂=—NHCH₂— can be generated by reductive amination reaction of suitably protected aldehyde 83 with an amine 84 in the presence of a reducing agent such as sodium triacetoxyborohydride or sodium cyanoborohydride, followed by deprotection under standard conditions (e.g. with TsOH when PG=Boc). (Scheme 38) If required, the aldehyde 83 can be generated from the acid via a reduction (e.g. using borane) and oxidation (e.g. using an oxidant such as DMP) sequence.

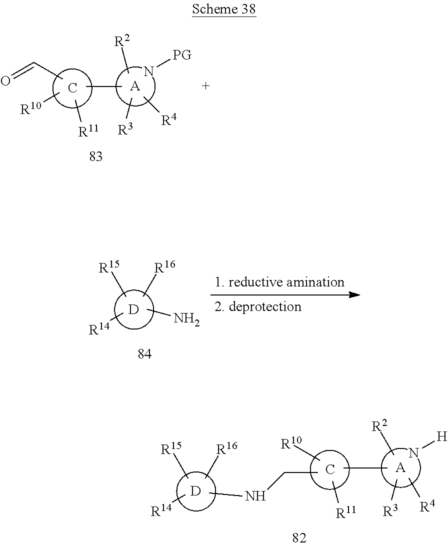

Building blocks of formula 85 where L₁=bond and L₂=—NHCH₂— can be generated by Curtius rearrangement of suitably protected acid building block 86 (e.g. using diphenylphosphonic azide, benzyl alcohol) and a deprotection of the generated carbamate (e.g. with Pd/C, H₂) to give amine 87, followed by further reductive amination of amine 87 with a suitable aldehyde 88, and subsequent deprotection. (Scheme 39) Alternatively the amine 87 can be used in a heterocylic synthesis reaction (e.g. condensation reaction with a suitable 1,3-dione O-(4-nitrobenzoyl)hydroxylamine to generate a pyrazole), to generate an N-linked heterocylic D ring.

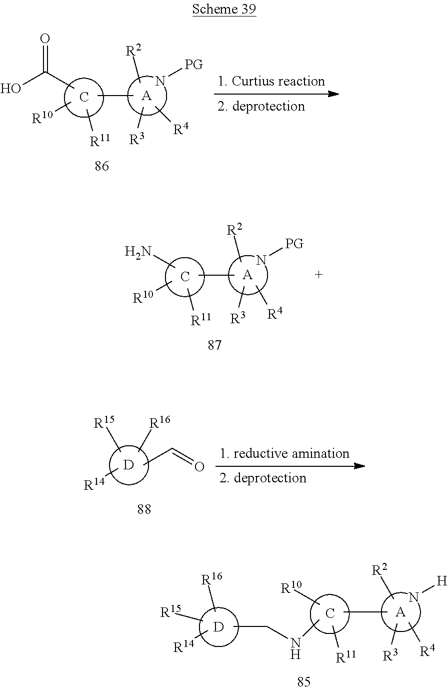

Building blocks of formula 89 where L₁=bond and L₂=bond and C=heteroaryl can be generated using standard heteroaryl synthesis techniques starting from a building block 90 bearing a cyano or carboxylic acid group (X¹═CN, COOH). (Scheme 40) A similar sequence can also be used to generate a heterocyclic E ring from a suitable R¹⁴ group on the D ring, for building blocks where L₃=bond and E=heteroaryl.

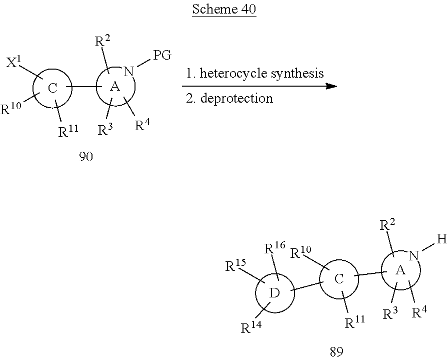

Alternatively, building blocks of formula 91 with L₁=—NHCH₂— may be installed by a reductive amination reaction of amine 92 with a suitably protected aldehyde building block 93 in the presence of a reducing agent such as sodium triacetoxyborohydride or sodium cyanoborohydride, followed by deprotection under standard conditions (e.g. with TsOH when PG=Boc). (Scheme 41)

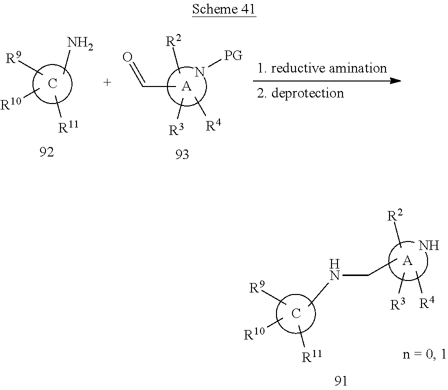

Alternatively, building blocks of formula 94 with L₁ and L₂=bond and B is (hetero)aryl and C is a N-linked cyclic amine, can be prepared by reacting a suitably protected (spiro)cyclic amine (Y═I or Br) in two sequential cross coupling reactions, followed by deprotection. Most typically this involves a Ni-catalyzed cross-coupling between 69 (Y═I) and a building block 79 bearing both a bromide and boronic acid functionality to give bromo (hetero)aryl intermediate 95. Intermediate 95 could be coupled with an amine 96 (e.g. under Buchwald conditions, Pd-catalysis) followed by deprotection to give 94. (Scheme 42)

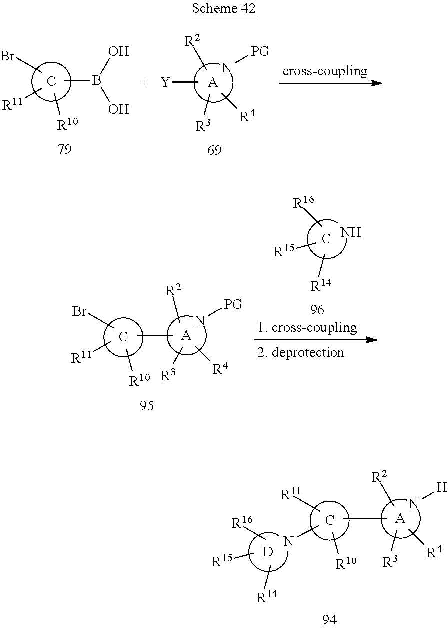

Building blocks of formula 97, with L₁ and L₂=bond, C=(hetero)aryl and D=C₃-C₁₀-cycloalkyl, 3- to 14-membered heterocyclyl, can be prepared by reacting a suitably protected (spiro)cyclic amine 69 (Y═I or Br) in two sequential cross coupling reactions, followed by deprotection. Most typically this involves a Negishi cross-coupling with a zincate transiently generated from 69 (Y═I) and a building block 98 bearing two bromide functionalities, to give bromo (hetero)aryl intermediate 99. Cross-coupling between intermediate 99 and bromide 100 in a photochemical reaction using Ir[dF(CF₃)ppy]₂(dtbbpy)PF₆, NiCl₂·DME, dtbbpy and (TMS)₃SiH followed by deprotection generates building blocks of general formula 97. (Scheme 43)

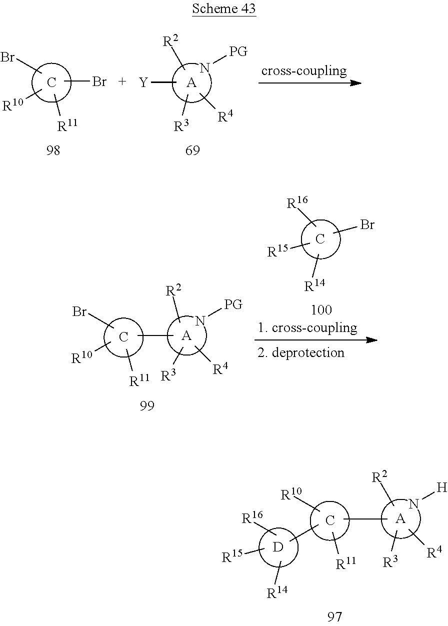

Building blocks of formula 101 where L₂=CH₂ and D=heteroaryl may be generated via a Amdt-Eistert type homologation sequence of carboxylic acid 90 to generate homologated acid 102, which could then be further derivatized, for example using standard heterocyclic synthesis techniques, followed by deprotection. (Scheme 44)

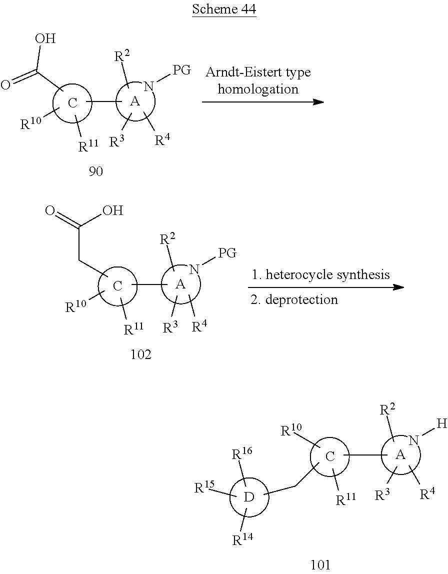

Building blocks of formula 1 where L₁=—CH₂CH₂— can be generated via a Wittig-type coupling between the A and C rings to generate an alkene, followed by reduction.

Building blocks of formula 1 where C is a cyclic amine, D=(hetero)aryl, L₁=bond, and L₂=—CH₂— N-linked to D-ring can be generated via reductive amination in a similar process as to that depicted above.

In some cases, compounds of formula I could also be generated by combining the steps already described in new combinations e.g. carrying out the coupling in Scheme 1 prior to elaboration of the individual building blocks using the same sequences as described above.

In some cases, compounds of formula I could be further functionalized to give other compounds of formula I. For example, a compound of formula I bearing a (hetero)aryl bromide or iodide can be further functionalized with other groups e.g. small amine, small alkyl using metal catalyzed cross-coupling conditions such as Buchwald or Suzuki reactions. Building blocks 1 can also be subjected to further functionalization reactions (e.g., formation of an amide under standard conditions, alkylation of an alcohol (e.g. using NaH and an alkylating agent in DMF), conversion of boron-containing groups to hydroxyl using alkaline peroxide conditions, oxidation of thioethers to sulfones, or installation of small alkyl groups in place of Br or I groups using metal catalyzed cross-coupling conditions such as Buchwald or Suzuki reactions) before or after deprotection of the nucleophilic amine, to yield other building blocks of formula 1.

In some cases, building blocks could be generated from commercially available fragments using standard functional group interconversion techniques (e.g. conversion of halides to other groups e.g. small amine, small alkyl using metal catalyzed cross-coupling conditions such as Buchwald or Suzuki reactions, conversion of boron-containing groups to hydroxyl using alkaline peroxide conditions, cycloaddition of azidotrimethylsilane with a nitrile to generate a tetrazole, Sandmeyer reaction of an aniline to a bromide, oxidation of thioethers to sulfones, alkylation of hydroxyl or amine groups via S_{N}2 reaction or reductive amination, acylation using an activated carbonyl derivative, or installation of —SO₂Me or —SO₂CF₃ groups from a iodo- or bromo-building block using literature techniques). Such techniques may also be used to elaborate commercially available fragments before, after, or intermediate within the synthetic sequences described above.

Alternatively, and especially where C=5-membered-ring heteroaryl, building blocks of can be prepared using standard heterocyclic synthesis techniques from suitably functionalized and protected A-ring precursors (see for example Heterocyclic Chemistry, Joule J. A. and Mills K., 5^{th} Edition, Wiley, 2010). As example: Where C=1,2,4-oxadiazole, building blocks can be generated from an A-ring bearing a carboxylic acid derivative via condensation/cyclization with an alkyl N-hydroxyacetamidine (which can be prepared from an alkylnitrile and hydroxylamine hydrochloride). Regioisomeric 1,2,4-oxadiazoles can also be generated by a similar process, using an A-ring bearing a nitrile group and a (halo)alkylcarboxylic acid. Where C=1,3,4-oxadiazole, the building block can be prepared from an A-ring bearing a carboxylic acid derivative via generation of a hydrazinecarbonyl derivative and condensation/cyclization with a (halo)alkylcarboxylic acid. Where C=1,2,3-triazole, the building block can be prepared from a A-ring bearing an acetylene derivative and a (halo)alkylazide, transiently generated from the amine via a cycloaddition reaction.

Alternatively, when C=heteroaryl and particularly where the A-ring=(hetero)aryl, and ^{L}=single bond, the building blocks could be generated by a metal-catalyzed cross coupling such as a Buchwald or Ullman-type reaction (for N-linked C-rings) or Suzuki reaction.

In one aspect, the present invention provides a process of manufacturing the compounds of formula (I) described herein, or pharmaceutically acceptable salts thereof, wherein the process is as described in any one of schemes 1 to 44.

In one aspect, the present invention provides a compound of formula (I) as described herein, or a pharmaceutically acceptable salt thereof, when manufactured according to any one of the processes described herein.

MAGL Inhibitory Activity

Compounds of the present invention are MAGL inhibitors. Thus, in one aspect, the present invention provides the use of compounds of formula (I) as described herein for inhibiting MAGL in a mammal.

In a further aspect, the present invention provides compounds of formula (I) as described herein for use in a method of inhibiting MAGL in a mammal.

In a further aspect, the present invention provides the use of compounds of formula (I) as described herein for the preparation of a medicament for inhibiting MAGL in a mammal.

In a further aspect, the present invention provides a method for inhibiting MAGL in a mammal, which method comprises administering an effective amount of a compound of formula (I) as described herein to the mammal.

Compounds of formula (I) were profiled for MAGL inhibitory activity by determining the enzymatic activity by following the hydrolysis of the natural substrate 2-arachidonoylglycerol (2-AG) resulting in arachidonic acid, which can be followed by mass spectrometry. This assay is hereinafter abbreviated “2-AG assay”.

Compounds of formula (I) were profiled for MAGL inhibitory activity by determining the enzymatic activity by following the hydrolysis of the natural substrate 2-arachidonoylglycerol (2-AG) resulting in arachidonic acid, which can be followed by mass spectrometry. This assay is hereinafter abbreviated “2-AG assay”. The 2-AG assay was carried out in 384 well polypropylene assay plates. Compound dilutions were made in 100% DMSO in a polypropylene plate in 3-fold dilution steps to give a final concentration range in the assay from 12.5 μM to 0.8 pM. Compound dilutions were added to MAGL protein in assay buffer (50 mM TRIS, 1 mM EDTA, 0.01% (v/v) Tween-20, 2.5% (v/v) DMSO). After shaking, the plate was incubated for 15 min at RT. To start the reaction, 2-arachidonoylglycerol in assay buffer was added. The final concentrations in the assay was 50 μM for MAGL protein and 8 μM 2-arachidonoylglyerol. After shaking and 30 min incubation at RT, the reaction was quenched by the addition of two assay volumes of acetonitrile containing 4 μM of d8-arachidonic acid. The amount of arachidonic acid formed was traced by an online SPE system (Agilent Rapidfire) coupled to a triple quadrupole mass spectrometer. A C18 SPE cartridge (Agilent G9205A) was used in an acetonitrile/water liquid setup. The mass spectrometer was operated in negative electrospray mode following the mass transitions 303.1→259.1 for arachidonic acid and 311.1→267.0 for d8-arachidonic acid. The activity of the compounds was calculated based on the ratio of intensities [arachidonic acid/d8-arachidonic acid].

TABLE 1

| | IC50 MAGL |
|---|---|
| Ex. | [μM] |
| 1 | 0.0001 |
| 2 | 0.0257 |
| 3 | 0.0051 |
| 4 | 0.0114 |
| 5 | 0.0859 |
| 6 | 0.1912 |
| 7 | 0.2146 |
| 8 | 0.4854 |
| 9 | 0.6130 |
| 10 | 0.0331 |
| 11 | 1.2875 |
| 12 | 0.0367 |
| 13 | 0.1521 |
| 14 | 0.1966 |
| 15 | 2.6715 |
| 16 | 2.8150 |
| 17 | 3.3645 |
| 18 | 3.7308 |
| 19 | 4.1667 |
| 20 | 0.0820 |
| 21 | 3.3740 |
| 22 | 0.4511 |
| 23 | 0.2291 |
| 24 | 0.0077 |
| 25 | 0.0122 |
| 26 | 0.0198 |
| 27 | 0.0265 |
| 28 | 0.0500 |
| 29 | 0.0522 |
| 30 | 0.1083 |
| 31 | 0.3530 |
| 32 | 2.8225 |
| 33 | 0.0004 |
| 34 | 0.0057 |
| 35 | 0.0200 |
| 36 | 0.0004 |
| 37 | 0.0245 |
| 38 | 0.0006 |
| 39 | 0.0024 |
| 40 | 0.00003 |
| 41 | 0.00004 |
| 42 | 0.0001 |
| 43 | 0.0001 |
| 44 | 0.0002 |
| 45 | 0.0002 |
| 46 | 0.0002 |
| 47 | 0.0002 |
| 48 | 0.0002 |
| 49 | 0.0003 |
| 50 | 0.0003 |
| 51 | 0.0003 |
| 52 | 0.0004 |
| 53 | 0.0007 |
| 54 | 0.0012 |
| 55 | 0.0018 |
| 56 | 0.0025 |
| 57 | 0.0025 |
| 58 | 0.0026 |
| 59 | 0.0026 |
| 60 | 0.0053 |
| 61 | 0.0054 |
| 62 | 0.0072 |
| 63 | 0.0198 |
| 64 | 0.0217 |
| 65 | 0.0832 |
| 66 | 0.1595 |
| 67 | 3.1682 |
| 68 | 0.0009 |
| 69 | 0.0020 |
| 70 | 0.0021 |
| 71 | 0.0071 |
| 72 | 0.0039 |
| 73 | 0.0082 |
| 74 | 0.1598 |
| 75 | 0.0027 |
| 76 | 0.0022 |
| 77 | 0.0092 |
| 78 | 0.0122 |
| 79 | 0.0013 |
| 80 | 0.0022 |
| 81 | 0.0018 |
| 82 | 0.0037 |
| 83 | 0.0052 |
| 84 | 0.0006 |
| 85 | 0.0004 |
| 86 | 0.0004 |
| 87 | 0.0065 |
| 88 | 0.0011 |
| 89 | 0.0025 |
| 90 | 0.0040 |
| 91 | 0.0063 |
| 92 | 0.0117 |
| 93 | 0.0010 |
| 94 | 0.0002 |
| 95 | 0.0012 |
| 96 | 0.0027 |
| 97 | 0.0034 |
| 98 | 0.0028 |
| 99 | 0.0095 |
| 100 | 0.0799 |
| 101 | 0.0421 |
| 102 | 0.0514 |
| 103 | 0.0099 |
| 104 | 0.0097 |
| 105 | 0.0039 |
| 106 | 0.0041 |
| 107 | 0.0047 |
| 108 | 0.0250 |
| 109 | 0.0635 |
| 110 | 0.0658 |
| 111 | 0.0043 |
| 112 | 0.0060 |
| 113 | 0.0048 |
| 114 | 0.0104 |
| 115 | 0.0024 |
| 116 | 0.0027 |
| 117 | 0.0035 |
| 118 | 0.0047 |
| 119 | 0.0074 |
| 120 | 0.0010 |
| 121 | 0.0054 |
| 122 | 0.0073 |
| 123 | 0.0419 |
| 124 | 0.0040 |
| 125 | 0.0046 |
| 126 | 0.0015 |
| 127 | 0.0144 |
| 128 | 0.0018 |
| 129 | 0.0722 |
| 130 | 0.0476 |
| 131 | 0.0059 |
| 132 | 0.0017 |
| 133 | 0.0013 |
| 134 | 0.0043 |
| 135 | 0.0031 |
| 136 | 0.0140 |
| 137 | 0.0045 |
| 138 | 0.0008 |
| 139 | 0.0115 |
| 140 | 0.0617 |
| 141 | 0.3324 |
| 142 | 0.0313 |
| 143 | 0.0532 |
| 144 | 0.6015 |
| 145 | 0.5413 |
| 146 | 0.4002 |
| 147 | 0.3397 |
| 148 | 1.4083 |
| 149 | 0.4407 |
| 150 | 0.1621 |
| 151 | 0.0671 |
| 152 | 0.3120 |
| 153 | 0.0149 |
| 154 | 0.0100 |
| 155 | 0.0208 |
| 156 | 0.0339 |
| 157 | 0.0437 |
| 158 | 0.0691 |
| 159 | 0.3146 |
| 160 | 1.0123 |
| 161 | 0.1844 |
| 162 | 0.4747 |
| 163 | 0.1916 |
| 164 | 0.0646 |
| 165 | 0.1023 |
| 166 | 0.0979 |
| 167 | 0.0855 |
| 168 | 1.4910 |
| 169 | 0.6589 |
| 170 | 0.2861 |
| 171 | 0.0964 |
| 172 | 0.1701 |
| 173 | 0.5465 |
| 174 | 0.1832 |
| 175 | 0.2210 |
| 176 | 1.4384 |
| 177 | 0.1411 |
| 178 | 0.1561 |
| 179 | 1.0286 |
| 180 | 4.6927 |
| 181 | 4.6145 |
| 182 | 2.5832 |
| 183 | 0.0001 |
| 184 | 0.0001 |
| 185 | 0.0001 |
| 186 | 0.0001 |
| 187 | 0.0002 |
| 188 | 0.0002 |
| 189 | 0.0002 |
| 190 | 0.0002 |
| 191 | 0.0002 |
| 192 | 0.0002 |
| 193 | 0.0003 |
| 194 | 0.0003 |
| 195 | 0.0004 |
| 196 | 0.0004 |
| 197 | 0.0004 |
| 198 | 0.0005 |
| 199 | 0.0005 |
| 200 | 0.0007 |
| 201 | 0.0007 |
| 202 | 0.0007 |
| 203 | 0.0008 |
| 204 | 0.0008 |
| 205 | 0.0009 |
| 206 | 0.0009 |
| 207 | 0.0010 |
| 208 | 0.0011 |
| 209 | 0.0012 |
| 210 | 0.0013 |
| 211 | 0.0014 |
| 212 | 0.0015 |
| 213 | 0.0016 |
| 214 | 0.0016 |
| 215 | 0.0017 |
| 216 | 0.0020 |
| 217 | 0.0020 |
| 218 | 0.0021 |
| 219 | 0.0022 |
| 220 | 0.0025 |
| 221 | 0.0025 |
| 222 | 0.0026 |
| 223 | 0.0030 |
| 224 | 0.0030 |
| 225 | 0.0033 |
| 226 | 0.0036 |
| 227 | 0.0038 |
| 228 | 0.0042 |
| 229 | 0.0045 |
| 230 | 0.0046 |
| 231 | 0.0046 |
| 232 | 0.0046 |
| 233 | 0.0053 |
| 234 | 0.0054 |
| 235 | 0.0060 |
| 236 | 0.0062 |
| 237 | 0.0064 |
| 238 | 0.0065 |
| 239 | 0.0066 |
| 240 | 0.0068 |
| 241 | 0.0087 |
| 242 | 0.0088 |
| 243 | 0.0088 |
| 244 | 0.0090 |
| 245 | 0.0099 |
| 246 | 0.0122 |
| 247 | 0.0122 |
| 248 | 0.0134 |
| 249 | 0.0144 |
| 250 | 0.0151 |
| 251 | 0.0155 |
| 252 | 0.0161 |
| 253 | 0.0162 |
| 254 | 0.0170 |
| 255 | 0.0172 |
| 256 | 0.0173 |
| 257 | 0.0180 |
| 258 | 0.0194 |
| 259 | 0.0218 |
| 260 | 0.0231 |
| 261 | 0.0242 |
| 262 | 0.0252 |
| 263 | 0.0272 |
| 264 | 0.0272 |
| 265 | 0.0275 |
| 266 | 0.0290 |
| 267 | 0.0307 |
| 268 | 0.0321 |
| 269 | 0.0322 |
| 270 | 0.0327 |
| 271 | 0.0338 |
| 272 | 0.0343 |
| 273 | 0.0344 |
| 274 | 0.0348 |
| 275 | 0.0353 |
| 276 | 0.0357 |
| 277 | 0.0372 |
| 278 | 0.0373 |
| 279 | 0.0380 |
| 280 | 0.0394 |
| 281 | 0.0413 |
| 282 | 0.0446 |
| 283 | 0.0483 |
| 284 | 0.0499 |
| 285 | 0.0543 |
| 286 | 0.0586 |
| 287 | 0.0594 |
| 288 | 0.0620 |
| 289 | 0.0621 |
| 290 | 0.0707 |
| 291 | 0.0762 |
| 292 | 0.0771 |
| 293 | 0.0780 |
| 294 | 0.0816 |
| 295 | 0.0890 |
| 296 | 0.0915 |
| 297 | 0.0944 |
| 298 | 0.0955 |
| 299 | 0.0957 |
| 300 | 0.0957 |
| 301 | 0.0973 |
| 302 | 0.1008 |
| 303 | 0.1009 |
| 304 | 0.1077 |
| 305 | 0.1173 |
| 306 | 0.1291 |
| 307 | 0.1355 |
| 308 | 0.1378 |
| 309 | 0.1484 |
| 310 | 0.1543 |
| 311 | 0.1758 |
| 312 | 0.2018 |
| 313 | 0.2062 |
| 314 | 0.2285 |
| 315 | 0.2404 |
| 316 | 0.2475 |
| 317 | 0.2736 |
| 318 | 0.2908 |
| 319 | 0.3008 |
| 320 | 0.3144 |
| 321 | 0.3308 |
| 322 | 0.3401 |
| 323 | 0.3650 |
| 324 | 0.4021 |
| 325 | 0.4819 |
| 326 | 0.5831 |
| 327 | 0.6110 |
| 328 | 0.6731 |
| 329 | 0.7431 |
| 330 | 0.7441 |
| 331 | 0.7965 |
| 332 | 0.9039 |
| 333 | 0.9490 |
| 334 | 0.9928 |
| 335 | 1.4037 |
| 336 | 1.4418 |
| 337 | 1.8291 |
| 338 | 2.1516 |
| 339 | 3.0054 |
| 340 | 3.1875 |
| 341 | 0.45445 |
| 342 | 0.33044 |
| 343 | 0.49126 |
| 344 | 0.00162 |
| 345 | 0.02888 |
| 346 | 0.01568 |
| 347 | 0.83273 |
| 348 | 0.06634 |
| 349 | 0.01192 |
| 350 | 0.01278 |
| 351 | 0.02470 |
| 352 | 0.00436 |
| 353 | 0.00240 |
| 354 | 0.01918 |
| 355 | 0.13351 |
| 356 | 1.08791 |
| 357 | 0.00201 |
| 358 | 0.03059 |
| 359 | 0.00100 |
| 360 | 0.00013 |
| 361 | 0.00021 |
| 362 | 0.00173 |
| 363 | 0.00135 |
| 364 | 0.00184 |
| 365 | 0.00183 |
| 366 | 0.01420 |
| 367 | 0.00238 |
| 368 | 0.00157 |
| 369 | 0.04926 |
| 370 | 0.00090 |
| 371 | 0.00923 |
| 372 | 0.00112 |
| 373 | 0.00324 |
| 374 | 0.05291 |
| 375 | 0.01269 |
| 376 | 0.00407 |
| 377 | 0.39151 |
| 378 | 0.10569 |
| 379 | 3.34654 |
| 380 | 0.00057 |
| 381 | 0.00044 |
| 382 | 0.05241 |
| 383 | 0.03978 |
| 384 | 0.00488 |
| 385 | 0.00125 |
| 386 | 0.14192 |
| 387 | 0.19558 |
| 388 | 0.00872 |
| 389 | 0.00868 |
| 390 | 0.03010 |
| 391 | 0.01414 |
| 392 | 0.19749 |
| 393 | 0.40074 |
| 394 | 0.04366 |
| 395 | 0.01465 |
| 396 | 0.00026 |
| 397 | 0.01436 |
| 398 | 0.03482 |
| 399 | 0.10179 |
| 400 | 0.00465 |
| 401 | 0.00945 |
| 402 | 0.00391 |
| 403 | 0.00125 |
| 404 | 0.00233 |
| 405 | 0.16911 |
| 406 | 0.00163 |
| 407 | 0.00025 |
| 408 | 0.00031 |
| 409 | 0.02746 |
| 410 | 0.02494 |
| 411 | 0.01634 |
| 412 | 0.00796 |
| 413 | 0.04784 |
| 414 | 2.96359 |
| 415 | 1.05961 |
| 416 | 0.00112 |
| 417 | 0.35853 |
| 418 | 0.00475 |
| 419 | 0.00248 |
| 420 | 0.00074 |
| 421 | 0.00076 |
| 422 | 0.00281 |
| 423 | 0.03848 |
| 424 | 0.00016 |
| 425 | 0.00555 |
| 426 | 0.00070 |
| 427 | 0.00053 |
| 428 | 0.05853 |
| 429 | 0.00066 |
| 430 | 0.01721 |
| 431 | 0.01032 |
| 432 | 0.00830 |
| 433 | 0.04406 |
| 434 | 0.00337 |
| 435 | 0.00769 |
| 436 | 0.00982 |
| 437 | 0.00637 |
| 438 | 0.01488 |
| 439 | 0.00374 |
| 440 | 0.00188 |
| 441 | 0.00197 |
| 442 | 0.00222 |
| 443 | 0.00431 |
| 444 | 0.00005 |
| 445 | 0.00030 |
| 446 | 0.00006 |
| 447 | 0.00002 |
| 448 | 0.00093 |
| 449 | 0.00137 |
| 450 | 0.07136 |
| 451 | 0.03173 |
| 452 | 0.00369 |
| 453 | 0.03937 |
| 454 | 0.00071 |
| 455 | 0.00125 |
| 456 | 0.00116 |
| 457 | 0.01097 |
| 458 | 0.01945 |
| 459 | 0.00030 |
| 460 | 0.00093 |
| 461 | 0.00179 |
| 462 | 0.00294 |
| 463 | 0.00242 |
| 464 | 0.00505 |
| 465 | 0.00317 |
| 466 | 0.00117 |
| 467 | 0.00236 |
| 468 | 0.00015 |
| 469 | 0.00048 |
| 470 | 0.00042 |
| 471 | 0.00069 |
| 472 | 0.04530 |
| 473 | 0.01140 |
| 474 | 0.02453 |
| 475 | 0.00044 |
| 476 | 0.00056 |
| 477 | 0.00097 |
| 478 | 0.00074 |
| 479 | 0.00750 |
| 480 | 0.00049 |
| 481 | 0.00407 |
| 482 | 0.00126 |
| 483 | 0.00016 |
| 484 | 0.01947 |
| 485 | 0.02667 |
| 486 | 0.01456 |
| 487 | 0.00393 |
| 488 | 0.00441 |
| 489 | 0.00328 |
| 490 | 0.00669 |
| 491 | 0.00678 |
| 492 | 0.01109 |
| 493 | 0.00128 |
| 494 | 0.00234 |
| 495 | 0.00098 |
| 496 | 0.00934 |
| 497 | 0.08395 |
| 498 | 0.00169 |
| 499 | 0.00438 |
| 500 | 0.03960 |
| 501 | 0.00674 |
| 502 | 0.01215 |
| 503 | 0.00153 |
| 504 | 0.01386 |
| 505 | 0.03233 |
| 506 | 0.07561 |
| 507 | 0.00051 |
| 508 | 0.65071 |
| 509 | 0.04092 |
| 510 | 0.00457 |
| 511 | 0.16757 |
| 512 | 0.35622 |
| 513 | 2.37082 |
| 514 | 0.08857 |
| 515 | 0.09582 |
| 516 | 0.69870 |
| 517 | 0.29898 |
| 518 | 0.61000 |
| 519 | 0.15368 |
| 520 | 0.54370 |
| 521 | 0.37855 |
| 522 | 0.84886 |
| 523 | 0.39282 |
| 524 | 0.07433 |
| 525 | 0.69810 |
| 526 | 0.35365 |
| 527 | 0.05092 |
| 528 | 0.24504 |
| 529 | 0.18932 |
| 530 | 0.35009 |
| 531 | 1.29939 |
| 532 | 0.13094 |
| 533 | 0.81348 |
| 534 | 1.75662 |
| 535 | 0.16978 |
| 536 | 0.01386 |
| 537 | 0.08030 |
| 538 | 0.38146 |
| 539 | 0.17599 |

In one aspect, the present invention provides compounds of formula (I) and their pharmaceutically acceptable salts or esters as described herein, wherein said compounds of formula (I) and their pharmaceutically acceptable salts or esters have IC₅₀'s for MAGL inhibition below 25 μM, preferably below 10 μM, more preferably below 5 μM as measured in the MAGL assay described herein.

In one embodiment, compounds of formula (I) and their pharmaceutically acceptable salts or esters as described herein have IC₅₀ (MAGL inhibition) values between 0.000001 μM and 25 μM, particular compounds have IC₅₀ values between 0.000005 μM and 10 μM, further particular compounds have IC₅₀ values between 0.00005 μM and 5 μM, as measured in the MAGL assay described herein.

Using the Compounds of the Invention

In one aspect, the present invention provides compounds of formula (I), or pharmaceutically acceptable salts thereof, as described herein for use as therapeutically active substance.

In a further aspect, the present invention provides the use of compounds of formula (I), or pharmaceutically acceptable salts thereof, as described herein for the treatment or prophylaxis of neuroinflammation, neurodegenerative diseases, pain, cancer, mental disorders and/or inflammatory bowel disease in a mammal.

In one embodiment, the present invention provides the use of compounds of formula (I), or pharmaceutically acceptable salts thereof, as described herein for the treatment or prophylaxis of neuroinflammation and/or neurodegenerative diseases in a mammal.

In one embodiment, the present invention provides the use of compounds of formula (I), or pharmaceutically acceptable salts thereof, as described herein for the treatment or prophylaxis of neurodegenerative diseases in a mammal.

In one embodiment, the present invention provides the use of compounds of formula (I), or pharmaceutically acceptable salts thereof, as described herein for the treatment or prophylaxis of cancer in a mammal.

In one embodiment, the present invention provides the use of compounds of formula (I), or pharmaceutically acceptable salts thereof, as described herein for the treatment or prophylaxis of inflammatory bowel disease in a mammal.

In one embodiment, the present invention provides the use of compounds of formula (I), or pharmaceutically acceptable salts thereof, as described herein for the treatment or prophylaxis of pain in a mammal.

In one aspect, the present invention provides the use of compounds of formula (I), or pharmaceutically acceptable salts thereof, as described herein for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

In a preferred embodiment, the present invention provides the use of compounds of formula (I), or pharmaceutically acceptable salts thereof, as described herein for the treatment or prophylaxis of multiple sclerosis, Alzheimer's disease and/or Parkinson's disease in a mammal.

In a particularly preferred embodiment, the present invention provides the use of compounds of formula (I), or pharmaceutically acceptable salts thereof, as described herein for the treatment or prophylaxis of multiple sclerosis in a mammal.

In one aspect, the present invention provides compounds of formula (I), or pharmaceutically acceptable salts thereof, as described herein for use in the treatment or prophylaxis of neuroinflammation, neurodegenerative diseases, pain, cancer, mental disorders and/or inflammatory bowel disease in a mammal.

In one embodiment, the present invention provides compounds of formula (I), or pharmaceutically acceptable salts thereof, as described herein for use in the treatment or prophylaxis of neuroinflammation and/or neurodegenerative diseases in a mammal.

In one embodiment, the present invention provides compounds of formula (I), or pharmaceutically acceptable salts thereof, as described herein for use in the treatment or prophylaxis of cancer in a mammal.

In one embodiment, the present invention provides compounds of formula (I), or pharmaceutically acceptable salts thereof, as described herein for use in the treatment or prophylaxis of neurodegenerative diseases in a mammal.

In one embodiment, the present invention provides compounds of formula (I), or pharmaceutically acceptable salts thereof, as described herein for use in the treatment or prophylaxis of inflammatory bowel disease in a mammal.

In one embodiment, the present invention provides compounds of formula (I), or pharmaceutically acceptable salts thereof, as described herein for use in the treatment or prophylaxis of pain in a mammal.

In one aspect, the present invention provides compounds of formula (I), or pharmaceutically acceptable salts thereof, as described herein for use in the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

In a preferred embodiment, the present invention provides compounds of formula (I), or pharmaceutically acceptable salts thereof, as described herein for use in the treatment or prophylaxis of multiple sclerosis, Alzheimer's disease and/or Parkinson's disease in a mammal.

In a particularly preferred embodiment, the present invention provides compounds of formula (I), or pharmaceutically acceptable salts thereof, as described herein for use in the treatment or prophylaxis of multiple sclerosis in a mammal.

In one aspect, the present invention provides the use of compounds of formula (I), or pharmaceutically acceptable salts thereof, as described herein for the preparation of a medicament for the treatment or prophylaxis of neuroinflammation, neurodegenerative diseases, pain, cancer, mental disorders and/or inflammatory bowel disease in a mammal.

In one embodiment, the present invention provides the use of compounds of formula (I), or pharmaceutically acceptable salts thereof, as described herein for the preparation of a medicament for the treatment or prophylaxis of neuroinflammation and/or neurodegenerative diseases in a mammal.

In one embodiment, the present invention provides the use of compounds of formula (I), or pharmaceutically acceptable salts thereof, as described herein for the preparation of a medicament for the treatment or prophylaxis of neurodegenerative diseases in a mammal.

In one embodiment, the present invention provides the use of compounds of formula (I), or pharmaceutically acceptable salts thereof, as described herein for the preparation of a medicament for the treatment or prophylaxis of cancer in a mammal.

In one embodiment, the present invention provides the use of compounds of formula (I), or pharmaceutically acceptable salts thereof, as described herein for the preparation of a medicament for the treatment or prophylaxis of inflammatory bowel disease in a mammal.

In one embodiment, the present invention provides the use of compounds of formula (I), or pharmaceutically acceptable salts thereof, as described herein for the preparation of a medicament for the treatment or prophylaxis of pain in a mammal.

In a further aspect, the present invention provides the use of compounds of formula (I), or pharmaceutically acceptable salts thereof, as described herein for the preparation of a medicament for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

In a preferred embodiment, the present invention provides the use of compounds of formula (I), or pharmaceutically acceptable salts thereof, as described herein for the preparation of a medicament for the treatment or prophylaxis of multiple sclerosis, Alzheimer's disease and/or Parkinson's disease in a mammal.

In a particularly preferred embodiment, the present invention provides the use of compounds of formula (I), or pharmaceutically acceptable salts thereof, as described herein for the preparation of a medicament for the treatment or prophylaxis of multiple sclerosis in a mammal.

In one aspect, the present invention provides a method for the treatment or prophylaxis of neuroinflammation, neurodegenerative diseases, pain, cancer, mental disorders and/or inflammatory bowel disease in a mammal, which method comprises administering an effective amount of a compound of formula (I), or a pharmaceutically acceptable salt thereof, as described herein to the mammal.

In one embodiment, the present invention provides a method for the treatment or prophylaxis of neuroinflammation and/or neurodegenerative diseases in a mammal, which method comprises administering an effective amount of a compound of formula (I), or a pharmaceutically acceptable salt thereof, as described herein to the mammal.

In one embodiment, the present invention provides a method for the treatment or prophylaxis of neurodegenerative diseases in a mammal, which method comprises administering an effective amount of a compound of formula (I), or a pharmaceutically acceptable salt thereof, as described herein to the mammal.

In one embodiment, the present invention provides a method for the treatment or prophylaxis of cancer in a mammal, which method comprises administering an effective amount of a compound of formula (I), or a pharmaceutically acceptable salt thereof, as described herein to the mammal.

In one embodiment, the present invention provides a method for the treatment or prophylaxis of inflammatory bowel disease in a mammal, which method comprises administering an effective amount of a compound of formula (I), or a pharmaceutically acceptable salt thereof, as described herein to the mammal.

In one embodiment, the present invention provides a method for the treatment or prophylaxis of pain in a mammal, which method comprises administering an effective amount of a compound of formula (I), or a pharmaceutically acceptable salt thereof, as described herein to the mammal.

In a further aspect, the present invention provides a method for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal, which method comprises administering an effective amount of a compound of formula (I), or a pharmaceutically acceptable salt thereof, as described herein to the mammal.

In a preferred embodiment, the present invention provides a method for the treatment or prophylaxis of multiple sclerosis, Alzheimer's disease and/or Parkinson's disease in a mammal, which method comprises administering an effective amount of a compound of formula (I), or a pharmaceutically acceptable salt thereof, as described herein to the mammal.

In a particularly preferred embodiment, the present invention provides a method for the treatment or prophylaxis of multiple sclerosis in a mammal, which method comprises administering an effective amount of a compound of formula (I), or a pharmaceutically acceptable salt thereof, as described herein to the mammal.

Pharmaceutical Compositions and Administration

In one aspect, the present invention provides a pharmaceutical composition comprising a compound of formula (I) as described herein and a therapeutically inert carrier.

In one embodiment, there is provided a pharmaceutical composition according to Example 540 or 541.

The compounds of formula (I) and their pharmaceutically acceptable salts and esters can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions).

The compounds of formula (I) and their pharmaceutically acceptable salts and esters can be processed with pharmaceutically inert, inorganic or organic adjuvants for the production of tablets, coated tablets, dragées and hard gelatin capsules. Lactose, corn starch or derivatives thereof, talc, stearic acid or its salts etc. can be used, for example, as such adjuvants for tablets, dragées and hard gelatin capsules.

Suitable adjuvants for soft gelatin capsules are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should be appropriate. It will, however, be clear that the upper limit given herein can be exceeded when this is shown to be indicated.

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC) or crystallization.

All reaction examples and intermediates were prepared under an argon atmosphere if not specified otherwise.

### Example 1

### (6-(3-cyclopropyl-1H-1,2,4-triazol-1-yl)-2-azaspiro[3.3]heptan-2-yl)(3-(4-(1-(trifluoromethyl)cyclopropyl)phenyl)azetidin-1-yl)methanone

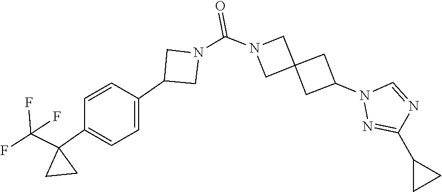 [SMILES: O=C(N1CC(c2ccc(C3(C(F)(F)F)CC3)cc2)C1)N1CC2(CC(n3cnc(C4CC4)n3)C2)C1]

To a solution of (6-(3-cyclopropyl-1H-1,2,4-triazol-1-yl)-2-azaspiro[3.3]heptan-2-yl)(1H-1,2,4-triazol-1-yl)methanone (180 mg, 601 μmol,) in dry DMF (3 mL) was added 3-(4-(1-(trifluoromethyl)cyclopropyl)phenyl)azetidine 4-methylbenzenesulfonate (B.1) (261 mg, 631 μmol) and DIPEA (233 mg, 315 μL, 1.8 mmol) after which the reaction mixture was stirred at 80° C. for 18 h. The crude reaction mixture was directly submitted for reversed-phase HPLC purification to yield 232 mg of the desired product. MS (ESI): m/z=472.3 [M+H]⁺

### Step a) (6-(3-cyclopropyl-]H-1, 2,4-triazol-1-yl)-2-azaspiro[3.3]heptan-2-yl)(]H-1, 2,4-triazol-1-yl)methanone

To a suspension of 6-(3-cyclopropyl-1H-1,2,4-triazol-1-yl)-2-azaspiro[3.3]heptane 2,2,2-trifluoroacetate (A.1) (10.0 g, 31.4 mmol) in dry CH₂Cl₂ (135 mL) cooled down to 0° C. was added DIPEA (12.2 g, 16.5 ml, 94.3 mmol) followed by addition of di(1H-1,2,4-triazol-1-yl)methanone (5.41 g, 33.0 mmol). The reaction mixture was stirred at 0° C. for 5 min and at r.t. for 30 min. The reaction mixture was diluted with dichloromethane and extracted with aq. Na₂CO₃ (1 M solution), the organic phase was collected, dried over sodium sulfate and evaporated down to dryness to yield the (9.27 g), crude). The batch which was used directly without further purification. MS (ESI): m/z=300.2 [M+H]⁺

In analogy to Example 1, Examples in the following table were generated, using the respective building blocks A.X and B.X.

| | | | | MS, |
|---|---|---|---|---|
| | | | Building | ESI: |
| Ex. | Structure | Systematic Name | Blocks | m/z |
| 2 | 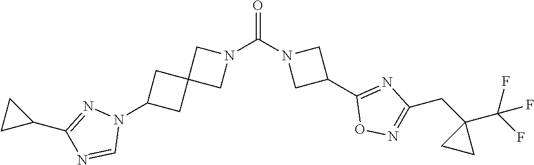 [SMILES: O=C(N1CC(c2nc(CC3(C(F)(F)F)CC3)no2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[3-[[1- (trifluoromethyl)cyclo- propyl]methyl]-1,2,4- oxadiazol-5-yl]azetidin- 1-yl]methanone | A.1 and B.2 | 478.3 [M + H]⁺ |
| 41 | 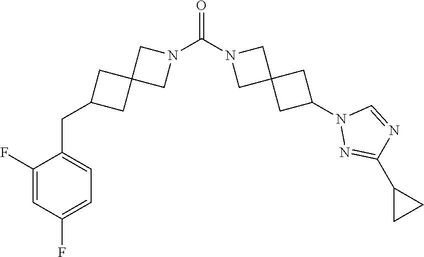 [SMILES: O=C(N1CC2(CC(Cc3ccc(F)cc3F)C2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[6-[(2,4- difluorophenyl)methyl]- 2-azaspiro[3.3]heptan- 2-yl]methanone | A.1 and B.9 | 454.3 [M + H]⁺ |
| 43 | 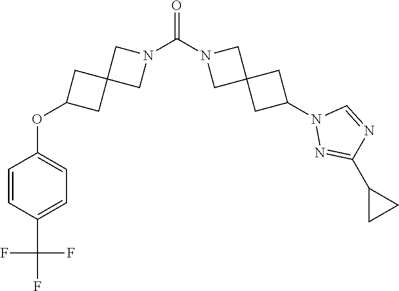 [SMILES: O=C(N1CC2(CC(Oc3ccc(C(F)(F)F)cc3)C2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[6-[4- (trifluoromethyl)phenoxy]- 2- azaspiro[3.3]heptan-2- yl]methanone | A.1 and B.10 | 488.3 [M + H]⁺ |
| 45 | 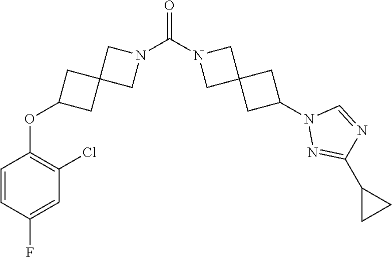 [SMILES: O=C(N1CC2(CC(Oc3ccc(F)cc3Cl)C2)C1)N1CC2(CC(n3cnc(C4CC4)n3)C2)C1] | [6-(2-chloro-4-fluoro- phenoxy)-2- azaspiro[3.3]heptan-2- yl]-[6-(3-cyclopropyl- 1,2,4-triazol-1-yl)-2- azaspiro[3.3]heptan-2- yl]methanone | A.1 and B.12 | 472.4 [M + H]⁺ |
| 46 | 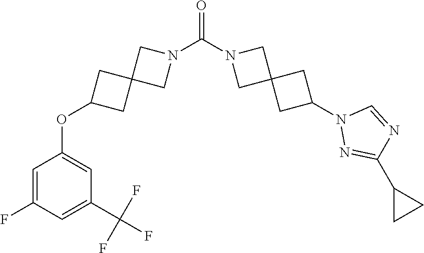 [SMILES: O=C(N1CC2(CC(Oc3cc(F)cc(C(F)(F)F)c3)C2)C1)N1CC2(CC(n3cnc(C4CC4)n3)C2)C1] | (6-(3-cyclopropyl-1H- 1,2,4-triazol-1-yl)-2- azaspiro[3.3]heptan-2- yl)(6-(3-fluoro-5- (trifluoromethyl)phenoxy)- 2- azaspiro[3.3]heptan-2- yl)methanone | A.1 and B.13 | 506.4 [M + H]⁺ |
| 50 | 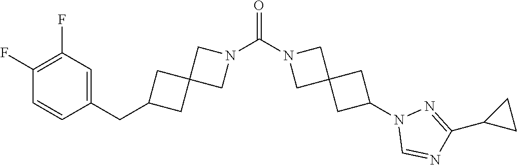 [SMILES: O=C(N1CC2(CC(Cc3ccc(F)c(F)c3)C2)C1)N1CC2(CC(n3cnc(C4CC4)n3)C2)C1] | (6-(3-cyclopropyl-1H- 1,2,4-triazol-1-yl)-2- azaspiro[3.3]heptan-2- yl)(6-(3,4- difluorobenzyl)-2- azaspiro[3.3]heptan-2- yl)methanone | A.1 and B.14 | 454.2 [M + H]⁺ |
| 53 | 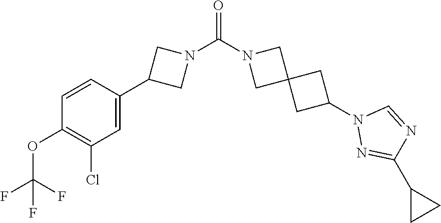 [SMILES: O=C(N1CC(c2ccc(OC(F)(F)F)c(Cl)c2)C1)N1CC2(CC(n3cnc(C4CC4)n3)C2)C1] | [3-[3-chloro-4- (trifluoromethoxy)phe- nyl]azetidin-1-yl]-[6-(3- cyclopropyl-1,2,4- triazol-1-yl)-2- azaspiro[3.3]heptan-2- yl]methanone | A.1 and B.15 | 482.3 [M + H]⁺ |
| 54 | 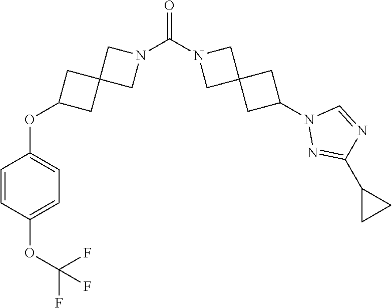 [SMILES: O=C(N1CC2(CC(Oc3ccc(OC(F)(F)F)cc3)C2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[6-[4- (trifluoromethoxy)phe- noxy]-2- azaspiro[3.3]heptan-2- yl]methanone | A.1 and B.16 | 504.4 [M + H]⁺ |
| 59 | 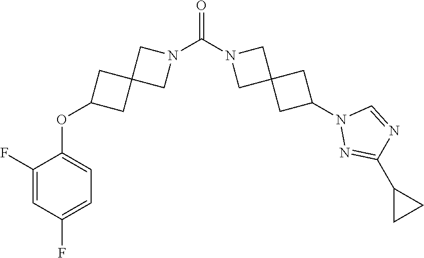 [SMILES: O=C(N1CC2(CC(Oc3ccc(F)cc3F)C2)C1)N1CC2(CC(n3cnc(C4CC4)n3)C2)C1] | (6-(3-cyclopropyl-1H- 1,2,4-triazol-1-yl)-2- azaspiro[3.3]heptan-2- yl)(6-(2,4- difluorophenoxy)-2- azaspiro[3.3]heptan-2- yl)methanone | A.1 and B.17 | 456.4 [M + H]⁺ |
| 68 | 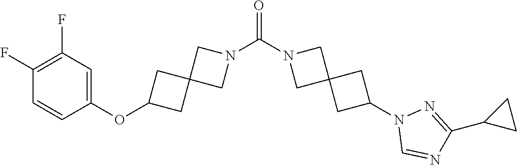 [SMILES: O=C(N1CC2(CC(Oc3ccc(F)c(F)c3)C2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[6-(3,4- difluorophenoxy)-2- azaspiro[3.3]heptan-2- yl]methanone | A.1 and B.18 | 455.2 [M + H]⁺ |
| 71 | 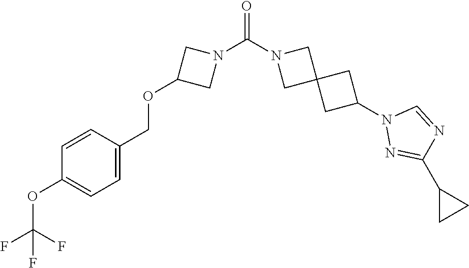 [SMILES: O=C(N1CC(OCc2ccc(OC(F)(F)F)cc2)C1)N1CC2(CC(n3cnc(C4CC4)n3)C2)C1] | (6-(3-cyclopropyl-1H- 1,2,4-triazol-1-yl)-2- azaspiro[3.3]heptan-2- yl)(3-((4- (trifluoromethoxy)ben- zyl)oxy)azetidin-1- yl)methanone | A.1 and B.19 | 478.4 [M + H]⁺ |
| 72 | 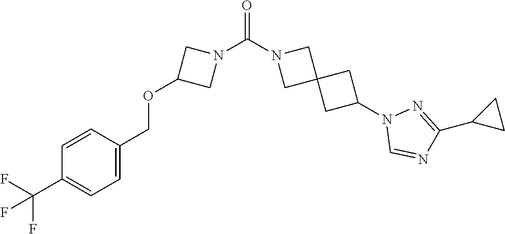 [SMILES: O=C(N1CC(OCc2ccc(C(F)(F)F)cc2)C1)N1CC2(CC(n3cnc(C4CC4)n3)C2)C1] | (6-(3-cyclopropyl-1H- 1,2,4-triazol-1-yl)-2- azaspiro[3.3]heptan-2- yl)(3-((4- (trifluoromethyl)benzyl) oxy)azetidin-1- yl)methanone | A.1 and B.20 | 462.4 [M + H]⁺ |
| 73 | 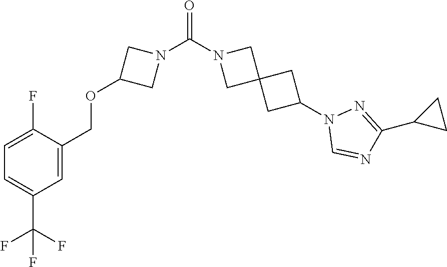 [SMILES: O=C(N1CC(OCc2cc(C(F)(F)F)ccc2F)C1)N1CC2(CC(n3cnc(C4CC4)n3)C2)C1] | (6-(3-cyclopropyl-1H- 1,2,4-triazol-1-yl)-2- azaspiro[3.3]heptan-2- yl)(3-((2-fluoro-5- (trifluoromethyl)benzyl) oxy)azetidin-1- yl)methanone | A.1 and B.21 | 480.4 [M + H]⁺ |
| 74 | 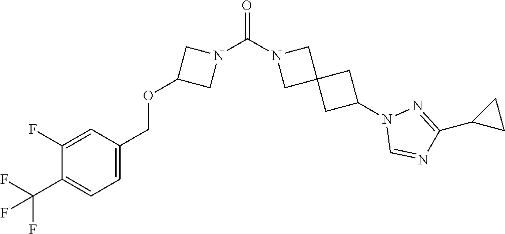 [SMILES: O=C(N1CC(OCc2ccc(C(F)(F)F)c(F)c2)C1)N1CC2(CC(n3cnc(C4CC4)n3)C2)C1] | (6-(3-cyclopropyl-1H- 1,2,4-triazol-1-yl)-2- azaspiro[3.3]heptan-2- yl)(3-((3-fluoro-4- (trifluoromethyl)benzyl) oxy)azetidin-1- yl)methanone | A.1 and B.22 | 480.4 [M + H]⁺ |
| 75 | 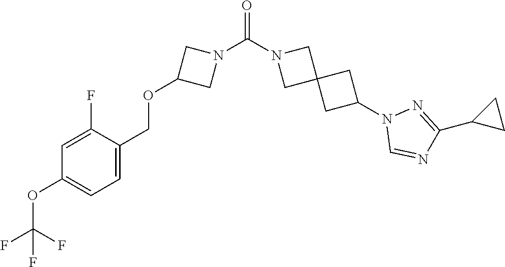 [SMILES: O=C(N1CC(OCc2ccc(OC(F)(F)F)cc2F)C1)N1CC2(CC(n3cnc(C4CC4)n3)C2)C1] | (6-(3-cyclopropyl-1H- 1,2,4-triazol-1-yl)-2- azaspiro[3.3]heptan-2- yl)(3-((2-fluoro-4- (trifluoromethoxy)ben- zyl)oxy)azetidin-1- yl)methanone | A.1 and B.23 | 496.4 [M + H]⁺ |
| 76 | 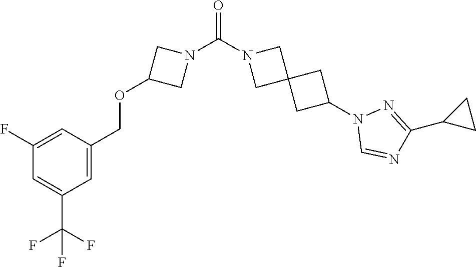 [SMILES: O=C(N1CC(OCc2cc(F)cc(C(F)(F)F)c2)C1)N1CC2(CC(n3cnc(C4CC4)n3)C2)C1] | (6-(3-cyclopropyl-1H- 1,2,4-triazol-1-yl)-2- azaspiro[3.3]heptan-2- yl)(3-((3-fluoro-5- (trifluoromethyl)benzyl) oxy)azetidin-1- yl)methanone | A.1 and B.24 | 480.3 [M + H]⁺ |
| 77 | 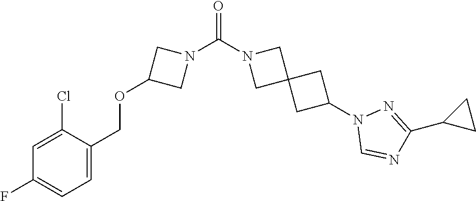 [SMILES: O=C(N1CC(OCc2ccc(F)cc2Cl)C1)N1CC2(CC(n3cnc(C4CC4)n3)C2)C1] | (3-((2-chloro-4- fluorobenzyl)oxy)azetidin- 1-yl)(6-(3- cyclopropyl-1H-1,2,4- triazol-1-yl)-2- azaspiro[3.3]heptan-2- yl)methanone | A.1 and B.25 | 446.4 [M + H]⁺ |
| 78 | 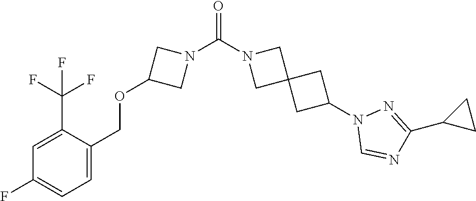 [SMILES: O=C(N1CC(OCc2ccc(F)cc2C(F)(F)F)C1)N1CC2(CC(n3cnc(C4CC4)n3)C2)C1] | (6-(3-cyclopropyl-1H- 1,2,4-triazol-1-yl)-2- azaspiro[3.3]heptan-2- yl)(3-((4-fluoro-2- (trifluoromethyl)benzyl) oxy)azetidin-1- yl)methanone | A.1 and B.26 | 480.4 [M + H]⁺ |
| 79 | 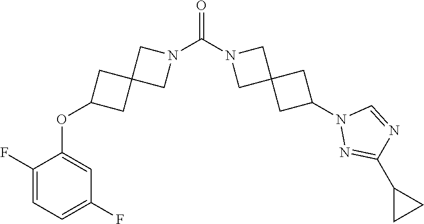 [SMILES: O=C(N1CC2(CC(Oc3cc(F)ccc3F)C2)C1)N1CC2(CC(n3cnc(C4CC4)n3)C2)C1] | (6-(3-cyclopropyl-1H- 1,2,4-triazol-1-yl)-2- azaspiro[3.3]heptan-2- yl)(6-(2,5- difluorophenoxy)-2- azaspiro[3.3]heptan-2- yl)methanone | A.1 and B.27 | 456.3 [M + H]⁺ |
| 80 | 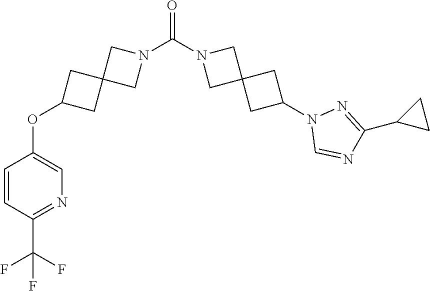 [SMILES: O=C(N1CC2(CC(Oc3ccc(C(F)(F)F)nc3)C2)C1)N1CC2(CC(n3cnc(C4CC4)n3)C2)C1] | (6-(3-cyclopropyl-1H- 1,2,4-triazol-1-yl)-2- azaspiro[3.3]heptan-2- yl)(6-((6- (trifluoromethyl)pyridin- 3-yl)oxy)-2- azaspiro[3.3]heptan-2- yl)methanone | A.1 and B.28 | 489.3 [M + H]⁺ |
| 81 | 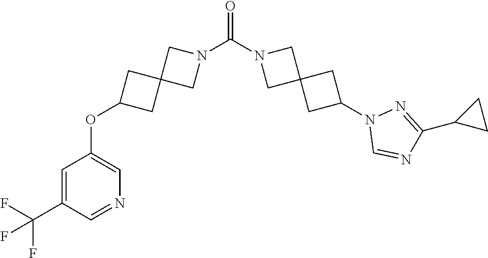 [SMILES: O=C(N1CC2(CC(Oc3cncc(C(F)(F)F)c3)C2)C1)N1CC2(CC(n3cnc(C4CC4)n3)C2)C1] | (6-(3-cyclopropyl-1H- 1,2,4-triazol-1-yl)-2- azaspiro[3.3]heptan-2- yl)(6-((5- (trifluoromethyl)pyridin- 3-yl)oxy)-2- azaspiro[3.3]heptan-2- yl)methanone | A.1 and B.29 | 489.3 [M + H]⁺ |
| 82 | 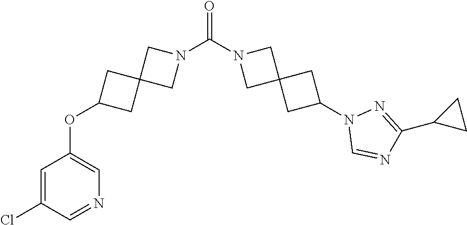 [SMILES: O=C(N1CC2(CC(Oc3cncc(Cl)c3)C2)C1)N1CC2(CC(n3cnc(C4CC4)n3)C2)C1] | (6-((5-chloropyridin-3- yl)oxy)-2- azaspiro[3.3]heptan-2- yl)(6-(3-cyclopropyl- 1H-1,2,4-triazol-1-yl)- 2-azaspiro[3.3]heptan- 2-yl)methanone | A.1 and B.30 | 455.3 [M + H]⁺ |
| 83 | 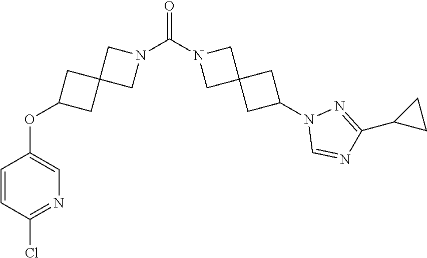 [SMILES: O=C(N1CC2(CC(Oc3ccc(Cl)nc3)C2)C1)N1CC2(CC(n3cnc(C4CC4)n3)C2)C1] | (6-((6-chloropyridin-3- yl)oxy)-2- azaspiro[3.3]heptan-2- yl)(6-(3-cyclopropyl- 1H-1,2,4-triazol-1-yl)- 2-azaspiro[3.3]heptan- 2-yl)methanone | A.1 and B.31 | 455.3 [M + H]⁺ |
| 94 | 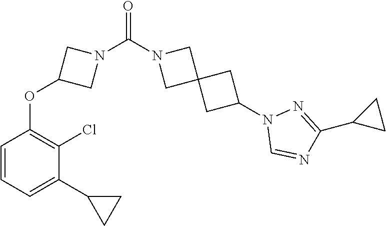 [SMILES: O=C(N1CC(Oc2cccc(C3CC3)c2Cl)C1)N1CC2(CC(n3cnc(C4CC4)n3)C2)C1] | (3-(2-chloro-3- cyclopropylphenoxy) azetidin-1-yl)(6-(3- cyclopropyl-1H-1,2,4- triazol-1-yl)-2- azaspiro[3.3]heptan-2- yl)methanone | A.1 and B.36 | 454.3 [M + H]⁺ |
| 95 | 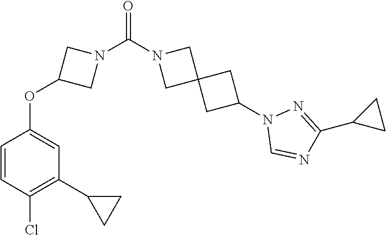 [SMILES: O=C(N1CC(Oc2ccc(Cl)c(C3CC3)c2)C1)N1CC2(CC(n3cnc(C4CC4)n3)C2)C1] | (3-(4-chloro-3- cyclopropylphenoxy) azetidin-1-yl)(6-(3- cyclopropyl-1H-1,2,4- triazol-1-yl)-2- azaspiro[3.3]heptan-2- yl)methanone | A.1 and B.37 | 454.3 [M + H]⁺ |
| 96 | 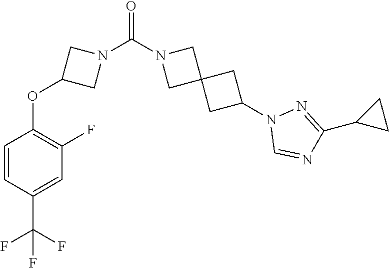 [SMILES: O=C(N1CC(Oc2ccc(C(F)(F)F)cc2F)C1)N1CC2(CC(n3cnc(C4CC4)n3)C2)C1] | (6-(3-cyclopropyl-1H- 1,2,4-triazol-1-yl)-2- azaspiro[3.3]heptan-2- yl)(3-(2-fluoro-4- (trifluoromethyl)phenoxy) azetidin-1- yl)methanone | A.1 and B.39 | 466.3 [M + H]⁺ |
| 97 | 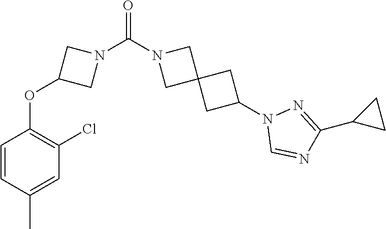 [SMILES: Cc1ccc(OC2CN(C(=O)N3CC4(CC(n5cnc(C6CC6)n5)C4)C3)C2)c(Cl)c1] | (3-(2-chloro-4- methylphenoxy)azetidin- 1-yl)(6-(3-cyclopropyl- 1H-1,2,4-triazol-1-yl)- 2-azaspiro[3.3]heptan- 2-yl)methanone | A.1 and B.41 | 428.3 [M + H] |
| 98 | 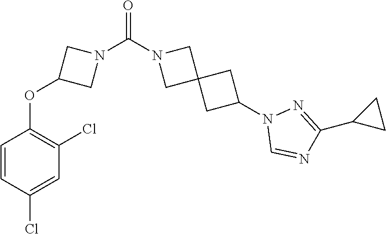 [SMILES: O=C(N1CC(Oc2ccc(Cl)cc2Cl)C1)N1CC2(CC(n3cnc(C4CC4)n3)C2)C1] | (6-(3-cyclopropyl-1H- 1,2,4-triazol-1-yl)-2- azaspiro[3.3]heptan-2- yl)(3-(2,4- dichlorophenoxy)azetidin- 1-yl)methanone | A.1 and B.42 | 448.2 [M + H]⁺ |
| 99 | 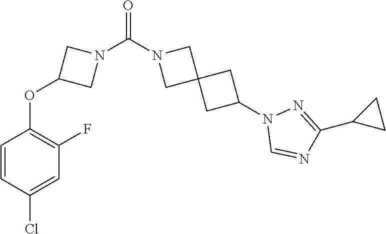 [SMILES: O=C(N1CC(Oc2ccc(Cl)cc2F)C1)N1CC2(CC(n3cnc(C4CC4)n3)C2)C1] | (3-(4-chloro-2- fluorophenoxy)azetidin- 1-yl)(6-(3-cyclopropyl- 1H-1,2,4-triazol-1-yl)- 2-azaspiro[3.3]heptan- 2-yl)methanone | A.1 and B.40 | 432.2 [M + H]⁺ |
| 100 | 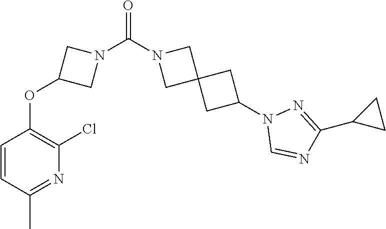 [SMILES: Cc1ccc(OC2CN(C(=O)N3CC4(CC(n5cnc(C6CC6)n5)C4)C3)C2)c(Cl)n1] | (3-((2-chloro-6- methylpyridin-3- yl)oxy)azetidin-1-yl)(6- (3-cyclopropyl-1H- 1,2,4-triazol-1-yl)-2- azaspiro[3.3]heptan-2- yl)methanone | A.1 and B.38 | 429.3 [M + H]⁺ |
| 111 | 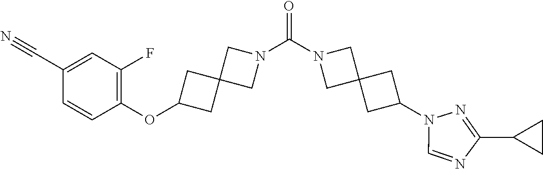 [SMILES: N#Cc1ccc(OC2CC3(C2)CN(C(=O)N2CC4(CC(n5cnc(C6CC6)n5)C4)C2)C3)c(F)c1] | 4-[[2-[6-(3- cyclopropyl-1,2,4- triazol-1-yl)-2- azaspiro[3.3]heptane-2- carbonyl]-2- azaspiro[3.3]heptan-6- yl]oxy]-3-fluoro- benzonitrile | A.1 and B.49 | 463.2 [M + H]⁺ |
| 136 | 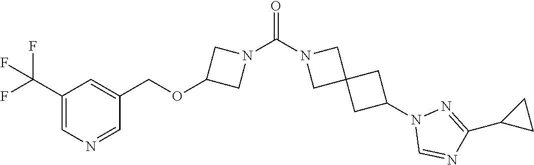 [SMILES: O=C(N1CC(OCc2cncc(C(F)(F)F)c2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[[5- (trifluoromethyl)-3- pyridyl]methoxy]azetidin- 1-yl]methanone | A.1 and B.50 | 463.2 [M + H]⁺ |
| 137 | 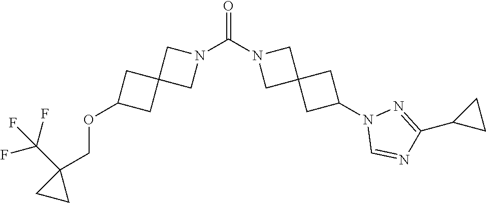 [SMILES: O=C(N1CC2(CC(OCC3(C(F)(F)F)CC3)C2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[6-[[1- (trifluoromethyl)cyclo- propyl]methoxy]-2- azaspiro[3.3]heptan-2- yl]methanone | A.1 and B.51 | 466.3 [M + H]⁺ |
| 138 | 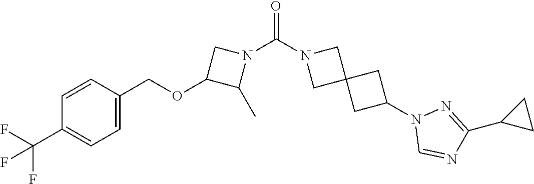 [SMILES: CC1C(OCc2ccc(C(F)(F)F)cc2)CN1C(=O)N1CC2(CC(n3cnc(C4CC4)n3)C2)C1] | [6-(3-cyclopropyl- 1,2,4-triazol-1-yl)-2- azaspiro[3.3]heptan-2- yl]-[2-methyl-3-[[4- (trifluoromethyl)phenyl] methoxy]azetidin-1- yl]methanone | A.1 and B.52 | 476.4 [M + H]⁺ |
| 140 | 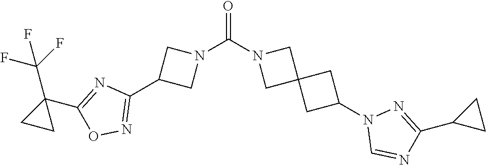 [SMILES: O=C(N1CC(c2noc(C3(C(F)(F)F)CC3)n2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[5-[1- (trifluoromethyl)cyclo- propyl]-1,2,4-oxadiazol- 3-yl]azetidin-1- yl]methanone | A.1 and B.53 | 464.3 [M + H]⁺ |
| 145 | 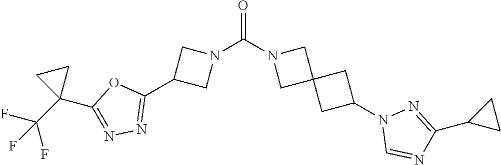 [SMILES: O=C(N1CC(c2nnc(C3(C(F)(F)F)CC3)o2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[5-[1- (trifluoromethyl)cyclo- propyl]-1,3,4-oxadiazol- 2-yl]azetidin-1- yl]methanone | A.1 and B.55 | 464.3 [M + H]⁺ |
| 164 | 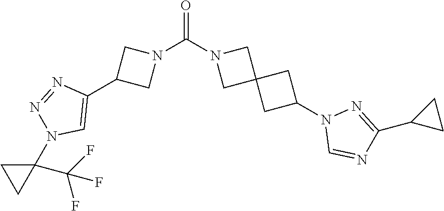 [SMILES: O=C(N1CC(c2cn(C3(C(F)(F)F)CC3)nn2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[1-[1- (trifluoromethyl)cyclo- propyl]triazol-4- yl]azetidin-1- yl]methanone | A.1 and B.58 | 463.3 [M + H]⁺ |
| 166 | 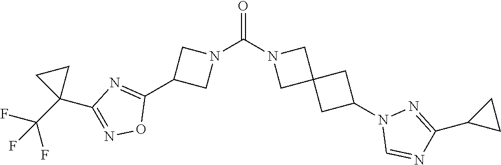 [SMILES: O=C(N1CC(c2nc(C3(C(F)(F)F)CC3)no2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[3-[1- (trifluoromethyl)cyclo- propyl]-1,2,4-oxadiazol- 5-yl]azetidin-1- yl]methanone | A.1 and B.59 | 464.3 [M + H]⁺ |
| 167 | 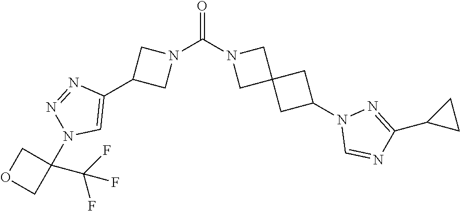 [SMILES: O=C(N1CC(c2cn(C3(C(F)(F)F)COC3)nn2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[1-[3- (trifluoromethyl)oxetan- 3-yl]triazol-4- yl]azetidin-1- yl]methanone | A.1 and B.60 | 479.3 [M + H]⁺ |
| 165 | 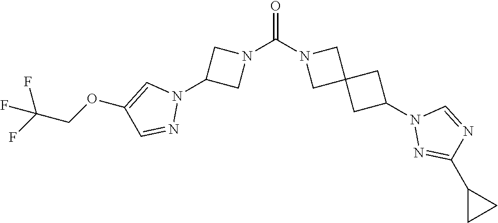 [SMILES: O=C(N1CC(n2cc(OCC(F)(F)F)cn2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[4-(2,2,2- trifluoroethoxy)pyrazol- 1-yl]azetidin-1- yl]methanone | A.1 and B.54 | 452.3 [M + H]⁺ |

### Example 3

### [4-(5-tert-butyl-1,2,4-oxadiazol-3-yl)phenyl]−[6-(3-cyclopropyl-1,2,4-triazol-1-yl)-2-azaspiro[3.3]heptan-2-yl]methanone

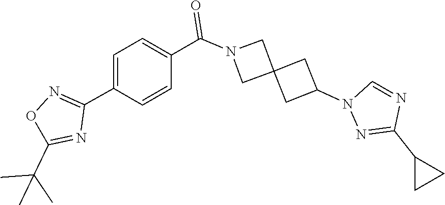 [SMILES: CC(C)(C)c1nc(-c2ccc(C(=O)N3CC4(CC(n5cnc(C6CC6)n5)C4)C3)cc2)no1]

To a mixture of 3-cyclopropyl-1H-1,2,4-triazole (CAS: 1211390-33-8) (21.8 mg, 200 μmol) and [2-[4-(5-tert-butyl-1,2,4-oxadiazol-3-yl)benzoyl]-2-azaspiro[3.3]heptan-6-yl]methanesulfonate (83 mg, 200 μmol) in CH₃CN (2.0 mL) was added Cs₂CO₃ (600 μmol) in one portion in 8 mL vials. The mixture was shaken at 100° C. for 16 h. Filter and purify mixture by prep-HPLC to give the title compound (23.9 mg, 28% yield). MS (ESI): m/z=433.3 [M+H]⁺

### Step a) [4-(5-tert-butyl-1, 2, 4-oxadiazol-3-yl)phenyl]-(6-hydroxy-2-azaspiro[3.3]heptan-2-yl)methanone

To a solution of 4-(5-tert-butyl-1,2,4-oxadiazol-3-yl)benzoic acid (CAS: 1119452-72-0) (3.8 g, 15.4 mmol) and 2-azaspiro[3.3]heptan-6-ol (2.54 g, 17.0 mmol, HCl salt) in THF (100 mL) were added HATU (8.80 g, 23.2 mmol) and TEA (6.25 g, 61.7 mmol, 8.59 mL). The reaction was stirred at 25° C. for 16 h. The solution was filtered and concentrated under reduced pressure at 40° C. The residue was purified by flash silica gel chromatography (eluting with 0 to 40% THF/Petroleum ether gradient). The title compound (3.8 g, 10.0 mmol, 64.9% yield) was obtained as a white solid. MS (ESI): m/z=342.3 [M+H]⁺

### Step b) (2-44-(5-tert-butyl-yl-(,2,4-oxadiazol-3-yl)benzoyl]-2-azaspiro[3.3]heptan-6yl] methanesulfonate

To a solution of [4-(5-tert-butyl-1,2,4-oxadiazol-3-yl)phenyl]-(6-hydroxy-2-azaspiro[3.3]heptan-2-yl)methanone (1.00 g, 2.64 mmol) and TEA (534 mg, 5.27 mmol, 734 μL) in DCM (10 mL) was added MsCl (1.34 g, 11.7 mmol, 905 μL) drop wise at 0° C. The resulting mixture was stirred at 0° C. for 2 h. The residue was poured into water (20 mL). The aqueous phase was extracted with DCM (20 mL×3). The combined organic phase was dried with anhydrous Na₂SO₄, filtered and concentrated in vacuum. The title compound (1.5 g, crude) was obtained as yellow oil, and was used for next step directly without further purification. MS (ESI): m/z=420.2 [M+H]⁺

In analogy to Example 3, Examples in the following table were generated, using the respective heteroarene building blocks in the final step. In some cases NaOtBu was used in place of Cs₂CO₃ in the final step.

| | | | | MS, |
|---|---|---|---|---|
| | | | Building | ESI: |
| Ex. | Structure | Systematic Name | Blocks | m/z |
| 4 | 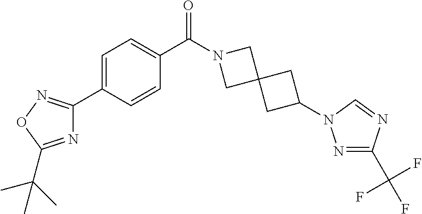 [SMILES: CC(C)(C)c1nc(-c2ccc(C(=O)N3CC4(CC(n5cnc(C(F)(F)F)n5)C4)C3)cc2)no1] | (4-(5-(tert-butyl)-1,2,4- oxadiazol-3- yl)phenyl)(6-(3- (trifluoromethyl)-1H- 1,2,4-triazol-1-yl)-2- azaspiro[3.3]heptan-2- yl)methanone | 3- (trifluoro methyl)- 1H- 1,2,4- triazole (CAS: 60406- 75-9) | 461.3 [M + H]⁺ |
| 5 | 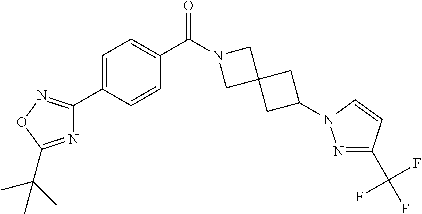 [SMILES: CC(C)(C)c1nc(-c2ccc(C(=O)N3CC4(CC(n5ccc(C(F)(F)F)n5)C4)C3)cc2)no1] | [4-(5-tert-butyl-1,2,4- oxadiazol-3-yl)phenyl]- [6-[3- (trifluoromethyl) pyrazol-1-yl]-2- azaspiro[3.3]heptan-2- yl]methanone | 3- (trifluoro methyl)- 1H- pyrazole (CAS: 20154- 03-4) | 460.3 [M + H]⁺ |
| 6 | 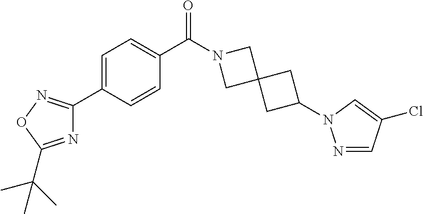 [SMILES: CC(C)(C)c1nc(-c2ccc(C(=O)N3CC4(CC(n5cc(Cl)cn5)C4)C3)cc2)no1] | [4-(5-tert-butyl-1,2,4- oxadiazol-3-yl)phenyl]- [6-(4-chloropyrazol-1- yl)-2- azaspiro[3.3]heptan-2- yl]methanone | 4-chloro- pyrazole (CAS: 15878- 00-9) | 426.2 [M + H]⁺ |
| 7 | 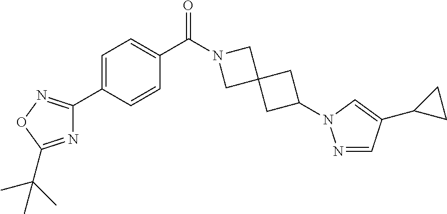 [SMILES: CC(C)(C)c1nc(-c2ccc(C(=O)N3CC4(CC(n5cc(C6CC6)cn5)C4)C3)cc2)no1] | [4-(5-tert-butyl-1,2,4- oxadiazol-3-yl)phenyl]- [6-(4- cyclopropylpyrazol-1- yl)-2- azaspiro[3.3]heptan-2- yl]methanone | 4- cyclopro- pyl- pyrazole (CAS: 90253- 21-7) | 432.3 [M + H]⁺ |
| 8 | 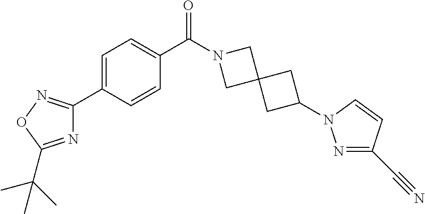 [SMILES: CC(C)(C)c1nc(-c2ccc(C(=O)N3CC4(CC(n5ccc(C#N)n5)C4)C3)cc2)no1] | 1-[2-[4-(5-tert-butyl- 1,2,4-oxadiazol-3- yl)benzoyl]-2- azaspiro[3.3]heptan-6- yl]pyrazole-3- carbonitrile | 1H- pyrazole- 3-carbon- itrile (CAS: 36650- 74-5) | 417.3 [M + H]⁺ |
| 9 | 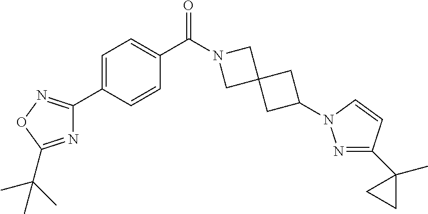 [SMILES: CC(C)(C)c1nc(-c2ccc(C(=O)N3CC4(CC(n5ccc(C6(C)CC6)n5)C4)C3)cc2)no1] | [4-(5-tert-butyl-1,2,4- oxadiazol-3-yl)phenyl]- [6-[3-(1- methylcyclopropyl) pyrazol-1-yl]-2- azaspiro[3.3]heptan-2- yl]methanone | 3-(1- methyl- cyclopro- pyl)- 1(2)H- pyrazole (CAS: 18712- 28-2) | 446.4 [M + H]⁺ |
| 10 | 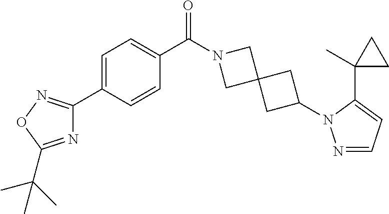 [SMILES: CC(C)(C)c1nc(-c2ccc(C(=O)N3CC4(CC(n5nccc5C5(C)CC5)C4)C3)cc2)no1] | [4-(5-tert-butyl-1,2,4- oxadiazol-3-yl)phenyl]- [6-[5-(1- methylcyclopropyl) pyrazol-1-yl]-2- azaspiro[3.3]heptan-2- yl]methanone | 3-(1- methyl- cyclopro- pyl)- 1(2)H- pyrazole (CAS: 18712- 28-2) | 446.4 [M + H]⁺ |
| | (regioisomeric sideproduct, | | | |
| | generated with Example 9) | | | |
| 11 | 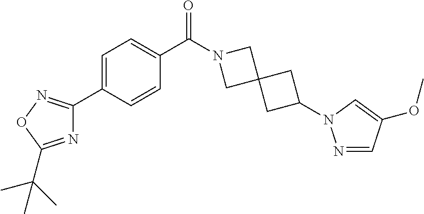 [SMILES: COc1cnn(C2CC3(C2)CN(C(=O)c2ccc(-c4noc(C(C)(C)C)n4)cc2)C3)c1] | [4-(5-tert-butyl-1,2,4- oxadiazol-3-yl)phenyl]- [6-(4-methoxypyrazol- 1-yl)-2- azaspiro[3.3]heptan-2- yl]methanone | 4- methoxy- pyrazole (CAS: 14884- 01-6) | 422.3 [M + H]⁺ |
| 12 | 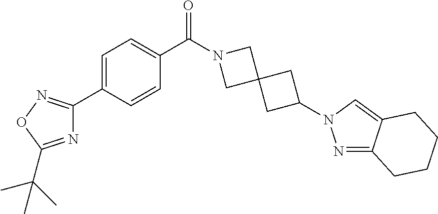 [SMILES: CC(C)(C)c1nc(-c2ccc(C(=O)N3CC4(CC(n5cc6c(n5)CCCC6)C4)C3)cc2)no1] | [4-(5-tert-butyl-1,2,4- oxadiazol-3-yl)phenyl]- [6-(4,5,6,7- tetrahydroindazol-2-yl)- 2-azaspiro[3.3]heptan- 2-yl]methanone | 4,5,6,7- tetrahy- dro-2H- indazole (CAS: 15409- 56-0) | 446.3 [M + H]⁺ |
| 13 | 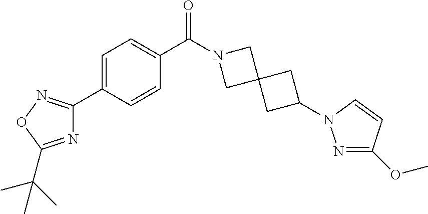 [SMILES: COc1ccn(C2CC3(C2)CN(C(=O)c2ccc(-c4noc(C(C)(C)C)n4)cc2)C3)n1] | [4-(5-tert-butyl-1,2,4- oxadiazol-3-yl)phenyl]- [6-(3-methoxypyrazol- 1-yl)-2- azaspiro[3.3]heptan-2- yl]methanone | 3- methoxy- 1H- pyrazole (CAS: 215610- 30-3) | 422.3 [M + H]⁺ |
| 14 | 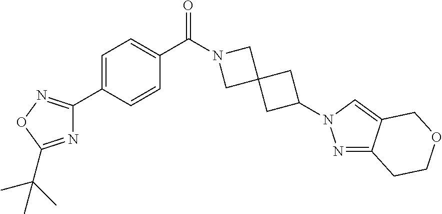 [SMILES: CC(C)(C)c1nc(-c2ccc(C(=O)N3CC4(CC(n5cc6c(n5)CCOC6)C4)C3)cc2)no1] | [4-(5-tert-butyl-1,2,4- oxadiazol-3-yl)phenyl]- [6-(6,7-dihydro-4H- pyrano[4,3-c]pyrazol-2- yl)-2- azaspiro[3.3]heptan-2- yl]methanone | 2,4,6,7- tetrahy- dro- pyrano [4,3-c] pyrazole (CAS: 258353- 57-0) | 448.3 [M + H]⁺ |
| 15 | 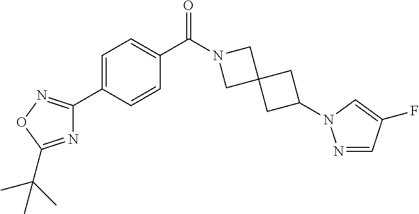 [SMILES: CC(C)(C)c1nc(-c2ccc(C(=O)N3CC4(CC(n5cc(F)cn5)C4)C3)cc2)no1] | [4-(5-tert-butyl-1,2,4- oxadiazol-3-yl)phenyl]- [6-(4-fluoropyrazol-1- yl)-2- azaspiro[3.3]heptan-2- yl]methanone | 4-fluoro- 1H- pyrazole (CAS: 35277- 02-2) | 410.3 [M + H]⁺ |
| 16 | 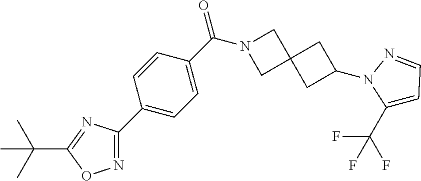 [SMILES: CC(C)(C)c1nc(-c2ccc(C(=O)N3CC4(CC(n5nccc5C(F)(F)F)C4)C3)cc2)no1] | [4-(5-tert-buty1-1,2,4- oxadiazol-3-yl)phenyl]- [6-[5- (trifluoromethyl) pyrazol-1-yl]-2- azaspiro[3.3]heptan-2- yl]methanone | 3- (trifluoro methyl)- 1H- pyrazole (CAS: 20154- 03-4) | 460.3 [M + H]⁺ |
| | (regioisomeric sideproduct, | | | |
| | generated with Example 5) | | | |
| 17 | 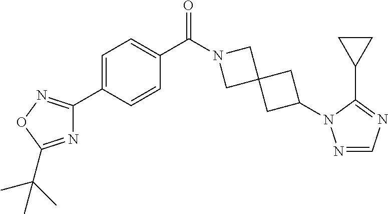 [SMILES: CC(C)(C)c1nc(-c2ccc(C(=O)N3CC4(CC(n5ncnc5C5CC5)C4)C3)cc2)no1] | [4-(5-tert-butyl-1,2,4- oxadiazol-3-yl)phenyl]- [6-(5-cyclopropyl- 1,2,4-triazol-1-yl)-2- azaspiro[3.3]heptan-2- yl]methanone | 3- cyclopro- pyl-1H- 1,2,4- triazole (CAS: 1211390- 33-8) | 433.3 [M + H]⁺ |
| | (regioisomeric sideproduct, | | | |
| | generated with Example 3) | | | |
| 18 | 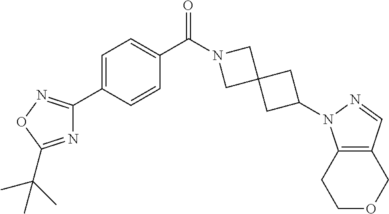 [SMILES: CC(C)(C)c1nc(-c2ccc(C(=O)N3CC4(CC(n5ncc6c5CCOC6)C4)C3)cc2)no1] | [4-(5-tert-butyl-1,2,4- oxadiazol-3-yl)phenyl]- [6-(6,7-dihydro-4H- pyrano[4,3-c]pyrazol-1- yl)-2- azaspiro[3.3]heptan-2- yl]methanone | 2,4,6,7- tetrahy- dro- pyrano [4,3-c] pyrazole (CAS: 258353- 57-0) | 448.3 [M + H]⁺ |
| | (regioisomeric sideproduct, | | | |
| | generated with Example 14) | | | |
| 19 | 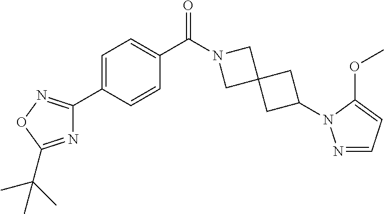 [SMILES: COc1ccnn1C1CC2(C1)CN(C(=O)c1ccc(-c3noc(C(C)(C)C)n3)cc1)C2] | [4-(5-tert-butyl-1,2,4- oxadiazol-3-yl)phenyl]- [6-(5-methoxypyrazol- 1-yl)-2- azaspiro[3.3]heptan-2- yl]methanone | 3- methoxy- 1H- pyrazole (CAS: 215610- 30-3) | 422.3 [M + H]⁺ |
| | (regioisomeric sideproduct, | | | |
| | generated with Example 13) | | | |
| 22 | 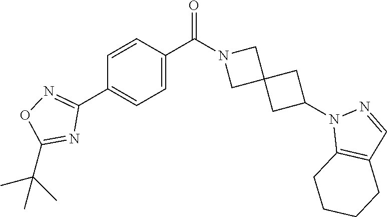 [SMILES: CC(C)(C)c1nc(-c2ccc(C(=O)N3CC4(CC(n5ncc6c5CCCC6)C4)C3)cc2)no1] | [4-(5-tert-butyl-1,2,4- oxadiazol-3-yl)phenyl]- [6-(4,5,6,7- tetrahydroindazol-1-yl)- 2-azaspiro[3.3]heptan- 2-yl]methanone | 4,5,6,7- tetrahy- dro-2H- indazole (CAS: 15409- 56-0) | 446.3 [M + H]⁺ |
| | (regioisomeric sideproduct, | | | |
| | generated with Example 13) | | | |

### Example 20

### 1-[2-[4-(5-tert-butyl-1,2,4-oxadiazol-3-yl)benzoyl]-2-azaspiro[3.3]heptan-6-yl]-1,2,4-triazole-3-carbonitrile

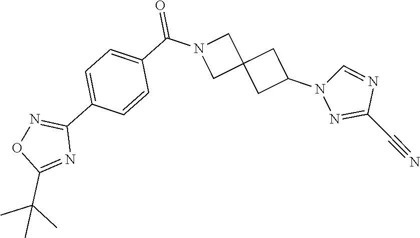 [SMILES: CC(C)(C)c1nc(-c2ccc(C(=O)N3CC4(CC(n5cnc(C#N)n5)C4)C3)cc2)no1]

To a mixture of [4-(5-tert-butyl-1,2,4-oxadiazol-3-yl)phenyl]-(6-hydroxy-2-azaspiro[3.3]heptan-2-yl)methanone (Example 3, Step a) (68 mg, 200 μmol) and 1H-1,2,4-triazole-3-carbonitrile (CAS: 3641-10-9) (18.8 mg, 200 μmol) in THF (2.0 mL) was added PS—PPh₃ (600 μmol) in one portion under N₂ in 8 mL vials. The mixture was added DIAD (260 μmol) at 0° C., and then the vials were capped and shaken at 30° C. for 16 h. The reaction mixture was filtered and solvent concentrated by Speedvac. The residue was purified by prep-HPLC to give the title compound (24.6 mg, 29.4%). MS (ESI): m/z=418.3 [M+H]⁺

In analogy to Example 20, Examples in the following table were generated, using the respective heteroarene building blocks.

| | | | | MS, |
|---|---|---|---|---|
| | | | Building | ESI: |
| Ex. | Structure | Systematic Name | Blocks | m/z |
| 21 | 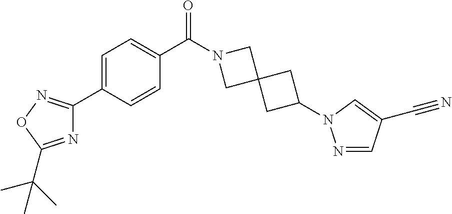 [SMILES: CC(C)(C)c1nc(-c2ccc(C(=O)N3CC4(CC(n5cc(C#N)cn5)C4)C3)cc2)no1] | 1-[2-[4-(5-tert-butyl- 1,2,4-oxadiazol-3- yl)benzoyl]-2- azaspiro[3.3]heptan-6- yl]pyrazole-4- carbonitrile | 1H- pyrazole- 4-car- bonitrile (CAS: 31108- 57-3) | 417.1 [M + H]⁺ |

### Example 23

### [4-(5-tert-butyl-1,2,4-oxadiazol-3-yl)phenyl]−[6-(6-methyl-3-pyridyl)-2-azaspiro[3.3]heptan-2-yl]methanone

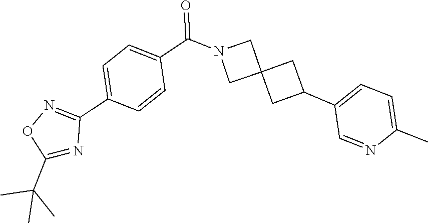 [SMILES: Cc1ccc(C2CC3(C2)CN(C(=O)c2ccc(-c4noc(C(C)(C)C)n4)cc2)C3)cn1]

A mixture of [4-(5-tert-butyl-1,2,4-oxadiazol-3-yl)phenyl]-(6-iodo-2-azaspiro[3.3]heptan-2-yl)methanone (90 mg, 200 μmol), Zn (1 mmol), TMSCl (20 μmol) and 1,2-dibromoethane (20 μmol) in THF (3 mL) was stirred at 65° C. for 1 h under N₂ atmosphere to obtain a solution, which was added dropwise to a mixture of 5-bromo-2-methylpyridine (41 mg, 300 μmol), Pd₂(dba)₃ (10 μmol) and Q-Phos (10 μmol) in THF (2 mL). After dropwise addition was finished, the mixture was at stirred at 30° C. for 2 h. The solvent was removed under reduced pressure. The residue was washed with 1.5 mL of water and extracted with EtOAc (2 mL×3). The organic layers were combined and dried over anhydrous Na₂SO₄, concentrated to give a residue, which was purified by prep-HPLC to give the title compound (11.4 mg, 13.7%). MS (ESI): m/z=418.3 [M+H]⁺

### Step a) [4-(5-tert-butyl-1,2,4-oxadiazol-3-yl)phenyl]-(6-iodo-2-azaspiro[3.3]heptan-2-yl)methanone

To a solution of [4-(5-tert-butyl-1,2,4-oxadiazol-3-yl)phenyl]-(6-hydroxy-2-azaspiro[3.3]heptan-2-yl)methanone (Example 3, Step a) (0.80 g, 2.1 mmol) and 1₂ (803 mg, 3.16 mmol, 637 μL) in toluene (30 mL) were added imidazole (431 mg, 6.33 mmol) and triphenylphosphine (1.11 g, 4.22 mmol). The reaction mixture was stirred at 110° C. for 2 h. The solution was concentrated in reduced pressure at 50° C. The residue was purified by flash silica gel chromatography, eluting with 0-100% DCM/petroleum ether gradient, to obtain the title compound (0.8 g, 1.60 mmol, 75.7% yield) as a white solid. MS (ESI): m/z=452.1 [M+H]⁺

### Example 30

### [[6-(4-cyclopropylimidazol-1-yl)-2-azaspiro[3.3]heptan-2-yl]−[5-fluoro-6-[(1-methylcyclopropyl)methoxy]-3-pyridyl]methanone

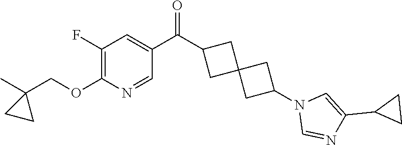 [SMILES: CC1(COc2ncc(C(=O)C3CC4(C3)CC(n3cnc(C5CC5)c3)C4)cc2F)CC1]

To a solution of [6-(4-cyclopropylimidazol-1-yl)-2-azaspiro[3.3]heptan-2-yl]-(5,6-difluoro-3-pyridyl)methanone (crude, 0.12 mmol) and 1-methylcyclopropanemethanol (103 mg, 1.2 mmol), TEA (85.7 μL, 0.6 mmol) in DMSO (1.0 mL) was added CsF (54.6 mg, 0.36 mmol). The mixture was stirred at 110° C. for 2 h under microwave. The mixture was were filtered and concentrated under reduced pressure to give a residue which was purified by prep-HPLC to give the title compound (24.1 mg, 0.052 mmol, 43.3% yield). MS (ESI): m/z=411.2 [M+H]⁺.

### Step a) [6-(4-cyclopropylimidazol-1-yl)-2-azaspiro[3.3]heptan-2-yl]-(5, 6-difluoro-3-pyridyl)methanone

To a solution of 6-(4-cyclopropylimidazol-1-yl)-2-azaspiro[3.3]heptane;2,2,2-trifluoroacetic acid salt (Example A.2) (24.3 mg, 0.12 mmol) and 5,6-difluoronicotinic acid (CAS: 851386-33-9) (19.1 mg, 0.12 mmol), TEA (85.7 μL, 0.6 mmol) in ACN (1.0 mL) was added T3P (78.5 μL, 0.13 mmol). The mixture was stirred at 80° C. for 16 h. The reaction mixture was concentrated under reduced pressure to give a residue, which was diluted with NaOH (1.0 M aq. solution, 0.5 mL), H₂O (3.0 mL) and extracted with EtOAc (3 mL×3). The combined organic layers were dried over Na₂SO₄, filtered and concentrated under reduced pressure to give the title compound, which was directly used without further purification. MS (ESI): m/z=345.2 [M+H]⁺

In analogy to Example 30, Examples in the following table were generated, using the respective building blocks in place of A.2.

| | | | | MS, |
|---|---|---|---|---|
| | | | Building | ESI: |
| Ex. | Structure | Systematic Name | Blocks | m/z |
| 29 | 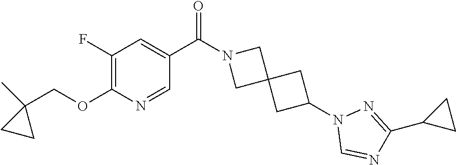 [SMILES: CC1(COc2ncc(C(=O)N3CC4(CC(n5cnc(C6CC6)n5)C4)C3)cc2F)CC1] | [6-(3-cyclopropyl- 1,2,4-triazol-1-yl)-2- azaspiro[3.3]heptan-2- yl]-[5-fluoro-6-[(1- methylcyclopropyl) methoxy]-3- pyridyl]methanone | A.1 | 412.2 [M + H]⁺ |

### Example 26

### [6-(3-cyclopropyl-1,2,4-triazol-1-yl)-2-azaspiro[3.3]heptan-2-yl]−[5-fluoro-6-[[1-(trifluoromethyl)cyclopropyl]methoxy]-3-pyridyl]methanone

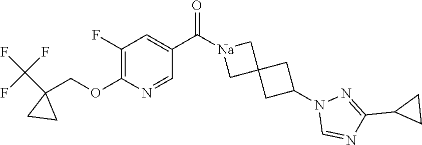

To a solution of 6-(3-cyclopropyl-1H-1,2,4-triazol-1-yl)-2-azaspiro[3.3]heptane (Example A.1) (24.5 mg, 0.12 mmol), 5-fluoro-6-[[1-(trifluoromethyl)cyclopropyl]methoxy]pyridine-3-carboxylic acid (Example B.4) (0.12 mmol) and TEA (85.7 μL, 0.6 mmol) in ACN (1.0 mL) was added T3P (78.5 μL, 0.13 mmol). The mixture was stirred at 80° C. for 16 h. The mixture was concentrated under reduced pressure to give a residue. The residue was purified by prep-HPLC to give the title compound (19.4 mg, 0.042 mmol, 35.0% yield). MS (ESI): m/z=466.2 [M+H]⁺.

In analogy to Example 1, the Examples in the following table were generated, using the respective building blocks A.X and B.X. Other typical coupling agents used include HATU and other typical bases include DIPEA.

| | | | | MS, |
|---|---|---|---|---|
| | | | Building | ESI: |
| Ex. | Structure | Systematic Name | Blocks | m/z |
| 24 | 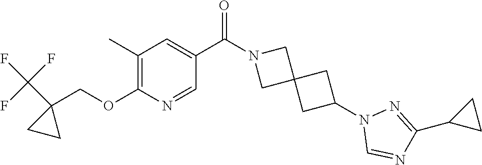 [SMILES: Cc1cc(C(=O)N2CC3(CC(n4cnc(C5CC5)n4)C3)C2)cnc1OCC1(C(F)(F)F)CC1] | [6-(3-cyclopropyl- 1,2,4-triazol-1-yl)-2- azaspiro[3.3]heptan-2- yl]-[5-methyl-6-[[1- (trifluoromethyl)cyclo- propyl]methoxy]-3- pyridyl]methanone | A.1 and B.3 | 462.3 [M + H]⁺ |
| 28 | 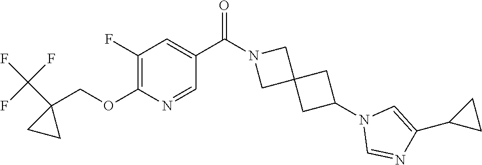 [SMILES: O=C(c1cnc(OCC2(C(F)(F)F)CC2)c(F)c1)N1CC2(CC(n3cnc(C4CC4)c3)C2)C1] | [6-(4- cyclopropylimidazol-1- yl)-2- azaspiro[3.3]heptan-2- yl]-[5-fluoro-6-[[1- (trifluoromethyl)cyclo- propyl]methoxy]-3- pyridyl]methanone | A.2 and B.4 | 465.2 [M + H]⁺ |
| 25 | 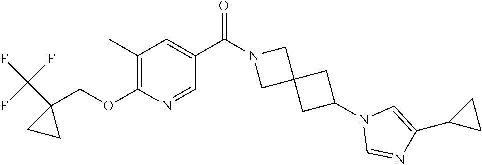 [SMILES: Cc1cc(C(=O)N2CC3(CC(n4cnc(C5CC5)c4)C3)C2)cnc1OCC1(C(F)(F)F)CC1] | [6-(4- cyclopropylimidazol-1- yl)-2- azaspiro[3.3]heptan-2- yl]-[5-methyl-6-[[1- (trifluoromethyl)cyclo- propyl]methoxy]-3- pyridyl]methanone | A.2 and B.3 | 461.2 [M + H]⁺ |
| 27 | 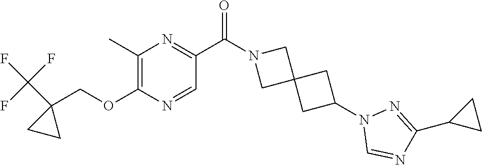 [SMILES: Cc1nc(C(=O)N2CC3(CC(n4cnc(C5CC5)n4)C3)C2)cnc1OCC1(C(F)(F)F)CC1] | [6-(3-cyclopropyl- 1,2,4-triazol-1-yl)-2- azaspiro[3.3]heptan-2- yl]-[6-methyl-5-[[1- (trifluoromethyl)cyclo- propyl]methoxy] pyrazin-2-yl]methanone | A.1 and B.5 | 463.3 [M + H]⁺ |
| 31 | 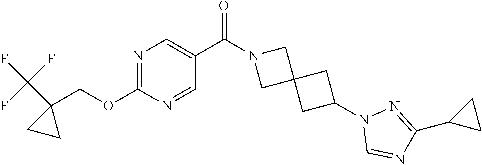 [SMILES: O=C(c1cnc(OCC2(C(F)(F)F)CC2)nc1)N1CC2(CC(n3cnc(C4CC4)n3)C2)C1] | [6-(3-cyclopropyl- 1,2,4-triazol-1-yl)-2- azaspiro[3.3]heptan-2- yl]-[2-[[1- (trifluoromethyl)cyclo- propyl]methoxy] pyrimidin-5-yl] methanone | A.1 and B.6 | 449.3 [M + H]⁺ |
| 32 | 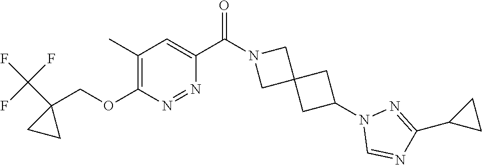 [SMILES: Cc1cc(C(=O)N2CC3(CC(n4cnc(C5CC5)n4)C3)C2)nnc1OCC1(C(F)(F)F)CC1] | [6-(3-cyclopropyl- 1,2,4-triazol-1-yl)-2- azaspiro[3.3]heptan-2- yl]-[5-methyl-6-[[1- (trifluoromethyl)cyclo- propyl]methoxy] pyridazin-3-yl] methanone | A.1 and B.7 | 463.3 [M + H]⁺ |
| 64 | 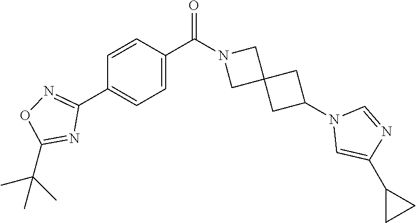 [SMILES: CC(C)(C)c1nc(-c2ccc(C(=O)N3CC4(CC(n5cnc(C6CC6)c5)C4)C3)cc2)no1] | (4-(5-(tert-butyl)-1,2,4- oxadiazol-3- yl)phenyl)(6-(4- cyclopropyl-1H- imidazol-1-yl)-2- azaspiro[3.3]heptan-2- yl)methanone | A.2 and CAS: 1119452- 72-0 | 432.4 [M + H]⁺ |
| 159 | 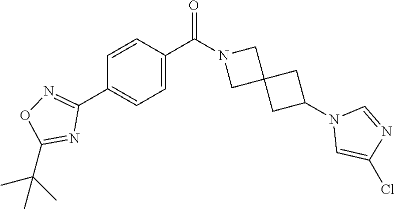 [SMILES: CC(C)(C)c1nc(-c2ccc(C(=O)N3CC4(CC(n5cnc(Cl)c5)C4)C3)cc2)no1] | [4-(5-tert-butyl-1,2,4- oxadiazol-3-yl)phenyl]- [6-(4-chloroimidazol-1- yl)-2- azaspiro[3.3]heptan-2- yl]methanone | A.7 and CAS: 1119452- 72-0 | 426.2 [M + H]⁺ |
| 162 | 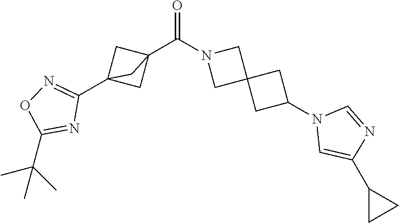 [SMILES: CC(C)(C)c1nc(C23CC(C(=O)N4CC5(CC(n6cnc(C7CC7)c6)C5)C4)(C2)C3)no1] | [3-(5-tert-butyl-1,2,4- oxadiazol-3-yl)-1- bicyclo[1.1.1]pentanyl]- [6-(4- cyclopropylimidazol-1- yl)-2- azaspiro[3.3]heptan-2- yl]methanone | A.2 and B.57 | 422.5 [M + H]⁺ |
| 163 | 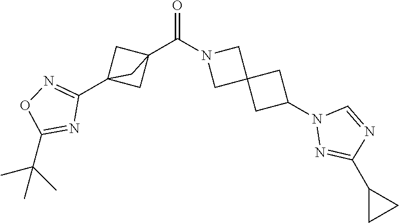 [SMILES: CC(C)(C)c1nc(C23CC(C(=O)N4CC5(CC(n6cnc(C7CC7)n6)C5)C4)(C2)C3)no1] | [3-(5-tert-butyl-1,2,4- oxadiazol-3-yl)-1- bicyclo[1.1.1]pentanyl]- [6-(3-cyclopropyl- 1,2,4-triazol-1-yl)-2- azaspiro[3.3]heptan-2- yl]methanone | A.1 and B.57 | 423.4 [M + H]⁺ |
| 168 | 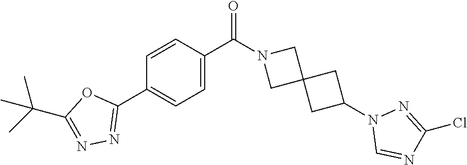 [SMILES: CC(C)(C)c1nnc(-c2ccc(C(=O)N3CC4(CC(n5cnc(Cl)n5)C4)C3)cc2)o1] | [4-(5-tert-butyl-1,3,4- oxadiazol-2-yl)phenyl]- [6-(3-chloro-1,2,4- triazol-1-yl)-2- azaspiro[3.3]heptan-2- yl]methanone | A.8 and B.61 | 427.3 [M + H]⁺ |
| 169 | 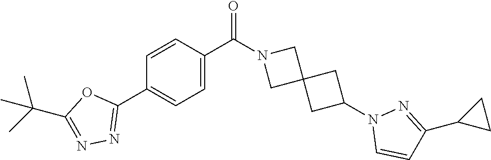 [SMILES: CC(C)(C)c1nnc(-c2ccc(C(=O)N3CC4(CC(n5ccc(C6CC6)n5)C4)C3)cc2)o1] | [4-(5-tert-butyl-1,3,4- oxadiazol-2-yl)phenyl]- [6-(3- cyclopropylpyrazol-1- yl)-2- azaspiro[3.3]heptan-2- yl]methanone | A.6 and B.61 | 432.3 [M + H]⁺ |
| 170 | 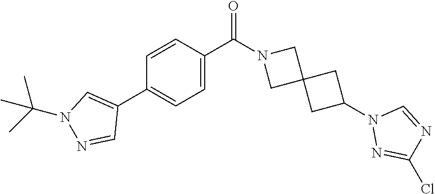 [SMILES: CC(C)(C)n1cc(-c2ccc(C(=O)N3CC4(CC(n5cnc(Cl)n5)C4)C3)cc2)cn1] | [4-(1-tert-butylpyrazol- 4-yl)phenyl]-[6-(3- chloro-1,2,4-triazol-1- yl)-2- azaspiro[3.3]heptan-2- yl]methanone | A.8 and B.62 | 425.2 [M + H]⁺ |
| 171 | 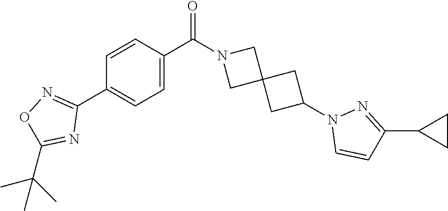 [SMILES: CC(C)(C)c1nc(-c2ccc(C(=O)N3CC4(CC(n5ccc(C6CC6)n5)C4)C3)cc2)no1] | [4-(5-tert-butyl-1,2,4- oxadiazol-3-yl)phenyl]- [6-(3- cyclopropylpyrazol-1- yl)-2- azaspiro[3.3]heptan-2- yl]methanone | A.6 and CAS: 1119452- 72-0 | 432.4 [M + H]⁺ |
| 172 | 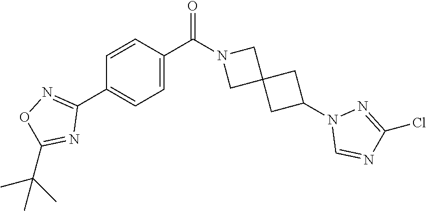 [SMILES: CC(C)(C)c1nc(-c2ccc(C(=O)N3CC4(CC(n5cnc(Cl)n5)C4)C3)cc2)no1] | [4-(5-tert-butyl-1,2,4- oxadiazol-3-yl)phenyl]- [6-(3-chloro-1,2,4- triazol-1-yl)-2- azaspiro[3.3]heptan-2- yl]methanone | A.8 and CAS: 1119452- 72-0 | 427.2 [M + H]⁺ |
| 180 | 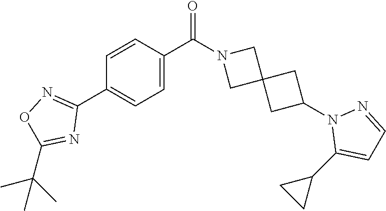 [SMILES: CC(C)(C)c1nc(-c2ccc(C(=O)N3CC4(CC(n5nccc5C5CC5)C4)C3)cc2)no1] | [4-(5-tert-butyl-1,2,4- oxadiazol-3-yl)phenyl]- [6-(5- cyclopropylpyrazol-1- yl)-2- azaspiro[3.3]heptan-2- yl]methanone | A.3 and CAS: 1119452- 72-0 | 432.4 [M + H]⁺ |

### Example 33

### [6-(3-cyclopropyl-1,2,4-triazol-1-yl)-2-azaspiro[3.3]heptan-2-yl]−[3-[4-(trifluoromethoxy)phenyl]azetidin-1-yl]methanone

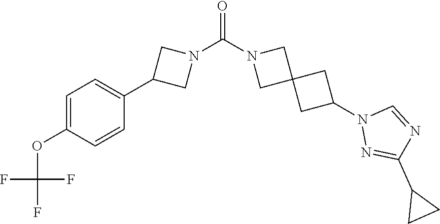 [SMILES: O=C(N1CC(c2ccc(OC(F)(F)F)cc2)C1)N1CC2(CC(n3cnc(C4CC4)n3)C2)C1]

To a solution of [6-(3-cyclopropyl-1,2,4-triazol-1-yl)-2-azaspiro[3.3]heptan-2-yl]-(3-iodoazetidin-1-yl)methanone (92.3 mg, 0.223 mmol) and (4-(trifluoromethoxy)phenyl)boronic acid (69.1 mg) in i-PrOH (3.00 mL) was added NiI₂ (34.8 mg), trans-2-aminocyclohexanol hydrochloride (16.9 mg) and NaHMDS (0.446 mL, 1 M). The mixture was stirred at 80° C. for 16 h. The reaction mixture was concentrated by speedvac. The residue was purified by prep. HPLC (FA as modifer) to give final compound. MS (ESI): m/z=448.3 [M+H]⁺.

### Step a) 3-iodoazetidine

To a solution of tert-butyl 3-iodoazetidine-1-carboxylate (10 g, 35.3 mmol) in 2,2,2-trifluoroethanol (30 mL) was added ethoxyethane trifluoroborane; hydrofluoride (14.9 g, 45.9 mmol, 12.60 mL, 50-55% purity, 1.3 eq.). The mixture was stirred at 25° C. for 16 h. The solvent was removed under reduced pressure and 50 mL of DCM was added. The mixture was stirred at 25° C. for 30 min. Then the mixture was filtered to give white solid. The crude product 3-iodoazetidine (7.78 g, crude, HBF₄ salt) as a light yellow solid was used into the next step without further purification. MS (ESI): m/z=184.1 [M+H]⁺.

### Step b) [6-(3-cyclopropyl-1,2,4-triazol-1-yl)-2-azaspiro[3.3]heptan-2-yl]-(3-iodoazetidin-1-yl)methanone

To a solution of 3-iodoazetidine (2.69 g, 9.95 mmol, HBF₄ salt) in DCM (80 mL) was added DIEA (5.14 g, 39.8 mmol, 6.93 mL) and triphosgene (974 mg, 3.28 mmol) at 0° C. The mixture was stirred at 0° C. for 30 min then 6-(3-cyclopropyl-1,2,4-triazol-1-yl)-2-azaspiro[3.3]heptane (A.1) (3.78 g, 9.95 mmol, 1 eq., 2 HBF₄ salt) and DIEA (2.57 g, 19.9 mmol, 3.47 mL) was added. Then the mixture was stirred at 25° C. for 2 h. The mixture was washed with 40 mL of H₂O and extracted with DCM (60 mL×2). The organic layers were combined, dried over anhydrous Na₂SO₄ and concentrated to give crude product. The residue was purified by flash silica gel chromatography, eluting with 0-70% THF/Petroleum ether gradient to give the title compound (1.54 g, 2.86 mmol, 28.7% yield) as a white solid. MS (ESI): m/z=414.0 [M+H]⁺.

In analogy to Example 33, the Examples in the following table were generated, using the commercial boronic acid building blocks.

| | | | | MS, |
|---|---|---|---|---|
| | | | Building | ESI: |
| Ex. | Structure | Systematic Name | Blocks | m/z |
| 34 | 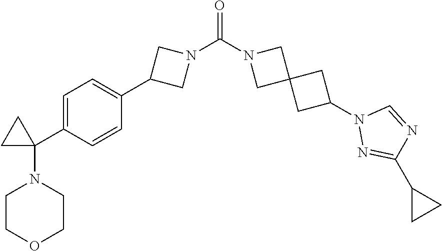 [SMILES: O=C(N1CC(c2ccc(C3(N4CCOCC4)CC3)cc2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[4-(1- morpholinocyclopro- pyl)phenyl]azetidin-1- yl]methanone | 4-(1-(4- (4,4,5,5- tetramethyl- 1,3,2- dioxaborolan- 2-yl)phenyl) cyclopropyl) morpholine (CAS: 1206594-08-2) | 489.3 [M + H]⁺ |
| 36 | 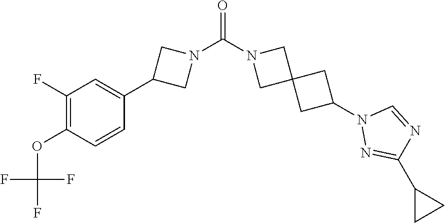 [SMILES: O=C(N1CC(c2ccc(OC(F)(F)F)c(F)c2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[3-fluoro-4- (trifluoromethoxy) phenyl]azetidin-1- yl]methanone | (3-fluoro-4- (trifluoro- methoxy) phenyl) boronic acid (CAS: 187804- 79-1) | 466.2 [M + H]⁺ |
| 37 | 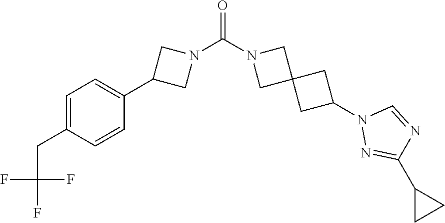 [SMILES: O=C(N1CC(c2ccc(CC(F)(F)F)cc2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[4-(2,2,2- trifluoroethyl)phenyl] azetidin-1- yl]methanone | 4,4,5,5- tetramethyl-2- [4-(2,2,2- trifluoroethyl) phenyl]-1,3,2- dioxaborolane (CAS: 1310949-87-1) | 446.3 [M + H]⁺ |
| 38 | 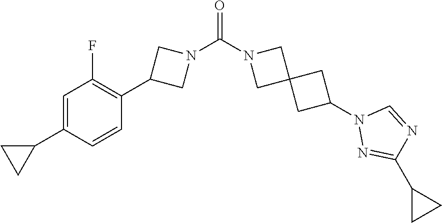 [SMILES: O=C(N1CC(c2ccc(C3CC3)cc2F)C1)N1CC2(CC(n3cnc(C4CC4)n3)C2)C1] | [3-(4-cyclopropyl-2- fluoro- phenyl)azetidin-1-yl]- [6-(3-cyclopropyl- 1,2,4-triazol-1-yl)-2- azaspiro[3.3]heptan-2- yl]methanone | 2-(4- cyclopropyl-2- fluorophenyl)- 4,4,5,5- tetramethyl- 1,3,2- dioxaborolane (CAS: 1338718-13-0) | 422.3 [M + H]⁺ |

### Example 35 [6-(3-cyclopropyl-1,2,4-triazol-1-yl)-2-azaspiro13.31heptan-2-yl]-13-[4-(1,1-difluoroethyl)phenyl]azetidin-1-yl]methanone

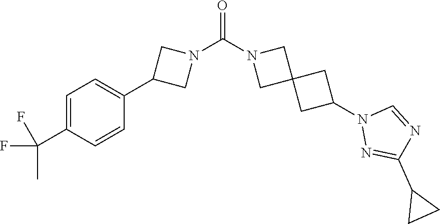 [SMILES: CC(F)(F)c1ccc(C2CN(C(=O)N3CC4(CC(n5cnc(C6CC6)n5)C4)C3)C2)cc1]

To a solution of (6-(3-cyclopropyl-1H-1,2,4-triazol-1-yl)-2-azaspiro[3.3]heptan-2-yl)(3-iodoazetidin-1-yl)methanone (Example 33, Step b) (92.3 mg, 0.223 mmol) and (4-(1,1-difluoroethyl) phenyl) boronic acid (62.3 mg, 0.335 mmol) in DMF (3.0 mL) was added Cs₂CO₃ aq. (2.0 M, 335 μl, 0.669 mmol), CyJohn Phos (3.86 mg, 0.011 mmol) and Pd₂(dba)₃ (10.2 mg, 0.011 mmol). The mixture was stirred at 80° C. for 16 h. The reaction mixture was concentrated by Speedvac. The residue was purified by preparative HPLC, and then further purified by preparative TLC to give the title compound (13.4 mg). MS (ESI): m/z=428.2 [M+H]⁺.

### Example 84

### [3-[2-chloro-4-(trifluoromethoxy)phenyl]azetidin-1-yl]−[6-(3-cyclopropyl-1,2,4-triazol-1-yl)-2-azaspiro[3.3]heptan-2-yl]methanone

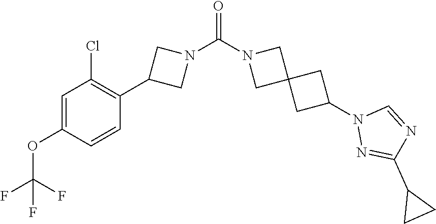 [SMILES: O=C(N1CC(c2ccc(OC(F)(F)F)cc2Cl)C1)N1CC2(CC(n3cnc(C4CC4)n3)C2)C1]

To a solution of 1-bromo-2-chloro-4-(trifluoromethoxy)benzene (CAS: 892845-59-9) (55.1 mg, 200 μmol) in DME (1.0 mL) was added (6-(3-cyclopropyl-1H-1,2,4-triazol-1-yl)-2-azaspiro[3.3]heptan-2-yl)(3-iodoazetidin-1-yl)methanone (Example 33, Step b) (41.3 mg, 100 μmol), Na₂CO₃ (42.4 mg, 400 μmol), Ir[dF(CF₃)ppy]₂(dtbbpy)PF₆ (1.1 mg, 1 μmol), NiCl₂·DME (1.1 mg, 0.05 eq.), dtbbpy (1.3 mg, 5 μmol, 0.05 eq.) and (TMS)₃SiH (37.2 mg, 150 μmol) in glove box. The mixture was stirred at 25° C. for 16 h under 72 W blue LED strip. The mixture was filtered and washed with 1 mL of water. Then the mixture was extracted with EtOAc (2 mL×2). The organic layer was dried over anhydrous Na₂SO₄ and concentrated to give crude product. The crude product was purified by preparative HPLC to the title compound (16.4 mg). MS (ESI): m/z=482.2 [M+H]⁺.

In analogy to Example 84, the Examples in the following table were generated, using the commercial bromide building blocks.

| | | | | MS, |
|---|---|---|---|---|
| | | | Building | ESI: |
| Ex. | Structure | Systematic Name | Blocks | m/z |
| 39 | 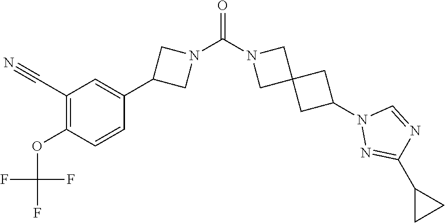 [SMILES: N#Cc1cc(C2CN(C(=O)N3CC4(CC(n5cnc(C6CC6)n5)C4)C3)C2)ccc1OC(F)(F)F] | 5-[1-[6-(3- cyclopropyl-1,2,4- triazol-1-yl)-2- azaspiro[3.3]heptane- 2-carbonyl]azetidin-3- yl]-2- (trifluoromethoxy) benzonitrile | 5-bromo-2- (trifluoro- methoxy) benzonitrile (CAS: 1210906-15-2) | 473.2 [M + H]⁺ |
| 85 | 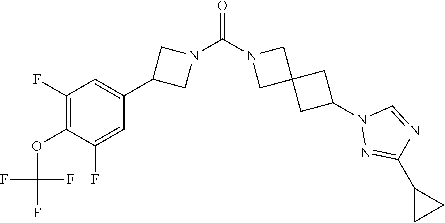 [SMILES: O=C(N1CC(c2cc(F)c(OC(F)(F)F)c(F)c2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[3,5-difluoro-4- (trifluoromethoxy) phenyl]azetidin-1- yl]methanone | 4-bromo-2,6- difluorotri- fluoro- methoxy- benzene (CAS: 115467-07-7) | 484.2 [M + H]⁺ |
| 86 | 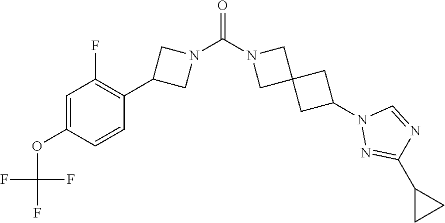 [SMILES: O=C(N1CC(c2ccc(OC(F)(F)F)cc2F)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[2-fluoro-4- (trifluoromethoxy) phenyl]azetidin-1- yl]methanone | 1-bromo-2- fluoro-4- (trifluoro- methoxy) benzene (CAS: 168971- 68-4) | 466.2 [M + H]⁺ |
| 87 | 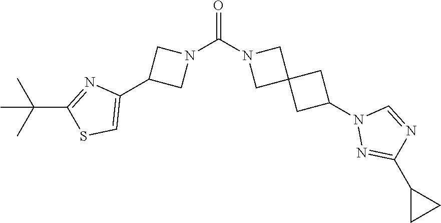 [SMILES: CC(C)(C)c1nc(C2CN(C(=O)N3CC4(CC(n5cnc(C6CC6)n5)C4)C3)C2)cs1] | [3-(2-tert-butylthiazol- 4-yl)azetidin-1-yl]-[6- (3-cyclopropyl-1,2,4- triazol-1-yl)-2- azaspiro[3.3]heptan-2- yl]methanone | 4-bromo-2- tert-butyl-1,3- thiazole (CAS: 959238-21-2) | 427.2 [M + H]⁺ |

### Example 40

### [6-(3-cyclopropyl-1,2,4-triazol-1-yl)-2-azaspiro[3.3]heptan-2-yl]−[6-[[2-methoxy-4-(trifluoromethyl)phenyl]methyl]-2-azaspiro[3.3]heptan-2-yl]methanone

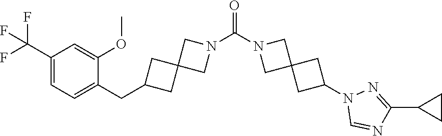 [SMILES: COc1cc(C(F)(F)F)ccc1CC1CC2(C1)CN(C(=O)N1CC3(CC(n4cnc(C5CC5)n4)C3)C1)C2]

To a solution of 6-(3-cyclopropyl-1H-1,2,4-triazol-1-yl)-2-azaspiro[3.3]heptane 4-methylbenzenesulfonate (A.1; salt) (40 mg, 106 μmol, Eq: 1) in acetonitrile (100 μL), triethylamine (75.3 mg, 104 μL, 744 μmol) and then di(1H-1,2,4-triazol-1-yl)methanone (17.4 mg, 106 μmol) were added. The mixture was stirred at room temperature for 3 h. 6-(2-methoxy-4-(trifluoromethyl)benzyl)-2-azaspiro[3.3]heptane 2,2,2-trifluoroacetate (B.8) (42.4 mg, 106 μmol) was added and the mixture was stirred at 70° C. for 16 h. The reaction mixture was diluted with water and extracted with ethyl acetate. The combined organic layers were dried over MgSO₄, filtered and concentrated in vacuo to give a crude residue which was submitted to rpHPLC for purification, to yield the title compound (11 mg, 20%) as a colourless amorphous solid. MS (ESI): m/z=515.3 [M+H]⁺.

In analogy to Example 40, Examples following table were generated, using the respective building blocks A.X and B.X. In some cases other salts of A.1 were used (e.g. trifluoroacetate), or alternative bases such as DIPEA.

| | | | | MS, |
|---|---|---|---|---|
| | | | Building | ESI: |
| Ex. | Structure | Systematic Name | Blocks | m/z |
| 44 | 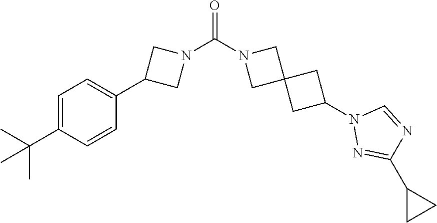 [SMILES: CC(C)(C)c1ccc(C2CN(C(=O)N3CC4(CC(n5cnc(C6CC6)n5)C4)C3)C2)cc1] | [3-(4-tert- butylphenyl)azetidin-1- yl]-[6-(3-cyclopropyl- 1,2,4-triazol-1-yl)-2- azaspiro[3.3]heptan-2- yl]methanone | A.1 and B.11 | 420.3 [M + H]⁺ |
| 48 | 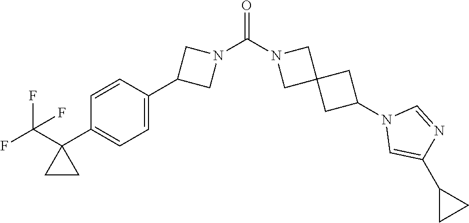 [SMILES: O=C(N1CC(c2ccc(C3(C(F)(F)F)CC3)cc2)C1)N1CC2(CC(n3cnc(C4CC4)c3)C2)C1] | [6-(4- cyclopropylimidazol-1- yl)-2- azaspiro[3.3]heptan-2- yl]-[3-[4-[1- (trifluoromethyl)cyclo- propyl]phenyl]azetidin- 1-yl]methanone | A.2 and B.1 | 471.5 [M + H]⁺ |
| 49 | 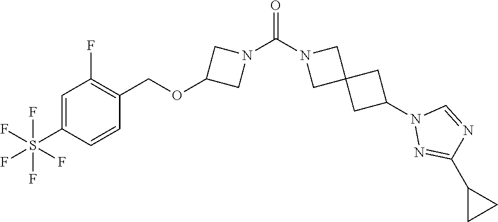 [SMILES: O=C(N1CC(OCc2ccc(S(F)(F)(F)(F)F)cc2F)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[[2-fluoro-4- (pentafluoro-26- sulfanyl)phenyl] methoxyazetidin-1- yl]methanone | A.1 and B.44 | 538.3 [M + H]⁺ |
| 69 | 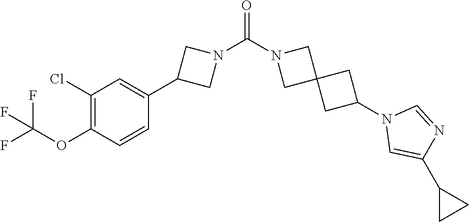 [SMILES: O=C(N1CC(c2ccc(OC(F)(F)F)c(Cl)c2)C1)N1CC2(CC(n3cnc(C4CC4)c3)C2)C1] | [3-[3-chloro-4- (trifluoromethoxy) phenylazetidin- 1-yl]-[6-(4- cyclopropylimidazol-1- yl)-2- azaspiro[3.3]heptan-2- yl]methanone | A.2 and B.15 | 481.3 [M + H]⁺ |
| 89 | 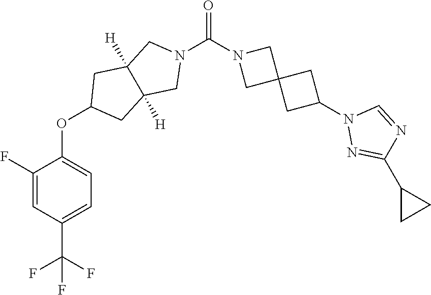 [SMILES: O=C(N1C[C@H]2CC(Oc3ccc(C(F)(F)F)cc3F)C[C@H]2C1)N1CC2(CC(n3cnc(C4CC4)n3)C2)C1] | [6-(3-cyclopropyl- 1,2,4-triazol-1-yl)-2- azaspiro[3.3]heptan-2- yl]-[rac-(3aS,6aR)-5-[2- fluoro-4- (trifluoromethyl) phenoxy]-3,3a,4,5,6,6a- hexahydro-1H- cyclopenta[c]pyrrol-2- yl]methanone | A.1 and B.33 | 520.4 [M + H]⁺ |
| 91 | 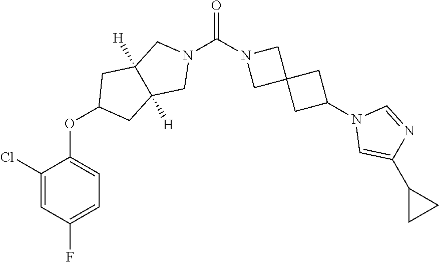 [SMILES: O=C(N1C[C@H]2CC(Oc3ccc(F)cc3Cl)C[C@H]2C1)N1CC2(CC(n3cnc(C4CC4)c3)C2)C1] | [6-(4- cyclopropylimidazol-1- yl)-2- azaspiro[3.3]heptan-2- yl]-[rac-(3aR,6aS)-5-(2- chloro-4-fluoro- phenoxy)-3,3a,4,5,6,6a- hexahydro-1H- cyclopenta[c]pyrrol-2- yl]methanone | A.2 and B.34 | 485.4 [M + H]⁺ |
| 92 | 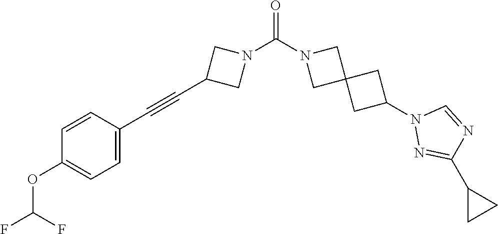 [SMILES: O=C(N1CC(C#Cc2ccc(OC(F)F)cc2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[2-[4- (difluoromethoxy) phenyl]ethynyl] azetidin-1- yl]methanone | A.1 and B.35 | 454.3 [M + H]⁺ |
| 93 | 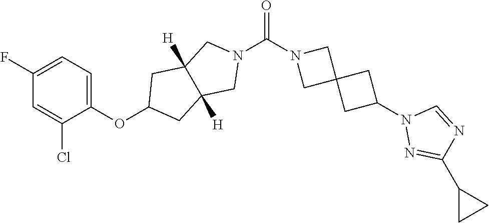 [SMILES: O=C(N1C[C@H]2CC(Oc3ccc(F)cc3Cl)C[C@H]2C1)N1CC2(CC(n3cnc(C4CC4)n3)C2)C1] | [6-(3-cyclopropyl- 1,2,4-triazol-1-yl)-2- azaspiro[3.3]heptan-2- yl]-[rac-(3aR,6aS)-5-(2- chloro-4-fluoro- phenoxy)-3,3a,4,5,6,6a- hexahydro-1H- cyclopenta[c]pyrrol-2- yl]methanone | A.1 and B.34 | 486.3 [M + H]⁺ |
| 101 | 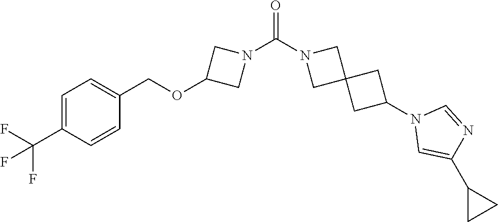 [SMILES: O=C(N1CC(OCc2ccc(C(F)(F)F)cc2)C1)N1CC2(CC(n3cnc(C4CC4)c3)C2)C1] | [6-(4- cyclopropylimidazol-1- yl)-2- azaspiro[3.3]heptan-2- yl]-[3-[[4- (trifluoromethyl)phenyl] methoxy]azetidin-1- yl]methanone | A.2 and B.20 | 461.4 [M + H]⁺ |
| 102 | 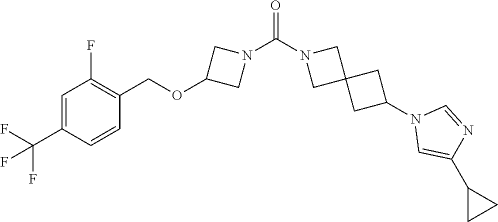 [SMILES: O=C(N1CC(OCc2ccc(C(F)(F)F)cc2F)C1)N1CC2(CC(n3cnc(C4CC4)c3)C2)C1] | [6-(4- cyclopropylimidazol-1- yl)-2- azaspiro[3.3]heptan-2- yl]-[3-[[2-fluoro-4- (trifluoromethyl)phenyl] methoxy]azetidin-1- yl]methanone | A.2 and B.21 | 479.3 [M + H]⁺ |
| 103 | 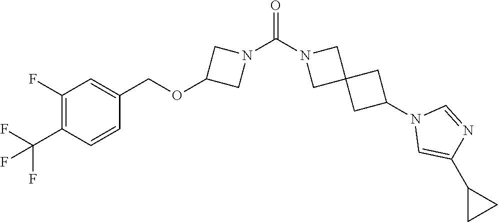 [SMILES: O=C(N1CC(OCc2ccc(C(F)(F)F)c(F)c2)C1)N1CC2(CC(n3cnc(C4CC4)c3)C2)C1] | [6-(4- cyclopropylimidazol-1- yl)-2- azaspiro[3.3]heptan-2- yl]-[3-[[3-fluoro-4- (trifluoromethyl)phenyl] methoxy]azetidin-1- yl]methanone | A.2 and B.22 | 479.3 [M + H]⁺ |
| 104 | 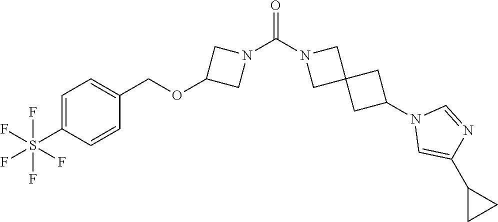 [SMILES: O=C(N1CC(OCc2ccc(S(F)(F)(F)(F)F)cc2)C1)N1CC2(CC(n3cnc(C4CC4)c3)C2)C1] | [6-(4- cyclopropylimidazol-1- yl)-2- azaspiro[3.3]heptan-2- yl]-[3-[[4-(pentafluoro- λ⁶- sulfanyl)phenyl] methoxy]azetidin-1- yl]methanone | A.2 and B.43 | 519.3 [M + H]⁺ |
| 105 | 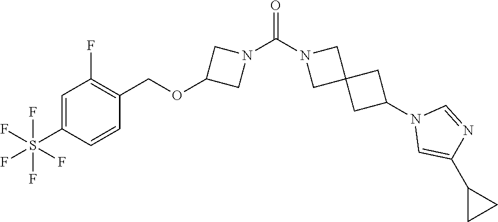 [SMILES: O=C(N1CC(OCc2ccc(S(F)(F)(F)(F)F)cc2F)C1)N1CC2(CC(n3cnc(C4CC4)c3)C2)C1] | [6-(4- cyclopropylimidazol-1- yl)-2- azaspiro[3.3]heptan-2- yl]-[3-[[2-fluoro-4- (pentafluoro-λ⁶- sulfanyl)phenyl] methoxy]azetidin-1- yl]methanone | A.2 and B.44 | 537.2 [M + H]⁺ |
| 106 | 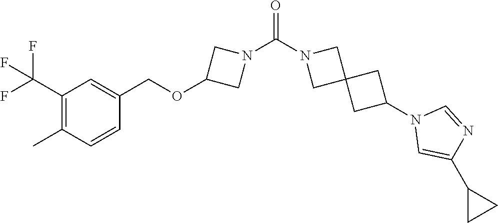 [SMILES: Cc1ccc(COC2CN(C(=O)N3CC4(CC(n5cnc(C6CC6)c5)C4)C3)C2)cc1C(F)(F)F] | [6-(4- cyclopropylimidazol-1- yl)-2- azaspiro[3.3]heptan-2- yl]-[3-[[4-methyl-3- (trifluoromethyl)phenyl] methoxy]azetidin-1- yl]methanone | A.2 and B.45 | 475.3 [M + H]⁺ |
| 107 | 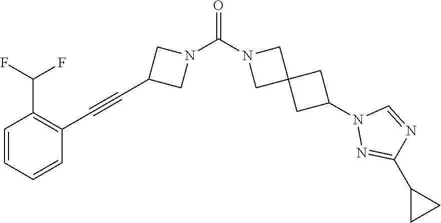 [SMILES: O=C(N1CC(C#Cc2ccccc2C(F)F)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[2-[2- (difluoromethyl)phenyl] ethynyl]azetidin-1- yl]methanone | A.1 and B.46 | 438.2 [M + H]⁺ |
| 108 | 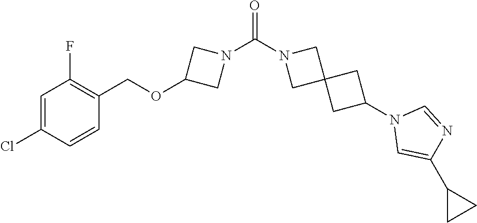 [SMILES: O=C(N1CC(OCc2ccc(Cl)cc2F)C1)N1CC2(CC(n3cnc(C4CC4)c3)C2)C1] | [3-[(4-chloro-2-fluoro- phenyl)methoxy] azetidin-1-yl]-[6-(4- cyclopropylimidazol-1- yl)-2- azaspiro[3.3]heptan-2- yl]methanone | A.2 and B.47 | 445.3 [M + H]⁺ |
| 109 | 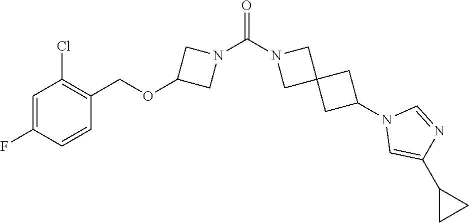 [SMILES: O=C(N1CC(OCc2ccc(F)cc2Cl)C1)N1CC2(CC(n3cnc(C4CC4)c3)C2)C1] | [3-[(2-chloro-4-fluoro- phenyl)methoxy] azetidin-1-yl]-[6-(4- cyclopropylimidazol-1- yl)-2- azaspiro[3.3]heptan-2- yl]methanone | A.2 and B.25 | 445.3 [M + H]⁺ |
| 110 | 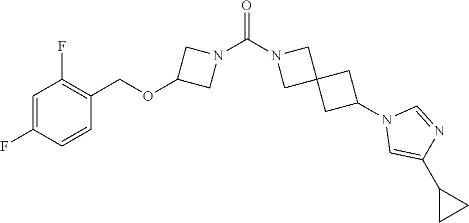 [SMILES: O=C(N1CC(OCc2ccc(F)cc2F)C1)N1CC2(CC(n3cnc(C4CC4)c3)C2)C1] | [6-(4- cyclopropylimidazol-1- yl)-2- azaspiro[3.3]heptan-2- yl]-[3-[(2,4- difluorophenyl) methoxy]azetidin-1- yl]methanone | A.2 and B.48 | 429.3 [M + H]⁺ |

### Example 42

### [6-(3-cyclopropyl-1,2,4-triazol-1-yl)-2-azaspiro[3.3]heptan-2-yl]−[6-[2-fluoro-4-(trifluoromethoxy)phenoxy]-2-azaspiro[3.3]heptan-2-yl]methanone

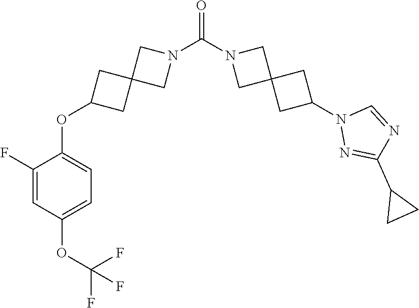 [SMILES: O=C(N1CC2(CC(Oc3ccc(OC(F)(F)F)cc3F)C2)C1)N1CC2(CC(n3cnc(C4CC4)n3)C2)C1]

To a solution of 2-fluoro-4-(trifluoromethoxy)phenol (39.1 mg, 199 μmol) in dry DMF (1 mL) was added NaH (7.97 mg, 199 μmol) and the reaction mixture was stirred at r.t for 10 min followed by addition of 2-(6-(3-cyclopropyl-1H-1,2,4-triazol-1-yl)-2-azaspiro[3.3]heptane-2-carbonyl)-2-azaspiro[3.3]heptan-6-yl methanesulfonate (70 mg, 166 μmol). The reaction mixture was stirred at 90° C. for 18 h. The crude reaction solution was directly submitted for reversed-phase HPLC purification to yield the title compound (74.3 mg, 84.1%). MS (ESI): m/z=522.3 [M+H]⁺.

### Step a) (6-(3-cyclopropyl-]H-1, 2,4-triazol-1-yl)-2-azaspiro[3.3]heptan-2-yl)(6-hydroxy-2-azaspiro[3.3]heptan-2-yl)methanone

To a solution of (6-(3-cyclopropyl-1H-1,2,4-triazol-1-yl)-2-azaspiro[3.3]heptan-2-yl)(1H-1,2,4-triazol-1-yl)methanone (Example 1, Step a) (350 mg, 1.17 mmol) in dry DMF (7 mL) under an inert atmosphere was added 2-azaspiro[3.3]heptan-6-ol (159 mg, 1.4 mmol) and DIPEA (378 mg, 511 μL, 2.92 mmol). The reaction mixture was then stirred at 80° C. for 20 h. A further addition of 2-azaspiro[3.3]heptan-6-ol (79.4 mg, 702 μmol) and DIPEA (151 mg, 204 μL, 1.17 mmol) after which the reaction was again stirred at 80° C. for 6 h. Volatiles were removed in vacuo and the crude residue was purified by flash chromatography with an eluant mixture of dichloromethane and methanol (2% to 15%) to yield the impure title compound (393 mg), which was again purified by flash chromatography (eluent mixture of heptane and a solution of EtOAc:EtOH 3:1 (60% to 100%)) to yield the title compound (301 mg, 73.5%). MS (ESI): m/z=344.3 [M+H]⁺.

### Step b) 2-(6-(3-cyclopropyl-]H-1,2,4-triazol-1-yl)-2-azaspiro[3.3]heptane-2-carbonyl)-2-azaspiro[3.3]heptan-6-yl methanesulfonate

To a solution of (6-(3-cyclopropyl-1H-1,2,4-triazol-1-yl)-2-azaspiro[3.3]heptan-2-yl)(6-hydroxy-2-azaspiro[3.3]heptan-2-yl)methanone (300 mg, 874 μmol) in dry CH₂Cl₂ (8 mL) was added triethylamine (177 mg, 244 μL, 1.75 mmol) and methanesulfonyl chloride (120 mg, 81.4 μL, 1.05 mmol). The reaction mixture was stirred at r.t for 18 h. The reaction mixture was diluted with dichloromethane and extracted with water, the organic phase was collected and the aqueous phase was back-extracted with dichloromethane. The combined organic phases were dried over sodium sulfate and evaporated down to dryness to yield the crude title compound (360 mg), which was used without further purification. MS (ESI): m/z=422.4 [M+H]⁺.

In analogy to Example 42, the Examples in the following table were generated, using the commercial phenol/alcohol building blocks.

| | | | | MS, |
|---|---|---|---|---|
| | | | Building | ESI: |
| Ex. | Structure | Systematic Name | Blocks | m/z |
| 47 | 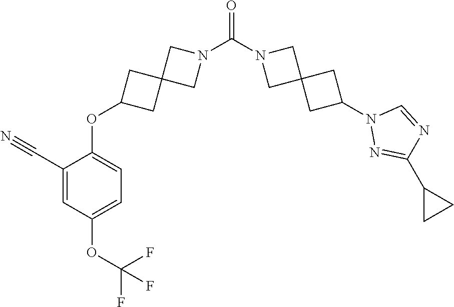 [SMILES: N#Cc1cc(OC(F)(F)F)ccc1OC1CC2(C1)CN(C(=O)N1CC3(CC(n4cnc(C5CC5)n4)C3)C1)C2] | 2-[[2-[6-(3- cyclopropyl-1,2,4- triazol-1-yl)-2- azaspiro[3.3]heptane- 2-carbonyl]-2- azaspiro[3.3]heptan-6- yloxy]-5- (trifluoromethoxy) benzonitrile | 2-hydroxy-5- (trifluoro- methoxy) benzonitrile (CAS: 875664- 40-7) | 529.3 [M + H]⁺ |
| 56 | 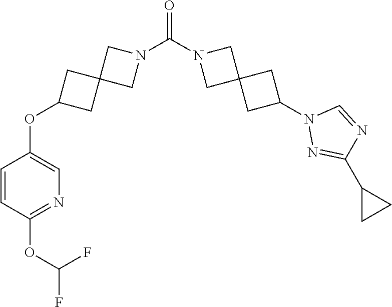 [SMILES: O=C(N1CC2(CC(Oc3ccc(OC(F)F)nc3)C2)C1)N1CC2(CC(n3cnc(C4CC4)n3)C2)C1] | (6-(3-cyclopropyl-1H- 1,2,4-triazol-1-yl)-2- azaspiro[3.3]heptan-2- yl)(6-((6- (difluoromethoxy) pyridin-3-yl)oxy)-2- azaspiro[3.3]heptan-2- yl)methanone | 6- (difluoro- methoxy) pyridin-3-ol (CAS: 859539- 04-1) | 487.3 [M + H]⁺ |
| 60 | 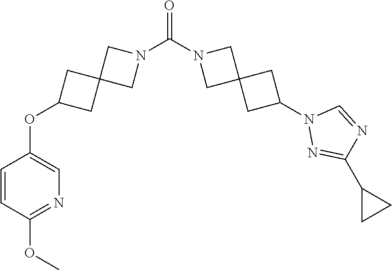 [SMILES: COc1ccc(OC2CC3(C2)CN(C(=O)N2CC4(CC(n5cnc(C6CC6)n5)C4)C2)C3)cn1] | (6-(3-cyclopropyl-1H- 1,2,4-triazol-1-yl)-2- azaspiro[3.3]heptan-2- yl)(6-((6- methoxypyridin-3- yl)oxy)-2- azaspiro[3.3]heptan-2- yl)methanone | 6- methoxy- pyridin- 3-ol (CAS: 51834-97-0) | 451.3 [M + H]⁺ |
| 63 | 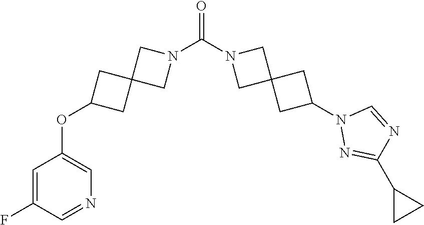 [SMILES: O=C(N1CC2(CC(Oc3cncc(F)c3)C2)C1)N1CC2(CC(n3cnc(C4CC4)n3)C2)C1] | (6-(3-cyclopropyl-1H- 1,2,4-triazol-1-yl)-2- azaspiro[3.3]heptan-2- yl)(6-((5- fluoropyridin-3- yl)oxy)-2- azaspiro[3.3]heptan-2- yl)methanone | 5- fluoropyridin- 3-ol | 439.3 [M + H]⁺ |
| 65 | 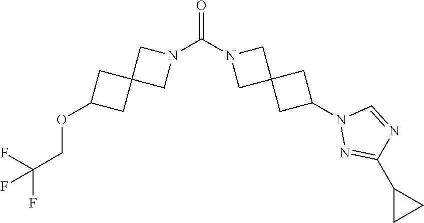 [SMILES: O=C(N1CC2(CC(OCC(F)(F)F)C2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[6-(2,2,2- trifluoroethoxy)-2- azaspiro[3.3]heptan-2- yl]methanone | 2,2,2- trifluoroethan- 1-ol | 426.3 [M + H]⁺ |
| 66 | 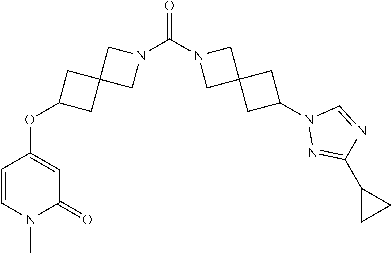 [SMILES: Cn1ccc(OC2CC3(C2)CN(C(=O)N2CC4(CC(n5cnc(C6CC6)n5)C4)C2)C3)cc1=O] | 4-[[2-[6-(3- cyclopropyl-1,2,4- triazol-1-yl)-2- azaspiro[3.3]heptane- 2-carbonyl]-2- azaspiro[3.3]heptan-6- yl]oxy]-1-methyl- pyridin-2-one | 4-hydroxy-1- methylpyridin- 2(1H)-one (CAS: 40357- 87-7) | 451.4 [M + H]⁺ |

### Example 55

### [6-(3-cyclopropyl-1,2,4-triazol-1-yl)-2-azaspiro[3.3]heptan-2-yl]−[6-[2-(trifluoromethyl)pyrimidin-4-yl]oxy-2-azaspiro[3.3]heptan-2-yl]methanone

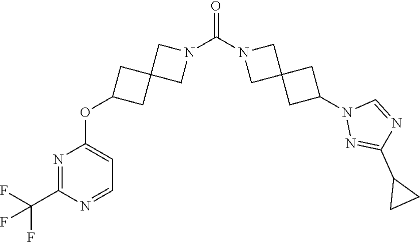 [SMILES: O=C(N1CC2(CC(Oc3ccnc(C(F)(F)F)n3)C2)C1)N1CC2(CC(n3cnc(C4CC4)n3)C2)C1]

To a solution of (6-(3-cyclopropyl-1H-1,2,4-triazol-1-yl)-2-azaspiro[3.3]heptan-2-yl)(6-hydroxy-2-azaspiro[3.3]heptan-2-yl)methanone (Example 42, Step a)) (60 mg, 175 μmol) in dry DMF (1 mL) under an inert atmosphere was added NaH (7.34 mg, 183 μmol) and the reaction mixture was stirred at r.t. for 10 min followed by addition of 4-chloro-2-(trifluoromethyl)pyrimidine (38.3 mg, 210 μmol). The reaction mixture was then stirred at 90° C. for 18 h. The crude reaction was directly submitted for reversed-phase HPLC purification to yield the title compound (36.4 mg). MS (ESI): m/z=490.3 [M+H]⁺.

In analogy to Example 55, the Examples in the following table were generated, using the commercial chloro-heteroaryl building blocks.

| | | | | MS, |
|---|---|---|---|---|
| | | | Building | ESI: |
| Ex. | Structure | Systematic Name | Blocks | m/z |
| 51 | 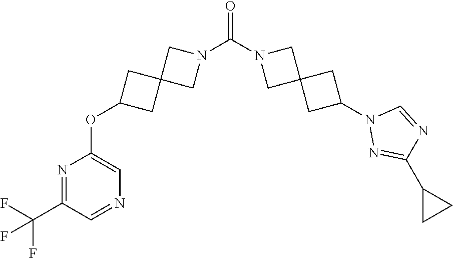 [SMILES: O=C(N1CC2(CC(Oc3cncc(C(F)(F)F)n3)C2)C1)N1CC2(CC(n3cnc(C4CC4)n3)C2)C1] | (6-(3-cyclopropyl-1H- 1,2,4-triazol-1-yl)-2- azaspiro[3.3]heptan-2- yl)(6-((6- (trifluoromethyl) pyrazin-2-yl)oxy)-2- azaspiro[3.3]heptan-2- yl)methanone | 2-chloro-6- (trifluoromethyl) pyrazine (CAS: 61655- 69-4) | 490.3 [M + H]⁺ |
| 52 | 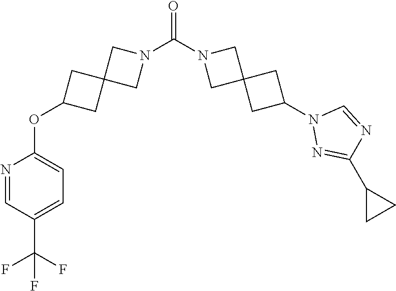 [SMILES: O=C(N1CC2(CC(Oc3ccc(C(F)(F)F)cn3)C2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[6-[[5- (trifluoromethyl)-2- pyridyl]oxy]-2- azaspiro[3.3]heptan-2- yl]methanone | 2-chloro-5- (trifluoromethyl) pyridine (CAS: 52334- 81-3) | 489.3 [M + H]⁺ |
| 57 | 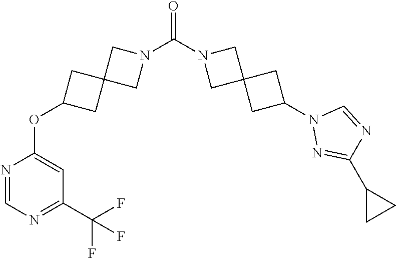 [SMILES: O=C(N1CC2(CC(Oc3cc(C(F)(F)F)ncn3)C2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[6-[6- (trifluoromethyl) pyrimidin-4-yl]oxy-2- azaspiro[3.3]heptan-2- yl]methanone | 4-chloro-6- (trifluoromethyl) pyrimidine (CAS: 37552- 81-1) | 490.3 [M + H]⁺ |
| 58 | 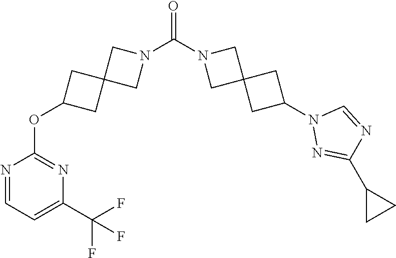 [SMILES: O=C(N1CC2(CC(Oc3nccc(C(F)(F)F)n3)C2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[6-[4- (trifluoromethyl) pyrimidin-2-yl]oxy-2- azaspiro[3.3]heptan-2- yl]methanone | 2-chloro-4- (trifluoromethyl) pyrimidine (CAS: 33034- 67-2) | 490.3 [M + H]⁺ |
| 61 | 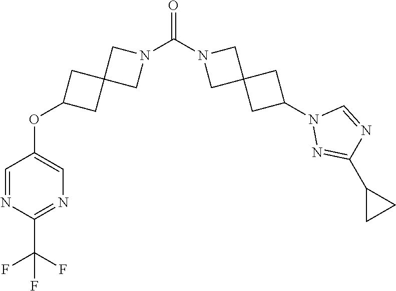 [SMILES: O=C(N1CC2(CC(Oc3cnc(C(F)(F)F)nc3)C2)C1)N1CC2(CC(n3cnc(C4CC4)n3)C2)C1] | (6-(3-cyclopropyl-1H- 1,2,4-triazol-1-yl)-2- azaspiro[3.3]heptan-2- yl)(6-((2- (trifluoromethyl) pyrimidin-5-yl)oxy)-2- azaspiro[3.3]heptan-2- yl)methanone | 5-bromo-2- (trifluoromethyl) pyrimidine (CAS: 436799- 32-5) | 490.3 [M + H]⁺ |
| 62 | 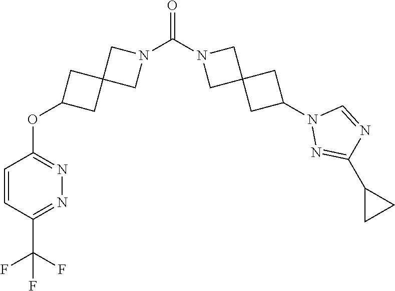 [SMILES: O=C(N1CC2(CC(Oc3ccc(C(F)(F)F)nn3)C2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[6-[6- (trifluoromethyl) pyridazin-3-yl]oxy-2- azaspiro[3.3]heptan-2- yl]methanone | 3-chloro-6- (trifluoromethyl) pyridazine | 490.3 [M + H]⁺ |
| 70 | 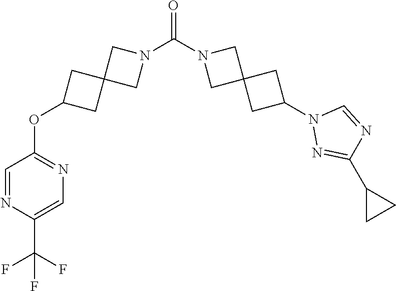 [SMILES: O=C(N1CC2(CC(Oc3cnc(C(F)(F)F)cn3)C2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[6-[5- (trifluoromethyl) pyrazin-2-yl]oxy-2- azaspiro[3.3]heptan-2- yl]methanone | 2-chloro-5- (trifluoromethyl) pyrazine | 490.3 [M + H]⁺ |

### Example 67

### [6-(5-cyclopropyl-1,2,4-triazol-1-yl)-2-azaspiro13.31heptan-2-yl]−[3-[4-[1-(trifluoromethyl)cyclopropyl]phenyl]azetidin-1-yl]₁methanone

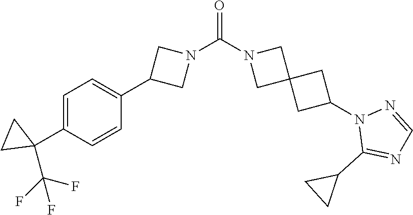 [SMILES: O=C(N1CC(c2ccc(C3(C(F)(F)F)CC3)cc2)C1)N1CC2(CC(n3ncnc3C3CC3)C2)C1]

To a solution of 4-nitrophenyl 6-(5-cyclopropyl-1H-1,2,4-triazol-1-yl)-2-azaspiro[3.3]heptane-2-carboxylate (25 mg, 67.7 μmol) in acetonitrile (1 mL) was added TEA (34.2 mg, 47.2 μL, 338 μmol) followed by 3-(4-(1-(trifluoromethyl)cyclopropyl)phenyl)azetidine 4-methylbenzenesulfonate (B.1) (42 mg, 102 μmol). The mixture was heated to 70° C. for 15 h. The volatiles were removed under reduced pressure. The crude was purified by column chromatography using DCM:methanol (0-10% methanol) as solvent. The title compound (12 mg, 24.2 μmol, 35.7% yield) was obtained as an off white solid. MS (ESI): m/z=472.4 [M+H]⁺.

### Step a) 4-nitrophenyl 6-(5-cyclopropyl-]H-1,2,4-triazol-1-yl)-2-azaspiro[3.3]heptane-2-carboxylate

To a solution of tert-butyl 6-(5-cyclopropyl-1,2,4-triazol-1-yl)-2-azaspiro[3.3]heptane-2-carboxylate (regioisomeric side product generated in Example A.1, Step b)) (457 mg, 1.5 mmol) in DCM (12.2 mL) was added 2,2,2-trifluoroacetic acid (1.71 g, 1.15 mL, 15 mmol). The mixture was stirred at room temperature for 5 hours. The solvent was removed and TFA was coevaporated with toluene. The crude was redissolved in dry DCM (12.2 mL). The mixture was cooled to 0° C. TEA (760 mg, 1.05 mL, 7.51 mmol) was added followed by 4-nitrophenyl carbonochloridate (303 mg, 1.5 mmol). The mixture was allowed to warm up to room temperature and stirred for an additional 12 h. The solvent was removed under reduced pressure. The crude was purified by column chromatography using heptane/ethyl acetate (1:1) as solvent. The title compound (195 mg, 528 μmol, 35.2% yield) was obtained as a light yellow solid. MS (ESI): m/z=370.1 [M+H]⁺.

### Example 88

### [6-(3-cyclopropyl-1,2,4-triazol-1-yl)-2-azaspiro[3.3]heptan-2-yl]−[4-[5-(2,2-dimethylpropyl)-1,2,4-oxadiazol-3-yl]phenyl]methanone

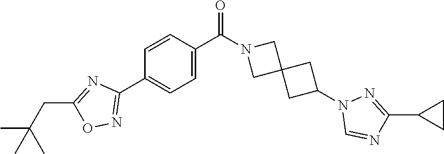 [SMILES: CC(C)(C)Cc1nc(-c2ccc(C(=O)N3CC4(CC(n5cnc(C6CC6)n5)C4)C3)cc2)no1]

To a suspension of 4-(5-neopentyl-1,2,4-oxadiazol-3-yl)benzoic acid (B.32) (44.6 mg, 171 μmol) in dry DMF (1 mL) was added DIPEA (90.6 mg, 122 μL, 701 μmol) and HATU (65.2 mg, 171 μmol). The reaction mixture was stirred at r.t. for 15 min followed by addition of 6-(3-cyclopropyl-1H-1,2,4-triazol-1-yl)-2-azaspiro[3.3]heptane bis(4-methylbenzenesulfonate) (A.1; tosylate salt) (90 mg, 156 μmol). The reaction mixture was stirred at r.t. for 60 min, cooled and directly submitted for reversed-phase HPLC purification to yield the title compound (41.6 mg). MS (ESI): m/z=447.4 [M+H]⁺.

### Example 90

### [4-(5-tert-butyl-1,2,4-oxadiazol-3-yl)phenyl]−[6-(3-cyclobutyl-1,2,4-triazol-1-yl)-2-azaspiro[3.3]heptan-2-yl]methanone

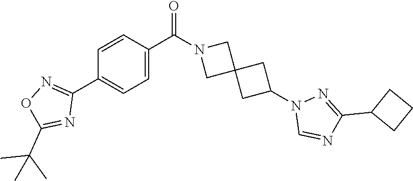 [SMILES: CC(C)(C)c1nc(-c2ccc(C(=O)N3CC4(CC(n5cnc(C6CCC6)n5)C4)C3)cc2)no1]

To a tert-butyl 6-(3-cyclobutyl-1H-1,2,4-triazol-1-yl)-2-azaspiro[3.3]heptane-2-carboxylate (165 mg, 518 μmol) in DCM (4.21 mL) was added TFA (591 mg, 399 μL, 5.18 mmol). The mixture was stirred at room temperature for 3 h. The solvent was removed under reduced pressure, TFA was coevaporated with toluene. The TFA salt was redissolved in DMF (4.21 mL) and DIPEA (402 mg, 543 μL, 3.11 mmol) was added followed by 4-(5-(tert-butyl)-1,2,4-oxadiazol-3-yl)benzoic acid (128 mg, 518 μmol) and 2,4,6-tripropyl-1,3,5,2,4,6-trioxatriphosphinane 2,4,6-trioxide (1.32 g, 942 μL, 2.07 mmol). The mixture was stirred at room temperature for 14 h. The mixture was diluted with ethyl acetate and washed with water. The aqeuous phase was extracted twice with ethyl acetate. The organic phase was washed with water and brine and dried over MgSO₄. The crude was purified by column chromatography using DCM/methanol (0-10% methanol) as solvent, followed by a further purification using column chromatography eluting with DCM/methanol (5% methanol). The title compound (79 mg, 163 μmol, 31.4% yield) was obtained as a white solid. MS (ESI): m/z=447.4 [M+H]⁺.

### Step a) tert-butyl 6-(3-cyclobutyl-]H-1, 2,4-triazol-1-yl)-2-azaspiro[3.3]heptane-2-carboxylate

To a solution of tert-butyl 6-((methylsulfonyl)oxy)-2-azaspiro[3.3]heptane-2-carboxylate (CAS: 1239320-11-6) (513 mg, 1.76 mmol) in DMF (13.5 mL) was added cesium carbonate (2.65 g, 8.12 mmol) followed by 3-cyclobutyl-1H-1,2,4-triazole (250 mg, 2.03 mmol). The mixture was stirred at 100° C. for 24 hours. The mixture was diluted with ethyl acetate and washed with water. The aqueous phase was extracted with ethyl acetate twice. The organic phase was washed with water and brine and dried over MgSO₄. The crude was purified by HPLC to yield the title compound as a white solid (170 mg, 26.3% yield). MS (ESI): m/z=319.3 [M+H]⁺.

### Example 112

### [6-(3-cyclopropyl-1,2,4-triazol-1-yl)-2-azaspiro[3.3]heptan-2-yl]−[rac-(3aS,6aR)-5-[4-(difluoromethoxy)-2-fluoro-phenoxy]-3,3a,4,5,6,6a-hexahydro-1H-cyclopenta[c]pyrrol-2-yl]methanone

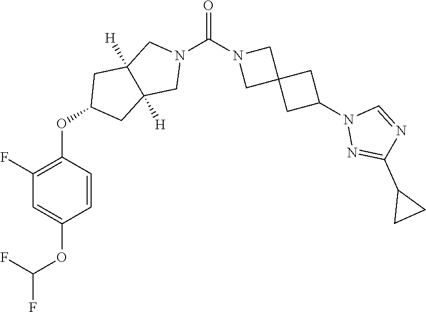 [SMILES: O=C(N1C[C@H]2C[C@H](Oc3ccc(OC(F)F)cc3F)C[C@H]2C1)N1CC2(CC(n3cnc(C4CC4)n3)C2)C1]

To a solution of (3aR,5s,6aS)-5-(4-(difluoromethoxy)-2-fluorophenoxy) octahydrocyclopenta [c]pyrrole (˜0.44 mmol, crude) in DCM (5 mL) was added TEA (2.64 mmol, 6.0 eq.). The mixture was cooled to 0° C. Triphosgene (0.145 mmol, 0.33 eq.) in DCM (1 mL) was added to the mixture. Then the mixture was stirred at 25° C. for 0.5 h. 6-(3-cyclopropyl-1,2,4-triazol-1-yl)-2-azaspiro[3.3]heptane (A.1) (0.44 mmol) was added. The mixture was stirred at 25° C. for 2 h. The mixture was quenched by H₂O (2 mL) and extracted with DCM (3 mL×2), dried over anhydrous Na₂SO₄ and concentrated to give crude product. The crude was purified by preparative HPLC to give (6-(3-cyclopropyl-1H-1,2,4-triazol-1-yl)-2-azaspiro[3.3]heptan-2-yl)((3aR,5s,6aS)-5-(4-(difluoromethoxy)-2-fluorophenoxy)hexahydrocyclopenta[c]pyrrol-2(1H)-yl)methanone (24.4 mg, 0.047 mmol, 10.7%). MS (ESI): m/z=518.2 [M+H]⁺.

### Step a) (3aR,5s,6aS)-tert-butyl 5-(4-(difluoromethoxy)-2-fluorophenoxy)hexahydrocyclopenta[c] pyrrole-2(H)-carboxylate

To a solution of 4-(difluoromethoxy)-2-fluorophenol (0.55 mmol, 1.25 eq.) in DCM (5 mL) was added (3aR,5r,6aS)-tert-butyl 5-hydroxyhexahydrocyclopenta[c]pyrrole-2(1H)-carboxylate (100 mg, 0.44 mmol, 1.0 eq.), PS-TPP (2.2 mmol, 5.0 eq., 3 mmol/g) and DIAD (0.88 mmol, 2.0 eq., 1.9 M in toluene) in glove box full of N₂. The mixture was shaken at 30° C. for 16 h and monitored by LCMS. The mixture was filtered and quenched with saturated aqueous NaHCO₃ solution (2.0 mL), extracted with DCM (3.0 mL×2) and dried over anhydrous Na₂SO₄. The solvent was removed under reduced pressure to give the title compound, which was used for next step without further purification. MS (ESI): m/z=332.2 [M+H-56]⁺.

### Step b) (3aR,5s, 6aS)-5-(4-(difluoromethoxy)-2-fluorophenoxy) octahydrocyclopenta [c]pyrrole

To a solution of(3aR,5s,6aS)-tert-butyl 5-(4-(difluoromethoxy)-2-fluorophenoxy) hexahydrocyclopenta[c]pyrrole-2(1H)-carboxylate (−0.44 mmol, crude, 1.0 eq.) in DCM (2 mL) was added 2 mL of HCl solution (8.0 mmol, 4.0 M in dioxane). The mixture was shaken at 30° C. for 2 h. The solvent was removed by Speedvac to give the crude title compound, which was used for next step without purification. MS (ESI): m/z=288.1 [M+H].

In analogy to Example 112, the Examples in the following table were generated, using the following commercial phenol building blocks in Step a).

| | | | | MS, |
|---|---|---|---|---|
| | | | Building | ESI: |
| Ex. | Structure | Systematic Name | Blocks | m/z |
| 113 | 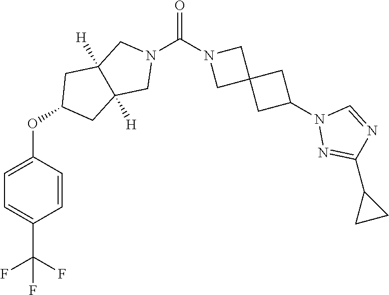 [SMILES: O=C(N1C[C@H]2C[C@H](Oc3ccc(C(F)(F)F)cc3)C[C@H]2C1)N1CC2(CC(n3cnc(C4CC4)n3)C2)C1] | [6-(3-cyclopropyl- 1,2,4-triazol-1-yl)-2- azaspiro[3.3]heptan-2- yl]-[rac-(3aS,6aR)-5- [4-(trifluoromethyl) phenoxy]-3,3a,4,5,6,6a- hexahydro-1H- cyclopenta[c]pyrrol-2- yl]methanone | 4- (trifluoromethyl) phenol (CAS: 402-45-9) | 502.2 [M + H]⁺ |
| 114 | 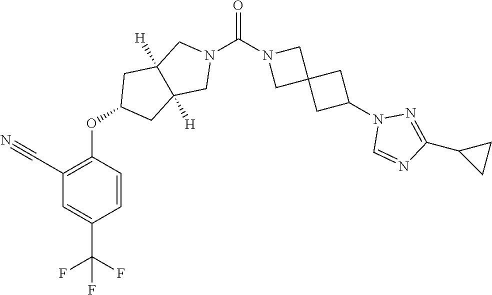 [SMILES: N#Cc1cc(C(F)(F)F)ccc1O[C@H]1C[C@@H]2CN(C(=O)N3CC4(CC(n5cnc(C6CC6)n5)C4)C3)C[C@@H]2C1] | 2-[[rac-(3aS,6aR)-2- [6-(3-cyclopropyl- 1,2,4-triazol-1-yl)-2- azaspiro[3.3]heptane- 2-carbonyl]- 3,3a,4,5,6,6a- hexahydro-1H- cyclopenta[c]pyrrol-5- yl]oxy]-5- (trifluoromethyl) benzonitrile | 2-hydroxy-5- (trifluoromethyl) benzonitrile (CAS: 142167- 36-0) | 527.3 [M + H]⁺ |
| 115 | 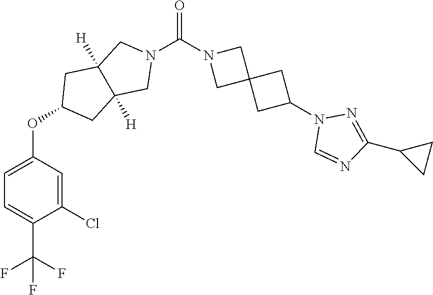 [SMILES: O=C(N1C[C@H]2C[C@H](Oc3ccc(C(F)(F)F)c(Cl)c3)C[C@H]2C1)N1CC2(CC(n3cnc(C4CC4)n3)C2)C1] | [6-(3-cyclopropyl- 1,2,4-triazol-1-yl)-2- azaspiro[3.3]heptan-2- yl]-[rac-(3aS,6aR)-5- [3-chloro-4- (trifluoromethyl) phenoxy]-3,3a,4,5,6,6a- hexahydro-1H- cyclopenta[c]pyrrol-2- yl]methanone | 3-chloro-4- (trifluoromethyl) phenol (CAS: 37900-81-5) | 536.2 [M + H]⁺ |
| 116 | 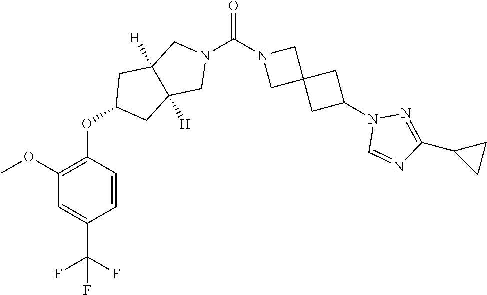 [SMILES: COc1cc(C(F)(F)F)ccc1O[C@H]1C[C@@H]2CN(C(=O)N3CC4(CC(n5cnc(C6CC6)n5)C4)C3)C[C@@H]2C1] | [6-(3-cyclopropyl- 1,2,4-triazol-1-yl)-2- azaspiro[3.3]heptan-2- yl]-[rac-(3aS,6aR)-5- [2-methoxy-4- (trifluoromethyl) phenoxy]-3,3a,4,5,6,6a- hexahydro-1H- cyclopenta[c]pyrrol-2- yl]methanone | 2-methoxy-4- (trifluoromethyl) phenol (CAS: 1027888-79-4) | 532.2 [M + H]⁺ |
| 117 | 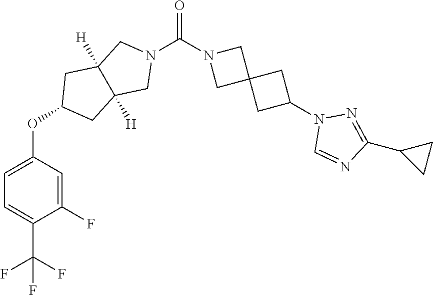 [SMILES: O=C(N1C[C@H]2C[C@H](Oc3ccc(C(F)(F)F)c(F)c3)C[C@H]2C1)N1CC2(CC(n3cnc(C4CC4)n3)C2)C1] | [6-(3-cyclopropyl- 1,2,4-triazol-1-yl)-2- azaspiro[3.3]heptan-2- yl]-[rac-(3aS,6aR)-5- [3-fluoro-4- (trifluoromethyl) phenoxy]-3,3a,4,5, 6,6a-hexahydro-1H- cyclopenta[c]pyrrol-2- yl]methanone | 3-fluoro-4- (trifluoromethyl) phenol (CAS: 219581-07-4) | 520.2 [M + H]⁺ |
| 118 | 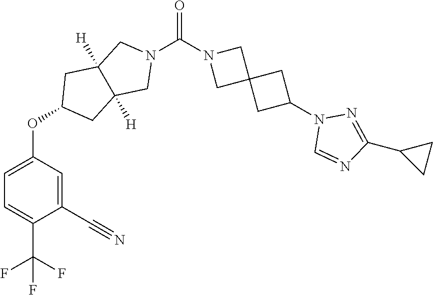 [SMILES: N#Cc1cc(O[C@H]2C[C@@H]3CN(C(=O)N4CC5(CC(n6cnc(C7CC7)n6)C5)C4)C[C@@H]3C2)ccc1C(F)(F)F] | 5-[rac-(3aS,6aR)-2- [6-(3-cyclopropyl- 1,2,4-triazol-1-yl)-2- azaspiro[3.3]heptane- 2-carbonyl]- 3,3a,4,5,6,6a- hexahydro-1H- cyclopenta[c]pyrrol-5- yl]oxy]-2- (trifluoromethyl) benzonitrile | 5-hydroxy-2- (trifluoromethyl) benzonitrile (CAS: 731002- 49-6) | 527.2 [M + H]⁺ |
| 119 | 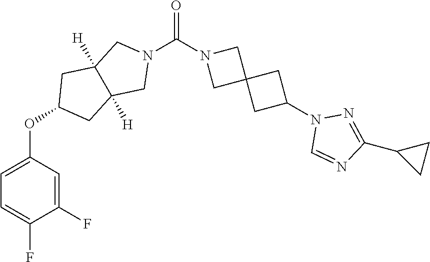 [SMILES: O=C(N1C[C@H]2C[C@H](Oc3ccc(F)c(F)c3)C[C@H]2C1)N1CC2(CC(n3cnc(C4CC4)n3)C2)C1] | [6-(3-cyclopropyl- 1,2,4-triazol-1-yl)-2- azaspiro[3.3]heptan-2- yl]-[rac-(3aS,6aR)-5- (3,4-difluorophenoxy)- 3,3a,4,5,6,6a- hexahydro-1H- cyclopenta[c]pyrrol-2- yl]methanone | 3,4- difluorophenol | 470.2 [M + H]⁺ |
| 120 | 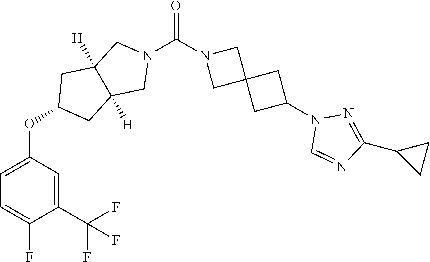 [SMILES: O=C(N1C[C@H]2C[C@H](Oc3ccc(F)c(C(F)(F)F)c3)C[C@H]2C1)N1CC2(CC(n3cnc(C4CC4)n3)C2)C1] | [6-(3-cyclopropyl- 1,2,4-triazol-1-yl)-2- azaspiro[3.3]heptan-2- yl]-[rac-(3aS,6aR)-5- [4-fluoro-3- (trifluoromethyl) phenoxy]-3,3a,4,5, 6,6a-hexahydro-1H- cyclopenta[c]pyrrol-2- yl]methanone | 4-fluoro-3- (trifluoromethyl) phenol | 520.2 [M + H]⁺ |
| 121 | 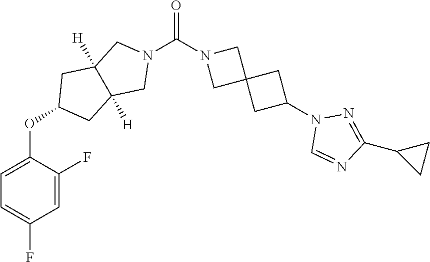 [SMILES: O=C(N1C[C@H]2C[C@H](Oc3ccc(F)cc3F)C[C@H]2C1)N1CC2(CC(n3cnc(C4CC4)n3)C2)C1] | [6-(3-cyclopropyl- 1,2,4-triazol-1-yl)-2- azaspiro[3.3]heptan-2- yl]-[rac-(3aS,6aR)-5- (2,4- difluorophenoxy)- 3,3a,4,5,6,6a- hexahydro-1H- cyclopenta[c]pyrrol-2- yl]methanone | 2,4- difluorophenol | 470.2 [M + H]⁺ |
| 122 | 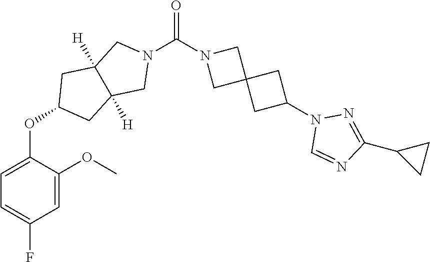 [SMILES: COc1cc(F)ccc1O[C@H]1C[C@@H]2CN(C(=O)N3CC4(CC(n5cnc(C6CC6)n5)C4)C3)C[C@@H]2C1] | [6-(3-cyclopropyl- 1,2,4-triazol-1-yl)-2- azaspiro[3.3]heptan-2- yl]-[rac-(3aS,6aR)-5- (4-fluoro-2-methoxy- phenoxy)- 3,3a,4,5,6,6a- hexahydro-1H- cyclopenta[c]pyrrol-2- yl]methanone | 4-fluoro-2- methoxy- phenol | 482.3 [M + H]⁺ |
| 123 | 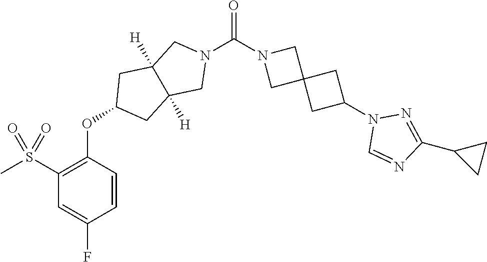 [SMILES: CS(=O)(=O)c1cc(F)ccc1O[C@H]1C[C@@H]2CN(C(=O)N3CC4(CC(n5cnc(C6CC6)n5)C4)C3)C[C@@H]2C1] | [6-(3-cyclopropyl- 1,2,4-triazol-1-yl)-2- azaspiro[3.3]heptan-2- yl]-[rac-(3aS,6aR)-5- (4-fluoro-2- methylsulfonyl- phenoxy)- 3,3a,4,5,6,6a- hexahydro-1H- cyclopenta[c]pyrrol-2- yl]methanone | 4-fluoro-2- methylsulfonyl- phenol (CAS: 398456-87-6) | 530.3 [M + H]⁺ |
| 124 | 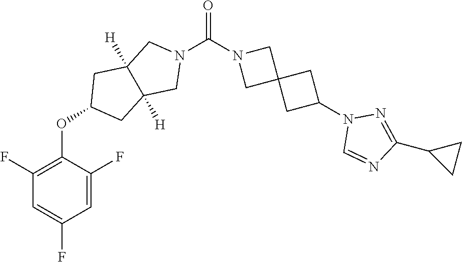 [SMILES: O=C(N1C[C@H]2C[C@H](Oc3c(F)cc(F)cc3F)C[C@H]2C1)N1CC2(CC(n3cnc(C4CC4)n3)C2)C1] | [6-(3-cyclopropyl- 1,2,4-triazol-1-yl)-2- azaspiro[3.3]heptan-2- yl]-[rac-(3aS,6aR)-5- (2,4,6- trifluorophenoxy)- 3,3a,4,5,6,6a- hexahydro-1H- cyclopenta[c]pyrrol-2- yl]methanone | 2,4,6- trifluorophenol (CAS: 2268- 17-9) | 488.2 [M + H]⁺ |
| 125 | 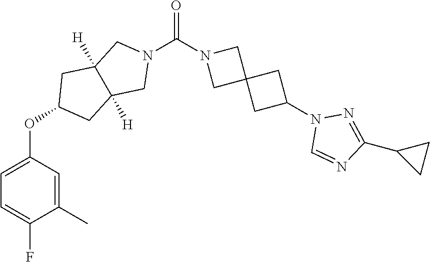 [SMILES: Cc1cc(O[C@H]2C[C@@H]3CN(C(=O)N4CC5(CC(n6cnc(C7CC7)n6)C5)C4)C[C@@H]3C2)ccc1F] | [6-(3-cyclopropyl- 1,2,4-triazol-1-yl)-2- azaspiro[3.3]heptan-2- yl]-[rac-(3aS,6aR)-5- (4-fluoro-3-methyl- phenoxy)- 3,3a,4,5,6,6a- hexahydro-1H- cyclopenta[c]pyrrol-2- yl]methanone | 4-fluoro-3- methylphenol (CAS: 452-70-0) | 466.2 [M + H]⁺ |
| 126 | 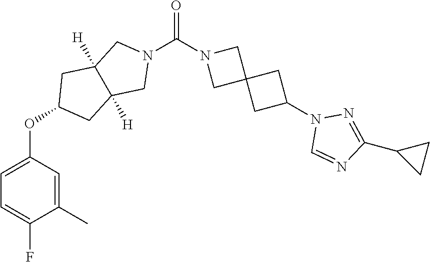 | [6-(3-cyclopropyl- 1,2,4-triazol-1-yl)-2- azaspiro[3.3]heptan-2- yl]-[rac-(3aS,6aR)-5- (4-fluoro-3-chloro- phenoxy)- 3,3a,4,5,6,6a- hexahydro-1H- cyclopenta[c]pyrrol-2- yl]methanone | 4-fluoro-3- chlorophenol (CAS: 2613- 23-2) | 486.2 [M + H]⁺ |
| 127 | 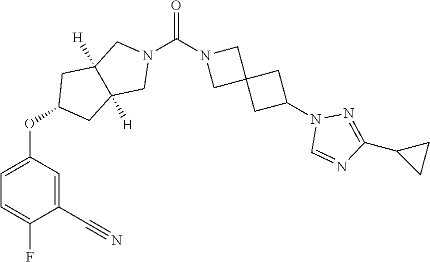 [SMILES: N#Cc1cc(O[C@H]2C[C@@H]3CN(C(=O)N4CC5(CC(n6cnc(C7CC7)n6)C5)C4)C[C@@H]3C2)ccc1F] | 2-fluoro-5-[[rac- (3aS,6aR)-2-[6-(3- cyclopropyl-1,2,4- triazol-1-yl)-2- azaspiro[3.3]heptane- 2-carbonyl]- 3,3a,4,5,6,6a- hexahydro-1H- cyclopenta[c]pyrrol-5- yl]oxy]benzonitrile | 2-fluoro-5- hydroxybenzen ecarbonitrile (CAS: 104798- 53-0) | 477.2 [M + H]⁺ |
| 128 | 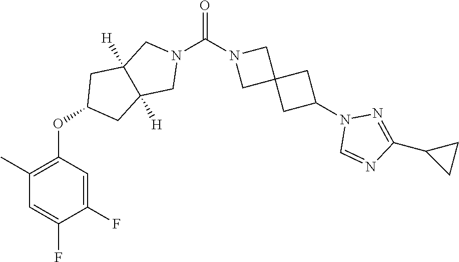 [SMILES: Cc1cc(F)c(F)cc1O[C@H]1C[C@@H]2CN(C(=O)N3CC4(CC(n5cnc(C6CC6)n5)C4)C3)C[C@@H]2C1] | [6-(3-cyclopropyl- 1,2,4-triazol-1-yl)-2- azaspiro[3.3]heptan-2- yl]-[rac-(3aS,6aR)-5- (4,5-difluoro-2- methyl-phenoxy)- 3,3a,4,5,6,6a- hexahydro-1H- cyclopenta[c]pyrrol-2- yl]methanone | 4,5-difluoro-2- methyl-phenol (CAS: 704884- 76-4) | 484.2 [M + H]⁺ |
| 129 | 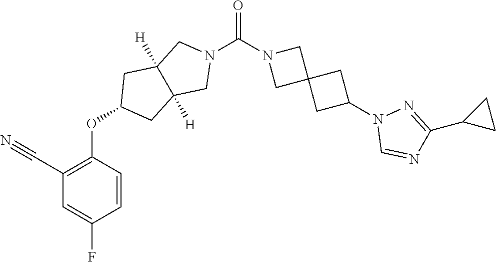 [SMILES: N#Cc1cc(F)ccc1O[C@H]1C[C@@H]2CN(C(=O)N3CC4(CC(n5cnc(C6CC6)n5)C4)C3)C[C@@H]2C1] | 5-fluoro-2-[[rac- (3aS,6aR)-2-[6-(3- cyclopropyl-1,2,4- triazol-1-yl)-2- azaspiro[3.3]heptane- 2-carbonyl]- 3,3a,4,5,6,6a- hexahydro-1H- cyclopenta[c]pyrrol-5- yl]oxy]benzonitrile | 5-fluoro-2- hydroxy- benzonitrile (CAS: 91407- 41-9) | 477.2 [M + H]⁺ |
| 130 | 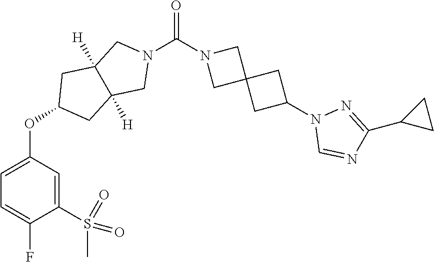 [SMILES: CS(=O)(=O)c1cc(O[C@H]2C[C@@H]3CN(C(=O)N4CC5(CC(n6cnc(C7CC7)n6)C5)C4)C[C@@H]3C2)ccc1F] | [6-(3-cyclopropyl- 1,2,4-triazol-1-yl)-2- azaspiro[3.3]heptan-2- yl]-[rac-(3aS,6aR)-5- (4-fluoro-3- methylsulfonyl- phenoxy)- 3,3a,4,5,6,6a- hexahydro-1H- cyclopenta[c]pyrrol-2- yl]methanone | 4-fluoro-3- methylsulfonyl- phenol (CAS: 2060020-74-6) | 530.2 [M + H]⁺ |
| 131 | 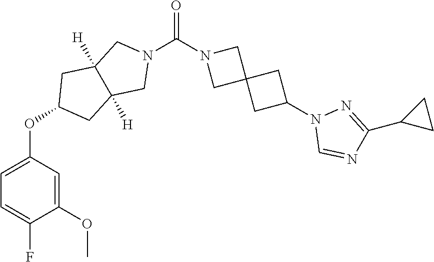 [SMILES: COc1cc(O[C@H]2C[C@@H]3CN(C(=O)N4CC5(CC(n6cnc(C7CC7)n6)C5)C4)C[C@@H]3C2)ccc1F] | [6-(3-cyclopropyl- 1,2,4-triazol-1-yl)-2- azaspiro[3.3]heptan-2- yl]-[rac-(3aS,6aR)-5- (4-fluoro-3-methoxy- phenoxy)- 3,3a,4,5,6,6a- hexahydro-1H- cyclopenta[c]pyrrol-2- yl]methanone | 4-fluoro-3- methoxyphenol (CAS: 117902-15-5) | 482.2 [M + H]⁺ |
| 132 | 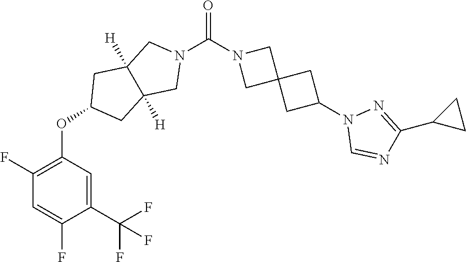 [SMILES: O=C(N1C[C@H]2C[C@H](Oc3cc(C(F)(F)F)c(F)cc3F)C[C@H]2C1)N1CC2(CC(n3cnc(C4CC4)n3)C2)C1] | [6-(3-cyclopropyl- 1,2,4-triazol-1-yl)-2- azaspiro[3.3]heptan-2- yl]-[rac-(3aS,6aR)-5- [2,4-difluoro-5- (trifluoromethyl) phenoxy]-3,3a,4,5,6,6a- hexahydro-1H- cyclopenta[c]pyrrol-2- yl]methanone | 2,4-difluoro-5- (trifluoromethyl) phenol (CAS: 1632283-20-5) | 538.2 [M + H]⁺ |
| 133 | 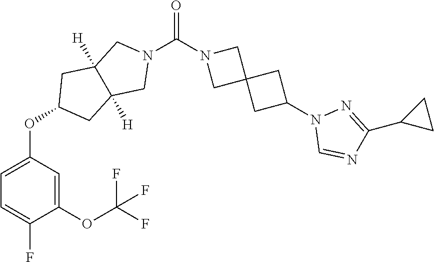 [SMILES: O=C(N1C[C@H]2C[C@H](Oc3ccc(F)c(OC(F)(F)F)c3)C[C@H]2C1)N1CC2(CC(n3cnc(C4CC4)n3)C2)C1] | [6-(3-cyclopropyl- 1,2,4-triazol-1-yl)-2- azaspiro[3.3]heptan-2- yl]-[rac-(3aS,6aR)-5- [4-fluoro-3- (trifluoromethoxy) phenoxy]-3,3a,4,5,6,6a- hexahydro-1H- cyclopenta[c]pyrrol-2- yl]methanone | 4-fluoro-3- (trifluoromethoxy) phenol (CAS: 61721- 07-1) | 536.2 [M + H]⁺ |
| 134 | 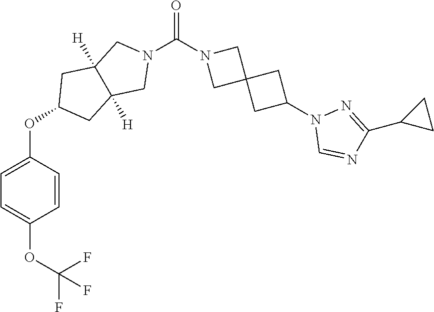 [SMILES: O=C(N1C[C@H]2C[C@H](Oc3ccc(OC(F)(F)F)cc3)C[C@H]2C1)N1CC2(CC(n3cnc(C4CC4)n3)C2)C1] | [6-(3-cyclopropyl- 1,2,4-triazol-1-yl)-2- azaspiro[3.3]heptan-2- yl]-[rac-(3aS,6aR)-5- [4- (trifluoromethoxy) phenoxy]-3,3a,4,5,6,6a- hexahydro-1H- cyclopenta[c]pyrrol-2- yl]methanone | 4- (trifluoromethoxy) phenol (CAS: 828-27- 3) | 518.2 [M + H]⁺ |
| 135 | 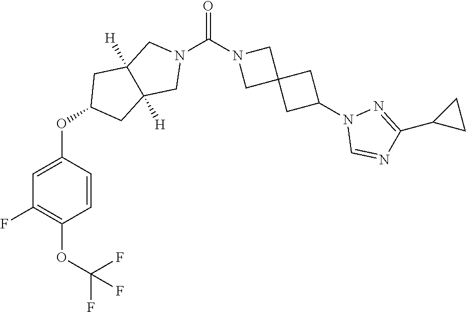 [SMILES: O=C(N1C[C@H]2C[C@H](Oc3ccc(OC(F)(F)F)c(F)c3)C[C@H]2C1)N1CC2(CC(n3cnc(C4CC4)n3)C2)C1] | [6-(3-cyclopropyl- 1,2,4-triazol-1-yl)-2- azaspiro[3.3]heptan-2- yl]-[rac-(3aS,6aR)-5- [3-fluoro-4- (trifluoromethoxy) phenoxy]-3,3a,4,5,6,6a- hexahydro-1H- cyclopenta[c]pyrrol-2- yl]methanone | 3-fluoro-4- (trifluoromethoxy) phenol (CAS: 177596- 38-2) | 536.2 [M + H]⁺ |

### Example 139

### [6-(4-cyclopropylimidazol-1-yl)-2-azaspiro[3.3]heptan-2-yl]−[3-[2-[2-(difluoromethyl)phenyl]ethynyl]azetidin-1-yl]methanone

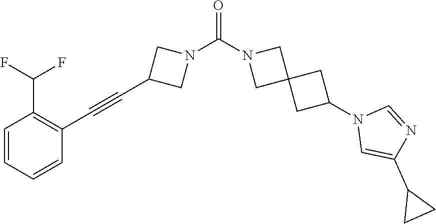 [SMILES: O=C(N1CC(C#Cc2ccccc2C(F)F)C1)N1CC2(CC(n3cnc(C4CC4)c3)C2)C1]

To a solution of 6-(4-cyclopropyl-1H-imidazol-1-yl)-2-azaspiro[3.3]heptane (A.2, free base) (35 mg, 172 μmol) in acetonitrile (1.98 mL) at 0° C. was added Hunig's base (63 mg, 85.1 μL, 487 μmol) followed by di(1H-1,2,4-triazol-1-yl)methanone (28.2 mg, 172 μmol) and the reaction mixture was stirred at RT for 1 h. Then Hunig's base (63 mg, 85.1 μL, 487 μmol, Eq: 4) was added, followed by 3-((2-(difluoromethyl)phenyl)ethynyl)azetidine (B.46) (25.3 mg, 122 μmol), as a colourless wax. MS (ESI): m/z=437.2 [M+H]⁺. In analogy to Example 139, Examples in the following table were generated, using the respective building blocks A.X and B.X.

| | | | | MS, |
|---|---|---|---|---|
| | | | Building | ESI: |
| Ex. | Structure | Systematic Name | Blocks | m/z |
| 141 | 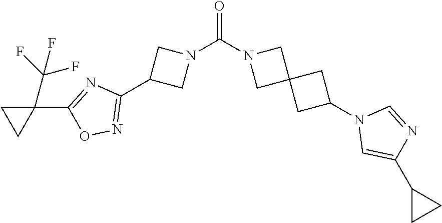 [SMILES: O=C(N1CC(c2noc(C3(C(F)(F)F)CC3)n2)C1)N1CC2(CC(n3cnc(C4CC4)c3)C2)C1] | [6-(4- cyclopropylimidazol-1- yl)-2- azaspiro[3.3]heptan-2- yl]-[3-[5-[1- (trifluoromethyl) cyclopropyl]- 1,2,4-oxadiazol- 3-ylazetidin-1- yl]methanone | A.2 and B.53 | 463.3 [M + H]⁺ |
| 144 | 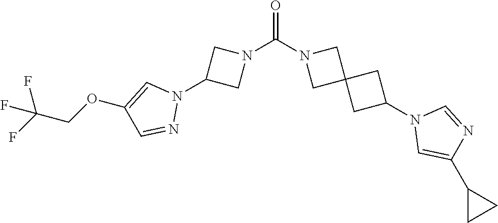 [SMILES: O=C(N1CC(n2cc(OCC(F)(F)F)cn2)C1)N1CC2(CC(n3cnc(C4CC4)c3)C2)C1] | [6-(4- cyclopropylimidazol-1- yl)-2- azaspiro[3.3]heptan-2- yl]-[3-[4-(2,2,2- trifluoroethoxy)pyrazol- 1-yl]azetidin-1- yl]methanone | A.2 and B.54 | 451.2 [M + H]⁺ |

### Example 142

### [6-(4-cyclopropylimidazol-1-yl)-2-azaspiro[3.3]heptan-2-yl]−[5-methyl-6-[(1-methylcyclopropyl)methoxy]-3-pyridyl]methanone

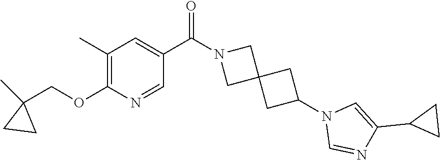 [SMILES: Cc1cc(C(=O)N2CC3(CC(n4cnc(C5CC5)c4)C3)C2)cnc1OCC1(C)CC1]

To a stirred suspension of NaH (6.25 mg, 156 μmol) in DMF (574 μL) under inert conditions and at room temperature (1-methylcyclopropyl)methanol (13.5 mg, 156 μmol) was added and stirred for 15 min. Thereafter a solution of (6-(4-cyclopropyl-1H-imidazol-1-yl)-2-azaspiro[3.3]heptan-2-yl)(6-fluoro-5-methylpyridin-3-yl)methanone (26.6 mg, 78.1 μmol) in DMF (574 μL) was added and the reaction stirred at RT for 5.5 h. The mixture was diluted with EtOAc and washed with water, the aqueous phase was then extracted with EtOAc. The combined organics were washed with brine, dried over sodium sulfate, filtered and concentrated in vacuo. The residue was purified through flash chromatography eluting with DCM:MeOH 100:0 to 95:10. The title compound (8.8 mg, 20.6 μmol, 26.3% yield) was obtained as a sticky colourless oil. MS (ESI): m/z=407.4 [M+H]⁺

### Step a) (6-(4-cyclopropyl-]H-imidazol-1-yl)-2-azaspiro[3.3]heptan-2-yl)(6-fluoro-5-methylpyridin-3-yl)methanone

To a stirred solution of 6-(4-cyclopropyl-1H-imidazol-1-yl)-2-azaspiro[3.3]heptane (A.2) bis(4-methylbenzenesulfonate) (150 mg, 274 μmol) at room temperature under an inert argon atmosphere in DMF (1.4 mL), 6-fluoro-5-methylnicotinic acid (40.4 mg, 260 μmol), Hunig's base (142 mg, 191 μL, 1.1 mmol) and finally HATU (115 mg, 301 μmol) were added and the solution stirred at room temperature overnight. The mixture was diluted with EtOAc (5 mL), washed with water and extracted with EtOAc. The organic layers were combined and washed with sodium bicarbonate, brine, dried over sodium sulfate, filtered and concentrated in vacuo yielding the crude as a yellow oil. Purification by reverse phase HPLC to yield the title compound (26.6 mg, 78.1 μmol, 28.5% yield), as a colourless liquid. MS (ESI): m/z=341.2 [M+H]⁺

In analogy to Example 142, Examples in the following table were generated, using the respective building blocks A.X

| | | | | MS, |
|---|---|---|---|---|
| | | | Building | ESI: |
| Ex. | Structure | Systematic Name | Blocks | m/z |
| 143 | 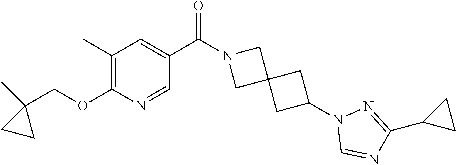 [SMILES: Cc1cc(C(=O)N2CC3(CC(n4cnc(C5CC5)n4)C3)C2)cnc1OCC1(C)CC1] | [6-(3-cyclopropyl- 1,2,4-triazol-1-yl)-2- azaspiro[3.3]heptan-2- yl]-[5-methyl-6-[(1- methylcyclopropyl) methoxy]-3- pyridyl]methanone | A.1 | 408.4 [M + H]⁺ |

### Example 146 [4-(5-tert-butyl-1,2,4-oxadiazol-3-yl)phenyl]−[6-(4-methylpyrazol-1-yl)-2-azaspiro[3.3]heptan-2-yl]methanone

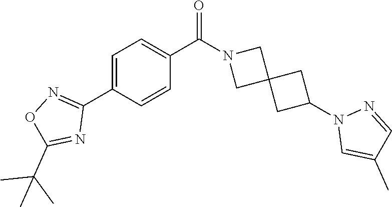 [SMILES: Cc1cnn(C2CC3(C2)CN(C(=O)c2ccc(-c4noc(C(C)(C)C)n4)cc2)C3)c1]

A mixture of T3P (1.3 mL, 1.34 mmol), DIEA (288 mg, 2.23 mmol), 4-(5-tert-butyl-1,2,4-oxadiazol-3-yl)benzoic acid (CAS: 1119452-72-0) (110 mg, 0.450 mmol) and 6-(4-methylpyrazol-1-yl)-2-azaspiro[3.3]heptane; 2,2,2-trifluoroacetic acid (A.4) (130 mg, 0.450 mmol) in DMF (6.5 mL) was stirred at 20° C. for 12 h. The mixture was purified by prep-HPLC (FA) and lyophilized to give the title compound (18.9 mg, 0.050 mmol, 10.2% yield) as white solid. MS (ESI): m/z=406.4 [M+H]⁺

In analogy to Example 146, Examples in the following table were generated, using the respective building blocks A.X and B.X

| | | | | MS, |
|---|---|---|---|---|
| | | | Building | ESI: |
| Ex. | Structure | Systematic Name | Blocks | m/z |
| 147 | 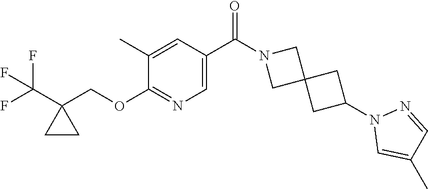 [SMILES: Cc1cnn(C2CC3(C2)CN(C(=O)c2cnc(OCC4(C(F)(F)F)CC4)c(C)c2)C3)c1] | [6-(4-methylpyrazol-1- yl)-2- azaspiro[3.3]heptan-2- yl]-[5-methyl-6-[[1- (trifluoromethyl) cyclopropyl] methoxy]-3- pyridyl]methanone | A.4 and B.3 | 435.6 [M + H]⁺ |
| 148 | 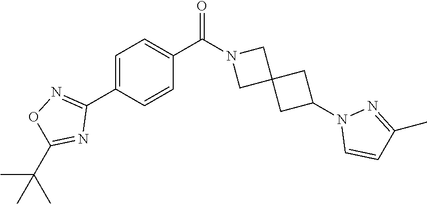 [SMILES: Cc1ccn(C2CC3(C2)CN(C(=O)c2ccc(-c4noc(C(C)(C)C)n4)cc2)C3)n1] | [4-(5-tert-butyl-1,2,4- oxadiazol-3-yl)phenyl]- [6-(3-methylpyrazol-1- yl)-2- azaspiro[3.3]heptan-2- yl]methanone | A.5 and CAS:111 9452-72- 0 | 406.4 [M + H]⁺ |
| 151 | 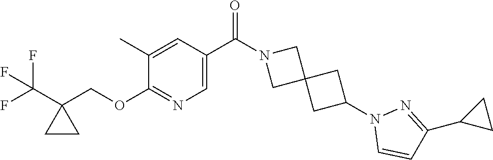 [SMILES: Cc1cc(C(=O)N2CC3(CC(n4ccc(C5CC5)n4)C3)C2)cnc1OCC1(C(F)(F)F)CC1] | [6-(3- cyclopropylpyrazol-1- yl)-2- azaspiro[3.3]heptan-2- yl]-[5-methyl-6-[[1- (trifluoromethyl) cyclopropyl] methoxy]-3- pyridyl]methanone | A.6 and B.3 | 461.5 [M + H]⁺ |
| 152 | 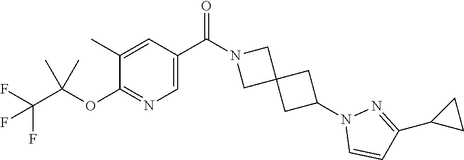 [SMILES: Cc1cc(C(=O)N2CC3(CC(n4ccc(C5CC5)n4)C3)C2)cnc1OC(C)(C)C(F)(F)F] | [6-(3- cyclopropylpyrazol-1- yl)-2- azaspiro[3.3]heptan-2- yl]-[5-methyl-6-(2,2,2- trifluoro-1,1-dimethyl- ethoxy)-3- pyridyl]methanone | A.6 and B.56 | 449.5 [M + H]⁺ |
| 174 | 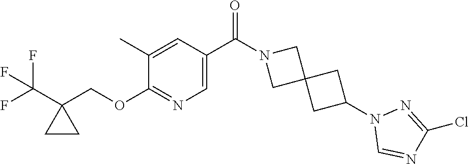 [SMILES: Cc1cc(C(=O)N2CC3(CC(n4cnc(Cl)n4)C3)C2)cnc1OCC1(C(F)(F)F)CC1] | [6-(3-chloro-1,2,4- triazol-1-yl)-2- azaspiro[3.3]heptan-2- yl]-[5-methyl-6-[[1- (trifluoromethyl) cyclopropyl] methoxy]-3- pyridyl]methanone | A.8 and B.3 | 456.3 [M + H]⁺ |
| 175 | 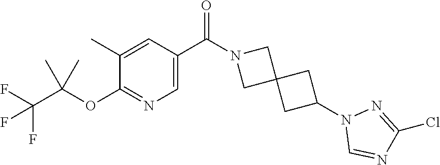 [SMILES: Cc1cc(C(=O)N2CC3(CC(n4cnc(Cl)n4)C3)C2)cnc1OC(C)(C)C(F)(F)F] | [6-(3-chloro-1,2,4- triazol-1-yl)-2- azaspiro[3.3]heptan-2- yl]-[5-methyl-6-(2,2,2- trifluoro-1,1-dimethyl- ethoxy)-3- pyridyl]methanone | A.8 and B.56 | 444.4 [M + H]⁺ |

### Example 149

### [6-(3-cyclopropyl-1,2,4-triazol-1-yl)-2-azaspiro13.31heptan-2-yl]−[8-[[1-(trifluoromethyl)cyclopropyl]methoxy]-5-azaspiro[2.5]octan-5-yl]methanone

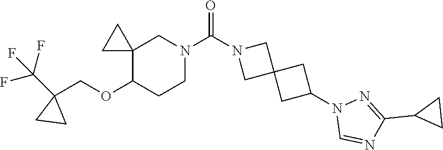 [SMILES: O=C(N1CC2(CC(n3cnc(C4CC4)n3)C2)C1)N1CCC(OCC2(C(F)(F)F)CC2)C2(CC2)C1]

To a solution of 6-(3-cyclopropyl-1,2,4-triazol-1-yl)-2-azaspiro[3.3]heptane; 2,2,2-trifluoroacetic acid (A.1) (55.0 mg, 0.170 mmol) and DIPEA (67.0 mg, 0.520 mmol) in ACN (2 mL) was added (4-nitrophenyl) 3-ethyl-3-methyl-4-[[1-(trifluoromethyl)cyclopropyl]methoxy]piperidine-1l-carboxylate (55.8 mg, 0.130 mmol), then the mixture was stirred at 90° C. for 12 h. The mixture was concentrated and the residue was purified by prep-HPLC (0.225% v/v FA) and lyophilized to give the title compound (10.6 mg, 0.020 mmol, 12.1% yield) as a yellow foam. MS (ESI): m/z=480.2 [M+H]⁺

### Step a) tert-butyl 8-[[ ]-(trifluoromethyl)cyclopropyl]methoxy]-5-azaspiro[2.5]octane-5-carboxylate

To a solution of tert-butyl 8-hydroxy-5-azaspiro[2.5]octane-5-carboxylate (CAS: 955028-95-2) (100 mg, 0.440 mmol) in DMF (2 mL) was added NaH (35.2 mg, 0.880 mmol) at 0° C. The reaction mixture was stirred for 30 min, then added the [1-(trifluoromethyl)cyclopropyl]methyl methanesulfonate (CAS: 1262400-04-3) (144 mg, 0.660 mmol), the mixture was stirred at 50° C. for 12 h. The mixture was poured into sat. aq. NH₄C₁ solution (10 mL) and extracted with EtOAc (3 mL three times), the combine organic phase was washed with brine and dried over Na₂SO₄, then concentrated, the residue was purified by silica gel column (PE:EtOAc=30:1 to 20:1) to give the title compound (54 mg, 0.150 mmol, 35.1% yield) as light yellow oil. MS (ESI): m/z=250.4 [M-C₄H₈+H]⁺

### Step b) 8-[[ ]-(trifluoromethyl)cyclopropyl]methoxy]-5-azaspiro[2.5]octane trifluoroacetic acid salt

To a solution of tert-butyl 8-[[1-(trifluoromethyl)cyclopropyl]methoxy]-5-azaspiro[2.5]octane-5-carboxylate (50.0 mg, 0.140 mmol) in DCM (1 mL) was added TFA (0.1 mL, 0.140 mmol) in DCM (1 mL) at 0° C., then the mixture was stirred at 25° C. for 1 h. The reaction mixture was concentrated under reduced pressure to give the title compound (50 mg, 0.140 mmol, 96.2% yield) as a crude yellow oil which was used in the next step without further purification. MS (ESI): m/z=250.1 [M+H]⁺

### Step c) (4-nitrophenyl) 3-ethyl-3-methyl-4-[[(trifluoromethyl)cyclopropyl]methoxy]piperidine-]-carboxylate

To a solution of 4-nitrophenyl chloroformate (30.5 mg, 0.150 mmol) in ACN (1.25 mL) was added DIPEA (44.4 mg, 0.340 mmol) with the temperature kept at 0° C., then 8-[[1-(trifluoromethyl)cyclopropyl]methoxy]-5-azaspiro[2.5]octane trifluoroacetic acid salt (50.0 mg, 0.140 mmol) was added. The reaction mixture was stirred at 90° C. for 12 h. The reaction mixture was concentrated under reduced pressure and purified by prep-TLC (PE: EtOAc=2: 1) to give the title compound (52.0 mg, 0.130 mmol, 91.2% yield) as yellow oil. MS (ESI): m/z=415.4 [M+H]⁺

In analogy to Example 150, Examples in the following table were generated, using the respective building blocks A.X and B.X

| | | | | MS, |
|---|---|---|---|---|
| | | | Building | ESI: |
| Ex. | Structure | Systematic Name | Blocks | m/z |
| 150 | 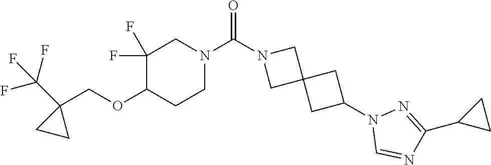 [SMILES: O=C(N1CC2(CC(n3cnc(C4CC4)n3)C2)C1)N1CCC(OCC2(C(F)(F)F)CC2)C(F)(F)C1] | [6-(3-cyclopropyl- 1,2,4-triazol-1-yl)-2- azaspiro[3.3]heptan- 2-yl]-[3,3-difluoro- 4-[[1- (trifluoromethyl) cyclopropyl]methoxy]-1- piperidyl]methanone | tert-butyl 3,3- difluoro-4- hydroxy- piperidine- 1-carboxylate (CAS: 1209780- 71-1) | 490.3 [M + H]⁺ |

### Example 153

### [6-(3-cyclopropyl-1,2,4-triazol-1-yl)-2-azaspiro[3.3]heptan-2-yl]−[5-(trifluoromethyl)-6-[[1-(trifluoromethyl)cyclopropyl]methoxy]-3-pyridyl]methanone

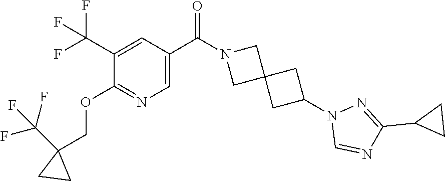 [SMILES: O=C(c1cnc(OCC2(C(F)(F)F)CC2)c(C(F)(F)F)c1)N1CC2(CC(n3cnc(C4CC4)n3)C2)C1]

To a solution of 5-(trifluoromethyl)-6-((1-(trifluoromethyl)cyclopropyl)methoxy)nicotinic acid (100 mg, 304 μmol) in dry DMF (2 mL) was added DIPEA (157 mg, 212 μl, 1.22 mmol) and HATU (121 mg, 319 μmol), the reaction mixture was stirred at RT for 10 min followed by addition of 6-(3-cyclopropyl-1H-1,2,4-triazol-1-yl)-2-azaspiro[3.3]heptane bis(4-methylbenzenesulfonate) (A.1, tosylate salt) (183 mg, 334 μmol). The reaction mixture was stirred at RT for 3 h. The crude reaction mixture was directly submitted for reversed-phase HPLC purification to yield the title compound (78.9 mg, 49.4% yield). MS (ESI): m/z=516.3 [M+H]⁺

### Step a) 5-(trifluoromethyl)-6-((1-(trifluoromethyl)cyclopropyl)methoxy)nicotinic acid

To a solution of (1-(trifluoromethyl)cyclopropyl)methanol (342 mg, 2.44 mmol) in dry DMSO (5 ml) under an inert atmosphere was added potassium tert-butoxide (547 mg, 4.88 mmol) and the reaction mixture was then stirred at RT for 5 min followed by addition of 6-chloro-5-(trifluoromethyl)nicotinic acid (CAS: 1110782-41-6) (500 mg, 2.22 mmol) after which the reaction was stirred at RT for 10 min. The reaction was then stirred at RT for 1 h. Addition of potassium tert-butoxide (124 mg, 1.11 mmol) and the reaction mixture was again stirred at RT for 1 h. The reaction mixture was then partitioned between ethyl acetate and aq. HCl 1 N solution. The organic phase was collected and the aqueous phase was back-extracted with ethyl acetate. The combined organic phases were dried over sodium sulfate and concentrated in vacuo. The crude solution (residual DMSO) was then poured into a small volume of aq. HCl 1M, and a precipitation occured to yield a gummy solid. The whole fraction was then evaporated down to dryness using a centrifugal evaporator to yield the crude title compound, which was used in the next step without further purification (687 mg, 84.7% yield). MS (ESI): m/z=330.1 [M+H]⁺

### Example 154

### [6-(3-cyclopropyl-1,2,4-triazol-1-yl)-2-azaspiro[3.3]heptan-2-yl]−[5-(oxetan-3-yl)-6-[[1-(trifluoromethyl)cyclopropyl]methoxy]-3-pyridyl]methanone

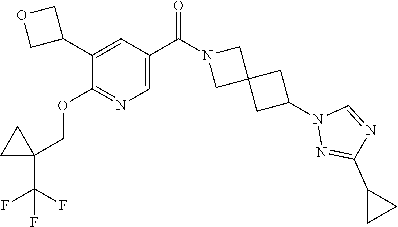 [SMILES: O=C(c1cnc(OCC2(C(F)(F)F)CC2)c(C2COC2)c1)N1CC2(CC(n3cnc(C4CC4)n3)C2)C1]

To a solution of 5-(oxetan-3-yl)-6-((1-(trifluoromethyl)cyclopropyl)methoxy)nicotinic acid (60 mg, 189 μmol) in dry DMF (1 mL) was added DIPEA (85.5 mg, 116 μL, 662 μmol) and HATU (75.5 mg, 199 μmol) after which the reaction mixture was stirred at RT for 10 min followed by addition of 6-(3-cyclopropyl-1H-1,2,4-triazol-1-yl)-2-azaspiro[3.3]heptane 2,2,2-trifluoroacetate (A.1) (66.2 mg, 208 μmol). The reaction mixture was then stirred at RT for 18 h. The crude reaction solution was directly purified by reversed-phase HPLC to yield 77.2 mg of the desired product (85% purity). The previous fraction was again purified by SFC to yield 52.4 mg of the title compound. MS (ESI): m/z=504.3 [M+H]⁺

### Step a) methyl 5-bromo-6-((1-(trifluoromethyl)cyclopropyl)methoxy)nicotinate

To a solution of (1-(trifluoromethyl)cyclopropyl)methanol (294 mg, 2.1 mmol) in dry DMF (11 mL) under an inert atmosphere was added NaH (83.8 mg, 2.1 mmol) and the reaction mixture was then stirred at RT for 5 min followed by addition of methyl 5-bromo-6-chloronicotinate (500 mg, 2 mmol) after which the reaction was stirred at RT for 10 min. The reaction was then stirred at RT for 18 h. The reaction mixture was quenched by addition of few drops of sat. aq. NH₄Cl and the reaction mixture was diluted with ethyl acetate followed by washing with aq. NaHCO₃ 1 M solution. The organic phase was collected and the aqueous phase was back-extracted with ethyl acetate. The combined organic phases were dried over sodium sulfate and evaporated down to dryness. The crude residue was purified by flash chromatography, eluting with a mixture of heptane and ethyl acetate (5% to 50%) to yield the title compound (577 mg, 80% yield). MS (ESI): m/z=354.1 [M+H]⁺

### Step b) methyl 5-(oxetan-3-yl)-6-((1-(trifluoromethyl)cyclopropyl)methoxy)nicotinate

To a solution of methyl 5-bromo-6-((1-(trifluoromethyl)cyclopropyl)methoxy)nicotinate (540 mg, 1.52 mmol) in dry DME (12 mL) under an inert atmosphere was added 3-bromooxetane (313 mg, 2.29 mmol), tris(trimethylsilyl)silane (379 mg, 470 μL, 1.52 mmol), (Ir[dF(CF₃)ppy]₂(dtbpy))PF₆ (17.1 mg, 15.2 μmol), sodium carbonate (323 mg, 3.05 mmol). A suspension of 16.8 mg nickel(II) chloride ethylene glycol dimethyl ether complex and 20.5 mg 4,4-di-tert-butyl-2,2′-dipyridyl in 1 mL of dry DME was stirred at RT under an inert atmosphere for 10 min and 0.1 mL of the stirred suspension was added to the previous reaction mixture after which the reaction was stirred at RT under Blue LED irradiation for 18 h. The reaction mixture was diluted with ethyl acetate and extracted with aq. sol. Na₂CO₃ 1 M, the organic phase was collected and the aqueous phase was back-extracted with ethyl acetate. The combined organic phases were dried over sodium sulfate and evaporated down to dryness. The crude material was purified by flash chromatography eluting with a mixture of heptane and ethyl acetate (5% to 60%) to yield the title compound (268 mg, 50% yield). MS (ESI): m/z=332.1 [M+H]⁺

### Step c) 5-(oxetan-3-yl)-6-((1-(trifluoromethyl)cyclopropyl)methoxy)nicotinic acid

To a solution of methyl 5-(oxetan-3-yl)-6-((1-(trifluoromethyl)cyclopropyl)methoxy)nicotinate (268 mg, 809 μmol) in methanol (5 mL) was added NaOH 5.0M aq. solution (324 μL, 1.62 mmol) and the reaction mixture was stirred at RT for 18 h. The reaction mixture was then partitioned between ethyl acetate and aq. sol. HCl 0.1 M, the organic phase was collected and the aqueous phase was back-extracted with ethyl acetate. The combined organic phases were dried over sodium sulfate and evaporated down to dryness to yield the title compound (244 mg) which was used without further purification. MS (ESI): m/z=318.1 [M+H]⁺

### Example 155

### [6-(3-cyclopropyl-1,2,4-triazol-1-yl)-2-azaspiro[3.3]heptan-2-yl]−[4-[[1-(trifluoromethyl)cyclopropyl]methoxymethyl]-1-bicyclo[2.2.2]octanyl]methanone

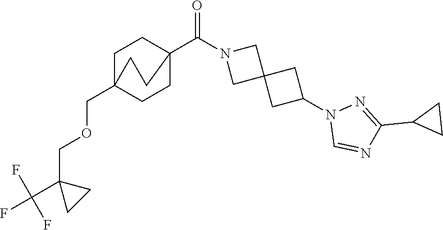 [SMILES: O=C(N1CC2(CC(n3cnc(C4CC4)n3)C2)C1)C12CCC(COCC3(C(F)(F)F)CC3)(CC1)CC2]

To a solution of [6-(3-cyclopropyl-1,2,4-triazol-1-yl)-2-azaspiro[3.3]heptan-2-yl]−[1-(hydroxymethyl)-4-bicyclo[2.2.2]octanyl]methanone (30.0 mg, 0.08 mmol) in DMF (0.5 mL) was added NaH (1.94 mg, 0.08 mmol) at 0° C. and the reaction mixture was stirred for 30 min. [1-(trifluoromethyl)cyclopropyl]methyl methanesulfonate (26.5 mg, 0.12 mmol), the mixture was stirred at 60° C. for 12 h. The reaction mixture was quenched by addition of sat. aq. NH₄C₁ (10 mL) solution at 0° C., and then extracted with EtOAc (10 mL×3). The combined organic phase was washed with brine (10 mL), dried over Na₂SO₄, concentrated in vacuo and purified by prep-HPLC (0.225% v/v FA) then lyophilized to give the title compound (4.1 mg, 0.01 mmol, 10% yield) as a white solid. MS (ESI): m/z=493.3 [M+H]⁺

### Step a) [6-(3-cyclopropyl-1,2,4-triazol-1-yl)-2-azaspiro[3.3]heptan-2-yl]−[ ]-(hydroxymethyl)-4-bicyclo[2.2.2]octanyl]methanone

A solution of methyl 1-(hydroxymethyl)bicyclo[2.2.2]octane-4-carboxylate (CAS: 94994-15-7) (40.0 mg, 0.2 mmol), 6-(3-cyclopropyl-1,2,4-triazol-1-yl)-2-azaspiro[3.3]heptane (A.1) (49.5 mg, 0.24 mmol) and 1,3,4,6,7,8-hexahydro-1H-pyrimido[1,2-a]pyrimidine (28.1 mg, 0.2 mmol) in THF (2 mL) was stirred at 75° C. for 16 h. The residue was concentrated under reduced pressure and purified by reverse flash chromatography to give the title compound (43.0 mg, 0.12 mmol, 57.5% yield) as a yellow oil. MS (ESI): m/z=371.2 [M+H]⁺

In analogy to Example 155, the following Examples were generated using the respective heteroaryl building block in Step a).

| | | | | MS, |
|---|---|---|---|---|
| | | | Building | ESI: |
| Ex. | Structure | Systematic Name | Blocks | m/z |
| 161 | 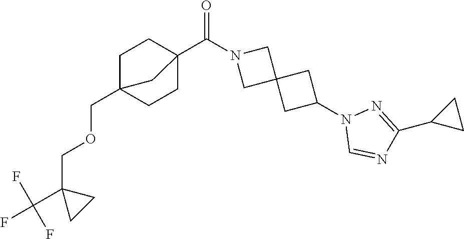 [SMILES: O=C(N1CC2(CC(n3cnc(C4CC4)n3)C2)C1)C12CCC(COCC3(C(F)(F)F)CC3)(CC1)C2] | [6-(3-cyclopropyl- 1,2,4-triazol-1-yl)-2- azaspiro[3.3]heptan- 2-yl]-[4-[[1- (trifluoromethyl) cyclopropyl] methoxymethyl] norbornan-1- yl]methanone | methyl 4- (hydroxymethyl) norbornane-1- carboxylate (CAS: 1350821- 95-2) | 479.3 [M + H]⁺ |

### Example 156

### [4-(5-tert-butyl-1,2,4-oxadiazol-3-yl)phenyl]−[6-(3-ethyl-1,2,4-triazol-1-yl)-2-azaspiro[3.3] heptan-2-yl]methanone

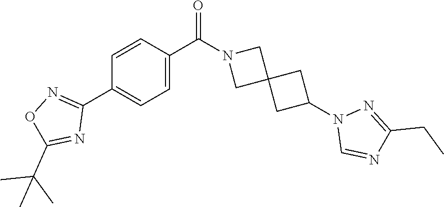 [SMILES: CCc1ncn(C2CC3(C2)CN(C(=O)c2ccc(-c4noc(C(C)(C)C)n4)cc2)C3)n1]

To a solution of tert-butyl 6-(3-ethyl-1H-1,2,4-triazol-1-yl)-2-azaspiro[3.3]heptane-2-carboxylate (160 mg, 547 μmol) in DCM (4.45 mL) was added TFA (624 mg, 422 μL 5.47 mmol). The mixture was stirred at room temperature for 2 h. The solvent was removed under reduced pressure, TFA was coevaporated with toluene. The TFA salt was redissolved in DMF (4.45 mL). DIPEA (424 mg, 573 μL, 3.28 mmol) was added followed by 4-(5-(tert-butyl)-1,2,4-oxadiazol-3-yl)benzoic acid (135 mg, 547 μmol) and 2,4,6-tripropyl-1,3,5,2,4,6-trioxatriphosphinane 2,4,6-trioxide (1.39 g, 995 μL, 2.19 mmol). The mixture was stirred for 15 h at room temperature. The mixture was diluted with ethyl acetate and washed with water. The aqueous phase was extracted twice with ethyl acetate, the organic phases were washed with water and brine. The combined organic phases were dried over MgSO₄ and the solvent evaporated under reduced pressure. The crude material was initially purified by column chromatography eluting with DCM:methanol (10% methanol). The phases containing the product were submitted to rpHPLC for purification to give the title compound (49 mg, 114 μmol, 20.9% yield) as a white powder. MS (ESI): m/z=412.3 [M+H]⁺.

### Step a) [6-(3-cyclopropyl-1, 2, 4-triazol-1-yl)-2-azaspiro[3.3]heptan-2-yl]−[ ]-(hydroxymethyl)-4-bicyclo[2.2.2]octanyl]methanone

To a solution of tert-butyl 6-((methylsulfonyl)oxy)-2-azaspiro[3.3]heptane-2-carboxylate (500 mg, 1.72 mmol) in DMF (17.2 mL) was added cesium carbonate (3.35 g, 10.3 mmol) followed by 3-ethyl-eH-1,2,4-triazole (250 mg, 2.57 mmol). The mixture was stirred at 100 C for 24 h. The mixture was diluted with ethyl acetate and washed with water. The aqueous phase was extracted with ethyl acetate twice, and the combined organic phase concentrated. The crude was purified by SFC to yield the title compound (160 mg, 547 μmol, 21.3% yield) was obtained as a colorless oil. MS (ESI): m/z=293.3 [M+H]⁺.

In analogy to Example 156, the following Examples were generated using the respective heteroaryl building block in Step a).

| | | | | MS, |
|---|---|---|---|---|
| | | | Building | ESI: |
| Ex. | Structure | Systematic Name | Block | m/z |
| 157 | 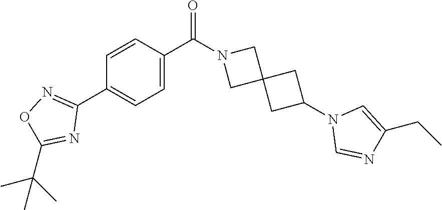 [SMILES: CCc1cn(C2CC3(C2)CN(C(=O)c2ccc(-c4noc(C(C)(C)C)n4)cc2)C3)cn1] | [4-(5-tert-butyl- 1,2,4-oxadiazol-3- yl)phenyl]-[6-(4- ethylimidazol-1-yl)-2- azaspiro[3.3]heptan- 2-yl]methanone | 4-ethyl-1H- imidazole | 420.3 [M + H]⁺ |
| 158 | 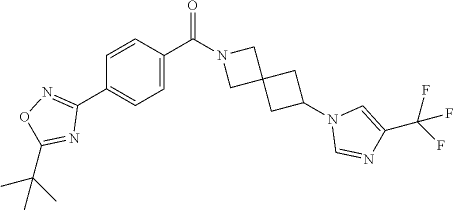 [SMILES: CC(C)(C)c1nc(-c2ccc(C(=O)N3CC4(CC(n5cnc(C(F)(F)F)c5)C4)C3)cc2)no1] | [4-(5-tert-butyl- 1,2,4-oxadiazol-3- yl)phenyl]-[6-[4- (trifluoromethyl) imidazol-1-yl]-2- azaspiro[3.3]heptan- 2-yl]methanone | 4- trifluoromethyl- 1H-imidazole (CAS: 33468-69-8) | 460.3 [M + H]⁺ |
| 160 | 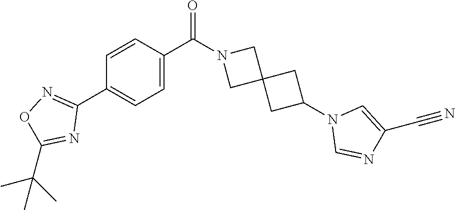 [SMILES: CC(C)(C)c1nc(-c2ccc(C(=O)N3CC4(CC(n5cnc(C#N)c5)C4)C3)cc2)no1] | 1-[2-[4-(5-tert-butyl- 1,2,4-oxadiazol-3- yl)benzoyl]-2- azaspiro[3.3]heptan- 6-yl]imidazole-4- carbonitrile | 1H- imidazole- 4- carbonitrile (CAS: 57090-88-7) | 417.2 [M + H]⁺ |

### Example 173

### [6-(3-cyclopropyl-1,2,4-triazol-1-yl)-2-azaspiro[3.3]heptan-2-yl]−[3-[[1-(trifluoromethyl)cyclopropyl]methoxymethyl]cyclobutyl]methanone

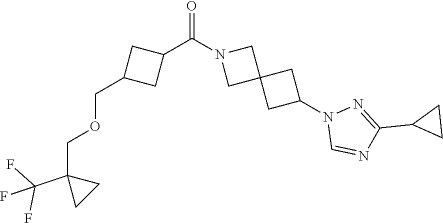 [SMILES: O=C(C1CC(COCC2(C(F)(F)F)CC2)C1)N1CC2(CC(n3cnc(C4CC4)n3)C2)C1]

To a solution of [6-(3-cyclopropyl-1,2,4-triazol-1-yl)-2-azaspiro[3.3]heptan-2-yl]−[3-(hydroxymethyl)cyclobutyl]methanone (30.0 mg, 0.09 mmol) in DMF (1 mL) was added NaH (2.28 mg, 0.09 mmol) at 0° C. and stirred for 30 min. Then a solution of [1-(trifluoromethyl)cyclopropyl]methyl methanesulfonate (31.0 mg, 0.14 mmol) in DMF (1 mL) was added and the reaction was stirred at 60° C. for 16 h. The reaction mixture was quenched by addition of sat, aq. NH₄Cl solution (10 mL) at 0° C. The mixture was extracted with EtOAc (10 mL×3). The combined organic phase was washed with brine (10 mL), dried over Na₂SO₄, concentrated in vacuo and purified by prep-HPLC (0.225% v/v FA) then lyophilized to give the title compound (6.5 mg, 0.01 mmol, 15% yield) as a yellow oil. MS (ESI): m/z=439.2 [M+H]⁺

### Step a) [6-(3-cyclopropyl-1,2,4-triazol-1-yl)-2-azaspiro[3.3]heptan-2-yl]−[3-(hydroxymethyl)cyclobutyl] methanone

A solution of methyl 3-(hydroxymethyl)cyclobutanecarboxylate (CAS: 89941-55-9) (60.0 mg, 0.42 mmol), 6-(3-cyclopropyl-1,2,4-triazol-1-yl)-2-azaspiro[3.3]heptane (100 mg, 0.49 mmol) and 2,3,4,6,7,8-hexahydro-1H-pyrimido[1,2-a]pyrimidine (57.93 mg, 0.42 mmol) in THF (4.25 mL) was stirred at 75° C. for 16 h. The reaction mixture was concentrated in vacuo and the residue was purified by reverse flash (0.05% FA condition) then lyophilized to give the title compound (53.0 mg, 0.17 mmol, 40% yield) as a white solid. MS (ESI): m/z=317.2 [M+H]⁺

In analogy to Example 173, the following Examples were generated using the respective building blocks in Step a).

| | | | | MS, |
|---|---|---|---|---|
| | | | Building | ESI: |
| Ex. | Structure | Systematic Name | Blocks | m/z |
| 176 | 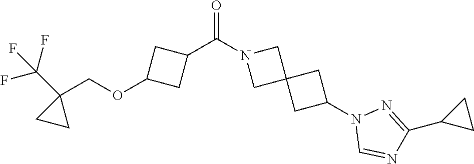 [SMILES: O=C(C1CC(OCC2(C(F)(F)F)CC2)C1)N1CC2(CC(n3cnc(C4CC4)n3)C2)C1] | [6-(3-cyclopropyl- 1,2,4-triazol-1-yl)-2- azaspiro[3.3]heptan- 2-yl]-[3-[[1- (trifluoromethyl) cyclopropyl]methoxy] cyclobutyl]methanone | methyl 3- hydroxycyclo- butanecarboxylate (CAS: 4934-99-0) | 425.2 [M + H]⁺ |

### Example 177

(6-(3-(azetidin-1-yl)-1H-1,2,4-triazol-1-yl)-2-azaspiro[3.3]heptan-2-yl)(3-((4-(trifluoromethyl)benzyl)oxy)azetidin-1-yl)methanone

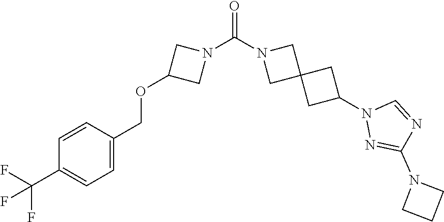 [SMILES: O=C(N1CC(OCc2ccc(C(F)(F)F)cc2)C1)N1CC2(CC(n3cnc(N4CCC4)n3)C2)C1]

To a solution of (6-(3-bromo-1H-1,2,4-triazol-1-yl)-2-azaspiro[3.3]heptan-2-yl)(3-((4-(trifluoromethyl)benzyl)oxy)azetidin-1-yl)methanone (80 mg, 160 μmol) in dry DMSO (1 mL) was added azetidine (45.6 mg, 799 μmol), copper (I) iodide (7.61 mg, 40 μmol), L-proline (4.6 mg, 40 μmol) and Cs₂CO₃ (104 mg, 320 μmol). The reaction mixture was then stirred at 90° C. for 18 h. Addition of copper (I) iodide (7.61 mg, 40 μmol), L-proline (4.6 mg, μmol), Cs₂CO₃ (104 mg, 320 μmol) and 300 mg of azetidine after which the reaction mixture was again stirred at 90° C. for 18 h. The insolubles were removed by filtration over a pad of celite, the filter pad was washed with DMSO and the crude filtrate was directly submitted for reversed-phase HPLC purification to yield of the title compound (17.5 mg). MS (ESI): m/z=477.3 [M+H]⁺

### Step a) (6-(3-bromo-]H-1,2,4-triazol-1-yl)-2-azaspiro[3.3]heptan-2-yl)(3-((4-(trifluoromethyl)benzyl)oxy)azetidin-1-yl)methanone

To a suspension of di(1H-1,2,4-triazol-1-yl)methanone (471 mg, 2.87 mmol) in dry CH₃CN (6 mL) cooled down to 0° C. under an inert atmosphere was slowly added a solution of 6-(3-bromo-1H-1,2,4-triazol-1-yl)-2-azaspiro[3.3]heptane (A.9) (665 mg, 2.74 mmol) and DIPEA (424 mg, 573 μL, 3.28 mmol) in dry CH₃CN (8 mL). The reaction was then stirred at 0° C. for 5 min and at RT for 1 h. Addition of 3-((4-(trifluoromethyl)benzyl)oxy)azetidine 4-methylbenzenesulfonate (B.20) (1.27 g, 3.15 mmol) and DIPEA (707 mg, 956 μL, 5.47 mmol) was then added and the reaction mixture was then stirred at 80° C. for 18 h. The reaction mixture was diluted with ethyl acetate and extracted with aq. Na₂CO₃ 1 M solution. The organic phase was collected and the aqueous phase was back-extracted with ethyl acetate. The combined organic phases were dried over sodium sulfate and evaporated down to dryness. The crude material was purified by flash chromatography with an eluent mixture of dichloromethane and methanol (0% to 10%) to yield the title compound (405 mg). MS (ESI): m/z=502.3 [M+H]⁺

### Example 178

### [6-(3-cyclopropyl-1,2,4-triazol-4-yl)-2-azaspiro[3.3]heptan-2-yl]−[rac-(3aS,6aR)-5-(2-chloro-4-fluoro-phenoxy)-3,3a,4,5,6,6a-hexahydro-1H-cyclopenta[c]pyrrol-2-yl]methanone

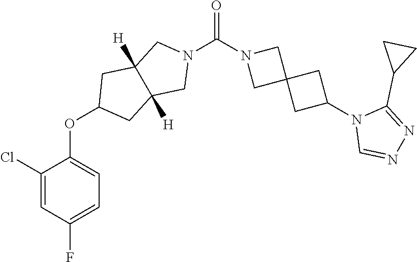 [SMILES: O=C(N1C[C@H]2CC(Oc3ccc(F)cc3Cl)C[C@H]2C1)N1CC2(CC(n3cnnc3C3CC3)C2)C1]

To a solution of rac-tert-butyl (3aR,6aS)-5-(2-chloro-4-fluorophenoxy)hexahydrocyclopenta[c]pyrrole-2(1H)-carboxylate (Step c) (28.9 mg, 81.2 μmol) in DCM (1 mL) was added TFA (92.6 mg, 62.6 μL, 812 μmol). The mixture was stirred at room temperature for 3 h. The solvent was evaporated under reduced pressure, TFA was coevaporated with toluene. The TFA salt was dissolved in acetonitrile (1 mL). The mixture was cooled to 0° C. DIPEA (21 mg, 28.4 μL, 162 μmol) and 4-nitrophenyl 6-(3-cyclopropyl-1H-1,2,4-triazol-1-yl)-2-azaspiro[3.3]heptane-2-carboxylate (Step b) (20 mg, 54.1 μmol) were added. The mixture was heated to 80° C. for 24 h. The solvent was removed under reduced pressure. The crude was purified by column chromatography using DCM/methanol (0-10% methanol) as solvent. The title compound (4 mg, 8.23 μmol, 15% yield) as obtained as a light red solid. MS (ESI): m/z=486.4 [M+H]⁺

### Step a) tert-butyl 6-(5-cyclopropyl-]H-1, 2,4-triazol-1-yl)-2-azaspiro[3.3]heptane-2-carboxylate

To a solution of tert-butyl 6-((methylsulfonyl)oxy)-2-azaspiro[3.3]heptane-2-carboxylate (2.05 g, 7.04 mmol) in DMF (61.1 mL) was added cesium carbonate (8.96 g, 27.5 mmol) followed by 3-cyclopropyl-1H-1,2,4-triazole (1.00 g, 9.16 mmol). The mixture was stirred at 100° C. for 24 h. The mixture was diluted with ethyl acetate and washed with water. The aqueous phase was extracted with ethyl acetate twice. The solvent was removed under reduced pressure and the crude was purified by HPLC. The title compound (600 mg, 1.97 mmol, 22% yield) was obtained as a white solid. (Note: side product obtained along with regioisomer.) MS (ESI): m/z=305.2 [M+H]⁺

### Step b) 4-nitrophenyl 6-(5-cyclopropyl-]H-1, 2,4-triazol-1-yl)-2-azaspiro[3.3]heptane-2-carboxylate

To a solution of tert-butyl 6-(5-cyclopropyl-1H-1,2,4-triazol-1-yl)-2-azaspiro[3.3]heptane-2-carboxylate (457 mg, 1.5 mmol) in DCM (12.2 mL) was added 2,2,2-trifluoroacetic acid (1.71 g, 1.15 mL, 15 mmol). The mixture was stirred at RTfor 5 h. The solvent was removed and TFA was coevaporated with toluene. The crude was redissolved in dry DCM (12.2 mL). The mixture was cooled to 0° C. TEA (760 mg, 1.05 mL, 7.51 mmol) was added followed by 4-nitrophenyl carbonochloridate (303 mg, 1.5 mmol). The mixture was allowed to warm up to RT and stirred for an additional 12 h. The solvent was removed under reduced pressure. The crude was purified by column chromatography using heptane/ethyl acetate (1:1) as solvent to yield the title compound (195 mg, 528 μmol, 35% yield) as a yellow solid. MS (ESI): m/z=370.1 [M+H]⁺

### Step c) rac-tert-butyl (3aR,6aS)-5-(2-chloro-4-fluorophenoxy)hexahydrocyclopenta[c]pyrrole-2(]H)-carboxylate

To a solution of 2-chloro-4-fluorophenol (135 mg, 100 μL, 921 μmol) in dry THF (4.61 mL) was added triphenylphosphine (266 mg, 1.01 mmol) followed by rac-tert-butyl (3aR,6aS)-5-hydroxyhexahydrocyclopenta[c]pyrrole-2(1H)-carboxylate (CAS: 875926-93-5) (209 mg, 921 μmol). The mixture was cooled to 0° C. DIAD (205 mg, 197 μL, 1.01 mmol) was added dropwise. The mixture was allowed to warm up to room temperature and stirred for 24 h. The reaction was stopped by addition of sat. aq. Na₂CO₃ solution (10 mL). The aqueous phase was extracted with DCM (3×20 mL). The organic phases were washed with aq. NaOH solution (30 mL, 1 M) and brine. The organic phases were dried over MgSO₄ and the solvent evaporated under reduced pressure. The crude was purified by column chromatography using heptane/ethyl acetate (0.30% EA) as solvent. The title compound (283 mg, 688 μmol, 75% yield) was obtained as a white solid. MS (ESI): m/z=300.2 [M-Boc+H]⁺

### Example 179

### [4-(5-tert-butyl-1,2,4-oxadiazol-3-yl)phenyl]−[6-(2-cyclopropyloxazol-5-yl)-6-hydroxy-2-azaspiro[3.3]heptan-2-yl]methanone

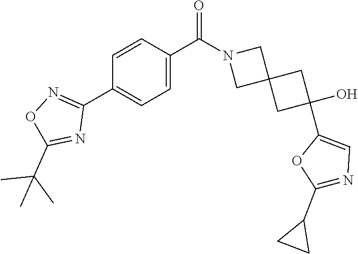 [SMILES: CC(C)(C)c1nc(-c2ccc(C(=O)N3CC4(C3)CC(O)(c3cnc(C5CC5)o3)C4)cc2)no1]

To a solution of 4-(5-(tert-butyl)-1,2,4-oxadiazol-3-yl)benzoic acid (99.5 mg, 404 μmol) in DMF (1 mL) was added DIPEA (348 mg, 470 μL, 2.69 mmol) followed by 2,4,6-tripropyl-1,3,5,2,4,6-trioxatriphosphinane 2,4,6-trioxide (685 mg, 489 μL, 1.08 mmol). 6-(2-cyclopropyloxazol-5-yl)-2-azaspiro[3.3]heptan-6-ol 2,2,2-trifluoroacetate (90 mg, 269 μmol) in DMF (1 mL) was added dropwise. The reaction was stirred at room temperature for 5 h. The mixture was diluted with ethyl acetate and washed with water. The aqueous phase was extracted twice with ethyl acetate. The combined organic phases were washed with water and brine and dried over MgSO₄. The solvent was evaporated under reduced pressure. The crude was purified by rpHPLC. The title compound (17 mg, 36.4 μmol, 14% yield) was obtained as a white solid. MS (ESI): m/z=449.3 [M+H]⁺

### Step a) tert-butyl 6-(2-cyclopropyloxazol-5-yl)-6-hydroxy-2-azaspiro[3.3]heptane-2-carboxylate

A solution of 2-cyclopropyloxazole (114 mg, 1.04 mmol) in dry THF(3.5 ml) was cooled down to 0° C. n-butyllithium (710 μL, 1.14 mmol, Eq: 1.2) was added dropwise over 10 minutes. The mixture was stirred for 30 minutes. tert-butyl 6-oxo-2-azaspiro[3.3]heptane-2-carboxylate (200 mg, 947 μmol) in dry THF (1.23 mL) was added dropwise. The mixture was stirred at 0° C. for 2 h. The mixture was poured onto ice. The mixture was extracted with ethyl acetate twice. The organic phases were washed with water and brine and dried over MgSO₄. The solvent was removed under reduced pressure. The crude was purified by column chromatography using ethyl acetate as solvent. The title compound (87 mg, 239 μmol, 25% yield) was obtained as a light brown oil. MS (ESI): m/z=321.3 [M+H]⁺

### Step b) 6-(2-cyclopropyloxazol-5-yl)-2-azaspiro[3.3]heptan-6-ol 2,2,2-trifluoroacetate

To a solution of tert-butyl 6-(2-cyclopropyloxazol-5-yl)-6-hydroxy-2-azaspiro[3.3]heptane-2-carboxylate (87 mg, 272 μmol) in DCM (1.36 mL) was added 2,2,2-trifluoroacetic acid (464 mg, 318 μL, 4.07 mmol). The mixture was stirred at room temperature for 4 h. The solvent was evaporated under reduced pressure, TFA was coevaporated with toluene. The title compound (90 mg, 240 μmol, 88% yield) was obtained as an off-white oil. MS (ESI): m/z=221.2 [M+H]⁺

### Example 181

### [3-(1-tert-butylpyrazol-4-yl)azetidin-1-yl]−[6-(3-cyclopropyl-1,2,4-triazol-1-yl)-2-azaspiro[3.3]heptan-2-yl]methanone

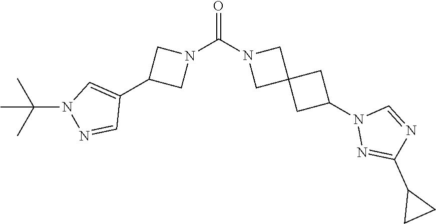 [SMILES: CC(C)(C)n1cc(C2CN(C(=O)N3CC4(CC(n5cnc(C6CC6)n5)C4)C3)C2)cn1]

To a solution of 4-bromo-1-(tert-butyl)-1H-pyrazole (98.3 mg, 0.484 mmol) in DME (5.0 mL) was added (6-(3-cyclopropyl-1H-1,2,4-triazol-1-yl)-2-azaspiro[3.3]heptan-2-yl)(3-iodoazetidin-1-yl)methanone (Example 33, Step b) (200 mg, 0.484 mmol), PMP (301 mg, 1.94 mmol) Ir[dF(CF₃)ppy]₂(dtbbpy)PF₆ (5.4 mg, 4.84 μmol), NiCl₂·DME (5.3 mg, 24.2 μmol), dtbbpy (6.5 mg, 24.2 μmol) and (TES)₃SiH (181 mg, 0.484 mmol) in a glove box. The mixture was stirred at room temperature (25° C.) for 48 h under 72 W blue LED strip irradiation. The mixture was filtered and washed with 1 mL of water. Then the mixture was extracted with EtOAc (2 mL×2). The organic layer was dried over anhydrous Na₂SO₄ and concentrated to give crude product. The crude product was purified by preparative HPLC to give the title compound (7.64 mg). MS (ESI): m/z=410.3 [M+H]⁺.

In analogy to Example 181, the Examples in the following table were generated, using the commercial bromide building blocks.

| | | | | MS, |
|---|---|---|---|---|
| | | | Building | ESI: |
| Ex. | Structure | Systematic Name | Blocks | m/z |
| 182 | 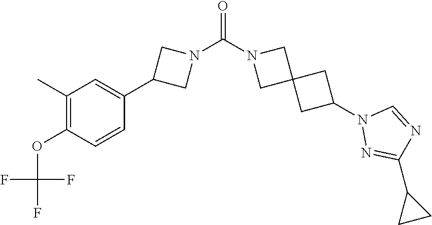 [SMILES: Cc1cc(C2CN(C(=O)N3CC4(CC(n5cnc(C6CC6)n5)C4)C3)C2)ccc1OC(F)(F)F] | [6-(3-cyclopropyl- 1,2,4-triazol-1-yl)-2- azaspiro[3.3]heptan-2- yl]-[3-[3-methyl-4- (trifluoromethoxy) phenyl]azetidin-1- yl]methanone | 4-bromo-2- methyl-1- (trifluoromethoxy) benzene (CAS: 887268- 26-0) | 462.3 [M + H]⁺ |

### Example 447

### [6-(3-cyclopropyl-1,2,4-triazol-1-yl)-2-azaspiro[3.3]heptan-2-yl]−[6-[[3-(trifluoromethylsulfonimidoyl)phenyl]methyl]-2-azaspiro[3.3]heptan-2-yl]methanone

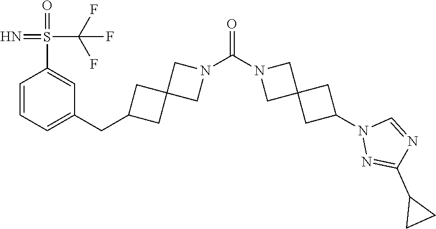 [SMILES: N=S(=O)(c1cccc(CC2CC3(C2)CN(C(=O)N2CC4(CC(n5cnc(C6CC6)n5)C4)C2)C3)c1)C(F)(F)F]

To a solution of 6-(3-cyclopropyl-1,2,4-triazol-1-yl)-2-azaspiro[3.3]heptane;2,2,2-trifluoroacetic acid (A.1) (40 mg, 0.126 mmol) in N,N-dimethylformamide (0.645 mL) cooled down to 0° C. was added DIPEA (154 μL, 0.880 mmol) followed by addition of bis(1,2,4-triazol-1-yl)methanone (21.7 mg, 0.132 mmol) after which the reaction mixture was stirred at 0° C. for 30 min. This was followed by addition of [3-(2-azaspiro[3.3]heptan-6-ylmethyl)phenyl]-imino-keto-(trifluoromethyl)-λ⁶-sulfane; tosylic acid (D.1) (64.7 mg, 0.132 mmol) to the reaction mixture, which was then stirred at 50° C. for 18 h. The crude reaction mixture was directly submitted for reversed-phase HPLC purification to yield 24.8 mg of the title compound as a white solid. MS (ESI): m/z=549.4 [M+H]⁺

In analogy to Example 1, Examples in the following table were generated, using the respective building blocks A.X and D.X. In some cases (marked with *) the reaction was carried out with isolated (6-(5-cyclopropyl-4H-1,2,4-triazol-3-yl)-2-axaspiro[3.3]heptan-2-yl]-(1,2,4-triazol-1-yl)methanone intermediate. In some cases, TEA can be used instead of DIPEA, and DMF and ACN can be used interchangeably as solvents.

| | | | | MS, |
|---|---|---|---|---|
| | | | Building | ESI: |
| Ex. | Structure | Systematic Name | Blocks | m/z |
| 446 | 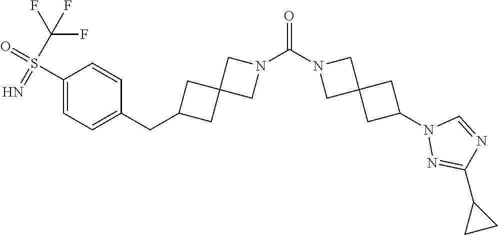 [SMILES: N=S(=O)(c1ccc(CC2CC3(C2)CN(C(=O)N2CC4(CC(n5cnc(C6CC6)n5)C4)C2)C3)cc1)C(F)(F)F] | [6-(3-cyclopropyl- 1,2,4-triazol-1-yl)-2- azaspiro[3.3]heptan-2- yl]-[6-[[4- (trifluoromethylsulfoni midoyl)phenyl]methyl]- 2-azaspiro[3.3]heptan- 2-yl]methanone | A.1 and D.2 | 549.4 [M + H]⁺ |
| 183* | 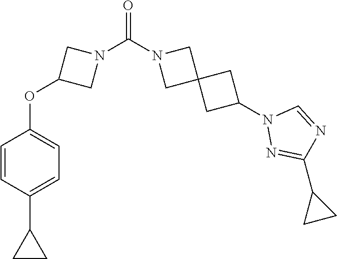 [SMILES: O=C(N1CC(Oc2ccc(C3CC3)cc2)C1)N1CC2(CC(n3cnc(C4CC4)n3)C2)C1] | (6-(3-cyclopropyl-1H- 1,2,4-triazol-1-yl)-2- azaspiro[3.3]heptan-2- yl)(3-(4- cyclopropylphenoxy) azetidin-1-yl)methanone | A.1 and D.228 | 420.3 [M + H]⁺ |
| 187* | 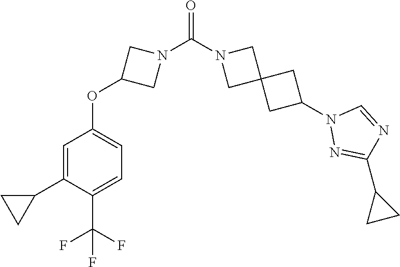 [SMILES: O=C(N1CC(Oc2ccc(C(F)(F)F)c(C3CC3)c2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[3-cyclopropyl-4- (trifluoromethyl)phenox y]azetidin-1- yl]methanone | A.1 and | 488.3 [M + H]⁺ |
| 188* | 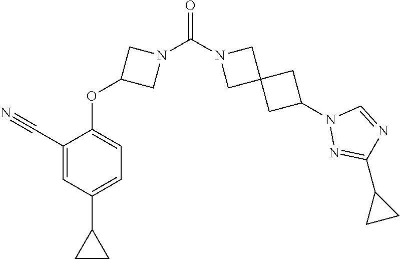 [SMILES: N#Cc1cc(C2CC2)ccc1OC1CN(C(=O)N2CC3(CC(n4cnc(C5CC5)n4)C3)C2)C1] | 5-cyclopropyl-2-[1-[6- (3-cyclopropyl-1,2,4- triazol-1-yl)-2- azaspiro[3.3]heptane-2- carbonyl]azetidin-3- yl]oxy-benzonitrile | A.1 and D.233 | 445.3 [M + H]⁺ |
| 189 | 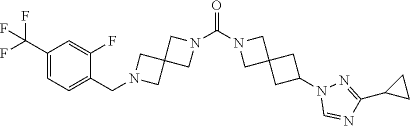 [SMILES: O=C(N1CC2(CC(n3cnc(C4CC4)n3)C2)C1)N1CC2(CN(Cc3ccc(C(F)(F)F)cc3F)C2)C1] | [6-(3-cyclopropyl- 1,2,4-triazol-1-yl)-2- azaspiro[3.3]heptan-2- yl]-[6-[[2-fluoro-4- (trifluoromethyl)phenyl] methyl]-2,6- diazaspiro[3.3]heptan- 2-yl]methanone | A.1 and D.303 | 505.4 [M + H]⁺ |
| 190* | 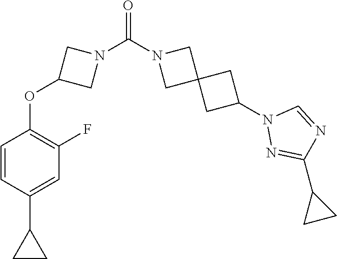 [SMILES: O=C(N1CC(Oc2ccc(C3CC3)cc2F)C1)N1CC2(CC(n3cnc(C4CC4)n3)C2)C1] | [3-(4-cyclopropyl-2- fluoro- phenoxy)azetidin-1-yl]- [6-(3-cyclopropyl- 1,2,4-triazol-1-yl)-2- azaspiro[3.3]heptan-2- yl]methanone | A.1 and D.229 | 438.3 [M + H]⁺ |
| 191* | 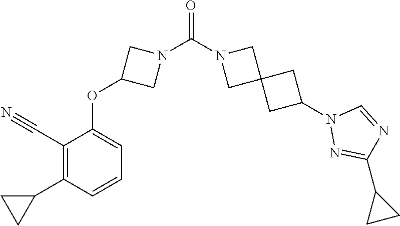 [SMILES: N#Cc1c(OC2CN(C(=O)N3CC4(CC(n5cnc(C6CC6)n5)C4)C3)C2)cccc1C1CC1] | 2-cyclopropyl-6-[1-[6- (3-cyclopropyl-1,2,4- triazol-1-yl)-2- azaspiro[3.3]heptane-2- carbonyl]azetidin-3- yl]oxy-benzonitrile | A.1 and D.226 | 445.3 [M + H]⁺ |
| 192* | 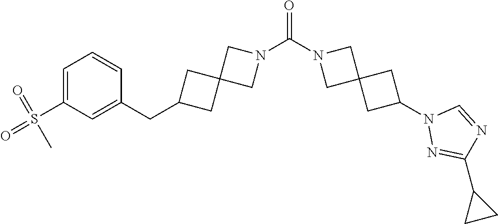 [SMILES: CS(=O)(=O)c1cccc(CC2CC3(C2)CN(C(=O)N2CC4(CC(n5cnc(C6CC6)n5)C4)C2)C3)c1] | [6-(3-cyclopropyl- 1,2,4-triazol-1-yl)-2- azaspiro[3.3]heptan-2- yl]-[6-(3-mesylbenzyl)- 2-azaspiro[3.3]heptan- 2-yl]methanone | A.1 and D.8 | 496.3 [M + H]⁺ |
| 193* | 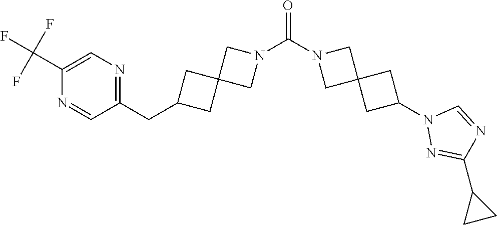 [SMILES: O=C(N1CC2(CC(Cc3cnc(C(F)(F)F)cn3)C2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[6-[[5- (trifluoromethyl)pyrazin- 2-yl]methyl]-2- azaspiro[3.3]heptan-2- yl]methanone | A.1 and D.4 | 488.3 [M + H]⁺ |
| 195 | 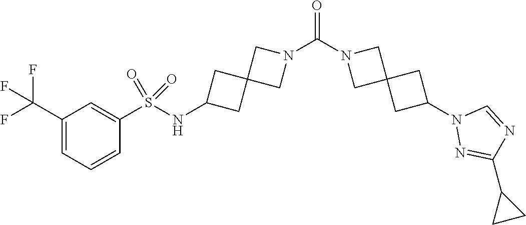 [SMILES: O=C(N1CC2(CC(NS(=O)(=O)c3cccc(C(F)(F)F)c3)C2)C1)N1CC2(CC(n3cnc(C4CC4)n3)C2)C1] | N-[2-[6-(3-cyclopropyl- 1,2,4-triazol-1-yl)-2- azaspiro[3.3]heptane-2- carbonyl]-2- azaspiro[3.3]heptan-6- yl]-3- (trifluoromethyl)benzen esulfonamide | A.1 and D.30 | 551.3 [M + H]⁺ |
| 196* | 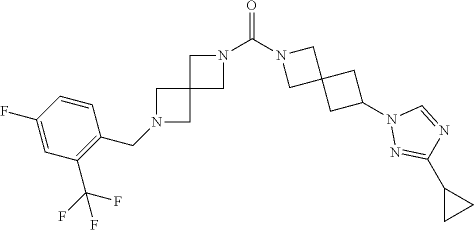 [SMILES: O=C(N1CC2(CC(n3cnc(C4CC4)n3)C2)C1)N1CC2(CN(Cc3ccc(F)cc3C(F)(F)F)C2)C1] | [6-(3-cyclopropyl- 1,2,4-triazol-1-yl)-2- azaspiro[3.3]heptan-2- yl]-[6-[[4-fluoro-2- (trifluoromethyl) phenyl]methyl]-2,6- diazaspiro[3.3]heptan- 2-yl]methanone | A.1 and D.110 | 505.4 [M + H]⁺ |
| 198* | 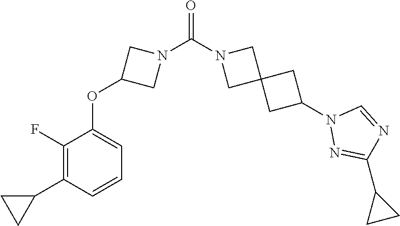 [SMILES: O=C(N1CC(Oc2cccc(C3CC3)c2F)C1)N1CC2(CC(n3cnc(C4CC4)n3)C2)C1] | [3-(3-cyclopropyl-2- fluoro- phenoxy)azetidin-1-yl]- [6-(3-cyclopropyl- 1,2,4-triazol-1-yl)-2- azaspiro[3.3]heptan-2- yl]methanone | A.1 and D.238 | 438.3 [M + H]⁺ |
| 199* | 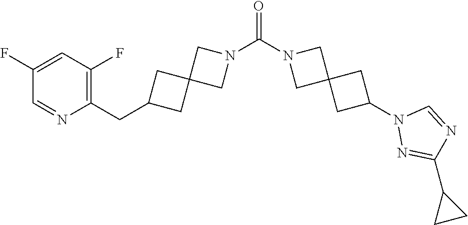 [SMILES: O=C(N1CC2(CC(Cc3ncc(F)cc3F)C2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[6-[(3,5-difluoro-2- pyridyl)methyl]-2- azaspiro[3.3]heptan-2- yl]methanone | A.1 and D.72 | 455.4 [M + H]⁺ |
| 200 | 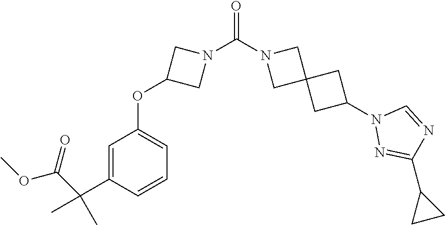 [SMILES: COC(=O)C(C)(C)c1cccc(OC2CN(C(=O)N3CC4(CC(n5cnc(C6CC6)n5)C4)C3)C2)c1] | methyl 2-[3-[1-[6-(3- cyclopropyl-1,2,4- triazol-1-yl)-2- azaspiro[3.3]heptane-2- carbonyl]azetidin-3- yl]oxyphenyl]-2- methyl-propanoate | A.1 and D.234 | 380.3 [M + H]⁺ |
| 201* | 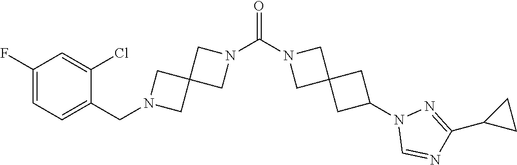 [SMILES: O=C(N1CC2(CC(n3cnc(C4CC4)n3)C2)C1)N1CC2(CN(Cc3ccc(F)cc3Cl)C2)C1] | (6-(2-chloro-4- fluorobenzyl)-2,6- diazaspiro[3.3]heptan- 2-yl)(6-(3-cyclopropyl- 1H-1,2,4-triazol-1-yl)- 2-azaspiro[3.3]heptan- 2-yl)methanone | A.1 and D.304 | 471.2 [M + H]⁺ |
| 202* | 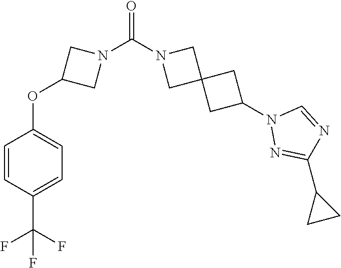 [SMILES: O=C(N1CC(Oc2ccc(C(F)(F)F)cc2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[4- (trifluoromethyl) phenoxy] azetidin-1- yl]methanone | A.1 and D.239 | 448.3 [M + H]⁺ |
| 205* | 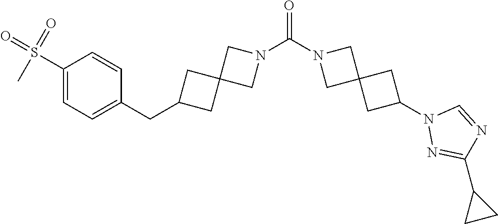 [SMILES: CS(=O)(=O)c1ccc(CC2CC3(C2)CN(C(=O)N2CC4(CC(n5cnc(C6CC6)n5)C4)C2)C3)cc1] | [6-(3-cyclopropyl- 1,2,4-triazol-1-yl)-2- azaspiro[3.3]heptan-2- yl]-[6-(4-mesylbenzyl)- 2-azaspiro[3.3]heptan- 2-yl]methanone | A.1 and D.7 | 496.3 [M + H]⁺ |
| 206 | 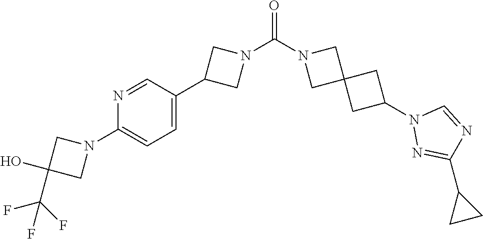 [SMILES: O=C(N1CC(c2ccc(N3CC(O)(C(F)(F)F)C3)nc2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[6-[3-hydroxy-3- (trifluoromethyl) azetidin-1-yl]-3- pyridyl]azetidin-1- yl]methanone | A.1 and D.196 | 504.3 [M + H]⁺ |
| 211* | 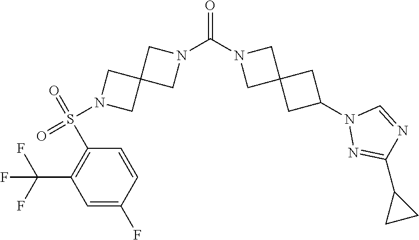 [SMILES: O=C(N1CC2(CC(n3cnc(C4CC4)n3)C2)C1)N1CC2(C1)CN(S(=O)(=O)c1ccc(F)cc1C(F)(F)F)C2] | (6-(3-cyclopropyl-1H- 1,2,4-triazol-1-yl)-2- azaspiro[3.3]heptan-2- yl)(6-((4-fluoro-2- (trifluoromethyl)phenyl) sulfonyl)-2,6- diazaspiro[3.3]heptan- 2-yl)methanone | A.1 and D.58 | 555.2 [M + H]⁺ |
| 212 | 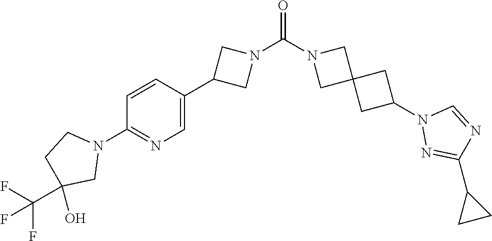 [SMILES: O=C(N1CC(c2ccc(N3CCC(O)(C(F)(F)F)C3)nc2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[6-[3-hydroxy-3- (trifluoromethyl) pyrrolidin-1-yl]-3- pyridyl]azetidin-1- yl]methanone | A.1 and D.189 | 518.3 [M + H]⁺ |
| 217* | 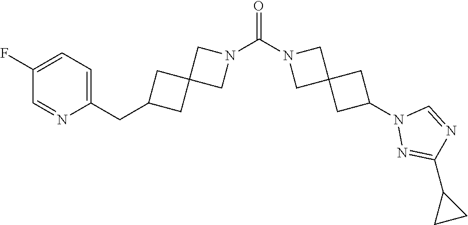 [SMILES: O=C(N1CC2(CC(Cc3ccc(F)cn3)C2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[6-[(5-fluoro-2- pyridyl)methyl]-2- azaspiro[3.3]heptan-2- yl]methanone | A.1 and D.22 | 437.4 [M + H]⁺ |
| 219 | 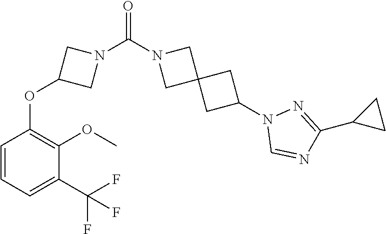 [SMILES: COc1c(OC2CN(C(=O)N3CC4(CC(n5cnc(C6CC6)n5)C4)C3)C2)cccc1C(F)(F)F] | (6-(3-cyclopropyl-1H- 1,2,4-triazol-1-yl)-2- azaspiro[3.3]heptan-2- yl)(3-(2-methoxy-3- (trifluoromethyl) phenoxy) azetidin-1- yl)methanone | A.1 and D.300 | 478.3 [M + H]⁺ |
| 221* | 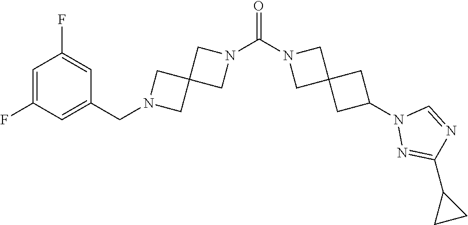 [SMILES: O=C(N1CC2(CC(n3cnc(C4CC4)n3)C2)C1)N1CC2(CN(Cc3cc(F)cc(F)c3)C2)C1] | [6-(3-cyclopropyl- 1,2,4-triazol-1-yl)-2- azaspiro[3.3]heptan-2- yl]-[6-[(3,5- difluorophenyl)methyl]- 2,6- diazaspiro[3.3]heptan- 2-yl]methanone | A.1 and D.100 | 455.4 [M + H]⁺ |
| 222 | 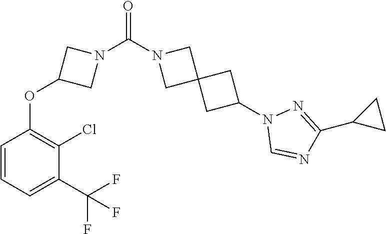 [SMILES: O=C(N1CC(Oc2cccc(C(F)(F)F)c2Cl)C1)N1CC2(CC(n3cnc(C4CC4)n3)C2)C1] | (3-(2-chloro-3- (trifluoromethyl) phenoxy) azetidin-1-yl)(6-(3- cyclopropyl-1H-1,2,4- triazol-1-yl)-2- azaspiro[3.3]heptan-2- yl)methanone | A.1 and D.299 | 482.2 [M + H]⁺ |
| 226* | 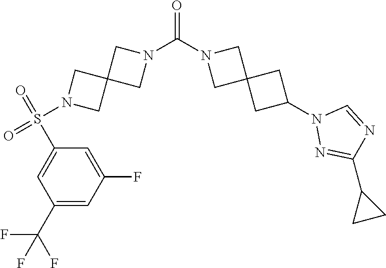 [SMILES: O=C(N1CC2(CC(n3cnc(C4CC4)n3)C2)C1)N1CC2(C1)CN(S(=O)(=O)c1cc(F)cc(C(F)(F)F)c1)C2] | [6-(3-cyclopropyl- 1,2,4-triazol-1-yl)-2- azaspiro[3.3]heptan-2- yl]-[2-[3-fluoro-5- (trifluoromethyl)phenyl] sulfonyl-2,6- diazaspiro[3.3]heptan- 6-yl]methanone | A.1 and D.62 | 555.4 [M + H]⁺ |
| 229* | 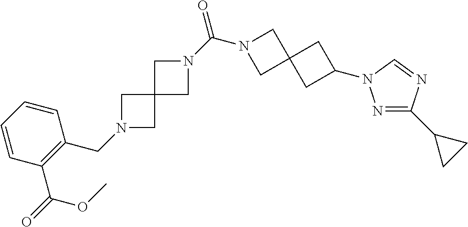 [SMILES: COC(=O)c1ccccc1CN1CC2(C1)CN(C(=O)N1CC3(CC(n4cnc(C5CC5)n4)C3)C1)C2] | methyl 2-[[2-[6-(3- cyclopropyl-1,2,4- triazol-1-yl)-2- azaspiro[3.3]heptane-2- carbonyl]-2,6- diazaspiro[3.3]heptan- 6-yl]methyl]benzoate | A.1 and D.112 | 477.3 [M + H]⁺ |
| 232* | 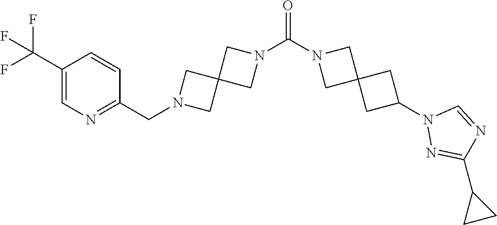 [SMILES: O=C(N1CC2(CC(n3cnc(C4CC4)n3)C2)C1)N1CC2(CN(Cc3ccc(C(F)(F)F)cn3)C2)C1] | [6-(3-cyclopropyl- 1,2,4-triazol-1-yl)-2- azaspiro[3.3]heptan-2- yl]-[6-[5- (trifluoromethyl)-2- pyridyl]methyl]-2,6- diazaspiro[3.3]heptan- 2-yl]methanone | A.1 and D.104 | 488.8 [M + H]⁺ |
| 234 | 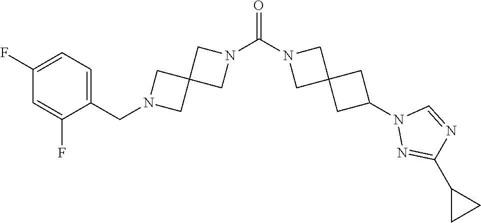 [SMILES: O=C(N1CC2(CC(n3cnc(C4CC4)n3)C2)C1)N1CC2(CN(Cc3ccc(F)cc3F)C2)C1] | [6-(3-cyclopropyl- 1,2,4-triazol-1-yl)-2- azaspiro[3.3]heptan-2- yl]-[6-[(2,4- difluorophenyl)methyl]- 2,6- diazaspiro[3.3]heptan- 2-yl]methanone | A.1 and D.99 | 455.4 [M + H]⁺ |
| 236* | 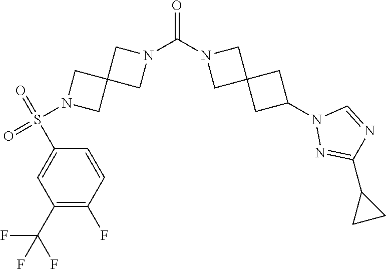 [SMILES: O=C(N1CC2(CC(n3cnc(C4CC4)n3)C2)C1)N1CC2(C1)CN(S(=O)(=O)c1ccc(F)c(C(F)(F)F)c1)C2] | [6-(3-cyclopropyl- 1,2,4-triazol-1-yl)-2- azaspiro[3.3]heptan-2- yl]-[2-[4-fluoro-3- (trifluoromethyl)phenyl] sulfonyl-2,6- diazaspiro[3.3]heptan- 6-yl]methanone | A.1 and D.60 | 555.4 [M + H]⁺ |
| 238 | 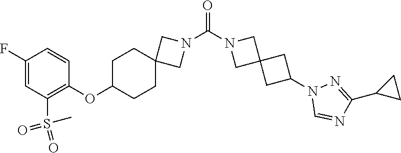 [SMILES: CS(=O)(=O)c1cc(F)ccc1OC1CCC2(CC1)CN(C(=O)N1CC3(CC(n4cnc(C5CC5)n4)C3)C1)C2] | [6-(3-cyclopropyl- 1,2,4-triazol-1-yl)-2- azaspiro[3.3]heptan-2- yl]-[7-(4-fluoro-2- mesyl-phenoxy)-2- azaspiro[3.5]nonan-2- yl]methanone | A.1 and D.269 | 544.3 [M + H]⁺ |
| 241 | 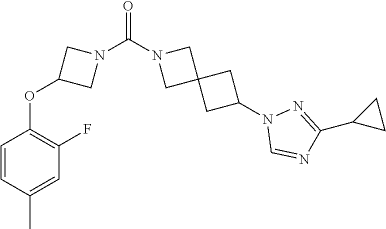 [SMILES: Cc1ccc(OC2CN(C(=O)N3CC4(CC(n5cnc(C6CC6)n5)C4)C3)C2)c(F)c1] | (6-(3-cyclopropyl-1H- 1,2,4-triazol-1-yl)-2- azaspiro[3.3]heptan-2- yl)(3-(2-fluoro-4- methylphenoxy)azetidin- 1-yl)methanone | A.1 and D.298 | 412.3 [M + H]⁺ |
| 247* | 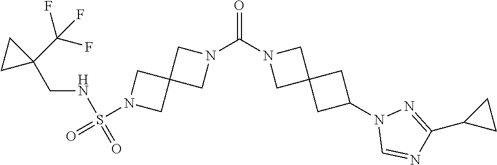 [SMILES: O=C(N1CC2(CC(n3cnc(C4CC4)n3)C2)C1)N1CC2(C1)CN(S(=O)(=O)NCC1(C(F)(F)F)CC1)C2] | 6-[6-(3-cyclopropyl- 1,2,4-triazol-1-yl)-2- azaspiro[3.3]heptane-2- carbonyl]-N-[[1- (trifluoromethyl) cyclopropyl] methyl]-2,6- diazaspiro[3.3]heptane- 2-sulfonamide | A.1 and D.152 | 530.3 [M + H]⁺ |
| 252 | 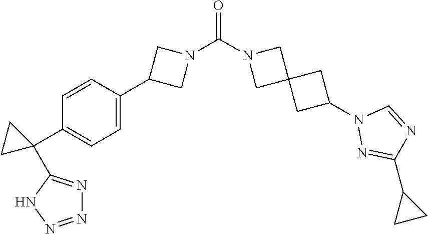 [SMILES: O=C(N1CC(c2ccc(C3(c4nnn[nH]4)CC3)cc2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[4-[1-(1H- tetrazol-5- yl)cyclopropyl]phenyl] azetidin-1-yl]methanone | A.1 and D.289 | 472.3 [M + H]⁺ |
| 253 | 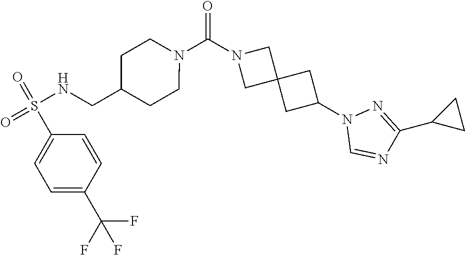 [SMILES: O=C(N1CCC(CNS(=O)(=O)c2ccc(C(F)(F)F)cc2)CC1)N1CC2(CC(n3cnc(C4CC4)n3)C2)C1] | N-[1-[6-(3- cyclopropyl-1,2,4- triazol-1-yl)-2- azaspiro[3.3]heptane-2- carbonyl]-4- piperidyl]methyl]-4- (trifluoromethyl) benzenesulfonamide | A.1 and D.176 | 553.4 [M + H]⁺ |
| 254* | 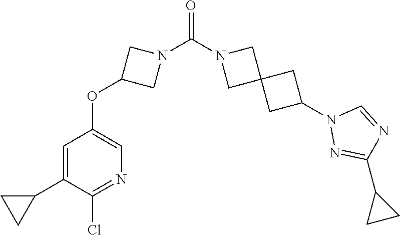 [SMILES: O=C(N1CC(Oc2cnc(Cl)c(C3CC3)c2)C1)N1CC2(CC(n3cnc(C4CC4)n3)C2)C1] | [3-[(6-chloro-5- cyclopropyl-3- pyridyl)oxy]azetidin-1- yl]-[6-(3-cyclopropyl- 1,2,4-triazol-1-yl)-2- azaspiro[3.3]heptan-2- yl]methanone | A.1 and D.243 | 455.3 [M + H]⁺ |
| 256* | 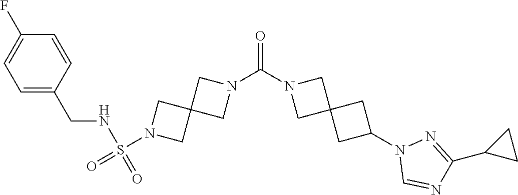 [SMILES: O=C(N1CC2(CC(n3cnc(C4CC4)n3)C2)C1)N1CC2(C1)CN(S(=O)(=O)NCc1ccc(F)cc1)C2] | 6-[6-(3-cyclopropyl- 1,2,4-triazol-1-yl)-2- azaspiro[3.3]heptane-2- carbonyl]-N-[(4- fluorophenyl)methyl]- 2,6- diazaspiro[3.3]heptane- 2-sulfonamide | A.1 and D.150 | 516.2 [M + H]⁺ |
| 257* | 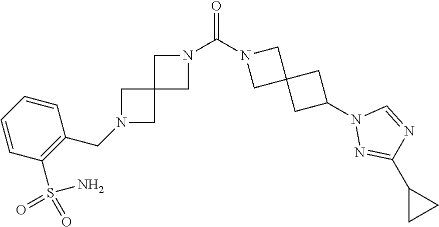 [SMILES: NS(=O)(=O)c1ccccc1CN1CC2(C1)CN(C(=O)N1CC3(CC(n4cnc(C5CC5)n4)C3)C1)C2] | 2-[[2-[6-(3- cyclopropyl-1,2,4- triazol-1-yl)-2- azaspiro[3.3]heptane-2- carbonyl]-2,6- diazaspiro[3.3]heptan- 6-yl]methyl] benzenesulfonamide | A.1 and D.111 | 498.4 [M + H]⁺ |
| 258* | 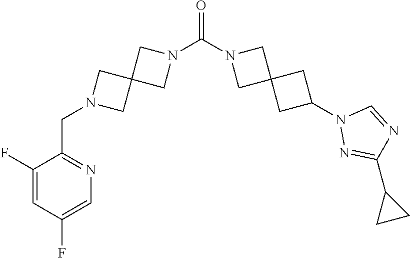 [SMILES: O=C(N1CC2(CC(n3cnc(C4CC4)n3)C2)C1)N1CC2(CN(Cc3ncc(F)cc3F)C2)C1] | [6-(3-cyclopropyl- 1,2,4-triazol-1-yl)-2- azaspiro[3.3]heptan-2- yl]-[6-[(3,5-difluoro-2- pyridyl)methyl]-2,6- diazaspiro[3.3]heptan- 2-yl]methanone | A.1 and D.106 | 456.4 [M + H]⁺ |
| 259 | 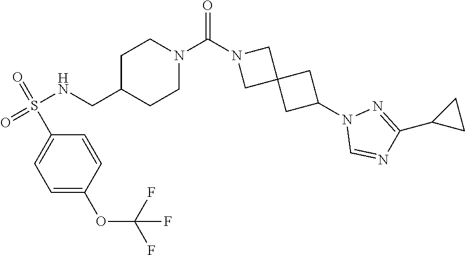 [SMILES: O=C(N1CCC(CNS(=O)(=O)c2ccc(OC(F)(F)F)cc2)CC1)N1CC2(CC(n3cnc(C4CC4)n3)C2)C1] | N-[[1-[6-(3- cyclopropyl-1,2,4- triazol-1-yl)-2- azaspiro[3.3]heptane-2- carbonyl]-4- piperidyl]methyl]-4- (trifluoromethoxy) benzenesulfonamide | A.1 and D.177 | 569.4 [M + H]⁺ |
| 260* | 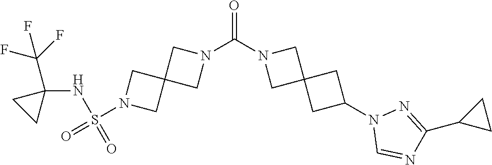 [SMILES: O=C(N1CC2(CC(n3cnc(C4CC4)n3)C2)C1)N1CC2(C1)CN(S(=O)(=O)NC1(C(F)(F)F)CC1)C2] | 6-[6-(3-cyclopropyl- 1,2,4-triazol-1-yl)-2- azaspiro[3.3]heptane-2- carbonyl]-N-[1- (trifluoromethyl) cyclopropyl]-2,6- diazaspiro[3.3]heptane- 2-sulfonamide | A.1 and D.151 | 516.2 [M + H]⁺ |
| 262 | 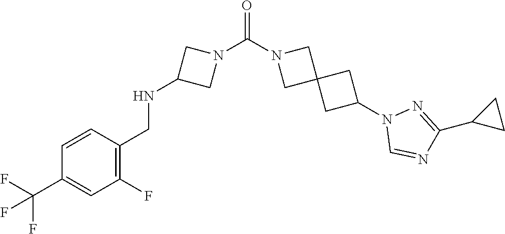 [SMILES: O=C(N1CC(NCc2ccc(C(F)(F)F)cc2F)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[[2-fluoro-4- (trifluoromethyl)phenyl] methylamino]azetidin- 1-yl]methanone | A.1 and D.132 | 479.1 [M + H]⁺ |
| 266 | 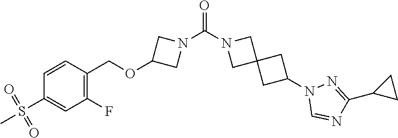 [SMILES: CS(=O)(=O)c1ccc(COC2CN(C(=O)N3CC4(CC(n5cnc(C6CC6)n5)C4)C3)C2)c(F)c1] | [6-(3-cyclopropyl- 1,2,4-triazol-1-yl)-2- azaspiro[3.3]heptan-2- yl]-[3-[(2-fluoro-4- methylsulfonyl- phenyl)methoxy] azetidin- 1-yl]methanone | A.1 and D.245 | 490.5 [M + H]⁺ |
| 267 | 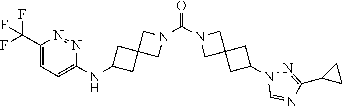 [SMILES: O=C(N1CC2(CC(Nc3ccc(C(F)(F)F)nn3)C2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[6-[[6- (trifluoromethyl) pyridazin- 3-yl]amino]-2- | A.1 and D.31 | 489.3 [M + H]⁺ |
| | | azaspiro[3.3]heptan-2- | | |
| | | yl]methanone | | |
| 268* | 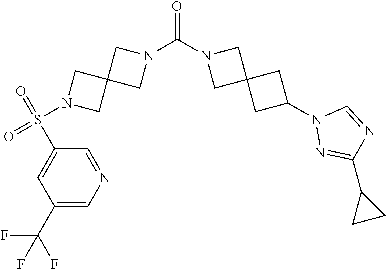 [SMILES: O=C(N1CC2(CC(n3cnc(C4CC4)n3)C2)C1)N1CC2(C1)CN(S(=O)(=O)c1cncc(C(F)(F)F)c1)C2] | [6-(3-cyclopropyl- 1,2,4-triazol-1-yl)-2- azaspiro[3.3]heptan-2- yl]-[2-[[5- (trifluoromethyl)-3- pyridyl]sulfonyl]-2,6- diazaspiro[3.3]heptan- 6-yl]methanone | A.1 and D.68 | 538.4 [M + H]⁺ |
| 269* | 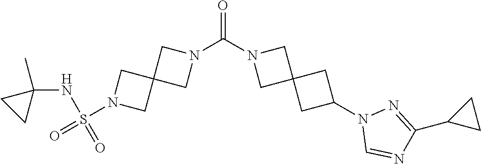 [SMILES: CC1(NS(=O)(=O)N2CC3(CN(C(=O)N4CC5(CC(n6cnc(C7CC7)n6)C5)C4)C3)C2)CC1] | 6-[6-(3-cyclopropyl- 1,2,4-triazol-1-yl)-2- azaspiro[3.3]heptane-2- carbonyl]-N-(1- methylcyclopropyl)- 2,6- diazaspiro[3.3]heptane- 2-sulfonamide | A.1 and D.149 | 462.3 [M + H]⁺ |
| 270 | 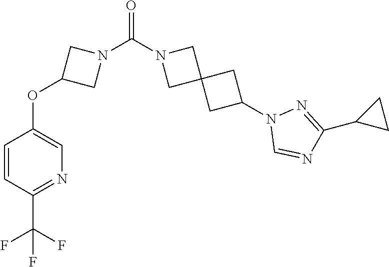 [SMILES: O=C(N1CC(Oc2ccc(C(F)(F)F)nc2)C1)N1CC2(CC(n3cnc(C4CC4)n3)C2)C1] | (6-(3-cyclopropyl-1H- 1,2,4-triazol-1-yl)-2- azaspiro[3.3]heptan-2- yl)(3-((6- (trifluoromethyl)pyridin- 3-yl)oxy)azetidin-1- yl)methanone | A.1 and D.297 | 449.2 [M + H]⁺ |
| 271* | 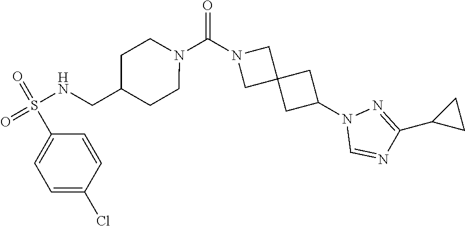 [SMILES: O=C(N1CCC(CNS(=O)(=O)c2ccc(Cl)cc2)CC1)N1CC2(CC(n3cnc(C4CC4)n3)C2)C1] | 4-chloro-N-[[1-[6-(3- cyclopropyl-1,2,4- triazol-1-yl)-2- azaspiro[3.3]heptane-2- carbonyl]-4- piperidyl]methyl benzenesulfonamide | A.1 and D.178 | 519.3 [M + H]⁺ |
| 273* | 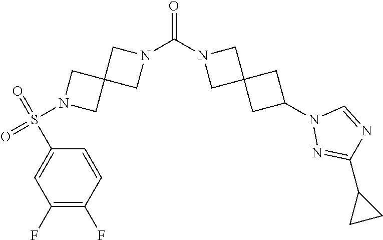 [SMILES: O=C(N1CC2(CC(n3cnc(C4CC4)n3)C2)C1)N1CC2(C1)CN(S(=O)(=O)c1ccc(F)c(F)c1)C2] | [6-(3-cyclopropyl- 1,2,4-triazol-1-yl)-2- azaspiro[3.3]heptan-2- yl]-[2-(3,4- difluorophenyl)sulfonyl- 2,6- diazaspiro[3.3]heptan- 6-yl]methanone | A.1 and D.65 | 505.4 [M + H]⁺ |
| 277* | 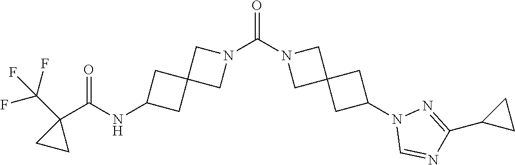 [SMILES: O=C(N1CC2(CC(NC(=O)C3(C(F)(F)F)CC3)C2)C1)N1CC2(CC(n3cnc(C4CC4)n3)C2)C1] | N-[2-[6-(3-cyclopropyl- 1,2,4-triazol-1-yl)-2- azaspiro[3.3]heptane-2- carbonyl]-2- azaspiro[3.3]heptan-6- yl]-1- (trifluoromethyl) cyclo- propanecarboxamide | A.1 and D.157 | 479.4 [M + H]⁺ |
| 305* | 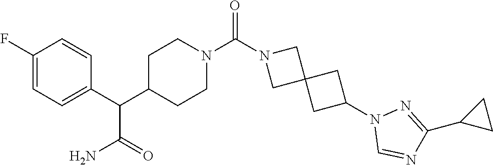 [SMILES: NC(=O)C(c1ccc(F)cc1)C1CCN(C(=O)N2CC3(CC(n4cnc(C5CC5)n4)C3)C2)CC1] | 2-[1-[6-(3-cyclopropyl- 1,2,4-triazol-1-yl)-2- azaspiro[3.3]heptane-2- carbonyl]-4-piperidyl]- 2-(4- fluorophenyl)acetamide | A.1 and D.275 | 467.5 [M + H]⁺ |
| 321 | 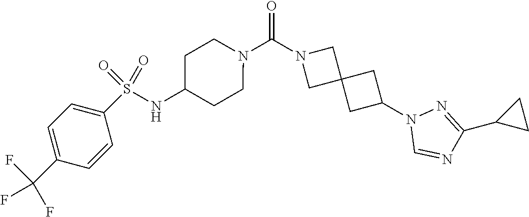 [SMILES: O=C(N1CCC(NS(=O)(=O)c2ccc(C(F)(F)F)cc2)CC1)N1CC2(CC(n3cnc(C4CC4)n3)C2)C1] | N-[1-[6-(3-cyclopropyl- 1,2,4-triazol-1-yl)-2- azaspiro[3.3]heptane-2- carbonyl]-4-piperidyl]- 4- (trifluoromethyl) benzenesulfonamide | A.1 and D.175 | 539.3 [M + H]⁺ |
| 341 | 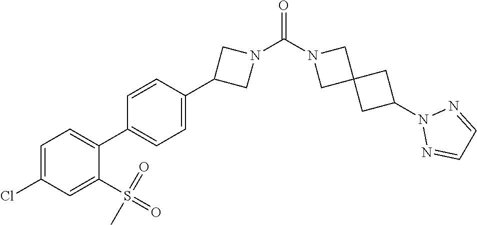 [SMILES: CS(=O)(=O)c1cc(Cl)ccc1-c1ccc(C2CN(C(=O)N3CC4(CC(n5nccn5)C4)C3)C2)cc1] | [3-[4-(4-chloro-2- methylsulfonyl- phenyl)phenyl]azetidin- 1-yl]-[6-(triazol-2-yl)- 2-azaspiro[3.3]heptan- 2-yl]methanone | A.14 and D.225 | 512.2 [M + H]⁺ |
| 342 | 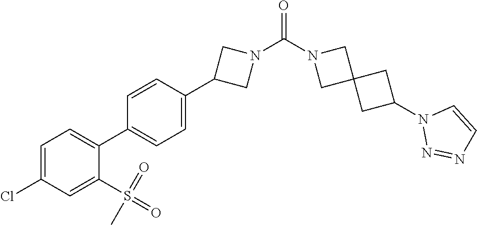 [SMILES: CS(=O)(=O)c1cc(Cl)ccc1-c1ccc(C2CN(C(=O)N3CC4(CC(n5ccnn5)C4)C3)C2)cc1] | [3-[4-(4-chloro-2- methylsulfonyl- phenyl)phenyl]azetidin- 1-yl]-[6-(triazol-1-yl)- 2-azaspiro[3.3]heptan- 2-yl]methanone | A.15 and D.225 | 512.2 [M + H]⁺ |
| 344 | 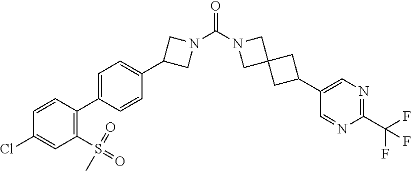 [SMILES: CS(=O)(=O)c1cc(Cl)ccc1-c1ccc(C2CN(C(=O)N3CC4(CC(c5cnc(C(F)(F)F)nc5)C4)C3)C2)cc1] | [3-[4-(4-chloro-2- methylsulfonyl- phenyl)phenyl]azetidin- 1-yl]-[6-[2- (trifluoromethyl) pyrimidin-5-yl]-2- azaspiro[3.3]heptan-2- yl]methanone | A.16 and D.225 | 591.1 [M + H]⁺ |
| 348 | 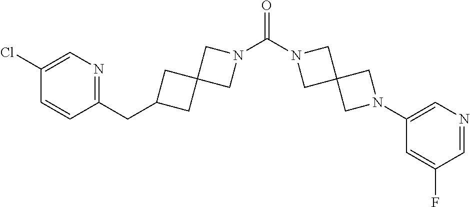 [SMILES: O=C(N1CC2(CC(Cc3ccc(Cl)cn3)C2)C1)N1CC2(C1)CN(c1cncc(F)c1)C2] | [6-[(5-chloro-2- pyridyl)methyl]-2- azaspiro[3.3]heptan-2- yl]-[6-(5-fluoro-3- pyridyl)-2,6- diazaspiro[3.3]heptan- 2-yl]methanone | A.18 and D.15 | 442.3 [M + H]⁺ |
| 349 | 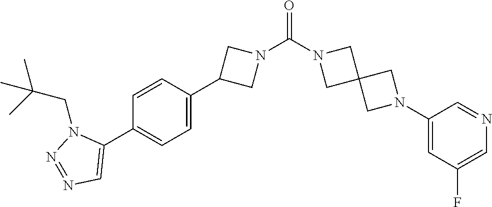 [SMILES: CC(C)(C)Cn1nncc1-c1ccc(C2CN(C(=O)N3CC4(C3)CN(c3cncc(F)c3)C4)C2)cc1] | [3-[4-[3-(2,2- dimethylpropyl)triazol- 4-yl]phenyl]azetidin-1- yl]-[6-(5-fluoro-3- pyridyl)-2,6- diazaspiro[3.3]heptan- 2-yl]methanone | A.18 and D.280 | 490.4 [M + H]⁺ |
| 350 | 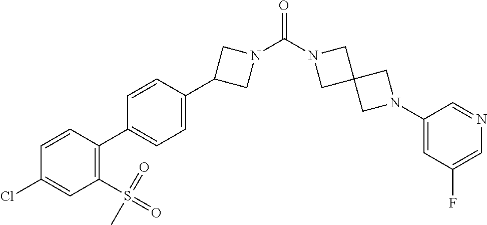 [SMILES: CS(=O)(=O)c1cc(Cl)ccc1-c1ccc(C2CN(C(=O)N3CC4(C3)CN(c3cncc(F)c3)C4)C2)cc1] | [3-[4-(4-chloro-2- methylsulfonyl- phenyl)phenyl]azetidin- 1-yl]-[6-(5-fluoro-3- pyridyl)-2,6- diazaspiro[3.3]heptan- 2-yl]methanone | A.18 and D.225 | 541.3 [M + H]⁺ |
| 351 | 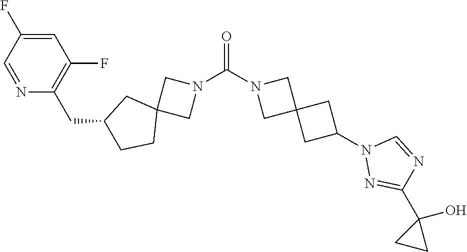 [SMILES: O=C(N1CC2(CC(n3cnc(C4(O)CC4)n3)C2)C1)N1CC2(CC[C@H](Cc3ncc(F)cc3F)C2)C1] | [6-[3-(1- hydroxycyclopropyl)- 1,2,4-triazol-1-yl]-2- azaspiro[3.3]heptan-2- yl]-[(6S)-6-[(3,5- difluoro-2- pyridyl)methyl]-2- azaspiro[3.4]octan-2- yl]methanone | A.10 and D.92 (chiral separatio n of racemic Ex. 388) | 485.4 [M + H]⁺ |
| | (enantiomer 2: arbitrary | | | |
| | assignment of stereochemistry) | | | |
| 352 | 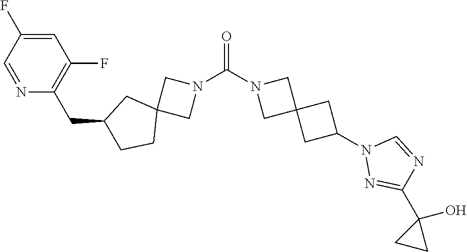 [SMILES: O=C(N1CC2(CC(n3cnc(C4(O)CC4)n3)C2)C1)N1CC2(CC[C@@H](Cc3ncc(F)cc3F)C2)C1] | [6-[3-(1- hydroxycyclopropyl)- 1,2,4-triazol-1-yl]-2- azaspiro[3.3]heptan-2- yl]-[(6R)-6-[(3,5- difluoro-2- pyridyl)methyl]-2- azaspiro[3.4]octan-2- yl]methanone | A.10 and D.92 (chiral separation of racemic Ex. 388) | 485.4 [M + H]⁺ |
| | (enantiomer 1: arbitrary | | | |
| | assignment of stereochemistry) | | | |
| 353 | 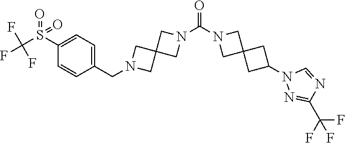 [SMILES: O=C(N1CC2(CC(n3cnc(C(F)(F)F)n3)C2)C1)N1CC2(CN(Cc3ccc(S(=O)(=O)C(F)(F)F)cc3)C2)C1] | [6-[[4- (trifluoromethylsulfonyl) phenyl]methyl]-2,6- diazaspiro[3.3]heptan- 2-yl]-[6-[3- (trifluoromethyl)-1,2,4- triazol-1-yl]-2- azaspiro[3.3]heptan-2- yl]methanone | A.13 and D.54 | 579.3 [M + H]⁺ |
| 355 | 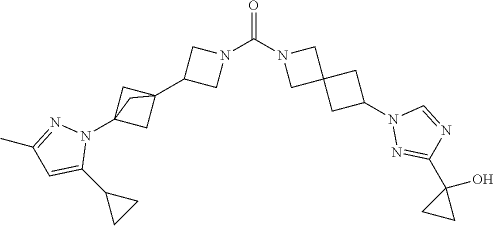 [SMILES: Cc1cc(C2CC2)n(C23CC(C4CN(C(=O)N5CC6(CC(n7cnc(C8(O)CC8)n7)C6)C5)C4)(C2)C3)n1] | [3-[3-(5-cyclopropyl-3- methyl-pyrazol-1-yl)-1- bicyclo[1.1.1]pentanyl] azetidin-1-yl]-[6-[3-(1- hydroxycyclopropyl)- 1,2,4-triazol-1-yl]-2- azaspiro[3.3]heptan-2- yl]methanone | A.10 and E.8 | 490.4 [M + H]⁺ |
| 358 | 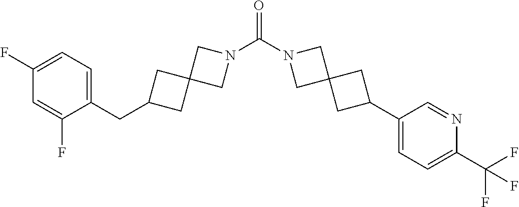 [SMILES: O=C(N1CC2(CC(Cc3ccc(F)cc3F)C2)C1)N1CC2(CC(c3ccc(C(F)(F)F)nc3)C2)C1] | [6-[(2,4- difluorophenyl)methyl]- 2-azaspiro[3.3]heptan- 2-yl]-[6-[6- (trifluoromethyl)-3- pyridyl]-2- azaspiro[3.3]heptan-2- yl]methanone | A.11 and B.14 | 492.2 [M + H]⁺ |
| 365 | 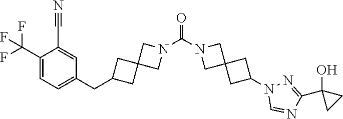 [SMILES: N#Cc1cc(CC2CC3(C2)CN(C(=O)N2CC4(CC(n5cnc(C6(O)CC6)n5)C4)C2)C3)ccc1C(F)(F)F] | 5-[[2-[6-[3-(1- hydroxycyclopropyl)- 1,2,4-triazol-1-yl]-2- azaspiro[3.3]heptane-2- carbonyl]-2- azaspiro[3.3]heptan-6- yl]methyl]-2- (trifluoromethyl) | A.10 and D.13 | 527.4 [M + H]⁺ |
| | | benzonitrile | | |
| 366 | 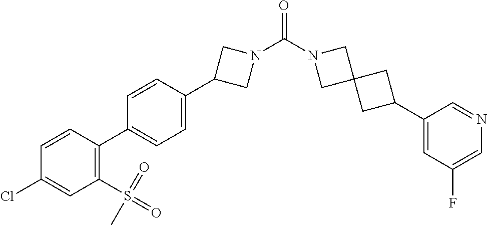 [SMILES: CS(=O)(=O)c1cc(Cl)ccc1-c1ccc(C2CN(C(=O)N3CC4(CC(c5cncc(F)c5)C4)C3)C2)cc1] | [3-[4-(4-chloro-2- methylsulfonyl- phenyl)phenyl]azetidin- 1-yl]-[6-(5-fluoro-3- pyridyl)-2- azaspiro[3.3]heptan-2- yl]methanone | A.12 and D.225 | 540.2 [M + H]⁺ |
| 367 | 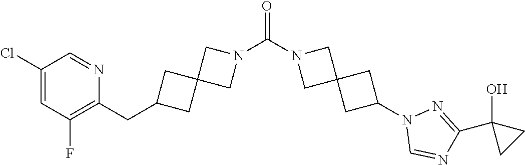 [SMILES: O=C(N1CC2(CC(Cc3ncc(Cl)cc3F)C2)C1)N1CC2(CC(n3cnc(C4(O)CC4)n3)C2)C1] | [6-[(5-chloro-3-fluoro- 2-pyridyl)methyl]-2- azaspiro[3.3]heptan-2- yl]-[6-[3-(1- hydroxycyclopropyl)- 1,2,4-triazol-1-yl]-2- azaspiro[3.3]heptan-2- yl]methanone | A.10 and D.305 | 487.3 [M + H]⁺ |
| 368 | 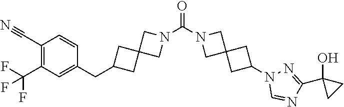 [SMILES: N#Cc1ccc(CC2CC3(C2)CN(C(=O)N2CC4(CC(n5cnc(C6(O)CC6)n5)C4)C2)C3)cc1C(F)(F)F] | 4-[[2-[6-[3-(1- hydroxycyclopropyl)- 1,2,4-triazol-1-yl]-2- azaspiro[3.3]heptane-2- carbonyl]-2- azaspiro[3.3]heptan-6- yl]methyl]-2- | A.10 and D.306 | 527.4 [M + H]⁺ |
| | | (trifluoromethyl) | | |
| | | benzonitrile | | |
| 370 | 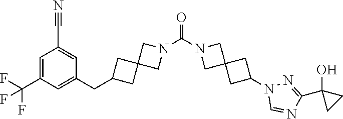 [SMILES: N#Cc1cc(CC2CC3(C2)CN(C(=O)N2CC4(CC(n5cnc(C6(O)CC6)n5)C4)C2)C3)cc(C(F)(F)F)c1] | 3-[[2-[6-[3-(1- hydroxycyclopropyl)- 1,2,4-triazol-1-yl]-2- azaspiro[3.3]heptane-2- carbonyl]-2- azaspiro[3.3]heptan-6- yl]methyl]-5- (trifluoromethyl) | A.10 and D.307 | 527.3 [M + H]⁺ |
| | | benzonitrile | | |
| 371 | 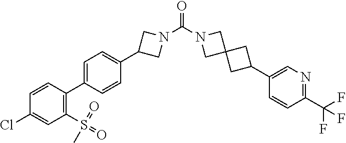 [SMILES: CS(=O)(=O)c1cc(Cl)ccc1-c1ccc(C2CN(C(=O)N3CC4(CC(c5ccc(C(F)(F)F)nc5)C4)C3)C2)cc1] | [3-[4-(4-chloro-2- methylsulfonyl- phenyl)phenyl]azetidin- 1-yl]-[6-[6- (trifluoromethyl)-3- pyridyl]-2- azaspiro[3.3]heptan-2- yl]methanone | A.11 and D.225 | 590.2 [M + H]⁺ |
| 372 | 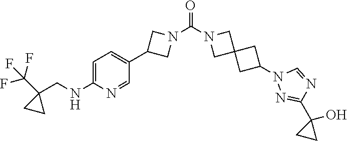 [SMILES: O=C(N1CC(c2ccc(NCC3(C(F)(F)F)CC3)nc2)C1)N1CC2(CC(n3cnc(C4(O)CC4)n3)C2)C1] | [6-[3-(1- hydroxycyclopropyl)- 1,2,4-triazol-1-yl]-2- azaspiro[3.3]heptan-2- yl]-[3-[6-[[1- (trifluoromethyl) cyclopropyl] methylamino]-3- pyridyl]azetidin-1- | A.10 and D.179 | 518.3 [M + H]⁺ |
| | | yl]methanone | | |
| 373 | 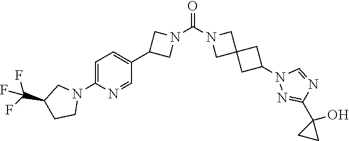 [SMILES: O=C(N1CC(c2ccc(N3CC[C@@H](C(F)(F)F)C3)nc2)C1)N1CC2(CC(n3cnc(C4(O)CC4)n3)C2)C1] | [6-[3-(1- hydroxycyclopropyl)- 1,2,4-triazol-1-yl]-2- azaspiro[3.3]heptan-2- yl]-[3-[6-[(3R)-3- (trifluoromethyl) pyrrolidin- 1-yl]-3-pyridyl] azetidin-1- | A.10 and D.188 | 518.3 [M + H]⁺ |
| | | yl]methanone | | |
| 374 | 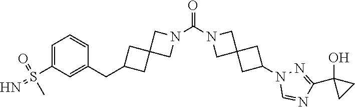 [SMILES: CS(=N)(=O)c1cccc(CC2CC3(C2)CN(C(=O)N2CC4(CC(n5cnc(C6(O)CC6)n5)C4)C2)C3)c1] | [6-[3-(1- hydroxycyclopropyl)- 1,2,4-triazol-1-yl]-2- azaspiro[3.3]heptan-2- yl]-[6-[[3- (methylsulfonimidoyl) phenyl]methyl]-2- | A.10 and D.79 | 511.4 [M + H]⁺ |
| | | azaspiro[3.3]heptan-2- | | |
| | | yl]methanone | | |
| 375 | 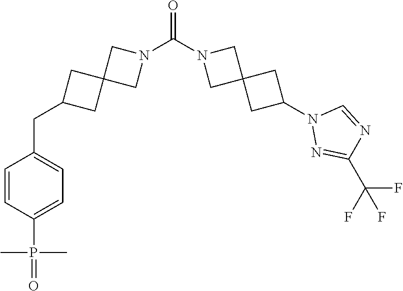 [SMILES: CP(C)(=O)c1ccc(CC2CC3(C2)CN(C(=O)N2CC4(CC(n5cnc(C(F)(F)F)n5)C4)C2)C3)cc1] | [6-[(4- dimethyl- phosphorylphenyl) methyl]-2- azaspiro[3.3]heptan-2- yl]-[6-[3- (trifluoromethyl)-1,2,4- triazol-1-yl]-2- azaspiro[3.3]heptan-2- yl]methanone | A.13 and E.1 | 522.3 [M + H]⁺ |
| 376 | 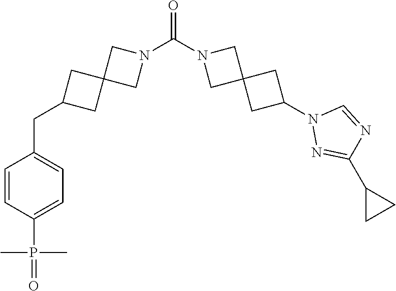 [SMILES: CP(C)(=O)c1ccc(CC2CC3(C2)CN(C(=O)N2CC4(CC(n5cnc(C6CC6)n5)C4)C2)C3)cc1] | [6-(3-cyclopropyl- 1,2,4-triazol-1-yl)-2- azaspiro[3.3]heptan-2- yl]-[6-[(4- dimethyl- phosphorylphenyl) methyl]-2- azaspiro[3.3]heptan-2- yl]methanone | A.1 and E.1 | 494.3 [M + H]⁺ |
| 377 | 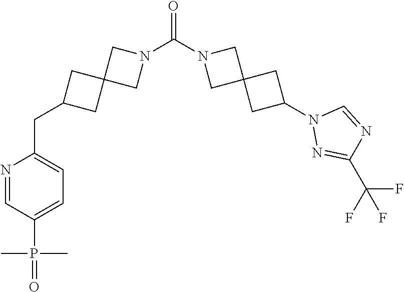 [SMILES: CP(C)(=O)c1ccc(CC2CC3(C2)CN(C(=O)N2CC4(CC(n5cnc(C(F)(F)F)n5)C4)C2)C3)nc1] | [6-[(5- dimethylphosphoryl-2- pyridyl)methyl]-2- azaspiro[3.3]heptan-2- yl]-[6-[3- (trifluoromethyl)-1,2,4- triazol-1-yl]-2- azaspiro[3.3]heptan-2- yl]methanone | A.13 and E.2 | 523.3 [M + H]⁺ |
| 378 | 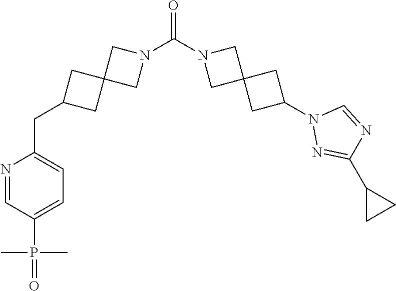 | [6-(3-cyclopropyl- 1,2,4-triazol-1-yl)-2- azaspiro[3.3]heptan-2- yl]-[6-[(5- dimethylphosphoryl-2- pyridyl)methyl]-2- azaspiro[3.3]heptan-2- yl]methanone | A.1 and E.2 | 539.4 [M + H]⁺ |
| 379 | 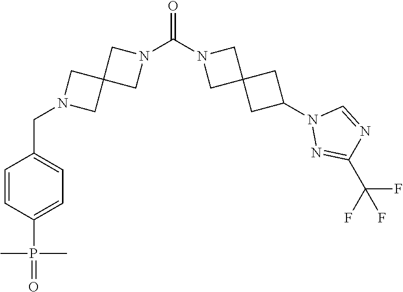 [SMILES: CP(C)(=O)c1ccc(CN2CC3(C2)CN(C(=O)N2CC4(CC(n5cnc(C(F)(F)F)n5)C4)C2)C3)cc1] | [6-[(4- dimethyl- phosphorylphenyl) methyl]-2,6- diazaspiro[3.3]heptan- 2-yl]-[6-[3- (trifluoromethyl)-1,2,4- triazol-1-yl]-2- azaspiro[3.3]heptan-2- yl]methanone | A.13 and E.3 | 523.3 [M + H]⁺ |
| 380 | 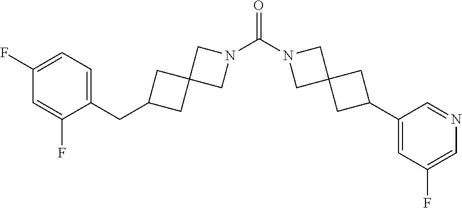 [SMILES: O=C(N1CC2(CC(Cc3ccc(F)cc3F)C2)C1)N1CC2(CC(c3cncc(F)c3)C2)C1] | [6-[(2,4- difluorophenyl)methyl]- 2-azaspiro[3.3]heptan- 2-yl]-[6-(5-fluoro-3- pyridyl)-2- azaspiro[3.3]heptan-2- yl]methanone | A.12 and B.14 | 442.3 [M + H]⁺ |
| 381 | 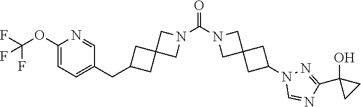 [SMILES: O=C(N1CC2(CC(Cc3ccc(OC(F)(F)F)nc3)C2)C1)N1CC2(CC(n3cnc(C4(O)CC4)n3)C2)C1] | [6-[3-(1- hydroxycyclopropyl)- 1,2,4-triazol-1-yl]-2- azaspiro[3.3]heptan-2- yl]-[6-[6- (trifluoromethoxy)-3- pyridyl]methyl]-2- | A.10 and D.302 | 519.4 [M + H]⁺ |
| | | azaspiro[3.3]heptan-2- | | |
| | | yl]methanone | | |
| 384 | 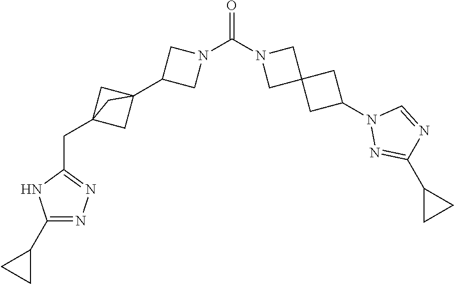 [SMILES: O=C(N1CC(C23CC(Cc4nnc(C5CC5)[nH]4)(C2)C3)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[3-[(5- cyclopropyl-4H-1,2,4- triazol-3-yl)methyl]-1- bicyclo[1.1.1]pentanyl] azetidin-1- yl]methanone | A.1 and E.7 | 475.4 [M + H]⁺ |
| 385 | 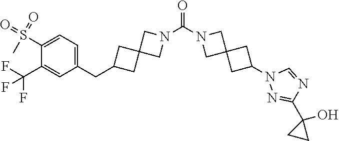 [SMILES: CS(=O)(=O)c1ccc(CC2CC3(C2)CN(C(=O)N2CC4(CC(n5cnc(C6(O)CC6)n5)C4)C2)C3)cc1C(F)(F)F] | [6-[3-(1- hydroxycyclopropyl)- 1,2,4-triazol-1-yl]-2- azaspiro[3.3]heptan-2- yl]-[6-[[4- methylsulfonyl-3- (trifluoromethyl)phenyl] methyl]-2- azaspiro[3.3]heptan-2- | A.10 and D.24 | 580.4 [M + H]⁺ |
| | | yl]methanone | | |
| 387 | 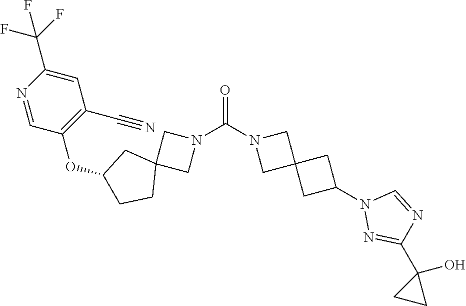 [SMILES: N#Cc1cc(C(F)(F)F)ncc1O[C@H]1CCC2(C1)CN(C(=O)N1CC3(CC(n4cnc(C5(O)CC5)n4)C3)C1)C2] | 5-[[(6S)-2-[6-[3-(1- hydroxycyclopropyl)- 1,2,4-triazol-1-yl]-2- azaspiro[3.3]heptane-2- carbonyl]-2- azaspiro[3.4]octan-6- yl]oxy]-2- (trifluoromethyl)pyridine- 4-carbonitrile | A.10 and D.97 | 544.4 [M + H]⁺ |
| 388 | 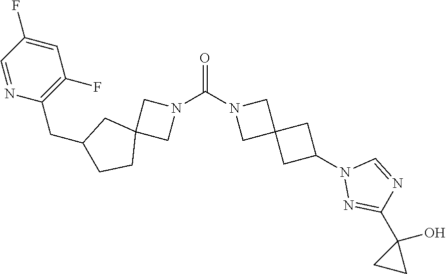 [SMILES: O=C(N1CC2(CCC(Cc3ncc(F)cc3F)C2)C1)N1CC2(CC(n3cnc(C4(O)CC4)n3)C2)C1] | [6-[(3,5-difluoro-2- pyridyl)methyl]-2- azaspiro[3.4]octan-2- yl]-[6-[3-(1- hydroxycyclopropyl)- 1,2,4-triazol-1-yl]-2- azaspiro[3.3]heptan-2- yl]methanone | A.10 and D.92 | 485.4 [M + H]⁺ |
| 389 | 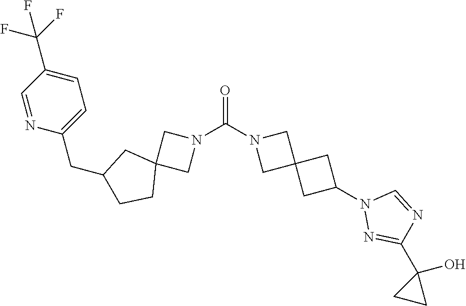 [SMILES: O=C(N1CC2(CCC(Cc3ccc(C(F)(F)F)cn3)C2)C1)N1CC2(CC(n3cnc(C4(O)CC4)n3)C2)C1] | [6-[3-(1- hydroxycyclopropyl)- 1,2,4-triazol-1-yl]-2- azaspiro[3.3]heptan-2- yl]-[6-[[5- (trifluoromethyl)-2- pyridyl]methyl]-2- azaspiro[3.4]octan-2- yl]methanone | A.10 and D.26 | 517.4 [M + H]⁺ |
| 390 | 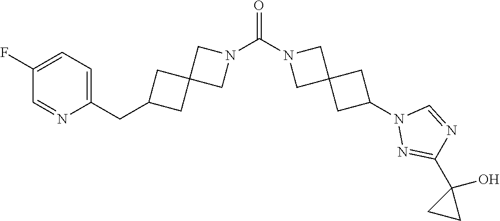 [SMILES: O=C(N1CC2(CC(Cc3ccc(F)cn3)C2)C1)N1CC2(CC(n3cnc(C4(O)CC4)n3)C2)C1] | [6-[(5-fluoro-2- pyridyl)methyl]-2- azaspiro[3.3]heptan-2- yl]-[6-[3-(1- hydroxycyclopropyl)- 1,2,4-triazol-1-yl]-2- azaspiro[3.3]heptan-2- yl]methanone | A.10 and D.22 | 453.4 [M + H]⁺ |
| 391 | 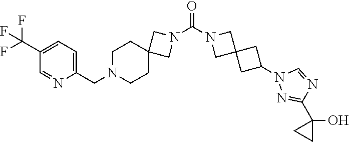 [SMILES: O=C(N1CC2(CCN(Cc3ccc(C(F)(F)F)cn3)CC2)C1)N1CC2(CC(n3cnc(C4(O)CC4)n3)C2)C1] | [6-[3-(1- hydroxycyclopropyl)- 1,2,4-triazol-1-yl]-2- azaspiro[3.3]heptan-2- yl]-[7-[[5- (trifluoromethyl)-2- pyridyl]methyl]-2,7- diazaspiro[3.5]nonan-2- yl]methanone | A.10 and D.118 | 532.4 [M + H]⁺ |
| 392 | 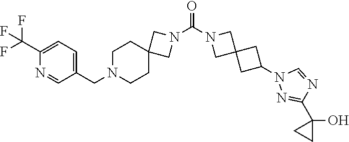 [SMILES: O=C(N1CC2(CCN(Cc3ccc(C(F)(F)F)nc3)CC2)C1)N1CC2(CC(n3cnc(C4(O)CC4)n3)C2)C1] | [6-[3-(1- hydroxycyclopropyl)- 1,2,4-triazol-1-yl]-2- azaspiro[3.3]heptan-2- yl]-[7-[[6- (trifluoromethyl)-3- pyridyl]methyl]-2,7- diazaspiro[3.5]nonan-2- yl]methanone | A.10 and D.116 | 532.5 [M + H]⁺ |
| 393 | 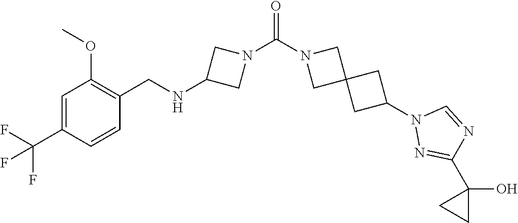 [SMILES: COc1cc(C(F)(F)F)ccc1CNC1CN(C(=O)N2CC3(CC(n4cnc(C5(O)CC5)n4)C3)C2)C1] | [6-[3-(1- hydroxycyclopropyl)- 1,2,4-triazol-1-yl]-2- azaspiro[3.3]heptan-2- yl]-[3-[[2-methoxy-4- (trifluoromethyl)phenyl] methylamino]azetidin- 1-yl]methanone | A.10 and D.137 | 507.4 [M + H]⁺ |
| 395 | 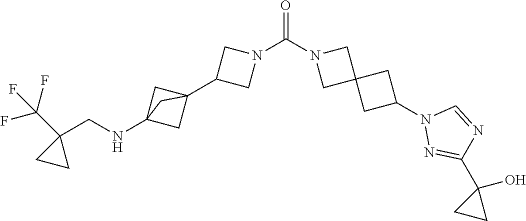 [SMILES: O=C(N1CC(C23CC(NCC4(C(F)(F)F)CC4)(C2)C3)C1)N1CC2(CC(n3cnc(C4(O)CC4)n3)C2)C1] | [6-[3-(1- hydroxycyclopropyl)- 1,2,4-triazol-1-yl]-2- azaspiro[3.3]heptan-2- yl]-[3-[3-[[1- (trifluoromethyl) cyclopropyl] methylamino]-1- bicyclo[1.1.1]pentanyl] azetidin-1- yl]methanone | A.10 and D.256 | 507.5 [M + H]⁺ |
| 396 | 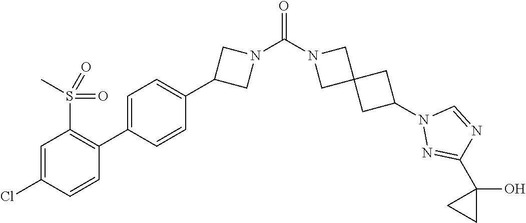 [SMILES: CS(=O)(=O)c1cc(Cl)ccc1-c1ccc(C2CN(C(=O)N3CC4(CC(n5cnc(C6(O)CC6)n5)C4)C3)C2)cc1] | [3-[4-(4-chloro-2- methylsulfonyl- phenyl)phenyl]azetidin- 1-yl]-[6-[3-(1- hydroxycyclopropyl)- 1,2,4-triazol-1-yl]-2- azaspiro[3.3]heptan-2- yl]methanone | A.10 and D.225 | 568.4 [M + H]⁺ |
| 397 | 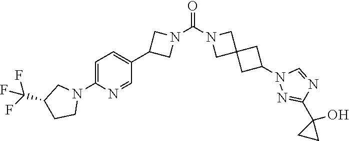 [SMILES: O=C(N1CC(c2ccc(N3CC[C@H](C(F)(F)F)C3)nc2)C1)N1CC2(CC(n3cnc(C4(O)CC4)n3)C2)C1] | [6-[3-(1- hydroxycyclopropyl)- 1,2,4-triazol-1-yl]-2- azaspiro[3.3]heptan-2- yl]-[3-[6-[(3S)-3- (trifluoromethyl) pyrrolidin-1-yl]-3- pyridyl]azetidin-1- yl]methanone | A.10 and D.187 | 518.4 [M + H]⁺ |
| 398 | 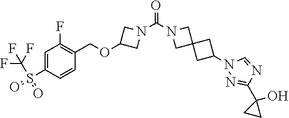 [SMILES: O=C(N1CC(OCc2ccc(S(=O)(=O)C(F)(F)F)cc2F)C1)N1CC2(CC(n3cnc(C4(O)CC4)n3)C2)C1] | [3-[[2-fluoro-4- (trifluoromethylsulfonyl) phenyl]methoxy]azetidin- 1-yl]-[6-[3-(1- hydroxycyclopropyl)- 1,2,4-triazol-1-yl]-2- azaspiro[3.3]heptan-2- yl]methanone | A.10 and D.225 | 560.4 [M + H]⁺ |
| 399 | 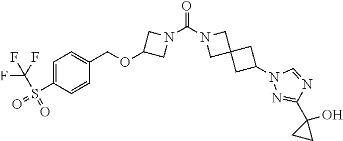 [SMILES: O=C(N1CC(OCc2ccc(S(=O)(=O)C(F)(F)F)cc2)C1)N1CC2(CC(n3cnc(C4(O)CC4)n3)C2)C1] | [6-[3-(1- hydroxycyclopropyl)- 1,2,4-triazol-1-yl]-2- azaspiro[3.3]heptan-2- yl]-[3-[[4- (trifluoromethylsulfonyl) phenyl]methoxy]azetidin- 1-yl]methanone | A.10 and D.232 | 542.3 [M + H]⁺ |
| 400 | 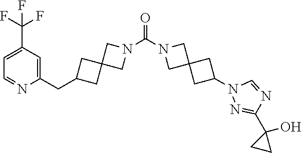 [SMILES: O=C(N1CC2(CC(Cc3cc(C(F)(F)F)ccn3)C2)C1)N1CC2(CC(n3cnc(C4(O)CC4)n3)C2)C1] | [6-[3-(1- hydroxycyclopropyl)- 1,2,4-triazol-1-yl]-2- azaspiro[3.3]heptan-2- yl]-[6-[4- (trifluoromethyl)-2- pyridyl]methyl]-2- azaspiro[3.3]heptan-2- yl]methanone | A.10 and D.74 | 517.4 [M + H]⁺ |
| 401 | 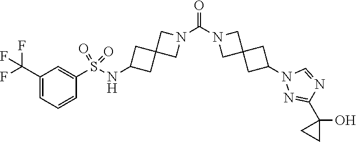 [SMILES: O=C(N1CC2(CC(NS(=O)(=O)c3cccc(C(F)(F)F)c3)C2)C1)N1CC2(CC(n3cnc(C4(O)CC4)n3)C2)C1] | N-[2-[6-[3-(1- hydroxycyclopropyl)- 1,2,4-triazol-1-yl]-2- azaspiro[3.3]heptane-2- carbonyl]-2- azaspiro[3.3]heptan-6- yl]-3- (trifluoromethyl) benzenesulfonamide | A.10 and D.30 | 551.3 [M + H]⁺ |
| 402 | 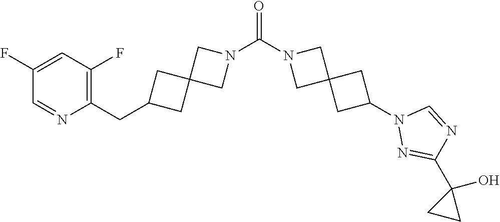 [SMILES: O=C(N1CC2(CC(Cc3ncc(F)cc3F)C2)C1)N1CC2(CC(n3cnc(C4(O)CC4)n3)C2)C1] | [6-[(3,5-difluoro-2- pyridyl)methyl]-2- azaspiro[3.3]heptan-2- yl]-[6-[3-(1- hydroxycyclopropyl)- 1,2,4-triazol-1-yl]-2- azaspiro[3.3]heptan-2- yl]methanone | A.10 and D.72 | 471.4 [M + H]⁺ |
| 403 | 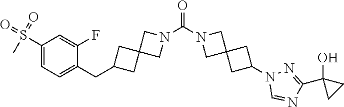 [SMILES: CS(=O)(=O)c1ccc(CC2CC3(C2)CN(C(=O)N2CC4(CC(n5cnc(C6(O)CC6)n5)C4)C2)C3)c(F)c1] | [6-[(2-fluoro-4- methylsulfonyl- phenyl)methyl]-2- azaspiro[3.3]heptan-2- yl]-[6-[3-(1- hydroxycyclopropyl)- 1,2,4-triazol-1-yl]-2- | A.10 and D.82 | 530.4 [M + H]⁺ |
| | | azaspiro[3.3]heptan-2- | | |
| | | yl]methanone | | |
| 404 | 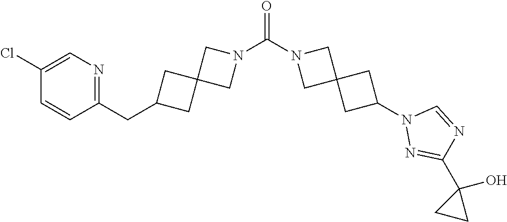 [SMILES: O=C(N1CC2(CC(Cc3ccc(Cl)cn3)C2)C1)N1CC2(CC(n3cnc(C4(O)CC4)n3)C2)C1] | [6-[(5-chloro-2- pyridyl)methyl]-2- azaspiro[3.3]heptan-2- yl]-[6-[3-(1- hydroxycyclopropyl)- 1,2,4-triazol-1-yl]-2- azaspiro[3.3]heptan-2- yl]methanone | A.10 and D.15 | 469.4 [M + H]⁺ |
| 405 | 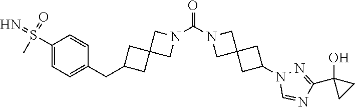 [SMILES: CS(=N)(=O)c1ccc(CC2CC3(C2)CN(C(=O)N2CC4(CC(n5cnc(C6(O)CC6)n5)C4)C2)C3)cc1] | [6-[3-(1- hydroxycyclopropyl)- 1,2,4-triazol-1-yl]-2- azaspiro[3.3]heptan-2- yl]-[6-[[4- (methylsulfonimidoyl) phenyl]methyl]-2- | A.10 and D.78 | 511.4 [M + H]⁺ |
| | | azaspiro[3.3]heptan-2- | | |
| | | yl]methanone | | |
| 406 | 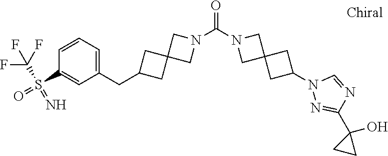 [SMILES: N=[S@@](=O)(c1cccc(CC2CC3(C2)CN(C(=O)N2CC4(CC(n5cnc(C6(O)CC6)n5)C4)C2)C3)c1)C(F)(F)F] | [6-[3-(1- hydroxycyclopropyl)- 1,2,4-triazol-1-yl]-2- azaspiro[3.3]heptan-2- yl]-[6-[[3- (trifluoromethyl- sulfonimidoyl) phenyl]methyl]- 2-azaspiro[3.3]heptan- 2-yl]methanone | A.10 and D.80b | 565.4 [M + H]⁺ |
| | (arbitrary assignment of | | | |
| | stereochemistry) | | | |
| 407 | 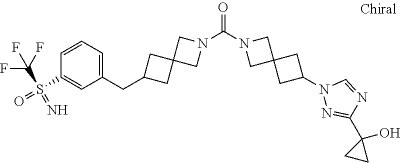 [SMILES: N=[S@](=O)(c1cccc(CC2CC3(C2)CN(C(=O)N2CC4(CC(n5cnc(C6(O)CC6)n5)C4)C2)C3)c1)C(F)(F)F] | [6-[3-(1- hydroxycyclopropyl)- 1,2,4-triazol-1-yl]-2- azaspiro[3.3]heptan-2- yl]-[6-[[3- (trifluoromethyl- sulfonimidoyl) phenyl]methyl]- 2-azaspiro[3.3]heptan- 2-yl]methanone | A.10 and D.80a | 565.4 [M + H]⁺ |
| | (arbitrary assignment of | | | |
| | stereochemistry) | | | |
| 408 | 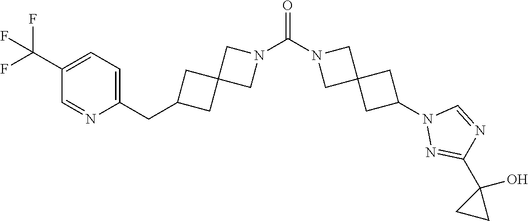 [SMILES: O=C(N1CC2(CC(Cc3ccc(C(F)(F)F)cn3)C2)C1)N1CC2(CC(n3cnc(C4(O)CC4)n3)C2)C1] | [6-[3-(1- hydroxycyclopropyl)- 1,2,4-triazol-1-yl]-2- azaspiro[3.3]heptan-2- yl]-[6-[[5- (trifluoromethyl)-2- pyridyl]methyl]-2- azaspiro[3.3]heptan-2- yl]methanone | A.10 and D.69 | 503.4 [M + H]⁺ |
| 409 | 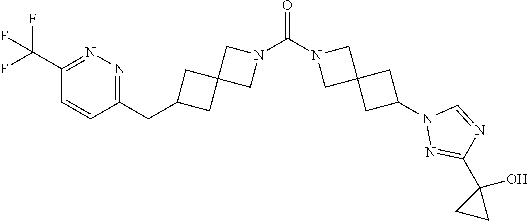 [SMILES: O=C(N1CC2(CC(Cc3ccc(C(F)(F)F)nn3)C2)C1)N1CC2(CC(n3cnc(C4(O)CC4)n3)C2)C1] | [6-[3-(1- hydroxycyclopropyl)- 1,2,4-triazol-1-yl]-2- azaspiro[3.3]heptan-2- yl]-[6-[[6- (trifluoromethyl) pyridazin- 3-yl]methyl]-2- azaspiro[3.3]heptan-2- yl]methanone | A.10 and D.11 | 504.4 [M + H]⁺ |
| 410 | 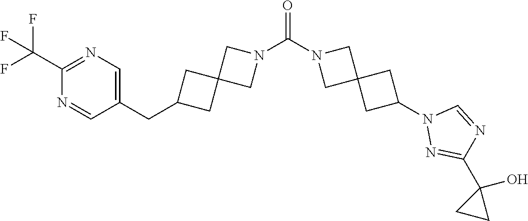 [SMILES: O=C(N1CC2(CC(Cc3cnc(C(F)(F)F)nc3)C2)C1)N1CC2(CC(n3cnc(C4(O)CC4)n3)C2)C1] | [6-[3-(1- hydroxycyclopropyl)- 1,2,4-triazol-1-yl]-2- azaspiro[3.3]heptan-2- yl]-[6-[[2- (trifluoromethyl) pyrimidin- 5-yl]methyl]-2- azaspiro[3.3]heptan-2- yl]methanone | A.10 and D.10 | 504.4 [M + H]⁺ |
| 411 | 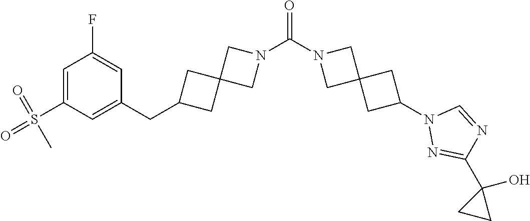 [SMILES: CS(=O)(=O)c1cc(F)cc(CC2CC3(C2)CN(C(=O)N2CC4(CC(n5cnc(C6(O)CC6)n5)C4)C2)C3)c1] | [6-[(3-fluoro-5- methylsulfonyl- phenyl)methyl]-2- azaspiro[3.3]heptan-2- yl]-[6-[3-(1- hydroxycyclopropyl)- 1,2,4-triazol-1-yl]-2- azaspiro[3.3]heptan-2- yl]methanone | A.10 and D.9 | 530.4 [M + H]⁺ |
| 412 | 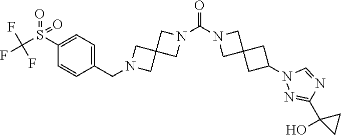 [SMILES: O=C(N1CC2(CC(n3cnc(C4(O)CC4)n3)C2)C1)N1CC2(CN(Cc3ccc(S(=O)(=O)C(F)(F)F)cc3)C2)C1] | [6-[3-(1- hydroxycyclopropyl)- 1,2,4-triazol-1-yl]-2- azaspiro[3.3]heptan-2- yl]-[6-[[4- (trifluoromethylsulfonyl) phenyl]methyl]-2,6- diazaspiro[3.3]heptan- 2-yl]methanone | A.10 and D.54 | 567.4 [M + H]⁺ |
| 413 | 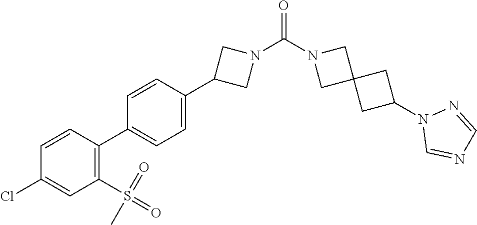 [SMILES: CS(=O)(=O)c1cc(Cl)ccc1-c1ccc(C2CN(C(=O)N3CC4(CC(n5cncn5)C4)C3)C2)cc1] | [3-[4-(4-chloro-2- methylsulfonyl- phenyl)phenyl]azetidin- 1-yl]-[6-(1,2,4-triazol- 1-yl)-2- azaspiro[3.3]heptan-2- yl]methanone | A.17 and D.225 | 512.3 [M + H]⁺ |
| 414 | 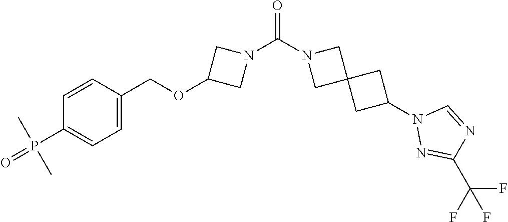 [SMILES: CP(C)(=O)c1ccc(COC2CN(C(=O)N3CC4(CC(n5cnc(C(F)(F)F)n5)C4)C3)C2)cc1] | [3-[(4- dimethyl- phosphorylphenyl) methoxy]azetidin- 1-yl]-[6-[3- (trifluoromethyl)-1,2,4- triazol-1-yl]-2- azaspiro[3.3]heptan-2- yl]methanone | A.13 and E.4 | 498.3 [M + H]⁺ |
| 415 | 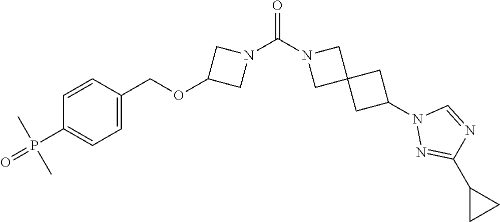 [SMILES: CP(C)(=O)c1ccc(COC2CN(C(=O)N3CC4(CC(n5cnc(C6CC6)n5)C4)C3)C2)cc1] | [6-(3-cyclopropyl- 1,2,4-triazol-1-yl)-2- azaspiro[3.3]heptan-2- yl]-[3-[(4- dimethylphosphorylphe nyl)methoxy]azetidin- 1-yl]methanone | A.1 and E.4 | 470.0 [M + H]⁺ |
| 417 | 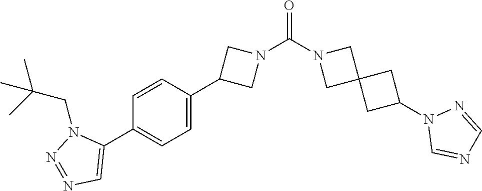 | [3-[4-[3-(2,2- dimethylpropyl)triazol- 4-yl]phenyl]azetidin-1- yl]-[6-(1,2,4-triazol-1- yl)-2- azaspiro[3.3]heptan-2- yl]methanone | A.17 and D.280 | 413.3 [M + H]⁺ |
| 418 | 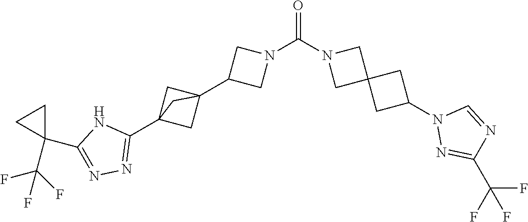 [SMILES: O=C(N1CC(C23CC(c4nnc(C5(C(F)(F)F)CC5)[nH]4)(C2)C3)C1)N1CC2(CC(n3cnc(C(F)(F)F)n3)C2)C1] | [3-[3-[5-[1- (trifluoromethyl) cyclopropyl]- 4H-1,2,4-triazol- 3-yl]-1- bicyclo[1.1.1]pentanyl] azetidin-1-yl]-[6-[3- (trifluoromethyl)-1,2,4- triazol-1-yl]-2- azaspiro[3.3]heptan-2- yl]methanone | A.13 and E.6 | 557.4 [M + H]⁺ |
| 419 | 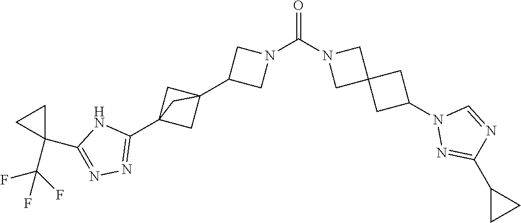 [SMILES: O=C(N1CC(C23CC(c4nnc(C5(C(F)(F)F)CC5)[nH]4)(C2)C3)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[3-[5-[1- (trifluoromethyl) cyclopropyl]-4H- 1,2,4-triazol- 3-yl]-1- bicyclo[1.1.1]pentanyl] azetidin-1- yl]methanone | A.1 and E.6 | 529.5 [M + H]⁺ |
| 420 | 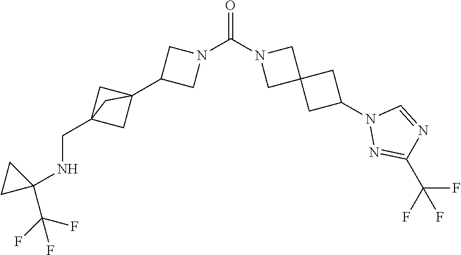 [SMILES: O=C(N1CC(C23CC(CNC4(C(F)(F)F)CC4)(C2)C3)C1)N1CC2(CC(n3cnc(C(F)(F)F)n3)C2)C1] | [3-[3-[[[1- (trifluoromethyl) cyclopropyl]amino] methyl]-1- bicyclo[1.1.1]pentanyl] azetidin-1-yl]-[6-[3- (trifluoromethyl)-1,2,4- triazol-1-yl]-2- azaspiro[3.3]heptan-2- yl]methanone | A.13 and D.255 | 519.5 [M + H]⁺ |
| 421 | 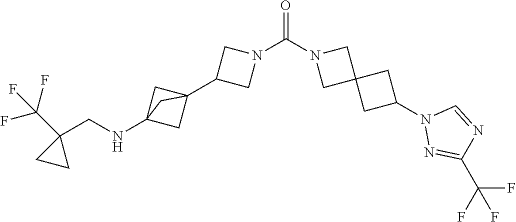 [SMILES: O=C(N1CC(C23CC(NCC4(C(F)(F)F)CC4)(C2)C3)C1)N1CC2(CC(n3cnc(C(F)(F)F)n3)C2)C1] | [3-[3-[[1- (trifluoromethyl) cyclopropyl] methylamino]-1- bicyclo[1.1.1]pentanyl] azetidin-1-yl]-[6-[3- (trifluoromethyl)-1,2,4- triazol-1-yl]-2- azaspiro[3.3]heptan-2- yl]methanone | A.13 and D.256 | 519.5 [M + H]⁺ |
| 422 | 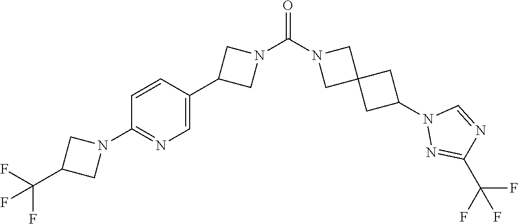 [SMILES: O=C(N1CC(c2ccc(N3CC(C(F)(F)F)C3)nc2)C1)N1CC2(CC(n3cnc(C(F)(F)F)n3)C2)C1] | [3-[6-[3- (trifluoromethyl)azetidin- 1-yl]-3- pyridyl]azetidin-1-yl]- [6-[3-(trifluoromethyl)- 1,2,4-triazol-1-yl]-2- azaspiro[3.3]heptan-2- yl]methanone | A.13 and D.183 | 516.4 [M + H]⁺ |
| 423 | 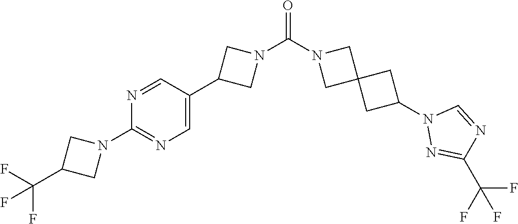 [SMILES: O=C(N1CC(c2cnc(N3CC(C(F)(F)F)C3)nc2)C1)N1CC2(CC(n3cnc(C(F)(F)F)n3)C2)C1] | [3-[2-[3- (trifluoromethyl)azetidin- 1-yl]pyrimidin-5- yl]azetidin-1-yl]-[6-[3- (trifluoromethyl)-1,2,4- triazol-1-yl]-2- azaspiro[3.3]heptan-2- yl]methanone | A.13 and D.185 | 517.4 [M + H]⁺ |
| 424 | 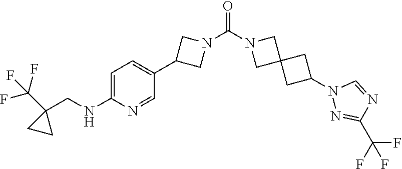 [SMILES: O=C(N1CC(c2ccc(NCC3(C(F)(F)F)CC3)nc2)C1)N1CC2(CC(n3cnc(C(F)(F)F)n3)C2)C1] | [3-[6-[[1- (trifluoromethyl) cyclopropyl] methylamino]-3- pyridyl]azetidin-1-yl]- [6-[3-(trifluoromethyl)- 1,2,4-triazol-1-yl]-2- azaspiro[3.3]heptan-2- yl]methanone | A.13 and D.179 | 530.4 [M + H]⁺ |
| 426 | 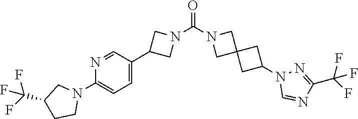 [SMILES: O=C(N1CC(c2ccc(N3CC[C@H](C(F)(F)F)C3)nc2)C1)N1CC2(CC(n3cnc(C(F)(F)F)n3)C2)C1] | [3-[6-[(3S)-3- (trifluoromethyl) pyrrolidin-1-yl]-3- pyridyl]azetidin-1-yl]- [6-[3-(trifluoromethyl)- 1,2,4-triazol-1-yl]-2- azaspiro[3.3]heptan-2- yl]methanone | A.13 and D.187 | 530.4 [M + H]⁺ |
| 427 | 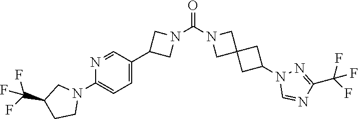 [SMILES: O=C(N1CC(c2ccc(N3CC[C@@H](C(F)(F)F)C3)nc2)C1)N1CC2(CC(n3cnc(C(F)(F)F)n3)C2)C1] | [3-[6-[(3R)-3- (trifluoromethyl) pyrrolidin-1-yl]-3- pyridyl]azetidin-1-yl]- [6-[3-(trifluoromethyl)- 1,2,4-triazol-1-yl]-2- azaspiro[3.3]heptan-2- yl]methanone | A.13 and D.188 | 530.4 [M + H]⁺ |
| 429 | 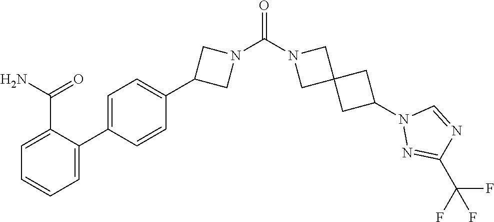 [SMILES: NC(=O)c1ccccc1-c1ccc(C2CN(C(=O)N3CC4(CC(n5cnc(C(F)(F)F)n5)C4)C3)C2)cc1] | 2-[4-[1-[6-[3- (trifluoromethyl)-1,2,4- triazol-1-yl]-2- azaspiro[3.3]heptane-2- carbonyl]azetidin-3- yl]phenyl]benzamide | A.13 and D.254 | 511.4 [M + H]⁺ |
| 430 | 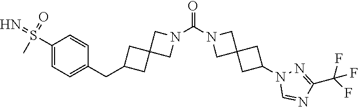 [SMILES: CS(=N)(=O)c1ccc(CC2CC3(C2)CN(C(=O)N2CC4(CC(n5cnc(C(F)(F)F)n5)C4)C2)C3)cc1] | [6-[[4- (methylsulfonimidoyl) phenyl]methyl]-2- azaspiro[3.3]heptan-2- yl]-[6-[3- (trifluoromethyl)-1,2,4- triazol-1-yl]-2- | A.13 and D.78 | 523.4 [M + H]⁺ |
| | | azaspiro[3.3]heptan-2- | | |
| | | yl]methanone | | |
| 431 | 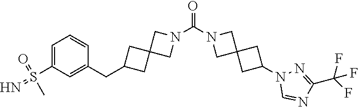 [SMILES: CS(=N)(=O)c1cccc(CC2CC3(C2)CN(C(=O)N2CC4(CC(n5cnc(C(F)(F)F)n5)C4)C2)C3)c1] | [6-[[3- (methylsulfonimidoyl) phenyl]methyl]-2- azaspiro[3.3]heptan-2- yl]-[6-[3- (trifluoromethyl)-1,2,4- triazol-1-yl]-2- | A.13 and D.79 | 523.4 [M + H]⁺ |
| | | azaspiro[3.3]heptan-2- | | |
| | | yl]methanone | | |
| 432 | 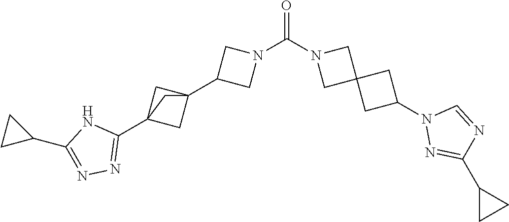 [SMILES: O=C(N1CC(C23CC(c4nnc(C5CC5)[nH]4)(C2)C3)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[3-(5- cyclopropyl-4H-1,2,4- triazol-3-yl)-1- bicyclo[1.1.1]pentanyl] azetidin-1- yl]methanone | A.1 and E.5 | 461.5 [M + H]⁺ |
| 433 | 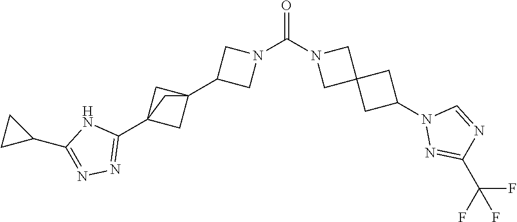 [SMILES: O=C(N1CC(C23CC(c4nnc(C5CC5)[nH]4)(C2)C3)C1)N1CC2(CC(n3cnc(C(F)(F)F)n3)C2)C1] | [3-[3-(5-cyclopropyl- 4H-1,2,4-triazol-3-yl)-1- bicyclo[1.1.1]pentanyl] azetidin-1-yl]-[6-[3- (trifluoromethyl)-1,2,4- triazol-1-yl]-2- azaspiro[3.3]heptan-2- yl]methanone | A.13 and E.5 | 489.4 [M + H]⁺ |
| 435 | 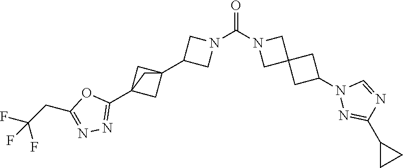 [SMILES: O=C(N1CC(C23CC(c4nnc(CC(F)(F)F)o4)(C2)C3)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[3-[5-(2,2,2- trifluoroethyl)-1,3,4- oxadiazol-2-yl]-1- bicyclo[1.1.1]pentanyl] azetidin-1- yl]methanone | A.1 and D.258 | 504.4 [M + H]⁺ |
| 436 | 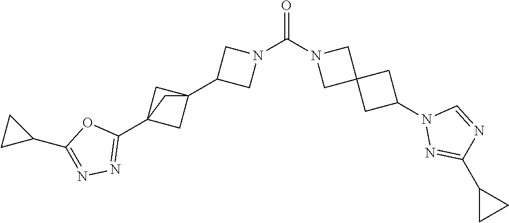 [SMILES: O=C(N1CC(C23CC(c4nnc(C5CC5)o4)(C2)C3)C1)N1CC2(CC(n3cnc(C4CC4)n3)C2)C1] | [3-[3-(5-cyclopropyl- 1,3,4-oxadiazol-2-yl)-1- bicyclo[1.1.1]pentanyl] azetidin-1-yl]-[6-(3- cyclopropyl-1,2,4- triazol-1-yl)-2- azaspiro[3.3]heptan-2- yl]methanone | A.1 and D.259 | 462.4 [M + H]⁺ |
| 437 | 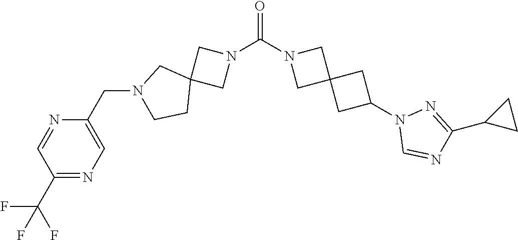 [SMILES: O=C(N1CC2(CC(n3cnc(C4CC4)n3)C2)C1)N1CC2(CCN(Cc3cnc(C(F)(F)F)cn3)C2)C1] | [6-(3-cyclopropyl- 1,2,4-triazol-1-yl)-2- azaspiro[3.3]heptan-2- yl]-[7-[[5- (trifluoromethyl)pyrazin- 2-yl\|methyl]-2,7- diazaspiro[3.4]octan-2- yl]methanone | A.1 and D.124 | 503.4 [M + H]⁺ |
| 438 | 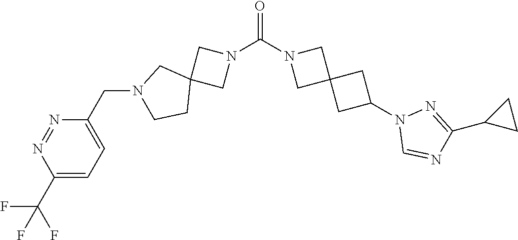 [SMILES: O=C(N1CC2(CC(n3cnc(C4CC4)n3)C2)C1)N1CC2(CCN(Cc3ccc(C(F)(F)F)nn3)C2)C1] | [6-(3-cyclopropyl- 1,2,4-triazol-1-yl)-2- azaspiro[3.3]heptan-2- yl]-[7-[[6- (trifluoromethyl) pyridazin- 3-yl]methyl]-2,7- diazaspiro[3.4]octan-2- yl]methanone | A.1 and D.123 | 503.5 [M + H]⁺ |
| 439 | 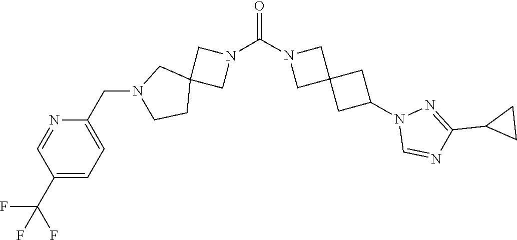 [SMILES: O=C(N1CC2(CC(n3cnc(C4CC4)n3)C2)C1)N1CC2(CCN(Cc3ccc(C(F)(F)F)cn3)C2)C1] | [6-(3-cyclopropyl- 1,2,4-triazol-1-yl)-2- azaspiro[3.3]heptan-2- yl]-[7-[[5- (trifluoromethyl)-2- pyridyl]methyl]-2,7- diazaspiro[3.4]octan-2- yl]methanone | A.1 and D.122 | 502.4 [M + H]⁺ |
| 440 | 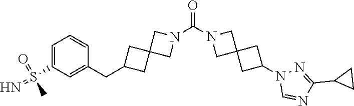 [SMILES: C[S@@](=N)(=O)c1cccc(CC2CC3(C2)CN(C(=O)N2CC4(CC(n5cnc(C6CC6)n5)C4)C2)C3)c1] (enantiomer 1: arbitrary | [6-(3-cyclopropyl- 1,2,4-triazol-1-yl)-2- azaspiro[3.3]heptan-2- yl]-[6-[[3- (methylsulfonimidoyl) phenyl]methyl]-2- azaspiro[3.3]heptan-2- yl]methanone | A.1 and D.79 (chiral separation of racemic Ex. 461) | 495.4 [M + H]⁺ |
| | assignment of stereochemistry) | | | |
| 441 | 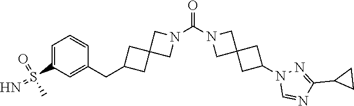 [SMILES: C[S@](=N)(=O)c1cccc(CC2CC3(C2)CN(C(=O)N2CC4(CC(n5cnc(C6CC6)n5)C4)C2)C3)c1] (enantiomer 2: arbitrary | [6-(3-cyclopropyl- 1,2,4-triazol-1-yl)-2- azaspiro[3.3]heptan-2- yl]-[6-[[3- (methylsulfonimidoyl) phenyl]methyl]-2- azaspiro[3.3]heptan-2- yl]methanone | A.1 and D.79 (chiral separation of racemic Ex. 461) | 495.4 [M + H]⁺ |
| | assignment of stereochemistry) | | | |
| 442 | 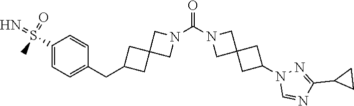 [SMILES: C[S@@](=N)(=O)c1ccc(CC2CC3(C2)CN(C(=O)N2CC4(CC(n5cnc(C6CC6)n5)C4)C2)C3)cc1] (enantiomer 2: arbitrary | [6-(3-cyclopropyl- 1,2,4-triazol-1-yl)-2- azaspiro[3.3]heptan-2- yl]-[6-[[4- (methylsulfonimidoyl)p henyl]methyl]-2- azaspiro[3.3]heptan-2- yl]methanone | A.1 and D.78 (chiral separation of racemic Ex. 462) | 495.4 [M + H]⁺ |
| | assignment of stereochemistry) | | | |
| 443 | 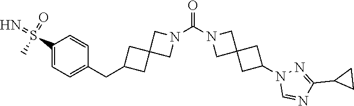 [SMILES: C[S@](=N)(=O)c1ccc(CC2CC3(C2)CN(C(=O)N2CC4(CC(n5cnc(C6CC6)n5)C4)C2)C3)cc1] (enantiomer 1: arbitrary | [6-(3-cyclopropyl- 1,2,4-triazol-1-yl)-2- azaspiro[3.3]heptan-2- yl]-[6-[[4- (methylsulfonimidoyl) phenyl]methyl]-2- azaspiro[3.3]heptan-2- yl]methanone | A.1 and D.78 (chiral separation of racemic Ex. 462) | 495.4 [M + H]⁺ |
| | assignment of stereochemistry) | | | |
| 444 | 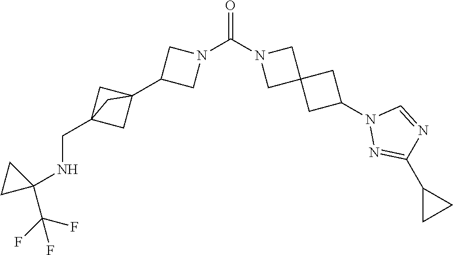 [SMILES: O=C(N1CC(C23CC(CNC4(C(F)(F)F)CC4)(C2)C3)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[3-[[[1- (trifluoromethyl) cyclopropyl] amino]methyl]-1- bicyclo[1.1.1]pentanyl] azetidin-1- yl]methanone | A.1 and D.255 | 491.4 [M + H]⁺ |
| 445 | 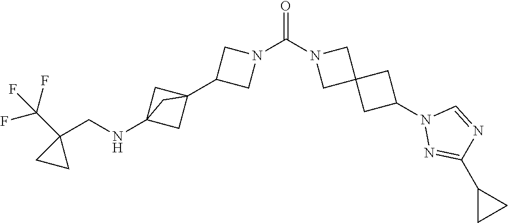 [SMILES: O=C(N1CC(C23CC(NCC4(C(F)(F)F)CC4)(C2)C3)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[3-[[1- (trifluoromethyl) cyclopropyl] methylamino]-1- bicyclo[1.1.1]pentanyl] azetidin-1- yl]methanone | A.1 and D.256 | 491.5 [M + H]⁺ |
| 449 | 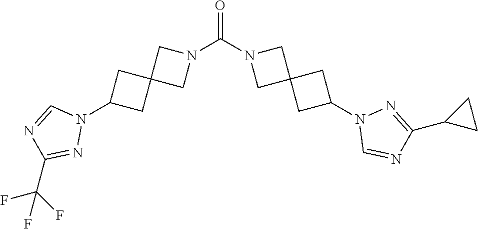 [SMILES: O=C(N1CC2(CC(n3cnc(C4CC4)n3)C2)C1)N1CC2(CC(n3cnc(C(F)(F)F)n3)C2)C1] | [6-(3-cyclopropyl- 1,2,4-triazol-1-yl)-2- azaspiro[3.3]heptan-2- yl]-[6-[3- (trifluoromethyl)-1,2,4- triazol-1-yl]-2- azaspiro[3.3]heptan-2- yl]methanone | A.1 and A.13 | 463.4 [M + H]⁺ |
| 451 | 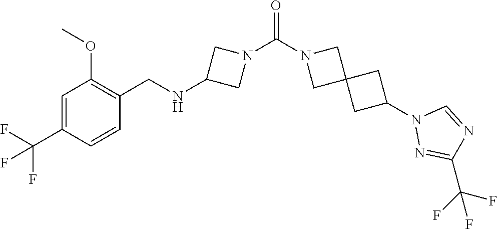 [SMILES: COc1cc(C(F)(F)F)ccc1CNC1CN(C(=O)N2CC3(CC(n4cnc(C(F)(F)F)n4)C3)C2)C1] | [3-[[2-methoxy-4- (trifluoromethyl)phenyl] methylamino]azetidin- 1-yl]-[6-[3- (trifluoromethyl)-1,2,4- triazol-1-yl]-2- azaspiro[3.3]heptan-2- yl]methanone | A.13 and D.137 | 519.4 [M + H]⁺ |
| 452 | 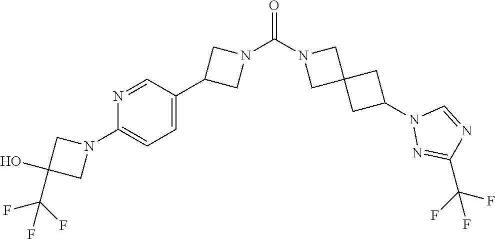 [SMILES: O=C(N1CC(c2ccc(N3CC(O)(C(F)(F)F)C3)nc2)C1)N1CC2(CC(n3cnc(C(F)(F)F)n3)C2)C1] | [3-[6-[3-hydroxy-3- (trifluoromethyl)azetidin- 1-yl]-3- pyridyl]azetidin-1-yl]- [6-[3-(trifluoromethyl)- 1,2,4-triazol-1-yl]-2- azaspiro[3.3]heptan-2- yl]methanone | A.13 and D.196 | 532.3 [M + H]⁺ |
| 454 | 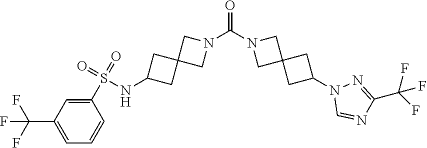 [SMILES: O=C(N1CC2(CC(NS(=O)(=O)c3cccc(C(F)(F)F)c3)C2)C1)N1CC2(CC(n3cnc(C(F)(F)F)n3)C2)C1] | 3-(trifluoromethyl)-N- [2-[6-[3- (trifluoromethyl)-1,2,4- triazol-1-yl]-2- azaspiro[3.3]heptane-2- carbonyl]-2- azaspiro[3.3]heptan-6- yl]benzenesulfonamide | A.13 and D.30 | 551.3 [M + H]⁺ |
| 455 | 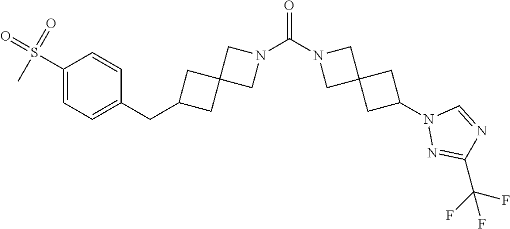 [SMILES: CS(=O)(=O)c1ccc(CC2CC3(C2)CN(C(=O)N2CC4(CC(n5cnc(C(F)(F)F)n5)C4)C2)C3)cc1] | [6-[(4- methylsulfonylphenyl) methyl]-2- azaspiro[3.3]heptan-2- yl]-[6-[3- (trifluoromethyl)-1,2,4- triazol-1-yl]-2- azaspiro[3.3]heptan-2- yl]methanone | A.13 and D.7 | 524.4 [M + H]⁺ |
| 456 | 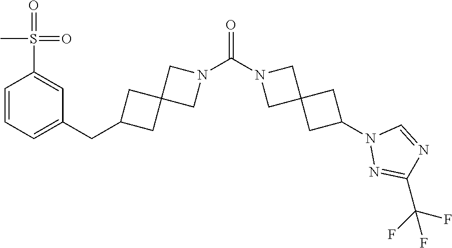 [SMILES: CS(=O)(=O)c1cccc(CC2CC3(C2)CN(C(=O)N2CC4(CC(n5cnc(C(F)(F)F)n5)C4)C2)C3)c1] | [6-[(3- methylsulfonylphenyl) methyl]-2- azaspiro[3.3]heptan-2- yl]-[6-[3- (trifluoromethyl)-1,2,4- triazol-1-yl]-2- azaspiro[3.3]heptan-2- yl]methanone | A.13 and D.8 | 524.4 [M + H]⁺ |
| 457 | 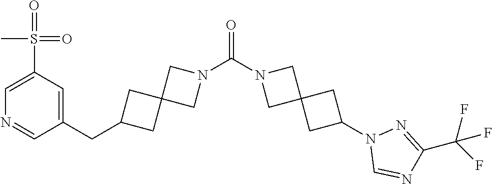 [SMILES: CS(=O)(=O)c1cncc(CC2CC3(C2)CN(C(=O)N2CC4(CC(n5cnc(C(F)(F)F)n5)C4)C2)C3)c1] | [6-[(5-methylsulfonyl- 3-pyridyl)methyl]-2- azaspiro[3.3]heptan-2- yl]-[6-[3- (trifluoromethyl)-1,2,4- triazol-1-yl]-2- azaspiro[3.3]heptan-2- yl]methanone | A.13 and D.76 | 525.4 [M + H]⁺ |
| 458 | 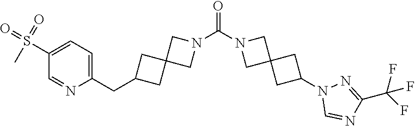 [SMILES: CS(=O)(=O)c1ccc(CC2CC3(C2)CN(C(=O)N2CC4(CC(n5cnc(C(F)(F)F)n5)C4)C2)C3)nc1] | [6-[(5-methylsulfonyl- 2-pyridyl)methyl]-2- azaspiro[3.3]heptan-2- yl]-[6-[3- (trifluoromethyl)-1,2,4- triazol-1-yl]-2- azaspiro[3.3]heptan-2- yl]methanone | A.13 and D.75 | 525.3 [M + H]⁺ |
| 459 | 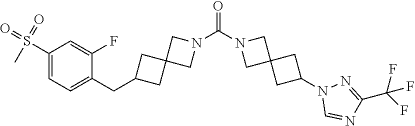 [SMILES: CS(=O)(=O)c1ccc(CC2CC3(C2)CN(C(=O)N2CC4(CC(n5cnc(C(F)(F)F)n5)C4)C2)C3)c(F)c1] | [6-[(2-fluoro-4- methylsulfonyl- phenyl)methyl]-2- azaspiro[3.3]heptan-2- yl]-[6-[3- (trifluoromethyl)-1,2,4- triazol-1-yl]-2- azaspiro[3.3]heptan-2- | A.13 and D.82 | 542.3 [M + H]⁺ |
| | | yl]methanone | | |
| 460 | 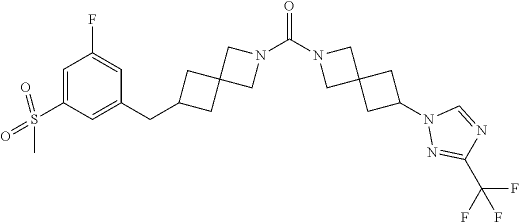 [SMILES: CS(=O)(=O)c1cc(F)cc(CC2CC3(C2)CN(C(=O)N2CC4(CC(n5cnc(C(F)(F)F)n5)C4)C2)C3)c1] | [6-[(3-fluoro-5- methylsulfonyl- phenyl)methyl]-2- azaspiro[3.3]heptan-2- yl]-[6-[3- (trifluoromethyl)-1,2,4- triazol-1-yl]-2- azaspiro[3.3]heptan-2- yl]methanone | A.13 and D.9 | 542.3 [M + H]⁺ |
| 461 | 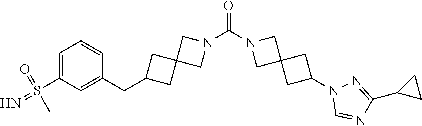 [SMILES: CS(=N)(=O)c1cccc(CC2CC3(C2)CN(C(=O)N2CC4(CC(n5cnc(C6CC6)n5)C4)C2)C3)c1] | [6-(3-cyclopropyl- 1,2,4-triazol-1-yl)-2- azaspiro[3.3]heptan-2- yl]-[6-[[3- (methylsulfonimidoyl) phenyl]methyl]-2- azaspiro[3.3]heptan-2- yl]methanone | A.1 and D.79 | 495.3 [M + H]⁺ |
| 462 | 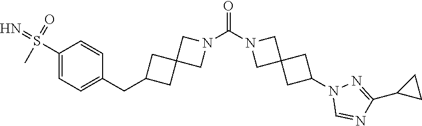 [SMILES: CS(=N)(=O)c1ccc(CC2CC3(C2)CN(C(=O)N2CC4(CC(n5cnc(C6CC6)n5)C4)C2)C3)cc1] | [6-(3-cyclopropyl- 1,2,4-triazol-1-yl)-2- azaspiro[3.3]heptan-2- yl]-[6-[[4- (methylsulfonimidoyl) phenyl]methyl]-2- azaspiro[3.3]heptan-2- yl]methanone | A.1 and D.78 | 495.3 [M + H]⁺ |
| 463 | 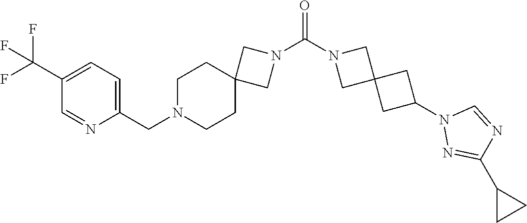 [SMILES: O=C(N1CC2(CCN(Cc3ccc(C(F)(F)F)cn3)CC2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[7-[[5- (trifluoromethyl)-2- pyridyl]methyl]-2,7- diazaspiro[3.5]nonan-2- yl]methanone | A.1 and D.118 | 516.3 [M + H]⁺ |
| 464 | 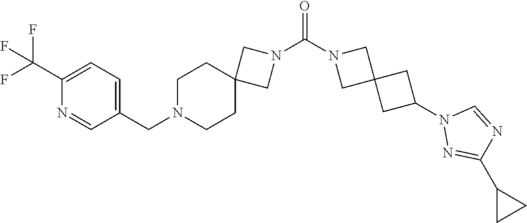 [SMILES: O=C(N1CC2(CCN(Cc3ccc(C(F)(F)F)nc3)CC2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[7-[[6- (trifluoromethyl)-3- pyridyl]methyl]-2,7- diazaspiro[3.5]nonan-2- yl]methanone | A.1 and D.116 | 516.3 [M + H]⁺ |
| 465 | 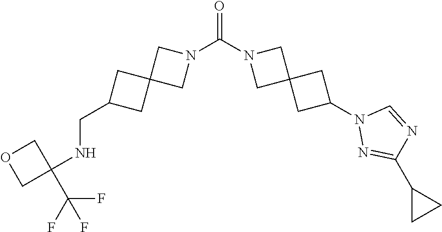 [SMILES: O=C(N1CC2(CC(CNC3(C(F)(F)F)COC3)C2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[6-[[[3- (trifluoromethyl)oxetan- 3-yl]amino]methyl]-2- azaspiro[3.3]heptan-2- yl]methanone | A.1 and D.268 | 481.4 [M + H]⁺ |
| 466 | 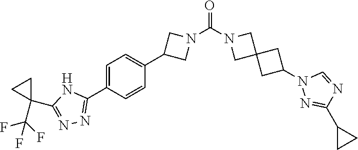 [SMILES: O=C(N1CC(c2ccc(-c3nnc(C4(C(F)(F)F)CC4)[nH]3)cc2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[4-[5-[1- (trifluoromethyl) cyclopropyl]-4H- 1,2,4-triazol- 3-yl]phenyl]azetidin-1- yl]methanone | A.1 and D.287 | 539.4 [M + H]⁺ |
| 468 | 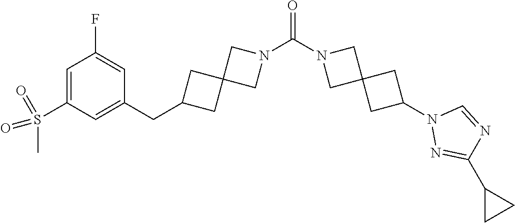 [SMILES: CS(=O)(=O)c1cc(F)cc(CC2CC3(C2)CN(C(=O)N2CC4(CC(n5cnc(C6CC6)n5)C4)C2)C3)c1] | [6-(3-cyclopropyl- 1,2,4-triazol-1-yl)-2- azaspiro[3.3]heptan-2- yl]-[6-[(3-fluoro-5- methylsulfonyl- phenyl)methyl]-2- azaspiro[3.3]heptan-2- yl]methanone | A.1 and D.9 | 514.4 [M + H]⁺ |
| 469 | 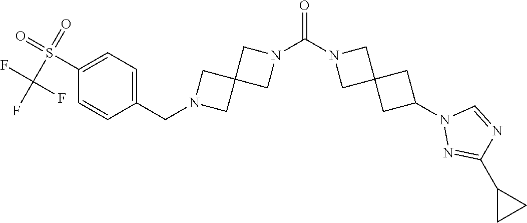 [SMILES: O=C(N1CC2(CC(n3cnc(C4CC4)n3)C2)C1)N1CC2(CN(Cc3ccc(S(=O)(=O)C(F)(F)F)cc3)C2)C1] | [6-(3-cyclopropyl- 1,2,4-triazol-1-yl)-2- azaspiro[3.3]heptan-2- yl]-[6-[[4- (trifluoromethylsulfonyl) phenyl]methyl]-2,6- diazaspiro[3.3]heptan- 2-yl]methanone | A.1 and D.54 | 551.3 [M + H]⁺ |
| 470 | 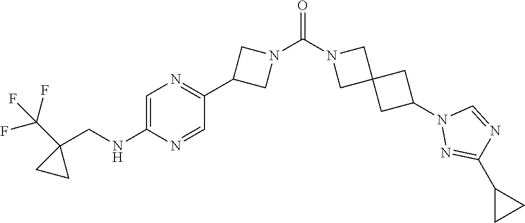 [SMILES: O=C(N1CC(c2cnc(NCC3(C(F)(F)F)CC3)cn2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[5-[[1- (trifluoromethyl) cyclopropyl] methylamino] pyrazin-2-yl]azetidin-1- yl]methanone | A.1 and D.180 | 503.3 [M + H]⁺ |
| 472 | 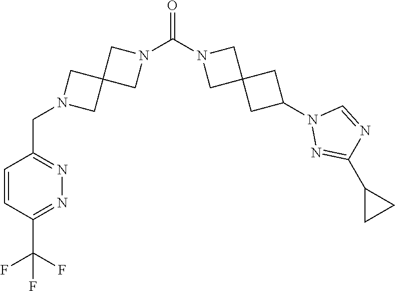 [SMILES: O=C(N1CC2(CC(n3cnc(C4CC4)n3)C2)C1)N1CC2(CN(Cc3ccc(C(F)(F)F)nn3)C2)C1] | [6-(3-cyclopropyl- 1,2,4-triazol-1-yl)-2- azaspiro[3.3]heptan-2- yl]-[6-[[6- (trifluoromethyl) pyridazin- 3-yl]methyl]-2,6- diazaspiro[3.3]heptan- 2-yl]methanone | A.1 and D.101 | 489.3 [M + H]⁺ |
| 473 | 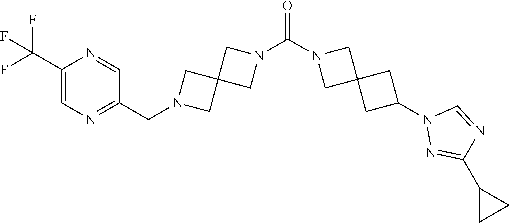 [SMILES: O=C(N1CC2(CC(n3cnc(C4CC4)n3)C2)C1)N1CC2(CN(Cc3cnc(C(F)(F)F)cn3)C2)C1] | [6-(3-cyclopropyl- 1,2,4-triazol-1-yl)-2- azaspiro[3.3]heptan-2- yl]-[6-[[5- (trifluoromethyl) pyrazin- 2-yl]methyl]-2,6- diazaspiro[3.3]heptan- 2-yl]methanone | A.1 and D.105 | 489.3 [M + H]⁺ |
| 474 | 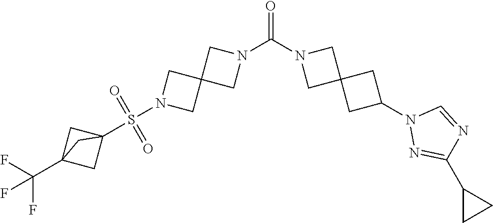 [SMILES: O=C(N1CC2(CC(n3cnc(C4CC4)n3)C2)C1)N1CC2(C1)CN(S(=O)(=O)C13CC(C(F)(F)F)(C1)C3)C2] | [6-(3-cyclopropyl- 1,2,4-triazol-1-yl)-2- azaspiro[3.3]heptan-2- yl]-[2-[[3- (trifluoromethyl)-1- bicyclo[1.1.1]pentanyl] sulfonyl]-2,6- diazaspiro[3.3]heptan- 6-yl]methanone | A.1 and D.33 | 527.2 [M + H]⁺ |
| 475 | 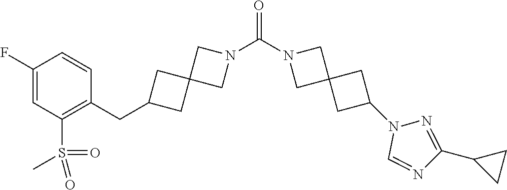 [SMILES: CS(=O)(=O)c1cc(F)ccc1CC1CC2(C1)CN(C(=O)N1CC3(CC(n4cnc(C5CC5)n4)C3)C1)C2] | [6-(3-cyclopropyl- 1,2,4-triazol-1-yl)-2- azaspiro[3.3]heptan-2- yl]-[6-[(4-fluoro-2- methylsulfonyl- phenyl)methyl]-2- azaspiro[3.3]heptan-2- yl]methanone | A.1 and D.12 | 514.3 [M + H]⁺ |
| 476 | 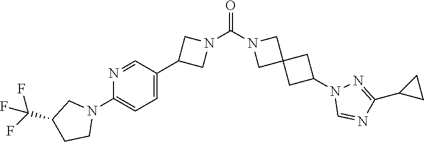 [SMILES: O=C(N1CC(c2ccc(N3CC[C@H](C(F)(F)F)C3)nc2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[6-[(3S)-3- (trifluoromethyl) pyrrolidin-1-yl]-3- pyridyl]azetidin-1- yl]methanone | A.1 and D.187 | 502.3 [M + H]⁺ |
| 477 | 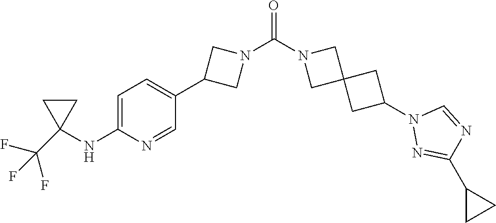 [SMILES: O=C(N1CC(c2ccc(NC3(C(F)(F)F)CC3)nc2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[6-[[1- (trifluoromethyl) cyclopropyl]amino]-3- pyridyl]azetidin-1- yl]methanone | A.1 and D.190 | 488.3 [M + H]⁺ |
| 478 | 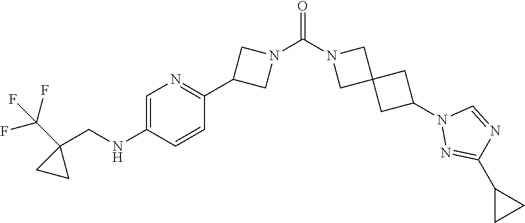 [SMILES: O=C(N1CC(c2ccc(NCC3(C(F)(F)F)CC3)cn2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[5-[[1- (trifluoromethyl) cyclopropyl] methylamino]-2- pyridyl]azetidin-1- yl]methanone | A.1 and D.181 | 502.3 [M + H]⁺ |
| 479 | 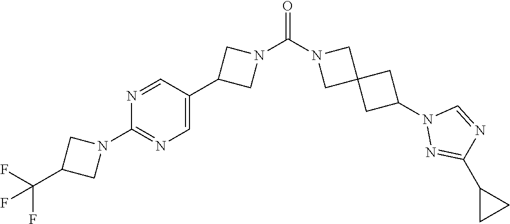 [SMILES: O=C(N1CC(c2cnc(N3CC(C(F)(F)F)C3)nc2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[2-[3- (trifluoromethyl) azetidin- 1-yl]pyrimidin-5- yl]azetidin-1- yl]methanone | A.1 and D.185 | 489.3 [M + H]⁺ |
| 480 | 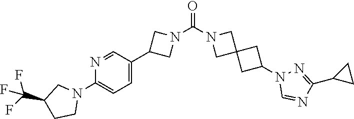 [SMILES: O=C(N1CC(c2ccc(N3CC[C@@H](C(F)(F)F)C3)nc2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[6-[(3S)-3- (trifluoromethyl) pyrrolidin-1-yl]-3- pyridyl]azetidin-1- yl]methanone | A.1 and D.188 | 502.3 [M + H]⁺ |
| 481 | 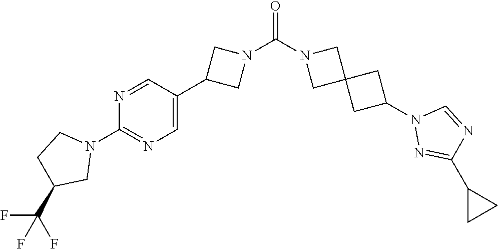 [SMILES: O=C(N1CC(c2cnc(N3CC[C@H](C(F)(F)F)C3)nc2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[2-[(3S)-3- (trifluoromethyl) pyrrolidin- 1-yl]pyrimidin-5- yl]azetidin-1- yl]methanone | A.1 and D.187 | 503.3 [M + H]⁺ |
| 482 | 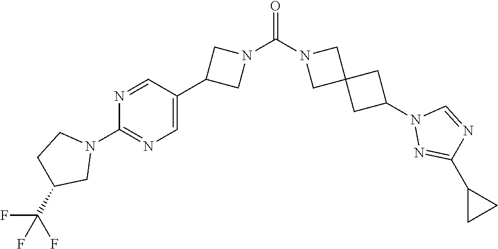 [SMILES: O=C(N1CC(c2cnc(N3CC[C@@H](C(F)(F)F)C3)nc2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[2-[(3R)-3- (trifluoromethyl) pyrrolidin- 1-yl]pyrimidin-5- yl]azetidin-1- yl]methanone | A.1 and D.188 | 503.3 [M + H]⁺ |
| 483 | 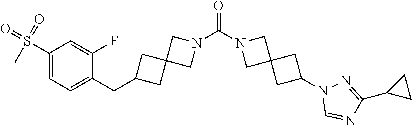 [SMILES: CS(=O)(=O)c1ccc(CC2CC3(C2)CN(C(=O)N2CC4(CC(n5cnc(C6CC6)n5)C4)C2)C3)c(F)c1] | [6-(3-cyclopropyl- 1,2,4-triazol-1-yl)-2- azaspiro[3.3]heptan-2- yl]-[6-[(2-fluoro-4- methylsulfonyl- phenyl)methyl]-2- azaspiro[3.3]heptan-2- yl]methanone | A.1 and D.82 | 514.3 [M + H]⁺ |
| 484 | 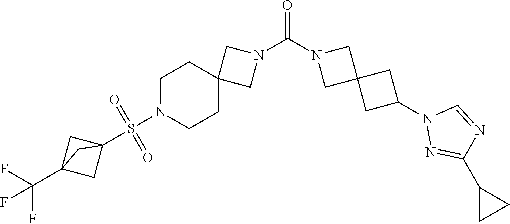 [SMILES: O=C(N1CC2(CCN(S(=O)(=O)C34CC(C(F)(F)F)(C3)C4)CC2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[7-[[3- (trifluoromethyl)-1- bicyclo[1.1.1]pentanyl] sulfonyl]-2,7- diazaspiro[3.5]nonan-2- yl]methanone | A.1 and D.167 | 555.3 [M + H]⁺ |
| 485 | 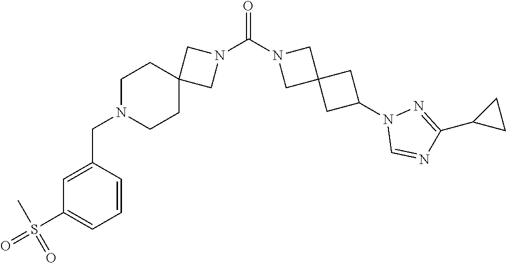 [SMILES: CS(=O)(=O)c1cccc(CN2CCC3(CC2)CN(C(=O)N2CC4(CC(n5cnc(C6CC6)n5)C4)C2)C3)c1] | [6-(3-cyclopropyl- 1,2,4-triazol-1-yl)-2- azaspiro[3.3]heptan-2- yl]-[7-[(3- methylsulfonylphenyl) methyl]-2,7- diazaspiro[3.5]nonan-2- yl]methanone | A.1 and D.121 | 525.3 [M + H]⁺ |
| 486 | 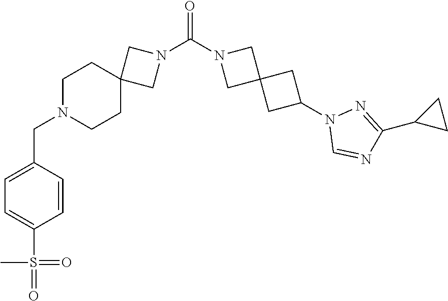 [SMILES: CS(=O)(=O)c1ccc(CN2CCC3(CC2)CN(C(=O)N2CC4(CC(n5cnc(C6CC6)n5)C4)C2)C3)cc1] | [6-(3-cyclopropyl- 1,2,4-triazol-1-yl)-2- azaspiro[3.3]heptan-2- yl]-[7-[(4- methylsulfonylphenyl) methyl]-2,7- diazaspiro[3.5]nonan-2- yl]methanone | A.1 and D.119 | 525.3 [M + H]⁺ |
| 487 | 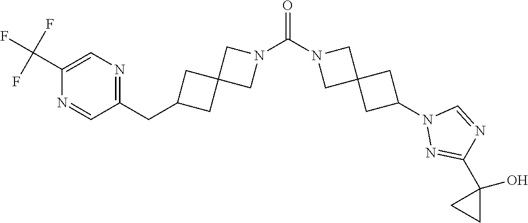 [SMILES: O=C(N1CC2(CC(Cc3cnc(C(F)(F)F)cn3)C2)C1)N1CC2(CC(n3cnc(C4(O)CC4)n3)C2)C1] | [6-[3-(1- hydroxycyclopropyl)- 1,2,4-triazol-1-yl]-2- azaspiro[3.3]heptan-2- yl]-[6-[[5- (trifluoromethyl)pyrazin- 2-yl]methyl]-2- azaspiro[3.3]heptan-2- yl]methanone | A.10 and D.4 | 504.3 [M + H]⁺ |
| 488 | 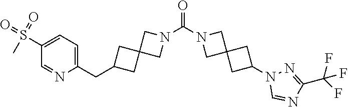 | [6-(3-cyclopropyl- 1,2,4-triazol-1-yl)-2- azaspiro[3.3]heptan-2- yl]-[6-[(5- methylsulfonyl-2- pyridyl)methyl]-2- azaspiro[3.3]heptan-2- | A.1 and D.75 | 497.3 [M + H]⁺ |
| | | yl]methanone | | |
| 489 | 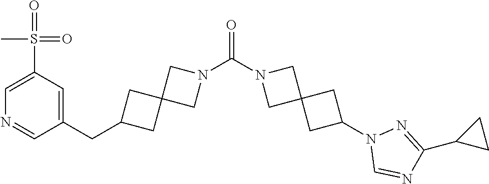 [SMILES: CS(=O)(=O)c1cncc(CC2CC3(C2)CN(C(=O)N2CC4(CC(n5cnc(C6CC6)n5)C4)C2)C3)c1] | [6-(3-cyclopropyl- 1,2,4-triazol-1-yl)-2- azaspiro[3.3]heptan-2- yl]-[6-[(5- methylsulfonyl-3- pyridyl)methyl]-2- azaspiro[3.3]heptan-2- yl]methanone | A.1 and D.76 | 497.3 [M + H]⁺ |
| 490 | 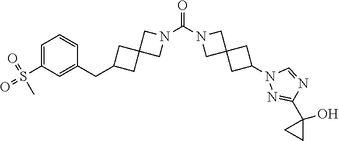 [SMILES: CS(=O)(=O)c1cccc(CC2CC3(C2)CN(C(=O)N2CC4(CC(n5cnc(C6(O)CC6)n5)C4)C2)C3)c1] | [6-[3-(1- hydroxycyclopropyl)- 1,2,4-triazol-1-yl]-2- azaspiro[3.3]heptan-2- yl]-[6-[(3- methylsulfonylphenyl) methyl]-2- azaspiro[3.3]heptan-2- yl]methanone | A.10 and D.8 | 512.3 [M + H]⁺ |
| 491 | 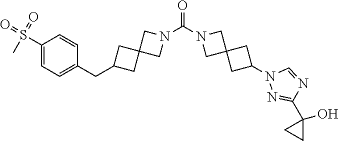 [SMILES: CS(=O)(=O)c1ccc(CC2CC3(C2)CN(C(=O)N2CC4(CC(n5cnc(C6(O)CC6)n5)C4)C2)C3)cc1] | [6-[3-(1- hydroxycyclopropyl)- 1,2,4-triazol-1-yl]-2- azaspiro[3.3]heptan-2- yl]-[6-[(4- methylsulfonylphenyl) methyl]-2- azaspiro[3.3]heptan-2- yl]methanone | A.10 and D.7 | 512.3 [M + H]⁺ |
| 492 | 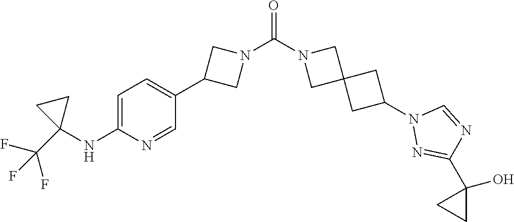 [SMILES: O=C(N1CC(c2ccc(NC3(C(F)(F)F)CC3)nc2)C1)N1CC2(CC(n3cnc(C4(O)CC4)n3)C2)C1] | [6-[3-(1- hydroxycyclopropyl)- 1,2,4-triazol-1-yl]-2- azaspir[3.3]heptan-2- yl]-[3-[6-[[1- (trifluoromethyl)cyclop ropyl]amino]-3- pyridyl]azetidin-1- yl]methanone | A.10 and D.190 | 504.3 [M + H]⁺ |
| 495 | 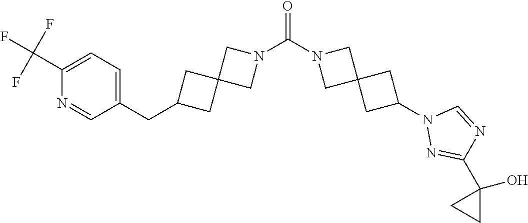 [SMILES: O=C(N1CC2(CC(Cc3ccc(C(F)(F)F)nc3)C2)C1)N1CC2(CC(n3cnc(C4(O)CC4)n3)C2)C1] | [6-[3-(1- hydroxycyclopropyl)- 1,2,4-triazol-1-yl]-2- azaspiro[3.3]heptan-2- yl]-[6-[[6- (trifluoromethyl)-3- pyridyl]methyl]-2- azaspiro[3.3]heptan-2- yl]methanone | A.10 and D.3 | 503.3 [M + H]⁺ |
| 496 | 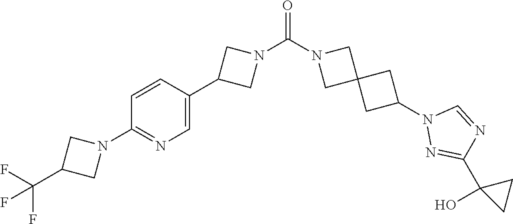 [SMILES: O=C(N1CC(c2ccc(N3CC(C(F)(F)F)C3)nc2)C1)N1CC2(CC(n3cnc(C4(O)CC4)n3)C2)C1] | [6-[3-(1- hydroxycyclopropyl)- 1,2,4-triazol-1-yl]-2- azaspiro[3.3]heptan-2- yl]-[3-[6-[3- (trifluoromethyl) azetidin- 1-yl]-3- pyridyl]azetidin-1- yl]methanone | A.10 and D.183 | 504.3 [M + H]⁺ |
| 497 | 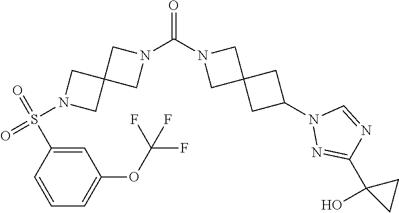 [SMILES: O=C(N1CC2(CC(n3cnc(C4(O)CC4)n3)C2)C1)N1CC2(C1)CN(S(=O)(=O)c1cccc(OC(F)(F)F)c1)C2] | [6-[3-(1- hydroxycyclopropyl)- 1,2,4-triazol-1-yl]-2- azaspiro[3.3]heptan-2- yl]-[2-[3- (trifluoromethoxy)phen yl]sulfonyl-2,6- diazaspiro[3.3]heptan- 6-yl]methanone | A.10 and D.55 | 569.2 [M + H]⁺ |
| 498 | 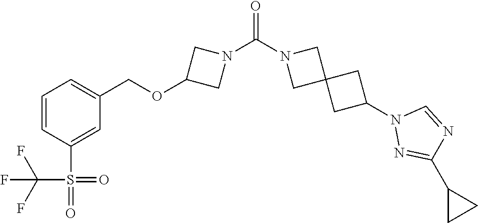 [SMILES: O=C(N1CC(OCc2cccc(S(=O)(=O)C(F)(F)F)c2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[[3- (trifluoromethylsulfonyl) phenyl]methoxy]azetidin- 1-yl]methanone | A.1 and D.237 | 526.3 [M + H]⁺ |
| 499 | 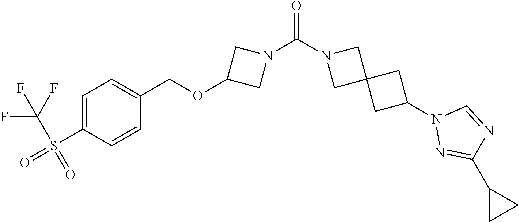 [SMILES: O=C(N1CC(OCc2ccc(S(=O)(=O)C(F)(F)F)cc2)C1)N1CC2(CC(n3cnc(C4CC4)n3)C2)C1] | [6-(3-cyclopropyl- 1,2,4-triazol-1-yl)-2- azaspiro[3.3]heptan-2- yl]-[3-[[4- (trifluoromethylsulfonyl) phenyl]methoxy]azetidin- 1-yl]methanone | A.1 and D.232 | 526.3 [M + H]⁺ |
| 500 | 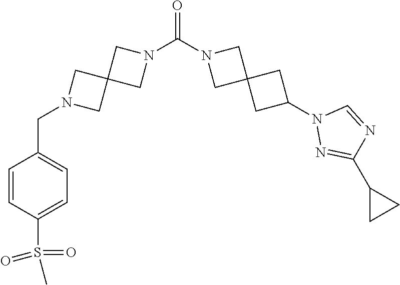 [SMILES: CS(=O)(=O)c1ccc(CN2CC3(C2)CN(C(=O)N2CC4(CC(n5cnc(C6CC6)n5)C4)C2)C3)cc1] | [6-(3-cyclopropyl- 1,2,4-triazol-1-yl)-2- azaspiro[3.3]heptan-2- yl]-[6-[(4- methylsulfonylphenyl) methyl]-2,6- diazaspiro[3.3]heptan- 2-yl]methanone | A.1 and D.107 | 497.3 [M + H]⁺ |
| 501 | 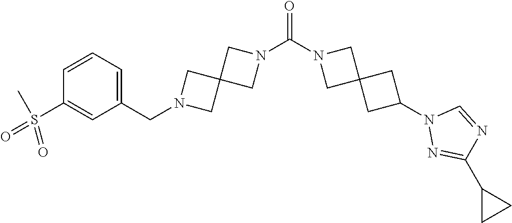 [SMILES: CS(=O)(=O)c1cccc(CN2CC3(C2)CN(C(=O)N2CC4(CC(n5cnc(C6CC6)n5)C4)C2)C3)c1] | [6-(3-cyclopropyl- 1,2,4-triazol-1-yl)-2- azaspiro[3.3]heptan-2- yl]-[6-[(3- methylsulfonylphenyl) methyl]-2,6- diazaspiro[3.3]heptan- 2-yl]methanone | A.1 and D.108 | 497.3 [M + H]⁺ |
| 502 | 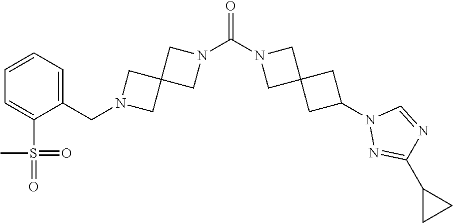 [SMILES: CS(=O)(=O)c1ccccc1CN1CC2(C1)CN(C(=O)N1CC3(CC(n4cnc(C5CC5)n4)C3)C1)C2] | [6-(3-cyclopropyl- 1,2,4-triazol-1-yl)-2- azaspiro[3.3]heptan-2- yl]-[6-[(2- methylsulfonylphenyl) methyl]-2,6- diazaspiro[3.3]heptan- 2-yl]methanone | A.1 and D.109 | 497.3 [M + H]⁺ |
| 503 | 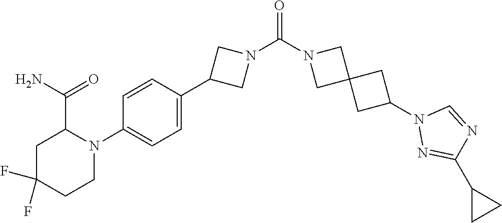 [SMILES: NC(=O)C1CC(F)(F)CCN1c1ccc(C2CN(C(=O)N3CC4(CC(n5cnc(C6CC6)n5)C4)C3)C2)cc1] | 1-[4-[1-[6-(3- cyclopropyl-1,2,4- triazol-1-yl)-2- azaspiro[3.3]heptane-2- carbonyl]azetidin-3- yl]phenyl]-4,4-difluoro- piperidine-2-carboxamide | A.1 and D.294 | 526.3 [M + H]⁺ |
| 510 | 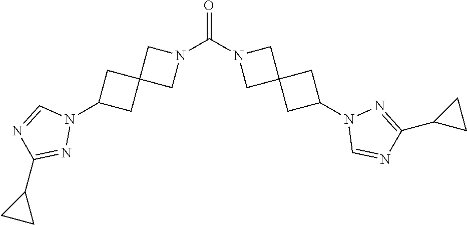 [SMILES: O=C(N1CC2(CC(n3cnc(C4CC4)n3)C2)C1)N1CC2(CC(n3cnc(C4CC4)n3)C2)C1] | bis[6-(3-cyclopropyl- 1,2,4-triazol-1-yl)-2- azaspiro[3.3]heptan-2- yl]methanone | A.1 and A.1 | 435.4 [M + H]⁺ |
| 512 | 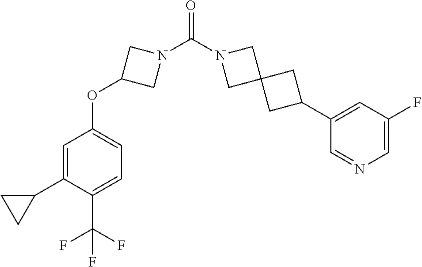 [SMILES: O=C(N1CC(Oc2ccc(C(F)(F)F)c(C3CC3)c2)C1)N1CC2(CC(c3cncc(F)c3)C2)C1] | [3-[3-cyclopropyl-4- (trifluoromethyl) phenoxylazetidin- 1-yl]-[6-(5- fluoro-3-pyridyl)-2- azaspiro[3.3]heptan-2- yl]methanone | A.12 and D.235 | 476.4 [M + H]⁺ |
| 513 | 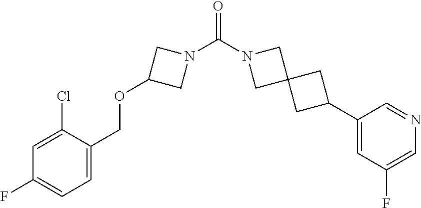 [SMILES: O=C(N1CC(OCc2ccc(F)cc2Cl)C1)N1CC2(CC(c3cncc(F)c3)C2)C1] | [3-[(2-chloro-4-fluoro- phenyl)methoxy]azetidin- 1-yl]-[6-(5-fluoro-3- pyridyl)-2- azaspiro[3.3]heptan-2- yl]methanone | A.12 and B.25 | 434.3 [M + H]⁺ |
| 514 | 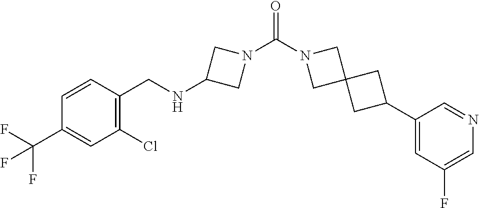 [SMILES: O=C(N1CC(NCc2ccc(C(F)(F)F)cc2Cl)C1)N1CC2(CC(c3cncc(F)c3)C2)C1] | [3-[[2-chloro-4- (trifluoromethyl)phenyl] methylamino]azetidin- 1-yl]-[6-(5-fluoro-3- pyridyl)-2- azaspiro[3.3]heptan-2- yl]methanone | A.12 and D.130 | 483.3 [M + H]⁺ |
| 515 | 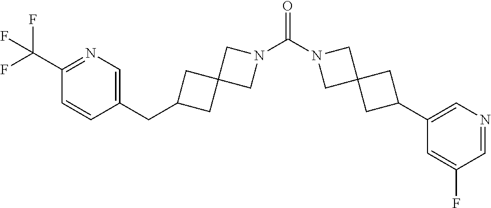 [SMILES: O=C(N1CC2(CC(Cc3ccc(C(F)(F)F)nc3)C2)C1)N1CC2(CC(c3cncc(F)c3)C2)C1] | [6-(5-fluoro-3-pyridyl)- 2-azaspiro[3.3]heptan- 2-yl]-[6-[[6- (trifluoromethyl)-3- pyridyl]methyl]-2- azaspiro[3.3]heptan-2- yl]methanone | A.12 and D.3 | 475.3 [M + H]⁺ |
| 516 | 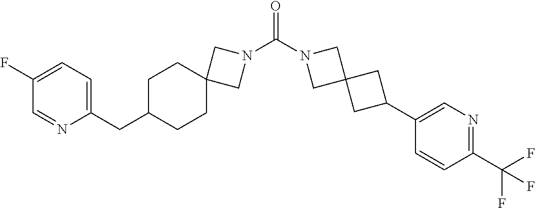 [SMILES: O=C(N1CC2(CCC(Cc3ccc(F)cn3)CC2)C1)N1CC2(CC(c3ccc(C(F)(F)F)nc3)C2)C1] | [7-[(5-fluoro-2- pyridyl)methyl]-2- azaspiro[3.5]nonan-2- yl]-[6-[6- (trifluoromethyl)-3- pyridyl]-2- azaspiro[3.3]heptan-2- yl]methanone | A.11 and D.90 | 503.3 [M + H]⁺ |
| 517 | 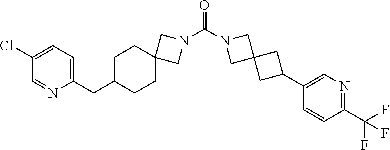 [SMILES: O=C(N1CC2(CCC(Cc3ccc(Cl)cn3)CC2)C1)N1CC2(CC(c3ccc(C(F)(F)F)nc3)C2)C1] | [7-[(5-chloro-2- pyridyl)methyl]-2- azaspiro[3.5]nonan-2- yl]-[6-[6- (trifluoromethyl)-3- pyridyl]-2- azaspiro[3.3]heptan-2- yl]methanone | A.11 and D.87 | 519.3 [M + H]⁺ |
| 518 | 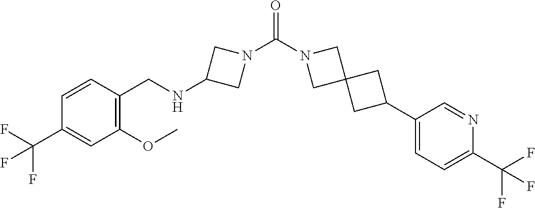 [SMILES: COc1cc(C(F)(F)F)ccc1CNC1CN(C(=O)N2CC3(CC(c4ccc(C(F)(F)F)nc4)C3)C2)C1] | [3-[[2-methoxy-4- (trifluoromethyl)phenyl] methylamino]azetidin- 1-yl]-[6-[6- (trifluoromethyl)-3- pyridyl]-2- azaspiro[3.3]heptan-2- yl]methanone | A.11 and D.518 | 529.3 [M + H]⁺ |
| 519 | 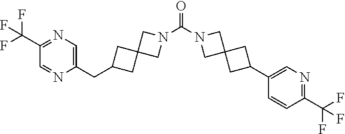 [SMILES: O=C(N1CC2(CC(Cc3cnc(C(F)(F)F)cn3)C2)C1)N1CC2(CC(c3ccc(C(F)(F)F)nc3)C2)C1] | [6-[[5- (trifluoromethyl)pyrazin- 2-yl]methyl]-2- azaspiro[3.3]heptan-2- yl]-[6-[6- (trifluoromethyl)-3- pyridyl]-2- azaspiro[3.3]heptan-2- yl]methanone | A.11 and D.4 | 526.2 [M + H]⁺ |
| 520 | 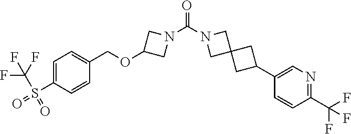 [SMILES: O=C(N1CC(OCc2ccc(S(=O)(=O)C(F)(F)F)cc2)C1)N1CC2(CC(c3ccc(C(F)(F)F)nc3)C2)C1] | [6-[6-(trifluoromethyl)- 3-pyridyl]-2- azaspiro[3.3]heptan-2- yl]-[3-[[4- (trifluoromethylsulfonyl) phenyl]methoxy]azetidin- 1-yl]methanone | A.11 and D.232 | 564.3 [M + H]⁺ |
| 523 | 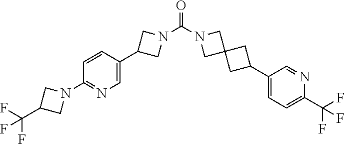 [SMILES: O=C(N1CC(c2ccc(N3CC(C(F)(F)F)C3)nc2)C1)N1CC2(CC(c3ccc(C(F)(F)F)nc3)C2)C1] | [3-[6-[3- (trifluoromethyl) azetidin- 1-yl]-3- pyridyl]azetidin-1-yl]- [6-[6-(trifluoromethyl)- 3-pyridyl]-2- azaspiro[3.3]heptan-2- yl]methanone | A.11 and D.183 | 526.3 [M + H]⁺ |
| 524 | 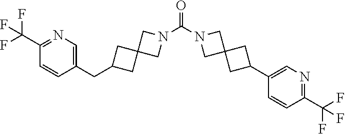 [SMILES: O=C(N1CC2(CC(Cc3ccc(C(F)(F)F)nc3)C2)C1)N1CC2(CC(c3ccc(C(F)(F)F)nc3)C2)C1] | [6-[6-(trifluoromethyl)- 3-pyridyl]-2- azaspiro[3.3]heptan-2- yl]-[6-[[6- (trifluoromethyl)-3- pyridyl]methyl]-2- azaspiro[3.3]heptan-2- yl]methanone | A.11 and D.3 | 525.3 [M + H]⁺ |
| 525 | 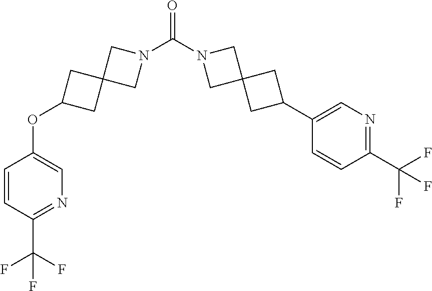 [SMILES: O=C(N1CC2(CC(Oc3ccc(C(F)(F)F)nc3)C2)C1)N1CC2(CC(c3ccc(C(F)(F)F)nc3)C2)C1] | [6-[6-(trifluoromethyl)- 3-pyridyl]-2- azaspiro[3.3]heptan-2- yl]-[6-[[6- (trifluoromethyl)-3- pyridyl]oxy]-2- azaspiro[3.3]heptan-2- yl]methanone | A.11 and B.28 | 527.3 [M + H]⁺ |
| 526 | 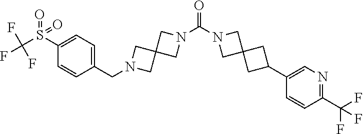 [SMILES: O=C(N1CC2(CC(c3ccc(C(F)(F)F)nc3)C2)C1)N1CC2(CN(Cc3ccc(S(=O)(=O)C(F)(F)F)cc3)C2)C1] | [6-[6-(trifluoromethyl)- 3-pyridyl]-2- azaspiro[3.3]heptan-2- yl]-[6-[[4- (trifluoromethylsulfonyl) phenyl]methyl]-2,6- diazaspiro[3.3]heptan- 2-yl]methanone | A.11 and D.54 | 589.4 [M + H]⁺ |
| 527 | 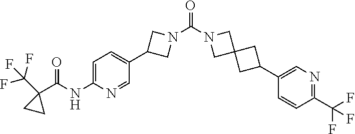 [SMILES: O=C(N1CC(c2ccc(NC(=O)C3(C(F)(F)F)CC3)nc2)C1)N1CC2(CC(c3ccc(C(F)(F)F)nc3)C2)C1] | [3-[6-[1- (trifluoromethyl) cyclopropyl] methylamino]-3- pyridyl]azetidin-1-yl]- [6-[6-(trifluoromethyl)- 3-pyridyl]-2- azaspiro[3.3]heptan-2- yl]methanone | A.11 and D.179 | 540.4 [M + H]⁺ |
| 528 | 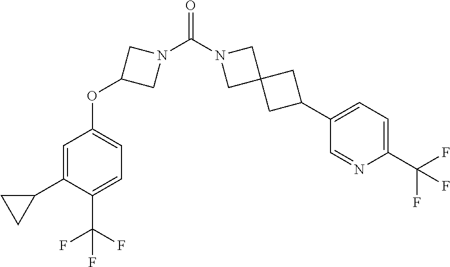 [SMILES: O=C(N1CC(Oc2ccc(C(F)(F)F)c(C3CC3)c2)C1)N1CC2(CC(c3ccc(C(F)(F)F)nc3)C2)C1] | [3-[3-cyclopropyl-4- (trifluoromethyl) phenoxylazetidin- 1-yl]-[6-[6- (trifluoromethyl)-3- pyridyl]-2- azaspiro[3.3]heptan-2- yl]methanone | A.11 and D.235 | 526.3 [M + H]⁺ |
| 529 | 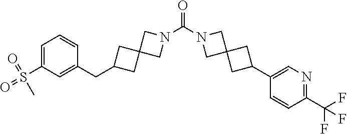 [SMILES: CS(=O)(=O)c1cccc(CC2CC3(C2)CN(C(=O)N2CC4(CC(c5ccc(C(F)(F)F)nc5)C4)C2)C3)c1] | [6-[(3- methylsulfonylphenyl) methyl]-2- azaspiro[3.3]heptan-2- yl]-[6-[6- (trifluoromethyl)-3- pyridyl]-2- azaspiro[3.3]heptan-2- yl]methanone | A.11 and D.8 | 534.3 [M + H]⁺ |
| 530 | 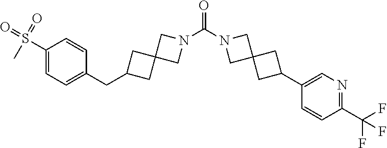 [SMILES: CS(=O)(=O)c1ccc(CC2CC3(C2)CN(C(=O)N2CC4(CC(c5ccc(C(F)(F)F)nc5)C4)C2)C3)cc1] | [6-[(4- methylsulfonylphenyl) methyl]-2- azaspiro[3.3]heptan-2- yl]-[6-[6- (trifluoromethyl)-3- pyridyl]-2- azaspiro[3.3]heptan-2- yl]methanone | A.11 and D.7 | 534.3 [M + H]⁺ |
| 531 | 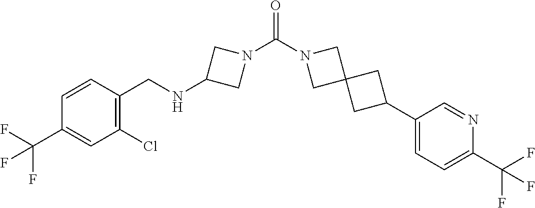 [SMILES: O=C(N1CC(NCc2ccc(C(F)(F)F)cc2Cl)C1)N1CC2(CC(c3ccc(C(F)(F)F)nc3)C2)C1] | [3-[[2-chloro-4- (trifluoromethyl)phenyl \|methylamino]azetidin- 1-yl]-[6-[6- (trifluoromethyl)-3- pyridyl]-2- azaspiro[3.3]heptan-2- yl]methanone | A.11 and D.130 | 533.3 [M + H]⁺ |
| 532 | 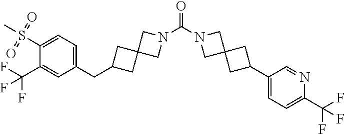 [SMILES: CS(=O)(=O)c1ccc(CC2CC3(C2)CN(C(=O)N2CC4(CC(c5ccc(C(F)(F)F)nc5)C4)C2)C3)cc1C(F)(F)F] | [6-[[4-methylsulfonyl- 3- (trifluoromethyl)phenyl] methyl]-2- azaspiro[3.3]heptan-2- yl]-[6-[6- (trifluoromethyl)-3- pyridyl]-2- azaspiro[3.3]heptan-2- | A.11 and D.24 | 602.4 [M + H]⁺ |
| | | yl]methanone | | |
| 533 | 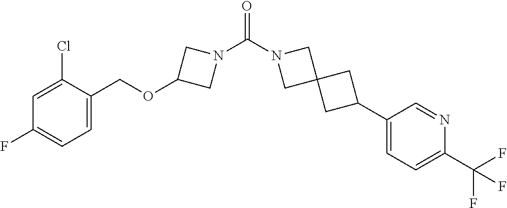 [SMILES: O=C(N1CC(OCc2ccc(F)cc2Cl)C1)N1CC2(CC(c3ccc(C(F)(F)F)nc3)C2)C1] | [3-[(2-chloro-4-fluoro- phenyl)methoxy] azetidin-1-yl]-[6-[6- (trifluoromethyl)-3- pyridyl]-2- azaspiro[3.3]heptan-2- yl]methanone | A.11 and B.25 | 484.2 [M + H]⁺ |
| 534 | 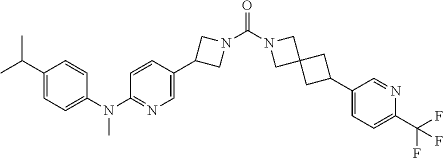 [SMILES: CC(C)c1ccc(N(C)c2ccc(C3CN(C(=O)N4CC5(CC(c6ccc(C(F)(F)F)nc6)C5)C4)C3)cn2)cc1] | [3-[6-(4-isopropyl-N- methyl-anilino)-3- pyridyl]azetidin-1-yl]- [6-[6-(trifluoromethyl)- 3-pyridyl]-2- azaspiro[3.3]heptan-2- yl]methanone | A.11 and D.291 | 550.5 [M + H]⁺ |
| 535 | 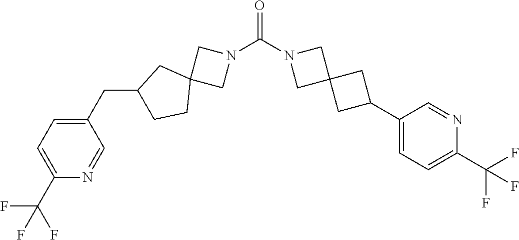 [SMILES: O=C(N1CC2(CCC(Cc3ccc(C(F)(F)F)nc3)C2)C1)N1CC2(CC(c3ccc(C(F)(F)F)nc3)C2)C1] | [6-[6-(trifluoromethyl)- 3-pyridyl]-2- azaspiro[3.3]heptan-2- yl]-[6-[[6- (trifluoromethyl)-3- pyridyl]methyl]-2- azaspiro[3.4]octan-2- yl]methanone | A.11 and D.95 | 539.4 [M + H]⁺ |
| 536 | 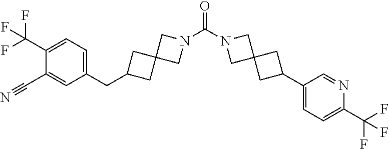 [SMILES: N#Cc1cc(CC2CC3(C2)CN(C(=O)N2CC4(CC(c5ccc(C(F)(F)F)nc5)C4)C2)C3)ccc1C(F)(F)F] | 2-(trifluoromethyl)-5- [[2-[6-[6- (trifluoromethyl)-3- pyridyl]-2- azaspiro[3.3]heptane-2- carbonyl]-2- azaspiro[3.3]heptan-6- yl]methyl]benzonitrile | A.11 and D.13 | 549.4 [M + H]⁺ |

### Example 214 and Example 216

### [6-(3-cyclopropyl-1,2,4-triazol-1-yl)-2-azaspiro[3.3]heptan-2-yl-[3-[6-[(3R)-3-hydroxy-3-(trifluoromethyl)pyrrolidin-1-yl]—3-pyridyl]azetidin-1-yl]methanone (Example 214) and [6-(3-cyclopropyl-1,2,4-triazol-1-yl)-2-azaspiro[3.3]heptan-2-yll-[3-[6-[(3S)-3-hydroxy-3-(trifluoromethyl)pyrrolidinol-3-pyridyl]azetidin-1-yl]methanone (Example 216)

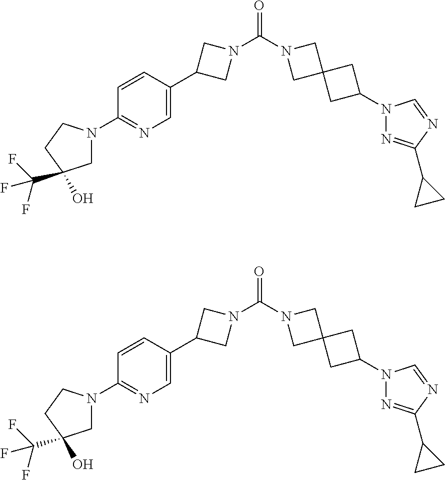 [SMILES: O=C(N1CC(c2ccc(N3CC[C@](O)(C(F)(F)F)C3)nc2)C1)N1CC2(CC(n3cnc(C4CC4)n3)C2)C1 ; O=C(N1CC(c2ccc(N3CC[C@@](O)(C(F)(F)F)C3)nc2)C1)N1CC2(CC(n3cnc(C4CC4)n3)C2)C1]

Example 212 (120 mg, 0.23 mmol) was purified by chiral SFC, to give Example 214 (58.8 mg, 27% yield) and Example 216 (55.7 mg, 27.1% yield). The stereochemistry was arbitrarily assigned. MS (ESI): m/z=518.3 [M+H]⁺ (for both enantiomers)

### Example 239

### [6-(3-cyclopropyl-1,2,4-triazol-1-yl)-2-azaspiro[3.3]heptan-2-yl-[2-(2,2-dimethylpropylsulfonyl)-2,6-diazaspiro[3.3]heptan-6-yl]methanone

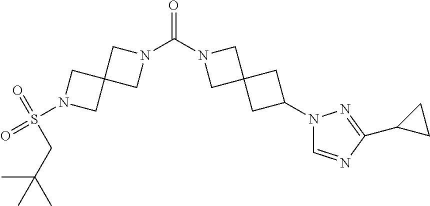 [SMILES: CC(C)(C)CS(=O)(=O)N1CC2(CN(C(=O)N3CC4(CC(n5cnc(C6CC6)n5)C4)C3)C2)C1]

To a solution of [6-(3-cyclopropyl-1,2,4-triazol-1-yl)-2-azaspiro[3.3]heptan-2-yl]-(2,6-diazaspiro[3.3]heptan-2-yl)methanone;2,2,2-trifluoroacetic acid (150 mg, 0.189 mmol) in dichloromethane (1.5 mL) were added DIPEA (164.3 μL, 0.943 mmol) and 2,2-dimethylpropane-1-sulfonyl chloride (33.8 mg, 0.198 mmol). The mixture was stirred for 18 h at 23° C., before being evaporated. Purification by RP-HPLC gave the title compound (29.4 mg, 33%). MS (ESI): m/z=463.4 [M+H]⁺

### Step a): (6-(3-cyclopropyl-]H-1, 2,4-triazol-1-yl)-2-azaspiro[3.3]heptan-2-yl)(]H-1, 2,4-triazol-1-yl)methanone

To a suspension of 6-(3-cyclopropyl-1H-1,2,4-triazol-1-yl)-2-azaspiro[3.3]heptane 2,2,2-trifluoroacetate (A.1; 18 g, 56.6 mmol) in dry CH₂Cl₂ (240 ml) cooled down to 0° C. was added DIPEA (29.6 mL, 170 mmol) (white suspension) followed by addition of CDT (9.75 g, 59.4 mmol). The mixture was stirred for 5 min at 0° C. for 5 min and for 45 min at 23° C., before being diluted with DCM. The mixture was extracted with 1 M aqueous Na₂CO₃. The combined organic layers were dried over Na₂SO₄, filtered, and evaporated, to give the title compound (16.59 g, 98% yield). MS (ESI): m/z=300.2 [M+H]⁺

### Step b): 6-[6-(3-cyclopropyl-1, 2,4-triazol-1-yl)-2-azaspiro[3.3]heptane-2-carbonyl]-2,6-diazaspiro[3.3]heptane-2-carboxylic acid tert-butyl ester

To a solution of [6-(3-cyclopropyl-1,2,4-triazol-1-yl)-2-azaspiro[3.3]heptan-2-yl]-(1,2,4-triazol-1-yl)methanone (4 g, 13.36 mmol) in DMF (50 mL) were added 2,6-diazaspiro[3.3]heptane-2-carboxylic acid tert-butyl ester;oxalic acid (3.38 g, 6.95 mmol) and DIPEA (4.67 mL, 26.73 mmol), at 23° C. The mixture was heated to 100° C. and stirred for 18 h at this temperature, before being evaporated. The residue was partitioned between EtOAc and 1 M aqueous Na₂CO₃. The organic layer was collected, and the aqueous layer was back-extracted with EtOAc. The combined organic layers were dried over Na₂SO₄ and evaporated. Purification by FC(SiO₂; DCM/MeOH) gave the title compound (5.23 g, 86.80%). MS (ESI): m/z=429.4 [M+H]⁺

### Step c): [6-(3-cyclopropyl-, 2, 4-triazol-4 Ay-2-azaspiro[3.3]heptan-2yl]-(2, 6-diazaspiro[3.3]heptan-2yl)methanone 0.1:2 2,2,2-trifluoroacetic acid

To a solution of tert-butyl 6-(6-(3-cyclopropyl-1H-1,2,4-triazol-1-yl)-2-azaspiro[3.3]heptane-2-carbonyl)-2,6-diazaspiro[3.3]heptane-2-carboxylate (2.53 g, 5.91 mmol) in dichloromethane (12 mL) was added TFA (4.55 mL, 59.06 mmol). The mixture was stirred for 18 h at 23° C. before being evaporated to give the title compound (4.65 g, 99.0%) as an oil. MS (ESI): m/z=329.3 [M+H]⁺

In analogy to Example 239, Examples in the following table were generated using the respective building blocks AX and the corresponding commercially available sulfonyl chlorides.

| | | | | MS, |
|---|---|---|---|---|
| | | | Building | ESI: |
| Ex. | Structure | Systematic Name | Blocks | m/z |
| 233 | 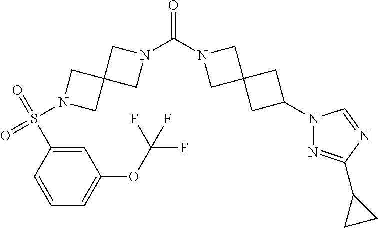 [SMILES: O=C(N1CC2(CC(n3cnc(C4CC4)n3)C2)C1)N1CC2(C1)CN(S(=O)(=O)c1cccc(OC(F)(F)F)c1)C2] | [6-(3-cyclopropyl-1,2,4- triazol-1-yl)-2- azaspiro[3.3]heptan-2- yl]-[2-[3- (trifluoromethoxy)phenyl] sulfonyl-2,6- diazaspiro[3.3]heptan-6- yl]methanone | A.1 and 3- (trifluoro methoxy) benzene- sulfonyl chloride (CAS: 220227- 84-9) | 553.4 [M + H]⁺ |
| 230 | 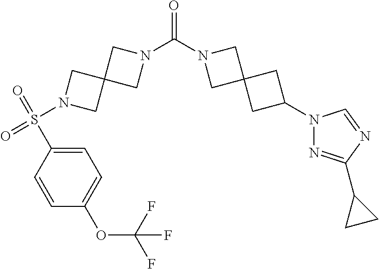 [SMILES: O=C(N1CC2(CC(n3cnc(C4CC4)n3)C2)C1)N1CC2(C1)CN(S(=O)(=O)c1ccc(OC(F)(F)F)cc1)C2] | [6-(3-cyclopropyl-1,2,4- triazol-1-yl)-2- azaspiro[3.3]heptan-2- yl]-[2-[4- (trifluoromethoxy)phenyl] sulfonyl-2,6- diazaspiro[3.3]heptan-6- yl]methanone | A.1 and 4- (trifluoro methoxy) benzene- sulfonyl chloride (CAS: 94108-56- 2) | 553.4 [M + H]⁺ |
| 223 | 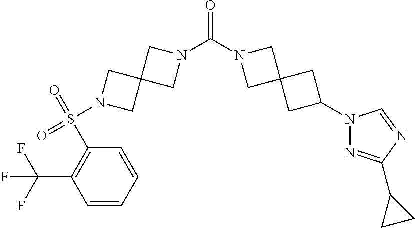 [SMILES: O=C(N1CC2(CC(n3cnc(C4CC4)n3)C2)C1)N1CC2(C1)CN(S(=O)(=O)c1ccccc1C(F)(F)F)C2] | [6-(3-cyclopropyl-1,2,4- triazol-1-yl)-2- azaspiro[3.3]heptan-2- yl]-[2-[2- (trifluoromethyl)phenyl] sulfonyl-2,6- diazaspiro[3.3]heptan-6- yl]methanone | A.1 and 2- (trifluoro methyl) benzene- sulfonyl chloride (CAS: 776-04-5) | 537.4 [M + H]⁺ |
| 209 | 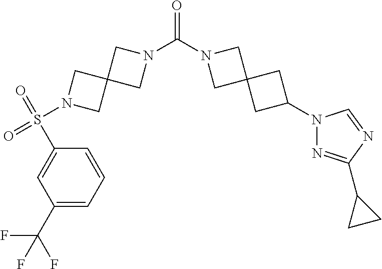 [SMILES: O=C(N1CC2(CC(n3cnc(C4CC4)n3)C2)C1)N1CC2(C1)CN(S(=O)(=O)c1cccc(C(F)(F)F)c1)C2] | [6-(3-cyclopropyl-1,2,4- triazol-1-yl)-2- azaspiro[3.3]heptan-2- yl]-[2-[3- (trifluoromethyl)phenyl] sulfonyl-2,6- diazaspiro[3.3]heptan-6- yl]methanone | A.1 and 3- (trifluoro methyl) benzene- sulfonyl chloride (CAS: 777-44-6) | 537.3 [M + H]⁺ |
| 225 | 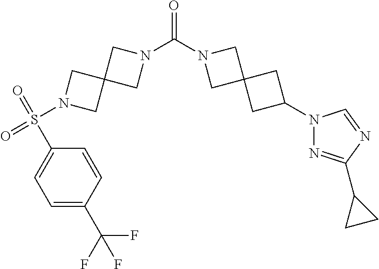 [SMILES: O=C(N1CC2(CC(n3cnc(C4CC4)n3)C2)C1)N1CC2(C1)CN(S(=O)(=O)c1ccc(C(F)(F)F)cc1)C2] | [6-(3-cyclopropyl-1,2,4- triazol-1-yl)-2- azaspiro[3.3]heptan-2- yl]-[2-[4- (trifluoromethyl)phenyl] sulfonyl-2,6- diazaspiro[3.3]heptan-6- yl]methanone | A.1 and 4- (trifluoro methyl) benzene-1- sulfonyl chloride (CAS: 2991-42- 6) | 537.3 [M + H]⁺ |
| 228 | 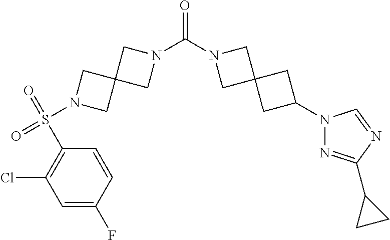 [SMILES: O=C(N1CC2(CC(n3cnc(C4CC4)n3)C2)C1)N1CC2(C1)CN(S(=O)(=O)c1ccc(F)cc1Cl)C2] | [2-(2-chloro-4-fluoro- phenyl)sulfonyl-2,6- diazaspiro[3.3]heptan-6- yl]-[6-(3-cyclopropyl- 1,2,4-triazol-1-yl)-2- azaspiro[3.3]heptan-2- yl]methanone | A.1 and 2- chloro-4- fluoro- benzene- sulfonyl chloride (CAS: 85958-57- 2) | 521.2 [M + H]⁺ |
| 231 | 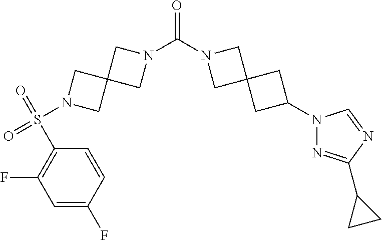 [SMILES: O=C(N1CC2(CC(n3cnc(C4CC4)n3)C2)C1)N1CC2(C1)CN(S(=O)(=O)c1ccc(F)cc1F)C2] | [6-(3-cyclopropyl-1,2,4- triazol-1-yl)-2- azaspiro[3.3]heptan-2- yl]-[2-(2,4- difluorophenyl)sulfonyl- 2,6- diazaspiro[3.3]heptan-6- yl]methanone | A.1 and 2,4- difluoro- benzene-1- sulfonyl chloride (CAS: 13918-92- 8) | 505.3 [M + H]⁺ |
| 251 | 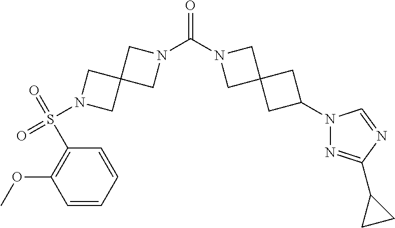 [SMILES: COc1ccccc1S(=O)(=O)N1CC2(CN(C(=O)N3CC4(CC(n5cnc(C6CC6)n5)C4)C3)C2)C1] | [6-(3-cyclopropyl-1,2,4- triazol-1-yl)-2- azaspiro[3.3]heptan-2- yl]-[2-(2- methoxyphenyl)sulfonyl- 2,6- diazaspiro[3.3]heptan-6- yl]methanone | A.1 and 2- methoxy- benzene- sulfonyl chloride (CAS: 10130-87- 7) | 499.3 [M + H]⁺ |

### Example 248

### [6-(3-cyclopropyl-1,2,4-triazol-1-yl)-2-azaspiro[3.3]heptan-2-yl]−[3-[6-(trifluoromethyl)pyridazin-3-yl]oxyazetidin-1-yl]methanone

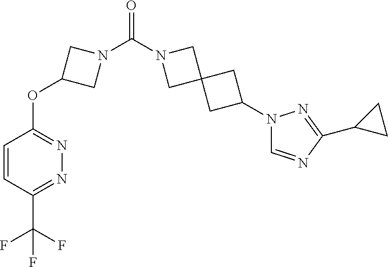 [SMILES: O=C(N1CC(Oc2ccc(C(F)(F)F)nn2)C1)N1CC2(CC(n3cnc(C4CC4)n3)C2)C1]

To a solution of (6-(3-cyclopropyl-1H-1,2,4-triazol-1-yl)-2-azaspiro[3.3]heptan-2-yl)(3-hydroxyazetidin-1-yl)methanone (90 mg, 297 μmol) in dry DMF (1.69 mL) under Ar was added NaH (13.1 mg, 326 μmol), and the mixture was stirred for 10 min at 23° C. followed by addition of 3-chloro-6-(trifluoromethyl)pyridazine (67.7 mg, 371 μmol). The mixture was stirred for 18 h at 90° C., before being cooled down. Purification by RP-HPLC gave the title compound (70.3 mg, 51.7% yield). MS (ESI): m/z=450.2 [M+H]⁺

### Step a): (6-(3-cyclopropyl-]H-1, 2,4-triazol-1-yl)-2-azaspiro[3.3]heptan-2-yl)(]H-1, 2,4-triazol-1-yl)methanone

To a suspension of 6-(3-cyclopropyl-1H-1,2,4-triazol-1-yl)-2-azaspiro[3.3]heptane 2,2,2-trifluoroacetate (18 g, 56.6 mmol) in dry DCM (240 mL) cooled down to 0° C. was added DIPEA (29.6 mL, 170 mmol) (white suspension) followed by addition of di(1H-1,2,4-triazol-1-yl)methanone (9.75 g, 59.4 mmol). The mixture was stirred for 5 min at 0° C. and for another 45 min at 23° C., before being diluted with DCM. The organic layer was extracted with 1 M aqueous Na₂CO₃, dried over Na₂SO₄, filtered, and evaporated, to give the title compound (16.6 g, 98% yield). MS (ESI): m/z=300.2 [M+H]⁺

### Step b): 6-(3-cyclopropyl-]H-1,2,4-triazol-1-yl)-2-azaspiro[3.3]heptan-2-yl)(3-hydroxyazetidin-1-yl)methanone

To a solution of (6-(3-cyclopropyl-1H-1,2,4-triazol-1-yl)-2-azaspiro[3.3]heptan-2-yl)(1H-1,2,4-triazol-1-yl)methanone (1.5 g, 5.01 mmol) in dry DMF (17 mL) was added azetidin-3-ol (458 mg, 6.26 mmol) and DIPEA (3.06 mL, 17.5 mmol) after which the reaction mixture was stirred for 18 h at 90° C., before being evaporated. The residue was partitioned between EtOAc and 2 M aqueous Na₂C₀₃. The organic layer was collected and the aqueous layer was back-extracted with EtOAc. The combined organic layers were dried over Na₂SO₄, filtered and evaporated, to give the title compound (1.48 g, 87.6 yield). MS (ESI): m/z=304.2 [M+H]⁺

### Example 291 and Example 301

### (2S)-2-[1-[6-(3-cyclopropyl-1,2,4-triazol-1-yl)-2-azaspiro[3.3]heptane-2-carbonyl]-4-piperidyl]-2-(4-fluorophenyl)acetamide (Example 291) (2R)-2-[1-[6-(3-cyclopropyl-1,2,4-triazol-1-yl)-2-azaspiro[3.3]heptane-2-carbonyl]-4-piperidyl]-2-(4-fluorophenyl)acetamide Example 301

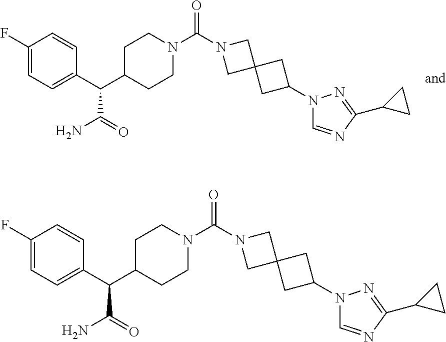 [SMILES: NC(=O)[C@H](c1ccc(F)cc1)C1CCN(C(=O)N2CC3(CC(n4cnc(C5CC5)n4)C3)C2)CC1 ; NC(=O)[C@@H](c1ccc(F)cc1)C1CCN(C(=O)N2CC3(CC(n4cnc(C5CC5)n4)C3)C2)CC1]

Example 305 (83.4 mg, 0.17 mmol) was purified by chiral SFC, to give Example 291 (33.4 mg, 21% yield) and Example 301 (21.8 mg, 13.7% yield). Arbitrary assignment of the stereochemistry. MS (ESI): m/z=467.5 [M+H]⁺

### Example 493 and Example 494

### (2R)-1-[4-[1-[6-(3-cyclopropyl-1,2,4-triazol-1-yl)-2-azaspiro[3.3]heptane-2-carbonyl]azetidin-3-yl]phenyl]-4,4-difluoro-piperidine-2-carboxamide (Example 493) and (2R)-1-[4-[1-[6-(3-cyclopropyl-1,2,4-triazol-1-yl)-2-azaspiro[3.3]heptane-2-carbonyl]azetidin-3-yl]phenyl]-4,4-difluoro-piperidine-2-carboxamide (Example 494)

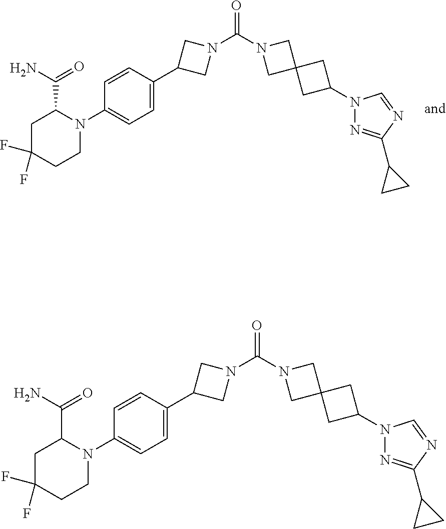 [SMILES: NC(=O)[C@H]1CC(F)(F)CCN1c1ccc(C2CN(C(=O)N3CC4(CC(n5cnc(C6CC6)n5)C4)C3)C2)cc1 ; NC(=O)C1CC(F)(F)CCN1c1ccc(C2CN(C(=O)N3CC4(CC(n5cnc(C6CC6)n5)C4)C3)C2)cc1]

Example 503 (56.0 mg, 0.101 mmol) was purified by chiral SFC, to give Example 493 (19.8 mg, 35.4%) and Example 494 (20.4 mg, 36.4%). MS (ESI): m/z=526.3 [M+H]⁺

The following Examples can be made in analogy to the procedures already discussed, or using literature techniques:

| | | | MS, |
|---|---|---|---|
| Ex. | Structure | Systematic Name | ESI: m/z |
| 184 | 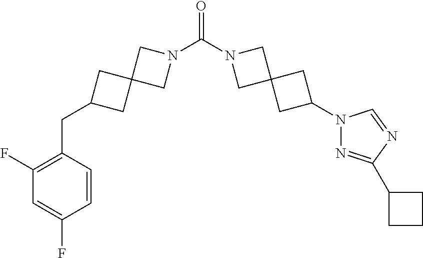 [SMILES: O=C(N1CC2(CC(Cc3ccc(F)cc3F)C2)C1)N1CC2(CC(n3cnc(C4CCC4)n3)C2)C1] | [6-(3-cyclobutyl-1,2,4- triazol-1-yl)-2- azaspiro[3.3]heptan-2-yl]- [6-[(2,4- difluorophenyl)methyl]-2- azaspiro[3.3]heptan-2- yl]methanone | 468.3 [M + H]⁺ |
| 185 | 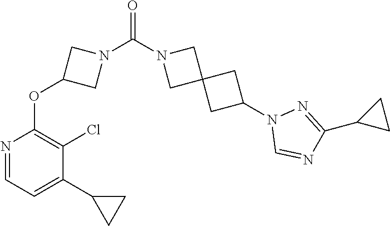 [SMILES: O=C(N1CC(Oc2nccc(C3CC3)c2Cl)C1)N1CC2(CC(n3cnc(C4CC4)n3)C2)C1] | [3-[(3-chloro-4-cyclopropyl- 2-pyridyl)oxy]azetidin-1- yl]-[6-(3-cyclopropyl-1,2,4- triazol-1-yl)-2- azaspiro[3.3]heptan-2- yl]methanone | 455.3 [M + H]⁺ |
| 186 | 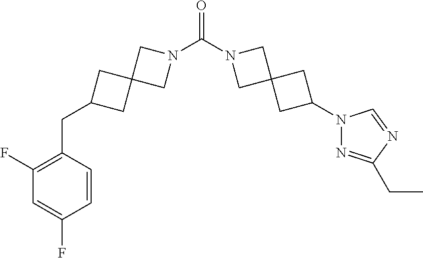 [SMILES: CCc1ncn(C2CC3(C2)CN(C(=O)N2CC4(CC(Cc5ccc(F)cc5F)C4)C2)C3)n1] | [6-[(2,4- difluorophenyl)methyl]-2- azaspiro[3.3]heptan-2-yl]- [6-(3-ethyl-1,2,4-triazol-1- yl)-2-azaspiro[3.3]heptan-2- yl]methanone | 442.3 [M + H]⁺ |
| 194 | 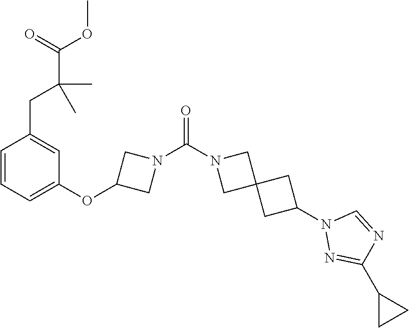 [SMILES: COC(=O)C(C)(C)Cc1cccc(OC2CN(C(=O)N3CC4(CC(n5cnc(C6CC6)n5)C4)C3)C2)c1] | methyl 3-[3-[1-[6-(3- cyclopropyl-1,2,4-triazol-1- yl)-2-azaspiro[3.3]heptane- 2-carbonyl]azetidin-3- yl]oxyphenyl]-2,2-dimethyl- propanoate | 494.3 [M + H]⁺ |
| 197 | 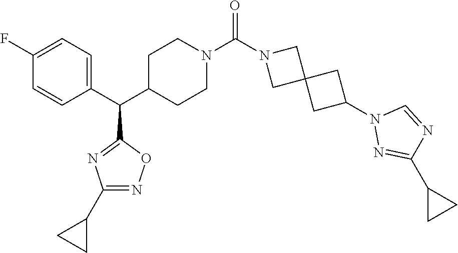 [SMILES: O=C(N1CCC([C@H](c2ccc(F)cc2)c2nc(C3CC3)no2)CC1)N1CC2(CC(n3cnc(C4CC4)n3)C2)C1] | [4-[(R)-(3-cyclopropyl- 1,2,4-oxadiazol-5-yl)-(4- fluorophenyl)methyl]-1- piperidyl]-[6-(3- cyclopropyl-1,2,4-triazol-1- yl)-2-azaspiro[3.3]heptan-2- yl]methanone | 532.4 [M + H]⁺ |
| 203 | 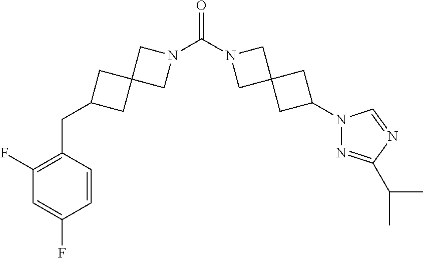 [SMILES: CC(C)c1ncn(C2CC3(C2)CN(C(=O)N2CC4(CC(Cc5ccc(F)cc5F)C4)C2)C3)n1] | [6-[(2,4- difluorophenyl)methyl]-2- azaspiro[3.3]heptan-2-yl]- [6-(3-isopropyl-1,2,4- triazol-1-yl)-2- azaspiro[3.3]heptan-2- yl]methanone | 456.3 [M + H]⁺ |
| 204 | 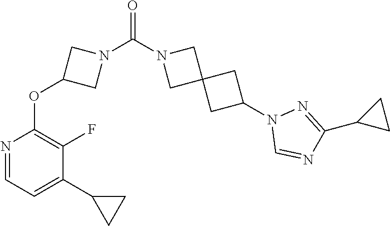 [SMILES: O=C(N1CC(Oc2nccc(C3CC3)c2F)C1)N1CC2(CC(n3cnc(C4CC4)n3)C2)C1] | [3-[(4-cyclopropyl-3-fluoro- 2-pyridyl)oxy]azetidin-1- yl]-[6-(3-cyclopropyl-1,2,4- triazol-1-yl)-2- azaspiro[3.3]heptan-2- yl]methanone | 439.3 [M + H]⁺ |
| 207 | 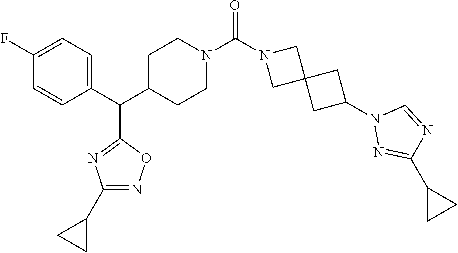 [SMILES: O=C(N1CCC(C(c2ccc(F)cc2)c2nc(C3CC3)no2)CC1)N1CC2(CC(n3cnc(C4CC4)n3)C2)C1] | [4-[(3-cyclopropyl-1,2,4- oxadiazol-5-yl)-(4- fluorophenyl)methyl]-1- piperidyl]-[6-(3- cyclopropyl-1,2,4-triazol-1- yl)-2-azaspiro[3.3]heptan-2- yl]methanone | 532.3 [M + H]⁺ |
| 208 | 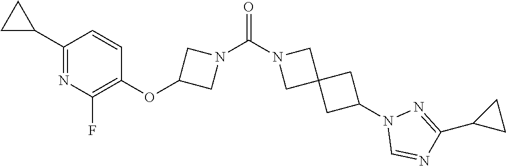 [SMILES: O=C(N1CC(Oc2ccc(C3CC3)nc2F)C1)N1CC2(CC(n3cnc(C4CC4)n3)C2)C1] | [3-[(6-cyclopropyl-2-fluoro- 3-pyridyl)oxy]azetidin-1- yl]-[6-(3-cyclopropyl-1,2,4- triazol-1-yl)-2- azaspiro[3.3]heptan-2- yl]methanone | 439.3 [M + H]⁺ |
| 210 | 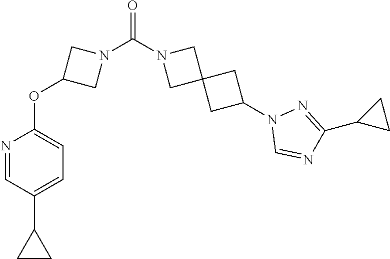 [SMILES: O=C(N1CC(Oc2ccc(C3CC3)cn2)C1)N1CC2(CC(n3cnc(C4CC4)n3)C2)C1] | [3-[(5-cyclopropyl-2- pyridyl)oxy]azetidin-1-yl]- [6-(3-cyclopropyl-1,2,4- triazol-1-yl)-2- azaspiro[3.3]heptan-2- yl]methanone | 421.4 [M + H]⁺ |
| 213 | 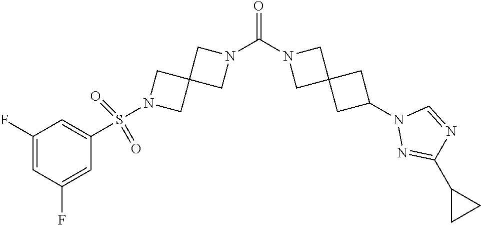 [SMILES: O=C(N1CC2(CC(n3cnc(C4CC4)n3)C2)C1)N1CC2(C1)CN(S(=O)(=O)c1cc(F)cc(F)c1)C2] | [6-(3-cyclopropyl-1,2,4- triazol-1-yl)-2- azaspiro[3.3]heptan-2-yl]- [2-(3,5- difluorophenyl)sulfonyl-2,6- diazaspiro[3.3]heptan-6- yl]methanone | 505.3 [M + H]⁺ |
| 215 | 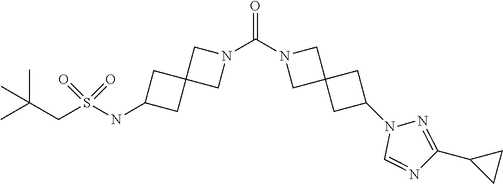 [SMILES: CC(C)(C)CS(=O)(=O)NC1CC2(C1)CN(C(=O)N1CC3(CC(n4cnc(C5CC5)n4)C3)C1)C2] | N-[2-[6-(3-cyclopropyl- 1,2,4-triazol-1-yl)-2- azaspiro[3.3]heptane-2- carbonyl]-2- azaspiro[3.3]heptan-6-yl]- 2,2-dimethyl-propane-1- sulfonamide | 477.3 [M + H]⁺ |
| 218 | 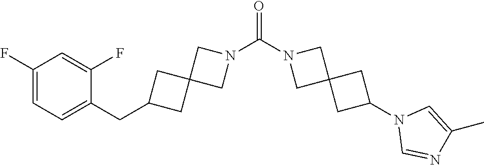 [SMILES: Cc1cn(C2CC3(C2)CN(C(=O)N2CC4(CC(Cc5ccc(F)cc5F)C4)C2)C3)cn1] | [6-[(2,4- difluorophenyl)methyl]-2- azaspiro[3.3]heptan-2-yl]- [6-(4-methylimidazol-1-yl)- 2-azaspiro[3.3]heptan-2- yl]methanone | 427.3 [M + H]⁺ |
| 224 | 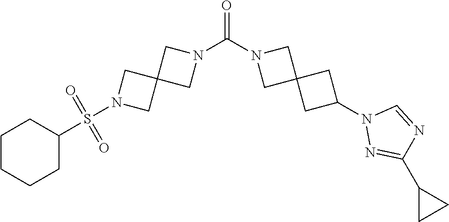 [SMILES: O=C(N1CC2(CC(n3cnc(C4CC4)n3)C2)C1)N1CC2(C1)CN(S(=O)(=O)C1CCCCC1)C2] | (2-cyclohexylsulfonyl-2,6- diazaspiro[3.3]heptan-6-yl)- [6-(3-cyclopropyl-1,2,4- triazol-1-yl)-2- azaspiro[3.3]heptan-2- yl]methanone | 475.4 [M + H]⁺ |
| 227 | 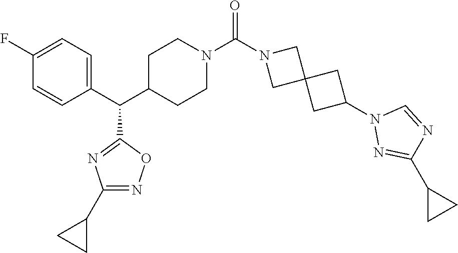 [SMILES: O=C(N1CCC([C@@H](c2ccc(F)cc2)c2nc(C3CC3)no2)CC1)N1CC2(CC(n3cnc(C4CC4)n3)C2)C1] | [4-[(S)-(3-cyclopropyl- 1,2,4-oxadiazol-5-yl)-(4- fluorophenyl)methyl]-1- piperidyl]-[6-(3- cyclopropyl-1,2,4-triazol-1- yl)-2-azaspiro[3.3]heptan-2- yl]methanone | 532.4 [M + H]⁺ |
| 235 | 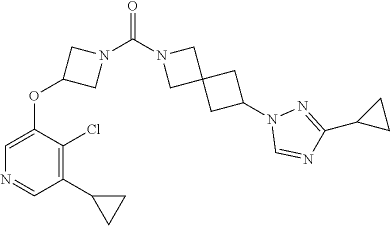 [SMILES: O=C(N1CC(Oc2cncc(C3CC3)c2Cl)C1)N1CC2(CC(n3cnc(C4CC4)n3)C2)C1] | [3-[(4-chloro-5-cyclopropyl- 3-pyridyl)oxy]azetidin-1- yl]-[6-(3-cyclopropyl-1,2,4- triazol-1-yl)-2- azaspiro[3.3]heptan-2- yl]methanone | 455.4 [M + H]⁺ |
| 237 | 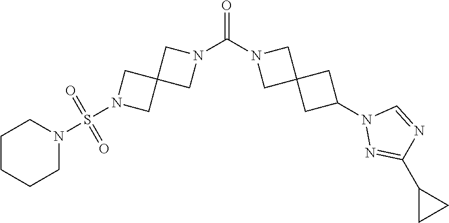 [SMILES: O=C(N1CC2(CC(n3cnc(C4CC4)n3)C2)C1)N1CC2(C1)CN(S(=O)(=O)N1CCCCC1)C2] | [6-(3-cyclopropyl-1,2,4- triazol-1-yl)-2- azaspiro[3.3]heptan-2-yl]- [2-(1-piperidylsulfonyl)-2,6- diazaspiro[3.3]heptan-6- yl]methanone | 476.4 [M + H]⁺ |
| 240 | 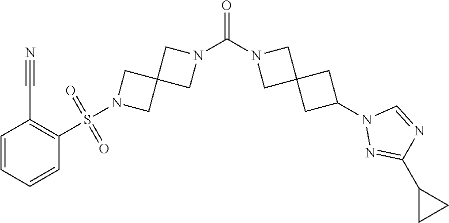 [SMILES: N#Cc1ccccc1S(=O)(=O)N1CC2(CN(C(=O)N3CC4(CC(n5cnc(C6CC6)n5)C4)C3)C2)C1] | 2-[6-[6-(3-cyclopropyl- 1,2,4-triazol-1-yl)-2- azaspiro[3.3]heptane-2- carbonyl]-2,6- diazaspiro[3.3]heptan-2- yl]sulfonyl]benzonitrile | 494.3 [M + H]⁺ |
| 242 | 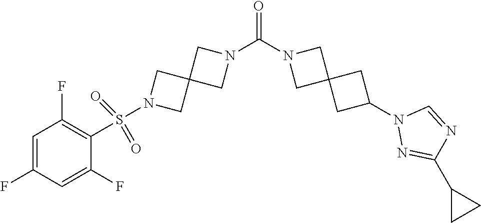 [SMILES: O=C(N1CC2(CC(n3cnc(C4CC4)n3)C2)C1)N1CC2(C1)CN(S(=O)(=O)c1c(F)cc(F)cc1F)C2] | [6-(3-cyclopropyl-1,2,4- triazol-1-yl)-2- azaspiro[3.3]heptan-2-yl]- [2-(2,4,6- trifluorophenyl)sulfonyl- 2,6-diazaspiro[3.3]heptan-6- yl]methanone | 523.2 [M + H]⁺ |
| 243 | 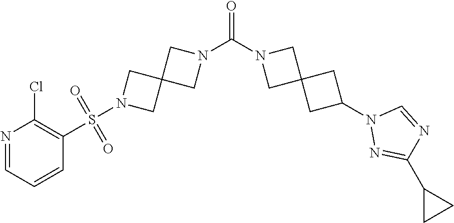 [SMILES: O=C(N1CC2(CC(n3cnc(C4CC4)n3)C2)C1)N1CC2(C1)CN(S(=O)(=O)c1cccnc1Cl)C2] | [2-[(2-chloro-3- pyridyl)sulfonyl]-2,6- diazaspiro[3.3]heptan-6-yl]- [6-(3-cyclopropyl-1,2,4- triazol-1-yl)-2- azaspiro[3.3]heptan-2- yl]methanone | 504.3 [M + H]⁺ |
| 244 | 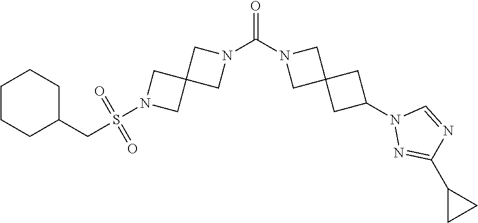 [SMILES: O=C(N1CC2(CC(n3cnc(C4CC4)n3)C2)C1)N1CC2(C1)CN(S(=O)(=O)CC1CCCCC1)C2] | [2- (cyclohexylmethylsulfonyl)- 2,6-diazaspiro[3.3]heptan-6- yl]-[6-(3-cyclopropyl-1,2,4- triazol-1-yl)-2- azaspiro[3.3]heptan-2- yl]methanone | 489.5 [M + H]⁺ |
| 245 | 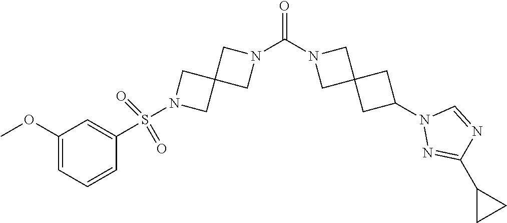 [SMILES: COc1cccc(S(=O)(=O)N2CC3(CN(C(=O)N4CC5(CC(n6cnc(C7CC7)n6)C5)C4)C3)C2)c1] | [6-(3-cyclopropyl-1,2,4- triazol-1-yl)-2- azaspiro[3.3]heptan-2-yl]- [2-(3- methoxyphenyl)sulfonyl- 2,6-diazaspiro[3.3]heptan-6- yl]methanone | 499.4 [M + H]⁺ |
| 246 | 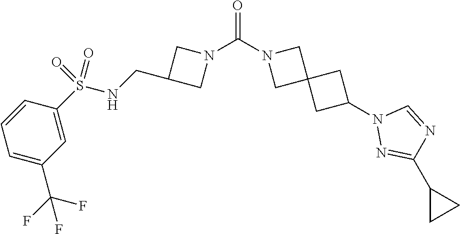 [SMILES: O=C(N1CC(CNS(=O)(=O)c2cccc(C(F)(F)F)c2)C1)N1CC2(CC(n3cnc(C4CC4)n3)C2)C1] | N-[[1-[6-(3-cyclopropyl- 1,2,4-triazol-1-yl)-2- azaspiro[3.3]heptane-2- carbonyl]azetidin-3- yl]methyl]-3- (trifluoromethyl)benzenesul fonamide | 525.4 [M + H]⁺ |
| 249 | 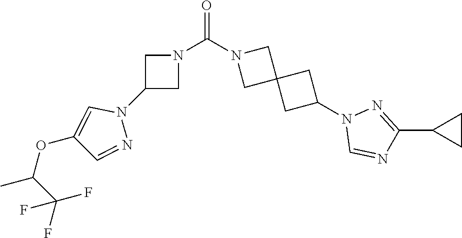 [SMILES: CC(Oc1cnn(C2CN(C(=O)N3CC4(CC(n5cnc(C6CC6)n5)C4)C3)C2)c1)C(F)(F)F] | [6-(3-cyclopropyl-1,2,4- triazol-1-yl)-2- azaspiro[3.3]heptan-2-yl]- [3-[4-(2,2,2-trifluoro-1- methyl-ethoxy)pyrazol-1- yl]azetidin-1-yl]methanone | 466.3 [M + H]⁺ |
| 250 | 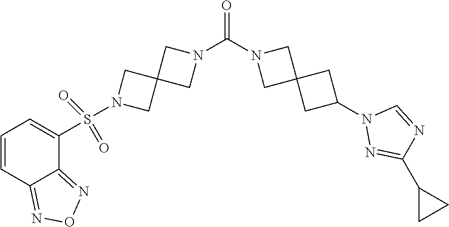 [SMILES: O=C(N1CC2(CC(n3cnc(C4CC4)n3)C2)C1)N1CC2(C1)CN(S(=O)(=O)c1cccc3nonc13)C2] | [2-(2,1,3-benzoxadiazol-4- ylsulfonyl)-2,6- diazaspiro[3.3]heptan-6-yl]- [6-(3-cyclopropyl-1,2,4- triazol-1-yl)-2- azaspiro[3.3]heptan-2- yl]methanone | 511.3 [M + H]⁺ |
| 255 | 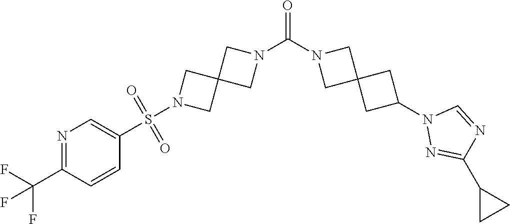 [SMILES: O=C(N1CC2(CC(n3cnc(C4CC4)n3)C2)C1)N1CC2(C1)CN(S(=O)(=O)c1ccc(C(F)(F)F)nc1)C2] | [6-(3-cyclopropyl-1,2,4- triazol-1-yl)-2- azaspiro[3.3]heptan-2-yl]- [2-[[6-(trifluoromethyl)-3- pyridyl]sulfonyl]-2,6- diazaspiro[3.3]heptan-6- yl]methanone | 538.3 [M + H]⁺ |
| 261 | 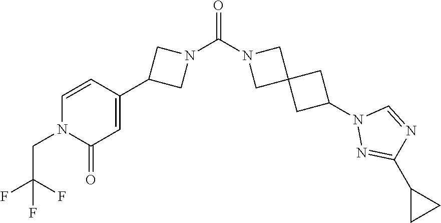 [SMILES: O=C(N1CC(c2ccn(CC(F)(F)F)c(=O)c2)C1)N1CC2(CC(n3cnc(C4CC4)n3)C2)C1] | 4-[1-[6-(3-cyclopropyl- 1,2,4-triazol-1-yl)-2- azaspiro[3.3]heptane-2- carbonyl]azetidin-3-yl]-1- (2,2,2- trifluoroethyl)pyridin-2-one | 463.3 [M + H]⁺ |
| 263 | 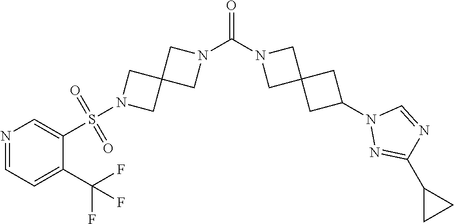 [SMILES: O=C(N1CC2(CC(n3cnc(C4CC4)n3)C2)C1)N1CC2(C1)CN(S(=O)(=O)c1cnccc1C(F)(F)F)C2] | [6-(3-cyclopropyl-1,2,4- triazol-1-yl)-2- azaspiro[3.3]heptan-2-yl]- [2-[[4-(trifluoromethyl)-3- pyridyl]sulfonyl]-2,6- diazaspiro[3.3]heptan-6- yl]methanone | 538.4 [M + H]⁺ |
| 264 | 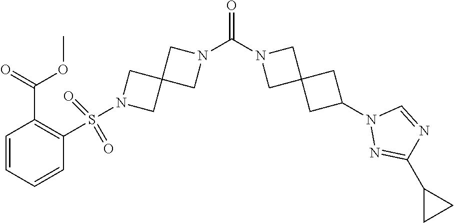 [SMILES: COC(=O)c1ccccc1S(=O)(=O)N1CC2(CN(C(=O)N3CC4(CC(n5cnc(C6CC6)n5)C4)C3)C2)C1] | methyl 2-[[6-[6-(3- cyclopropyl-1,2,4-triazol-1- yl)-2-azaspiro[3.3]heptane- 2-carbonyl]-2,6- diazaspiro[3.3]heptan-2- yl]sulfonyl]benzoate | 527.4 [M + H]⁺ |
| 265 | 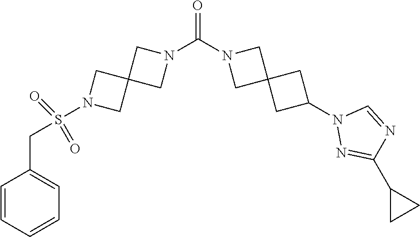 [SMILES: O=C(N1CC2(CC(n3cnc(C4CC4)n3)C2)C1)N1CC2(C1)CN(S(=O)(=O)Cc1ccccc1)C2] | (2-benzylsulfonyl-2,6- diazaspiro[3.3]heptan-6-yl)- [6-(3-cyclopropyl-1,2,4- triazol-1-yl)-2- azaspiro[3.3]heptan-2- yl]methanone | 483.3 [M + H]⁺ |
| 272 | 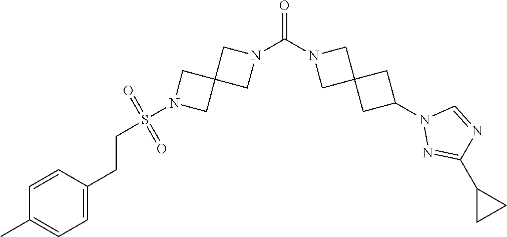 | [6-(3-cyclopropyl-1,2,4- triazol-1-yl)-2- azaspiro[3.3]heptan-2-yl]- [2-[2-(4- fluorophenyl)ethylsulfonyl]- 2,6-diazaspiro[3.3]heptan-6- yl]methanone | 515.4 [M + H]⁺ |
| 274 | 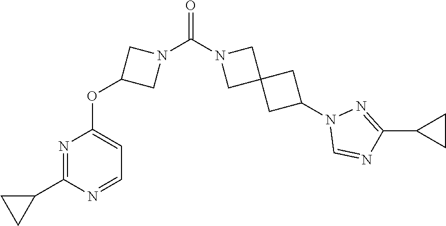 [SMILES: O=C(N1CC(Oc2ccnc(C3CC3)n2)C1)N1CC2(CC(n3cnc(C4CC4)n3)C2)C1] | [3-(2-cyclopropylpyrimidin- 4-yl)oxyazetidin-1-yl]-[6- (3-cyclopropyl-1,2,4-triazol- 1-yl)-2-azaspiro[3.3]heptan- 2-yl]methanone | 422.2 [M + H]⁺ |
| 275 | 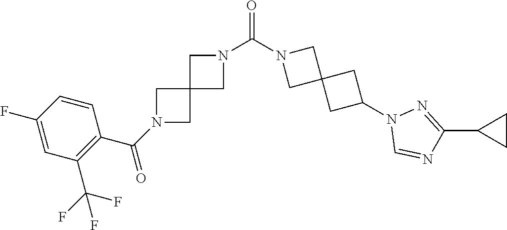 [SMILES: O=C(c1ccc(F)cc1C(F)(F)F)N1CC2(C1)CN(C(=O)N1CC3(CC(n4cnc(C5CC5)n4)C3)C1)C2] | [2-[6-(3-cyclopropyl-1,2,4- triazol-1-yl)-2- azaspiro[3.3]heptane-2- carbonyl]-2,6- diazaspiro[3.3]heptan-6-yl]- [4-fluoro-2- (trifluoromethyl)phenyl] methanone | 519.3 [M + H]⁺ |
| 276 | 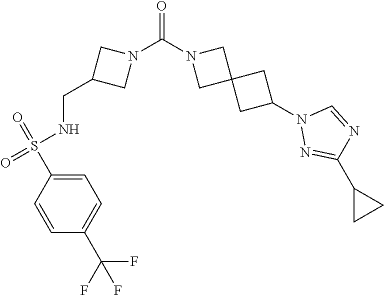 [SMILES: O=C(N1CC(CNS(=O)(=O)c2ccc(C(F)(F)F)cc2)C1)N1CC2(CC(n3cnc(C4CC4)n3)C2)C1] | N-[[1-[6-(3-cyclopropyl- 1,2,4-triazol-1-yl)-2- azaspiro[3.3]heptane-2- carbonyl]azetidin-3- yl]methyl]-4- (trifluoromethyl)benzene- sulfonamide | 525.4 [M + H]⁺ |
| 278 | 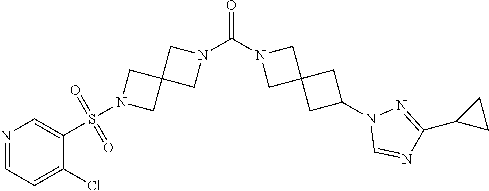 [SMILES: O=C(N1CC2(CC(n3cnc(C4CC4)n3)C2)C1)N1CC2(C1)CN(S(=O)(=O)c1cnccc1Cl)C2] | [2-[(4-chloro-3- pyridyl)sulfonyl]-2,6- diazaspiro[3.3]heptan-6-yl]- [6-(3-cyclopropyl-1,2,4- triazol-1-yl)-2- azaspiro[3.3]heptan-2- yl]methanone | 504.4 [M + H]⁺ |
| 279 | 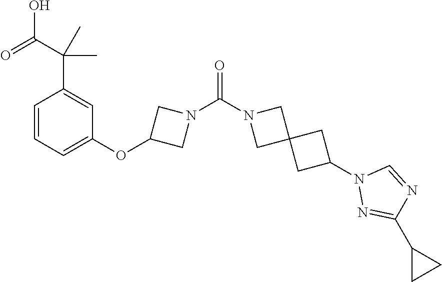 [SMILES: CC(C)(C(=O)O)c1cccc(OC2CN(C(=O)N3CC4(CC(n5cnc(C6CC6)n5)C4)C3)C2)c1] | 2-[3-[1-[6-(3-cyclopropyl- 1,2,4-triazol-1-yl)-2- azaspiro[3.3]heptane-2- carbonyl]azetidin-3- yl]oxyphenyl]-2-methyl- propanoic acid | 466.3 [M + H]⁺ |
| 280 | 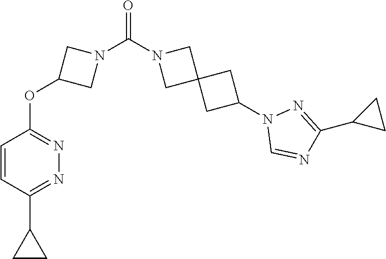 [SMILES: O=C(N1CC(Oc2ccc(C3CC3)nn2)C1)N1CC2(CC(n3cnc(C4CC4)n3)C2)C1] | [3-(6-cyclopropylpyridazin- 3-yl)oxyazetidin-1-yl]-[6- (3-cyclopropyl-1,2,4-triazol- 1-yl)-2-azaspiro[3.3]heptan- 2-yl]methanone | 422.4 [M + H]⁺ |
| 281 | 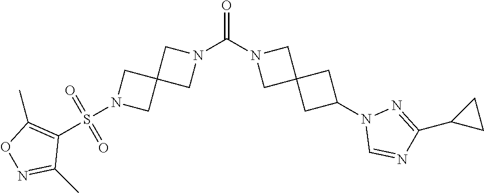 [SMILES: Cc1noc(C)c1S(=O)(=O)N1CC2(CN(C(=O)N3CC4(CC(n5cnc(C6CC6)n5)C4)C3)C2)C1] | [6-(3-cyclopropyl-1,2,4- triazol-1-yl)-2- azaspiro[3.3]heptan-2-yl]- [2-(3,5-dimethylisoxazol-4- yl)sulfonyl-2,6- diazaspiro[3.3]heptan-6- yl]methanone | 488.4 [M + H]⁺ |
| 282 | 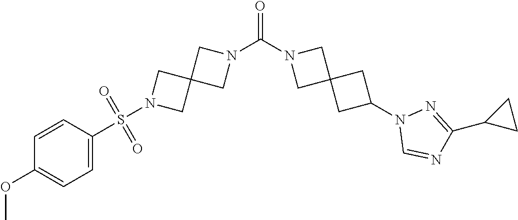 [SMILES: COc1ccc(S(=O)(=O)N2CC3(CN(C(=O)N4CC5(CC(n6cnc(C7CC7)n6)C5)C4)C3)C2)cc1] | [6-(3-cyclopropyl-1,2,4- triazol-1-yl)-2- azaspiro[3.3]heptan-2-yl]- [2-(4- methoxyphenyl)sulfonyl- 2,6-diazaspiro[3.3]heptan-6- yl]methanone | 499.3 [M + H]⁺ |
| 283 | 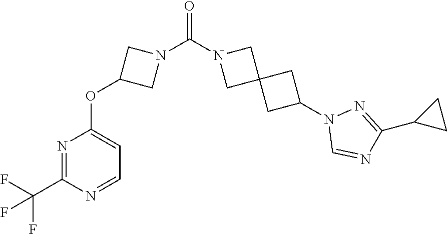 [SMILES: O=C(N1CC(Oc2ccnc(C(F)(F)F)n2)C1)N1CC2(CC(n3cnc(C4CC4)n3)C2)C1] | [6-(3-cyclopropyl-1,2,4- triazol-1-yl)-2- azaspiro[3.3]heptan-2-yl]- [3-[2- (trifluoromethyl)pyrimidin- 4-yl]oxyazetidin-1- yl]methanone | 450.2 [M + H]⁺ |
| 284 | 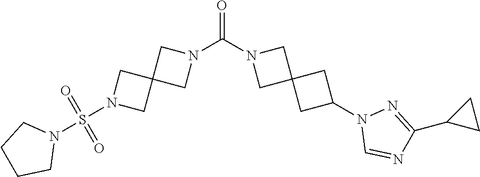 [SMILES: O=C(N1CC2(CC(n3cnc(C4CC4)n3)C2)C1)N1CC2(C1)CN(S(=O)(=O)N1CCCC1)C2] | [6-(3-cyclopropyl-1,2,4- triazol-1-yl)-2- azaspiro[3.3]heptan-2-yl]- (2-pyrrolidin-1-ylsulfonyl- 2,6-diazaspiro[3.3]heptan-6- yl)methanone | 462.4 [M + H]⁺ |
| 285 | 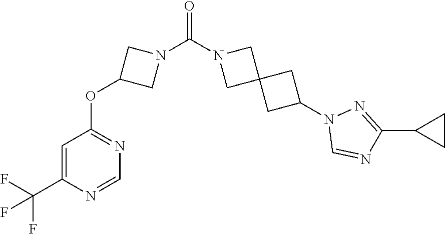 [SMILES: O=C(N1CC(Oc2cc(C(F)(F)F)ncn2)C1)N1CC2(CC(n3cnc(C4CC4)n3)C2)C1] | [6-(3-cyclopropyl-1,2,4- triazol-1-yl)-2- azaspiro[3.3]heptan-2-yl]- [3-[6- (trifluoromethyl)pyrimidin- 4-yl]oxyazetidin-1- yl]methanone | 450.2 [M + H]⁺ |
| 286 | 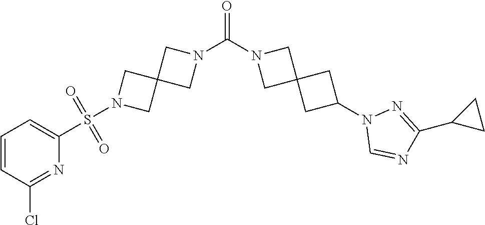 [SMILES: O=C(N1CC2(CC(n3cnc(C4CC4)n3)C2)C1)N1CC2(C1)CN(S(=O)(=O)c1cccc(Cl)n1)C2] | [2-[(6-chloro-2- pyridyl)sulfonyl]-2,6- diazaspiro[3.3]heptan-6-yl]- [6-(3-cyclopropyl-1,2,4- triazol-1-yl)-2- azaspiro[3.3]heptan-2- yl]methanone | 504.3 [M + H]⁺ |
| 287 | 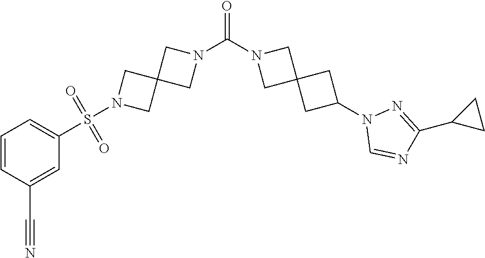 [SMILES: N#Cc1cccc(S(=O)(=O)N2CC3(CN(C(=O)N4CC5(CC(n6cnc(C7CC7)n6)C5)C4)C3)C2)c1] | 3-[[6-[6-(3-cyclopropyl- 1,2,4-triazol-1-yl)-2- azaspiro[3.3]heptane-2- carbonyl]-2,6- diazaspiro[3.3]heptan-2- yl]sulfonyl]benzonitrile | 494.4 [M + H]⁺ |
| 288 | 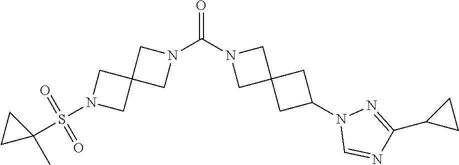 [SMILES: CC1(S(=O)(=O)N2CC3(CN(C(=O)N4CC5(CC(n6cnc(C7CC7)n6)C5)C4)C3)C2)CC1] | [6-(3-cyclopropyl-1,2,4- triazol-1-yl)-2- azaspiro[3.3]heptan-2-yl]- [2-(1- methylcyclopropyl)sulfonyl- 2,6-diazaspiro[3.3]heptan-6- yl]methanone | 447.3 [M + H]⁺ |
| 289 | 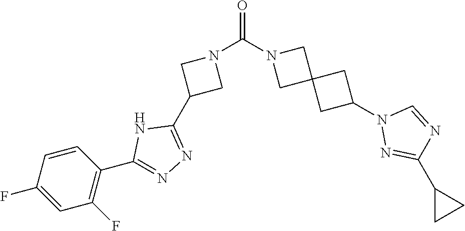 [SMILES: O=C(N1CC(c2nnc(-c3ccc(F)cc3F)[nH]2)C1)N1CC2(CC(n3cnc(C4CC4)n3)C2)C1] | [6-(3-cyclopropyl-1,2,4- triazol-1-yl)-2- azaspiro[3.3]heptan-2-yl]- [3-[5-(2,4-difluorophenyl)- 4H-1,2,4-triazol-3- yl]azetidin-1-yl]methanone | 467.1 [M + H]⁺ |
| 290 | 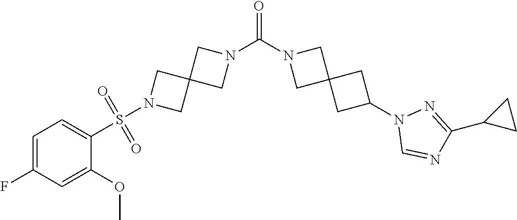 [SMILES: COc1cc(F)ccc1S(=O)(=O)N1CC2(CN(C(=O)N3CC4(CC(n5cnc(C6CC6)n5)C4)C3)C2)C1] | [6-(3-cyclopropyl-1,2,4- triazol-1-yl)-2- azaspiro[3.3]heptan-2-yl]- [2-(4-fluoro-2-methoxy- phenyl)sulfonyl-2,6- diazaspiro[3.3]heptan-6- yl]methanone | 517.3 [M + H]⁺ |
| 292 | 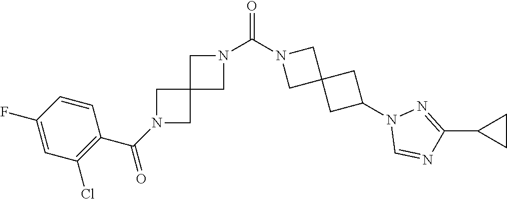 [SMILES: O=C(c1ccc(F)cc1Cl)N1CC2(C1)CN(C(=O)N1CC3(CC(n4cnc(C5CC5)n4)C3)C1)C2] | (2-chloro-4-fluoro-phenyl)- [2-[6-(3-cyclopropyl-1,2,4- triazol-1-yl)-2- azaspiro[3.3]heptane-2- carbonyl]-2,6- diazaspiro[3.3]heptan-6- yl]methanone | 485.2 [M + H]⁺ |
| 293 | 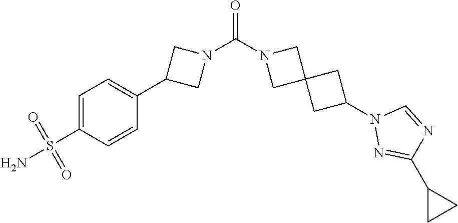 [SMILES: NS(=O)(=O)c1ccc(C2CN(C(=O)N3CC4(CC(n5cnc(C6CC6)n5)C4)C3)C2)cc1] | 4-[1-[6-(3-cyclopropyl- 1,2,4-triazol-1-yl)-2- azaspiro[3.3]heptane-2- carbonyl]azetidin-3- yl]benzenesulfonamide | 443.3 [M + H]⁺ |
| 294 | 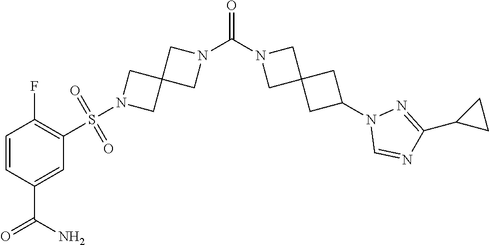 [SMILES: NC(=O)c1ccc(F)c(S(=O)(=O)N2CC3(CN(C(=O)N4CC5(CC(n6cnc(C7CC7)n6)C5)C4)C3)C2)c1] | 3-[6-[6-(3-cyclopropyl- 1,2,4-triazol-1-yl)-2- azaspiro[3.3]heptane-2- carbonyl]-2,6- diazaspiro[3.3]heptan-2- yl]sulfonyl]-4-fluoro- benzamide | 530.3 [M + H]⁺ |
| 295 | 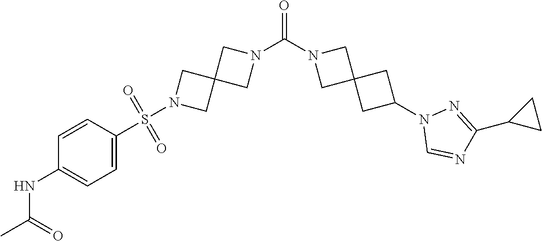 [SMILES: CC(=O)Nc1ccc(S(=O)(=O)N2CC3(CN(C(=O)N4CC5(CC(n6cnc(C7CC7)n6)C5)C4)C3)C2)cc1] | N-[4-[[6-[6-(3-cyclopropyl- 1,2,4-triazol-1-yl)-2- azaspiro[3.3]heptane-2- carbonyl]-2,6- diazaspiro[3.3]heptan-2- yl]sulfonyl]phenyl]acetamide | 526.4 [M + H]⁺ |
| 296 | 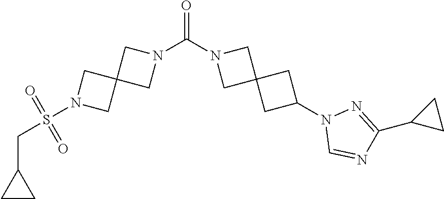 [SMILES: O=C(N1CC2(CC(n3cnc(C4CC4)n3)C2)C1)N1CC2(C1)CN(S(=O)(=O)CC1CC1)C2] | [2- (cyclopropylmethylsulfonyl)- 2,6-diazaspiro[3.3]heptan- 6-yl]-[6-(3-cyclopropyl- 1,2,4-triazol-1-yl)-2- azaspiro[3.3]heptan-2- yl]methanone | 447.3 [M + H]⁺ |
| 297 | 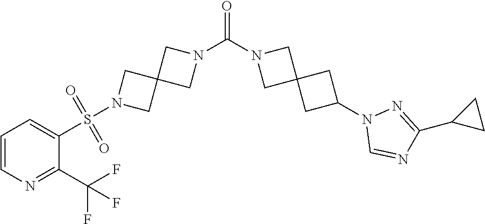 [SMILES: O=C(N1CC2(CC(n3cnc(C4CC4)n3)C2)C1)N1CC2(C1)CN(S(=O)(=O)c1cccnc1C(F)(F)F)C2] | [6-(3-cyclopropyl-1,2,4- triazol-1-yl)-2- azaspiro[3.3]heptan-2-yl]- [2-[[2-(trifluoromethyl)-3- pyridyl]sulfonyl]-2,6- diazaspiro[3.3]heptan-6- yl]methanone | 538.3 [M + H]⁺ |
| 298 | 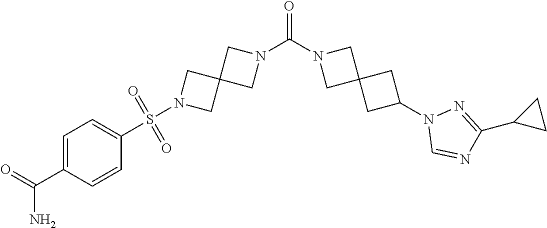 [SMILES: NC(=O)c1ccc(S(=O)(=O)N2CC3(CN(C(=O)N4CC5(CC(n6cnc(C7CC7)n6)C5)C4)C3)C2)cc1] | 4-[[6-[6-(3-cyclopropyl- 1,2,4-triazol-1-yl)-2- azaspiro[3.3]heptane-2- carbonyl]-2,6- diazaspiro[3.3]heptan-2- yl]sulfonyl]benzamide | 512.3 [M + H]⁺ |
| 299 | 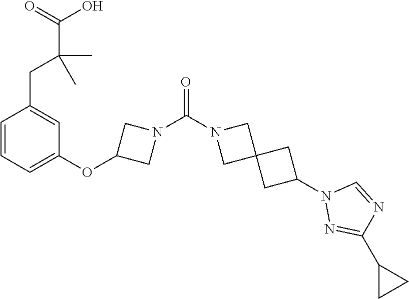 [SMILES: CC(C)(Cc1cccc(OC2CN(C(=O)N3CC4(CC(n5cnc(C6CC6)n5)C4)C3)C2)c1)C(=O)O] | 3-[3-[1-[6-(3-cyclopropyl- 1,2,4-triazol-1-yl)-2- azaspiro[3.3]heptane-2- carbonyl]azetidin-3- yl]oxyphenyl]-2,2-dimethyl- propanoic acid | 480.4 [M + H]⁺ |
| 300 | 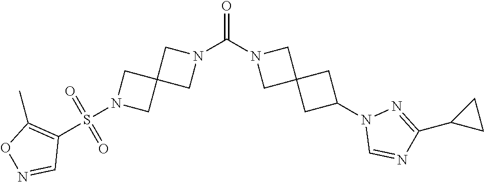 [SMILES: Cc1oncc1S(=O)(=O)N1CC2(CN(C(=O)N3CC4(CC(n5cnc(C6CC6)n5)C4)C3)C2)C1] | [6-(3-cyclopropyl-1,2,4- triazol-1-yl)-2- azaspiro[3.3]heptan-2-yl]- [2-(5-methylisoxazol-4- yl)sulfonyl-2,6- diazaspiro[3.3]heptan-6- yl]methanone | 474.3 [M + H]⁺ |
| 302 | 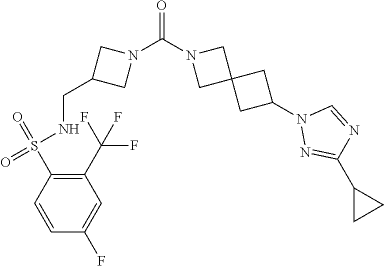 [SMILES: O=C(N1CC(CNS(=O)(=O)c2ccc(F)cc2C(F)(F)F)C1)N1CC2(CC(n3cnc(C4CC4)n3)C2)C1] | N-[1-[6-(3-cyclopropyl- 1,2,4-triazol-1-yl)-2- azaspiro[3.3]heptane-2- carbonyl]azetidin-3- yl]methyl]-4-fluoro-2- (trifluoromethyl)benzene- sulfonamide | 543.4 [M + H]⁺ |
| 303 | 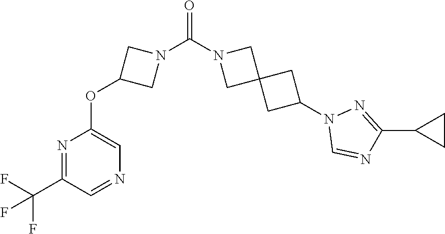 [SMILES: O=C(N1CC(Oc2cncc(C(F)(F)F)n2)C1)N1CC2(CC(n3cnc(C4CC4)n3)C2)C1] | [6-(3-cyclopropyl-1,2,4- triazol-1-yl)-2- azaspiro[3.3]heptan-2-yl]- [3-[6- (trifluoromethyl)pyrazin-2- yl]oxyazetidin-1- yl]methanone | 450.2 [M + H]⁺ |
| 304 | 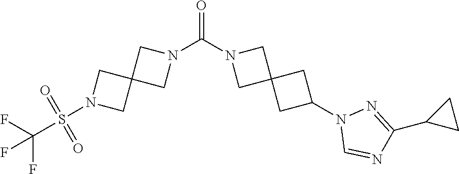 [SMILES: O=C(N1CC2(CC(n3cnc(C4CC4)n3)C2)C1)N1CC2(C1)CN(S(=O)(=O)C(F)(F)F)C2] | [6-(3-cyclopropyl-1,2,4- triazol-1-yl)-2- azaspiro[3.3]heptan-2-yl]- [2- (trifluoromethylsulfonyl)- 2,6-diazaspiro[3.3]heptan-6- yl]methanone | 461.3 [M + H]⁺ |
| 306 | 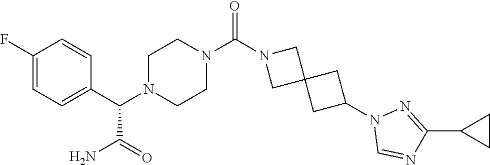 [SMILES: NC(=O)[C@H](c1ccc(F)cc1)N1CCN(C(=O)N2CC3(CC(n4cnc(C5CC5)n4)C3)C2)CC1] | (2S)-2-[4-[6-(3-cyclopropyl- 1,2,4-triazol-1-yl)-2- azaspiro[3.3]heptane-2- carbonyl]piperazin-1-yl]-2- (4-fluorophenyl)acetamide | 468.5 [M + H]⁺ |
| 307 | 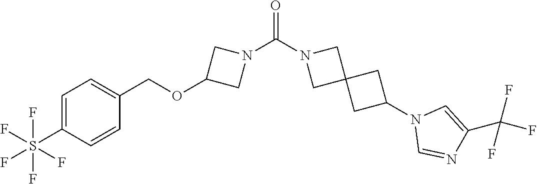 [SMILES: O=C(N1CC(OCc2ccc(S(F)(F)(F)(F)F)cc2)C1)N1CC2(CC(n3cnc(C(F)(F)F)c3)C2)C1] | [3-[[4-(pentafluoro-λ⁶- sulfanyl)phenyl]methoxy] azetidin-1-yl]-[6-[4- (trifluoromethyl)imidazol-1- yl]-2-azaspiro[3.3]heptan-2- yl]methanone | 547.3 [M + H]⁺ |
| 308 | 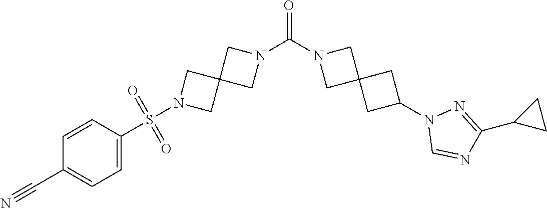 [SMILES: N#Cc1ccc(S(=O)(=O)N2CC3(CN(C(=O)N4CC5(CC(n6cnc(C7CC7)n6)C5)C4)C3)C2)cc1] | 4-[6-[6-(3-cyclopropyl- 1,2,4-triazol-1-yl)-2- azaspiro[3.3]heptane-2- carbonyl]-2,6- diazaspiro[3.3]heptan-2- yl]sulfonyl]benzonitrile | 494.4 [M + H]⁺ |
| 309 | 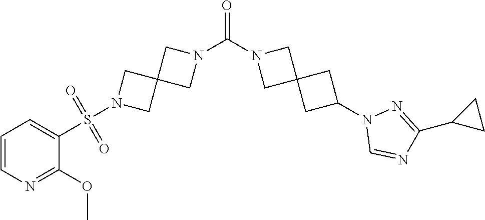 [SMILES: COc1ncccc1S(=O)(=O)N1CC2(CN(C(=O)N3CC4(CC(n5cnc(C6CC6)n5)C4)C3)C2)C1] | [6-(3-cyclopropyl-1,2,4- triazol-1-yl)-2- azaspiro[3.3]heptan-2-yl]- [2-[(2-methoxy-3- pyridyl)sulfonyl]-2,6- diazaspiro[3.3]heptan-6- yl]methanone | 500.4 [M + H]⁺ |
| 310 | 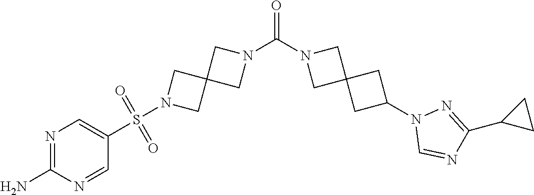 [SMILES: Nc1ncc(S(=O)(=O)N2CC3(CN(C(=O)N4CC5(CC(n6cnc(C7CC7)n6)C5)C4)C3)C2)cn1] | [2-(2-aminopyrimidin-5- yl)sulfonyl-2,6- diazaspiro[3.3]heptan-6-yl]- [6-(3-cyclopropyl-1,2,4- triazol-1-yl)-2- azaspiro[3.3]heptan-2- yl]methanone | 486.4 [M + H]⁺ |
| 311 | 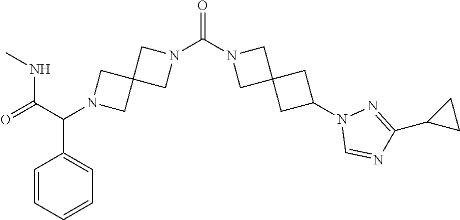 [SMILES: CNC(=O)C(c1ccccc1)N1CC2(CN(C(=O)N3CC4(CC(n5cnc(C6CC6)n5)C4)C3)C2)C1] | 2-[2-[6-(3-cyclopropyl- 1,2,4-triazol-1-yl)-2- azaspiro[3.3]heptane-2- carbonyl]-2,6- diazaspiro[3.3]heptan-6-yl]- N-methyl-2-phenyl- acetamide | 476.4 [M + H]⁺ |
| 312 | 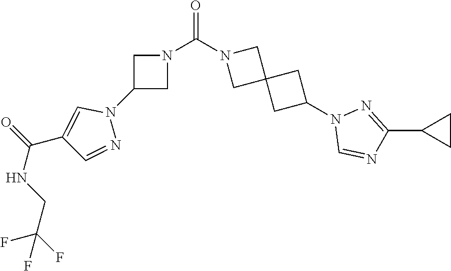 [SMILES: O=C(NCC(F)(F)F)c1cnn(C2CN(C(=O)N3CC4(CC(n5cnc(C6CC6)n5)C4)C3)C2)c1] | 1-[1-[6-(3-cyclopropyl- 1,2,4-triazol-1-yl)-2- azaspiro[3.3]heptane-2- carbonyl]azetidin-3-yl]-N- (2,2,2- trifluoroethyl)pyrazole-4- carboxamide | 479.3 [M + H]⁺ |
| 313 | 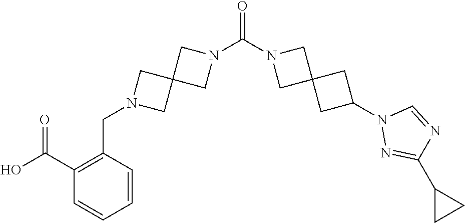 [SMILES: O=C(O)c1ccccc1CN1CC2(C1)CN(C(=O)N1CC3(CC(n4cnc(C5CC5)n4)C3)C1)C2] | 2-[[2-[6-(3-cyclopropyl- 1,2,4-triazol-1-yl)-2- azaspiro[3.3]heptane-2- carbonyl]-2,6- diazaspiro[3.3]heptan-6- yl]methyl]benzoic acid | 461.3 [M + H]⁺ |
| 314 | 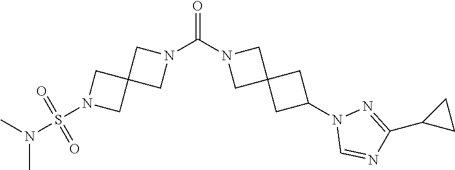 [SMILES: CN(C)S(=O)(=O)N1CC2(CN(C(=O)N3CC4(CC(n5cnc(C6CC6)n5)C4)C3)C2)C1] | 6-[6-(3-cyclopropyl-1,2,4- triazol-1-yl)-2- azaspiro[3.3]heptane-2- carbonyl]-N,N-dimethyl- 2,6-diazaspiro[3.3]heptane- 2-sulfonamide | 436.4 [M + H]⁺ |
| 315 | 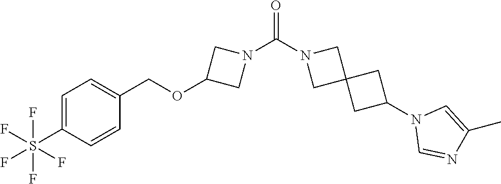 [SMILES: Cc1cn(C2CC3(C2)CN(C(=O)N2CC(OCc4ccc(S(F)(F)(F)(F)F)cc4)C2)C3)cn1] | [6-(4-methylimidazol-1-yl)- 2-azaspiro[3.3]heptan-2-yl]- [3-[[4-(pentafluoro-26- sulfanyl)phenyl]methoxy] azetidin-1-yl]methanone | 493.2 [M + H]⁺ |
| 316 | 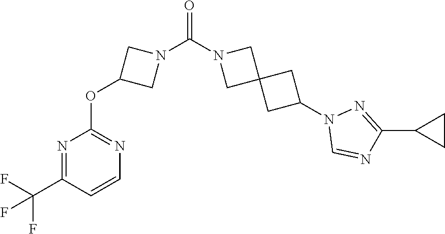 [SMILES: O=C(N1CC(Oc2nccc(C(F)(F)F)n2)C1)N1CC2(CC(n3cnc(C4CC4)n3)C2)C1] | [6-(3-cyclopropyl-1,2,4- triazol-1-yl)-2- azaspiro[3.3]heptan-2-yl]- [3-[4- (trifluoromethyl)pyrimidin- 2-yl]oxyazetidin-1- yl]methanone | 450.2 [M + H]⁺ |
| 317 | 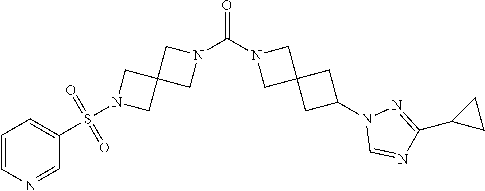 [SMILES: O=C(N1CC2(CC(n3cnc(C4CC4)n3)C2)C1)N1CC2(C1)CN(S(=O)(=O)c1cccnc1)C2] | [6-(3-cyclopropyl-1,2,4- triazol-1-yl)-2- azaspiro[3.3]heptan-2-yl]- [2-(3-pyridylsulfonyl)-2,6- diazaspiro[3.3]heptan-6- yl]methanone | 470.4 [M + H]⁺ |
| 318 | 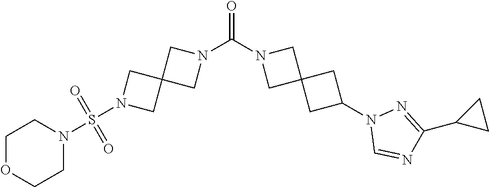 [SMILES: O=C(N1CC2(CC(n3cnc(C4CC4)n3)C2)C1)N1CC2(C1)CN(S(=O)(=O)N1CCOCC1)C2] | [6-(3-cyclopropyl-1,2,4- triazol-1-yl)-2- azaspiro[3.3]heptan-2-yl]- (2-morpholinosulfonyl-2,6- diazaspiro[3.3]heptan-6- yl)methanone | 478.4 [M + H]⁺ |
| 319 | 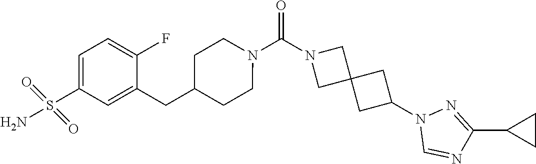 [SMILES: NS(=O)(=O)c1ccc(F)c(CC2CCN(C(=O)N3CC4(CC(n5cnc(C6CC6)n5)C4)C3)CC2)c1] | 3-[1-[6-(3-cyclopropyl- 1,2,4-triazol-1-yl)-2- azaspiro[3.3]heptane-2- carbonyl]-4- piperidyl]methyl]-4-fluoro- benzenesulfonamide | 503.4 [M + H]⁺ |
| 320 | 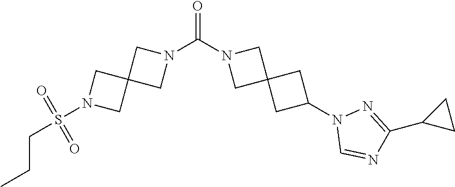 [SMILES: CCCS(=O)(=O)N1CC2(CN(C(=O)N3CC4(CC(n5cnc(C6CC6)n5)C4)C3)C2)C1] | [6-(3-cyclopropyl-1,2,4- triazol-1-yl)-2- azaspiro[3.3]heptan-2-yl]- (2-propylsulfonyl-2,6- diazaspiro[3.3]heptan-6- yl)methanone | 435.4 [M + H]⁺ |
| 322 | 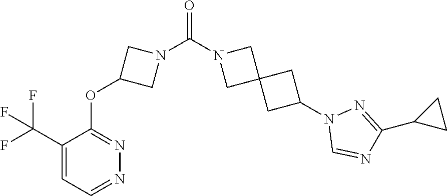 [SMILES: O=C(N1CC(Oc2nnccc2C(F)(F)F)C1)N1CC2(CC(n3cnc(C4CC4)n3)C2)C1] | [6-(3-cyclopropyl-1,2,4- triazol-1-yl)-2- azaspiro[3.3]heptan-2-yl]- [3-[4- (trifluoromethyl)pyridazin- 3-yl]oxyazetidin-1- yl]methanone | 450.3 [M + H]⁺ |
| 323 | 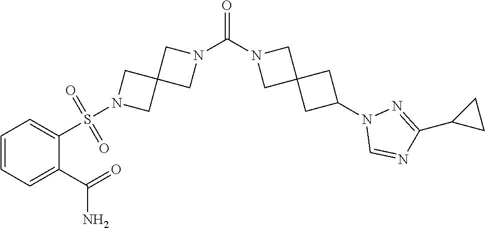 [SMILES: NC(=O)c1ccccc1S(=O)(=O)N1CC2(CN(C(=O)N3CC4(CC(n5cnc(C6CC6)n5)C4)C3)C2)C1] | 2-[[6-[6-(3-cyclopropyl- 1,2,4-triazol-1-yl)-2- azaspiro[3.3]heptane-2- carbonyl]-2,6- diazaspiro[3.3]heptan-2- yl]sulfonyl]benzamide | 512.4 [M + H]⁺ |
| 324 | 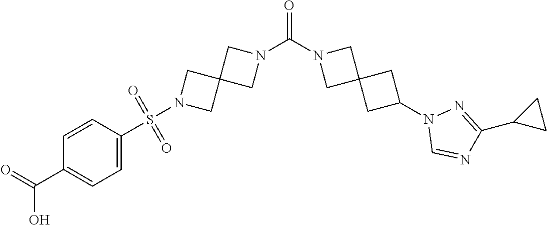 [SMILES: O=C(O)c1ccc(S(=O)(=O)N2CC3(CN(C(=O)N4CC5(CC(n6cnc(C7CC7)n6)C5)C4)C3)C2)cc1] | 4-[6-[6-(3-cyclopropyl- 1,2,4-triazol-1-yl)-2- azaspiro[3.3]heptane-2- carbonyl]-2,6- diazaspiro[3.3]heptan-2- yl]sulfonyl]benzoic acid | 513.4 [M + H]⁺ |
| 325 | 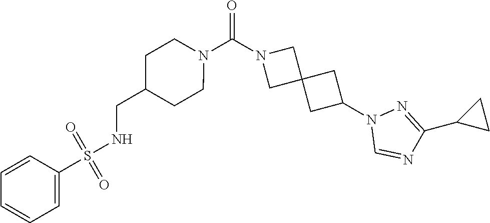 [SMILES: O=C(N1CCC(CNS(=O)(=O)c2ccccc2)CC1)N1CC2(CC(n3cnc(C4CC4)n3)C2)C1] | N-[[1-[6-(3-cyclopropyl- 1,2,4-triazol-1-yl)-2- azaspiro[3.3]heptane-2- carbonyl]-4- piperidyl]methyl]benzene- sulfonamide | 485.4 [M + H]⁺ |
| 326 | 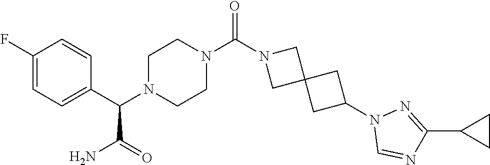 [SMILES: NC(=O)[C@@H](c1ccc(F)cc1)N1CCN(C(=O)N2CC3(CC(n4cnc(C5CC5)n4)C3)C2)CC1] | (2R)-2-[4-[6-(3- cyclopropyl-1,2,4-triazol-1- yl)-2-azaspiro[3.3]heptane- 2-carbonyl]piperazin-1-yl]- 2-(4- fluorophenyl)acetamide | 468.5 [M + H]⁺ |
| 327 | 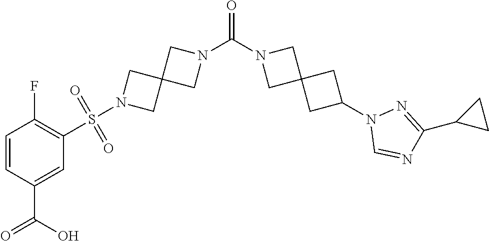 [SMILES: O=C(O)c1ccc(F)c(S(=O)(=O)N2CC3(CN(C(=O)N4CC5(CC(n6cnc(C7CC7)n6)C5)C4)C3)C2)c1] | 3-[6-[6-(3-cyclopropyl- 1,2,4-triazol-1-yl)-2- azaspiro[3.3]heptane-2- carbonyl]-2,6- diazaspiro[3.3]heptan-2- yl]sulfonyl]-4-fluoro- benzoic acid | 531.3 [M + H]⁺ |
| 328 | 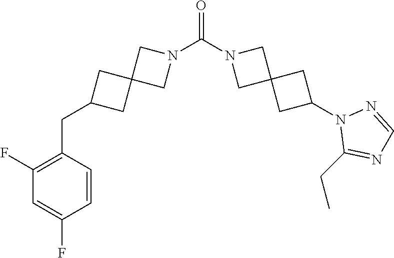 [SMILES: CCc1ncnn1C1CC2(C1)CN(C(=O)N1CC3(CC(Cc4ccc(F)cc4F)C3)C1)C2] | [6-[(2,4- difluorophenyl)methyl]-2- azaspiro[3.3]heptan-2-yl]- [6-(5-ethyl-1,2,4-triazol-1- yl)-2-azaspiro[3.3]heptan-2- yl]methanone | 442.3 [M + H]⁺ |
| 329 | 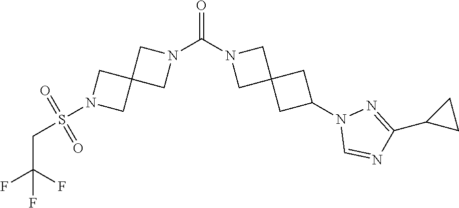 [SMILES: O=C(N1CC2(CC(n3cnc(C4CC4)n3)C2)C1)N1CC2(C1)CN(S(=O)(=O)CC(F)(F)F)C2] | [6-(3-cyclopropyl-1,2,4- triazol-1-yl)-2- azaspiro[3.3]heptan-2-yl]- [2-(2,2,2- trifluoroethylsulfonyl)-2,6- diazaspiro[3.3]heptan-6- yl]methanone | 475.4 [M + H]⁺ |
| 330 | 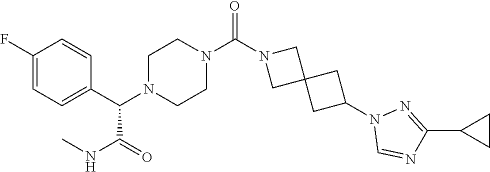 [SMILES: CNC(=O)[C@H](c1ccc(F)cc1)N1CCN(C(=O)N2CC3(CC(n4cnc(C5CC5)n4)C3)C2)CC1] | (2S)-2-[4-[6-(3-cyclopropyl- 1,2,4-triazol-1-yl)-2- azaspiro[3.3]heptane-2- carbonyl]piperazin-1-yl]-2- (4-fluorophenyl)-N-methyl- acetamide | 482.5 [M + H]⁺ |
| 331 | 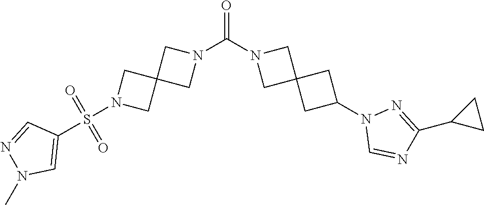 [SMILES: Cn1cc(S(=O)(=O)N2CC3(CN(C(=O)N4CC5(CC(n6cnc(C7CC7)n6)C5)C4)C3)C2)cn1] | [6-(3-cyclopropyl-1,2,4- triazol-1-yl)-2- azaspiro[3.3]heptan-2-yl]- [2-(1-methylpyrazol-4- yl)sulfonyl-2,6- diazaspiro[3.3]heptan-6- yl]methanone | 473.4 [M + H]⁺ |
| 332 | 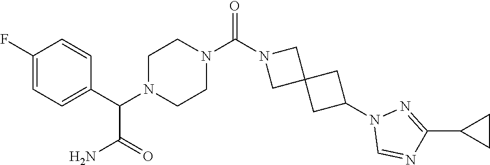 [SMILES: NC(=O)C(c1ccc(F)cc1)N1CCN(C(=O)N2CC3(CC(n4cnc(C5CC5)n4)C3)C2)CC1] | 2-[4-[6-(3-cyclopropyl- 1,2,4-triazol-1-yl)-2- azaspiro[3.3]heptane-2- carbonyl]piperazin-1-yl]-2- (4-fluorophenyl)acetamide | 468.4 [M + H]⁺ |
| 333 | 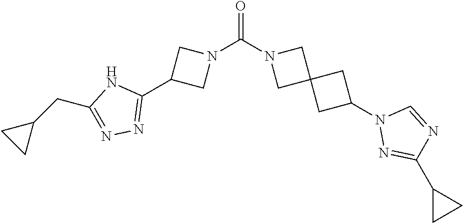 [SMILES: O=C(N1CC(c2nnc(CC3CC3)[nH]2)C1)N1CC2(CC(n3cnc(C4CC4)n3)C2)C1] | [3-[5-(cyclopropylmethyl)- 4H-1,2,4-triazol-3- yl]azetidin-1-yl]-[6-(3- cyclopropyl-1,2,4-triazol-1- yl)-2-azaspiro[3.3]heptan-2- yl]methanone | 409.4 [M + H]⁺ |
| 334 | 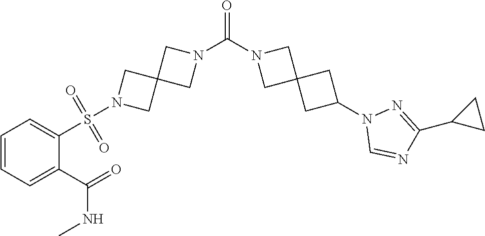 [SMILES: CNC(=O)c1ccccc1S(=O)(=O)N1CC2(CN(C(=O)N3CC4(CC(n5cnc(C6CC6)n5)C4)C3)C2)C1] | 2-[6-[6-(3-cyclopropyl- 1,2,4-triazol-1-yl)-2- azaspiro[3.3]heptane-2- carbonyl]-2,6- diazaspiro[3.3]heptan-2- yl]sulfonyl]-N-methyl- benzamide | 526.4 [M + H]⁺ |
| 335 | 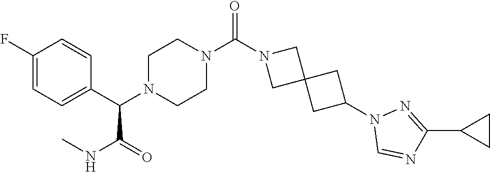 [SMILES: CNC(=O)[C@@H](c1ccc(F)cc1)N1CCN(C(=O)N2CC3(CC(n4cnc(C5CC5)n4)C3)C2)CC1] | (2R)-2-[4-[6-(3- cyclopropyl-1,2,4-triazol-1- yl)-2-azaspiro[3.3]heptane- 2-carbonyl]piperazin-1-yl]- 2-(4-fluorophenyl)-N- methyl-acetamide | 482.4 [M + H]⁺ |
| 336 | 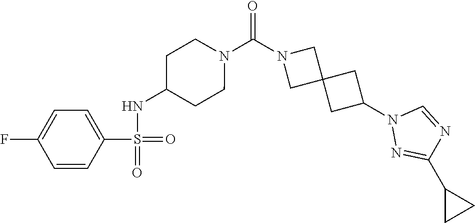 [SMILES: O=C(N1CCC(NS(=O)(=O)c2ccc(F)cc2)CC1)N1CC2(CC(n3cnc(C4CC4)n3)C2)C1] | N-[1-[6-(3-cyclopropyl- 1,2,4-triazol-1-yl)-2- azaspiro[3.3]heptane-2- carbonyl]-4-piperidyl]-4- fluoro-benzenesulfonamide | 489.3 [M + H]⁺ |
| 337 | 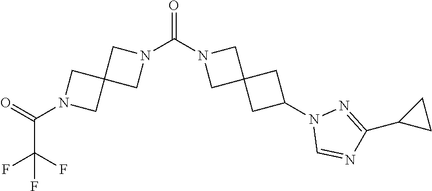 [SMILES: O=C(N1CC2(CC(n3cnc(C4CC4)n3)C2)C1)N1CC2(C1)CN(C(=O)C(F)(F)F)C2] | 1-[2-[6-(3-cyclopropyl- 1,2,4-triazol-1-yl)-2- azaspiro[3.3]heptane-2- carbonyl]-2,6- diazaspiro[3.3]heptan-6-yl]- 2,2,2-trifluoro-ethanone | 425.3 [M + H]⁺ |
| 338 | 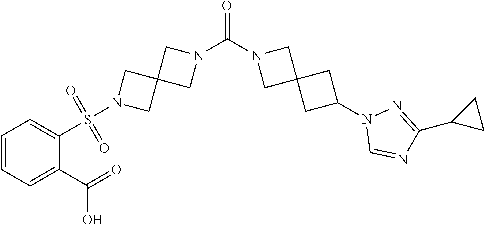 [SMILES: O=C(O)c1ccccc1S(=O)(=O)N1CC2(CN(C(=O)N3CC4(CC(n5cnc(C6CC6)n5)C4)C3)C2)C1] | 2-[[6-[6-(3-cyclopropyl- 1,2,4-triazol-1-yl)-2- azaspiro[3.3]heptane-2- carbonyl]-2,6- diazaspiro[3.3]heptan-2- yl]sulfonyl]benzoic acid | 513.4 [M + H]⁺ |
| 339 | 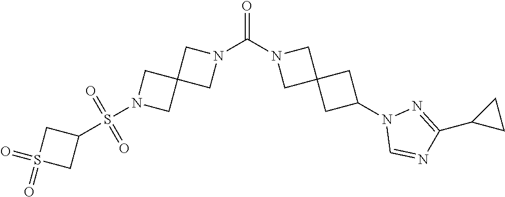 [SMILES: O=C(N1CC2(CC(n3cnc(C4CC4)n3)C2)C1)N1CC2(C1)CN(S(=O)(=O)C1CS(=O)(=O)C1)C2] | [6-(3-cyclopropyl-1,2,4- triazol-1-yl)-2- azaspiro[3.3]heptan-2-yl]- [2-(1, 1-dioxothietan-3- yl)sulfonyl-2,6- diazaspiro[3.3]heptan-6- yl]methanone | 497.4 [M + H]⁺ |
| 340 | 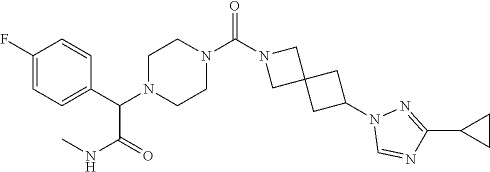 [SMILES: CNC(=O)C(c1ccc(F)cc1)N1CCN(C(=O)N2CC3(CC(n4cnc(C5CC5)n4)C3)C2)CC1] | 2-[4-[6-(3-cyclopropyl- 1,2,4-triazol-1-yl)-2- azaspiro[3.3]heptane-2- carbonyl]piperazin-1-yl]-2- (4-fluorophenyl)-N-methyl- acetamide | 482.4 [M + H]⁺ |
| 343 | 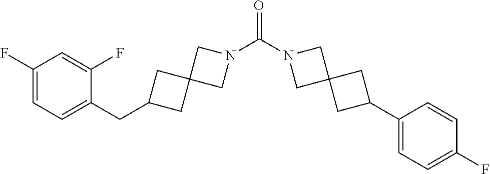 [SMILES: O=C(N1CC2(CC(Cc3ccc(F)cc3F)C2)C1)N1CC2(CC(c3ccc(F)cc3)C2)C1] | [6-[(2,4- difluorophenyl)methyl]-2- azaspiro[3.3]heptan-2-yl]- [6-(4-fluorophenyl)-2- azaspiro[3.3]heptan-2- yl]methanone | 441.2 [M + H]⁺ |
| 345 | 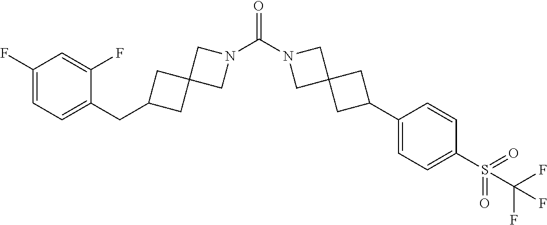 [SMILES: O=C(N1CC2(CC(Cc3ccc(F)cc3F)C2)C1)N1CC2(CC(c3ccc(S(=O)(=O)C(F)(F)F)cc3)C2)C1] | [6-[(2,4- difluorophenyl)methyl]-2- azaspiro[3.3]heptan-2-yl]- [6-[4- (trifluoromethylsulfonyl) phenyl]-2-azaspiro[3.3]heptan- 2-yl]methanone | 555.2 [M + H]⁺ |
| 346 | 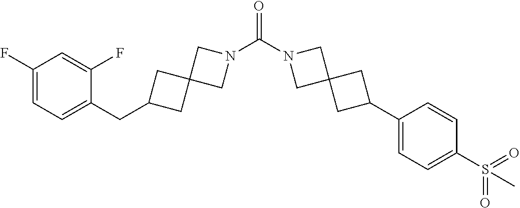 [SMILES: CS(=O)(=O)c1ccc(C2CC3(C2)CN(C(=O)N2CC4(CC(Cc5ccc(F)cc5F)C4)C2)C3)cc1] | [6-[(2,4- difluorophenyl)methyl]-2- azaspiro[3.3]heptan-2-yl]- [6-(4- methylsulfonylphenyl)-2- azaspiro[3.3]heptan-2- yl]methanone | 501.3 [M + H]⁺ |
| 347 | 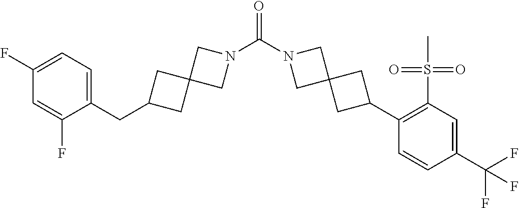 [SMILES: CS(=O)(=O)c1cc(C(F)(F)F)ccc1C1CC2(C1)CN(C(=O)N1CC3(CC(Cc4ccc(F)cc4F)C3)C1)C2] | [6-[(2,4- difluorophenyl)methyl]-2- azaspiro[3.3]heptan-2-yl]- [6-[2-methylsulfonyl-4- (trifluoromethyl)phenyl]-2- azaspiro[3.3]heptan-2- yl]methanone | 569.3 [M + H]⁺ |
| 354 | 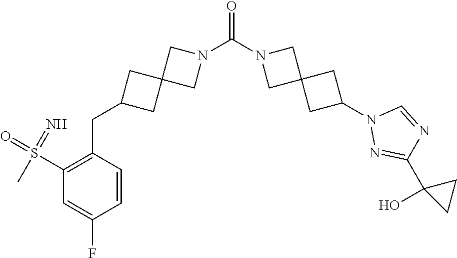 [SMILES: CS(=N)(=O)c1cc(F)ccc1CC1CC2(C1)CN(C(=O)N1CC3(CC(n4cnc(C5(O)CC5)n4)C3)C1)C2] | [6-[[4-fluoro-2- (methylsulfonimidoyl)phenyl] methyl]-2- azaspiro[3.3]heptan-2-yl]- [6-[3-(1- hydroxycyclopropyl)-1,2,4- triazol-1-yl]-2- azaspiro[3.3]heptan-2- yl]methanone | 529.3 [M + H]⁺ |
| 355 | 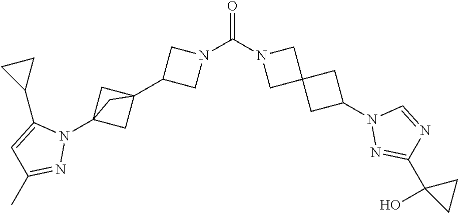 [SMILES: Cc1cc(C2CC2)n(C23CC(C4CN(C(=O)N5CC6(CC(n7cnc(C8(O)CC8)n7)C6)C5)C4)(C2)C3)n1] | [3-[3-(5-cyclopropyl-3- methyl-pyrazol-1-yl)-1- bicyclo[1.1.1]pentanyl] azetidin-1-yl]-[6-[3-(1- hydroxycyclopropyl)-1,2,4- triazol-1-yl]-2- azaspiro[3.3]heptan-2- yl]methanone | 490.4 [M + H]⁺ |
| 356 | 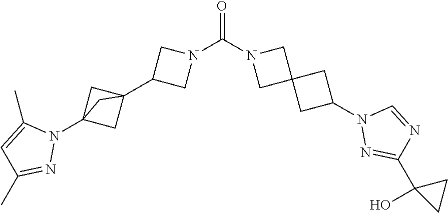 [SMILES: Cc1cc(C)n(C23CC(C4CN(C(=O)N5CC6(CC(n7cnc(C8(O)CC8)n7)C6)C5)C4)(C2)C3)n1] | [3-[3-(3,5-dimethylpyrazol- 1-yl)-1- bicyclo[1.1.1]pentanyl] azetidin-1-yl]-[6-[3-(1- hydroxycyclopropyl)-1,2,4- triazol-1-yl]-2- azaspiro[3.3]heptan-2- yl]methanone | 464.4 [M + H]⁺ |
| 357 | 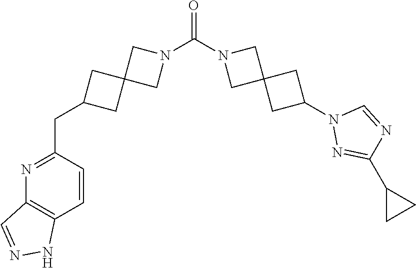 [SMILES: O=C(N1CC2(CC(Cc3ccc4[nH]ncc4n3)C2)C1)N1CC2(CC(n3cnc(C4CC4)n3)C2)C1] | [6-(3-cyclopropyl-1,2,4- triazol-1-yl)-2- azaspiro[3.3]heptan-2-yl]- [6-(1H-pyrazolo[4,3- b]pyridin-5-ylmethyl)-2- azaspiro[3.3]heptan-2- yl]methanone | 459.4 [M + H]⁺ |
| 359 | 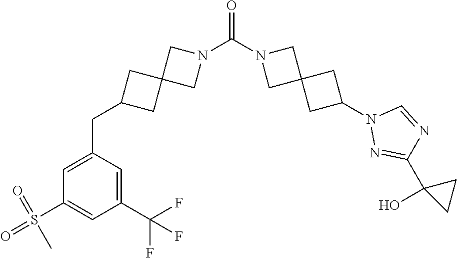 [SMILES: CS(=O)(=O)c1cc(CC2CC3(C2)CN(C(=O)N2CC4(CC(n5cnc(C6(O)CC6)n5)C4)C2)C3)cc(C(F)(F)F)c1] | [6-[3-(1- hydroxycyclopropyl)-1,2,4- triazol-1-yl]-2- azaspiro[3.3]heptan-2-yl]- [6-[[3-methylsulfonyl-5- (trifluoromethyl)phenyl] methyl]-2-azaspiro[3.3]heptan- 2-yl]methanone | 580.3 [M + H]⁺ |
| 360 | 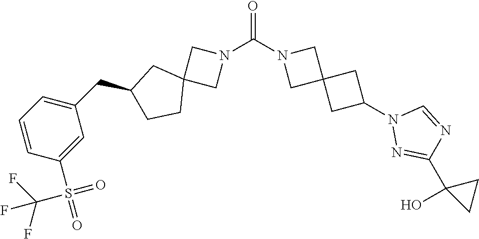 [SMILES: O=C(N1CC2(CC(n3cnc(C4(O)CC4)n3)C2)C1)N1CC2(CC[C@@H](Cc3cccc(S(=O)(=O)C(F)(F)F)c3)C2)C1] | [6-[3-(1- hydroxycyclopropyl)-1,2,4- triazol-1-yl]-2- azaspiro[3.3]heptan-2-yl]- [(6S)-6-[[3- (trifluoromethylsulfonyl) phenyl]methyl]-2- azaspiro[3.4]octan-2- yl]methanone | 580.3 [M + H]⁺ |
| 361 | 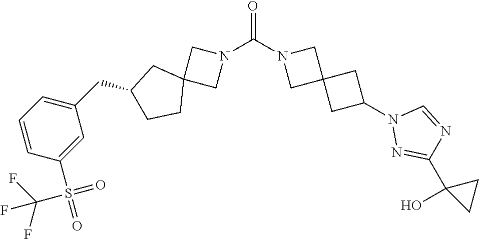 [SMILES: O=C(N1CC2(CC(n3cnc(C4(O)CC4)n3)C2)C1)N1CC2(CC[C@H](Cc3cccc(S(=O)(=O)C(F)(F)F)c3)C2)C1] | [6-[3-(1- hydroxycyclopropyl)-1,2,4- triazol-1-yl]-2- azaspiro[3.3]heptan-2-yl]- [(6R)-6-[[3- (trifluoromethylsulfonyl) phenyl]methyl]-2- azaspiro[3.4]octan-2- yl]methanone | 580.3 [M + H]⁺ |
| 362 | 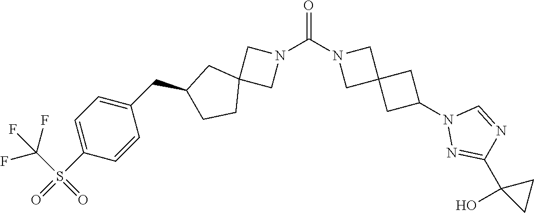 [SMILES: O=C(N1CC2(CC(n3cnc(C4(O)CC4)n3)C2)C1)N1CC2(CC[C@@H](Cc3ccc(S(=O)(=O)C(F)(F)F)cc3)C2)C1] | [6-[3-(1- hydroxycyclopropyl)-1,2,4- triazol-1-yl]-2- azaspiro[3.3]heptan-2-yl]- [(6S)-6-[[4- (trifluoromethylsulfonyl) phenyl]methyl]-2- azaspiro[3.4]octan-2- yl]methanone | 580.3 [M + H]⁺ |
| 363 | 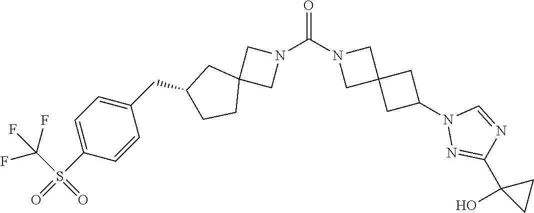 [SMILES: O=C(N1CC2(CC(n3cnc(C4(O)CC4)n3)C2)C1)N1CC2(CC[C@H](Cc3ccc(S(=O)(=O)C(F)(F)F)cc3)C2)C1] | [6-[3-(1- hydroxycyclopropyl)-1,2,4- triazol-1-yl]-2- azaspiro[3.3]heptan-2-yl]- [(6R)-6-[[4- (trifluoromethylsulfonyl) phenyl]methyl]-2- azaspiro[3.4]octan-2- yl]methanone | 580.3 [M + H]⁺ |
| 364 | 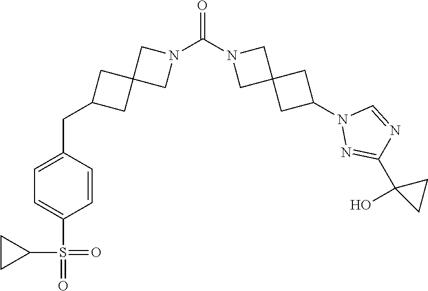 [SMILES: O=C(N1CC2(CC(Cc3ccc(S(=O)(=O)C4CC4)cc3)C2)C1)N1CC2(CC(n3cnc(C4(O)CC4)n3)C2)C1] | [6-[(4- cyclopropylsulfonylphenyl) methyl]-2- azaspiro[3.3]heptan-2-yl]- [6-[3-(1- hydroxycyclopropyl)-1,2,4- triazol-1-yl]-2- azaspiro[3.3]heptan-2- yl]methanone | 538.4 [M + H]⁺ |
| 369 | 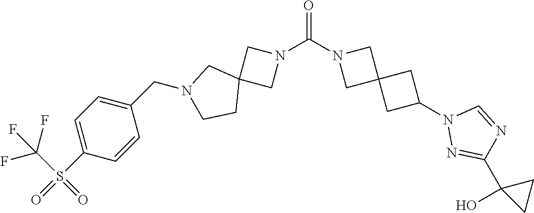 [SMILES: O=C(N1CC2(CC(n3cnc(C4(O)CC4)n3)C2)C1)N1CC2(CCN(Cc3ccc(S(=O)(=O)C(F)(F)F)cc3)C2)C1] | [6-[3-(1- hydroxycyclopropyl)-1,2,4- triazol-1-yl]-2- azaspiro[3.3]heptan-2-yl]- [7-[[4- (trifluoromethylsulfonyl) phenyl]methyl]-2,7- diazaspiro[3.4]octan-2- yl]methanone | 581.3 [M + H]⁺ |
| 382 | 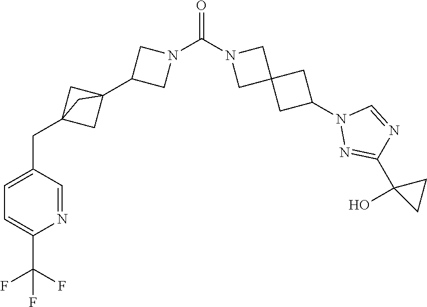 [SMILES: O=C(N1CC(C23CC(Cc4ccc(C(F)(F)F)nc4)(C2)C3)C1)N1CC2(CC(n3cnc(C4(O)CC4)n3)C2)C1] | [6-[3-(1- hydroxycyclopropyl)-1,2,4- triazol-1-yl]-2- azaspiro[3.3]heptan-2-yl]- [3-[3-[[6-(trifluoromethyl)- 3-pyridyl]methyl]-1- bicyclo[1.1.1]pentanyl] azetidin-1-yl]methanone | 529.4 [M + H]⁺ |
| 383 | 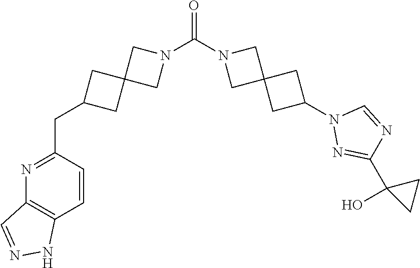 [SMILES: O=C(N1CC2(CC(Cc3ccc4[nH]ncc4n3)C2)C1)N1CC2(CC(n3cnc(C4(O)CC4)n3)C2)C1] | [6-[3-(1- hydroxycyclopropyl)-1,2,4- triazol-1-yl]-2- azaspiro[3.3]heptan-2-yl]- [6-(1H-pyrazolo[4,3- b]pyridin-5-ylmethyl)-2- azaspiro[3.3]heptan-2- yl]methanone | 475.4 [M + H]⁺ |
| 386 | 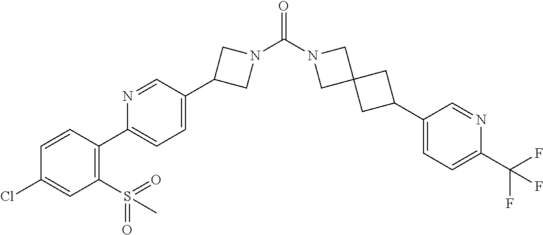 [SMILES: CS(=O)(=O)c1cc(Cl)ccc1-c1ccc(C2CN(C(=O)N3CC4(CC(c5ccc(C(F)(F)F)nc5)C4)C3)C2)cn1] | [3-[6-(4-chloro-2- methylsulfonyl-phenyl)-3- pyridyl]azetidin-1-yl]-[6-[6- (trifluoromethyl)-3-pyridyl]- 2-azaspiro[3.3]heptan-2- yl]methanone | 591.3 [M + H]⁺ |
| 394 | 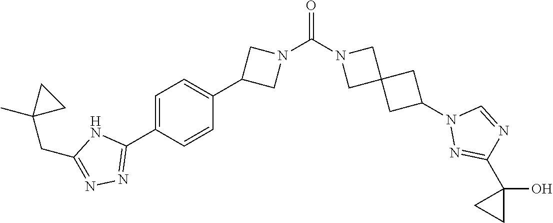 [SMILES: CC1(Cc2nnc(-c3ccc(C4CN(C(=O)N5CC6(CC(n7cnc(C8(O)CC8)n7)C6)C5)C4)cc3)[nH]2)CC1] | [6-[3-(1- hydroxycyclopropyl)-1,2,4- triazol-1-yl]-2- azaspiro[3.3]heptan-2-yl]- [3-[4-[5-[(1- methylcyclopropyl)methyl]- 4H-1,2,4-triazol-3- yl]phenyl]azetidin-1- yl]methanone | 515.5 [M + H]⁺ |
| 416 | 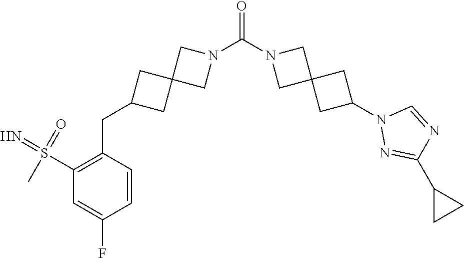 [SMILES: CS(=N)(=O)c1cc(F)ccc1CC1CC2(C1)CN(C(=O)N1CC3(CC(n4cnc(C5CC5)n4)C3)C1)C2] | [6-(3-cyclopropyl-1,2,4- triazol-1-yl)-2- azaspiro[3.3]heptan-2-yl]- [6-[[4-fluoro-2- (methylsulfonimidoyl)phenyl] methyl]-2- azaspiro[3.3]heptan-2- yl]methanone | 513.4 [M + H]⁺ |
| 425 | 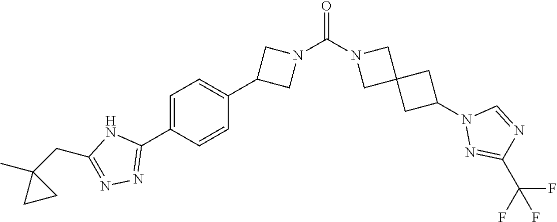 [SMILES: CC1(Cc2nnc(-c3ccc(C4CN(C(=O)N5CC6(CC(n7cnc(C(F)(F)F)n7)C6)C5)C4)cc3)[nH]2)CC1] | [3-[4-[5-[(1- methylcyclopropyl)methyl]- 4H-1,2,4-triazol-3- yl]phenyl]azetidin-1-yl]-[6- [3-(trifluoromethyl)-1,2,4- triazol-1-yl]-2- azaspiro[3.3]heptan-2- yl]methanone | 527.5 [M + H]⁺ |
| 428 | 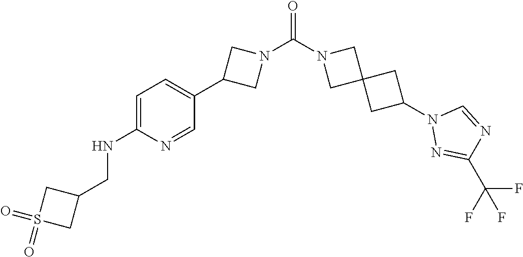 [SMILES: O=C(N1CC(c2ccc(NCC3CS(=O)(=O)C3)nc2)C1)N1CC2(CC(n3cnc(C(F)(F)F)n3)C2)C1] | [3-[6-[(1,1-dioxothietan-3- yl)methylamino]-3- pyridyl]azetidin-1-yl]-[6-[3- (trifluoromethyl)-1,2,4- triazol-1-yl]-2- azaspiro[3.3]heptan-2- yl]methanone | 526.4 [M + H]⁺ |
| 434 | 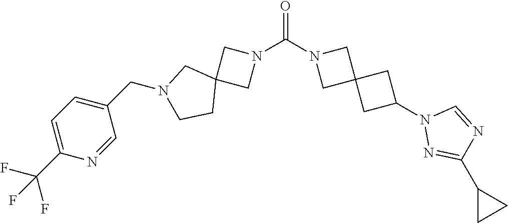 [SMILES: O=C(N1CC2(CC(n3cnc(C4CC4)n3)C2)C1)N1CC2(CCN(Cc3ccc(C(F)(F)F)nc3)C2)C1] | [6-(3-cyclopropyl-1,2,4- triazol-1-yl)-2- azaspiro[3.3]heptan-2-yl]- [7-[6-(trifluoromethyl)-3- pyridyl]methyl]-2,7- diazaspiro[3.4]octan-2- yl]methanone | 502.5 [M + H]⁺ |
| 448 | 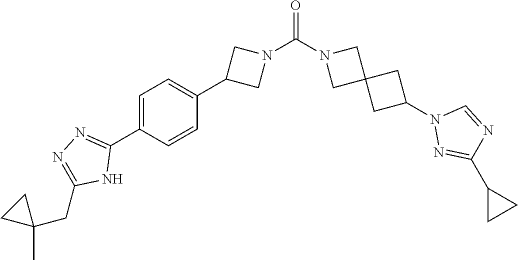 [SMILES: CC1(Cc2nnc(-c3ccc(C4CN(C(=O)N5CC6(CC(n7cnc(C8CC8)n7)C6)C5)C4)cc3)[nH]2)CC1] | [6-(3-cyclopropyl-1,2,4- triazol-1-yl)-2- azaspiro[3.3]heptan-2-yl]- [3-[4-[5-[(1- methylcyclopropyl)methyl]- 4H-1,2,4-triazol-3- yl]phenyl]azetidin-1- yl]methanone | 499.4 [M + H]⁺ |
| 450 | 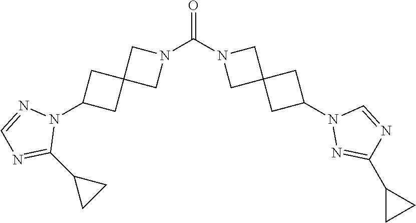 [SMILES: O=C(N1CC2(CC(n3cnc(C4CC4)n3)C2)C1)N1CC2(CC(n3ncnc3C3CC3)C2)C1] | [6-(3-cyclopropyl-1,2,4- triazol-1-yl)-2- azaspiro[3.3]heptan-2-yl]- [6-(5-cyclopropyl-1,2,4- triazol-1-yl)-2- azaspiro[3.3]heptan-2- yl]methanone | 435.4 [M + H]⁺ |
| 453 | 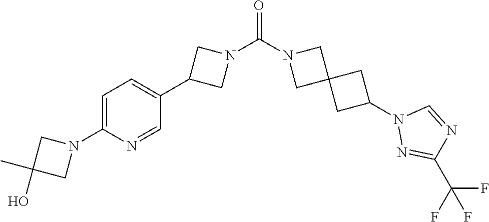 [SMILES: CC1(O)CN(c2ccc(C3CN(C(=O)N4CC5(CC(n6cnc(C(F)(F)F)n6)C5)C4)C3)cn2)C1] | [3-[6-(3-hydroxy-3-methyl- azetidin-1-yl)-3- pyridyl]azetidin-1-yl]-[6-[3- (trifluoromethyl)-1,2,4- triazol-1-yl]-2- azaspiro[3.3]heptan-2- yl]methanone | 478.4 [M + H]⁺ |
| 467 | 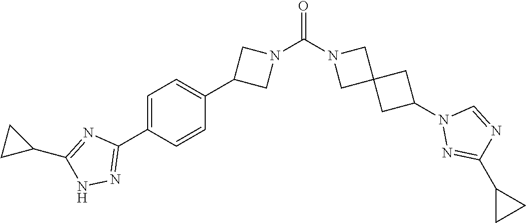 [SMILES: O=C(N1CC(c2ccc(-c3n[nH]c(C4CC4)n3)cc2)C1)N1CC2(CC(n3cnc(C4CC4)n3)C2)C1] | [6-(3-cyclopropyl-1,2,4- triazol-1-yl)-2- azaspiro[3.3]heptan-2-yl]- [3-[4-(5-cyclopropyl-1H- 1,2,4-triazol-3- yl)phenyl]azetidin-1- yl]methanone | 471.4 [M + H]⁺ |
| 471 | 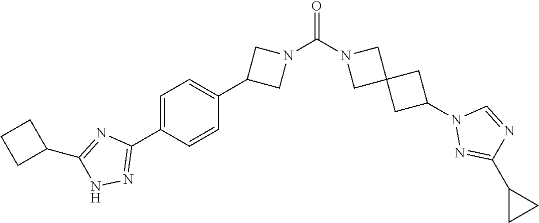 [SMILES: O=C(N1CC(c2ccc(-c3n[nH]c(C4CCC4)n3)cc2)C1)N1CC2(CC(n3cnc(C4CC4)n3)C2)C1] | [3-[4-(5-cyclobutyl-1H- 1,2,4-triazol-3- yl)phenyl]azetidin-1-yl]-[6- (3-cyclopropyl-1,2,4-triazol- 1-yl)-2-azaspiro[3.3]heptan- 2-yl]methanone | 485.3 [M + H]⁺ |
| 504 | 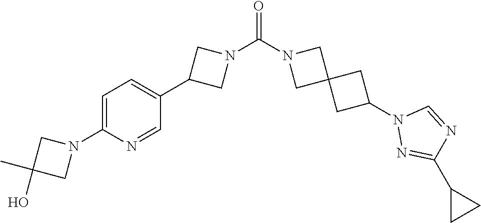 [SMILES: CC1(O)CN(c2ccc(C3CN(C(=O)N4CC5(CC(n6cnc(C7CC7)n6)C5)C4)C3)cn2)C1] | [6-(3-cyclopropyl-1,2,4- triazol-1-yl)-2- azaspiro[3.3]heptan-2-yl]- [3-[6-(3-hydroxy-3-methyl- azetidin-1-yl)-3- pyridyl]azetidin-1- yl]methanone | 450.3 [M + H]⁺ |
| 505 | 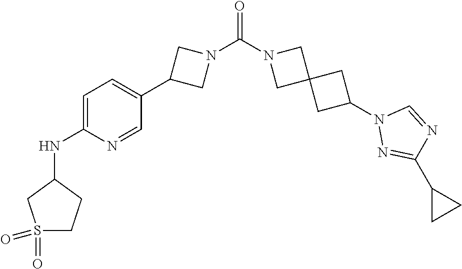 [SMILES: O=C(N1CC(c2ccc(NC3CCS(=O)(=O)C3)nc2)C1)N1CC2(CC(n3cnc(C4CC4)n3)C2)C1] | [6-(3-cyclopropyl-1,2,4- triazol-1-yl)-2- azaspiro[3.3]heptan-2-yl]- [3-[6-[(1,1-dioxothiolan-3- yl)amino]-3- pyridyl]azetidin-1- yl]methanone | 498.3 [M + H]⁺ |
| 506 | 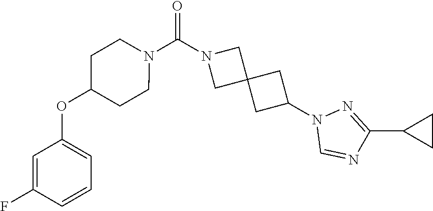 [SMILES: O=C(N1CCC(Oc2cccc(F)c2)CC1)N1CC2(CC(n3cnc(C4CC4)n3)C2)C1] | [6-(3-cyclopropyl-1,2,4- triazol-1-yl)-2- azaspiro[3.3]heptan-2-yl]- [4-(3-fluorophenoxy)-1- piperidyl]methanone | 426.4 [M + H]⁺ |
| 507 | 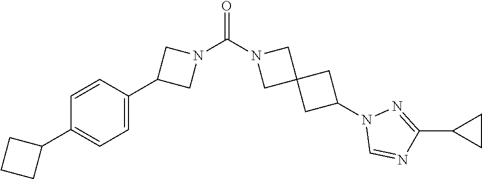 [SMILES: O=C(N1CC(c2ccc(C3CCC3)cc2)C1)N1CC2(CC(n3cnc(C4CC4)n3)C2)C1] | [3-(4- cyclobutylphenyl)azetidin- 1-yl]-[6-(3-cyclopropyl- 1,2,4-triazol-1-yl)-2- azaspiro[3.3]heptan-2- yl]methanone | 418.3 [M + H]⁺ |
| 508 | 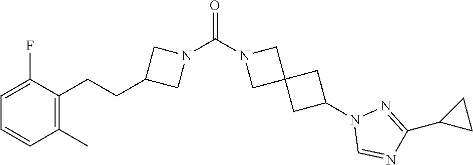 [SMILES: Cc1cccc(F)c1CCC1CN(C(=O)N2CC3(CC(n4cnc(C5CC5)n4)C3)C2)C1] | [6-(3-cyclopropyl-1,2,4- triazol-1-yl)-2- azaspiro[3.3]heptan-2-yl]- [3-[2-(2-fluoro-6-methyl- phenyl)ethyl]azetidin-1- yl]methanone | 424.2 [M + H]⁺ |
| 509 | 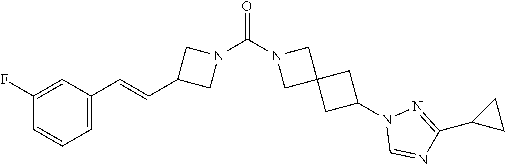 [SMILES: O=C(N1CC(/C=C/c2cccc(F)c2)C1)N1CC2(CC(n3cnc(C4CC4)n3)C2)C1] | [6-(3-cyclopropyl-1,2,4- triazol-1-yl)-2- azaspiro[3.3]heptan-2-yl]- [3-[(E)-2-(3- fluorophenyl)vinyl]azetidin- 1-yl]methanone | 408.2 [M + H]⁺ |
| 511 | 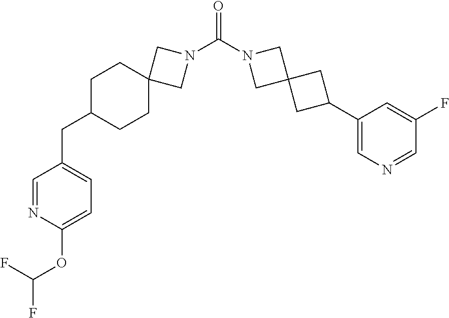 [SMILES: O=C(N1CC2(CCC(Cc3ccc(OC(F)F)nc3)CC2)C1)N1CC2(CC(c3cncc(F)c3)C2)C1] | [7-[[6-(difluoromethoxy)-3- pyridyl]methyl]-2- azaspiro[3.5]nonan-2-yl]-[6- (5-fluoro-3-pyridyl)-2- azaspiro[3.3]heptan-2- yl]methanone | 501.4 [M + H]⁺ |
| 521 | 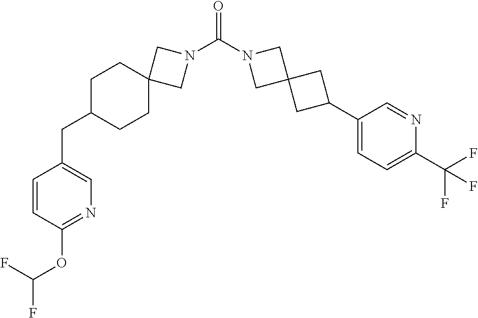 [SMILES: O=C(N1CC2(CCC(Cc3ccc(OC(F)F)nc3)CC2)C1)N1CC2(CC(c3ccc(C(F)(F)F)nc3)C2)C1] | [7-[[6-(difluoromethoxy)-3- pyridyl]methyl]-2- azaspiro[3.5]nonan-2-yl]-[6- [6-(trifluoromethyl)-3- pyridyl]-2- azaspiro[3.3]heptan-2- yl]methanone | 551.4 [M + H]⁺ |
| 522 | 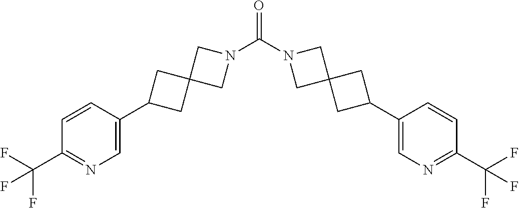 [SMILES: O=C(N1CC2(CC(c3ccc(C(F)(F)F)nc3)C2)C1)N1CC2(CC(c3ccc(C(F)(F)F)nc3)C2)C1] | bis[6-[6-(trifluoromethyl)-3- pyridyl]-2- azaspiro[3.3]heptan-2- yl]methanone | 511.4 [M + H]⁺ |
| 537 | 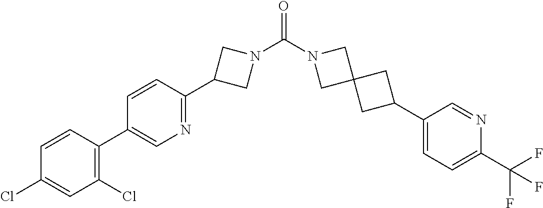 [SMILES: O=C(N1CC(c2ccc(-c3ccc(Cl)cc3Cl)cn2)C1)N1CC2(CC(c3ccc(C(F)(F)F)nc3)C2)C1] | [3-[5-(2,4-dichlorophenyl)- 2-pyridyl]azetidin-1-yl]-[6- [6-(trifluoromethyl)-3- pyridyl]-2- azaspiro[3.3]heptan-2- yl]methanone | 547.3 [M + H]⁺ |
| 538 | 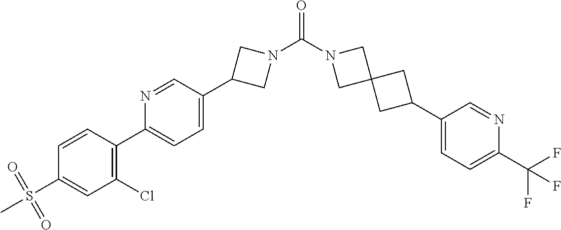 [SMILES: CS(=O)(=O)c1ccc(-c2ccc(C3CN(C(=O)N4CC5(CC(c6ccc(C(F)(F)F)nc6)C5)C4)C3)cn2)c(Cl)c1] | [3-[6-(2-chloro-4- methylsulfonyl-phenyl)-3- pyridyl]azetidin-1-yl]-[6-[6- (trifluoromethyl)-3-pyridyl]- 2-azaspiro[3.3]heptan-2- yl]methanone | 591.3 [M + H]⁺ |
| 539 | 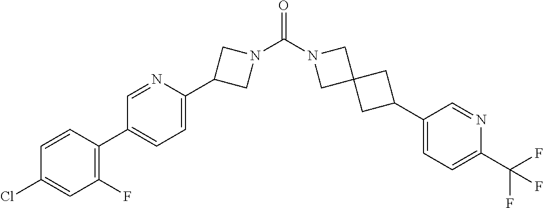 [SMILES: O=C(N1CC(c2ccc(-c3ccc(Cl)cc3F)cn2)C1)N1CC2(CC(c3ccc(C(F)(F)F)nc3)C2)C1] | [3-[5-(4-chloro-2-fluoro- phenyl)-2-pyridyl]azetidin- 1-yl]-[6-[6- (trifluoromethyl)-3-pyridyl]- 2-azaspiro[3.3]heptan-2- yl]methanone | 531.3 [M + H]⁺ |

### Synthesis of Building Blocks

### Example A.1

### 6-(3-cyclopropyl-1H-1,2,4-triazol-1-yl)-2-azaspiro[3.3]heptane 2,2,2-trifluoroacetate

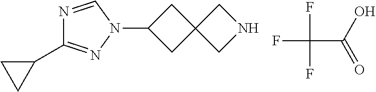

To a solution of tert-butyl 6-(3-cyclopropyl-1,2,4-triazol-1-yl)-2-azaspiro[3.3]heptane-2-carboxylate (6.00 g, 19.7 mmol) in DCM (120 mL) was added TFA (46.2 g, 405 mmol, 30 mL) at 25° C. The mixture was stirred at 30° C. for 16 h, before being evaporated. The title compound (14.0 g, crude) was used in the next step without further purification. MS (ESI): m/z=205.2 [M+H]⁺

### Step a) tert-butyl 6-methylsulfonyloxy-2-azaspiro[3.3]heptane-2-carboxylate

To a solution of tert-butyl 6-hydroxy-2-azaspiro[3.3]heptane-2-carboxylate (CAS: 1147557-97-8) (10.0 g, 46.9 mmol) in DCM (200 mL) was added TEA (9.79 mL, 70.3 mmol) and MsCl (4.66 mL, 60.2 mmol) dropwise at 0° C. The mixture was stirred at 30° C. for 2 h. The reaction mixture was quenched by addition of aq NaHCO₃ solution (200 mL), and then extracted with DCM (300 mL×2). The combined organic layers were dried over Na₂SO₄, filtered and evaporated to give the title compound (13.5 g crude, 98.8% yield), which was used into the next step without further purification. MS (ESI): m/z=236.2 [M+H]⁺

### Step b) tert-butyl 6-(3-cyclopropyl-1,2,4-triazol-1-yl)-2-azaspiro[3.3]heptane-2-carboxylate

To a solution of tert-butyl 6-methylsulfonyloxy-2-azaspiro[3.3]heptane-2-carboxylate (12.0 g, 41.2 mmol, 90.0% purity) in ACN (200 mL) was added 3-cyclopropyl-1H-1,2,4-triazole (CAS: 1211390-33-8) (4.50 g, 41.2 mmol) and Cs₂CO₃ (26.8 g, 82.4 mmol) at 25° C. The mixture was stirred at 100° C. for 16 h. The reaction mixture was filtered and evaporated. The residue was further separated by SFC to obtain the title compound (6.77 g, 54.0% yield) as a brown solid. MS (ESI): m/z=305.2 [M+H]⁺

In analogy to Example A.1, Examples in the following table were generated using the respective heteroarene building blocks.

| | | | | MS, |
|---|---|---|---|---|
| | | | Building | ESI: |
| Ex. | Structure | Systematic Name | Blocks | m/z |
| A.2 | 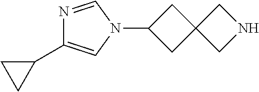 [SMILES: c1nc(C2CC2)cn1C1CC2(CNC2)C1] | 6-(4- cyclopropylimidazol- 1-yl)-2- azaspiro[3.3]heptane; 2,2,2-trifluoroacetic acid salt | 5- cyclopropyl- 1H- imidazole (CAS: 89830-98-8) | 204.2 [M + H]⁺ |
| | 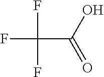 | | | |

### Example A.3

### 6-(5-cyclopropylpyrazol-1-yl)-2-azaspiro[3.3]heptane; 2,2,2-trifluoroacetic acid

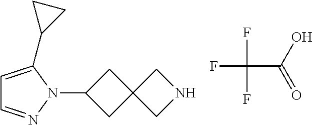

To a solution of tert-butyl 6-(5-cyclopropylpyrazol-1-yl)-2-azaspiro[3.3]heptane-2-carboxylate (40.0 mg, 0.13 mmol) in DCM (1 mL) was added trifluoroacetic acid (0.11 mL, 1.39 mmol) at 20° C. for 12 h. The mixture was evaporated, to give the crude title compound (40.0 mg, 96% yield) as light yellow oil. MS (ESI): m/z=204.7 [M+H]⁺

### Step a) tert-butyl 6-(5-cyclopropylpyrazol-1-yl)-2-azaspiro[3.3]heptane-2-carboxylate

To a solution of 3-cyclopropyl-1H-pyrazole (111 mg, 1.03 mmol), cesium carbonate (671 mg, 2.06 mmol) in DMF (15 mL) was added tert-butyl 6-methylsulfonyloxy-2-azaspiro[3.3]heptane-2-carboxylate (300 mg, 1.03 mmol), then stirred at 100° C. for 12 h. The reaction mixture was evaporated, the residue was purified by prep-HPLC (FA) and lyophilized to give the title compound (minor regioisomer) (40.0 mg, 13% yield) as a white solid. MS (ESI): m/z=248.6 [M−tBu+H]⁺

Regioisomeric products of several other building blocks could also be concurrently produced in a similar manner.

### Example A.4

### 6-(4-methylpyrazol-1-yl)-2-azaspiro[3.3]heptane; 2,2,2-trifluoroacetic acid salt

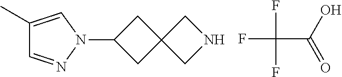

To a solution of trifluoroacetic acid (0.5 mL, 6.49 mmol) in DCM (2.5 mL) was added tert-butyl 6-(4-methylpyrazol-1-yl)-2-azaspiro[3.3]heptane-2-carboxylate (150 mg, 0.540 mmol), then stirred at 20° C. for 12 h. The mixture was evaporated to give the crude title compound (150 mg, 0.510 mmol, 95.2% yield) as light brown oil, which was used directly without further purification. MS (ESI): m/z=178.8 [M+H]⁺

### Step a) tert-butyl 6-(4-methylpyrazol-1-yl)-2-azaspiro[3.3]heptane-2-carboxylate

To a solution of 4-methyl pyrazole (84.5 mg, 1.03 mmol), cesium carbonate (671 mg, 2.06 mmol) in DMF (10 mL) was added tert-butyl 6-methylsulfonyloxy-2-azaspiro[3.3]heptane-2-carboxylate (CAS: 1239320-11-6) (300 mg, 1.03 mmol), then stirred at 100° C. for 12 h. The reaction mixture was concentrated in vacuum, the residue was purified by prep-HPLC (FA) and lyophilized to give the title compound (150 mg, 0.540 mmol, 52.5% yield) as white solid. MS (ESI): m/z=278.7 [M+H]⁺

In analogy to Example A.4, the following Examples were generated from the specified building blocks.

| | | | | MS, |
|---|---|---|---|---|
| | | | Building | ESI: |
| Ex. | Structure | Systematic Name | Blocks | m/z |
| A.5 | 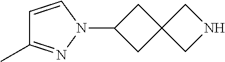 | 6-(3-methylpyrazol-1- yl)-2- azaspiro[3.3]heptane; 2,2,2-trifluoroacetic | 3- methylpyrazole (CAS: 1453- 58-3) | 178.8 [M + H]⁺ |
| | 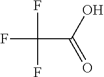 | acid salt | | |
| A.6 | 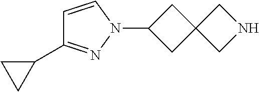 [SMILES: c1cn(C2CC3(CNC3)C2)nc1C1CC1] | 6-(3- cyclopropylpyrazol-1- yl)-2- azaspiro[3.3]heptane; 2,2,2-trifluoroacetic acid salt | 3-cyclopropyl- 1H-pyrazole (CAS: 100114- 57-6) | 204.7 [M + H]⁺ |
| | 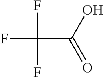 | | | |
| A.7 | 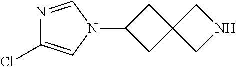 [SMILES: Clc1cn(C2CC3(CNC3)C2)cn1] | 6-(4-chloroimidazol-1- yl)-2- azaspiro[3.3]heptane; 2,2,2-trifluoroacetic acid | 4-chloro-1H- imidazole (CAS: 15965- 31-8) | 298.2 [M + H]⁺ (for Boc- inter- |
| | 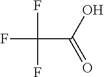 | | | mediate) |
| A.8 | 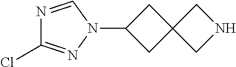 [SMILES: Clc1ncn(C2CC3(CNC3)C2)n1] | 6-(3-chloro-1,2,4- triazol-1-yl)-2- azaspiro[3.3]heptane; 2,2,2-trifluoroacetic acid | 3-chloro-1H- 1,2,4-triazole (CAS: 6818- 99-1) | 199.7 [M + H]⁺ |
| | 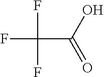 | | | |
| A.9 | 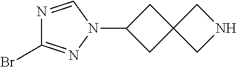 [SMILES: Brc1ncn(C2CC3(CNC3)C2)n1] | 6-(3-bromo-1,2,4- triazol-1-yl)-2- azaspiro[3.3]heptane; 2,2,2-trifluoroacetic acid | 3-bromo-1H- 1,2,4-triazole (CAS: 57704- 26-4) | 245.2 [M + H]⁺ |
| | 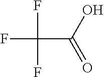 | | | |
| A.13 | 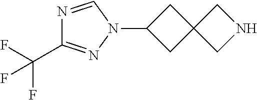 [SMILES: FC(F)(F)c1ncn(C2CC3(CNC3)C2)n1] | 2,2,2-trifluoroacetic acid;6-[3- (trifluoromethyl)-1,2,4- triazol-1-yl]-2- azaspiro[3.3]heptane | 3-(trifluoro- methyl)- 1H-1,2,4- triazole (CAS: 60406-75-9) | 233.1 [M + H]⁺ |
| | 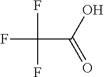 | | | |
| A.14 | 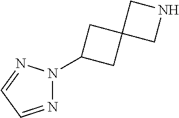 [SMILES: c1cnn(C2CC3(CNC3)C2)n1] | 4- methylbenzenesulfonic acid;6-(triazol-2-yl)-2- azaspiro[3.3]heptane | 2H-triazole (CAS: 288-35- 7) | 165.1 [M + H]⁺ |
| | 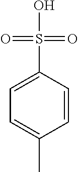 | | | |
| A.15 | 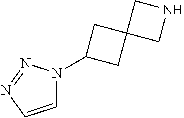 [SMILES: c1cn(C2CC3(CNC3)C2)nn1] | 4- methylbenzenesulfonic acid;6-(triazol-1-yl)-2- azaspiro[3.3]heptane | 2H-triazole (CAS: 288-35- 7) | 165.1 [M + H]⁺ |
| | 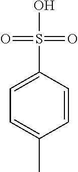 | | | |
| A.17 | 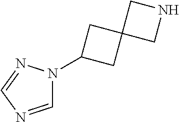 [SMILES: c1ncn(C2CC3(CNC3)C2)n1] | 4- methylbenzenesulfonic acid;6-(1,2,4-triazol-1- yl)-2- azaspiro[3.3]heptane | 1H-1,2,4- triazole (CAS: 288-88-0) | 165.1 [M + H]⁺ |
| | 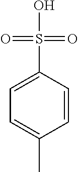 | | | |

### Example A.10

### 1-[l-(2-azaspiro1-[3.3] heptan-6-yl)-1,2,4-triazol-3-yl]cyclopropanol;2,2,2-trifluoroacetic acid

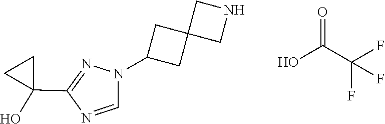 [SMILES: OC1(c2ncn(C3CC4(CNC4)C3)n2)CC1 ; O=C(O)C(F)(F)F]

To a solution of 6-[3-(1-hydroxycyclopropyl)-1,2,4-triazol-1-yl]-2-azaspiro[3.3]heptane-2-carboxylic acid tert-butyl ester (6.23 g, 19.06 mmol) in dichloromethane (80 mL) was added TFA (14.68 mL, 190.56 mmol) and the reaction mixture was stirred at RT for 18 h. Volatiles were removed in vacuo to give the title compound (12.68 g, quant.) as a crude light yellow viscous oil. MS (ESI): m/z=221.2 [M+H]⁺

### Step a) tert-butyl 6-(methylsulfonyloxy)-2-azaspiro[3.3]heptane-2-carboxylate

To a solution of tert-butyl 6-hydroxy-2-azaspiro[3.3]heptane-2-carboxylate (5.0 g, 23.4 mmol) in DCM (11.7 mL) was added TEA (6.54 mL, 46.9 mmol) followed by slow addition of methanesulfonyl chloride (2 mL, 25.8 mmol).The mixture was then stirred at RT for 17 h. The mixture was diluted with DCM (50 mL) and washed with water (50 mL). The aqueous phase was extracted with dichloromethane (2×50 mL). The combined organic phases were washed with brine (100 mL) dried over MgSO₄ and evaporated to dryness, to give the title compound (6.85 g, 98%) as a yellow solid. MS (ESI): m/z=236.2 [M−tBu+H]⁺

### Step b) 1-benzoxycyclopropanecarboxamide

To a solution of 1-benzoxycyclopropanecarboxylic acid (5.0 g, 26.01 mmol) in DCM (80 mL) cooled down to 0° C. was added CDI (4.43 g, 27.31 mmol) and the reaction mixture was stirred at 0° C. for 15 min and at r.t for 1 h. Addition of 2 M ammonia in iPrOH (32.52 mL, 65.03 mmol) to the reaction mixture which was then stirred at RT for 18 h. The reaction mixture was diluted with dichloromethane, poured into a separating funnel containing aqueous 1 M solution Na₂CO₃ for extraction. The organic phase was collected and the aqueous phase was back-extracted with dichloromethane. The combined organic phases were dried over sodium sulfate, filtered and evaporated, to give the title compound (5.05 g, 95% yield) as a crude brownish oil. MS (ESI): m/z=192.1 [M+H]⁺

### Step c) 3-(1-benzoxycyclopropyl)-]H-1,2,4-triazole

A solution of 1-benzoxycyclopropanecarboxamide (1.25 g, 6.41 mmol) in DMF-DMA (17.15 mL, 128.12 mmol) was stirred at 90° C. for 2.5 h, before being cooled down and evaporated. The residue was dissolved in 1,4-dioxane (15 mL) followed by addition of hydrazine 35% aqueous solution (1.15 mL, 12.81 mmol) and acetic acid (733.41 μL, 12.81 mmol) after which the reaction mixture was stirred at 90° C. for 18 h. The reaction mixture was poured into a separating funnel containing ethyl acetate and saturated aqueous NH₄Cl solution. After extraction, the organic phase was collected and the aqueous phase was back-extacted with ethyl acetate. The combined organic phases were dried over sodium sulfate and evaporated down to dryness to give the crude title compound (1.43 g, 98% yield). MS (ESI): m/z=216.1 [M+H]⁺

### Step d) 6-[3-(1-benzoxycyclopropyl)-1,2,4-triazol-1-yl]-2-azaspiro[3.3]heptane-2-carboxylic acid tert-butyl ester

To a solution of 3-(1-benzoxycyclopropyl)-1H-1,2,4-triazole (1.40 g, 6.18 mmol) in N,N-dimethylformamide (28 mL) cooled down to 0° C. was added NaH (259.51 mg, 6.49 mmol) and the reaction mixture was stirred at 0° C. for 10 min followed by addition of 6-methylsulfonyloxy-2-azaspiro[3.3]heptane-2-carboxylic acid tert-butyl ester (1.89 g, 6.49 mmol) after which the reaction mixture was stirred at 90° C. for 18 h. Volatiles were removed in vacuo, and the obtained crude residue was dissolved in ethyl acetate and poured into a separating funnel containing 1 M aqueous solution Na₂CO₃ for extraction. The organic phase was collected and the aqueous phase was back-extracted with ethyl acetate. The combined organic phases were dried over sodium sulfate and evaporated. Purification by FC(SiO₂; heptane/EtOAc) gave the title compound (1.41 g, 53%). MS (ESI): m/z=411.3 [M+H]⁺

### Step e): 6-[3-(1-hydroxycyclopropyl)-1,2,4-triazol-1-yl]-2-azaspiro[3.3]heptane-2-carboxylic acid tert-butyl ester

A solution of 6-[3-(1-benzoxycyclopropyl)-1,2,4-triazol-1-yl]-2-azaspiro[3.3]heptane-2-carboxylic acid tert-butyl ester (10.3 g, 25.1 mmol) in THF (206 mL) in an autoclave was put under an inert atmosphere by vacuum evacuation and Argon backfill after which 5% Pd/C (JM Type 424 (5R⁴²⁴), wet (59.1% H₂O), Het-131-1) (2 g) was added and the autoclave was sealed. The atmosphere was changed for hydrogen and the autoclave was put under a pressure of 2 bars H₂ after which the reaction mixture was stirred at 50° C. for 6 h. Reaction control indicated partial conversion to the desired product but some starting material still present. After addition of 5% Pd/C (JM Type 424 (5R⁴²⁴), wet (59.1% H₂O), Het-131-1) (1 g), the same procedure described above was carried out and the reaction mixture was stirred at 50° C. for another 6 h under a pressure of 2 bar H₂. At the end of the reaction, autoclave atmosphere was switched to an inert atmosphere and Pd catalyst was removed by filtration over a pad of Celite. The collected filtrate was concentrated in vacuo to give 7.92 g of the crude desired product. Purification by FC (SiO₂; heptane/EtOAc/EtOH) gave the title compound as a white solid. MS (ESI): m/z=321.2 [M+H]⁺

### Example A.11

### 6-[6-(trifluoromethyl)-3-pyridyl]-2-azaspiro[3.3]heptane; 4-methylbenzenesulfonic acid

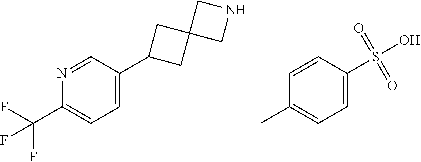 [SMILES: FC(F)(F)c1ccc(C2CC3(CNC3)C2)cn1 ; Cc1ccc(S(=O)(=O)O)cc1]

The mixture of tert-butyl 6-[6-(trifluoromethyl)-3-pyridyl]-2-azaspiro[3.3]heptane-2-carboxylate (1450 mg, 4.24 mmol) and p-toluenesulfonic acid (1605 mg, 9.32 mmol) in ethyl acetate (10 mL) was stirred at 80° C. for 16 h. The reaction mixture was filtered and the cake was concentrated to give the title compound (2110 mg, 3.6 mmol, 84% yield) as an off-white solid. MS (ESI): m/z=243.3 [M-TsOH+H]⁺

### Step a) tert-butyl 6-[6-(trifluoromethyl)-3-pyridyl]-2-azaspiro[3.3]heptane-2-carboxylate

Two batches were set up in parallel. Tert-butyl 6-iodo-2-azaspiro[3.3]heptane-2-carboxylate (1.0 g, 3.09 mmol), 2-trifluoromethylpyridine-5-boronic acid (1180 mg, 6.19 mmol), Sodium bis(trimethylsilyl)amide in THF (6.19 mL, 6.19 mmol), trans-2-aminocyclohexanol hydrochloride (28.2 mg, 0.190 mmol) and nickel(II) iodide (58.0 mg, 0.190 mmol) were taken up into a microwave tube in 2-propanol (10 mL).The sealed tube was heated at 110° C. for 2.5 h under microwave. The reaction was quenched by H₂O slowly. The residue was purified by flash silica gel chromatography (eluent of 0 to 20% ethyl acetate/petroleum ether gradient) to give the title compound (1.5 g, 4.38 mmol, 71% yield) as a yellow solid. MS (ESI): m/z=287.2 [M−tBu+H]⁺

In analogy to Example A.11, the following Examples were generated from the specified building blocks.

| | | | | MS, |
|---|---|---|---|---|
| | | | Building | ESI: |
| Ex. | Structure | Systematic Name | Blocks | m/z |
| A.12 | 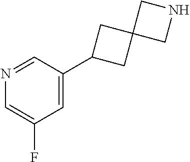 [SMILES: Fc1cncc(C2CC3(CNC3)C2)c1] | 6-(5-fluoro-3-pyridyl)- 2- azaspiro[3.3]heptane;4- methylbenzenesulfonic acid | Tert-butyl 6- iodo-2- azaspiro[3.3] heptane-2- carboxylate; (5-fluoro-3- pyridyl) boronic acid | 193.2 [M + H]⁺ |
| | 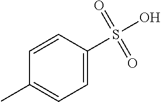 | | | |

### Example A.16

### 4-methylbenzenesulfonic acid;6-[2-(trifluoromethyl)pyrimidin-5-yl]-2-azaspiro[3.3]heptane

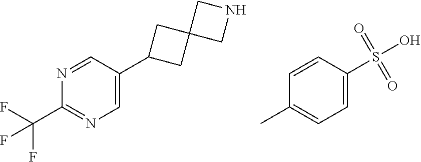 [SMILES: FC(F)(F)c1ncc(C2CC3(CNC3)C2)cn1 ; Cc1ccc(S(=O)(=O)O)cc1]

A solution of tert-butyl 6-[2-(trifluoromethyl)pyrimidin-5-yl]-2-azaspiro[3.3]heptane-2-carboxylate (1100.0 mg, 3.2 mmol) and p-toluenesulfonic acid (662.04 mg, 3.84 mmol) in EtOAc (10 mL) was stirred for 16 h at 80° C., before being evaporated. The residue was treated with deionized water and lyophilized, to give the title compound (1.3 g, 94.2%) as a white solid. MS (ESI): m/z=244.0 [M-TsOH+H]⁺

### Step a): tert-butyl 6-[2-(trifluoromethyl)pyrimidin-5-yl]-2-azaspiro[3.3]heptane-2-carboxylate

To a solution of tert-butyl 6-iodo-2-azaspiro[3.3]heptane-2-carboxylate (3.1 g, 9.69 mmol), 5-bromo-2-(trifluoromethyl)pyrimidine (1.1 g, 4.85 mmol), Ir[dF(CF₃)ppy]₂(dtbpy)(PF₆) (54.33 mg, 0.050 mmol), NiCl₂·dtbbpy (9.64 mg, 0.020 mmol), TTMSS (1.2 g, 4.85 mmol), Na₂CO₃ (1.0 g, 9.69 mmol) in DME (20 mL).The vial was sealed and placed under nitrogen. The reaction was stirred and irradiated with a 34 W blue LED lamp (7 cm away) equipped with a cooling fan (25° C.) for 14 h. Purification by FC (PE/EtOAc) and RP-HPLC gave the title compound (1.1 g, 66.11% yield) as white solid. MS (ESI): m/z=288.1, [M-C₄H₈+H]⁺

### Example A.18

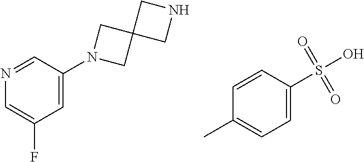

A solution of tert-butyl 6-(5-fluoro-3-pyridyl)-2,6-diazaspiro[3.3]heptane-2-carboxylate (500.0 mg, 1.7 mmol) in EtOAc (40 mL) was treated with p-toluenesulfonic acid monohydrate (713.31 mg, 3.75 mmol), at 23° C. The mixture was heated to 47° C., and stirred for 18 h at this temperature, before being evaporated. Trituration with Et₂O gave the title compound (742.4 mg, 76.96% yield) as a white solid. MS (ESI): m/z=194 [M+H]⁺

### Step a): tert-butyl 6-(5-fluoro-3-pyridyl)-2,6-diazaspiro[3.3]heptane-2-carboxylate

3-Bromo-5-fluoropyridine (0.4 g, 2.27 mmol), 9,9-dimethyl-4,5-bis(diphenylphosphino)xanthene (65.76 mg, 0.110

mmol), tris(dibenzylideneacetone)dipalladium (0) (104.06 mg, 0.110 mmol), tert-butyl 2,6-diazaspiro[3.3]heptane-2-carboxylate hydrochl (1.07 g, 4.55 mmol), cesium carbonate (2.96 g, 9.09 mmol) and 9,9-dimethyl-4,5-bis(diphenylphosphino)xanthene (65.76 mg, 0.110 mmol) were charged in a sealed tube and treated with 1,4-dioxane (60 mL). The mixture was sparged with Ar and stirred for 18 h at 100° C., before being evaporated. Purification by FC (hexane/MTBE) gave the title (450 mg, 67.49% yield) as a white solid. MS (ESI): m/z=294.0 [M+H]⁺

### Example B.1

### 3-(4-(1-(trifluoromethyl)cyclopropyl)phenyl)azetidine 4-methylbenzenesulfonate

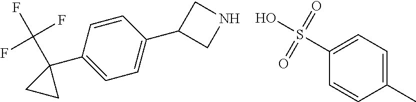 [SMILES: FC(F)(F)C1(c2ccc(C3CNC3)cc2)CC1 ; Cc1ccc(S(=O)(=O)O)cc1]

To a solution of tert-butyl 3-[4-[1-(trifluoromethyl)cyclopropyl]phenyl]azetidine-1-carboxylate (7.00 g, 20.5 mmol) in ethyl acetate (70 mL) was added p-toluenesulfonic acid (4.24 g, 24.6 mmol). The mixture was stirred at 80° C. for 3 h, cooled to room temperature, filtered and the filter cake was collected to give 4-methylbenzenesulfonic acid; 3-[4-[1-(trifluoromethyl)cyclopropyl]phenyl]azetidine (7600 mg, 18.4 mmol, 89.6% yield) as a white solid. MS (ESI): m/z=242.4 [M-TsOH+H]⁺

### Step a) tert-butyl 3-[4-[ ]-(trifluoromethyl)cyclopropyl]phenyl]azetidine-]-carboxylate

To a 500 mL vial equipped with a stir bar was added tert-butyl 3-bromoazetidine-1-carboxylate (CAS: tert-butyl 3-bromoazetidine-1-carboxylate) (8017 mg, 34.0 mmol), 1-bromo-4-(1-trifluoromethyl-cyclopropyl)-benzene (CAS: 1-bromo-4-(1-trifluoromethyl-cyclopropyl)-benzene) (9000 mg, 34.0 mmol), Ir[dF(CF₃)ppy]₂(dtbbpy)PF₆ (381 mg, 0.340 mmol), NiCl₂—glyme (37.3 mg, 0.170 mmol), 4-tert-butyl-2-(4-tert-butyl-2-pyridyl)pyridine (54.7 mg, 0.200 mmol), bis(trimethylsilyl)silyl-trimethyl-silane (8443 mg, 34.0 mmol) and Na₂CO₃ (7197 mg, 67.9 mmol) in DME (225 mL).The vial was sealed and placed under nitrogen. The reaction was stirred and irradiated with a 34 W blue LED lamp (7 cm away), with cooling fan to keep the reaction temperature at 25° C. for 20 h. LCMS showed the reaction was complete, the reaction was filtered and the filtrate was concentrated, the residue was purified by reverse phase flash chromatography (FA) and concentrated to give tert-butyl 3-[4-[1-(trifluoromethyl)cyclopropyl]phenyl]azetidine-1-carboxylate (7700 mg, 22.6 mmol, 66.4% yield) as a light yellow solid. MS: MS (ESI): m/z=286.0 [M-C₄H₈+H]⁺

### Example B.2

### 5-(azetidin-3-yl)-3-[[1-(trifluoromethyl)cyclopropyl]methyl]-1,2,4-oxadiazole; 4-methylbenzenesulfonic acid

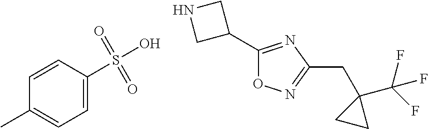 [SMILES: Cc1ccc(S(=O)(=O)O)cc1 ; FC(F)(F)C1(Cc2noc(C3CNC3)n2)CC1]

To a solution of tert-butyl 3-[3-[[1-(trifluoromethyl)cyclopropyl]methyl]-1,2,4-oxadiazol-5-yl]azetidine-1-carboxylate (320 mg, 0.920 mmol) in ethyl acetate (4 mL) was added p-toluenesulfonic acid (190 mg, 1.11 mmol), the mixture was stirred at 80° C. for 12 h. The mixture was cooled to 20° C. and evaporated under reduced pressure to give the residue. To the residue was then added 1 mL ethyl acetate, and a white solid was observed. the mixture was then filtered and washed with EA (5 mL) to give the cake 5-(azetidin-3-yl)-3-[[1-(trifluoromethyl)cyclopropyl]methyl]-1,2,4-oxadiazole; 4-methylbenzenesulfonic acid (335 mg, 0.800 mmol, 86.7% yield) as an off white solid. MS (ESI): m/z=248.2 [M-TsOH+H]⁺

### Step a) N′-hydroxy-2-[ ]-(trifluoromethyl)cyclopropyl]acetamidine

To a solution of hydroxylamine hydrochloride (839 mg, 12.1 mmol) and 2-[1-(trifluoromethyl)cyclopropyl]acetonitrile (CAS: 1454690-79-9) (900 mg, 6.04 mmol) in methanol (7 mL), water (7 mL), and sodium carbonate (1280 mg, 12.1 mmol) was added. The mixture was stirred at 50° C. for 12 h. The mixture was filtered, and the filtrate was concentrated under vacuum to remove the ethanol, then the residual solution was extracted with EtOAc (50 mL twice), the combined organic phase was dried over Na₂SO₄, concentrated to give N′-hydroxy-2-[1-(trifluoromethyl)cyclopropyl]acetamidine (700 mg, 3.84 mmol, 63.67% yield) as light yellow solid, which was used directly without further purification. MS (ESI): m/z=183.1 [M+H]⁺

### Step b) 03-[(Z)-[ ]-amino-2-[ ]-(trifluoromethyl)cyclopropyl]ethylidene]amino] 01-tert-butyl azetidine-1, 3-dicarboxylate

To a solution of DIPEA (1441 mg, 11.2 mmol), 0-(7-AZABENZOTRIAZOL-1-YL)-N,N,N′,N′-TETRAMETHYLURONIUM HEXAFLUOROPHOSPHATE (1696 mg, 4.46 mmol) and 1-BOC-azetidine-3-carboxylic acid (CAS: 142253-55-2) (928 mg, 4.61 mmol) in DCM (16 mL) was stirred for 5 min, then N′-hydroxy-2-[1-(trifluoromethyl)cyclopropyl]acetamidine (700 mg, 3.84 mmol) was added and stirred at 20° C. for 12 h. The reaction mixture was poured into H₂O (50 mL), extracted with EtOAc (50 mL×3), and purified with reversed phase column and lyophilized to give 03-[(Z)-[1-amino-2-[1-(trifluoromethyl)cyclopropyl]ethylidene]amino] 01-tert-butyl azetidine-1,3-dicarboxylate (1100 mg, 3.01 mmol, 78% yield) as light brown solid. MS (ESI): m/z=310.1 [M-C₄H₈+H]⁺

### Step c) tert-butyl 3-[3-[[ ]-(trifluoromethyl)cyclopropyl]methyl]-1,2,4-oxadiazol-5-yl]azetidine-1-carboxylate

A solution of 03-[(Z)-[1-amino-2-[1-(trifluoromethyl)cyclopropyl]ethylidene]amino] 01-tert-butyl azetidine-1,3-dicarboxylate (360 mg, 0.990 mmol) in DMF (18 mL) was stirred at 130° C. for 12 h. The reaction mixture was purified with reversed phase column (0.225% v/vFA) and lyophilized to give tert-butyl 3-[3-[[1-(trifluoromethyl)cyclopropyl]methyl]-1,2,4-oxadiazol-5-yl]azetidine-1-carboxylate (320 mg, 0.920 mmol, 93.5% yield) as a yellow oil. MS (ESI): m/z=292.1 [M-C₄H₈+H]⁺

### Example B.3

### 5-methyl-6-[[1-(trifluoromethyl)cyclopropyl]methoxy]pyridine-3-carboxylic acid

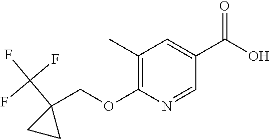 [SMILES: Cc1cc(C(=O)O)cnc1OCC1(C(F)(F)F)CC1]

To a solution of 6-chloro-5-methylnicotinic acid (CAS: 66909-29-3) (200 mg, 1.12 mmol) and 1-(trifluoromethyl)cyclopropanemethanol (CAS: 371917-17-8) (31.3 mg, 2.24 mmol) in DMSO (5 mL) was added Cs₂CO₃ (1.09 g, 3.36 mmol).The mixture was stirred at 145° C. for 16 h. The reaction mixture was concentrated by speedvac. The crude product was purified by re-crystallization from 0.33 N HCl in H₂O (3 mL) at 30° C. The title compound (95 mg, crude) was obtained as white solid, and was used directly without further purification. MS (ESI): m/z=276.1 [M+H]⁺

In analogy to Example B.3, the following building blocks were synthesized using the indicated heteroarene building block.

| | | | | MS, |
|---|---|---|---|---|
| | | | Building | ESI: |
| Ex. | Structure | Systematic Name | Blocks | m/z |
| B.4 | 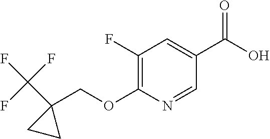 [SMILES: O=C(O)c1cnc(OCC2(C(F)(F)F)CC2)c(F)c1] | 5-fluoro-6-[[1- (trifluoromethyl) cyclopropyl]methoxy] pyridine-3-carboxylic acid | 6-chloro-5- fluoronicotinic acid (CAS: 38186-86-6) | 280.1 [M + H]⁺ |
| B.5 | 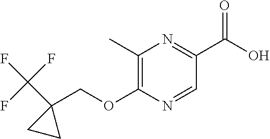 [SMILES: Cc1nc(C(=O)O)cnc1OCC1(C(F)(F)F)CC1] | 6-methyl-5-[[1- (trifluoromethyl) cyclopropyl]methoxy] pyrazine- 2-carboxylic acid | 5-chloro-6- methyl-2- pyrazine- carboxylic acid (CAS: 188781- 36-4) | 277.1 [M + H]⁺ |

### Example B.6

### 2-[[1-(trifluoromethyl)cyclopropyl]methoxy]pyrimidine-5-carboxylic acid

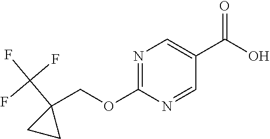 [SMILES: O=C(O)c1cnc(OCC2(C(F)(F)F)CC2)nc1]

To a solution of 2-chloropyrimidine-5-carboxylic acid (CAS:374068-01-6) (200 mg, 1.12 mmol) and 1-(trifluoromethyl)cyclopropanemethanol (31.3 mg, 2.24 mmol) in DMSO (5 mL) was added Cs₂CO₃ (1.09 g, 3.36 mmol).The mixture was stirred at 120° C. for 16 h. The reaction mixture was concentrated by speedvac. The crude product was purified by re-crystallization from 0.33 N HCl in H₂O (3 mL) at 30° C. The title compound (78 mg, crude) was obtained as white solid, which was used directly in the next step without further purification. MS (ESI): m/z=263.1 [M+H]⁺

### Example B.7

### 5-methyl-6-[[1-(trifluoromethyl)cyclopropyl]methoxy]pyridazine-3-carboxylic acid

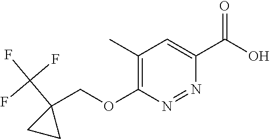 [SMILES: Cc1cc(C(=O)O)nnc1OCC1(C(F)(F)F)CC1]

To a solution of 6-chloro-4-methyl-3-[[1-(trifluoromethyl)cyclopropyl]methoxy]pyridazine (250 mg, 0.90 mmol) in DMF (5 mL) was added DIEA (2.7 mmol, 3.0 eq.), then Ac₂O (2.7 mmol, 3.0 eq.) Xantphos (0.135 mmol, 0.15 eq.), Pd(OAc)₂ (0.09 mmol, 0.1 eq.), oxalic acid dihydrate (2.7 mmol, 3.0 eq.) was added to the mixture under N₂ atmosphere.The mixture was stirred at 100° C. for 16 h. The crude product was purified by re-crystallization from 0.33 N HCl in H₂O (3 mL) at 30° C., diluted with H₂O (2 mL) and extracted with AcOEt (5 mL×3). The combined organic layers were dried over Na₂SO₄, filtered and concentrated under reduced pressure to give a residue. The title compound (235 mg, crude) was obtained as light yellow oil. MS (ESI): m/z=277.2 [M+H]⁺

### Step a) 6-chloro-4-methyl-3-[[ ]-(trifluoromethyl)cyclopropyl]methoxy]pyridazine

To a solution of 6-chloro-4-methylpyridazin-3-ol (CAS: 1834-27-1) (500 mg, 3.45 mmol) and 1-(trifluoromethyl)cyclopropanemethanol (966 mg, 6.90 mmol, 2.0 eq.) in THF (5 mL) was added PS-TPP (2.0 eq.),then DIAD (1.5 eq.) was added to the mixture under N₂ atmosphere. The mixture was stirred at 30° C. for 16 h. The residue was purified by TLC. The title compound (250 mg, 92.0% purity) was obtained as light yellow oil. MS (ESI): m/z=267.1 [M+H]⁺

The synthesis of the following Building blocks has been described in the literature:

| Ex. | Structure | Systematic Name | Reference |
|---|---|---|---|
| B.8 | 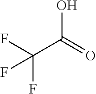 | 6-(2-methoxy-4- (trifluoromethyl) benzyl)-2- azaspiro[3.3]heptane | WO 2020/104494 A1 |
| | 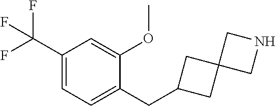 [SMILES: COc1cc(C(F)(F)F)ccc1CC1CC2(CNC2)C1] | | |
| B.11 | 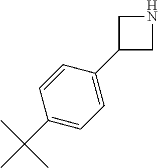 | 3-(4-(tert- butyl)phenyl)azetidine 2,2,2-trifluoroacetate | WO2019/ 180185 A1 |
| | 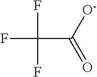 | | |

### Example B.9

### 6-(2,4-difluorobenzyl)-2-azaspiro[3.3]heptane 4-methylbenzenesulfonate

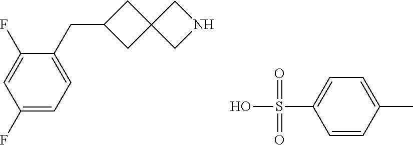 [SMILES: Fc1ccc(CC2CC3(CNC3)C2)c(F)c1 ; Cc1ccc(S(=O)(=O)O)cc1]

To a solution of tert-butyl 6-(2,4-difluorobenzyl)-2-azaspiro[3.3]heptane-2-carboxylate (135 mg, 417 μmol) in ethyl acetate (3 mL) was added 4-methylbenzenesulfonic acid hydrate (79.4 mg, 417 μmol) and the reaction mixture was stirred at 80° C. for 18 h. Volatiles were removed in vacuo to yield 166 mg of the crude desired product which was used without further purification. MS (ESI): m/z=224.2 [M+H]⁺

### Step a) tert-butyl 6-(2,4-difluorobenzyl)-2-azaspiro[3.3]heptane-2-carboxylate

To a solution of tert-butyl 6-formyl-2-azaspiro[3.3]heptane-2-carboxylate (CAS: 1440960-67-7) (500 mg, 2.22 mmol) in dry dioxane (8 mL) under an inert atmosphere was added 4-methylbenzenesulfonohydrazide (413 mg, 2.22 mmol) and the reaction mixture was stirred at 80° C. for 2 h. LCMS revealed formation of tosylhydrazone intermediate. The reaction was then cooled down to r.t followed by addition of (2,4-difluorophenyl)boronic acid (526 mg, 3.33 mmol) and K₂CO₃ (460 mg, 3.33 mmol) and the reaction was then stirred at 110° C. for 18 h. The reaction mixture was then diluted with dichloromethane and extracted with aq. NaHCO₃(1 M solution), the organic phase was collected and the aqueous phase was back-extracted with dichloromethane. The combined organic phases were dried over sodium sulfate and evaporated down to dryness. The crude material was purified by flash chromatography eluting with a mixture of heptane and ethyl acetate (5% to 50%) to yield the title compound (137 mg, 18.7%). MS (ESI): m/z=268.2 [M−tBu+H]⁺

### Example B.10

### 6-(4-(trifluoromethyl)phenoxy)-2-azaspiro[3.3]heptane bis(4-methylbenzenesulfonate)

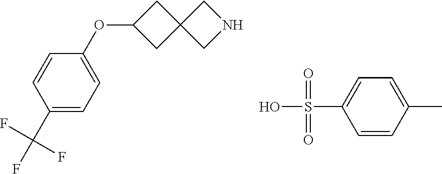 [SMILES: FC(F)(F)c1ccc(OC2CC3(CNC3)C2)cc1 ; Cc1ccc(S(=O)(=O)O)cc1]

To a solution of tert-butyl 6-(4-(trifluoromethyl)phenoxy)-2-azaspiro[3.3]heptane-2-carboxylate (167 mg, 467 μmol) in ethyl acetate (1.11 mL) was added 4-methylbenzenesulfonic acid monohydrate (93.3 mg, 491 μmol). Then, the reaction mixture was refluxed for 16 h. The reaction mixture was cooled and the solvent was evaporated to give the title compound, 6-(4-(trifluoromethyl)phenoxy)-2-azaspiro[3.3]heptane 4-methylbenzenesulfonate (212 mg, 444 μmol, 95.1% yield), which was obtained as a white solid. MS (ESI): m/z=258.2 [M+H]⁺

### Step a) tert-butyl 6-(4-(trifluoromethyl)phenoxy)-2-azaspiro[3.3]heptane-2-carboxylate

To a solution of 4-(trifluoromethyl)phenol (0.5 g, 3.08 mmol) in dry DMF (12 mL) under an inert atmosphere was added NaH (123 mg, 3.08 mmol) and the reaction mixture was stirred at r.t. for 10 min followed by addition of tert-butyl 6-((methylsulfonyl)oxy)-2-azaspiro[3.3]heptane-2-carboxylate (CAS: 1239320-11-6) (899 mg, 3.08 mmol). The reaction mixture was then stirred at 90° C. for 18 h. The reaction mixture was stirred at 90° C. for another 20 h. Reaction was cooled down to r.t., followed by addition of 4-(trifluoromethyl)phenol (250 mg, 1.54 mmol) and NaH (61.7 mg, 1.54 mmol), the reaction was then stirred at r.t. for 10 min and at 90° C. for another 24 h. Volatiles were removed in vacuo and the crude residue was partitioned between ethyl acetate and aq. Na₂CO₃ 1 M solution, the organic phase was collected and the aqueous phase was back-extracted with ethyl acetate. The combined organic phases were dried over sodium sulfate and evaporated down to dryness. The crude material was purified by flash chromatography, eluting with a mixture of heptane and ethyl acetate (5% to 25%) to yield the title compound (840 mg). MS (ESI): m/z=302.2 [M−tBu+H]⁺

In analogy to Example B.10, the following building blocks were synthesized using the indicated phenol building block.

| | | | | MS, |
|---|---|---|---|---|
| | | | Building | ESI: |
| Ex. | Structure | Systematic Name | Blocks | m/z |
| B.12 | 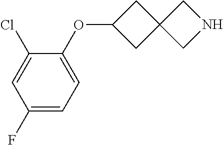 [SMILES: Fc1ccc(OC2CC3(CNC3)C2)c(Cl)c1] | 6-(2-chloro-4- fluorophenoxy)-2- azaspiro[3.3]heptane 4- methylbenzenesulfonate | 2-chloro-4- fluorophenol | 242.2 [M + H]⁺ |
| | 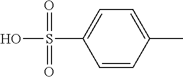 | | | |
| B.13 | 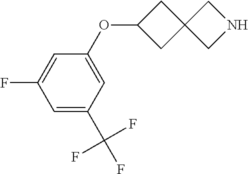 [SMILES: Fc1cc(OC2CC3(CNC3)C2)cc(C(F)(F)F)c1] | 6-[3-fluoro-5- (trifluoromethyl) phenoxy]-2- azaspiro[3.3]heptane 4- methylbenzenesulfonate | 3-fluoro-5- (trifluoromethyl) phenol (CAS: 172333-87-8) | 276.2 [M + H]⁺ |
| | 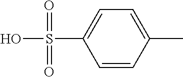 | | | |
| B.16 | 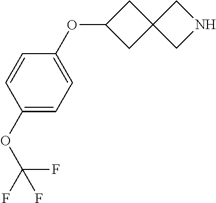 [SMILES: FC(F)(F)Oc1ccc(OC2CC3(CNC3)C2)cc1] | 6-[4- (trifluoromethoxy) phenoxy]-2- azaspiro[3.3]heptane 4- methylbenzenesulfonate | 4- (trifluoromethoxy) phenol | 274.2 [M + H]⁺ |
| | 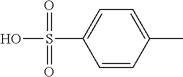 | | | |
| B.17 | 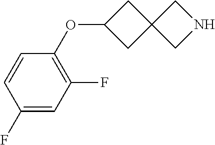 [SMILES: Fc1ccc(OC2CC3(CNC3)C2)c(F)c1] | 6-(2,4- difluorophenoxy)-2- azaspiro[3.3]heptane 4- methylbenzenesulfonate | 2,4- difluorophenol | 226.2 [M + H]⁺ |
| | 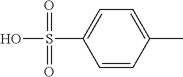 | | | |
| B.27 | 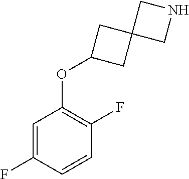 [SMILES: Fc1ccc(F)c(OC2CC3(CNC3)C2)c1] | 6-(2,5- difluorophenoxy)-2- azaspiro[3.3]heptane 4- methylbenzenesulfonate | 2,5- difluorophenol | 226.2 [M + H]⁺ |
| | 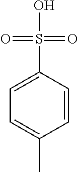 | | | |
| B.28 | 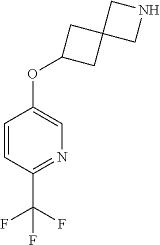 [SMILES: FC(F)(F)c1ccc(OC2CC3(CNC3)C2)cn1] | 6-((6- (trifluoromethyl) pyridin-3-yl)oxy)-2- azaspiro[3.3]heptane 4- methylbenzenesulfonate | 6- (trifluoromethyl) pyridin-3-ol (CAS: 216766- 12-0) | 259.2 [M − C₇H₈O₃S + H]⁺ |
| | 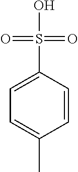 | | | |
| B.29 | 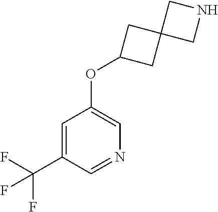 [SMILES: FC(F)(F)c1cncc(OC2CC3(CNC3)C2)c1] | 6-((5- (trifluoromethyl) pyridin-3-yl)oxy)-2- azaspiro[3.3]heptane 4- methylbenzenesulfonate | 5- (trifluoromethyl) pyridin-3-ol (CAS: 186593- 14-6) | 259.2 [M − C₂HF₃O₂ + H]⁺ |
| | 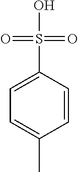 | | | |
| B.30 | 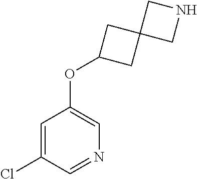 [SMILES: Clc1cncc(OC2CC3(CNC3)C2)c1] | 6-((5-chloropyridin-3- yl)oxy)-2- azaspiro[3.3]heptane 4- methylbenzenesulfonate | 5- chloropyridin- 3-ol (CAS: 74115-12-1) | 225.1 [M − C₇H₈O₃S + H]⁺ |
| | 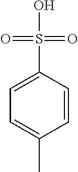 | | | |
| B.31 | 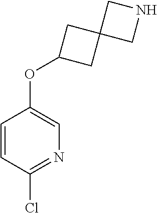 [SMILES: Clc1ccc(OC2CC3(CNC3)C2)cn1] | 6-((6-chloropyridin-3- yl)oxy)-2- azaspiro[3.3]heptane 4- methylbenzenesulfonate | 6- chloropyridin- 3-ol (CAS: 41288-96-4) | 225.1 [M − C₇H₈O₃S + H]⁺ |
| | 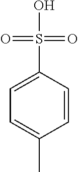 | | | |

### Example B.14

### 6-[(3,4-difluorophenyl)methyl]-2-azaspiro13.31heptane;4-methylbenzenesulfonic acid

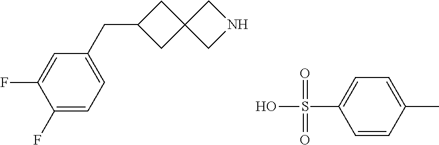 [SMILES: Fc1ccc(CC2CC3(CNC3)C2)cc1F ; Cc1ccc(S(=O)(=O)O)cc1]

To an solution of tert-butyl 6-(3,4-difluorobenzyl)-2-azaspiro[13.3]heptane-2-carboxylate (140 mg, 433 μmol) in ethyl acetate (35.6 mL) was added 4-methylbenzenesulfonic acid monohydrate (82.4 mg, 433 μmol) and the mixture was heated at reflux for 1 h. The clear, colorless solution was allowed to cool down to RT and concentrated. The residue was dissolved in DCM, ether was added, and was stood at 4° C. overnight to crystallize. The crystals were isolated and washed with Et₂O and dried under high vacuum to yield the title compound (110 mg, 51% yield) as a light brown semi-solid. MS (ESI): m/z=224.1 [M+H]⁺

### Step a) (3,4-difluorobenzyl)triphenylphosphonium bromide

Under argon, triphenylphosphine (3.8 g, 14.5 mmol) was dissolved in acetonitrile (50 mL) and 4-(bromomethyl)-1,2-difluorobenzene (CAS: 85118-01-0) (3.00 g, 1.86 mL, 14.5 mmol) was added. The mixture was stirred at 80° C. for 3 h. The suspension was allowed to cool to RT. Ether was added and the mixture was stirred 30 min at r.t.. The solid was filtrated and dried under high vacuum to yield the title compound (7.0 g, crude), which was used directly in the next step without further purification. MS (ESI): m/z=389.2 [M+H]⁺

### Step b) tert-butyl 6-(3,4-difluorobenzylidene)-2-azaspiro[3.3]heptane-2-carboxylate

Under argon at −78° C., (3,4-difluorobenzyl)triphenylphosphonium bromide (4.00 g, 8.52 mmol) was dissolved in dry THF (50 mL) and lithium bis(trimethylsilyl)amide solution (17 mL of 1 M solution) was added. The reaction mixture was stirred at −78° C. for 2 h. Then at RT, tert-butyl 6-oxo-2-azaspiro[3.3]heptane-2-carboxylate (CAS: 1181816-12-5) (3.6 g, 17 mmol) was added and the mixture was stirred at 85° C. overnight. The crude material was evaporated and dried. The residue was purified by flash chromatography (0% to 80% EtOAc in heptane) to yield the title compound (210 mg, 7% yield) as an amorphous, colourless solid. MS (ESI): m/z=266.2 [M+H]⁺

### Step c) tert-butyl 6-(3,4-difluorobenzyl)-2-azaspiro[3.3]heptane-2-carboxylate

Tert-butyl 6-(3,4-difluorobenzylidene)-2-azaspiro[3.3]heptane-2-carboxylate (0.142 g, 442 μmol) was dissolved in ethyl acetate (3 mL). The flask was purged and backfilled with argon (×3). Pd—C(47 mg, 44.2 μmol) was added and the reaction was stirred under H₂ for 2 h. The reaction mixture was filtered through a celite pad, washed with EtOAc and dried under vacuum. The crude residue was used directly in the next step without further purification.

### Example B.15

### 3-[3-chloro-4-(trifluoromethoxy)phenyl]azetidine 4-methylbenzenesulfonate

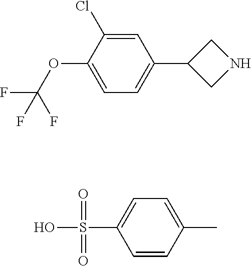 [SMILES: FC(F)(F)Oc1ccc(C2CNC2)cc1Cl ; Cc1ccc(S(=O)(=O)O)cc1]

A solution of tert-butyl 3-(3-chloro-4-(trifluoromethoxy)phenyl)azetidine-1-carboxylate (75 mg, 213 μmol) and 4-methylbenzenesulfonic acid hydrate (48.7 mg, 256 μmol) in EtOAc (0.6 mL) was stirred at reflux for 1 h. A suspension was formed which was cooled in the fridge, then filtered and washed with ethyl acetate to get the title compound as a colorless solid (0.080 g; 88.5%). MS (ESI): m/z=252.1 [M+H]⁺.

### Step a) tert-butyl 3-(3-chloro-4-(trifluoromethoxy)phenyl)azetidine-1-carboxylate

To a solution of 4-bromo-2-chloro-1-(trifluoromethoxy)benzene (CAS: 158579-80-7) (500 mg, 1.82 mmol) in dry DME (14 mL) under an inert atmosphere was added tert-butyl 3-bromoazetidine-1-carboxylate (643 mg, 2.72 mmol), tris(trimethylsilyl)silane (451 mg, 560 μL, 1.82 mmol), (Ir[dF(CF₃)ppy]₂(dtbpy))PF₆ (20.4 mg, 18.2 μmol) and sodium carbonate (385 mg, 3.63 mmol). In a separate vial was added nickel(II) chloride ethylene glycol dimethyl ether complex (20 mg) and 4,4′-di-tert-butyl-2,2′-dipyridyl (25 mg), the vial was sealed and purged with argon followed by addition of dry DME (2 mL). The catalyst solution was sonicated for 5 min after which 0.2 mL of the solution was syringed into the previous reaction mixture. The reaction mixture was degased by bubbling argon into the solution for 10 min while stirring. The reaction was then stirred at r.t. under blue LED irradiation for 18 h. The reaction mixture was diluted with ethyl acetate and extracted with aq. Na₂CO₃ 1 M solution, the organic phase was collected and the aqueous phase was back-extracted with ethyl acetate. The combined organic phases were dried over sodium sulfate and evaporated down to dryness. The crude material was purified by flash chromatography, eluting with a mixture of heptane and ethyl acetate (5% to 25%) to yield the title compound (531 mg) which was further purified by SFC to yield the title compound (364 mg). MS (ESI): m/z=296.1 [M+H]⁺

### Example B.18

### 6-(3,4-difluorophenoxy)-2-azaspiro[3.3]heptane; 4-methylbenzenesulfonic acid

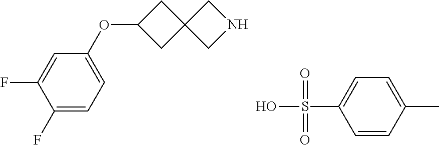 [SMILES: Fc1ccc(OC2CC3(CNC3)C2)cc1F ; Cc1ccc(S(=O)(=O)O)cc1]

To an solution of tert-butyl 6-(3,4-difluorophenoxy)-2-azaspiro[3.3]heptane-2-carboxylate (2.6 g, 7.99 mmol) in ethyl acetate (35.6 mL) was added 4-methylbenzenesulfonic acid monohydrate (1.52 g, 7.99 mmol) and the mixture was heated at reflux for 1 h. The solution was allowed to cool down to RT, then concentrated. The residue was dissolved in DCM, ether was added, and was stood at 4° C. overnight to crystallize. The crystals were isolated, washed with Et₂O, and dried under high vaccum to yield the title compound (2.5 g, 54.3%) as a light brown solid. MS (ESI): m/z=226.1 [M+H]⁺.

### Step a) tert-butyl 6-(3,4-difluorophenoxy)-2-azaspiro[3.3]heptane-2-carboxylate

To a solution of 3,4-difluorophenol (1.34 g, 10.3 mmol), tert-butyl 6-hydroxy-2-azaspiro[3.3]heptane-2-carboxylate (2.00 g, 9.38 mmol) and triphenylphosphine (2.95 g, 11.3 mmol) in THF (46.9 mL) was added DIAD (2.28 g, 2.19 ml, 11.3 mmol) dropwise at 0° C. and the reaction was stirred at r.t. for 18 h. Triphenylphosphine (1.48 g, 5.63 mmol), followed by DIAD (1.14 g, 1.09 ml, 5.63 mmol) were added and the reaction was stirred at r.t. for 6 h. The reaction mixture was poured into sat. aq. NaHCO₃ solution (50 mL) and EtOAc (30 mL) was added. The phases were separated and the aq. phase was extracted with EtOAc. The combined organic layers were washed with brine, dried over sodium sulfate, filtered and concentrated to give an orange oil. The crude product was immobilized on Isolute and purified by column chromatography (0-30% EtOAc in heptane) to afford the title compound (2.6 g, 7.99 mmol, 85.2% yield) as a yellow solid.

### Example B.19

### 3-((4-(trifluoromethoxy)benzyl)oxy)azetidine 4-methylbenzenesulfonate

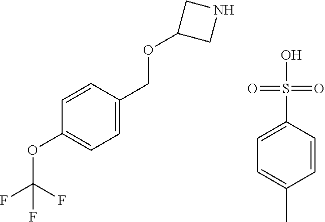 [SMILES: FC(F)(F)Oc1ccc(COC2CNC2)cc1 ; Cc1ccc(S(=O)(=O)O)cc1]

To a solution of tert-butyl 3-((4-(trifluoromethoxy)benzyl)oxy)azetidine-1-carboxylate (1.00 g, 2.88 mmol) in EtOAc (6.85 mL) was added 4-methylbenzenesulfonic acid monohydrate (575 mg, 3.02 mmol). The reaction mixture was refluxed for 18 h. The reaction mixture was cooled and the solvent was evaporated to yield the title compound (1.24 g, 67.1%). MS (ESI): m/z=248.2 [M-C₇H₈O₃S+H]⁺

### Step a) tert-butyl 3-((4-(trifluoromethoxy)benzyl)oxy)azetidine-]-carboxylate

To a solution of tert-butyl 3-hydroxyazetidine-1-carboxylate (1.00 g, 5.77 mmol) in dry THF (25.0 mL) potassium tert-butoxide (1.65 M solution in THF) (3.85 mL, 6.35 mmol) was added. The reaction mixture was stirred at room temperature for 30 min, followed by addition of 1-(bromomethyl)-4-(trifluoromethoxy)benzene (1.47 g, 5.77 mmol). The reaction mixture was stirred at room temperature for 20 h, diluted with EtOAc and washed with 1 M aq. NaHCO₃ solution. The organic phase was collected and the aqueous phase underwent back-extraction with EtOAc. The combined organic layers were dried over Na₂SO₄ and evaporated to yield the title compound (978 mg, 48.3%). MS (ESI): m/z=292.2 [M-C₄H₉+H]⁺

In analogy to Example B.19, the examples in the following table were synthesized, using the respective commercially available benzyl bromides. In some cases, trifluoroacetate salts were made in place of the methylbenzenesulfonate salts, by substituting the 4-methylbenzenesulfonic acid monohydrate with trifluoroacetic acid (7 eq.). In some cases the free base was isolated following an aqueous NaHCO₃ workup and extraction with CH₂Cl₂. In some examples a substituted tert-butyl 3-hydroxyazetidine-1-carboxylate building block was used.

| | | | | | MS, |
|---|---|---|---|---|---|
| | | | | | ESI: |
| Ex. | Structure | | Systematic Name | Building Blocks | m/z |
| B.20 | 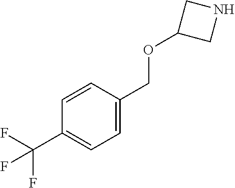 [SMILES: FC(F)(F)c1ccc(COC2CNC2)cc1] | 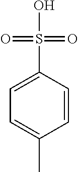 | 3-((4- (trifluoromethyl)ben- zyl)oxy)azetidine 4- methylbenzenesulfo- nate | 1-(bromomethyl)- 4- (trifluoromethyl)ben- zene (CAS: 402-49-3) | 232.2 [M − C₇H₈O₃S + H]⁺ |
| B.21 | 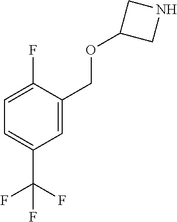 [SMILES: Fc1ccc(C(F)(F)F)cc1COC1CNC1] | 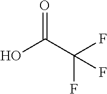 | 3-((2-fluoro-5- (trifluoromethyl)ben- zyl)oxy)azetidine 2,2,2-trifluoroacetate | 2-(bromomethyl)- 1-fluoro-4- (trifluoromethyl)ben- zene (CAS: 220239-69-0) | 250.2 [M − C₂HF₃O₂ + H]⁺ |
| B.22 | 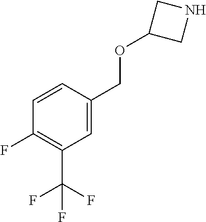 [SMILES: Fc1ccc(COC2CNC2)cc1C(F)(F)F] | 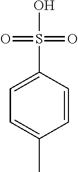 | 3-((3-fluoro-4- (trifluoromethyl)ben- zyl)oxy)azetidine 4- methylbenzenesulfo- nate | 4-(bromomethyl)- 2-fluoro-1- (trifluoromethyl)ben- zene (CAS: 184970-26-1) | 250.2 [M − C₇H₈O₃S + H]⁺ |
| B.23 | 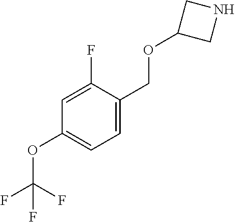 [SMILES: Fc1cc(OC(F)(F)F)ccc1COC1CNC1] | 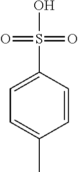 | 3-((2-fluoro-4- (trifluoromethoxy)ben- zyl)oxy)azetidine 4- methylbenzenesulfo- nate | 1-(bromomethyl)- 2-fluoro-4- (trifluoromethoxy) benzene (1240257-47-9) | 266.2 [M − C₇H₈O₃S + H]⁺ |
| B.24 | 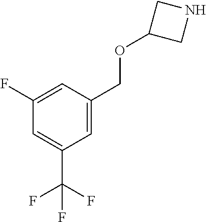 [SMILES: Fc1cc(COC2CNC2)cc(C(F)(F)F)c1] | 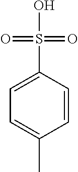 | 3-((3-fluoro-5- (trifluoromethyl)ben- zyl)oxy)azetidine 4- methylbenzenesulfo- nate | 1-(bromomethyl)- 3-fluoro-5- (trifluoromethyl)ben- zene (CAS: 239087-09-3) | 250.2 [M − C₇H₈O₃S + H]⁺ |
| B.25 | 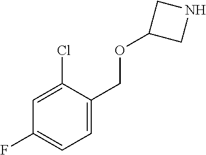 [SMILES: Fc1ccc(COC2CNC2)c(Cl)c1] | 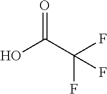 | 3-((2-chloro-5- fluorobenzyl)oxy)aze- tidine 2,2,2- trifluoroacetate | 1-(bromomethyl)- 2-chloro-4- fluorobenzene (CAS: 45767-66- 6) | 216.1 [M − C₂HF₃O₂ + H]⁺ |
| B.26 | 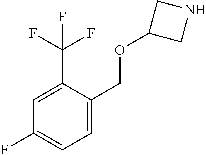 [SMILES: Fc1ccc(COC2CNC2)c(C(F)(F)F)c1] | 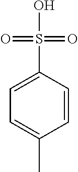 | 3-((4-fluoro-2- (trifluoromethyl)ben- zyl)oxy)azetidine 4- methylbenzenesulfo- nate | 1-(bromomethyl)- 4-fluoro-2- (trifluoromethyl)ben- zene (CAS: 206860-48-2) | 250.2 [M − C₇H₈O₃S + H]⁺ |
| B.43 | 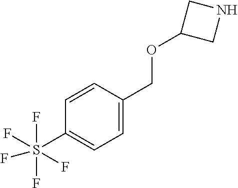 [SMILES: FS(F)(F)(F)(F)c1ccc(COC2CNC2)cc1] | 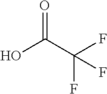 | [4-(azetidin-3- yloxymethyl)phenyl]- pentafluoro-λ⁶- sulfane; 2,2,2- trifluoroacetic acid | 4- (pentafluorosulfur) benzyl bromide (CAS: 1126969- 29-6) | 290.1 [M − C₂HF₃O₂ + H]⁺ |
| B.44 | 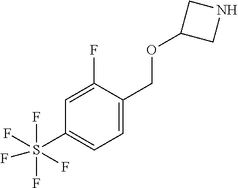 [SMILES: Fc1cc(S(F)(F)(F)(F)F)ccc1COC1CNC1] | 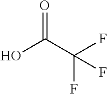 | [4-(azetidin-3- yloxymethyl)-3- fluoro-phenyl]- pentafluoro-λ⁶- sulfane; 2,2,2- trifluoroacetic acid | 2-fluoro-4- (pentafluorosulfur) benzyl bromide (CAS: 1240257- 17-3) | 308.2 [M − C₂HF₃O₂ + H]⁺ |
| B.45 | 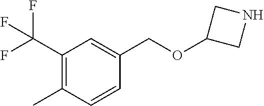 [SMILES: Cc1ccc(COC2CNC2)cc1C(F)(F)F] | | 3-[[4-methyl-3- (trifluoromethyl)phe- nyl]methoxy]azetidine | 4-methyl-3- (trifluoromethyl)ben- zyl bromide (CAS: 261952- 19-6) | 246.2 [M + H]⁺ |
| B.47 | 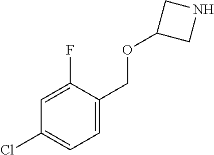 [SMILES: Fc1cc(Cl)ccc1COC1CNC1] | 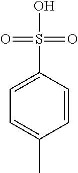 | 3-[(4-chloro-2- fluoro- phenyl)methoxy]azeti- dine; 4- methylbenzenesulfo- nic acid | 4-chloro-2- fluorobenzyl bromide (CAS: 71916-82-0) | 216.1 [M − C₇H₈O₃S + H]⁺ |
| B.48 | 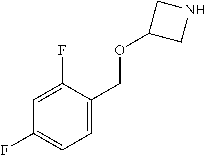 [SMILES: Fc1ccc(COC2CNC2)c(F)c1] | 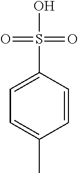 | 3-[(2,4- difluorophenyl)meth- oxy]azetidine; 4- methylbenzenesulfro- nic acid | 2,4- difluorobenzyl bromide (CAS: 23915-07-3) | 216.1 [M − C₇H₈O₃S + H]⁺ |
| B.50 | 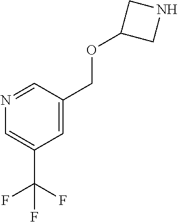 [SMILES: FC(F)(F)c1cncc(COC2CNC2)c1] | 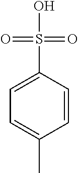 | 3-(azetidin-3- yloxymethyl)-5- (trifluoromethyl)pyri- dine; 4- methylbenzenesulfo- nic acid | 3-bromomethyl- 5- (trifluoromethyl)py- ridine (CAS: 1227574-31-3) | 233.4 [M − C₇H₈O₃S + H]⁺ |
| B.52 | 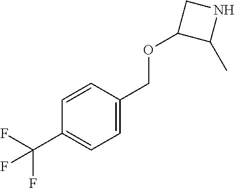 [SMILES: CC1NCC1OCc1ccc(C(F)(F)F)cc1] | 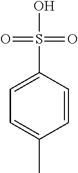 | 2-methyl-3-[[4- (trifluoromethyl)phe- nyl]methoxy]azetidine; 4- methylbenzenesulfo- nic acid | 1-(bromomethyl)- 4- (trifluoromethyl)ben- zene and tert- butyl 3-hydroxy- 2- methylazetidine- 1-carboxylate (CAS: 1354955- 59-1) | 246.2 [M − C₇H₈O₃S + H]⁺ |

### Example B.32

### 3-bromo-4-[5-(2,2-dimethylpropyl)-1,2,4-oxadiazol-3-yl]benzoic acid

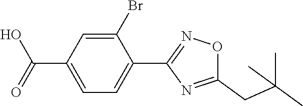 [SMILES: CC(C)(C)Cc1nc(-c2ccc(C(=O)O)cc2Br)no1]

To a solution of methyl 3-bromo-4-[5-(2,2-dimethylpropyl)-1,2,4-oxadiazol-3-yl]benzoate (15 mg, 0.042 mmol) in THF (1.5 ml), methanol (1.2 ml) and water (0.3 ml) was added LiOH, H₂O (9 mg, 0.21 mmol) at −5° C. and stirred the reaction mixture at 0° C. for 2 h. The reaction mixture was acidified with 0.5 N aq.HCl. The volatiles were removed in vacuo to give the title compound (25 mg, crude mass). MS: m/z=339.0 [M−H]

### Step a) methyl 3-bromo-4-[(1E)-(hydroxyimino)methyl]benzoate

Methyl 3-bromo-4-formylbenzoate (300 mg, 1.23 mmol) was taken up in ethanol (6 mL) followed by the addition of triethylamine (0.34 mL, 2.47 mmol), hydroxylammonium chloride (129 mg, 1.85 mmol) and water (3 mL) and the mixture was heated at 70° C. for 1 h. Ethanol was removed in vacuo and the white solid filtered through sintered glass, washed with water and dried in vacuo to give the title compound (270 mg, 85%) which was carried to the next step without further purification.

### Step b) methyl 3-bromo-4-[(Z)—N′-hydroxycarbamimidoyl]benzoate

A solution of N-chlorosuccinimide (141 mg, 1.06 mmol) in DMF (1.5 mL) was added slowly to a solution of methyl 3-bromo-4-[(1E)-(hydroxyimino)methyl]benzoate (260 mg, 1.01 mmol) in DMF (3.5 mL) at 50° C. After complete addition, the reaction mixture was allowed to stir for 30 min. at the same temperature. The reaction mixture was then cooled to 5° C. and ammonium hydroxide (0.1 mL) was added dropwise. During addition the temperature was maintained between 0-10° C. The reaction mixture was allowed to stir for 15 min. at the same temperature. Ethyl acetate was added to the cooled reaction mixture, followed by brine solution and the aqueous layer was extracted with ethyl acetate. The combined organic part was dried, filtered and evaporated under reduced pressure to give the title compound (325 mg, crude) as a brown liquid. MS (ESI): m/z=273.1 [M+H]⁺

### Step c) methyl 3-bromo-4-[5-(2,2-dimethylpropyl)-1, 2,4-oxadiazol-3-yl]benzoate

To the solution of 3,3-dimethylbutanoic acid (136 mg, 1.17 mmol) in DMF (5 mL) at 25° C. was added CDI (228 mg, 1.41 mmol) and the reaction mixture was stirred at 50° C. for 45 min. Then methyl 3-bromo-4-[(Z)—N′-hydroxycarbamimidoyl]benzoate (320 mg, 1.17 mmol) was added and reaction mixture was heated at 100° C. for 1 h. Reaction mixture was cooled to 25° C.; water was added and the mixture was extracted with EtOAc. The organic layer was washed with brine, dried, filtered and concentrated under reduced pressure to dryness. The residue was purified by silica gel chromatography with an eluent mixture of ethyl acetate and hexane (2%-5%) providing the title compound (25 mg, 6%) as a colorless gum.

### Example B.33

### rac-(3aR,6aS)-5-[2-fluoro-4-(trifluoromethyl)phenoxy]-1,2,3,3a,4,5,6,6a-octahydrocyclopenta[c]pyrrole 2,2,2-trifluoroacetate

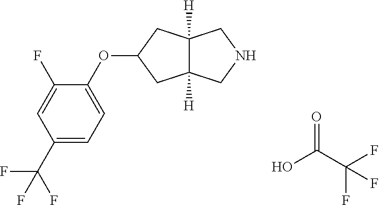 [SMILES: Fc1cc(C(F)(F)F)ccc1OC1C[C@H]2CNC[C@H]2C1 ; O=C(O)C(F)(F)F]

To a solution of tert-butyl 5-(2-fluoro-4-(trifluoromethyl)phenoxy)hexahydrocyclopenta[c]pyrrole-2(1H)-carboxylate (40 g, 103 mmol) in DCM (835 mL) was added 2,2,2-trifluoroacetic acid (117 g, 1.03 mol). The mixture was stirred for 4 h at room temperature. The solvent was removed under reduced pressure, TFA was coevaporated with toluene. The crude was used in the next step without further purification.

### Step a) rac-tert-butyl (3aR,6aS)-5-(2-fluoro-4-(trifluoromethyl)phenoxy)hexahydrocyclopenta[c]pyrrole-2(]H)-carboxylate

To a solution of 2-fluoro-4-(trifluoromethyl)phenol (67.5 mg, 50 μL, 375 μmol) in THF (1.87 mL) was added tert-butyl 5-hydroxyhexahydrocyclopenta[c]pyrrole-2(1H)-carboxylate (85.2 mg, 375 μmol) followed by triphenylphosphine (108 mg, 412 μmol). The mixture was cooled to 0° C. and diisopropyl (E)-diazene-1,2-dicarboxylate (83.4 mg, 412 μmol) was added. The mixture was allowed to warm up to room temperature and stirred for 24 h. The reaction was stopped by addition of sat. aq. Na₂CO₃ (10 mL). The aqueous phase was extracted with DCM (3×20 mL). The organic phases were washed with NaOH solution (30 mL, 1 M aqueous solution) and brine. The organic phases were dried over MgSO₄ and the solvent evaporated under reduced pressure. The crude was purified by column chromatography using heptane/ethyl acetate (0-30% EA) as solvent. The title compound (93 mg, 234 μmol, 63% yield) was obtained as a white solid. MS (ESI): m/z=334.2 [M−tBu+H]⁺

In analogy to Example B.33, examples in the following table were synthesized, using the respective commercially available phenols. In some cases, methylbenzenesulfonate salts were made in place of the trifluoroacetate salts, by trifluoroacetic acid with 4-methylbenzenesulfonic acid.

| | | | | | MS, |
|---|---|---|---|---|---|
| | | | | | ESI: |
| Ex. | Structure | | Systematic Name | Building Blocks | m/z |
| B.34 | 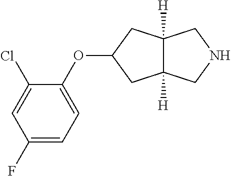 [SMILES: Fc1ccc(OC2C[C@H]3CNC[C@H]3C2)c(Cl)c1] | 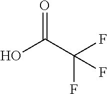 | rac-(3aS,6aR)-5-(2- chloro-4-fluoro- phenoxy)- 1,2,3,3a,4,5,6,6a- octahydrocyclopenta [c]pyrrole 2,2,2- trifluoroacetate | 2-chloro-4- fluorophenol | 256.2 [M + H]⁺ |

### Example B.35

### 3-[2-[4-(difluoromethoxy)phenyl]ethynyl]azetidine; hydrochloride salt

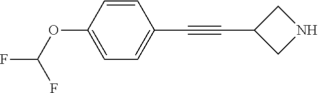 [SMILES: FC(F)Oc1ccc(C#CC2CNC2)cc1]

Tert-butyl 3-((4-(trifluoromethoxy)phenyl)ethynyl)azetidine-1-carboxylate (56.6 mg, 166 μmol) was dissolved in dioxane (0.5 mL). The solution was cooled down with an icebath. 4 M HCl in dioxane (415 μL, 1.66 mmol) was added. The reaction stirred at room temperature for 6 h. The crude reaction mixture was evaporated to dryness. The residue was triturated with diisopropylether. The solvent was filtered and the solid residue was dried under high vacuum. The title compound (30 mg, 76% yield) was isolated as a white powder. MS (ESI): m/z=242.2 [M+H]⁺.

### Step a) Tert-butyl 3-((4-(trifluoromethoxy)phenyl)ethynyl)azetidine-1-carboxylate

Tert-butyl 3-ethynylazetidine-1-carboxylate (100 mg, 552 μmol), 1-bromo-4-(trifluoromethoxy)benzene (199 mg, 123 μL, 828 μmol), bis(triphenylphosphine)palladium (II) chloride (31 mg, 44.1 μmol), copper (I) iodide (2.1 mg, 11 μmol) and TEA (558 mg, 769 μL, 5.52 mmol) were dissolved in THF (1.5 mL) under argon. The mixture was heated at 70° C. for 30 h. The mixture was diluted with EtOAc, filtered through dicalite and evaporated. The residue was purified by silica gel flash chromatography eluting with 0-10% EtOAc/Heptane gradient to yield the title compound (56.6 mg, 30%) as a yellow oil. [M−tBu+H]⁺=286.2.

In analogy to Example B.35, examples in the following table were synthesized, using the respective commercially available aryl halides. In some cases the free base was isolated following an aqueous NaHCO₃workup and extraction with CH₂Cl₂.

| | | | | MS, |
|---|---|---|---|---|
| | | | | ESI: |
| Ex. | Structure | Systematic Name | Building Blocks | m/z |
| B.46 | 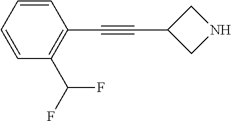 [SMILES: FC(F)c1ccccc1C#CC1CNC1] | 3-[2-[2- (difluoromethyl)phenyl] ethynyl]azetidine | 1-Bromo-2- difluoromethyl)ben- zene (CAS: 845866-82-2) | 208.1 [M + H]⁺ |

### Example B.36

### 3-(2-chloro-3-cyclopropylphenoxy)azetidine 4-methylbenzenesulfonate

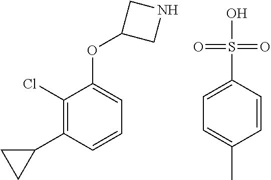

To a solution of tert-butyl 3-(2-chloro-3-cyclopropylphenoxy)azetidine-1-carboxylate (82.0 mg, 253 μmol) in EtOAc (603 μL) was added 4-methylbenzenesulfonic acid monohydrate (50.6 mg, 266 μmol). The reaction mixture was refluxed (80° C.) for 16 h. The reaction mixture was cooled and the solvent was evaporated to yield the title compound (100 mg, 94.8%). MS (ESI): m/z=224.1 [M-C₇H₈O₃S+H]⁺

### Step a) tert-butyl 3-(3-bromo-2-chlorophenoxy)azetidine-1-carboxylate

3-Bromo-2-chlorophenol (300 mg, 1.45 mmol) and tert-butyl 3-hydroxyazetidine-1-carboxylate (250 mg, 1.45 mmol) were dissolved in dry toluene (4.52 mL), followed by addition of 2-(tributyl-15-phosphaneylidene)acetonitrile (524 mg, 584 μL, 2.17 mmol). The reaction mixture was stirred at 100° C. for 1 h. The reaction mixture was diluted with EtOAc and washed with 1 M aq. NaHCO₃solution. The organic phase was collected and the aqueous phase underwent back-extraction with EtOAc. The combined organic layers were washed with Brine and dried over Na₂SO₄. The crude material was purified with SFC. Evaporation of the solvent gave the title compound (443 mg, 80.2%). MS (ESI): m/z=308.0 [M-C₄H₉+H]⁺

### Step b) tert-butyl 3-(2-chloro-3-cyclopropylphenoxy)azetidine-1-carboxylate

Tert-butyl-3-(3-bromo-2-chlorophenoxy)azetidine-1-carboxylate (355 mg, 930 μmol), cyclo-propylboronic acid (120 mg, 1.39 mmol) and K₂CO₃ (257 mg, 1.86 mmol) were dissolved in Dioxane (7.44 mL). The reaction mixture was degassed with a stream of Argon, followed by addition of H₂O (1.86 mL) and bis(triphenylphosphine)palladium(II)chloride (65.3 mg, 93.0 μmol). The reaction mixture was stirred at 100° C. for 18 h. The reaction mixture was diluted with EtOAc and washed with H₂O. The organic phase was collected and the aqueous phase underwent back-extraction with EtOAc. The combined organic layers were washed with brine and dried over Na₂SO₄. The crude material was purified with SFC. Evaporation of the solvent gave the title compound (82 mg, 26.7%). MS (ESI): m/z=268.1 [M-C₄H₉+H]⁺

In analogy to Example B.36, Examples B.37 in the following table were synthesized, using the respective commercially available phenols or hydroxy-pyridines.

| | | | | | MS, |
|---|---|---|---|---|---|
| | | | | Building | ESI: |
| Ex. | Structure | | Systematic Name | Blocks | m/z |
| B.37 | 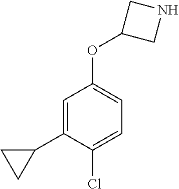 [SMILES: Clc1ccc(OC2CNC2)cc1C1CC1] | 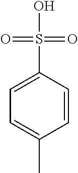 | 3-(4-chloro-3- cyclopropylphenoxy)aze- tidine 4- methylbenzenesulfonate | 3-bromo- 4- chloro- phenol (CAS: 13659- 24-0) | 224.1 [M − C₇H₈O₃S + H]⁺ |

### Example B.38

### 3-(azetidin-3-yloxy)-2-chloro-6-methylpyridine 4-methylbenzenesulfonate

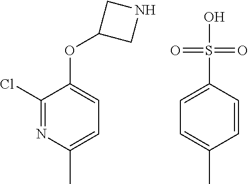

To a solution of tert-butyl 3-((2-chloro-6-methylpyridin-3-yl)oxy)azetidine-1-carboxylate (400 mg, 1.34 mmol) in EtOAc (3.19 mL) was added 4-methylbenzenesulfonic acid monohydrate (267 mg, 1.41 mmol). The reaction mixture was refluxed (80° C.) for 16 h. The reaction mixture was cooled and the solvent was evaporated to yield the title compound (509 mg, 97.4%). MS (ESI): m/z=199.1 [M-C₇H₈O₃S+H]⁺

### Step a) tert-butyl 6-((5-chloropyridin-3-yl)oxy)-2-azaspiro[3.3]heptane-2-carboxylate

2-chloro-6-methylpyridin-3-ol (300 mg, 2.09 mmol) and tert-butyl 3-hydroxyazetidine-1-carboxylate (362 mg, 2.09 mmol) were dissolved in dry Toluene (6.53 mL), followed by addition of 2-(tributyl-15-phosphaneylidene)acetonitrile (757 mg, 844 μL, 3.13 mmol). The reaction mixture was stirred at 100° C. for 1 h. The reaction mixture was diluted with EtOAc and washed with 1 M aq. NaHCO₃ solution. The organic phase was collected and the aqueous phase underwent back-extraction with EtOAc. The combined organic layers were washed with Brine and dried over Na₂SO₄. The crude material was purified with SFC. Evaporation of the solvent gave the title compound (400 mg, 60.90%). MS (ESI): m/z=299.2 [M-C₄H₉+H]⁺

In analogy to Example B.38, Examples B.39-B.42 in the following table were synthesized, using the respective commercially available phenols or pyridins.

| | | | | | MS, |
|---|---|---|---|---|---|
| | | | | Building | ESI: |
| Ex. | Structure | | Systematic Name | Blocks | m/z |
| B.39 | 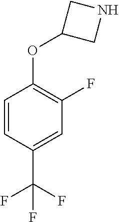 [SMILES: Fc1cc(C(F)(F)F)ccc1OC1CNC1] | 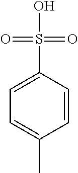 | 3-(2-fluoro-4- (trifluoromethyl)phenoxy)azeti- dine 4-methylbenzenesulfonate | 2-fluoro- 4- (trifluoro- methyl) phenol (CAS: 77227- 78-2) | 236.1 [M − C₇H₈O₃S + H]⁺ |
| B.40 | 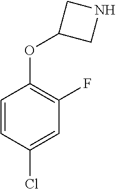 [SMILES: Fc1cc(Cl)ccc1OC1CNC1] | 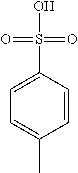 | 3-(4-chloro-2- flurophenoxy)azetidine 4- methylbenzenesulfonate | 4-chloro- 2- fluoro- phenol (CAS: 446-30- 0) | 202.1 [M − C₂HF₃O₂ + H]⁺ |
| B.41 | 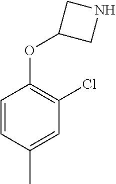 | 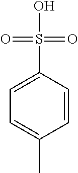 | 3-(2-chloro-4- methylphenoxy)azetidine 4- methylbenzenesulfonate | 2-chloro- 4- methyl- phenol (CAS: 6640-27- 3) | 198.1 [M − C₇H₈O₃S + H]⁺ |
| B.42 | 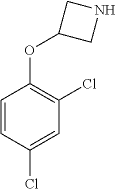 [SMILES: Clc1ccc(OC2CNC2)c(Cl)c1] | 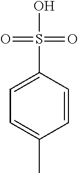 | 3-(2,4- dichlorophenoxy)azetidine 4- methylbenzenesulfonate | 2,4- dichloro- phenol (CAS: 120-83- 2) | 218.1 [M − C₇H₈O₃S + H]⁺ |

### Example B.49

### 4-(2-azaspiro[3.3]heptan-6-yloxy)-3-fluoro-benzonitrile; 4-methylbenzenesulfonic acid

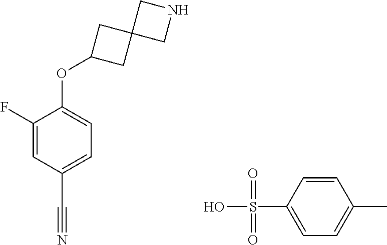 [SMILES: N#Cc1ccc(OC2CC3(CNC3)C2)c(F)c1 ; Cc1ccc(S(=O)(=O)O)cc1]

To an solution of tert-butyl 6-(4-cyano-2-fluorophenoxy)-2-azaspiro[3.3]heptane-2-carboxylate (1.00 g, 3.01 mmol) in ethyl acetate (35.6 mL) was added 4-methylbenzenesulfonic acid monohydrate (572 mg, 3.01 mmol) and the mixture was heated at reflux for 1 h. The solution was allowed to cool down to RT and was concentrated in vacuo. With addition of CH₂Cl₂ and Et₂O overnight at 4° C. it become solid. The crystals were isolated and washed with Et₂O and dried under high vacuum to yield the title compound (1.00 g, 90% purity, 74%) as a white solid. 233.1 [M-C₇H₈O₃S+H]⁺

### Step a) tert-butyl 6-(4-cyano-2-fluoro-phenoxy)-2-azaspiro[3.3]heptane-2-carboxylate

To a solution of 3-fluoro-4-hydroxybenzonitrile (2.83 g, 20.6 mmol), tert-butyl 6-hydroxy-2-azaspiro[3.3]heptane-2-carboxylate (4.00 g, 18.8 mmol) and triphenylphosphine (9.84 g, 37.5 mmol) in THF (93.8 mL) was added DIAD (7.58 g, 7.29 mL, 37.5 mmol) dropwise at 0° C. and the reaction was stirred at RT over night. The reaction mixture was diluted with sat. aq. NaHCO₃ solution and, extracted with EtOAc. The combined organics were dried over sodium sulfate, filtered and concentrated. The crude was purified by column chromatography eluting with 0-30% heptanes: EtOAc) to yield the title compound (6.7 g, impure, 107%) as a white solid. MS (ESI): m/z=277.2 [M-C₄H₉+H]⁺

### Example B.51

### 6-[[1-(trifluoromethyl)cyclopropyl]methoxy]-2-azaspiro[3.3]heptane 4-methylbenzenesulfonate

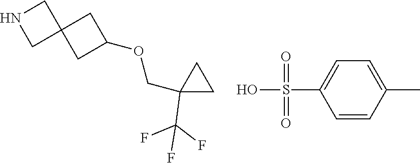

A solution of 6-[[1-(trifluoromethyl)cyclopropyl]methoxy]-2-azaspiro[3.3]heptane-2-carboxylic acid tert-butyl ester (633 mg, 1.89 mmol) in ethyl acetate (20 mL) was treated with p-toluenesulfonic acid monohydrate (366 mg, 1.93 mmol), at 23° C. The mixture was then heated to 80° C. for 18 h, before being cooled down to 23° C. and evaporated, to give 6-[[1-(trifluoromethyl)cyclopropyl]methoxy]-2-azaspiro[3.3]heptane 4-methylbenzenesulfonate (769 mg, 95.0%) as light yellow solid. MS (ESI): m/z=236.2 [M-C₇H₈O₃S+H]⁺

### Step a) 6-[[ ]-(trifluoromethyl)cyclopropyl]methoxy]-2-azaspiro[3.3]heptane-2-carboxylic acid tert-butyl ester

A solution of 6-hydroxy-2-azaspiro[3.3]heptane-2-carboxylic acid tert-butyl ester (500 mg, 2.34 mmol) in N,N-dimethylformamide, extra dry (10 mL) was treated with 1-(bromomethyl)-1-(trifluoromethyl)cyclopropane (476 mg, 2.34 mmol), at 23° C. under Ar. The mixture was stirred for another 30 min at this temperature, before being heated to 80° C. and stirred for 21.5 h. The mixture was then cooled down to 23° C., diluted with EtOAc, and the organic layer was washed with 1 M NaHCO₃ solution (1×), water (2×), and brine (1×). The organic layer was then dried over Na₂SO₄, filtered, and evaporated, to give 6-[[1-(trifluoromethyl)cyclopropyl]methoxy]-2-azaspiro[3.3]heptane-2-carboxylic acid tert-butyl ester (633 mg, 73%) as a crude colorless oil which was used directly without further purification. MS (ESI): m/z=280.2 [M+H−tBu]⁺

### Example B.53

### 3-(azetidin-3-yl)-5-[1-(trifluoromethyl)cyclopropyl]-1,2,4-oxadiazole;4-methylbenzenesulfonic acid salt

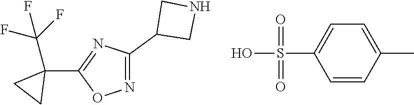 [SMILES: FC(F)(F)C1(c2nc(C3CNC3)no2)CC1 ; Cc1ccc(S(=O)(=O)O)cc1]

To a solution of tert-butyl 3-[5-[1-(trifluoromethyl)cyclopropyl]-1,2,4-oxadiazol-3-yl]azetidine-1-carboxylate (1200 mg, 3.6 mmol) in ethyl acetate (20 mL) was added p-toluenesulfonic acid (744 mg, 4.32 mmol), the mixture was stirred at 80° C. for 12 h. The mixture was cooled to room temperature, concentrated to give the title compound (1315 mg, 3.24 mmol, 89.7% yield) as a brown waxy solid. MS (ESI): m/z=234.4 [M-C₇H₈O₃S+H]⁺

### Step a) tert-butyl 3-(N-hydroxycarbamimidoyl)azetidine-1-carboxylate

To a solution of hydroxylamine hydrochloride (1.53 g, 22.0 mmol) and 1-Boc-3-cyanoazetidine (2.0 g, 11.0 mmol) in methanol (20 mL) and water (20 mL) was added sodium carbonate (2.33 g, 22.0 mmol) and the mixture was stirred at 50° C. for 12 h. The mixture was filtered, and the filtrate was concentrated under vacuum to remove the ethanol, then the residual mixture was extracted with EtOAc (50 mL×2). The combined organic phase was dried over Na₂SO₄ and concentrated to give the title compound (1.8 g, 8.36 mmol, 76.2% yield) as a light yellow solid. MS (ESI): m/z=160.2 [M-C₄H₈+H]⁺

### Step b) 3-[(Z)—N′-[ ]-(trifluoromethyl)cyclopropanecarbonyl]oxycarbamimidoyl]azetidine-]-carboxylate

To a solution of 1-(trifluoromethyl)cyclopropane-1-carboxylic acid (1432 mg, 9.29 mmol), DIPEA (3603 mg, 27.9 mmol) and O-(7-Azabenzotriazol-1-yl)-N,N,N′,N′-tetramethyluronium hexafluorophosphate (4240 mg, 11.2 mmol) in DCM (40 mL) was added tert-butyl 3-(N-hydroxycarbamimidoyl)azetidine-1-carboxylate (2000 mg, 9.29 mmol), then the reaction mixture was stirred at 20° C. for 16 h. The mixture was evaporated and purified by reverse flash chromatography (FA) to give the title compound (2600 mg, 7.4 mmol, 79.7% yield) as light brown oil. MS (ESI): m/z=296.3 [M-C₄H₈+H]⁺

### Step c) tert-butyl 3-[5-[ ]-(trifluoromethyl)cyclopropyl]-1,2,4-oxadiazol-3-yl]azetidine-1-carboxylate

To a solution of tert-butyl 3-[(Z)—N′-[1-(trifluoromethyl) cyclopropanecarbonyl]oxycarbamimidoyl]azetidine-1-carboxylate (1500 mg, 4.27 mmol) in ethanol (37.5 mL) and water (37.5 mL) was added KOAc (838 mg, 8.54 mmol). The mixture was stirred at 80° C. for 12 h, then the mixture was concentrated and diluted with EtOAc (50 mL), washed with water and brine, dried over Na₂SO₄ and concentrated. The residue was purified by reverse flash chromatography to give the title compound (1250 mg, 3.75 mmol, 87.8% yield) as light yellow oil. MS (ESI): m/z=278.4 [M-C₄H₈+H]⁺

### Example B.54

### 1-(azetidin-3-yl)-4-(2,2,2-trifluoroethoxy)pyrazole; 4-methylbenzenesulfonic acid salt

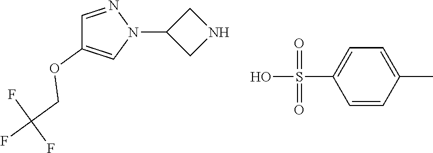

To a solution of tert-butyl 3-[4-(2,2,2-trifluoroethoxy)pyrazol-1-yl]azetidine-1-carboxylate (880 mg, 2.74 mmol) in Ethyl acetate (25 mL) was added p-toluenesulfonic acid (566 mg, 3.29 mmol), the mixture was stirred at 80° C. for 12 h. The mixture was cooled to 20° C. and stirred for another 2 h, filtered and the cake washed with EtOAc (20 mL). The filter cake was collected and dried to give 1-(azetidin-3-yl)-4-(2,2,2-trifluoroethoxy)pyrazole; 4-methylbenzenesulfonic acid (855 mg, 2.17 mmol, 79.1% yield) as off-white solid. MS (ESI): m/z=234.4 [M-C₇H₈O₃S+H]⁺

### Step a) tert-butyl 3-(4-hydroxypyrazol-1-yl)azetidine-1-carboxylate

To a cold (0° C., ice bath) solution of tert-butyl 3-[4-(4,4,5,5-tetramethyl-1,3,2-dioxaborolan-2-yl)pyrazo]-1-yl]azetidine-1-carboxylate (80.0 mg, 0.230 mmol) in THF (2 mL) was slowly added a solution of sodium hydroxide (18.3 mg, 0.460 mmol) in water (0.200 mL), followed by hydrogen peroxide (51.9 mg, 0.460 mmol). The reaction mixture was stirred at 0-20° C. for 3 h. The mixture was carefully neutralized with 1 N HCl and diluted with EtOAc (5 mL). The aqueous layer was extracted with EtOAc (5 mL three times). The combined organic layers were dried (Na₂SO₄) and concentrated under reduced pressure to afford the title compound (54 mg, 0.230 mmol, 98.5% yield) as light yellow oil which was used directly without further purification. MS (ESI): m/z=184.5 [M-C₄H₈+H]⁺

### Step b) tert-butyl 3-[4-(2,2,2-trifluoroethoxy)pyrazol-1-yl]azetidine-1-carboxylate

To a solution of tert-butyl 3-(4-hydroxypyrazol-1-yl)azetidine-1-carboxylate (1.30 g, 5.43 mmol) in DMF (30 mL) was added NaH (0.26 g, 6.52 mmol) at 0° C., and the mixture stirred for 30 min. Then 2,2,2-trifluoroethyl trifluoromethanesulfonate (1.28 mL, 8.15 mmol) was added dropwise at 0° C., and the mixture was stirred at 20° C. for 2 h. The mixture was diluted with water (300 mL) and extracted with EtOAc (100 mL three times). The combined organic phase was washed with brine (100 mL), dried with Na₂SO₄, concentrated and purified by reversed flash chromatography (0.05% v/v FA condition) to give the title compound (982 mg, 3.06 mmol, 52.2% yield) as a yellow oil. MS (ESI): m/z=266.0 [M-C₄H₈+H]⁺

### Example B.55

### 2-(azetidin-3-yl)-5-[1-(trifluoromethyl)cyclopropyl]-1,3,4-oxadiazole; 2,2,2-trifluoroacetic acid salt

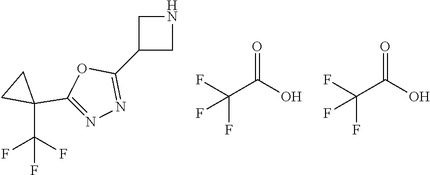

To a solution of tert-butyl 3-[5-[1-(trifluoromethyl)cyclopropyl]-1,3,4-oxadiazol-2-yl]azetidine-1-carboxylate (1150 mg, 3.45 mmol) in DCM (10 mL) was added trifluoroacetic acid (2.0 mL, 59.7 mmol), and the mixture was stirred at 20° C. for 12 h. The mixture was concentrated to give the title compound (1837 mg, 3.98 mmol, 108.5% yield) as light yellow oil. MS (ESI): m/z=234.4 [M-2TFA+H]⁺

### Step a) tert-butyl 3-(hydrazinecarbonyl)azetidine-1-carboxylate

1-BOC-azetidine-3-carboxylic acid (5.0 g, 24.9 mmol) was suspended in DCM (15 mL) and N,N′-Carbonyldiimidazole (4.83 g, 29.8 mmol) was added in portions. The resulting mixture was stirred at 20° C. for 30 min and then added dropwise to a solution of hydrazine hydrate (1.87 g, 37.3 mmol) in DCM (5 mL). After the addition was complete, the mixture was stirred for 12 h at 20° C. The reaction mixture was washed with saturated aqueous Na₂CO₃ solution, brine, dried over Na₂SO₄ and concentrated under vacuum to give tert-butyl 3-(hydrazinecarbonyl)azetidine-1-carboxylate (3.6 g, 16.7 mmol, 67.3% yield) as a colorless oil. MS (ESI): m/z=238.4 [M+Na]⁺

### Step b) 3-[[[ ]-(trifluoromethyl)cyclopropanecarbonyl]amino]carbamoyl]azetidine-]-carboxylate

To a solution of 1-(trifluoromethyl)cyclopropane-1-carboxylic acid (1432 mg, 9.29 mmol), DIPEA (3603 mg, 27.9 mmol) and O-(7-Azabenzotriazol-1-yl)-N,N,N′,N′-tetramethyluronium hexafluorophosphate (4240 mg, 11.2 mmol) in DCM (40 mL) was added tert-butyl 3-(hydrazinecarbonyl)azetidine-1-carboxylate (2000 mg, 9.29 mmol), then stirred at 20° C. for 16 h. The mixture was evaporated and purified by reverse flash (FA) to give the title compound (2100 mg, 5.98 mmol, 67.8% yield) as light yellow oil. MS (ESI): m/z=296.3 [M-C₄H₈+H]⁺

### Step c) tert-butyl 3-[5-[ ]-(trifluoromethyl)cyclopropyl]-1,3,4-oxadiazol-2-yl]azetidine-1-carboxylate

To a suspension of tert-butyl 3-[[[1-(trifluoromethyl)cyclopropanecarbonyl]amino]carbamoyl]azetidine-1-carboxylate (1500 mg, 4.27 mmol) in MeCN (30 mL) was added DIPEA (4.46 mL, 25.6 mmol) and triphenylphosphine (2016 mg, 7.69 mmol), followed after 5 min by hexachloroethane (1517 mg, 6.4 mmol). After stirring the mixture at 20° C. for 12 h, the solvent was removed in vacuo and the residue was purified by silica gel column (eluting with PE:EtOAc=10:1 to 5:1) to give the title compound (1200 mg, 3.6 mmol, 84.3% yield) as a white solid. MS (ESI): m/z=278.4 [M-C₄H₈+H]⁺

### Example B.56

### 5-methyl-6-(2,2,2-trifluoro-1,1-dimethyl-ethoxy)pyridine-3-carboxylic acid

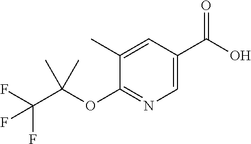 [SMILES: Cc1cc(C(=O)O)cnc1OC(C)(C)C(F)(F)F]

A solution of methyl 5-methyl-6-(2,2,2-trifluoro-1,1-dimethyl-ethoxy)pyridine-3-carboxylate (710 mg, 2.56 mmol) in MeOH (1.0 mL, 20.5 mmol) and tetrahydrofuran (30 mL), lithium hydroxide (286 mg, 12.0 mmol) in water (30 mL) was added, the reaction mixture was stirred at 25° C. for 2 h. The reaction mixture was evaporated under reduced pressure and purified with reversed Flash chromatography to yield the title compound (350 mg, 1.33 mmol, 51.9% yield) as a dark brown solid. MS (ESI): m/z=264.6 [M+H]⁺

### Step a) 5-bromo-3-methyl-2-(2,2,2-trifluoro-],1-dimethyl-ethoxy)pyridine

Under N₂ atmosphere, NaH (937 mg, 23.4 mmol) was added to a solution of 2-trifluoromethyl-2-propanol (2000 mg, 15.6 mmol) in DMF (87.5 mL), and stirred at 0° C. for 1 h, then 5-bromo-2-fluoro-3-methylpyridine (3264 mg, 17.1 mmol) was added and stirred at 100° C. for 12 h. The reaction mixture was quenched with saturated aq. NH₄Cl solution and then extracted with EtOAc, the combined organic layer was evaporated and purified with MPLC to give 5-bromo-3-methyl-2-(2,2,2-trifluoro-1,1-dimethyl-ethoxy)pyridine (2840 mg, 9.53 mmol, 61.0% yield) as a colorless oil. MS (ESI): m/z=298.5 [M+H]⁺

### Step b) methyl 5-methyl-6-(2,2,2-trifluoro-],1-dimethyl-ethoxy)pyridine-3-carboxylate

A mixture of 5-bromo-3-methyl-2-(2,2,2-trifluoro-1,1-dimethyl-ethoxy)pyridine (1400 mg, 4.7 mmol), Pd(dppf)Cl₂ (344 mg, 0.470 mmol) and TEA (1426 mg, 14.1 mmol) in methanol (42 mL) was purged with carbon monoxide (50 psi) and stirred at 80° C. for 16 h. The mixture was filtered and the filtrate was concentrated under vacuum. The residue was purified by column phromatography (PE:EA=3:1) to give crude methyl 5-methyl-6-(2,2,2-trifluoro-1,1-dimethyl-ethoxy)pyridine-3-carboxylate (710 mg, 2.56 mmol, 54.5% yield) as light yellow oil. MS (ESI): m/z=278.6[M+H]⁺

### Example B.57

### 3-(5-(tert-butyl)-1,2,4-oxadiazol-3-yl)bicyclo[1.1.1]pentane-1-carboxylic acid

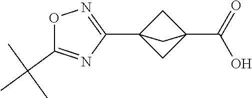 [SMILES: CC(C)(C)c1nc(C23CC(C(=O)O)(C2)C3)no1]

To a solution of methyl 3-(5-(tert-butyl)-1,2,4-oxadiazol-3-yl)bicyclo[1.1.1]pentane-1-carboxylate (105 mg, 419 μmol) in THF (699 μl) MeOH (699 μl) and Water (699 μl) was added lithium hydroxide hydrate (52.8 mg, 1.26 mmol, Eq: 3). The mixture was stirred at room temperature for 6 hours. The mixture was acidified (pH=2) using 2N HCl. The aqueous phase was extracted with ethyl acetate (3×5 ml). The combined organic phases were dried over MgSO4 and evaporated to dryness. 3-(5-(tert-butyl)-1,2,4-oxadiazol-3-yl)bicyclo[1.1.1]pentane-1-carboxylic acid (55 mg, 189 μmol, 44.9% yield) was obtained as a white solid. MS (ESI): m/z=237.2 [M+H]⁺

### Step a) methyl 3-(5-(tert-butyl)-1,2,4-oxadiazol-3-yl)bicyclo[1.1.1]pentane-]-carboxylate

To a solution of methyl 3-cyanobicyclo[1.1.1]pentane-1-carboxylate (CAS: 156329-62-3) (750 mg, 4.96 mmol) in ethanol (16.5 mL) was added hydroxylamine (492 mg, 439 μL, 7.44 mmol). The mixture was heated to reflux for 5 h. The volatiles were removed under reduced pressure. The residue was redissolved in DMF (5.5 mL). Pivaloyl chloride (718 mg, 733 μL, 5.95 mmol) was added followed by TEA (1.51 g, 2.07 mL, 14.9 mmol) and a white precipitate formed. The mixture was heated to 125° C. for 24 h. The reaction mixture was diluted with ethyl acetate and washed with water. The aqueous phase was extracted with ethyl acetate twice and the combined organic phases were washed with water (acidified with 2 N HCl, pH=3) and brine and dried over MgSO₄. The solvent was removed under reduced pressure. The crude product was purified by column chromatography using heptane:ethyl acetate (9:1) as solvent.

The title compound (0.624 g, 2.09 mmol, 42.2% yield) was obtained as a white solid. MS (ESI): m/z=251.2 [M+H]⁺

### Example B.58

### 4-(azetidin-3-yl)-1-[1-(trifluoromethyl)cyclopropyl]triazole; 4-methylbenzenesulfonic acid

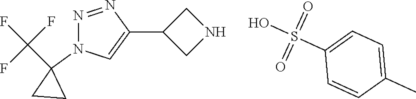 [SMILES: FC(F)(F)C1(n2cc(C3CNC3)nn2)CC1 ; Cc1ccc(S(=O)(=O)O)cc1]

To a solution of tert-butyl 3-[1-[1-(trifluoromethyl)cyclopropyl]triazol-4-yl]azetidine-1-carboxylate (400 mg, 1.2 mmol) in ethyl acetate (10 mL) was added p-toluenesulfonic acid (249 mg, 1.44 mmol), the mixture was stirred at 80° C. for 12 h. The mixture was cooled to room temperature, filtered and the filter cake was collected to give the title compound (420 mg, 1.04 mmol, 86% yield) as white solid. MS (ESI): m/z=233.1 [M+H]⁺

### Step a) tert-butyl 3-[ ]−[ ]-(trifluoromethyl)cyclopropyl]triazol-4-yl]azetidine-]-carboxylate

1-(trifluoromethyl)cyclopropanamine hydrochloride (950 mg, 5.88 mmol) was added to a suspension of copper(II) sulfate pentahydrate (147 mg, 0.59 mmol), potassium carbonate (2032 mg, 14.7 mmol) and 1H-imidazole-1-sulfonyl azide hydrochloride (1480 mg, 7.06 mmol) in methanol (19 mL), the mixture was stirred at 25° C. for 12 h, then tert-butyl 3-ethynylazetidine-1-carboxylate (533 mg, 2.94 mmol), copper powder (374 mg, 5.88 mmol), acetic acid (1.9 mL, 33.2 mmol) and aq. copper(II) sulfate (1.9 mL, 5.88 mmol) and THF (38 mL) was added, the mixture was stirred at 25° C. for another 2 h. The mixture was filtered and the filtrate was concentrated. The residue was redissolved in EtOAc and washed with aq. NH₃·H₂O (10%) and brine, then dried over Na₂SO₄, concentrated and the residue was purified by reverse flash chromatography (FA) to give the title compound (440 mg, 1.32 mmol, 22.5% yield) as awhite solid. MS (ESI): m/z=277.4 [M-C₄H₈+H]⁺

### Example B.59

### 5-(azetidin-3-yl)-3-[1-(trifluoromethyl)cyclopropyl]-1,2,4-oxadiazole; 4-methylbenzenesulfonic acid

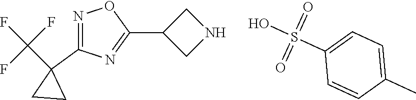 [SMILES: FC(F)(F)C1(c2noc(C3CNC3)n2)CC1 ; Cc1ccc(S(=O)(=O)O)cc1]

To a solution of tert-butyl 3-[3-[1-(trifluoromethyl)cyclopropyl]-1,2,4-oxadiazol-5-yl]azetidine-1-carboxylate (310 mg, 0.93 mmol) in rthyl acetate (7 mL) was added p-toluenesulfonic acid (192 mg, 1.12 mmol), the mixture was stirred at 80° C. for 12 h. The mixture was cooled to room temperature, filtered and the filter cake was collected to give the title compound (341 mg, 0.84 mmol, 90% yield) as white solid. MS (ESI): m/z=234.4 [M+H]⁺

### Step a) 1-(trifluoromethyl)cyclopropanecarboxamide

A solution of 1-(trifluoromethyl)cyclopropane-1-carboxylic acid (2000 mg, 13.0 mmol) in DCM (20 mL) was treated with oxalyl chloride (2142 mg, 16.9 mmol) and 1 drop of DMF, stirred for 1 h at 20° C., then added drop-wise to a solution of aq. NH₄0H (20.0 mL, 300 mmol) in THF (20 mL) and stirred at 20° C. for 12 h. The solids were removed via filtration through diatomaceous earth and rinsed well with DCM/THF (4:1, 50 mL). The filtrate was saturated with solid NaCl, extracted with DCM/THF (4:1, 50 mL three times) and the combined organics were dried over Na₂SO₄ and concentrated to dryness to afford the title compound (1430 mg, 9.34 mmol, 72% yield) as light yellow solid. MS (ESI): m/z=154.6 [M+H]⁺

### Step b) N′-hydroxy-]-(trifluoromethyl)cyclopropanecarboxamidine

To a solution of 1-(trifluoromethyl)cyclopropanecarboxamide (2100 mg, 13.7 mmol) in THF (25 mL) was added trifluoroacetic anhydride (9.69 mL, 68.6 mmol), the mixture then was stirred for 12 h at 65° C. under a nitrogen atmosphere. After cooling to room temperature potassium carbonate (17060 mg, 123 mmol), hydroxylamine hydrochloride (2860 mg, 41.2 mmol) and methanol (160 mL) are added and the reaction mixture was heated at 65° C. for 12 h. The mixture was concentrated and the residue was dissolved in EtOAc, and washed with water and brine. The organic phase was dried over Na₂SO₄, concentrated to give the title compound (1120 mg, 6.66 mmol, 49% yield) as light yellow oil. MS (ESI): m/z=169.5 [M+H]⁺

### Step c) 03-[[amino-[ ]-(trifluoromethyl)cyclopropyl]methylene]amino] 01-tert-butyl azetidine-1, 3-dicarboxylate

To a solution of 1-Boc-azetidine-3-carboxylic acid (1100 mg, 5.47 mmol), DIPEA (2120 mg, 16.4 mmol) and propylphosphonicanhydride solution 50 wt. % in ethyl acetate (3774 mg, 8.2 mmol) in DCM (22 mL) was added N′-hydroxy-1-(trifluoromethyl)cyclopropanecarboxamidine (1103 mg, 6.56 mmol), then the mixture was stirred at 20° C. for 12 h. The reaction mixture was washed with water and brine, dried over Na₂SO₄ and concentrated, and the residue was purified by reverse flash chromatography (FA) to give the title compound (540 mg, 1.54 mmol, 30% yield) as a light yellow solid. MS (ESI): m/z=296.3 [M-C₄H₈+H]⁺

### Step d) tert-butyl 3-[3-[ ]-(trifluoromethyl)cyclopropyl]-1,2,4-oxadiazol-5-yl]azetidine-1-carboxylate

A solution of 03-[[amino-[1-(trifluoromethyl)cyclopropyl]methylene]amino] 01-tert-butyl azetidine-1,3-dicarboxylate (540 mg, 1.54 mmol) and NaOAc (252 mg, 3.07 mmol) in ethanol (10 mL) and water (10 mL) was stirred at 80° C. for 12 h. The reaction was concentrated and the residue was dissolved with EtOAc (10 mL), washed with water and brine, the organic phase was dried over Na₂SO₄ and concentrated. The residue was purified by reverse flash chromatography (FA) to give the title compound (310 mg, 0.93 mmol, 61% yield) as light yellow oil. MS (ESI): m/z=278.4 [M-C₄H₈+H]⁺

### Example B.60

### 4-(azetidin-3-yl)-1-[3-(trifluoromethyl)oxetan-3-yl]triazole; 4-methylbenzenesulfonic acid

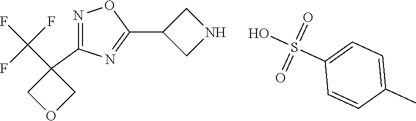 [SMILES: FC(F)(F)C1(c2noc(C3CNC3)n2)COC1 ; Cc1ccc(S(=O)(=O)O)cc1]

To a solution of tert-butyl 3-[1-[3-(trifluoromethyl)oxetan-3-yl]triazol-4-yl]azetidine-1-carboxylate (120 mg, 0.34 mmol) in ethyl acetate (3 mL) was added p-toluenesulfonic acid (71.2 mg, 0.41 mmol) and the mixture was stirred at 80° C. for 12 h. The mixture was cooled to room temperature, filtered and the filter cake was collected to give the title compound (110 mg, 0.26 mmol, 76% yield) as an off-white solid. MS (ESI): m/z=249.1 [M+H]⁺

### Step a) tert-butyl 3-[ ]−[3-(trifluoromethyl)oxetan-3-yl]triazol-4-yl]azetidine-1-carboxylate

3-(trifluoromethyl)oxetan-3-amine hydrochloride (400 mg, 2.25 mmol) was added to a suspension of copper(II) sulfate pentahydrate (56.3 mg, 0.230 mmol), potassium carbonate (778 mg, 5.63 mmol) and 1H-imidazole-1-sulfonyl azide hydrochloride (567 mg, 2.7 mmol) in methanol (8 mL). The mixture was stirred at 25° C. for 12 h, then tert-butyl 3-ethynylazetidine-1-carboxylate (163 mg, 0.900 mmol), copper powder (143 mg, 2.25 mmol), acetic acid (0.8 mL, 0.280 mmol) and aq. copper(II) sulfate (0.8 mL, 2.25 mmol) and THF (16 mL) was added, the mixture was stirred at 25° C. for another 2 h. The mixture was filtered and the filtrate was concentrated, the residue was redissolved in EtOAc and washed with aq. NH₃·H₂O (10%) and brine, then dried over Na₂SO₄, concentrated and the residue was purified by reverse flash chromatography (FA) to give the title compound (80 mg, 0.230 mmol, 10% yield) as an off-white solid. MS (ESI): m/z=249.4 [M-Boc+H]⁺

### Example B.61

### 4-(5-tert-butyl-1,3,4-oxadiazol-2-yl)benzoic acid

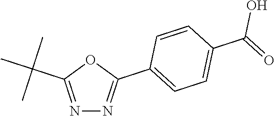 [SMILES: CC(C)(C)c1nnc(-c2ccc(C(=O)O)cc2)o1]

To a solution of methyl 4-(5-tert-butyl-1,3,4-oxadiazol-2-yl)benzoate (1.5 g, 5.76 mmol) in THF (10 mL) was added 2 M NaOH (11.5 mL, 23.0 mmol) and methanol (10 mL), the reaction was stirred at 25° C. for 12 h. The reaction was concentrated in vacuum to remove the solvent and acidified with 1 M HCl to pH=6. The mixture was extracted with EA (50 mL×3), the combined organic layer was washed with water (30 mL×2) and brine (20 mL), dried over sodium sulfate and concentrated in vacuum to yield the title compound (1.3 g, 5.28 mmol, 92% yield) as light yellow solid. MS (ESI): m/z=247.2 [M+H]⁺

### Step a) 4-bromo-N-[(E)-2,2-dimethylpropylideneamino]benzamide

A mixture of trimethylacetaldehyde (4.41 g, 51.2 mmol) and 4-bromobenzhydrazide (CAS: 5933-32-4) (10.0 g, 46.5 mmol) in ethanol (160 mL) was stirred at 80° C. for 12 h. The mixture was concentrated to give the title compound (13.4 g, 47.3 mmol, 101% yield) as light yellow solid. MS (ESI): m/z=284.1 [M+H]⁺

### Step b) 2-(4-bromophenyl)-5-tert-butyl-1,3,4-oxadiazole

To a solution of 4-bromo-N-[(E)-2,2-dimethylpropylideneamino]benzamide (7000 mg, 24.7 mmol) and Cs₂CO₃ (24.1 g, 74.2 mmol) in DMSO (167 mL) was added iodine (12.5 g, 49.4 mmol), and the mixture was stirred at 100° C. for 12 h. The mixture was poured into aq. Na₂SO₃ (150 mL) and extracted with EA (50 mL×3). The combined organic phase was washed with brine and dried over Na₂SO₄, filtered and the filtrate was concentrated to give the title compound (6.9 g, 24.5 mmol, 99% yield) as an orange oil. MS (ESI): m/z=281.0 [M+H]⁺

### Step c) methyl 4-(5-tert-butyl-1,3,4-oxadiazol-2-yl)benzoate

To a mixture of 2-(4-bromophenyl)-5-tert-butyl-1,3,4-oxadiazole (3000 mg, 10.7 mmol) and triethylamine (2.97 mL, 21.3 mmol) in methanol (157 mL) and were added Pd(dppf)Cl₂ (781 mg, 1.07 mmol) at 20° C. Then the mixture was purged with carbon monoxide (10.7 mmol) (50 psi) and stirred at 80° C. for 15 h. The mixture was filtered and the filtrate was concentrated in vacuo. The residue was purified by silica gel chromatography (petroleum ether: ethyl acetate 10:1 to 4:1) to give the title compound (2.5 g, 9.6 mmol, 90% yield) as a light yellow solid. MS (ESI): m/z=261.1 [M+H]⁺

### Example B.62

### 4-(1-tert-butylpyrazol-4-yl)benzoic acid

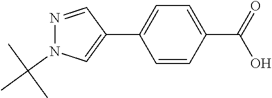 [SMILES: CC(C)(C)n1cc(-c2ccc(C(=O)O)cc2)cn1]

A solution of methyl 4-(1-tert-butylpyrazol-4-yl)benzoate (850 mg, 3.29 mmol) in THF (10 mL) was added Sodium hydroxide (526 mg, 13.2 mmol) in water (10 mL), the mixture was stirred at 20° C. for 1 h. The mixture was concentrated to remove THF, and the residual water phase was extracted with EA (30 mL×3). The aqueous phase was acidified with 1 M HCl to bring the pH to 3-4, then was extracted with EA (30 mL×3),the organic phase washed with brine, dried over Na₂SO₄ and concentrated to give the title compound (780 mg, 3.19 mmol, 93% yield) as a white solid. MS (ESI): m/z=245.1 [M+H]⁺

### Step a) methyl 4-(1-tert-butylpyrazol-4-yl)benzoate

To a solution of 4-bromo-1-tert-butyl-pyrazole (1000 mg, 4.92 mmol) and 4-methoxycarbonylphenylboronic acid (1063 mg, 5.91 mmol), sodium carbonate (1.57 g, 14.7 mmol) in 1,4-dioxane (50 mL) and water (5 mL) was added TETRAKIS[TRIPHENYLPHOSPHINE]PALLADIUM(0) (569 mg, 0.490 mmol), the mixture was stirred at 110° C. under N₂ atmosphere for 12 h. The reaction mixture was filtered and the filtrate was concentrated. The residue was triturated in a mixture of petroleum ether:ethyl acetate=3:1, filtered to collect the solid, and dried in vacuum to give the title compound (950 mg, 3.68 mmol, 75% yield) as a grey solid. MS (ESI): m/z=259.1 [M+H]⁺

### Example D.1

### [3-(2-azaspiro[3.3]heptan-6-ylmethyl)phenyl]-imino-oxo-(trifluoromethyl)-λ⁶-sulfane; 4-methylbenzenesulfonic acid

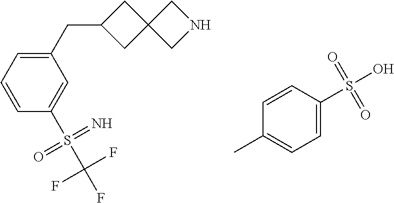 [SMILES: N=S(=O)(c1cccc(CC2CC3(CNC3)C2)c1)C(F)(F)F ; Cc1ccc(S(=O)(=O)O)cc1]

A mixture of p-toluenesulfonic acid (1.18 g, 6.83 mmol) and tert-butyl 6-[[3-(trifluoromethylsulfonimidoyl)phenyl]methyl]-2-azaspiro[3.3]heptane-2-carboxylate (1.3 g, 3.11 mmol) in ethyl acetate (30 mL) was stirred at 40° C. for 24 h. After the completion of the reaction, the reaction mixture was concentrated and purified by HPLC to afford the title compound (339 mg, 0.690 mmol, 15.6% yield) as brown viscous oil. MS (ESI): m/z=319.0 [M-TsOH+H]⁺

### Step a) tert-butyl 6-[(4,4,5,5-tetramethyl-1, 3,2-dioxaborolan-2-yl)methylene]-2-azaspiro[3.3]heptane-2-carboxylate

A mixture of 2,2,6,6-tetramethylpiperidine (95.9 mL, 568 mmol) in THF (750 mL) was cooled to −30° C. under a N₂ atmosphere. n-BuLi (227 mL, 568 mmol) was added dropwise, and the reaction mixture was stirred at the same temperature for 30 min. Next, the reaction was cooled to −60° C., and a solution of 4,4,5,5-tetramethyl-2-[(4,4,5,5-tetramethyl-1,3,2-dioxaborolan-2-yl)methyl]-1,3,2-dioxaborolane (136 g, 506 mmol) in THF (750 mL) was added dropwise. After stirring for 30 min, a solution of tert-butyl 6-oxo-2-azaspiro[3.3]heptane-2-carboxylate (100 g, 473 mmol) in THF (300 mL) was added in dropwise at −60° C. The reaction mixture was allowed to slowly warm up to 25° C. and stirred at 25° C. for 12 h. The mixture was added H₂O (8.0 mL) slowly. Extraction with EtOAc and purification (SiO₂; PE/EtOAc) gave the title compound (220 g, approx 69% yield per batch) as a white solid. ¹H NMR (400 MHz, CHLOROFORM-d) δ=5.21-5.16 (m, 1H), 3.99-3.89 (m, 4H), 3.13-2.90 (m, 4H), 1.46-1.41 (m, 9H), 1.26-1.20 ppm (m, 13H).

### Step b) tert-butyl 6-[[3-(trifluoromethylsulfonimidoyl)phenyl]methylene]-2-azaspiro[3.3]heptane-2-carboxylate

(3-Bromophenyl)-imino-oxo-(trifluoromethyl)-λ6-sulfane (2.47 g, 8.59 mmol), tert-butyl 6-[(4,4,5,5-tetramethyl-1,3,2-dioxaborolan-2-yl)methylene]-2-azaspiro[3.3]heptane-2-carboxylate (2.4 g, 7.16 mmol), 1,1′-bis(diphenylphosphino)ferrocene-palladium(II)dichloride dichloromethane complex (1.17 g, 1.43 mmol) and potassium carbonate (1.98 g, 14.3 mmol) were dissolved in 1,4-Dioxane (40 mL) and water (8 mL). The reaction mixture was heated to 120° C. under argon for 16 h. The reaction mixture was concentrated under reduced pressure.

The residue was partitioned between ethyl acetate and water. The organic layer was washed with brine. The extract was dried over sodium sulfate, filtered through a thin layer of silica gel and evaporated. The crude product was purified by column chromatography to afford the title compound (1 g, 31.9% yield) as a light yellow solid. MS (ESI): m/z=361.0 [M−tBu+H]⁺.

### Step c) tert-butyl 6-[[3-(trifluoromethylsulfonimidoyl)phenyl]methyl]-2-azaspiro[3.3]heptane-2-carboxylate

A mixture of tert-butyl 6-[[3-(trifluoromethylsulfonimidoyl)phenyl]methylene]-2-azaspiro[3.3]heptane-2-carboxylate (1.3 g, 3.12 mmol) and palladium on carbon (10%) (0.16 mL, 1.56 mmol) in EtOAc (35 mL) was stirred in an autoclave for 24 h under 30 bar of H₂. Then the reaction mixture was filtered and concentrated to afford the title compound (1.3 g, 96.5% yield) as a grey oil. MS (ESI): m/z=319.0 [M-Boc+H]⁺.

In analogy to Example D.1, the following building blocks were generated using the relevant (hetero)aryl bromide or iodide building block for the Suzuki coupling in Step b. In some cases, alternative salts (e.g. trifluoroacetate, ditosylate, hydrochloride) were also used. To introduce different spiro-ring systems further building block substitutions can be made, for example Example D.25 used tert-butyl 7-oxo-2-azaspiro[3.5]nonane-2-carboxylate (CAS: 1363381-22-9) in place of tert-butyl 6-oxo-2-azaspiro[3.3]heptane-2-carboxylate in Step a), and Example D.26 and D.47 used tert-butyl 6-oxo-2-azaspiro [3.4] octane-2-carboxylate (CAS: 1363382-39-1) in place of tert-butyl 6-oxo-2-azaspiro[3.3]heptane-2-carboxylate in Step a).

| | | | | | MS, |
|---|---|---|---|---|---|
| | | | | Building | ESI: |
| Ex. | Structure | | Systematic Name | Blocks | m/z |
| D.2 | 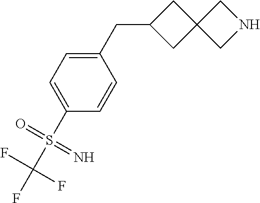 [SMILES: N=S(=O)(c1ccc(CC2CC3(CNC3)C2)cc1)C(F)(F)F] | 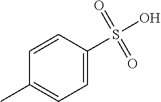 | [4-(2- azaspiro[3.3]heptan-6- ylmethyl)phenyl]- imino-oxo- (trifluoromethyl)-λ⁶- sulfane; 4- methylbenzenesulfonic acid | (4- bromophe- nyl)(imi- no)(trifluoro- methyl)-λ6- sulfanone (CAS: 1421133- 03-0) | 319.0 [M − TsOH + H]⁺ |
| D.3 | 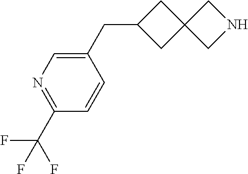 [SMILES: FC(F)(F)c1ccc(CC2CC3(CNC3)C2)cn1] | 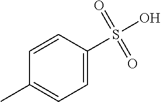 | 6-[[6-(trifluoromethyl)- 3-pyridyl]methyl]-2- azaspiro[3.3]heptan; 4- methylbenzenesulfonic acid | 5-bromo- 2- (trifluoro- methyl)py- ridine (CAS: 436799- 32-5) | 257.0 [M − TsOH + H]⁺ |
| D.4 | 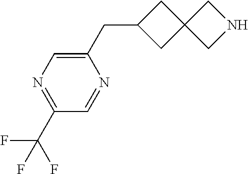 [SMILES: FC(F)(F)c1cnc(CC2CC3(CNC3)C2)cn1] | 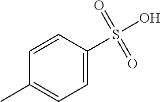 | 6-[[5- (trifluoromethyl)pyrazin- 2-yl]methyl]-2- azaspiro[3.3]heptane; 4- methylbenzenesulfonic acid | 2-bromo- 5- (trifluoro- methyl)py- razine (CAS: 799557- 87-2) | 258.2 [M − TsOH + H]⁺ |
| D.7 | 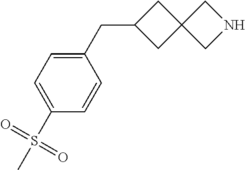 [SMILES: CS(=O)(=O)c1ccc(CC2CC3(CNC3)C2)cc1] | 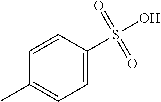 | 6-[(4- methylsulfonylphenyl) methyl]-2- azaspiro[3.3]heptane; 4- methylbenzenesulfonic acid | 4- bromo- phenyl methyl sulfone (CAS: 3466-32- 8) | 266.0 [M − TsOH + H]⁺ |
| D.8 | 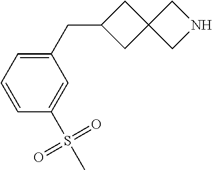 [SMILES: CS(=O)(=O)c1cccc(CC2CC3(CNC3)C2)c1] | 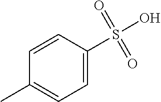 | 6-[(3- methylsulfonylphenyl) methyl]-2- azaspiro[3.3]heptane; 4- methylbenzenesulfonic acid | 3- bromophe- nylmeth- ylsulfone (CAS: 34896- 80-5) | 265.9 [M − TsOH + H]⁺ |
| D.9 | 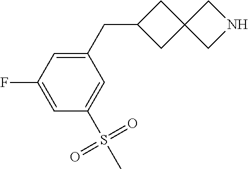 [SMILES: CS(=O)(=O)c1cc(F)cc(CC2CC3(CNC3)C2)c1] | 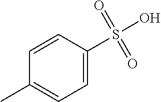 | 6-[(3-fluoro-5- methylsulfonyl- phenyl)methyl]-2- azaspiro[3.3]heptane; 4- methylbenzenesulfonic acid | 1-bromo- 3-fluoro- 5- methylsul- fonyl- benzene (CAS: 1207970- 78-2) | 284.0 [M − TsOH + H]⁺ |
| D.10 | 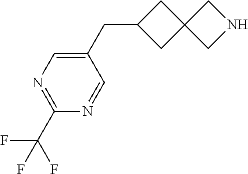 [SMILES: FC(F)(F)c1ncc(CC2CC3(CNC3)C2)cn1] | 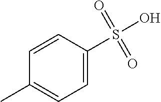 | 6-[[2- (trifluoromethyl)pyrimi- din-5-yl]methyl]-2- azaspiro[3.3]heptane; 4- methylbenzenesulfonic acid | 5-bromo- 2- (trifluoro- methyl)pyrimi- dine (CAS: 799557- 86-1) | 258.1 [M − TsOH + H]⁺ |
| D.11 | 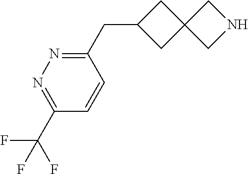 [SMILES: FC(F)(F)c1ccc(CC2CC3(CNC3)C2)nn1] | 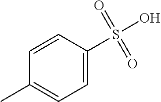 | 6-[[6- (trifluoromethyl)pyrida- zin-3-yl]methyl]-2- azaspiro[3.3]heptane; 4- methylbenzenesulfonic acid | 3-bromo- 6- (trifluoro- methyl)py- ridazine (CAS: 174607- 37-5) | 258.4 [M − TsOH + H]⁺ |
| D.12 | 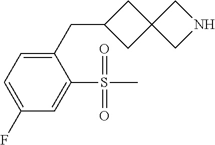 [SMILES: CS(=O)(=O)c1cc(F)ccc1CC1CC2(CNC2)C1] | 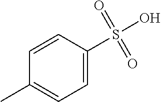 | 6-[(4-fluoro-2- methylsulfonyl- phenyl)methyl]-2- azaspiro[3.3]heptane; 4- methylbenzenesulfonic acid | 1-bromo- 4-fluoro- 2- methane- sulfonyl- benzene (CAS: 628692- 10-4) | 284.4 [M − TsOH + H]⁺ |
| D.13 | 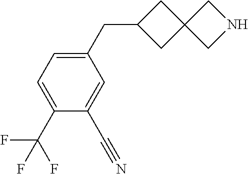 [SMILES: N#Cc1cc(CC2CC3(CNC3)C2)ccc1C(F)(F)F] | 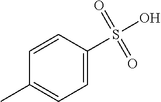 | 5-(2- azaspiro[3.3]heptan-6- ylmethyl)-2- (trifluoromethyl)benzo- nitrile; 4- methylbenzenesulfonic acid | 5-bromo- 2- (trifluoro- methyl) benzonitrile (CAS: 1208076- 28-1) | 281.1 [M − TsOH + H]⁺ |
| D.15 | 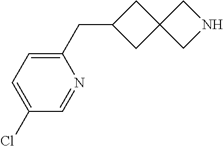 [SMILES: Clc1ccc(CC2CC3(CNC3)C2)nc1] | 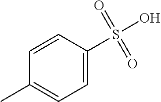 | 6-[(5-chloro-- pyridyl)methyl]-2- azaspiro[3.3]heptane; 4- methylbenzenesulfonic acid | 2-bromo- 5- chloro- pyridine (CAS: 40473- 01-06) | 223.2 [M − TsOH + H]⁺ |
| | *PtOH catalyst used in Step c) | | | | |
| B.14 | 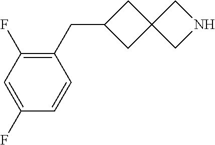 [SMILES: Fc1ccc(CC2CC3(CNC3)C2)c(F)c1] | 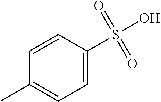 | 6-[(2,4- diflurophenyl)methyl]- 2-azaspiro[3.3]heptane; 4- methylbenzenesulfonic acid | 1-bromo- 2,4- difluoro- benzene (CAS: 348-57- 2) | 224.0 [M − TsOH + H]⁺ |
| D.22 | 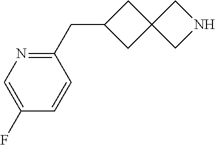 [SMILES: Fc1ccc(CC2CC3(CNC3)C2)nc1] | 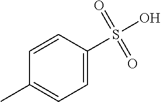 | 6-[(5-fluoro-2- pyridyl)methyl]-2- azaspiro[3.3]heptane; 4- methylbenzenesulfonic acid | 2-bromo- 5- fluoro- pyridine (CAS: 41404- 58-4) | 207.5 [M − TsOH + H]⁺ |
| D.24 | 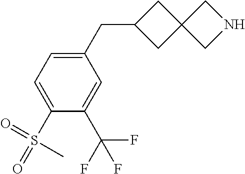 [SMILES: CS(=O)(=O)c1ccc(CC2CC3(CNC3)C2)cc1C(F)(F)F] | 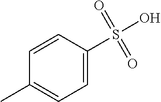 | 6-[[4-methylsulfonyl-3- (trifluoromethyl)phenyl] methyl]-2- azaspiro[3.3]heptane; 4- methylbenzenesulfonic acid | 4-bromo- 1- methyl- sulfonyl-2- (trifluoro- methyl) benzene (CAS: 300356- 32-5) | 334.2 [M − TsOH + H]⁺ |
| D.26 | 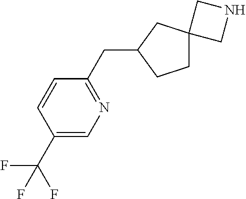 [SMILES: FC(F)(F)c1ccc(CC2CCC3(CNC3)C2)nc1] | 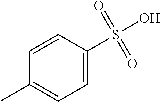 | 6-[[5-(trifluoromethyl)- 2-pyridyl)methyl]-2- azaspiro[3.4]octane; 4- methylbenzenesulfonic acid | 2-bromo- 5- (trifluoro- methyl) pyridine (CAS: 50488- 42-1) | 271.2 [M − TsOH + H]⁺ |
| D.47 | 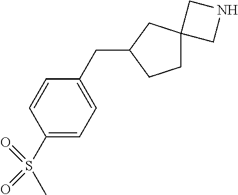 [SMILES: CS(=O)(=O)c1ccc(CC2CCC3(CNC3)C2)cc1] | 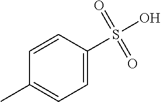 | 6-[(4- methylsulfonylphenyl) methyl]-2- azaspiro[3.4]octane; 4- methylbenzenesulfonic acid | 4- bromo- phenyl- methyl- sulfone (CAS: 3466-32- 8) | 280.0 [M − TsOH + H]⁺ |
| D.69 | 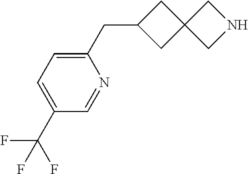 [SMILES: FC(F)(F)c1ccc(CC2CC3(CNC3)C2)nc1] | 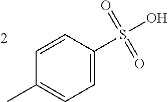 | 6-[[5-(trifluoromethyl)- 2-pyridyl]methyl]-2- azaspiro[3.3]heptane; di 4- methylbenzenesulfonic acid | 2-bromo- 5- (trifluoro- methyl) pyridine (CAS: 50488- 42-1) | 257.4 [M − 2TsOH + H]⁺ |
| D.72 | 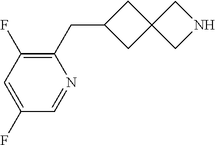 [SMILES: Fc1cnc(CC2CC3(CNC3)C2)c(F)c1] | 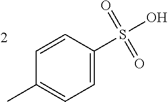 | 6-[(3,5-difluoro-2- pyridyl)methyl]-2- azaspiro[3.3]heptane; di 4- methylbenzenesulfonic acid | 2-bromo- 3,5- difluoro- pyridine (CAS: 660425- 16-1) | 225.0 [M − 2TsOH + H]⁺ |
| D.74 | 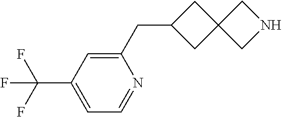 [SMILES: FC(F)(F)c1ccnc(CC2CC3(CNC3)C2)c1] | 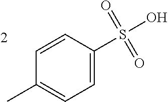 | 6-[[4-(trifluoromethyl)- 2-pyridyl]methyl]-2- azaspiro[3.3]heptane; di 4- methylbenzenesulfonic acid; | 2-bromo- 4- (trifluoro- methyl) pyridine (CAS: 175205- 81-9) | 257.5 [M − 2TsOH + H]⁺ |
| D.75 | 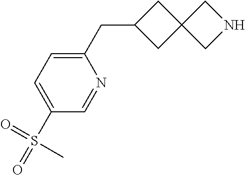 [SMILES: CS(=O)(=O)c1ccc(CC2CC3(CNC3)C2)nc1] | 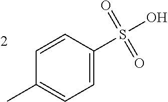 | 6-[(5-methylsulfonyl-2- pyridyl)methyl]-2- azaspiro[3.3]heptane; di 4- methylbenzenesulfonic acid | 2-bromo- 5- (methyl- sulfonyl)py- ridine (CAS: 343262- 51-1) | 267.2 [M − 2TsOH + H]⁺ |
| D.76 | 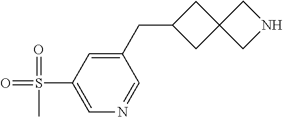 [SMILES: CS(=O)(=O)c1cncc(CC2CC3(CNC3)C2)c1] | 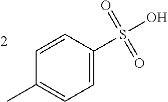 | 6-[(5-methysullfonyl-3- pyridyl)methyl]-2- azaspiro[3.3]heptane; 4- methylbenzenesulfonic acid | 3-bromo- 5- (methyl- sulfonyl) pyridine (CAS: 445491- 71-4) | 267.2 [M − 2TsOH + H]⁺ |
| D.78 | 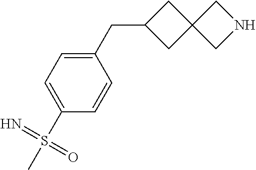 [SMILES: CS(=N)(=O)c1ccc(CC2CC3(CNC3)C2)cc1] | 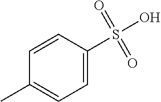 | [4-(2- azaspiro[3.3]heptan-6- ylmethyl)phenyl]- imino-methyl-oxo-λ⁶- sulfane; 4- methylbenzenesulfonic acid | 1-bromo- 4-(S- methyl- sulfonimi- doyl)benzene (CAS: 145026- 07-9) | 265.2 [M − TsOH + H]⁺ |
| D.79 | 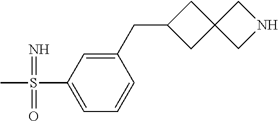 [SMILES: CS(=N)(=O)c1cccc(CC2CC3(CNC3)C2)c1] | 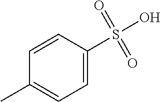 | [3-(2- azaspiro[3.3]heptan-6- ylmethyl)phenyl]- imino-methyl-oxo-λ⁶- sulfane; 4- methylbenzenesulfonic acid | (3- bromo- phenyl)(imi- no)(meth- yl)-λ6- sulfanone (CAS: 1789700- 09-9) | 265.2 [M − TsOH + H]⁺ |
| D.80a | 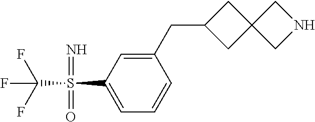 | 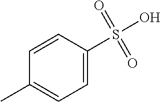 | [3-(2- azaspiro[3.3]heptan-6- ylmethyl)phenyl]- imino-oxo- (trifluoromethyl)-λ⁶- sulfane; 4- methylbenzenesulfonic acid | (3- bromo- phenyl)(imi- no)(trifluoro- methyl)- lambda6- sulfanone (CAS: 2411289- 54-6) | 319.0 [M − TsOH + H]⁺ |
| | (chiral separation after Step a, | | | | |
| | arbitrary assignment of | | | | |
| | stereochemistry) | | | | |
| D.80b | 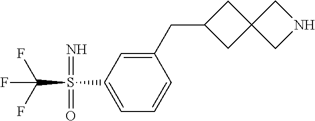 | 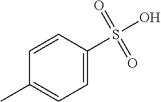 | [3-(2- azaspiro[3.3]heptan-6- ylmethyl)phenyl]- imino-oxo- (trifluoromethyl)-λ⁶- sulfane; 4- methylbenzenesulfonic acid | (3- bromo- phenyl)(imi- no)(trifluoro- methyl)- lambda6- sulfanone (CAS: 2411289- 54-6) | 319.0 [M − TsOH + H]⁺ |
| | (chiral separation after Step a, | | | | |
| | arbitrary assignment of | | | | |
| | stereochemistry) | | | | |
| D.82 | 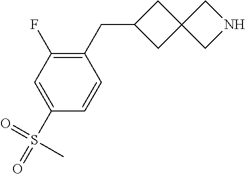 [SMILES: CS(=O)(=O)c1ccc(CC2CC3(CNC3)C2)c(F)c1] | 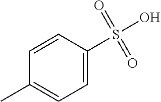 | 6-[(2-fluoro-4- methylsulfonyl- phenyl)methyl]-2- azaspiro[3.3]heptane; 4- methylbenzenesulfonic acid | 1-bromo- 2-fluoro- 4- methyl- sulfonyl- benzene (CAS: 1032825- 02-7) | 284.1 [M − TsOH + H]⁺ |
| D.87 | 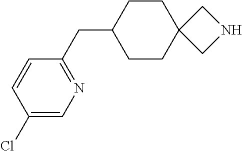 [SMILES: Clc1ccc(CC2CCC3(CC2)CNC3)nc1] | 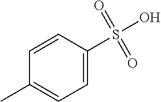 | 7-[(5-chloro-2- pyridyl)methyl]-2- azaspiro[3.5]nonane; 4- methylbenzenesulfonic acid | 2-bromo- 5- chloropy- ridine (CAS: 40473- 01-6) | 251.2 [M − TsOH + H]⁺ |
| | *Rh(10% on C) catalyst used in | | | | |
| | Step c) | | | | |
| D.90 | 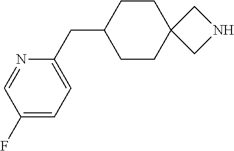 [SMILES: Fc1ccc(CC2CCC3(CC2)CNC3)nc1] | 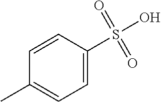 | 7-[(5-fluoro-2- pyridyl)methyl]-2- azaspiro[3.5]nonane; 4- methylbenzenesulfonic acid | 2-bromo- 5- fluoropyri- dine (CAS: 41404- 58-4) | 235.4 [M − TsOH + H]⁺ |
| D.92 | 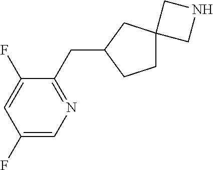 [SMILES: Fc1cnc(CC2CCC3(CNC3)C2)c(F)c1] | 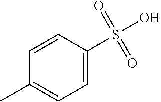 | 6-[(3,5-difluoro-2- pyridyl)methyl]-2- azaspiro[3.4]octane; 4- methylbenzenesulfonic acid | 2-bromo- 3,5-difluoro- pyridine (CAS: 660425- 16-1) | 239.2 [M − TsOH + H]⁺ |
| D.95 | 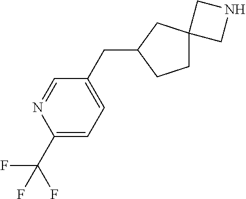 [SMILES: FC(F)(F)c1ccc(CC2CCC3(CNC3)C2)cn1] | 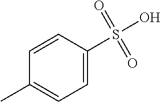 | 6-[[6-(trifluoromethyl)- 3-pyridyl]methyl]-2- azaspiro[3.4]octane; 4- methylbenzenesulfonic acid | 5-bromo- 2 - (trifluoro- methyl) pyridine (CAS: 436799- 32-5) | 271.1 [M − TsOH + H]⁺ |
| D.302 | 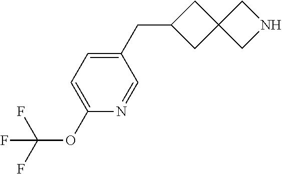 [SMILES: FC(F)(F)Oc1ccc(CC2CC3(CNC3)C2)cn1] | 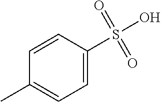 | 6-[[6- (trifluoromethoxy)-3- pyridyl]methyl]-2- azaspiro[3.3]heptane; 4- methylbenzenesulfonic acid | 5-bromo- 2- (trifluoro- methoxy) pyridine (CAS: 886371- 77-3) | 273.3 [M − TsOH + H]⁺ |
| D.305 | 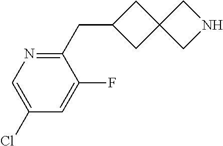 [SMILES: Fc1cc(Cl)cnc1CC1CC2(CNC2)C1] | 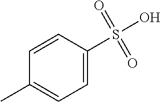 | 6-[(5-chloro-3-fluoro-2- pyridyl)methyl]-2- azaspiro[3.3]heptane; di 4- methylbenzenesulfonic acid | 2-bromo- 5-chloro- 3-fluoro- pyridine (CAS: 514797- 97-8) | 241.1 [M − 2TsOH + H]⁺ |
| D.306 | 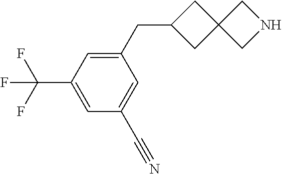 [SMILES: N#Cc1cc(CC2CC3(CNC3)C2)cc(C(F)(F)F)c1] | 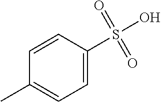 | 3-(2- azaspiro[3.3]heptan-6- ylmethyl)-5- (trifluoromethyl)benzo- nitrile; 4- methylbenzenesulfonic acid | 3-bromo- 5- (trifluoro- methyl)benzo- nitrile (CAS: 691877- 03-9) | 281.2 [M − TsOH + H]⁺ |
| D.307 | 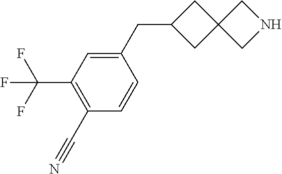 [SMILES: N#Cc1ccc(CC2CC3(CNC3)C2)cc1C(F)(F)F] | 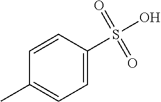 | 4-(2- azaspiro[3.3]heptan-6- ylmethyl)-2- (trifluoromethyl)benzo- nitrile; di 4- methylbenzenesulfonic acid | 4-bromo- 2- (trifluoro- methyl) benzonitrile (CAS: 191165- 13-6) | 281.1 [M − 2TsOH + H]⁺ |

### Example D.97

### 5-[[(6S)-2-azaspiro[3.4]octan-6-yl]oxy]-2-(trifluoromethyl)pyridine-4-carbonitrile; 4-methylbenzenesulfonic acid

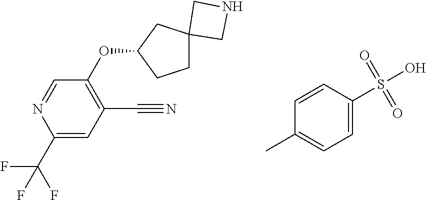 [SMILES: N#Cc1cc(C(F)(F)F)ncc1O[C@H]1CCC2(CNC2)C1 ; Cc1ccc(S(=O)(=O)O)cc1]

To a solution of (6S)-6-[[4-cyano-6-(trifluoromethyl)-3-pyridyl]oxy]-2-azaspiro[3.4]octane-2-carboxylic acid tert-butyl ester (0.310 g, 0.780 mmol) in isopropyl acetate (7 mL) was added p-toluenesulfonic acid monohydrate (222.58 mg, 1.17 mmol). The mixture was stirred at 80° C. for 5 h, before being evaporated. Trituration with Et₂O gave the title compound (366 mg, 94.94%) as a white solid. MS (ESI): m/z=298.2 [M+H]⁺

### Step a): (6S)-6-[[4-cyano-6-(trifluoromethyl)-3-pyridyl]oxy]-2-azaspiro[3.4]octane-2-carboxylic acid tert-butyl ester

To an ice-cold solution of rac-(6S)-6-hydroxy-2-azaspiro[3.4]octane-2-carboxylic acid tert-butyl ester (CAS RN: CAS: 2376903-72-7; 300 mg, 1.32 mmol) in dimethyl sulfoxide (0.80 mL) was added potassium tert-butylate (177.72 mg, 1.58 mmol) and 5-bromo-2-(trifluoromethyl)isonicotinonitrile (CAS RN: 1070892-04-4; 331.28 mg, 1.32 mmol). The mixture was stirred for 15 min at 0° C., before being diluted with EtOAc. The mixture was washed with diluted HCl, water, and brine. The organic layer was dried over Na₂SO₄, filtered, and evaporated. Purification by FC (SiO₂; heptane/EtOAc) gave the title compound (334 mg, 63.68% yield) as a white solid. MS (ESI): m/z=342.2 [M+H-Buten]⁺

In analogy to Example D.97, the following building blocks were generated using the relevant building blocks described in step a).

| | | | | | MS, |
|---|---|---|---|---|---|
| | | | | Building | ESI: |
| Ex. | Structure | | Systematic Name | Blocks | m/z |
| D.269 | 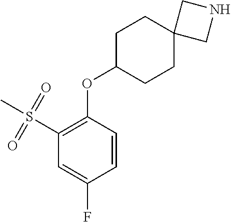 [SMILES: CS(=O)(=O)c1cc(F)ccc1OC1CCC2(CC1)CNC2] | 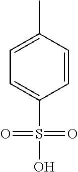 | 7-(4-fluoro-2- methylsulfonyl- phenoxy)-2- azaspiro[3.5]nonane; 4- methylbenzenesulfonic acid | 1,4-difluoro-2- mesyl-benzene (CAS RN: 61655-69-4), 7-hydroxy-2- azaspiro[3.5] nonane-2- carboxylic acid tert-butyl ester (CAS RN: 1363383-18-9) | 314.1 [M + H]⁺ |

### Example D.30

### N-(2-azaspiro[3.3]heptan-6-yl)-3-(trifluoromethyl)benzenesulfonamide; trifluoroacetic acid

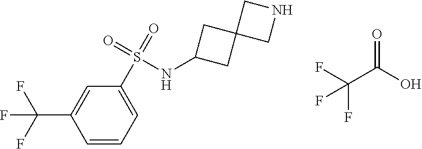 [SMILES: O=S(=O)(NC1CC2(CNC2)C1)c1cccc(C(F)(F)F)c1 ; O=C(O)C(F)(F)F]

To a solution of 6-[[3-(trifluoromethyl)phenyl]sulfonylamino]-2-azaspiro[3.3]heptane-2-carboxylic acid tert-butyl ester (1245 mg, 2.96 mmol) in dichloromethane (8 mL) was added TFA (3.38 g, 2.28 mL, 29.6 mmol) and the reaction mixture was then stirred at room temp for 18 h. Volatiles were removed in vacuo to yield 1910 mg of the crude title compound, purity of roughly 65% and major contaminant excess of TFA, which was used without further purification. MS (ESI): m/z=321.1 [M-TFA+H]⁺

### Step a) 6-[[3-(trifluoromethyl)phenyl]sulfonylamino]-2-azaspiro[3.3]heptane-2-carboxylic acid tert-butyl ester

To a solution of 6-amino-2-azaspiro[3.3]heptane-2-carboxylic acid tert-butyl ester (750 mg, 3.53 mmol) in dichloromethane (15 mL) cooled down to 0° C. was added DIPEA (685 mg, 926 μL, 5.3 mmol) and 3-(trifluoromethyl)benzenesulfonyl chloride (907 mg, 3.71 mmol) after which the reaction mixture was stirred at 0° C. for 30 min and at r.t for 1 h. The reaction mixture was poured into a separating funnel containing dichloromethane and aq. sol. Na₂CO₃ 1M. The organic phase was collected and the aqueous phase was back-extracted with dichloromethane. The combined organic phases were dried over sodium sulfate and evaporated down to dryness. The crude material was purified by flash chromatography with an eluent mixture of heptane and ethyl acetate (10% to 90%) to yield 775 mg of the title compound. MS (ESI): m/z=365.1 [M−tBu+H]⁺

In analogy to Example D.30, the following building blocks were generated using the relevant sulfonyl chloride building block. In Example D.175, an ethoxycarbamate protecting group was used instead of Boc, and was removed by alkaline hydrolysis. In Examples D.176-D.178, 4-(aminomethyl)piperidine-1-carboxylic acid tert-butyl ester was used in place of 6-amino-2-azaspiro[3.3]heptane-2-carboxylic acid tert-butyl ester. In some cases, alternative salts (e.g. trifluoroacetate, tosylate, ditosylate, hydrochloride) were also used. For D.178, the free base was isolated after deprotection with HCl and extraction from aqueous 33% NaOH solution in the final step.

| | | | | MS, |
|---|---|---|---|---|
| | | | Building | ESI: |
| Ex. | Structure | Systematic Name | Blocks | m/z |
| D.175 | 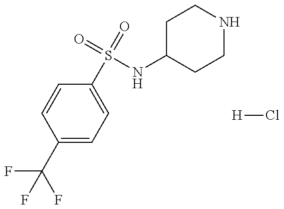 [SMILES: O=S(=O)(NC1CCNCC1)c1ccc(C(F)(F)F)cc1 ; Cl] | N-(4-piperidyl)-4- (trifluoromethyl)benzene- sulfonamide; hydrochloride | 4- (trifluoro- methyl) benzene- sulfonyl chloride (CAS: 2991-42- 6) | 309.1 [M − HCl + H]⁺ |
| D.176 | 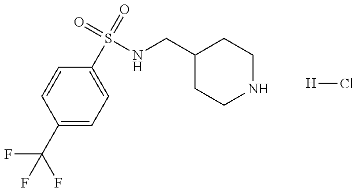 [SMILES: O=S(=O)(NCC1CCNCC1)c1ccc(C(F)(F)F)cc1 ; Cl] | N-(4-piperidylmethyl)- 4- (trifluoromethyl)benzene- sulfonamide; hydrochloride | 4- (trifluoro- methyl) benzene- sulfonyl chloride (CAS: 2991-42- 6) | 323.1 [M − HCl + H]⁺ |
| D.177 | 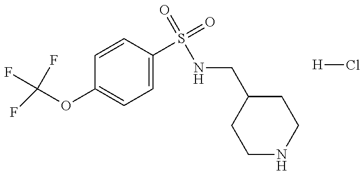 [SMILES: O=S(=O)(NCC1CCNCC1)c1ccc(OC(F)(F)F)cc1 ; Cl] | N-(4-piperidylmethyl)- 4- (trifluoromethoxy)ben- zenesulfonamide; hydrochloride | 4- (trifluoro- methoxy) benzene- sulfonyl chloride (CAS: 94108- 56-2) | 339.1 [M − HCl + H]⁺ |
| D.178 | 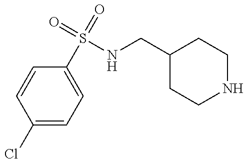 [SMILES: O=S(=O)(NCC1CCNCC1)c1ccc(Cl)cc1] | 4-chloro-N-(4- piperidylmethyl)benzene- sulfonamide | 4- chloro- benzene- sulfonyl chloride (CAS: 98-60-2) | 288.8 [M + H]⁺ |

### Example D.31

### N-[6-(trifluoromethyl)pyridazin-3-yl]-2-azaspiro13.31heptan-6-amine; trifluoroacetic acid

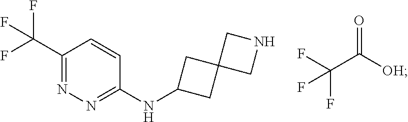

To a solution of 6-[[6-(trifluoromethyl)pyridazin-3-yl]amino]-2-azaspiro[3.3]heptane-2-carboxylic acid tert-butyl ester (708 mg, 1.94 mmol) in dichloromethane (8 mL) was added TFA (2.21 g, 1.49 mL, 19.4 mmol) and the reaction mixture was stirred at r.t for 18 h. Volatiles were removed in vacuo to yield 1310 mg of the crude title compound, purity roughly 55% with major contaminant excess TFA, which was used without further purification. MS (ESI): m/z=259.1 [M-TFA+H]⁺

### Step a) 6-[[6-(trifluoromethyl)pyridazin-3-yl]amino]-2-azaspiro[3.3]heptane-2-carboxylic acid tert-butyl ester

To a solution of 6-amino-2-azaspiro[3.3]heptane-2-carboxylic acid tert-butyl ester (832 mg, 3.92 mmol) in N,N-dimethylformamide (12 mL) was added DIPEA (690 mg, 932 μL, 5.34 mmol) and 3-chloro-6-(trifluoromethyl)pyridazine (650 mg, 3.56 mmol) after which the reaction mixture was stirred at 80° C. for 18 h. Volatiles were removed in vacuo and the crude residue was partitioned between ethyl acetate and sat. aq. NH₄Cl solution. The organic phase was collected and the aqueous phase was back-extracted with ethyl acetate. The combined organic phases were dried over sodium sulfate and evaporated down to dryness. The crude material was purified by flash chromatography, using an eluent mixture of dichloromethane and methanol (0% to 10%) to yield 708 mg of the title compound. MS (ESI): m/z=359.2 [M+H]⁺

### Example D.54

### 2-[[4-(trifluoromethylsulfonyl)phenyl]methyl]-2,6-diazaspiro[3.3]heptane; 4-methylbenzenesulfonic acid

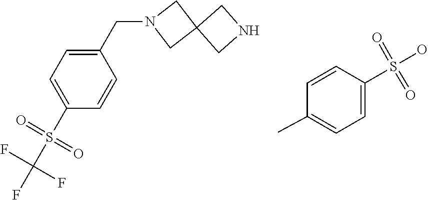

p-Toluenesulfonic acid monohydrate (3.98 g, 20.93 mmol) was added to a stirred solution of tert-butyl 6-[[4-(trifluoromethylsulfonyl)phenyl]methyl]-2,6-diazaspiro[3.3]heptane-2-carboxylate (4.0 g, 9.51 mmol) in EtOAc (70 mL). The reaction mixture was stirred at 60° C. for 48 h. The precipitate was collected by filtration and washed twice with MTBE (2*50 mL), to give the title compound (6047.3 mg, 90.84% yield) as a white solid. MS (ESI): m/z=321.2 [M+H]⁺

### Step a): tert-butyl 6-[[4-(trifluoromethylsulfonyl)phenyl]methyl]-2,6-diazaspiro[3.3]heptane-2-carboxylate

A solution of 4-(trifluoromethylsulfonyl)benzaldehyde (5.48 g, 23.01 mmol) and tert-butyl 2,6-diazaspiro[3.3]heptane-2-carboxylate hydrochloride (3.6 g, 15.34 mmol) in DCE (100 mL) was treated with triethylamine (2.35 mL, 16.87 mmol) and stirred for 10 min at 23° C. The mixture was treated with acetic acid (1.84 g, 30.67 mmol), and the mixture was heated to 60° C. and stirred for 60 min at this temperature, before being cooled down. Sodium triacetoxyborohydride (5.85 g, 27.61 mmol) was added, and the mixture was stirred for 18 h at 23° C., before being treated with saturated aqueous NaHCO₃. The mixture was extracted with DCM (2×100 mL), and the combined organic layers were washed with brine, dried over Na₂SO₄, filtered, and evaporated. Purification by FC (SiO₂; PE/MTBE) gave the title compound (4.3 g, 63.35% yield) as a light yellow solid. MS (ESI): m/z=421.2 [M+H]⁺

In analogy to Example D.54, the following building blocks were generated using the relevant building blocks described in step a.

| | | | | | MS, |
|---|---|---|---|---|---|
| | | | | Bulding | ESI: |
| Ex. | Structure | | Systematic Name | Blocks | m/z |
| D.99 | 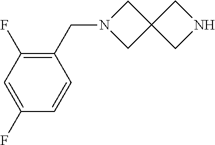 [SMILES: Fc1ccc(CN2CC3(CNC3)C2)c(F)c1] | 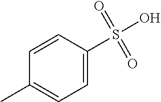 | 2-[(2,4- difluorophenyl)meth- yl]-2,6- diazaspiro[3.3]heptane; 4- methylbenzenesulfo- nic acid | tert-Butyl 2,6- diazaspiro[3.3] heptane- 2- carboxylate oxalate (2:1) (CAS: 1041026- | 225.1 [M − TsOH + H]⁺ |
| | | | | 71-4); 2,4- | |
| | | | | difluoroben- | |
| | | | | zaldehyde | |
| | | | | (CAS: | |
| | | | | 1550-35-2) | |
| D.100 | 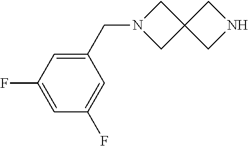 [SMILES: Fc1cc(F)cc(CN2CC3(CNC3)C2)c1] | 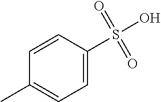 | 2-[(3,5- difluorophenyl)meth- yl]-2,6- diazaspiro[3.3]heptane; 4- methylbenzenesulfo- nic acid | tert-Butyl 2,6- diazaspiro[3.3] heptane- 2- carboxylate oxalate(2:1) (CAS: 1041026- 71-4); 3,5- difluoroben- zaldehyde (CAS: 32085-88-4) | 225.0 [M − TsOH + H]⁺ |
| D.104 | 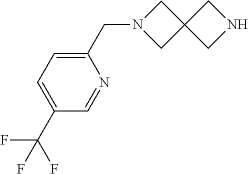 [SMILES: FC(F)(F)c1ccc(CN2CC3(CNC3)C2)nc1] | 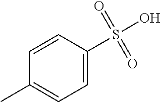 | 2-[[5- (trifluoromethyl)-2- pyridyl]methyl]-2,6- diazaspiro[3.3]heptane; 4- methylbenzenesulfo- nic aicd | tert-Butyl 2,6- diazaspiro[3.3] heptane- 2- carboxylate oxalate(2:1) (CAS: 1041026- 71-4); 5- (trifluorometh- | 258.4 [M − TsOH + H]⁺ |
| | | | | yl)-2- | |
| | | | | pyridinecar- | |
| | | | | boxyaldehyde | |
| | | | | (CAS: | |
| | | | | 31224-82-5) | |
| D.106 | 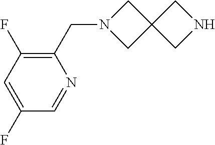 [SMILES: Fc1cnc(CN2CC3(CNC3)C2)c(F)c1] | 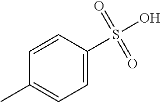 | 2-[(3,5-difluoro-2- pyridyl)methyl]-2,6- diazaspiro[3.3]heptane; 4- methylbenzenesulfo- nic acid | tert-Butyl 2,6- diazaspiro[3.3] heptane- 2- carboxylate oxalate(2:1) (CAS: 1041026- | 226.2 [M − TsOH + H]⁺ |
| | | | | 71-4); 3,5- | |
| | | | | difluoropyri- | |
| | | | | dine-2- | |
| | | | | carbaldehyde | |
| | | | | (CAS: | |
| | | | | 780801-58- | |
| | | | | 3) | |
| D.107 | 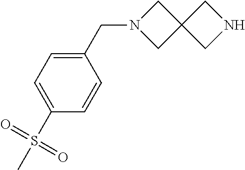 [SMILES: CS(=O)(=O)c1ccc(CN2CC3(CNC3)C2)cc1] | 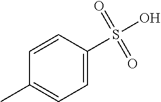 | 2-[(4- methylsulfonylphenyl) methyl]-2,6- diazaspiro[3.3]heptane; di 4- methylbenzenesulfo- nic acid | tert-Butyl 2,6- diazaspiro[3.3] heptane- 2- carboxylate oxalate(2:1) (CAS: 1041026- 71-4); 4- mesylbenzal- | 267.1 [M − 2TsOH + H]⁺ |
| | | | | dehyde | |
| | | | | (CAS: | |
| | | | | 5398-77-6) | |
| D.108 | 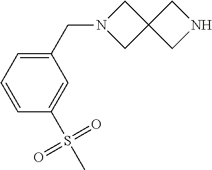 [SMILES: CS(=O)(=O)c1cccc(CN2CC3(CNC3)C2)c1] | 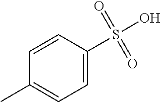 | 2-[(3- methylsulfonylphenyl) methyl]-2,6- diazaspiro[3.3]heptane; di 4- methylbenzenesulfo- nic acid | tert-Butyl 2,6- diazaspiro[3.3] heptane- 2- carboxylate oxalate(2:1) (CAS: 1041026- 71-4); 3- mesylbenzal- | 267.1 [M − 2TsOH + H]⁺ |
| | | | | dehyde | |
| | | | | (CAS: 43114- | |
| | | | | 43-8) | |
| D.109 | 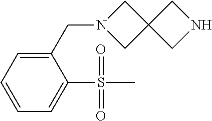 [SMILES: CS(=O)(=O)c1ccccc1CN1CC2(CNC2)C1] | 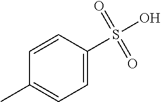 | 2-[(2- methylsulfonylphenyl) methyl]-2,6- diazaspiro[3.3]heptane; di 4- methylbenzenesulfo- nic acid | tert-Butyl 2,6- diazaspiro[3.3] heptane- 2- carboxylate- oxalate(2:1) (CAS: | 267.1 [M − 2TsOH + H]⁺ |
| | | | | 1041026- | |
| | | | | 71-4); 2- | |
| | | | | mesylbenzal- | |
| | | | | dehyde | |
| | | | | (CAS: | |
| | | | | 5395-89-1) | |
| D.116 | 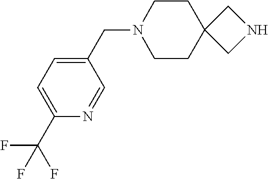 [SMILES: FC(F)(F)c1ccc(CN2CCC3(CC2)CNC3)cn1] | 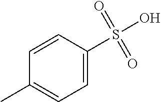 | 7-[[6- (trifluoromethyl)-3- pyridyl]methyl]-2,7- diazaspiro[3.5]nonane; 4- methylbenzenesulfo- nic acid | 2,7- diazaspiro[3.5] nonane- 2- carboxylic acid tert- butyl ester (CAS: 236406-55- 6); 6- (trifluorometh- yl)nicotin- aldehdye (CAS: 386704-12- 7) | 286.1 [M − TsOH + H]⁺ |
| D.118 | 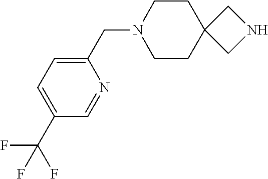 [SMILES: FC(F)(F)c1ccc(CN2CCC3(CC2)CNC3)nc1] | 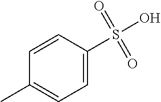 | 7-[[5- (trifluoromethyl)-2- pyridyl]methyl]-2,7- diazaspiro[3.5]nonane; 4- methylbenzenesulfo- nic acid | 2,7- diazaspiro[3.5] nonane- 2- carboxylic acid tert- butyl ester (CAS: 236406-55- 6); 5- (trifluorometh- | 286.1 [M − TsOH + H]⁺ |
| | | | | yl)picolin- | |
| | | | | aldehyde | |
| | | | | (CAS: | |
| | | | | 31224-82-5) | |
| D.119 | 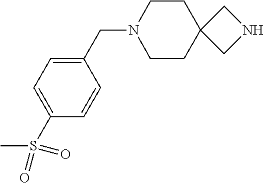 [SMILES: CS(=O)(=O)c1ccc(CN2CCC3(CC2)CNC3)cc1] | 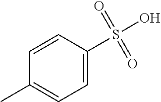 | 7-[(4- methylsulfonylphenyl) methyl]-2,7- diazaspiro[3.5]nonane; di 4- methylbenzenesulfo- nic acid | 2,7- diazaspiro[3.5] nonane- 2- carboxylic acid tert- butyl ester (CAS: 236406-55- 6); 4- mesylbenzal- | 295.2 [M − 2TsOH + H]⁺ |
| | | | | dehyde | |
| | | | | (CAS: | |
| | | | | 5398-77-6) | |
| D.121 | 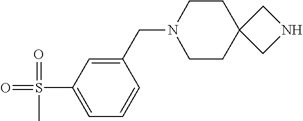 [SMILES: CS(=O)(=O)c1cccc(CN2CCC3(CC2)CNC3)c1] | 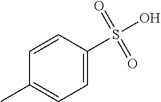 | 7-[(3- methylsulfonylphenyl) methyl]-2,7- diazaspiro[3.5]nonane; 4- methylbenzenesulfo- nic acid | 2,7- diazaspiro[3.5] nonane- 2- carboxylic acid tert- butyl ester (CAS: | 295.2 [M − 2TsOH + H]⁺ |
| | | | | 236406-55- | |
| | | | | 6); 3- | |
| | | | | mesylbenzal- | |
| | | | | dehyde | |
| | | | | (CAS: 431 | |
| | | | | 14-43-8) | |
| D.122 | 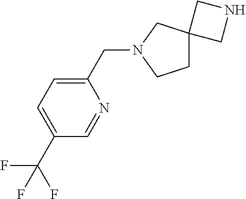 [SMILES: FC(F)(F)c1ccc(CN2CCC3(CNC3)C2)nc1] | 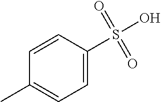 | 6-[[5- (trifluoromethyl)-2- pyridyl]methyl]-2,6- diazaspiro[3.4]octane; 4- methylbenzenesulfo- nic acid | tert-butyl 2,6- diazaspiro[3.4] octane-2- carboxylate (CAS: 885270-84- 8); 5- (trifluorometh- yl)picolin- aldehyde (CAS: | 272.0 [M − 2TsOH + H]⁺ |
| | | | | 31224-82-5) | |
| D.123 | 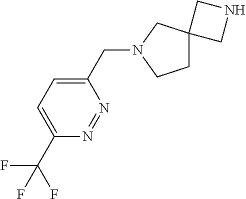 [SMILES: FC(F)(F)c1ccc(CN2CCC3(CNC3)C2)nn1] | 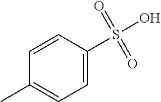 | 6-[[6- (trifluoromethyl)pyri- dazin-3-yl]methyl]- 2,6- diazaspiro[3.4]octane; 4- methylbenzenesulfo- nic acid | tert-butyl 2,6- diazaspiro[3.4] octane-2- carboxylate (CAS: 885270-84- 8); 6- (trifluorometh- yl)pyrida- zine-3- carbaldehyde (CAS: | 273.0 [M − 2TsOH + H]⁺ |
| | | | | 1245643- | |
| | | | | 49-5) | |
| D.124 | 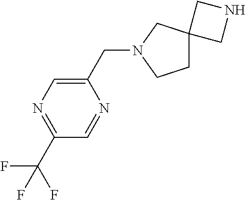 [SMILES: FC(F)(F)c1cnc(CN2CCC3(CNC3)C2)cn1] | 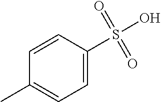 | 6-[[5- (trifluoromethyl)pyra- zin-2-yl]methyl]- 2,6- diazaspiro[3.4]octane; 4- methylbenzenesulfo- nic acid | tert-butyl 2,6- diazaspiro[3.4] octane-2- carboxylate (CAS: 885270-84- 8); 5- (trifluorometh- yl)pyrazine- 2- carbaldehyde (CAS: | 273.2 [M − 2TsOH + H]⁺ |
| | | | | 1196151- | |
| | | | | 36-6) | |
| D.130 | 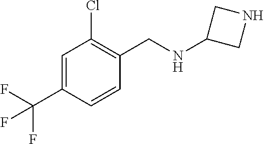 [SMILES: FC(F)(F)c1ccc(CNC2CNC2)c(Cl)c1] | 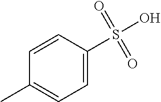 | N-[[2-chloro-4- (trifluoromethyl)phe- nyl]methyl]azetidin- 3-amine; 4- methylbenzenesulfo- nic acid | 1-BOC-3- (amino)azeti- dine (CAS: 193269-78- 2); 2- chloro-4- (trifluorometh- yl)benzalde- hyde | 265.1 [M − TsOH + H]⁺ |
| | | | | (CAS: | |
| | | | | 82096-91-1) | |
| D.132 | 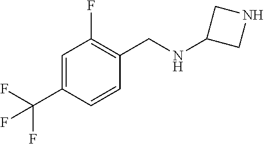 [SMILES: Fc1cc(C(F)(F)F)ccc1CNC1CNC1] | 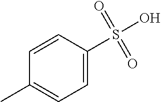 | N-[[2-fluoro-4- (trifluoromethyl)phe- nyl]methyl]azetidin- 3-amine; 4- methylbenzenesulfo- nic acid | 1-BOC-3- (amino)azeti- dine (CAS: 193269-78- 2); 2-fluoro- 4- (trifluorometh- yl)benzalde- hyde | 249.1 [M − TsOH + H]⁺ |
| | | | | (CAS: | |
| | | | | 89763-93-9) | |
| D.137 | 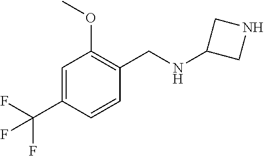 [SMILES: COc1cc(C(F)(F)F)ccc1CNC1CNC1] | 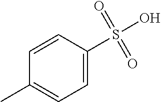 | N-[[2-methoxy-4- (trifluoromethyl)phe- nyl]methyl]azetidin- 3-amine; 4- methylbenzenesulfo- nic acid | 1-BOC-3- (amino)azeti- dine (CAS: 193269-78- 2); 2- methoxy-4- (trifluorometh- yl)benzalde- hyde (CAS: | 261.1 [M − TsOH + H]⁺ |
| | | | | 132927-09- | |
| | | | | 4) | |
| D.303 | 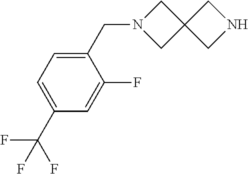 [SMILES: Fc1cc(C(F)(F)F)ccc1CN1CC2(CNC2)C1] | 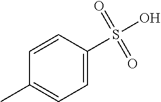 | 2-[[2-fluoro-4- (trifluoromethyl)phe- nyl]methyl]-2,6- diazaspiro[3.3]heptane; 4- methylbenzenesulfo- nic acid | tert-Butyl 2,6- diazaspiro[3.3] heptane- 2- carboxylate oxalate(2:1) (CAS: 1374659- 01-4); 2- fluoro-4- | 275.2 [M − TsOH + H]⁺ |
| | | | | (trifluorometh- | |
| | | | | yl)benzalde- | |
| | | | | hyde | |
| | | | | (CAS: | |
| | | | | 89763-93-9) | |

### Example D.55

### 2-[3-(trifluoromethoxy)phenyl]sulfonyl-2,6-diazaspiro[3.3]heptane; trifluoroacetic acid

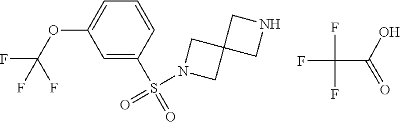

To a solution of 2-[3-(trifluoromethoxy)phenyl]sulfonyl-2,6-diazaspiro[3.3]heptane-6-carboxylic acid tert-butyl ester (1350 mg, 3.2 mmol) in dichloromethane (13.5 mL) was added TFA (3.64 g, 2.46 mL, 32.0 mmol) and the reaction mixture was stirred at room temperature for 18 h. Volatiles were removed in vacuo to yield 1855 mg of the crude title compound (purity roughly 70% major contaminant excess of TFA), which was used without further purification. MS (ESI): m/z=323.1 [M-TFA+H]⁺

### Step a) 2-[3-(trifluoromethoxy)phenyl]sulfonyl-2,6-diazaspiro[3.3]heptane-6-carboxylic acid tert-butyl ester

To a suspension of 2,6-diazaspiro[3.3]heptane-2-carboxylic acid tert-butyl ester (790 mg, 3.98 mmol) in dichloromethane (18 mL) cooled down to 0° C. was added DIPEA (1.04 mL, 5.98 mmol) and 3-(trifluoromethoxy)benzenesulfonyl chloride (1.04 g, 3.98 mmol) after which the reaction mixture was stirred at 0° C. for 10 min and at room temp. for 1 h. The reaction mixture was diluted with dichloromethane and extracted with aq. Na₂CO₃ 1M solution. The organic phase was collected and the aqueous phase was back-extracted with dichloromethane. The combined organic phases were dried over sodium sulfate and evaporated down to dryness. The residue was purified by FC (SiO₂; heptane/EtOAc) to yield 1350 mg of the title compound. MS (ESI): m/z=367.1 [M−tBu+H]⁺

In analogy to Example D.55, the following building blocks were generated using the relevant building blocks in Step a). For Examples D.167, 2,7-diazaspiro[3.5]nonane-2-carboxylic acid tert-butyl ester was used in place of 2,6-diazaspiro[3.3]heptane-2-carboxylic acid tert-butyl ester.

| | | | | MS, |
|---|---|---|---|---|
| | | | Building | ESI: |
| Ex. | Structure | Systematic Name | Blocks | m/z |
| D.58 | 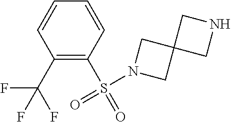 [SMILES: O=S(=O)(c1ccccc1C(F)(F)F)N1CC2(CNC2)C1] | 2-[2- (trifluoromethyl) phenyl]sulfonyl-2,6- diazaspiro[3.3] heptane; trifluoroacetic acid | 2- (trifluoromethyl) benzene- sulfonyl chloride (CAS: 776- 04-5) | 307.2 [M − TFA + H]⁺ |
| | 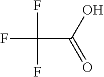 | | | |
| D.60 | 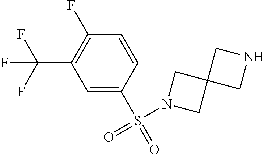 [SMILES: O=S(=O)(c1ccc(F)c(C(F)(F)F)c1)N1CC2(CNC2)C1] | 2-[4-fluoro-3- (trifluoromethyl) phenyl]sulfonyl-2,6- diazaspiro[3.3] heptane; 4- methylbenzene- sulfonic acid | 4-fluoro-3- (trifluoromethyl) benzene- sulfonyl chloride (CAS: 1682-10-6) | 325.0 [M − TsOH + H]⁺ |
| | 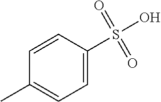 | | | |
| D.62 | 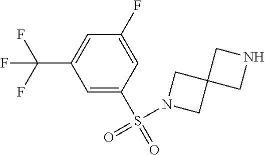 [SMILES: O=S(=O)(c1cc(F)cc(C(F)(F)F)c1)N1CC2(CNC2)C1] | 2-[3-fluoro-5- (trifluoromethyl) phenyl]sulfonyl-2,6- diazaspiro[3.3] heptane; 4- methylbenzene- sulfonic acid | 3-fluoro-5- (trifluoromethyl) benzene- sulfonyl chloride (CAS: 886499-99- 6) | 325.0 [M − TsOH + H]⁺ |
| | 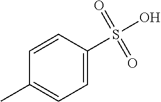 | | | |
| D.65 | 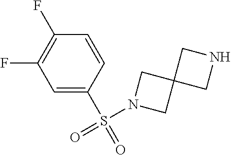 [SMILES: O=S(=O)(c1ccc(F)c(F)c1)N1CC2(CNC2)C1] | 2-(3,4- difluorophenyl) sulfonyl-2,6- diazaspiro[3.3] heptane; 4- methylbenzene- sulfonic acid | 3,4- difluoro- benzenesulfonyl chloride (CAS: 145758-05- 0) | 275.0 [M − TsOH + H]⁺ |
| | 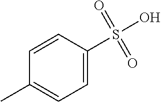 | | | |
| D.68 | 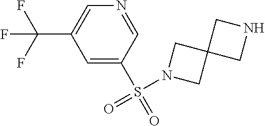 [SMILES: O=S(=O)(c1cncc(C(F)(F)F)c1)N1CC2(CNC2)C1] | 2-[[5- (trifluoromethyl)-3- pyridyl]sulfonyl]- 2,6-diazaspiro[3.3] heptane; 4- methylbenzene- sulfonic acid | 5- (trifluoromethyl) pyridine- 3-sulfonyl chloride (CAS: 1060802- 03-0) | 308.0 [M − TsOH + H]⁺ |
| | 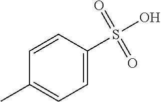 | | | |
| D.33 | 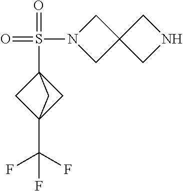 [SMILES: O=S(=O)(N1CC2(CNC2)C1)C12CC(C(F)(F)F)(C1)C2] | 2-[[3- (trifluoromethyl)-1- bicyclo[1.1.1] pentanyl] sulfonyl]-2,6- diazaspiro[3.3] heptane; trifluoroacetic acid | 3- (trifluoromethyl) bicyclo [1.1.1] pentane-1- sulfonyl chloride (CAS: 2167111- 82-0) | 297.1 [M − TFA + H]⁺ |
| | 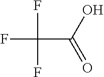 | | | |
| D.167 | 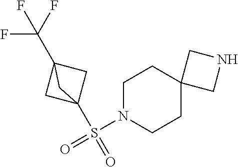 [SMILES: O=S(=O)(N1CCC2(CC1)CNC2)C12CC(C(F)(F)F)(C1)C2] | 7-[[3- (trifluoromethyl)-1- bicyclo[1.1.1]pentanyl] sulfonyl]-2,7- diazaspiro[3.5]nonane; trifluoroacetic acid | 3- (trifluoromethyl) bicyclo [1.1.1] pentane-1- sulfonyl chloride (CAS: 2167111- 82-0) | 325.1 [M − TFA + H]⁺ |
| | 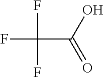 | | | |

### Example D.101

### 2-[[6-(trifluoromethyl)pyridazin-3-yl]methyl]-2,6-diazaspiro[3.3]heptane; di 4-methylbenzenesulfonic acid

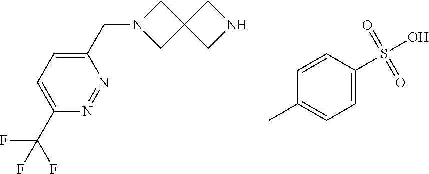 [SMILES: FC(F)(F)c1ccc(CN2CC3(CNC3)C2)nn1 ; Cc1ccc(S(=O)(=O)O)cc1]

A mixture of p-toluenesulfonic acid (1010 mg, 5.86 mmol), tert-butyl 6-[[6-(trifluoromethyl)pyridazin-3-yl]methyl]-2,6-diazaspiro[3.3]heptane-2-carboxylate (1000 mg, 2.79 mmol) in EtOAc (10 mL) was stirred at 80° C. for 12 h. The mixture was filtered and cake was concentrated to give the title compound (1450 mg, 86% yield). MS (ESI): m/z=259.2 [M-2TsOH+H]⁺

### Step a) 3-(chloromethyl)-6-(trifluoromethyl)pyridazine

A mixture of 3-methyl-6-(trifluoromethyl)pyridazine (2.0 g, 12.3 mmol) in 1,2-dichloroethane (40 mL) was added trichloroisocyanuric acid (958 mg, 4.12 mmol). The mixture was heated to 80° C. and stirred for 12 h. The residue was purified by FC to give the title compound (1.3 g, 54% yield) as a white solid. MS (ESI): m/z=197.1 [M+H]⁺

### Step b) tert-butyl 6-[[6-(trifluoromethyl)pyridazin-3-yl]methyl]-2,6-diazaspiro[3.3]heptane-2-carboxylate

To a solution of 3-(chloromethyl)-6-(trifluoromethyl)pyridazine (1.3 g, 6.61 mmol), tert-butyl 2,6-diazaspiro[3.3]heptane-2-carboxylate; oxalic acid (3.22 g, 6.61 mmol) in ACN (10 mL) was added K₂CO₃ (1.83 g, 13.2 mmol) at 25° C. The mixture was stirred at 25° C. for 12 h. The mixture was stirred at 50° C. for 2 h. The residue was purified by silica column (petroleum ether: ethyl acetate=10:1 to 0:1) and concentrated under reduced pressure to give the title compound (1.7 g, 71.7% yield) as a white solid. MS (ESI): m/z=359.3 [M+H]⁺

In analogy to Example D.101, the following building blocks were generated using the relevant commercial building blocks in Step b).

| | | | | MS, |
|---|---|---|---|---|
| | | | Building | ESI: |
| Ex. | Structure | Systematic Name | Blocks | m/z |
| D.105 | 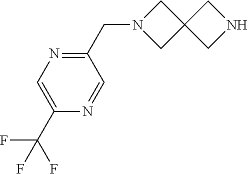 [SMILES: FC(F)(F)c1cnc(CN2CC3(CNC3)C2)cn1] | 2-[[5- (trifluoromethyl) pyrazin-2-yl]methyl]- 2,6- diazaspiro[3.3] heptane; 4- methylbenzenesulfonic acid | 2- (bromo- methyl)-5- (trifluoro- methyl)pyrazine (CAS: 1447914- 67-1) | 259.4 [M + H]⁺ |
| | 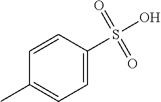 | | | |
| D.111 | 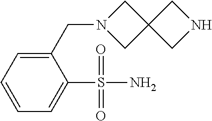 [SMILES: NS(=O)(=O)c1ccccc1CN1CC2(CNC2)C1] | 2-(2,6- diazaspiro[3.3] heptan-2- ylmethyl)benzene- sulfonamide; 4- methylbenzenesulfonic acid | 2- (chloromethyl) benzene- 1- sulfonamide (CAS: 81629-77-8) | 266.2 [M + H]⁺ |
| | 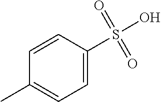 | | | |
| D.112 | 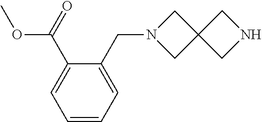 [SMILES: COC(=O)c1ccccc1CN1CC2(CNC2)C1] | methyl 2-(2,6- diazaspiro[3.3] heptan-2- ylmethyl)benzoate; 4- methylbenzenesulfonic acid | methyl 2- (bromo- methyl)benzoate (CAS: 2417-73-4) | 247.2 [M + H]⁺ |
| | 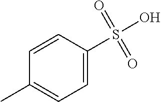 | | | |
| D.304 | 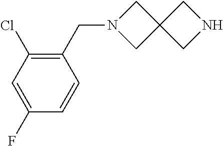 [SMILES: Fc1ccc(CN2CC3(CNC3)C2)c(Cl)c1] | 2-[(2-chloro-4- fluoro- phenyl)methyl]-2,6- diazaspiro[3.3] heptane; trifluoroacetic acid | 2-chloro-5- fluorobenzyl bromide (CAS: 45767-66-6) | 241.1 [M + H]⁺ |
| | 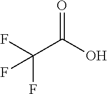 | | | |

### Example D.110

### 2-[[4-fluoro-2-(trifluoromethyl)phenyl]methyl]-2,6-diazaspiro[3.3]heptane; trifluoroacetic acid

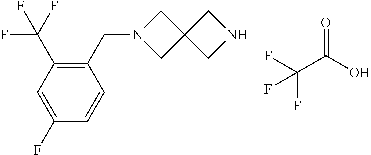

To a solution of tert-butyl 6-(4-fluoro-2-(trifluoromethyl)benzyl)-2,6-diazaspiro[3.3]heptane-2-carboxylate (455 mg, 1.09 mmol) in dichloromethane (4 mL) was added TFA (843 μL, 10.9 μL) and the reaction mixture was stirred at RT for 18 h. Volatiles were removed in vacuo to yield 685 mg of the crude title compound (purity roughly 80%) which was used without further purification. MS (ESI): m/z=275.2 [M-TFA+H]⁺

### Step a) tert-butyl 6-(4-fluoro-2-(trifluoromethyl)benzoyl)-2,6-diazaspiro[3.3]heptane-2-carboxylate

To a solution of tert-butyl 2,6-diazaspiro[3.3]heptane-2-carboxylate (400 mg, 2.02 mmol) in CH₂Cl₂ (9 mL) cooled down to 0° C. was added DIPEA (652 mg, 881 μL, 5.04 mmol) and 4-fluoro-2-(trifluoromethyl)benzoyl chloride (503 mg, 2.22 mmol). The reaction mixture was stirred at 0° C. for 10 min and at RT for 18 h. The reaction mixture was diluted with dichloromethane and extracted with aq. Na₂CO₃ 1M solution, the organic phase was collected and the aqueous phase was back-extracted with dichloromethane. The combined organic phases were dried over sodium sulfate and evaporated down to dryness. The crude material was purified by flash chromatography, using an eluent mixture of heptane and ethyl acetate (5% to 80%) to give the title compound (569 mg). MS (ESI): m/z=389.3 [M+H]⁺

### Step b) tert-butyl 6-(4-fluoro-2-(trifluoromethyl)benzyl)-2,6-diazaspiro[3.3]heptane-2-carboxylate

To a solution of tert-butyl 6-(4-fluoro-2-(trifluoromethyl)benzoyl)-2,6-diazaspiro[3.3]heptane-2-carboxylate (565 mg, 1.45 mmol) in dry THF (5 mL) was slowly added borane tetrahydrofuran complex 1.0 M (3.64 mL, 3.64 mmol) and the reaction mixture was then refluxed for 20 h. The reaction was cooled down to 0° C. followed by addition of slow addition of methanol to quench excess borane after which it was stirred at 23° C. for 15 min followed by stirring at 55° C. for 18 h. Volatiles were removed in vacuo and the crude residue was directly purified by flash chromatography using an eluent mixture of dichloromethane and methanol (0% to 10%) to yield 417 mg of the title compound. MS (ESI): m/z=375.2 [M+H]⁺

### Example D.149 N-(1-methylcyclopropyl)-2,6-diazaspiro[3.3]heptane-2-sulfonamide;2,2,2-trifluoroacetic acid

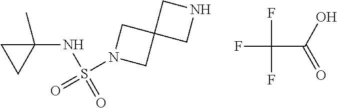

To a solution of 2-[(1-methylcyclopropyl)sulfamoyl]-2,6-diazaspiro[3.3]heptane-6-carboxylic acid tert-butyl ester (448 mg, 1.35 mmol) in dichloromethane (5 mL) was added TFA (1.54 g, 1.04 mL, 13.52 mmol) and the reaction mixture was stirred at room temperature for 18 h. Volatiles were removed in vacuo to yield the crude title compound (736 mg), roughly 63% purity with excess TFA as major contaminant, which was used directly without further purification. MS (ESI): m/z=232.2 [M-TFA+H]⁺

### Step a) 2-(2-methylimidazol-1-yl)sulfonyl-2,6-diazaspiro[3.3]heptane-6-carboxylic acid tert-butyl ester

To a solution of 2-methyl-1-(2-methylimidazol-1-yl)sulfonyl-imidazole (1.5 g, 6.63 mmol) in dichloromethane (27 mL) under an inert atmosphere cooled down to 0° C. was slowly added methyl trifluoromethanesulfonate (730 μL, 6.63 mmol). Upon completion of reagent addition a white precipitate began to form and the reaction mixture was allowed to stir at 0° C. and slowly warm up to room temperature overnight. Volatiles were removed in vacuo to give 2.60 g of the crude intermediate as a white solid which was used without further purification. The crude solid was dissolved in acetonitrile, extra dry (27 mL) followed by addition of 2,6-diazaspiro[3.3]heptane-2-carboxylic acid tert-butyl ester (1.31 g, 6.63 mmol) after which the reaction mixture was stirred at 80° C. for 64 h. The reaction mixture was diluted with ethyl acetate, poured into a separating funnel and extracted with aq. sol. Na₂CO₃ 1 M. The organic phase was collected and the aqueous phase was back-extracted with ethyl acetate. The combined organic phases were dried over sodium sulfate and evaporated down to dryness. The crude material was submitted for SFC purification to yield 1372 mg of the title compound. MS (ESI): m/z=343.2 [M-TFA+H]⁺

### Step b) 2-[(1-methylcyclopropyl)sulfamoyl]-2,6-diazaspiro[3.3]heptane-6-carboxylic acid tert-butyl ester

To a solution of 2-(2-methylimidazol-1-yl)sulfonyl-2,6-diazaspiro[3.3]heptane-6-carboxylic acid tert-butyl ester (778 mg, 2.27 mmol) in dichloromethane (10 mL) cooled down to 0° C. was added methyl trifluoromethanesulfonate (392 mg, 263 μL, 2.39 mmol) and the reaction mixture was stirred at 0° C. for 3 h. Volatiles were removed in vacuo and the crude white solid was re-dissolved in acetonitrile, extra dry (10 mL) followed by addition of (1-methylcyclopropyl)amine (242 mg, 3.41 mmol) after which the reaction mixture was stirred at 70° C. for 18 h. Volatiles were removed in vacuo. The crude residue was dissolved in ethyl acetate, transferred into a separating funnel and extracted with sat. aq. solution Na₂CO₃. The organic phase was collected and the aqueous phase was back-extracted with ethyl acetate. The combined organic phases were dried over sodium sulfate and evaporated down to dryness. Purification by FC (SiO₂; DCM/MeOH) gave the title compound (448 mg). MS (ESI): m/z=330.3 [M+H]⁺

In analogy to Example D.149, the following building blocks were generated using the relevant commercial building blocks in Step b).

| | | | | MS, |
|---|---|---|---|---|
| | | | Building | ESI: |
| Ex. | Structure | Systematic Name | Blocks | m/z |
| D.150 | 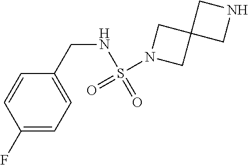 [SMILES: O=S(=O)(NCc1ccc(F)cc1)N1CC2(CNC2)C1] | N-[(4- fluorophenyl) methyl]-2,6- diazaspiro[3.3] heptane-2- sulfonamide; trifluoroacetic acid | (4- fluorobenzyl) amine (CAS: 140- 75-0) | 286.1 [M − TFA + H]⁺ |
| | 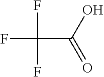 | | | |
| D.151 | 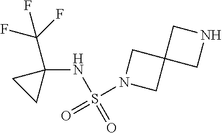 [SMILES: O=S(=O)(NC1(C(F)(F)F)CC1)N1CC2(CNC2)C1] | N-[1- (trifluoromethyl) cyclopropyl]-2,6- diazaspiro[3.3] heptane-2- sulfonamide; trifluoroacetic acid | [1- (trifluoro- methyl)cyclo- propyl]amine (CAS: 112738-68- 8) | 286.1 [M − TFA + H]⁺ |
| | 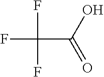 | | | |

### Example D.152

### N-[[1-(trifluoromethyl)cyclopropyl]methyl]-2,6-diazaspiro[3.3]heptane-2-sulfonamide; 4-methylbenzenesulfonic acid

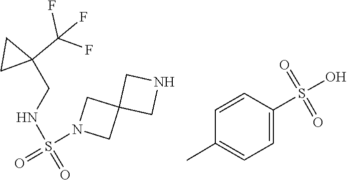 [SMILES: O=S(=O)(NCC1(C(F)(F)F)CC1)N1CC2(CNC2)C1 ; Cc1ccc(S(=O)(=O)O)cc1]

A solution of tert-butyl 2-[[1-(trifluoromethyl)cyclopropyl]methylsulfamoyl]-2,6-diazaspiro[3.3]heptane-6-carboxylate (450 mg, 1.13 mmol) and p-toluenesulfonic acid monohydrate (429 mg, 2.25 mmol) in EtOAc (15 mL) was heated at reflux for 2 h, then cooled to RT and stirred for another 16 h. The precipitate was collected by filtration, washed with EtOAc (5 mL) and dried under vacuum to provide the title compound (298 mg, 53% yield). MS (ESI): m/z=300.2 [M-TsOH+H]⁺

### Step a) tert-butyl 2-chlorosulfonyl-2,6-diazaspiro[3.3]heptane-6-carboxylate

To a stirred solution of sulfuryl chloride (0.63 g, 4.69 mmol) in DCM (15 mL) at 0° C. was added a mixture of triethylamine (1.19 mL, 8.52 mmol) and tert-butyl 2,6-diazaspiro[3.3]heptane-2-carboxylate hydrochloride (1.0 g, 4.26 mmol) (as a solution in 15 mL of DCM) at such a rate as to keep the temperature below 20° C. The reaction mixture was stirred at room temperature for 18 h, then evaporated to dryness. The crude sulfamoyl chloride (30% purity) was used directly in next step without further purification.

### Step b) tert-butyl 2-[[ ]-(trifluoromethyl)cyclopropyl]methylsulfamoyl]-2,6-diazaspiro[3.3]heptane-6-carboxylate

To a stirred mixture of tert-butyl 2-chlorosulfonyl-2,6-diazaspiro[3.3]heptane-6-carboxylate (310 mg, 1.04 mmol) and [1-(trifluoromethyl)cyclopropyl]methanamine; hydrochloride (238 mg, 1.36 mmol) in ACN (10 mL), N,N-diisopropylethylamine (0.55 mL, 3.13 mmol) was added. Then the tube was 1 sealed and stirred at 40° C. for 18 h. Then the reaction mixture was concentrated to dryness and the residue was taken up in DCM (20 mL) and the organics washed with water (2×5 mL) and saturated brine solution (5 mL). The organic layer was dried (Na₂SO₄) before being concentrated to dryness in vacuo. The (290 mg, 66% yield) was used to the next step without further purification. MS (ESI): m/z=398.2 [M−H]⁻

### Example D.157

### N-(2-azaspiro[3.3]heptan-6-yl)-1-(trifluoromethyl)cyclopropanecarboxamide; 4-methylbenzenesulfonic acid

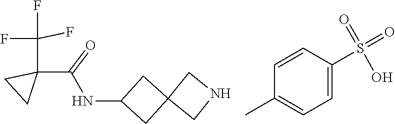

A solution of tert-butyl 6-[[1-(trifluoromethyl)cyclopropanecarbonyl]amino]-2-azaspiro[3.3]heptane-2-carboxylate (729 mg, 1.73 mmol, 67% yield) and p-toluenesulfonic acid monohydrate (0.98 g, 5.17 mmol) in EtOAc (50 mL) was heated at reflux for 2 h, then cooled to RT and stirred for another 16 h.. The precipitate was collected by filtration, washed with ethyl acetate (20 mL) and dried under vacuum to provide N-(2-azaspiro[3.3]heptan-6-yl)-1-(trifluoromethyl)cyclopropanecarboxamide; 4-methylbenzenesulfonic acid (729 mg, 67% yield) as a white solid. MS (ESI): m/z=249.1 [M-TsOH+H]⁺

### Step a) tert-butyl 6-[[ ]-(trifluoromethyl)cyclopropanecarbonyl]amino]-2-azaspiro[3.3]heptane-2-carboxylate

To a stirred solution of 1-(trifluoromethyl)cyclopropane-1-carboxylic acid (0.5 g, 3.22 mmol), tert-butyl 6-amino-2-azaspiro[3.3]heptane-2-carboxylate; hydrochloride (0.8 g, 3.22 mmol) and HATU (1.47 g, 3.86 mmol) in DMF (8 mL), N,N-diisopropylethylamine (2.24 mL, 12.9 mmol) was added in one portion at RT. The resulting mixture was stirred overnight (18 h) at RT. Then poured onto water (50 mL), and resulting precipitate was filtered, washed with water and dried to give the title compound (0.900 g, 76% yield) as a yellow solid. MS (ESI): m/z=347.2 [M+H]⁺

### Example D.179

### 5-(azetidin-3-yl)-N-[[1-(trifluoromethyl)cyclopropyl]methyl]pyridin-2-amine;4-methylbenzenesulfonic acid

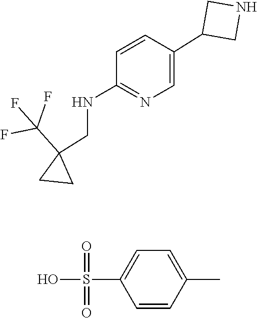 [SMILES: FC(F)(F)C1(CNc2ccc(C3CNC3)cn2)CC1 ; Cc1ccc(S(=O)(=O)O)cc1]

To a stirred solution of tert-butyl 3-[6-[[1-(trifluoromethyl)cyclopropyl]methylamino]-3-pyridyl]azetidine-1-carboxylate (5.5 g, 14.8 mmol) in EtOAc (300 mL), p-toluenesulfonic acid monohydrate (7.04 g, 37.0 mmol) was added. Then RM was stirred at 50° C. for 24 h. The reaction mixture was evaporated in vacuo and obtained residue was stirred with TBME (300 mL) for 12 h. The obtained precipitate was filtered, washed with TBME (2×200 mL) and dried to give the title compound (5.32 g, 55% yield) as a light yellow solid. MS (ESI): m/z=272.2 [M+H]⁺

### Step a) tert-butyl 3-(6-bromo-3-pyridyl)azetidine-]-carboxylate

The stirred mixture of tert-butyl 3-(p-tolylsulfonylhydrazono)azetidine-1-carboxylate (CAS: 1510865-66-3) (68.0 g, 200 mmol), 2-bromopyridine-5-boronic acid (53.8 g, 266 mmol) and potassium carbonate (41.5 g, 301 mmol) in dry 1,4-dioxane (2800 mL) were refluxed for 24 h (Argon). Then obtained precipitate was filtered off and filtrate was evaporated to dryness. The obtained residue was partioned between TBME (2000 mL) and water (500 mL). The organic layer was washed with brine (100 mL), dried over Na₂SO₄ and evaporated in vacuum. The crude product was purified by flash chromatography to obtain the title compound (13.8 g, 21% yield) as a light yellow oil. MS (ESI): m/z=313.0 [M+H]⁺

### Step b) tert-butyl 3-[6-[[ ]-(trifluoromethyl)cyclopropyl]methylamino]-3-pyridyl]azetidine-]-carboxylate

The mixture of tert-butyl 3-(6-bromo-3-pyridyl)azetidine-1-carboxylate (7.0 g, 22.4 mmol), [1-(trifluoromethyl)cyclopropyl]methanamine; hydrochloride (5.89 g, 33.5 mmol), tris(dibenzylideneacetone)dipalladium (1.02 g, 1.12 mmol), XantPhos (1.03 g, 1.79 mmol) and sodium tert-butoxide (6.44 g, 67.1 mmol) was sealed and stirred in degassed Toluene (100 mL) at 100° C. for 24 h (Argon atmosphere). Then RM was cooled to RT and filtered through a pad of SiO₂, washed with toluene (300 mL) and concentrated in vacuo. Purification by FC (SiO₂; hexane/MTBE) gave the title compound (5.5 g, 63% yield) as orange crystals. MS (ESI): m/z=372.2 [M+H]⁺

In analogy to Example D.179, the following building blocks were generated using the relevant commercial building blocks.

| | | | | MS, |
|---|---|---|---|---|
| | | | Building | ESI: |
| Ex. | Structure | Systematic Name | Blocks | m/z |
| D.183 | 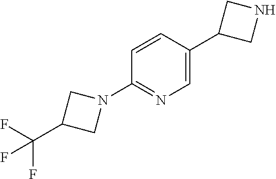 [SMILES: FC(F)(F)C1CN(c2ccc(C3CNC3)cn2)C1] | 5-(azetidin-3-yl)-2- [3- (trifluoromethyl) azetidin-1-yl]pyridine; di 4- methylbenzenesulfonic acid | 3- (trifluoro- methyl) azetidine; hydrochloride (CAS: 1221272- 90-7) | 258.2 [M − 2TsOH + H]⁺ |
| | 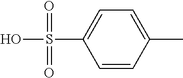 | | | |
| | * Xphos catalyst used in Step b) | | | |

### Example D.180

### 5-(azetidin-3-yl)-N-[[1-(trifluoromethyl)cyclopropyl]methyl]pyrazin-2-amine; di 4-methylbenzenesulfonic acid

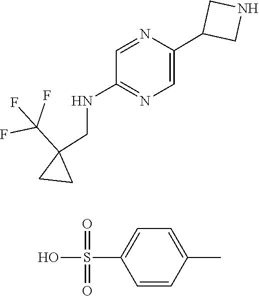

A mixture of tert-butyl 3-[5-[[1-(trifluoromethyl)cyclopropyl]methylamino]pyrazin-2-yl]azetidine-1-carboxylate (1.9 g, 5.1 mmol) and p-toluenesulfonic acid (1.14 g, 6.63 mmol) in EtOAc (20 mL) was stirred at 80° C. for 12 h. A further addition of p-toluenesulfonic acid (87.9 mg, 0.510 mmol) was made, and the mixture was stirred for another 12 h at 80° C. The reaction was concentrated under vacuum to give a residue. To the residue was added 80 mL water and the mixture was lyophilized to the title compound (2.38 g, 75% yield) as a yellow solid. MS (ESI): m/z=273.2 [M-2TsOH+H]⁺

### Step a) tert-butyl 3-(5-bromopyrazin-2-yl)azetidine-1-carboxylate

To a mixture of zinc (4131 mg, 63.2 mmol) in THF (96 mL) was added 1,2-dibromoethane (791 mg, 4.21 mmol) and CHLOROTRIMETHYLSILANE (458 mg, 4.21 mmol). The mixture was heated to 60° C. and stirred for 15 min. Then a mixture of 1-BOC-3-iodoazetidine (12.5 g, 44.2 mmol) in DMA (96 mL) was added. The mixture was stirred for another 15 min. The mixture was cooled to 20° C. and 2-bromo-5-iodopyrazine (12.0 g, 42.1 mmol), 1,1-BIS(DIPHENYLPHOSPHINO)FERROCENE-PALLADIUM(II)DICHLORIDEDICHLOROMETHANECOMPLEX (1720 mg, 2.11 mmol) and COPPER(I) IODIDE (0.07 mL, 2.11 mmol) were added. The mixture was heated to 80° C. and stirred for 12 h. The mixture was added to 200 mL water and extracted with EtOAc (200 mL×3). The combined organic phases were evaporated and purified by FC (SiO₂; PE/EtOAc), to give the title compound (4.9 g, 37% yield) as a white solid. MS (ESI): m/z=258.1 [M-C₄H₈+H]⁺

### Step b) tert-butyl 3-[5-[[ ]-(trifluoromethyl)cyclopropyl]methylamino]pyrazin-2-yl]azetidine-1-carboxylate

To a solution of [1-(trifluoromethyl)cyclopropyl]methanamine; hydrochloride (1667 mg, 9.49 mmol), tert-butyl 3-(5-bromopyrazin-2-yl)azetidine-1-carboxylate (3000 mg, 9.55 mmol) and sPhos-Pd-G3 (836 mg, 0.950 mmol) in t-Amyl-OH (55.5 mL) was added tBuONa 1M THF SOLUTION (14.3 mL, 28.6 mmol) under N₂ atmosphere, the mixture was degassed with N₂ for 1 min and stirred under N₂ atmosphere at 100° C. for 12 h. The mixture was evaporated and purified by RP-HPLC, to give the title compound (1.9 g, 53% yield) as a yellow solid. MS (ESI): m/z=373.1 [M+H]⁺

In analogy to Example D.180, the following building blocks were generated using the relevant commercial building blocks. In some cases alternative acids (e.g. TFA, HCl were used for the final deprotection).

| | | | | MS, |
|---|---|---|---|---|
| | | | Building | ESI: |
| Ex. | Structure | Systematic Name | Blocks | m/z |
| D.181 | 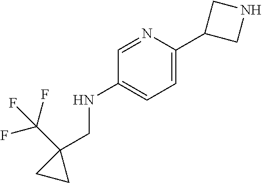 [SMILES: FC(F)(F)C1(CNc2ccc(C3CNC3)nc2)CC1] | 6-(azetidin-3-yl)-N- [[1- (trifluoromethyl) cyclopropyl]methyl] pyridin-3-amine; di 4- methylbenzenesulfonic acid | 2,5- dibromo- pyridine (CAS: 624-28-2); [1- (trifluoro- methyl)cyclo- propyl] methanamine hydrochloride (CAS: 1783418-59-6) | 272.2 [M − 2TsOH + H]⁺ |
| | 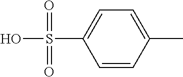 | | | |
| | * tBuXPhos-Pd-G3 catalyst used | | | |
| | in Step b) | | | |
| D.190 | 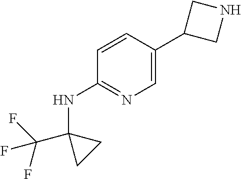 [SMILES: FC(F)(F)C1(Nc2ccc(C3CNC3)cn2)CC1] | 5-(azetidin-3-yl)-N- [1- (trifluoromethyl) cyclopropyl]pyridin-2- amine; 4- methylbenzenesulfonic acid | Step b) 5- (1-Boc-3- azetidinyl)- 2- chloropyridine (CAS: 870689-19- 3); 1- (trifluoromethyl) cyclopro- panamine | 258.2 [M − TsOH + H]⁺ |
| | 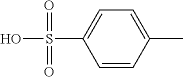 | | hydrochloride (CAS: 112738-67- 7) | |
| | * tBuXPhos-Pd-G3 catalyst used | | | |
| | in Step b) | | | |

### Example D.185

### 5-(azetidin-3-yl)-2-[3-(trifluoromethyl)azetidin-1-yl]pyrimidine; di 4-methylbenzenesulfonic acid

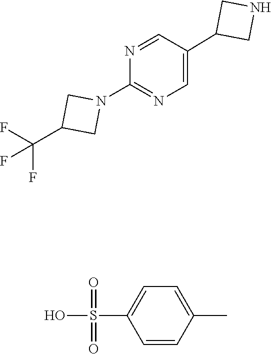 [SMILES: FC(F)(F)C1CN(c2ncc(C3CNC3)cn2)C1 ; Cc1ccc(S(=O)(=O)O)cc1]

A mixture of p-toluenesulfonic acid (1474 mg, 8.56 mmol), tert-butyl 3-[2-[3-(trifluoromethyl)azetidin-1-yl]pyrimidin-5-yl]azetidine-1-carboxylate (2.36 g, 6.59 mmol) in EtOAc (20 mL) was stirred at 80° C. for 12 h. The mixture was filtered and cake was concentrated to give the title compound (3.49 g, 88% yield) as a white solid. MS (ESI): m/z=259.2 [M-2TsOH+H]⁺

### Step a) 5-bromo-2-[3-(trifluoromethyl)azetidin-1-yl]pyrimidine

A solution of DIPEA (4.8 g, 37.1 mmol), 3-(trifluoromethyl)azetidine; hydrochloride (2.0 g, 12.4 mmol) and 5-bromo-2-fluoropyrimidine (2.63 g, 14.9 mmol) in DMSO (15 mL) was stirred at 100° C. for 16 h.

The aqueous phase was extracted with ethyl acetate (100 mL×3).The combined organic phase was washed with brine (100 mL×3), dried with anhydrous Na₂SO₄, filtered and concentrated in vacuum. Purification by FC (SiO₂; PE/EtOAc) gave the title compound (3.24 g, 93% yield) as a yellow solid. ¹H NMR (400 MHz, CHLOROFORM-d) δ=8.35 (s, 2H), 4.33-4.25 (m, 2H), 4.23-4.16 (m, 2H), 3.46-3.31 ppm (m, 1H).

### Step b) tert-butyl 3-[2-[3-(trifluoromethyl)azetidin-1-yl]pyrimidin-5-yl]azetidine-]-carboxylate

To a 250 mL vial equipped with a stir bar was added tert-butyl 3-bromoazetidine-1-carboxylate (3482 mg, 14.8 mmol), 5-bromo-2-[3-(trifluoromethyl)azetidin-1-yl]pyrimidine (3200 mg, 11.4 mmol), Ir[dF(CF₃)ppy]₂(dtbbpy)PF₆ (127 mg, 0.110 mmol), NiCl₂·dtbbpy (22.6 mg, 0.060 mmol), Na₂CO₃ (2405 mg, 22.7 mmol), TTMSS (2822 mg, 11.4 mmol) in DME (100 mL). The vial was sealed and placed under nitrogen. The reaction was stirred and irradiated with a 34 W blue LED lamp (7 cm away), with cooling fan to keep the reaction temperature at 25° C. for 14 h. The mixture was filtered and evaporated. Purification by RP-HPLC gave the title compound (2.4 g, 59% yield) as a white solid. MS (ESI): m/z=359.3 [M+H]⁺

In analogy to Example D.180, the following building blocks were generated using the relevant commercial building blocks. In some cases, alternative conditions for the S_{N}Ar reaction in Step a) were used such as K₂CO₃, DMF, 110° C. microwave.

| | | | | MS, |
|---|---|---|---|---|
| | | | Building | ESI: |
| Ex. | Structure | Systematic Name | Blocks | m/z |
| D.187 | 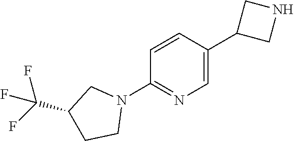 [SMILES: FC(F)(F)[C@H]1CCN(c2ccc(C3CNC3)cn2)C1] | 5-(azetidin-3-yl)-2- [(3S)-3- (trifluoromethyl) pyrrolidin-1- yl]pyridine;4- methylbenzenesulfonic acid | 5-bromo-2- fluoropyridine (CAS: 624-28-2); 3- (trifluoro- methyl) pyrrolidine hydrochloride | 272.1 [M − 2TsOH + H]⁺ |
| | 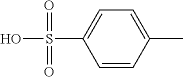 | | (CAS: 1189485- 03-7) | |
| | *arbitrary assignment of | | | |
| | stereochemistry, separated by | | | |
| | chiral SFC after Step b) | | | |
| D.188 | 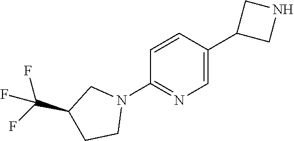 [SMILES: FC(F)(F)[C@@H]1CCN(c2ccc(C3CNC3)cn2)C1] | 5-(azetidin-3-yl)-2- [(3R)-3- (trifluoromethyl) pyrrolidin-1- yl]pyridine;4- methylbenzenesulfonic acid | 5-bromo-2- fluoropyridine (CAS: 624-28-2); 3- (trifluoro- methyl) pyrrolidine hydrochloride | 272.1 [M − 2TsOH + H]⁺ |
| | 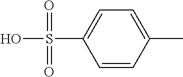 | | (CAS: 1189485- 03-7) | |
| | *arbitrary assignment of | | | |
| | stereochemistry, separated by | | | |
| | chiral SFC after Step b) | | | |
| D.189 | 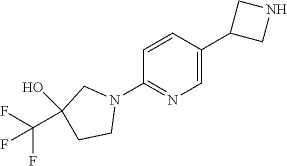 [SMILES: OC1(C(F)(F)F)CCN(c2ccc(C3CNC3)cn2)C1] | 1-[5-(azetidin-3-yl)- 2-pyridyl]-3- (trifluoromethyl) pyrrolidin-3-ol; 4- methylbenzenesulfonic acid | 5-bromo-2- fluoropyridine (CAS: 624-28-2); 3- (trifluoro- methyl) pyrrolidin-3-ol hydrochloride (CAS: 1334147-81-7) | 288.1 [M − 2TsOH + H]⁺ |
| | 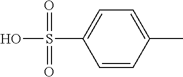 | | | |
| D.196 | 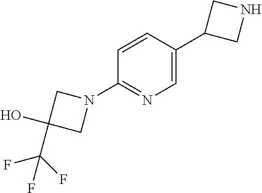 [SMILES: OC1(C(F)(F)F)CN(c2ccc(C3CNC3)cn2)C1] | 1-[5-(azetidin-3-yl)- 2-pyridyl]-3- (trifluoromethyl) azetidin-3-ol; di 4- methylbenzenesulfonic acid | 5-bromo-2- fluoropyridine (CAS: 624-28-2); 3- (trifluoro- methyl) azetidin-3-ol hydrochloride (CAS: 848192-96-1) | 274.2 [M − 2TsOH + H]+ |
| | 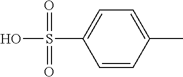 | | | |

### Example D.225

### 3-[[2-fluoro-4-(trifluoromethylsulfonyl)phenyl]methoxy]azetidine;4-methylbenzenesulfonic acid

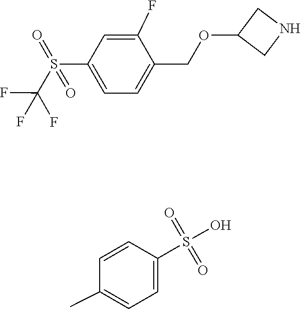 [SMILES: O=S(=O)(c1ccc(COC2CNC2)c(F)c1)C(F)(F)F ; Cc1ccc(S(=O)(=O)O)cc1]

A solution of p-toluenesulfonic acid (674.84 mg, 3.92 mmol) and tert-butyl 3-[[2-fluoro-4-(trifluoromethylsulfonyl)phenyl]methoxy]azetidine-1-carboxylate (1.35 g, 3.27 mmol) in EtOAc (14 mL) was stirred at 80° C. for 12 h. The mixture was filtered and the cake was dried, to give the title compound (915 mg, 56% yield) as a white solid. MS (ESI): m/z=314.1 [M-TsOH+H]⁺

### Step a): tert-butyl 3-[(2-fluoro-4-iodo-phenyl)methoxy]azetidine-1-carboxylate

To a solution of tert-butyl 3-hydroxyazetidine-1-carboxylate (CAS RN: 141699-55-0; 2.92 g, 16.83 mmol) in THF (50 mL) were added potassium tert-butoxide (3.78 g, 33.66 mmol) and 1-(bromomethyl)-2-fluoro-4-iodo-benzene (CAS RN: 85510-81-2; 5.3 g, 16.83 mmol), at 25° C. under Ar. The mixture was stirred for 12 h at 30° C., before being evaporated. Purification by FC (SiO₂; PE/EtOAc) and RP-HPLC gave the title compound (3.0 g, 44% yield) as a colorless oil. 1H NMR (400 MHz, CDCl₃) δ=7.51 (dd, J=1.4, 8.1 Hz, 1H), 7.43 (dd, J=1.6, 9.1 Hz, 1H), 7.14 (t, J=7.8 Hz, 1H), 4.46 (s, 2H), 4.32 (tdd, J=2.0, 4.3, 6.4 Hz, 1H), 4.11-4.05 (m, 2H), 3.89-3.83 (m, 2H), 1.44 ppm (s, 9H).

### Step b): tert-butyl 3-[[2-fluoro-4-(trifluoromethylsulfanyl)phenyl]methoxy]azetidine-]-carboxylate

Two batches were set up in parallel. To a 40 mL vial equipped with a magnetic stir bar were added trifluoromethylsulfanylsilver (769.63 mg, 3.68 mmol), tert-butyl 3-[(2-fluoro-4-iodo-phenyl)methoxy]azetidine-1-carboxylate (1.0 g, 2.46 mmol) and bpy (383.53 mg, 2.46 mmol) in ACN (10 mL), and then was added CuI (467.68 mg, 2.46 mmol) under N₂ atmosphere. The mixture stirred at 100° C. for 17 h under N₂ atmosphere. The mixture was filtered and evaporated. Purification by FC (SiO₂; PE/EtOAc) gave the title compound (1.68 g, 90% yield) as a white solid. MS (ESI): m/z=282.2 [M-C₅H₈O₂+H]⁺

### Step c): tert-butyl 3-[[2-fluoro-4-(trifluoromethylsulfonyl)phenyl]methoxy]azetidine-1-carboxylate

To a solution of tert-butyl 3-[[2-fluoro-4-(trifluoromethylsulfanyl)phenyl]methoxy]azetidine-1-carboxylate (1.58 g, 4.14 mmol) in 1:1:2 1,2-dichloroethane/ACN/water (60 mL) was added sodium periodate (1.77 g, 8.29 mmol) and ruthenium(III) chloride hydrate (9.34 mg, 0.040 mmol), at 0° C. The mixture was stirred for 12 h at 30° C., before being extracted with EtOAc (3×). The combined organic layers were washed with brine (3×), dried over Na₂SO₄, filtered, and evaporated. Purification by FC (SiO₂; PE/EtOAc) gave the title compound (1.45 g, 85% yield) as a colorless oil. MS (ESI): m/z=258.2 [M-C₄H₈+H]⁺

In analogy to Example D.225, the following building blocks were generated using the relevant commercial building blocks and tert-butyl 3-hydroxyazetidine-h-carboxylate (CAS RN: 141699-55-0) in Step a).

| | | | | MS, |
|---|---|---|---|---|
| | | | Building | ESI: |
| Ex. | Structure | Systematic Name | Blocks | m/z |
| D.232 | 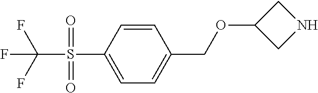 [SMILES: O=S(=O)(c1ccc(COC2CNC2)cc1)C(F)(F)F] | 4- methylbenzenesulfonic acid; 3-[[4- (trifluoromethyl- sulfonyl)phenyl] methoxy]azetidine | 1- (Bromo- methyl)-4- iodobenzene (CAS RN: 16004-15-2) | 295.7 [M − Tos + H]⁺ |
| | 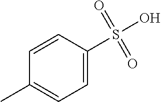 | | | |
| D.237 | 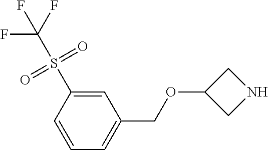 [SMILES: O=S(=O)(c1cccc(COC2CNC2)c1)C(F)(F)F] | 4- methylbenzenesulfonic acid;3-[[3- (trifluoromethyl- sulfonyl)phenyl] methoxy]azetidine | 3- Iodobenzyl bromide (CAS RN: 49617-83-6) | 296.3 [M − Tos + H]⁺ |
| | 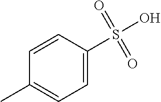 | | | |

### Example D.226

### 2-(azetidin-3-yloxy)-6-cyclopropyl-benzonitrile;4-methylbenzenesulfonic acid

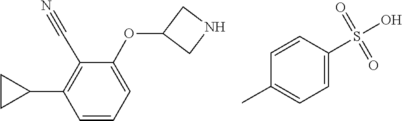 [SMILES: N#Cc1c(OC2CNC2)cccc1C1CC1 ; Cc1ccc(S(=O)(=O)O)cc1]

To a solution of tert-butyl 3-(2-cyano-3-cyclopropylphenoxy)azetidine-1-carboxylate (220 mg, 700 μmol) in EtOAc (1.67 mL) was added 4-methylbenzenesulfonic acid monohydrate (140 mg, 735 μmol). The reaction mixture was refluxed (80° C.) for 16 h, before being cooled down and evaporated, to give the title compound (268 mg, 94% yield) as a white solid. MS (ESI): m/z=215.1 [M-TsOH+H]⁺

### Step a): tert-butyl 3-(3-bromo-2-cyano-phenoxy)azetidine-1-carboxylate

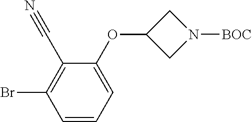 [SMILES: CC(C)(C)OC(=O)N1CC(Oc2cccc(Br)c2C#N)C1]

A solution of 2-bromo-6-hydroxybenzonitrile (CAS RN: 73289-85-7; 450 mg, 2.27 mmol) and tert-butyl 3-hydroxyazetidine-1-carboxylate (CAS RN: 141699-55-0; 394 mg, 2.27 mmol) in dry toluene (7.1 mL) was sparged with Ar, before being treated with (tributylphosphoranylidene)acetonitrile (918 μL, 3.41 mmol). The mixture was stirred at for 2 h at 100° C., before being diluted with EtOAc and washed with 1 M aqueous NaHCO₃ solution. The organic phase was collected and the aqueous phase underwent back-extraction with EtOAc. The combined organic layers were washed with brine, dried over Na₂SO₄, and evaporated. Purification by FC (SiO₂; heptane/EtOAc) gave the title compound (488 mg, 58% yield) as a light brown viscous oil. MS (ESI): m/z=299.0 [M−tBu+H]⁺

### Step b): tert-butyl 3-(2-cyano-3-cyclopropyl-phenoxy)azetidine-1-carboxylate

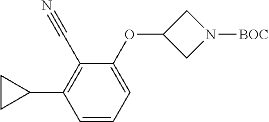 [SMILES: CC(C)(C)OC(=O)N1CC(Oc2cccc(C3CC3)c2C#N)C1]

A solution of tert-butyl 3-(3-bromo-2-cyanophenoxy)azetidine-1-carboxylate (488 mg, 1.38 mmol) in 10:1 1,4-dioxane/water (10 mL) was treated with cyclopropylboronic acid (178 mg, 2.07 mmol) and K₂CO₃ (382 mg, 2.76 mmol), at 23° C. under Ar. The mixture was sparged with Ar, before being treated with bis(triphenylphosphine)palladium (II) chloride (97 mg, 138 μmol). The mixture was heated to 90° C. and stirred for 18 h at this temperature, before being cooled down and diluted with EtOAc. The mixture was washed with water, and the organic layer was washed with brine, dried over Na₂SO₄, filtered, and evaporated. Purification by FC (SiO₂; heptane/EtOAc) gave the title compound (220 mg, 48% yield) as a colorless viscous oil. MS (ESI): m/z=259.2 [M−tBu+H]⁺

In analogy to Example D.226, the following building blocks were generated using the relevant commercial building blocks and tert-butyl 3-hydroxyazetidine-1-carboxylate (CAS RN: 141699-55-0) in Step a).

| | | | Building | MS, |
|---|---|---|---|---|
| Ex. | Structure | Systematic Name | Blocks | ESI: m/z |
| D.229 | 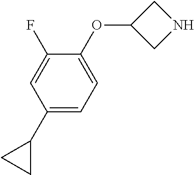 [SMILES: Fc1cc(C2CC2)ccc1OC1CNC1] | 3-(4-cyclopropyl-2- fluoro- phenoxy)azetidine;4- methylbenzenesulfonic acid | 4-bromo-2- fluorophenol (CAS RN: 2105-94-4) | 208.1 [M − Tos + H]⁺ |
| | 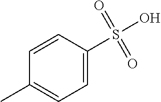 | | | |
| D.233 | 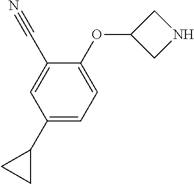 [SMILES: N#Cc1cc(C2CC2)ccc1OC1CNC1] | 2-(azetidin-3-yloxy)-5- cyclopropyl- benzonitrile;4- methylbenzenesulfonic acid | 5-Bromo-2- hydroxybenzo- nitrile (CAS RN: 40530-18- 5) | 215.2 [M − Tos + H]⁺ |
| | 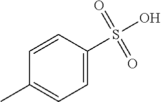 | | | |
| D.235 | 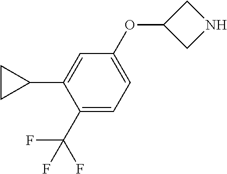 [SMILES: FC(F)(F)c1ccc(OC2CNC2)cc1C1CC1] | 3-[3-cyclopropyl-4- (trifluoromethyl) phenoxy] azetidine;4- methylbenzenesulfonic acid | 3-Bromo-4- (trifluoromethyl) phenol (CAS RN: 1214385- 56-4) | 258.2 [M − Tos + H]⁺ |
| | 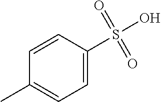 | | | |
| D.238 | 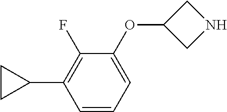 [SMILES: Fc1c(OC2CNC2)cccc1C1CC1] | 3-(3-cyclopropyl-2- fluoro- phenoxy)azetidine;4- methylbenzenesulfonic acid | 3-Bromo-2- fluorophenol (CAS RN: 156682-53-0) | 208.1 [M − Tos + H]⁺ |
| | 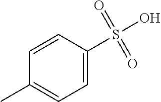 | | | |
| D.243 | 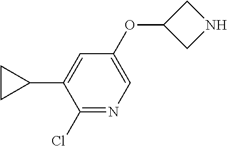 [SMILES: Clc1ncc(OC2CNC2)cc1C1CC1] | 5-(azetidin-3-yloxy)-2- chloro-3-cyclopropyl- pyridine;4- methylbenzenesulfonic acid | 5-bromo-6- chloropyridin- 3-ol (CAS RN: 130115-85-4) | 225.1 [M − Tos + H]⁺ |
| | 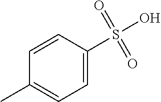 | | | |

### Example D.228

### 3-(4-cyclopropylphenoxy)azetidine;4-methylbenzenesulfonic acid

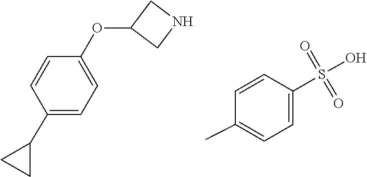

To a solution of tert-butyl 3-(4-cyclopropylphenoxy)azetidine-1-carboxylate (179 mg, 619 μmol) in EtOAc (1.47 mL) was added 4-methylbenzenesulfonic acid monohydrate (124 mg, 650 μmol), at 23° C. The reaction mixture was refluxed for 16 h at 80° C., before being cooled down and evaporated, to give the title compound (220 mg, 94% yield) as a white solid. MS (ESI): m/z=190.1 [M-TsOH+H]⁺

### Step a): tert-butyl 3-(4-cyclopropylphenoxy)azetidine-1-carboxylate

A solution of 4-cyclopropylphenol (CAS RN: 10292-61-2; 150 mg, 1.12 mmol) and tert-butyl 3-hydroxyazetidine-1-carboxylate (CAS RN: 141699-55-0; 194 mg, 1.12 mmol) in dry Toluene (3.49 mL) was sparged with Ar, at 23° C. (Tributylphosphoranylidene)acetonitrile (452 μL, 1.68 mmol) was added, and the mixture was stirred for 2 h at 100° C., before being cooled down, diluted with EtOAc, and washed with 1 M aqueous NaHCO₃. The organic phase was collected and the aqueous phase underwent back-extraction with EtOAc. The combined organic layers were washed with brine, dried over Na₂SO₄, filtered, and evaporated.

Purification by FC (SiO₂; heptane/EtOAc) gave the title compound (179 mg, 53% yield) as a colorless viscous oil. MS (ESI): m/z=234.2 [M−tBu+H]⁺

In analogy to Example D.228, the following building blocks were generated using the relevant commercial building blocks and tert-butyl 3-hydroxyazetidine-1-carboxylate (CAS RN: 141699-55-0) in Step a).

| | | | Building | MS, ESI: |
|---|---|---|---|---|
| Ex. | Structure | Systematic Name | Blocks | m/z |
| D.239 | 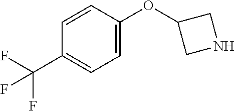 [SMILES: FC(F)(F)c1ccc(OC2CNC2)cc1] | 4-methyl- benzenesulfonic acid; 3-[4- (trifluoromethyl) phenoxy]azetidine | 4- (Trifluoro- methyl)phenol (CAS RN: 402-45-9) | 218.1 [M − Tos + H]⁺ |
| | 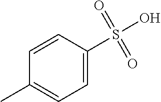 | | | |
| D.297 | 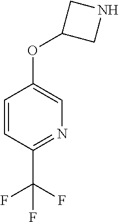 [SMILES: FC(F)(F)c1ccc(OC2CNC2)cn1] | 5-(azetidin-3- yloxy)-2- (trifluoromethyl) pyridine 4- methylbenzene- sulfonate | 6- (trifluoromethyl) pyridin-3-o (CAS: 216766- 12-0) | 219.1 [M − C₇H₈O₃S + H]⁺ |
| | 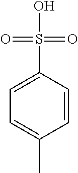 | | | |
| D.298 | 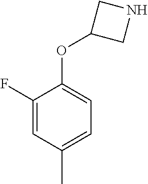 | 3-(2-fluoro-4- methylphenoxy) azetidine 4- methylbenzene- sulfonate | 2-fluoro-4- methylphenol (CAS: 452-85- 7) | 182.1 [M − C₇H₈O₃S + H]⁺ |
| | 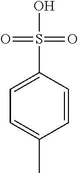 | | | |
| D.299 | 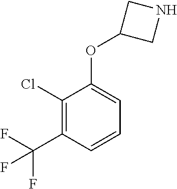 [SMILES: FC(F)(F)c1cccc(OC2CNC2)c1Cl] | 3-(2-chloro-3- (trifluoromethyl) phenoxy)azetidine 4- methylbenzene- sulfonate | 2-chloro-3- (trifluoromethyl) phenol (CAS: 138377-34-1) | 252.1 [M − C₇H₈O₃S + H]⁺ |
| | 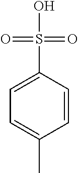 | | | |
| D.300 | 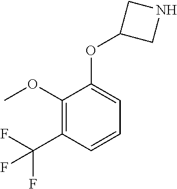 [SMILES: COc1c(OC2CNC2)cccc1C(F)(F)F] | 3-(2-methoxy-3- (trifluoromethyl) phenoxy)azetidine 4- methylbenzene- sulfonate | 2-methoxy-3- (trifluoromethyl) phenol (CAS: 1214334-48-1) | 248.1 [M − C₇H₈O₃S + H]⁺ |
| | 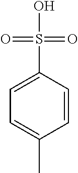 | | | |

### Example D.234

### methyl 2-[3-(azetidin-3-yloxy)phenyl]-2-methyl-propanoate;4-methylbenzenesulfonic acid

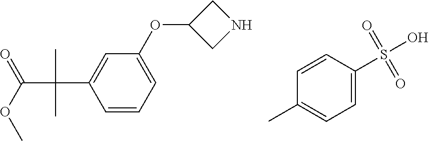 [SMILES: COC(=O)C(C)(C)c1cccc(OC2CNC2)c1 ; Cc1ccc(S(=O)(=O)O)cc1]

A solution of tert-butyl 3-[3-(2-methoxy-1,1-dimethyl-2-oxo-ethyl)phenoxy]azetidine-1-carboxylate (2.0 g, 5.72 mmol) and p-toluenesulfonic acid (1182.76 mg, 6.87 mmol) in EtOAc (25 mL) was stirred at 80° C. for 12 h. The mixture was filtered, and the crystalline solid was washed with EtOAc (10 mL) and dried, to give the title compound (2327 mg, 95.6% yield) as a grey solid. MS (ESI): m/z=250.4 [M-TsOH+H]⁺

### Step a): tert-butyl 3-[3-(2-methoxy-1,1-dimethyl-2-oxo-ethyl)phenoxy]azetidine-]-carboxylate

To a mixture of methyl 2-(3-bromophenyl)-2-methyl-propanoate (CAS RN: 251458-15-8; 5.0 g, 19.45 mmol), tert-butyl 3-hydroxyazetidine-1-carboxylate (CAS RN: 141699-55-0; 3.4 g, 19.45 mmol) and K₃PO₄ (8.25 g, 38.89 mmol) in toluene (100 mL) was added tBuXphos-G3-Pd (772.37 mg, 0.970 mmol), at 23° C. under N₂. The mixture was stirred at 110° C. for 12 h, before being cooled down, filtered, and evaporated. Purification by FC (SiO₂; PE/EtOAc) gave the title compound (4.10 g, 60.34% yield) as a light yellow oil. MS (ESI): m/z=250.5 [M-Boc+H]⁺

### Example D.245

### 3-[(2-fluoro-4-methylsulfonyl-phenyl)methoxy]azetidine;4-methylbenzenesulfonic acid

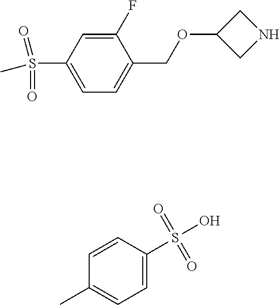 [SMILES: CS(=O)(=O)c1ccc(COC2CNC2)c(F)c1 ; Cc1ccc(S(=O)(=O)O)cc1]

PTSA (1.08 g, 5.66 mmol) was dissolved in isopropyl acetate (10 mL) and heated up to 80° C. A solution of 3-(2-fluoro-4-mesyl-benzyl)oxyazetidine-1-carboxylic acid tert-butyl ester (1.85 g, 5.15 mmol) in isopropyl acetate (10 mL) was added to the reaction mixture at 80° C. The mixture was stirred for 1.5 h at this temperature, before being cooled down to 0° C. and filtered over sintered glass, washed (2× isopropyl acetate) and dried, to give the title compound (1.76 g, 71.32%) as a white solid. MS (ESI): m/z=260.2 [M+H]⁺

### Step a): 1-(chloromethyl)-2-fluoro-4-methylsulfonyl-benzene

A solution of SOCl₂ (1.64 g, 1.01 mL, 13.81 mmol) and tetrabutylammonium chloride (72.13 mg, 0.260 mmol) in DCM (10 mL) was heated to 45° C. and stirred for 5 min at this temperature, before being treated dropwise with (2-fluoro-4-mesyl-phenyl)methanol (CAS RN: 1461702-87-3; 1.06 g, 5.19 mmol). The mixture was stirred for another 2.5 h at 45° C., before being cooled to 0° C. and quenched with 10 mL of water. The resulting solution was stirred for 10 min at 23° C., and poured into a saturated aqueous solution of NaHCO₃ (strong gas evolution). The mixture was poured into DCM, and the organic layer was washed with water and brine, dried over Na₂SO₄, filtered, and evaporated, to give the title compound (1.14 g, 88.78%) as a crude yellow solid, which was directly used in the next step.

### Step b): tert-butyl 3-[(2-fluoro-4-methylsulfonyl-phenyl)methoxy]azetidine-1-carboxylate

To a solution of 3-hydroxyazetidine-1-carboxylic acid tert-butyl ester (886.82 mg, 5.12 mmol) and tetrabutylammonium chloride (71.15 mg, 0.256 mmol) in THF (5 mL) was added.sodium hydroxide (2.19 g, 1.67 mL, 15.36 mmol), at 23° C. The mixture was heated up to 60° C. and a solution of 1-(chloromethyl)-2-fluoro-4-mesyl-benzene (1.14 g, 5.12 mmol) in THF (5 mL) was added. The mixture was stirred for 1.5 h at 60° C., before being cooled down and treated with citric acid 20% (pH 8). The mixture was poured into EtOAc and washed with water and brine. The organic layer was dried over Na₂SO₄, filtered and evaporated, to give the title compound (1.85 g, 90.48%) as a yellow viscous oil. MS (ESI): m/z=304.2 [M+H]⁺

### Example D.251

### 3-[4-(4-chloro-2-methylsulfonyl-phenyl)phenyl]azetidine;4-methylbenzenesulfonic acid

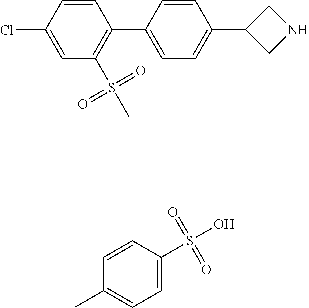 [SMILES: CS(=O)(=O)c1cc(Cl)ccc1-c1ccc(C2CNC2)cc1 ; Cc1ccc(S(=O)(=O)O)cc1]

A solution of tert-butyl 3-[4-(4-chloro-2-methylsulfonyl-phenyl)phenyl]azetidine-1-carboxylate (100.0 g, 237 mmol) and PTSA (44.89 g, 260.7 mmol) in EtOAc (1.7 L) was stirred at 80° C. for 12 h, before being filtered. The cake was washed with EtOAc (1 L) and dried under vacuum, to give the title compound (54 g, 70.8% yield) as a white solid. MS (ESI): m/z=322.1 [M-TsOH+H]⁺

### Step a): tert-butyl 3-(4-bromophenyl)azetidine-1-carboxylate

To a mixture of 4-bromophenylboronic acid (CAS RN: 5467-74-3; 200.04 g, 996.08 mmol) and tert-butyl 3-iodoazetidine-1-carboxylate (CAS RN: 254454-54-1; 141.0 g, 498.04 mmol) in 2-propanol (500 mL) was added rac-(1R,2R)-2-aminocyclohexan-1-ol (3.44 g, 29.88 mmol), and nickel(II) iodide (9.34 g, 29.88 mmol). A mixture of sodium bis(trimethylsilyl)amide in THF (1 L, 1000 mmol) was added slowly to the reaction mixture, under N₂ and keeping the temperature below 30° C. After the resulting mixture was stirred at 25° C. for 30 min, the mixture was heated to 80° C. and stirred for 12 h. The reaction mixture was poured onto H₂O (3 L) and EtOAc(3 L), and the layers were separated. The aqueous layer was extracted twice with EtOAc (2×2 L). The combined organic layers were evaporated, and purified by FC (SiO₂; PE/EtOAc), to give the title compound (140 g, 90.04% yield) as an off-white oil. MS (ESI): m/z=256.1 [M−tBu+H]⁺

### Step b): tert-butyl 3-[4-(4-chloro-2-fluoro-phenyl)phenyl]azetidine-1-carboxylate

To a solution of tert-butyl 3-(4-bromophenyl)azetidine-1-carboxylate (80.0 g, 256.25 mmol), 4-chloro-2-fluorophenylboronic acid (89.36 g, 512.49 mmol) and Na₂CO₃ (54.32 g, 512.49 mmol) in 1,4-Dioxane (1.6 L mL) and water (160 mL) was added Pd(PPh₃)₂Cl₂ (9.37 g, 12.81 mmol). The mixture was then stirred for 12 h at 100° C. under N₂, before being filtered. The filtrate was evaporated, and the residue was treated with water (2 L), extracted with DCM (3×2 L). The combined organic layers were washed with brine, dried over Na₂SO₄, filtered, and evaporated. Trituration with PE (300 mL) at 25° C. gave the title compound (50 g, 53.93% yield). MS (ESI): m/z=306.1 [M−tBu+H]⁺

### Step c): tert-butyl 3-[4-(4-chloro-2-methylsulfanyl-phenyl)phenyl]azetidine-1-carboxylate

To a solution of tert-butyl 3-[4-(4-chloro-2-fluoro-phenyl)phenyl]azetidine-1-carboxylate (150.0 g, 414.55 mmol) in DMSO (1.5 L) was added NaSMe (63.14 g, 900.82 mmol), at 0° C. The mixture was stirred for 12 h at 25° C., before being poured into water (5 L). The aqueous layer was extracted with EtOAc 83×3 L), and poured into saturated aqueous NaClO₄. The combined organic layers were washed with brine, dried over Na₂SO₄, filtered, and evaporated, to give the title compound (130 g, 80.42% yield) as a yellow oil. MS (ESI): m/z=334.1 [M-tBu+H]⁺

### Step d): tert-butyl 3-[4-(4-chloro-2-methylsulfonyl-phenyl)phenyl]azetidine-1-carboxylate

To a solution of tert-butyl 3-[4-(4-chloro-2-methylsulfanyl-phenyl)phenyl]azetidine-1-carboxylate (130.0 g, 333.38 mmol) in DCM (1.3 L) was slowly added 3-chloroperoxybenzoic acid (172.6 g, 1000 mmol), at 25° C. The mixture was stirred for 4 h at this temperature, before being washed with saturated aqueous Na₂SO₃ (3×2 L). The aqueous layer was extracted with EtOAc 3×3 L). The combined organic layers were dried over Na₂SO₄, filtered, and evaporated. Purification by FC (SiO₂; PE/EtOAc) gave the title compound (100 g, 71.1% yield) as a yellow oil. MS (ESI): m/z=365.8 [M−tBu+H]⁺

### Example D.254

### 2-[4-(azetidin-3-yl)phenyl]benzamide;4-methylbenzenesulfonic acid

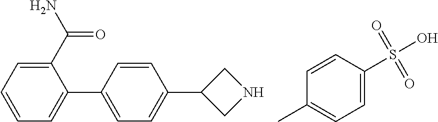

A solution of tert-butyl 3-[4-(2-carbamoylphenyl)phenyl]azetidine-1-carboxylate (350.0 mg, 0.990 mmol) and p-toluenesulfonic acid monohydrate (377.82 mg, 1.99 mmol) in EtOAc (30 mL) was stirred at 80° C. for 3 h, before being evaporated. Purification by RP-HPLC gave the title compound (166.7 mg, 38.75% yield) as a yellow viscous oil. MS (ESI): m/z=253.2 [M+H]⁺

### Step a): tert-butyl 3-(4-bromophenyl)azetidine-1-carboxylate

To a mixture of 4-bromophenylboronic acid (CAS RN: 5467-74-3; 200.04 g, 996.08 mmol) and tert-butyl 3-iodoazetidine-1-carboxylate (CAS RN: 254454-54-1; 141.0 g, 498.04 mmol) in 2-propanol (500 mL) was added rac-(1R,2R)-2-aminocyclohexan-1-ol (3.44 g, 29.88 mmol), and nickel(II) iodide (9.34 g, 29.88 mmol). A mixture of sodium bis(trimethylsilyl)amide in THF (1 L, 1000 mmol) was added slowly to the reaction mixture, under N₂ and keeping the temperature below 30° C. After the resulting mixture was stirred at 25° C. for 30 min, the mixture was heated to 80° C. and stirred for 12 h. The reaction mixture was poured onto H₂O (3 L) and EtOAc(3 L), and the layers were separated. The aqueous layer was extracted twice with EtOAc (2×2 L). The combined organic layers were evaporated, and purified by FC (SiO₂; PE/EtOAc), to give the title compound (140 g, 90.04% yield) as an off-white oil. MS (ESI): m/z=256.1 [M−tBu+H]⁺

### Step b): tert-butyl 3-[4-(2-methoxycarbonylphenyl)phenyl]azetidine-]-carboxylate

A solution of tert-butyl 3-(4-bromophenyl)azetidine-1-carboxylate (2.1 g, 6.73 mmol), 2-methoxycarbonylphenylboronic acid (1.69 g, 9.42 mmol) and cesium carbonate (3.29 g, 10.09 mmol) in 1,4-Dioxane (55 mL) and Water (3 mL) was sparged with Argon for 5 min, before being treated with 1,1′-bis(diphenylphosphino)ferrocene-palladium(II)dichloride dichloromethane complex (439.1 mg, 0.540 mmol). The mixture was stirred for 18 h at 95° C., before being cooled down, filtered over silica, and washed with 1,4-dioxane. The filtrate was evaporated, and purification by FC (SiO₂; PE/EtOAc) gave the title compound (1.1 g, 42.28% yield) as a light yellow oil. MS (ESI): m/z=312.1 [M−tBu+H]⁺

### Step c): 2-[4-(]-tert-butoxycarbonylazetidin-3-yl)phenyl]benzoic acid

To a solution of tert-butyl 3-[4-(2-methoxycarbonylphenyl)phenyl]azetidine-1-carboxylate (600.0 mg, 1.63 mmol) in 4:4:1 THF/MeOH/water (22.5 mL) was added lithium hydroxide monohydrate (342.59 mg, 8.16 mmol). The mixture was stirred for 18 h at 25° C., before being evaporated and acidified to pH 4 with a saturated solution of citric acid. The resulting suspension was stirred for 30 min and filtered. The cake was washed with water and dried, to give the title compound (350 mg, 57.62% yield) as a white solid. MS (ESI): m/z=352.2 [M−H]⁻

### Step d): tert-butyl 3-[4-(2-carbamoylphenyl)phenyl]azetidine-]-carboxylate

A solution of 2-[4-(1-tert-butoxycarbonylazetidin-3-yl)phenyl]benzoic acid (400.0 mg, 1.13 mmol) in THF (10 mL) was treated with CDI (162.15 mg, 1.47 mmol), at 23° C. The mixture was stirred for 3 h at this temperature, before being treated with ammonium hydroxide (3 mL, 29% aqueous solution). The mixture was stirred for 18 h at this temperature, before being evaporated. The residue was partitioned between EtOAc (50 mL) and water (20 mL). The organic layer was dried over Na₂SO₄, filtered, and evaporated, to give the title compound (350 mg, 83.36% yield) as a white waxy solid. MS (ESI): m/z=350.8 [M−H]⁻

### Example D.255

### N-[[3-(azetidin-3-yl)-1-bicyclo[1.1.1]pentanyl]methyl]-1-(trifluoromethyl)cyclopropanamine;4-methylbenzenesulfonic acid

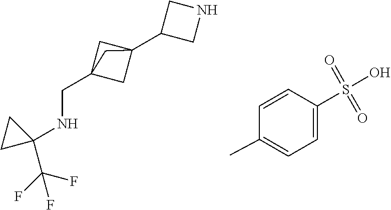 [SMILES: FC(F)(F)C1(NCC23CC(C4CNC4)(C2)C3)CC1 ; Cc1ccc(S(=O)(=O)O)cc1]

A solution of tert-butyl 3-[3-[[[1-(trifluoromethyl)cyclopropyl]amino]methyl]-1-bicyclo[1.1.1]pentanyl]azetidine-1-carboxylate (380.0 mg, 1.05 mmol) in EtOAc (25 mL) was treated with p-toluenesulfonic acid monohydrate (441.23 mg, 2.32 mmol), at 23° C. The mixture was stirred for 16 h at 50° C., before being cooled down to 0° C. for 1 h. The resulting precipitate was collected by filtration and washed twice with MTBE (2*100 mL), to give the title compound (607.1 mg, 90.46% yield) as a light yellow solid. MS (ESI): m/z=261.2 [M+H]⁺

### Step a): tert-butyl 3-[3-(hydroxymethyl)-1-bicyclo[1.1.1]pentanyl]azetidine-1-carboxylate

A solution of 3-(1-tert-butoxycarbonylazetidin-3-yl)bicyclo[1.1.1]pentane-1-carboxylic acid (CAS RN: 2227205-20-9; 6.0 g, 22.45 mmol) in THF (70 mL) was treated with borane-methyl sulfide complex (4262.87 mg, 56.11 mmol), at 0° C. The mixture was stirred at reflux for 6 h, before being cooled down to 0° C., treated dropwise with MeOH (5 mL), and evaporated. The residue was diluted with brine, and extracted with EtOAc (3×). The organic layers were combined, dried over Na₂SO₄, filtered and evaporated, to give the title compound (5.4 g, 90.22% yield) as a white solid. MS (ESI): m/z=198.0 [M-Boc+H]⁺

### Step b): tert-butyl 3-(3-formyl-1-bicyclo[1.1.]pentanyl)azetidine-1-carboxylate

To a solution of tert-butyl 3-[3-(hydroxymethyl)-1-bicyclo[1.1.1]pentanyl]azetidine-1-carboxylate (1.2 g, 4.74 mmol) in DCM (50 mL) was added DMP (2.4 g, 5.68 mmol), at 0° C. The mixture was allowed to warm up to 23° C. and was stirred for 3 h at this temperature. The mixture was treated with saturated aqueous sodium sulfite (40 mL) and extracted with DCM (2×20 mL), washed with saturated aqueous NaHCO₃ and brine (2×20 mL), dried over Na₂SO₄, filtered and evaporated, to give the title compound (1.1 g, 64.68% yield) as a colorless viscous oil, which was directly used in the next step.

### Step c): tert-butyl 3-[3-[[[ ]-(trifluoromethyl)cyclopropyl]amino]methyl]-1-bicyclo[1.1.1]pentanyl]azetidine-1-carboxylate

A solution of tert-butyl 3-(3-formyl-1-bicyclo[1.1.1]pentanyl)azetidine-1-carboxylate (1.1 g, 3.06 mmol) and 1-(trifluoromethyl)cyclopropanamine hydrochloride (544.46 mg, 3.37 mmol) in 1,2-dichloroethane (50 mL) was stirred for 2 h at 23° C., before being treated with sodium triacetoxyborohydride (1.9 g, 9.19 mmol). The mixture was stirred at this temperature for another 18 h, before being treated with saturated aqueous NaHCO₃. The mixture was extracted with DCM. The combined organic layers were dried over Na₂SO₄, filtered, and evaporated, to give the title compound (380 mg, 34.41% yield) as a clear oil. MS (ESI): m/z=361.2 [M+H]⁺

### Example D.256

### 3-(azetidin-3-yl)-N-[[1-(trifluoromethyl)cyclopropyl]methyl]bicyclo[1.1.1]pentan-1-amine;4-methylbenzenesulfonic acid

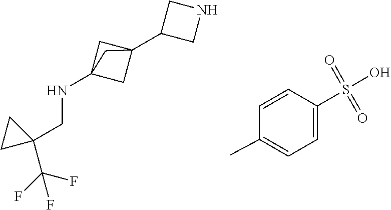 [SMILES: FC(F)(F)C1(CNC23CC(C4CNC4)(C2)C3)CC1 ; Cc1ccc(S(=O)(=O)O)cc1]

A solution of tert-butyl 3-[3-[[1-(trifluoromethyl)cyclopropyl]methylamino]-1-bicyclo[1.1.1]pentanyl]azetidine-1-carboxylate (800.0 mg, 2.22 mmol) in EtOAc (50 mL) was treated with p-toluenesulfonic acid monohydrate (928.91 mg, 4.88 mmol), at 23° C. The mixture was stirred for 16 h at this temperature, before being cooled down to 0° C. for 1 h. The precipitate was filtered, washed with MTBE, and dried, to give the title compound (1191 mg, 84.26% yield) as a white solid. MS (ESI): m/z=261.2 [M+H]⁺

### Step a): tert-butyl 3-[3-(benzyloxycarbonylamino)-1-bicyclo[1.1.1]pentanyl]azetidine-]-carboxylate

A solution of 3-(1-tert-butoxycarbonylazetidin-3-yl)bicyclo[1.1.1]pentane-1-carboxylic acid (CAS RN: 2227205-20-9; 2.0 g, 7.48 mmol) and benzyl alcohol (1.6 g, 14.96 mmol) in toluene (50 mL) was treated with TEA (3.13 mL, 22.45 mmol), at 23° C. The mixture was stirred for 5 min at this temperature, and diphenylphosphonic azide (1.69 mL, 7.86 mmol) was added. The mixture was stirred for another 15 min at 23° C., and for 16 h at 100° C. The mixture was cooled down, poured into ice-cold water (50 mL), and extracted with MTBE. The organic layer was washed with water and brine, dried over Na₂SO₄, filtered, and evaporated.

Purification by FC (SiO₂; hexane/MTBE) gave the title compound (2.35 g, 80.11% yield) as a colorless viscous oil. MS (ESI): m/z=273.0 [M-Boc+H]⁺

### Step b): tert-butyl 3-(3-amino-]-bicyclo[1.1.1]pentanyl)azetidine-1-carboxylate

To a solution of tert-butyl 3-[3-(benzyloxycarbonylamino)-1-bicyclo[1.1.1]pentanyl]azetidine-1-carboxylate (2200.0 mg, 5.91 mmol) in MeOH (50 mL) was added palladium (10% on C) (0.21 mL, 0.210 mmol). The reaction mixture was stirred for 24 h at 23° C. under hydrogen atmosphere, before being put back under Ar and filtered. The filtrate was evaporated, and the residue was triturated with MTBE (100 mL). The precipitate was collected by filtration, to give the title compound (600 mg, 40.49% yield) as a white solid. MS (ESI): m/z=239.2 [M+H]⁺

### Step c): tert-butyl 3-[3-[[ ]-(trifluoromethyl)cyclopropyl]methylamino]-1-bicyclo[1.1.1]pentanyl]azetidine-]-carboxylate

A solution of tert-butyl 3-(3-amino-1-bicyclo[1.1.1]pentanyl)azetidine-1-carboxylate (1.0 g, 4.2 mmol) and 1-(trifluoromethyl)cyclopropanecarbaldehyde (521.47 mg, 3.78 mmol) in 1,2-dichloroethane (50 mL) was stirred for 2 h at 23° C., before being treated with sodium triacetoxyborohydride (2.6 g, 12.59 mmol). The mixture was stirred for another 18 h at this temperature, before being treated with saturated aqueous NaHCO₃. The mixture was extracted with DCM (2×20 mL), and the combined organic layers were dried over Na₂SO₄, filtered, and evaporated. Purification by FC (SiO₂; hexane/MTBE) gave the title compound (800 mg, 50.26% yield) as a white solid. MS (ESI): m/z=361.2 [M+H]⁺

### Example D.258

### 2-[3-(azetidin-3-yl)-1-bicyclo[1.1.1]pentanyl]-5-(2,2,2-trifluoroethyl)-1,3,4-oxadiazole;2,2,2-trifluoroacetic acid

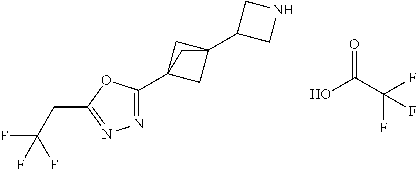 [SMILES: FC(F)(F)Cc1nnc(C23CC(C4CNC4)(C2)C3)o1 ; O=C(O)C(F)(F)F]

To a solution of tert-butyl 3-[3-[5-(2,2,2-trifluoroethyl)-1,3,4-oxadiazol-2-yl]-1-bicyclo[1.1.1]pentanyl]azetidine-1-carboxylate (2.0 g, 5.36 mmol) in DCM (200 mL) was added TFA (2.06 mL, 26.78 mmol), at 25° C. The mixture was stirred for 18 h at this temperature, before being evaporated. The residue was treated with TBME (200 mL), and the resulting precipitate was filtered, washed with TBME (2×50 mL), and dried, to give the title compound (1.8 g, 81.3% yield) as a light yellow solid. MS (ESI): m/z=274.0 [M+H]⁺

### Step a): tert-butyl 3-[3-[(3,3,3-trifluoropropanoylamino)carbamoyl]-1-bicyclo[1.1.1]pentanyl]azetidine-1-carboxylate

To a stirred solution of 3-(1-tert-butoxycarbonylazetidin-3-yl)bicyclo[1.1.1]pentane-1-carboxylic acid (CAS RN: 1211526-53-2; 4.0 g, 14.96 mmol) in DCM (120 mL) was added CDI (2.79 g, 17.21 mmol), at 25° C. The mixture was stirred for 30 min at this temperature, before being treated with 3,3,3-trifluoropropanehydrazide (CAS RN: 934171-99-0; 2.23 g, 15.71 mmol). The mixture was stirred for another 18 h, before being diluted with DCM (150 mL). The organic layer was washed with water, dried over Na₂SO₄, filtered and evaporated, to give the title compound (5.8 g, 97.05% yield) as a white solid. MS (ESI): m/z=390.2 [M−H]⁻

### Step b): tert-butyl 3-[3-[5-(2,2,2-trifluoroethyl)-],3,4-oxadiazol-2-yl]-1-bicyclo[1.1.1]pentanyl]azetidine-1-carboxylate

To a stirred solution of tert-butyl 3-[3-[(3,3,3-trifluoropropanoylamino)carbamoyl]-1-bicyclo[1.1.1]pentanyl]azetidine-1-carboxylate (5.8 g, 14.82 mmol) and DIPEA (7.74 mL, 44.46 mmol) in ACN (200 mL) was added p-toluenesulfonyl chloride (3.67 g, 19.26 mmol). The mixture was stirred for 24 h at 50° C., before being evaporated. Purification by FC (SiO₂; PE/TBME) gave the title compound (4.2 g, 72.11% yield) as a light brown solid. MS (ESI): m/z=318.0 [M−tBu+H]⁺

In analogy to Example D.258, the following building block was generated using the relevant commercial building blocks and 3-(1-tert-butoxycarbonylazetidin-3-yl)bicyclo[1.1.1]pentane-1-carboxylic acid (CAS RN: 1211526-53-2) in Step a).

| | | | Building | MS, ESI: |
|---|---|---|---|---|
| Ex. | Structure | Systematic Name | Blocks | m/z |
| D.259 | 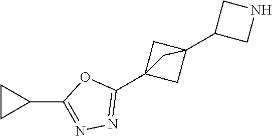 [SMILES: C1CC1c1nnc(C23CC(C4CNC4)(C2)C3)o1] | 2-[3-(azetidin-3-yl)-1- bicyclo[1.1.1] pentanyl-5- cyclopropyl-1,3,4- oxadiazole;2,2,2- trifluoroacetic acid | cyclopropane- carbohydrazide (CAS RN: 6952-93-8) | 232.2 [M + H]⁺ |
| | 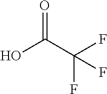 | | | |

### Example D.268

### N-(2-azaspiro[3.3]heptan-6-ylmethyl)-3-(trifluoromethyl)oxetan-3-amine;4-methylbenzenesulfonic acid

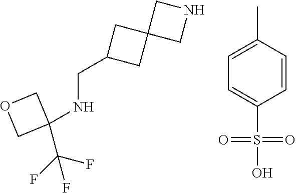 [SMILES: FC(F)(F)C1(NCC2CC3(CNC3)C2)COC1 ; Cc1ccc(S(=O)(=O)O)cc1]

A solution of tert-butyl 6-[[[3-(trifluoromethyl)oxetan-3-yl]amino]methyl]-2-azaspiro[3.3]heptane-2-carboxylate (270.0 mg, 0.770 mmol) in 4:1 MTBE/ACN (25 mL) was treated with PTSA (366.45 mg, 1.93 mmol), at 23° C. The mixture was stirred for 18 h at 40° C., and the resulting precipitate was filtered off, washed with Et₂O and dried, to give the title compound (187.7 mg, 38.9% yield) as a white solid. MS (ESI): m/z=251.0 [M+H]⁺

### Step a): tert-butyl 6-[[[3-(trifluoromethyl)oxetan-3-yl]amino]methyl]-2-azaspiro[3.3]heptane-2-carboxylate

A solution of 3-(trifluoromethyl)oxetan-3-amine;hydrochloride (394.07 mg, 2 mmol) in DCE (15 mL) was treated with tert-butyl 6-formyl-2-azaspiro[3.3]heptane-2-carboxylate (CAS RN: 1440960-67-7; 300.0 mg, 1.3 mmol) and TEA (0.24 mL, 1.73 mmol), at 23° C. The mixture was stirred for 10 min at this temperature, before being treated with acetic acid (159.94 mg, 2.66 mmol). The mixture was stirred for another 60 min, and sodium triacetoxyborohydride (705.59 mg, 3.33 mmol) was added. The mixture was stirred for another 18 h, before being treated with saturated aqueous NaHCO₃. The mixture was extracted with DCM (2×50 mL). The combined organic layers were washed with brine, dried over Na₂SO₄, filtered, and evaporated, to give the title compound (390 mg, 71.05% yield) as a light yellow solid. MS (ESI): m/z=351.2 [M+H]⁺

### Example D.275

### 2-(4-fluorophenyl)-2-(4-piperidyl)acetamide;4-methylbenzenesulfonic acid

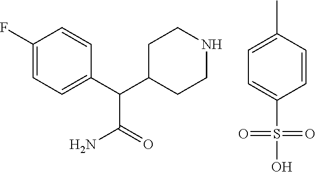

PTSA (267.17 mg, 1.4 mmol) was added to a stirred solution of tert-butyl 4-[2-amino-1-(4-fluorophenyl)-2-oxo-ethyl]piperidine-1-carboxylate (450.0 mg, 1.34 mmol) in EtOAc (40 mL). The mixture was heated for 16 h at 70° C., before being cooled down and evaporated. Trituration with MTBE gave the title compound (340.2 mg, 61.57% yield) as a light brown solid. MS (ESI): m/z=237.2 [M+H]⁺

### Step a): tert-butyl 4-[cyano-(4-fluorophenyl)methylene]piperidine-]-carboxylate

To the solution of 2-(4-fluorophenyl)acetonitrile (CAS RN: 459-22-3; 888 μL, 7.4 mmol) in EtOH (37 mL) was added sodium ethanolate (604 mg, 8.88 mmol), at 23° C. The mixture was stirred for 30 min at this temperature, before being treated dropwise with a solution of tert-butyl 4-oxopiperidine-1-carboxylate (CAS RN: 79099-07-3; 1.47 g, 7.4 mmol) in EtOH (37 mL). The mixture was stirred for 18 h at 23° C., before being poured into a saturated aqueous NH₄Cl solution and EtOAc. The layers were separated, and the aqueous layer was extracted with EtOAc (2×). The combined organic layers were dried over MgSO₄, filtered, and evaporated. Purification by FC (SiO₂; heptane/EtOAc) gave the title compound (2.32 g, 99.1% yield) as a colorless solid. MS (ESI): m/z=261.2 [M-C₄H₈+H]⁺

### Step b): tert-butyl 4-[cyano-(4-fluorophenyl)methyl]piperidine-]-carboxylate

A solution of tert-butyl 4-(cyano(4-fluorophenyl)methylene)piperidine-1-carboxylate (526 mg, 1.66 mmol) in 1:1 MeOH/EtOAc (10 mL) was treated with Pd/C 10% (106.2 mg, 100 μmol), at 23° C. The mixture was stirred for 18 h under hydrogen atmosphere at 1.3 bars, before being filtered. The filtrate was evaporated. Purification by FC (SiO₂; heptane/EtOAc) gave the title compound (0.415 g; 78.4% yield) as a colorless gum. MS (ESI): m/z=263.2 [M+H]⁺

### Step c): 2-(4-fluorophenyl)-2-(4-piperidyl)acetic acid

A solution of tert-butyl 4-(cyano(4-fluorophenyl)methyl)piperidine-1-carboxylate (555 mg, 1.74 mmol) in HBr 48% in water (5.55 mL, 49.1 mmol) was stirred at reflux for 4.5 h, before being evaporated. The residue was suspended in 2-propanol (2 mL), homogenized and filtered.

The filter cake was washed three times with 2-propanol (3×1 mL). The mother liquor was completely evaporated and dried for 2 h at high vacuum in the presence of P₂O₅ to yield the title compound (0.535 g; 96.5% yield) as a light brown solid. MS (ESI): m/z=238.2 [M−HBr+H]⁺

### Step d): 2-(1-tert-butoxycarbonyl-4-piperidyl)-2-(4-fluorophenyl)acetic acid

To a turbid solution of 2-(4-fluorophenyl)-2-(piperidin-4-yl)acetic acid hydrobromide (535 mg, 1.55 mmol) in 1 M NaOH (3.09 mL, 3.09 mmol) was added dropwise a solution of Boc₂O (391 μL, 1.68 mmol) in DME (5 mL). The mixture was stirred for 3 h at 23° C., before being evaporated. The residue was taken up in 1.2 mL citric acid 10% in water (pH approx. 4) and ethyl acetate, and the layers were separated. The aqueous layer was extracted once with ethyl acetate. The combined organic layers were dried over MgSO₄, filtered and evaporated, to give the title compound (0.520 g; 99.6% yield) as a light brown solid. MS (ESI): m/z=336.3 [M−H]⁻

### Step e): tert-butyl 4-[2-amino-]-(4-fluorophenyl)-2-oxo-ethyl]piperidine-]-carboxylate

A solution of 2-(1-tert-butoxycarbonyl-4-piperidyl)-2-(4-fluorophenyl)acetic acid (2.0 g, 5.93 mmol) in THF (20 mL) was treated with CDI (1.44 g, 8.89 mmol), at 23° C. The mixture was stirred for 30 min at that temperature, before being treated dropwise with ammonia (25% in water) (1.01 g, 59.28 mmol). The mixture was stirred for another 16 h, before being diluted with EtOAc (40 mL) and washed with water. The organic layer was dried over Na₂SO₄, filtered, and evaporated, to give the title compound (1.9 g, 90.52% yield) as a white solid. MS (ESI): m/z=237.2 [M-Boc+H]⁺

### Example D.280

### 5-[4-(azetidin-3-yl)phenyl]-1-(2,2-dimethylpropyl)triazole;4-methylbenzenesulfonic acid

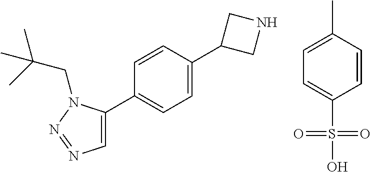 [SMILES: CC(C)(C)Cn1nncc1-c1ccc(C2CNC2)cc1 ; Cc1ccc(S(=O)(=O)O)cc1]

A solution of tert-butyl 3-[4-[3-(2,2-dimethylpropyl)triazol-4-yl]phenyl]azetidine-1-carboxylate (450.0 mg, 1.21 mmol) and p-toluenesulfonic acid monohydrate (346.56 mg, 1.82 mmol) in EtOAc (25 mL) was stirred at 50° C. for 8 h, before being evaporated. The residue was treated with THF (50 mL), and the resulting precipitate was filtered off, washed with THF, and dried, to give the title compound (500.9 mg, 88.5% yield) as a light grey solid. MS (ESI): m/z=271.2 [M+H]⁺

### Step a): 5-(4-bromophenyl)-1-(2,2-dimethylpropyl)triazole

A solution of neopentylamine (CAS RN: 5813-64-9; 5.54 g, 63.4 mmol) in toluene (400 mL) was treated with 4′-bromoacetophenone (CAS RN: 99-90-1; 9.7 g, 48.73 mmol), 1-azido-4-nitro-benzene (CAS RN: 17271-88-4; 8.0 g, 48.73 mmol), 4 A MS, and acetic acid (877.97 mg, 14.62 mmol), at 23° C. The mixture was refluxed for 72 h, before being cooled down, filtered, and evaporated. Purification by FC (SiO₂; CHCl₃/ACN) gave the title compound (6.65 g, 44.06% yield) as a light brown solid. MS (ESI): m/z=294.2/296.2 [M+H]⁺

### Step b): tert-butyl 3-[4-[3-(2,2-dimethylpropyl)triazol-4-yl]phenyl]azetidine-1-carboxylate

To a 40 mL vial equipped with a magnetic stir bar were added tert-butyl 3-bromoazetidine-1-carboxylate (1.0 g, 4.24 mmol), 5-(4-bromophenyl)-1-(2,2-dimethylpropyl)triazole (1.245 g, 4.24 mmol), Ir[dF(CF₃)ppy]₂(dtbbpy)PF₆ (56.97 mg, 0.050 mmol), NiCl₂-glyme (5.58 mg, 0.030 mmol), 4-tert-butyl-2-(4-tert-butyl-2-pyridyl)pyridine (8.18 mg, 0.030 mmol), bis(trimethylsilyl)silyl-trimethyl-silane (1.26 g, 5.08 mmol) and Na₂CO₃ (1.08 g, 10.16 mmol) in DME (40 mL). The vial was sealed and placed under nitrogen. The reaction was stirred and irradiated with a 34 W blue LED lamp (7 cm away) at 25° C. for 14 h, before being filtered and evaporated. Purification by FC (SiO₂; PE/EtOAc) gave the title compound (900 mg, 55.6% yield) as a yellow oil. MS (ESI): m/z=371.4 [M+H]⁺

### Example D.287

### 3-[4-(azetidin-3-yl)phenyl]-5-[1-(trifluoromethyl)cyclopropyl]-4H-1,2,4-triazole;4-methylbenzenesulfonic acid

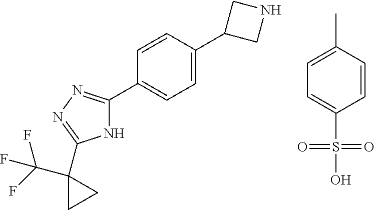 [SMILES: FC(F)(F)C1(c2nnc(-c3ccc(C4CNC4)cc3)[nH]2)CC1 ; Cc1ccc(S(=O)(=O)O)cc1]

To a solution of tert-butyl 3-[4-[5-[1-(trifluoromethyl)cyclopropyl]-4H-1,2,4-triazol-3-yl]phenyl]azetidine-1-carboxylate (700.0 mg, 1.71 mmol) in EtOAc (43.75 mL) was added p-toluenesulfonic acid monohydrate (717.25 mg, 3.77 mmol), at 23° C. The mixture was heated to 47° C. and stirred for 18 h at this temperature, before being evaporated. Trituration with Et₂O gave the title compound (604 mg, 53.99% yield) as a white solid. MS (ESI): m/z=309.0 [M+H]⁺

### Step a): tert-butyl 3-[4-[5-[ ]-(trifluoromethyl)cyclopropyl]-4H-1,2,4-triazol-3-yl]phenyl]azetidine-1-carboxylate

A solution of 1-(trifluoromethyl)cyclopropanecarbohydrazide (CAS RN: 1016557.86-0; 4.07 g, 24.19 mmol), tert-butyl 3-(4-cyanophenyl)azetidine-1-carboxylate (CAS RN: 206446-41-5; 1.25 g, 4.84 mmol) and potassium carbonate (6.69 g, 48.39 mmol) in 1-butanol (112.5 mL) was stirred for 90 h at 150° C., before being cooled down, filtered, and evaporated. Trituration with hexane gave the title compound (700 mg, 34.710% yield) as a white solid. MS (ESI): m/z=409.0 [M+H]⁺

### Example D.289

### 5-[1-[4-(azetidin-3-yl)phenyl]cyclopropyl]-1H-tetrazole;4-methylbenzenesulfonic acid

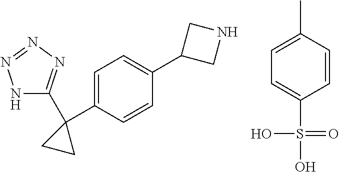

To the solution of tert-butyl 3-[4-[1-(2H-tetrazol-5-yl)cyclopropyl]phenyl]azetidine-1-carboxylate (1.9 g, 4.45 mmol) (80% purity) in EtOAc (30 mL) was added p-toluenesulfonic acid monohydrate (1.0 g, 5.34 mmol). The mixture was stirred for 24 h at 50° C., before being evaporated. Purification by RP-HPLC gave the title compound (323 mg, 15.97% yield) as a light yellow viscous oil. MS (ESI): m/z=242.1 [M+H]⁺

### Step a): tert-butyl 3-[4-(1-cyanocyclopropyl)phenyl]azetidine-1-carboxylate

A vial was charged with tert-butyl 3-bromoazetidine-1-carboxylate (CAS RN: 1064194-10-0; 2.76 g, 11.71 mmol), 1-(4-bromophenyl)cyclopropanecarbonitrile (CAS RN: 124276-67-1; 2.0 g, 9.01 mmol), Ir[dF(CF₃)ppy]₂(dtbbpy)PF₆ (100.95 mg, 0.090 mmol), NiCl₁₂dtbbpy (17.92 mg, 0.050 mmol), Na₂CO₃ (1909.04 mg, 18.01 mmol), bis(trimethylsilyl)silyl-trimethyl-silane (2.24 g, 9.01 mmol) and DCE (40 mL). The vial was sealed and placed under nitrogen. The reaction was stirred and irradiated with a 34 W blue LED lamp (7 cm away) equipped with cooling fan to keep the reaction temperature at 25° C. for 20 h. The mixture was filtered off and evaporated. Purification by RP-HPLC gave the title compound (1.60 g, 59.54% yield) as a yellow oil. MS (ESI): m/z=243.4 [M-C₄H₈+H]⁺

### Step b): tert-butyl 3-[4-[ ]-(2H-tetrazol-5-yl)cyclopropyl]phenyl]azetidine-1-carboxylate

Caution, for this reaction, use a protective shield and carry out all operations in the ventilation hood. To a stirred suspension of tert-butyl 3-[4-(1-cyanocyclopropyl)phenyl]azetidine-1-carboxylate (1.70 g, 5.7 mmol) and dibutyloxostannane (425.5 mg, 1.71 mmol) in dry 1,4-dioxane (30 mL) was added azidotrimethylsilane (3.02 mL, 22.79 mmol), at 23° C. The mixture was heated to 110° C., and stirred for 18 h at this temperature. The mixture was cooled down and evaporated, to give the title compound (1.9 g, 84.98% yield) as a dark brown oil. MS (ESI): m/z=340.2[M−H]⁻

### Example D.291

### 5-(azetidin-3-yl)-N-(4-isopropylphenyl)-N-methyl-pyridin-2-amine;4-methylbenzenesulfonic acid

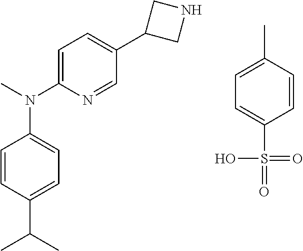 [SMILES: CC(C)c1ccc(N(C)c2ccc(C3CNC3)cn2)cc1 ; Cc1ccc(S(=O)(=O)O)cc1]

A solution of 3-[6-(4-isopropyl-N-methyl-anilino)-3-pyridyl]azetidine-1-carboxylic acid tert-butyl ester (334 mg, 0.875 mmol) and p-toluenesulfonic acid monohydrate (499.59 mg, 2.63 mmol) in EtOAc (3 mL) was heated at reflux for 1 h, before being cooled down to 23° C. and stirred for 18 h at this temperature. The resulting precipitate was filtered off and washed with EtOAc. Purification by FC (Si—NH₂; ACN/MeOH) gave the title compound (0.211 g, 85.6%) as a yellow oil. MS (ESI): m/z=282.3 [M+H]⁺.

### Step a): 5-bromo-N-(4-isopropylphenyl)-N-methyl-pyridin-2-amine

A solution of (5-bromo-2-pyridyl)-p-cumenyl-amine (CAS RN: 107962-10-7; 400 mg, 1.37 mmol) in THF (3.41 mL) was treated with NaH (55% in mineral oil) (71.93 mg, 1.65 mmol), at 0° C. The mixture was stirred for 45 min at this temperature, before being treated with iodomethane (120.25 μL, 1.92 mmol). The mixture was stirred for 46 h at 23° C., before being poured onto water and EtOAc, and the layers were separated. The aqueous layer was extracted with EtOAc. The combined organic layers were dried over MgSO₄, filtered, treated with silica gel, and evaporated. Purification by FC (SiO₂; heptane/EtOAc) gave the title compound (0.363 g; 86.5%) as a colorless oil. MS (ESI): m/z=305.1 [M+H]⁺

### Step b): tert-butyl 3-[6-(4-isopropyl-N-methyl-anilino)-3-pyridyl]azetidine-1-carboxylate

To a sealed vial equipped with a stir bar were added bis[3,5-difluoro-2-[5-(trifluoromethyl)-2-pyridyl]phenyl]iridium(1+);4-tert-butyl-2-(4-tert-butyl-2-pyridyl)pyridine;hexafluorophosphate (13.23 mg, 0.012 mmol), 3-bromoazetidine-1-carboxylic acid tert-butyl ester (417.74 mg, 1.77 mmol), (5-bromo-2-pyridyl)-methyl-p-cumenyl-amine (360 mg, 1.18 mmol), bis(trimethylsilyl)silyl-trimethyl-silane (363.89 μL, 1.18 mmol) and Na₂CO₃ (250.03 mg, 2.36 mmol). The vial was sealed and placed under Ar before ethylene glycol dimethyl ether (3.4 mL) was added. To a separate vial were added dichloronickel;1,2-dimethoxyethane (2.59 mg, 0.012 mmol) and 4-tert-butyl-2-(4-tert-butyl-2-pyridyl)pyridine (3.17 mg, 0.012 mmol). This vial was sealed, purged with Ar, and ethylene glycol dimethyl ether (1 mL) was added. The mixture was sonicated for 5 min, after which 0.5 mL of it was added to the main reaction mixture. The mixture was stirred and irradiated with a 420 nm lamp (75% intensity) for 6 h, before being filtered off. Purification by FC (SiO₂; heptane/EtOAc) gave the title compound (0.334 g; 74.2%) as a light yellow oil. MS (ESI): m/z=382.3 [M+H]⁺

### Example D.294

### 1-[4-(azetidin-3-yl)phenyl]-4,4-difluoro-piperidine-2-carboxamide;4-methylbenzenesulfonic acid

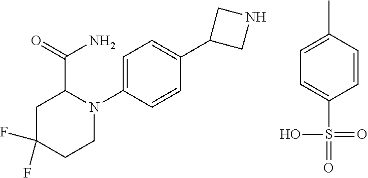 [SMILES: NC(=O)C1CC(F)(F)CCN1c1ccc(C2CNC2)cc1 ; Cc1ccc(S(=O)(=O)O)cc1]

p-Toluenesulfonic acid monohydrate (577.24 mg, 3.03 mmol) was added to a stirred solution of tert-butyl 3-[4-(2-carbamoyl-4,4-difluoro-1-piperidyl)phenyl]azetidine-1-carboxylate (400.0 mg, 1.01 mmol) in ACN (20 mL). The reaction mixture was refluxed for 6 h, before being cooled down to 23° C. The resulting precipitate was collected by filtration and recrystallized from i-PrOH, to give the title compound (378.4 mg, 58.48% yield) as a white solid. MS (ESI): m/z=296.2 [M+H]⁺

### Step a): tert-butyl 3-(4-bromophenyl)azetidine-1-carboxylate

To a mixture of 4-bromophenylboronic acid (CAS RN: 5467-74-3; 200.04 g, 996.08 mmol) and tert-butyl 3-iodoazetidine-1-carboxylate (CAS RN: 254454-54-1; 141.0 g, 498.04 mmol) in 2-propanol (500 mL) was added rac-(1R,2R)-2-aminocyclohexan-1-ol (3.44 g, 29.88 mmol), and nickel(II) iodide (9.34 g, 29.88 mmol). A mixture of sodium bis(trimethylsilyl)amide in THF (1 L, 1000 mmol) was added slowly to the reaction mixture, under N₂ and keeping the temperature below 30° C. After the resulting mixture was stirred at 25° C. for 30 min, the mixture was heated to 80° C. and stirred for 12 h. The reaction mixture was poured onto H₂O (3 L) and EtOAc(3 L), and the layers were separated. The aqueous layer was extracted twice with EtOAc (2×2 L). The combined organic layers were evaporated, and purified by FC (SiO₂; PE/EtOAc), to give the title compound (140 g, 90.04% yield) as an off-white oil. MS (ESI): m/z=256.1 [M−tBu+H]⁺

### Step b): 1-[4-(1-tert-butoxycarbonylazetidin-3-yl)phenyl]-4,4-difluoro-piperidine-2-carboxylic acid

A mixture of tert-butyl 3-(4-bromophenyl)azetidine-1-carboxylate (2.5 g, 8.01 mmol), 4,4-difluoropiperidine-2-carboxylic acid;hydrochloride (4.04 g, 20.02 mmol) copper(I) iodide (0.05 mL, 1.6 mmol), phosphoric acid, potassium salt (3.31 mL, 40.04 mmol) and potassium carbonate (2213.42 mg, 16.02 mmol) in DMSO (40 mL) was stirred at 110° C. for 24 h (sealed tube, Argon), before being cooled down to 23° C. The mixture was poured into water and acidified with an aqueous citric acid solution, and extracted with EtOAc (3×25 mL). The organic layers were combined, washed with brine, and evaporated. Purification by FC (SiO₂; CHCl₃/ACN) gave the title compound (700.0 mg, 21.61% yield) as a white solid. MS (ESI): m/z=395.2 [M−H]⁻

### Step c): tert-butyl 3-[4-(2-carbamoyl-4,4-difluoro-]-piperidyl)phenyl]azetidine-]-carboxylate

A solution of 1-[4-(1-tert-butoxycarbonylazetidin-3-yl)phenyl]-4,4-difluoro-piperidine-2-carboxylic acid (400.0 mg, 1.01 mmol) and N,N′-carbonyldiimidazole (212.69 mg, 1.31 mmol) in THF (20 mL) was stirred at 50° C. for 1 h, before being cooled down and treated with ammonia (28% in water) (171.83 mg, 10.09 mmol). The mixture was stirred for another 12 h at 23° C., before being evaporated. The residue was treated with water, and the resulting precipitate was filtered and dried, to give the title compound (400.0 mg, 95.24% yield) as a white solid. MS (ESI): m/z=394.2 [M−H]⁻

### Example E.1

### 6-[(4-dimethylphosphorylphenyl)methyl]-2-azaspiro[3.3]heptane;4-methylbenzenesulfonic acid

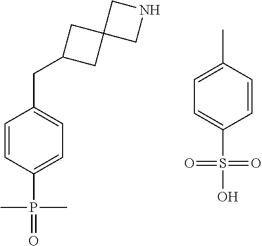 [SMILES: CP(C)(=O)c1ccc(CC2CC3(CNC3)C2)cc1 ; Cc1ccc(S(=O)(=O)O)cc1]

To a solution of tert-butyl 6-[(4-dimethylphosphorylphenyl)methyl]-2-azaspiro[3.3]heptane-2-carboxylate (1.35 g, 3.71 mmol) in EtOAc (50 mL), p-toluenesulfonic acid monohydrate (1.41 g, 7.43 mmol) was added. The mixture was stirred for 12 h at 25° C., before being evaporated to dryness. The residue was purified by RP-HPLC, to give the title compound (474.3 mg, 27.85% yield) as a light yellow solid. MS (ESI): m/z=264.2 [M+H]⁺

### Step a) tert-butyl 6-[(4,4,5,5-tetramethyl-1, 3,2-dioxaborolan-2-yl)methylene]-2-azaspiro[3.3]heptane-2-carboxylate

A mixture of 2,2,6,6-tetramethylpiperidine (95.9 mL, 568 mmol) in THF (750 mL) was cooled to −30° C. under a N₂ atmosphere. n-BuLi (227 mL, 568 mmol) was added dropwise, and the reaction mixture was stirred at the same temperature for 30 min. Next, the reaction was cooled to −60° C., and a solution of 4,4,5,5-tetramethyl-2-[(4,4,5,5-tetramethyl-1,3,2-dioxaborolan-2-yl)methyl]-1,3,2-dioxaborolane (136 g, 506 mmol) in THF (750 mL) was added dropwise. After stirring for 30 min, a solution of tert-butyl 6-oxo-2-azaspiro[3.3]heptane-2-carboxylate (100 g, 473 mmol) in THF (300 mL) was added in dropwise at −60° C. The reaction mixture was allowed to slowly warm up to 25° C. and stirred at 25° C. for 12 h. The mixture was added H₂O (80 mL) slowly and then purified together with an additional batch of equal size by silica gel column (PE/EA=1:0 to 3:1 gradient) to give the title compound (220 g, 656 mmol, approx 69% yield per batch) as a white solid which was confirmed by ¹H NMR (400 MHz, CHLOROFORM-d) δ=5.21-5.16 (m, 1H), 3.99-3.89 (m, 4H), 3.13-2.90 (m, 4H), 1.46-1.41 (m, 9H), 1.26-1.20 ppm (m, 13H).

### Step b) tert-butyl 6-[(4-dimethylphosphorylphenyl)methylene]-2-azaspiro[3.3]heptane-2-carboxylate

To a stirred suspension of 1-bromo-4-dimethylphosphoryl-benzene (1.75 g, 7.52 mmol), tert-butyl 6-[(4,4,5,5-tetramethyl-1,3,2-dioxaborolan-2-yl)methylene]-2-azaspiro[3.3]heptane-2-carboxylate (2.52 g, 7.52 mmol) and potassium carbonate (2.08 g, 15.03 mmol) in 1,4-Dioxane (59.5 mL) and Water (10.5 mL), flushed with Argon for 5 minutes, Pd(dppf)Cl₂—CH₂Cl₂ (1043.53 mg, 1.28 mmol) was added. The mixture was stirred for 18 h at 80° C. in Argon atmosphere (sealed tube). After cooling to RT, the reaction mixture was filtered through SiO₂ (10 g) and washed with 1,4-dioxane (50 mL). The filtrate was concentrated to give crude product which was purified by FC (SiO₂; PE/MTBE then MTBE/MeOH) to obtain the title compound (1.40 g, 46.9% yield) as a grey solid. MS (ESI): m/z=362.2 [M+H]⁺

### Step c) tert-butyl 6-[(4-dimethylphosphorylphenyl)methyl]-2-azaspiro[3.3]heptane-2-carboxylate

The stirred solution of tert-butyl 6-[(4-dimethylphosphorylphenyl)methylene]-2-azaspiro[3.3]heptane-2-carboxylate (1.40 g, 3.87 mmol) and Pd/C (10%) (140 mg) in EtOAc (100 mL) was hydrogenated at 3800 mmHg for 18 h at 25° C. The reaction mixture was filtered and concentrated in vacuum to give the crude title compound (1.35 g, 79.59% yield) as a light green solid. MS (ESI): m/z=364.4 [M+H]⁺

### Example E.2

### 6-[(5-dimethylphosphoryl-2-pyridyl)methyl]-2-azaspiro[3.3]heptane;4-methylbenzenesulfonic acid

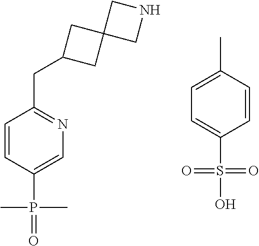 [SMILES: CP(C)(=O)c1ccc(CC2CC3(CNC3)C2)nc1 ; Cc1ccc(S(=O)(=O)O)cc1]

A solution of tert-butyl 6-[(5-dimethylphosphoryl-2-pyridyl)methyl]-2-azaspiro[3.3]heptane-2-carboxylate (998.0 mg, 2.74 mmol) and p-toluenesulfonic acid monohydrate (1.30 g, 6.85 mmol) in EtOAc (70 mL) was stirred at 25° C. for 18 h, before being evaporated. Trituration with MTBE and Et₂O gave the title compound (781.0 mg, 44.51% yield) as a light brown waxy solid. MS (ESI): m/z=265.2 [M+H]⁺

### Step a) tert-butyl 6-[(4,4,5,5-tetramethyl-1, 3,2-dioxaborolan-2-yl)methylene]-2-azaspiro[3.3]heptane-2-carboxylate

A mixture of 2,2,6,6-tetramethylpiperidine (95.9 mL, 568 mmol) in THF (750 mL) was cooled to −30° C. under a N₂ atmosphere. n-BuLi (227 mL, 568 mmol) was added dropwise, and the reaction mixture was stirred at the same temperature for 30 min. Next, the reaction was cooled to −60° C., and a solution of 4,4,5,5-tetramethyl-2-[(4,4,5,5-tetramethyl-1,3,2-dioxaborolan-2-yl)methyl]-1,3,2-dioxaborolane (136 g, 506 mmol) in THF (750 mL) was added dropwise. After stirring for 30 min, a solution of tert-butyl 6-oxo-2-azaspiro[3.3]heptane-2-carboxylate (100 g, 473 mmol) in THF (300 mL) was added in dropwise at −60° C. The reaction mixture was allowed to slowly warm up to 25° C. and stirred at 25° C. for 12 h. The mixture was added H₂O (80 mL) slowly and then purified together with an additional batch of equal size by silica gel column (PE/EA=1:0 to 3:1 gradient) to give the title compound (220 g, 656 mmol, approx 69% yield per batch) as a white solid which was confirmed by ¹H NMR (400 MHz, CHLOROFORM-d) δ=5.21-5.16 (m, 1H), 3.99-3.89 (m, 4H), 3.13-2.90 (m, 4H), 1.46-1.41 (m, 9H), 1.26-1.20 ppm (m, 13H).

### Step b) tert-butyl 6-[(5-dimethylphosphoryl-2-pyridyl)methylene]-2-azaspiro[3.3]heptane-2-carboxylate

2-Chloro-5-dimethylphosphoryl-pyridine (1.02 g, 5.37 mmol), tert-butyl 6-[(4,4,5,5-tetramethyl-1,3,2-dioxaborolan-2-yl)methylene]-2-azaspiro[3.3]heptane-2-carboxylate (1.80 g, 5.37 mmol), Pd(dppf)Cl₂·CH₂Cl₂ (657.69 mg, 0.81 mmol) and potassium carbonate (1.48 g, 10.74 mmol) were dissolved in 1,4-Dioxane (50 mL) and Water (10 mL). The reaction mixture was stirred at 88° C. under Argon for 8 h (sealed tube). The reaction mixture was evaporated. The residue was partitioned between ethyl acetate and water. The organic layer was washed with brine. The organic layer was dried over sodium sulfate, filtered and evaporated. Purification by FC (SiO₂; PE/MTBE) gave the title compound (1.10 g, 51.44% yield) as a yellow solid. MS (ESI): m/z=363.2 [M+H]⁺

### Step c) tert-butyl 6-[(5-dimethylphosphoryl-2-pyridyl)methyl]-2-azaspiro[3.3]heptane-2-carboxylate

A solution of tert-butyl 6-[(5-dimethylphosphoryl-2-pyridyl)methylene]-2-azaspiro[3.3]heptane-2-carboxylate (1.10 g, 3.04 mmol) and Pd/C (10%) (110 mg) in EtOAc (100 mL) was hydrogenated at 3800 mmHg for 48 h at RT (LCMS control). The reaction mixture was filtered off and evaporated, to give the crude title compound (998.0 mg, 85.71% yield) as a light grey solid. MS (ESI): m/z=365.2 [M+H]⁺

### Example E.3

### 2-[(4-dimethylphosphorylphenyl)methyl]-2,6-diazaspiro[3.3]heptane;4-methylbenzenesulfonic acid

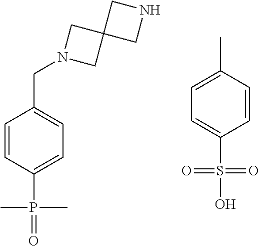 [SMILES: CP(C)(=O)c1ccc(CN2CC3(CNC3)C2)cc1 ; Cc1ccc(S(=O)(=O)O)cc1]

PTSA (73.71 mg, 0.43 mmol) was added to a solution of tert-butyl 6-[(4-dimethylphosphorylphenyl)methyl]-2,6-diazaspiro[3.3]heptane-2-carboxylate (78.0 mg, 0.21 mmol) in EtOAc (20 mL). The reaction mixture was stirred at 50° C. for 16 h. The precipitate was collected by filtration and washed twice with MTBE (2*50 mL), to give the title compound (100.0 mg, 72.92% yield) as a white solid. MS (ESI): m/z=265.2 [M+H]⁺

### Step a) tert-butyl 6-[(4-dimethylphosphorylphenyl)methyl]-2,6-diazaspiro[3.3]heptane-2-carboxylate

To a solution of 1-(bromomethyl)-4-dimethylphosphoryl-benzene (CAS: 2287285-08-7; 200.0 mg, 0.81 mmol) and tert-butyl 2,6-diazaspiro[3.3]heptane-2-carboxylate hydrochloride (CAS 1207840-19-4; 190.0 mg, 0.81 mmol) in ACN (15 mL) was added N,N-diisopropylethylamine (0.42 mL, 2.43 mmol) and stirred at RT overnight. The reaction mixture was then concentrated under vacuum and diluted with EtOAc (15 mL). The organic layer was washed with brine (15 mL), dried over Na₂SO₄, filtered and evaporated. Purification by RP-HPLC gave the title compound (76.0 mg, 24.22% yield) as a colorless oil. MS (ESI): m/z=365.2 [M+H]⁺

### Example E.4

### 3-[(4-dimethylphosphorylphenyl)methoxy]azetidine;4-methylbenzenesulfonic acid

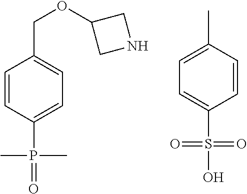 [SMILES: CP(C)(=O)c1ccc(COC2CNC2)cc1 ; Cc1ccc(S(=O)(=O)O)cc1]

A solution of tert-butyl 3-[(4-dimethylphosphorylphenyl)methoxy]azetidine-1-carboxylate (1.25 g, 3.68 mmol) in MeOH (10 mL) was treated with p-toluenesulfonic acid (1585.66 mg, 9.21 mmol), at 23° C. The mixture was stirred for 42 h at this temperature, before being evaporated. Trituration with acetonitrile (20 mL) gave the title compound (1.73 g, 80.38% yield) as a white powder. MS (ESI): m/z=240.0 [M+H]⁺

### Step a) tert-butyl 3-[(4-dimethylphosphorylphenyl)methoxy]azetidine-1-carboxylate

A mixture of tert-butyl 3-hydroxyazetidine-1-carboxylate (725.59 mg, 4.19 mmol), 1-(bromomethyl)-4-dimethylphosphoryl-benzene (CAS 2287285-08-7; 1.15 g, 4.19 mmol) and sodium hydride in mineral oil 60% (251.35 mg, 6.28 mmol) in THF (50 mL) was stirred for 16 h at room temperature. Water (5 mL) was added, and the organic layer was collected and evaporated. Purification by RP-HPLC gave the title compound (1.25 g, 87.93% yield). MS (ESI): m/z=284.2 [M-Bu+H]⁺

### Example E.5

### 3-[3-(azetidin-3-yl)-1-bicyclo[1.1.1]pentanyl]-5-cyclopropyl-4H-1,2,4-triazole;4-methylbenzenesulfonic acid

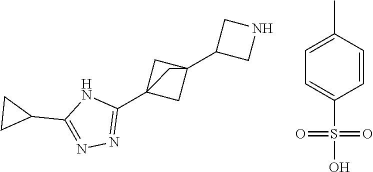 [SMILES: C1CC1c1nnc(C23CC(C4CNC4)(C2)C3)[nH]1 ; Cc1ccc(S(=O)(=O)O)cc1]

A mixture of p-toluenesulfonic acid (1.31 g, 7.63 mmol) and tert-butyl 3-[3-(5-cyclopropyl-4H-1,2,4-triazol-3-yl)-1-bicyclo[1.1.1]pentanyl]azetidine-1-carboxylate (1.2 g, 3.63 mmol) in EtOAc (15 mL) was stirred at 25° C. for 24 h, before being evaporated. Purification by RP-HPLC gave the title compound (840.0 mg, 57.46% yield) as a light yellow solid. MS (ESI): m/z=231.0 [M+H]⁺

### Step a) tert-butyl 3-[3-(hydrazinecarbonyl)-1-bicyclo[1.1.1]pentanyl]azetidine-1-carboxylate

N,N′-carbonyldiimidazole (4.09 g, 25.25 mmol) was added to a solution of 3-(1-tert-butoxycarbonylazetidin-3-yl)bicyclo[1.1.1]pentane-1-carboxylic acid (4.5 g, 16.83 mmol) in THF (85 mL). The reaction mixture was stirred for 1 h at 50° C., before being cooled down to 23° C. Hydrazine (5.4 g, 168.34 mmol) was added to the mixture, which was stirred for 12 h at 23° C. before being evaporated. The residue was dissolved in DCM and washed by water (twice). The organic layer was dried over Na₂SO₄, filtered, and evaporated, to give the title compound (4.5 g, 90.26% yield) as a white solid. MS (ESI): m/z=282.2 [M+H]⁺

### Step b) tert-butyl 3-[3-(5-cyclopropyl-4H-1,2,4-triazol-3-yl)-1-bicyclo[1.1.1]pentanyl]azetidine-1-carboxylate

tert-butyl 3-[3-(hydrazinecarbonyl)-1-bicyclo[1.1.1]pentanyl]azetidine-1-carboxylate (4.5 g, 15.99 mmol, and cyclopropanecarboximidamide hydrochloride (2.7 g, 22.39 mmol) were dissolved in triethylamine (61.36 mL, 440.26 mmol) and pyridine (65 mL). The reaction mixture was degassed with Ar and then heated to 90° C. for 24 h, before being evaporated.

The mixture was diluted with DCM and water. The organic layer was washed with brine (twice) and dried over sodium sulfate, filtered and evaporated. Purification by FC (SiO₂; CHCl₃/ACN) gave the title compound (78 mg, 30.19% yield) as a white solid. MS (ESI): m/z 10=331.2 [M-Bu+H]⁺

### Example E.6

### 3-[3-(azetidin-3-yl)-1-bicyclo[1.1.1]pentanyl]-5-[1-(trifluoromethyl)cyclopropyl]-4H-1,2,4-triazole;4-methylbenzenesulfonic acid

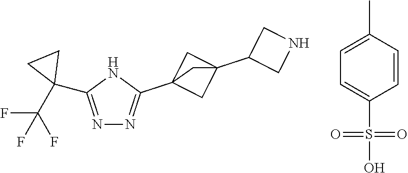 [SMILES: FC(F)(F)C1(c2nnc(C34CC(C5CNC5)(C3)C4)[nH]2)CC1 ; Cc1ccc(S(=O)(=O)O)cc1]

A mixture of p-toluenesulfonic acid (0.1 g, 0.58 mmol) and tert-butyl 3-[3-[5-[1-(trifluoromethyl)cyclopropyl]-4H-1,2,4-triazol-3-yl]-1-bicyclo[1.1.1]pentanyl]azetidine-1-carboxylate (0.1 g, 0.25 mmol) in EtOAc (3 mL) was stirred at 25° C. for 48 h, before being evaporated, to give the title compound (200.0 mg, 84.68% yield) as a colorless oil. MS (ESI): m/z=299.0 [M+H]⁺

### Step a)]-(trifluoromethyl)cyclopropanecarboxamidine;hydrochloride

1-(trifluoromethyl)cyclopropanecarbonitrile (4.0 g, 29.61 mmol) was dissolved in EtOH (30 mL). The reaction mixture was cooled to 10° C., and HCl (Gas) was bubbled through the reaction mixture for 10 min. The mixture was stirred for 12 h at 23° C., before being evaporated. The residue was dissolved in 10 mL EtOH and the mixture was added to 30 mL of a saturated solution of ammonia in EtOH. The mixture was stirred for 12 h at 23° C. The resulting precipitate was filtered off, and the filtrate was evaporated, to give the title compound (2.2 g, 35.46% yield) as a white solid. MS (ESI): m/z=153.0 [M+H]⁺

### Step b) tert-butyl 3-[3-(hydrazinecarbonyl)-1-bicyclo[1.]0.1]pentanyl]azetidine-1-carboxylate

N,N′-carbonyldiimidazole (4.09 g, 25.25 mmol) was added to a solution of 3-(1-tert-butoxycarbonylazetidin-3-yl)bicyclo[1.1.1]pentane-1-carboxylic acid (4.5 g, 16.83 mmol) in THF (85 mL). The reaction mixture was stirred for 1 h at 50° C., before being cooled down to 23° C. Hydrazine (5.4 g, 168.34 mmol) was added to the mixture, which was stirred for 12 h at 23° C. before being evaporated. The residue was dissolved in DCM and washed by water (twice). The organic layer was dried over Na₂SO₄, filtered, and evaporated, to give the title compound (4.5 g, 90.26% yield) as a white solid. MS (ESI): m/z=282.2 [M+H]⁺

### Step c) tert-butyl 3-[3-[5-[ ]-(trifluoromethyl)cyclopropyl]-4H-1,2,4-triazol-3-yl]-1-bicyclo[1.1.1]pentanyl]azetidine-1-carboxylate

tert-butyl 3-[3-(hydrazinecarbonyl)-1-bicyclo[1.1.1]pentanyl]azetidine-1-carboxylate (0.65 g, 2.31 mmol,) and 1-(trifluoromethyl)cyclopropanecarboxamidine;hydrochloride (0.44 g, 2.31 mmol) were dissolved in triethylamine (10.0 mL, 71.75 mmol) and pyridine (10 mL). The reaction mixture was degassed with argon and then heated to 90° C. for 24 h. The reaction mixture was concentrated and diluted between DCM and water. The organic layer was washed with brine (twice) and dried over sodium sulfate, filtered and evaporated to give the title compound (0.8 g, 19.12% yield) as a light yellow oil. MS (ESI): m/z=399.2 [M+H]⁺

### Example E.7

### 3-[[3-(azetidin-3-yl)-1-bicyclo[1.1.1]pentanyl]methyl]-5-cyclopropyl-4H-1,2,4-triazole;2,2,2-trifluoroacetic acid

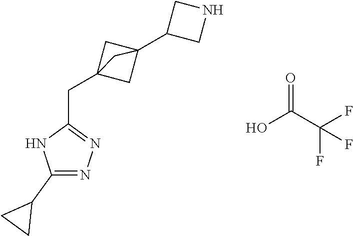 [SMILES: C1CC1c1nnc(CC23CC(C4CNC4)(C2)C3)[nH]1 ; O=C(O)C(F)(F)F]

To a stirred solution of tert-butyl 3-[3-[(5-cyclopropyl-4H-1,2,4-triazol-3-yl)methyl]-1-bicyclo[1.1.1]pentanyl]azetidine-1-carboxylate (70.0 mg, 0.2 mmol) in DCM (3 mL), trifluoroacetic acid (0.08 mL, 1.02 mmol) was added. The mixture was stirred for 18 h at 25° C., before being evaporated. Purification by RP-HPLC gave the title compound (30.3 mg, 29.98% yield) as a light yellow viscous oil. MS (ESI): m/z=245.2 [M+H]⁺

### Step a) tert-butyl 3-(3-isobutoxycarbonyloxycarbonyl-1-bicyclo[1.1.1]pentanyl)azetidine-]-carboxylate

To a stirred solution of 3-(1-tert-butoxycarbonylazetidin-3-yl)bicyclo[1.1.1]pentane-1-carboxylic acid (5.0 g, 18.7 mmol) and 4-methylmorpholine (2.27 g, 22.45 mmol) in THF (150 mL), isobutyl chloroformate (2.55 g, 18.7 mmol) was added dropwise at 0° C. The mixture was stirred for 1 h at 25° C. The resulting precipitate was filtered off and the filtrate was evaporated, to give the title compound (6.1 g, 84.32% yield), which was directly used in the next step.

### Step b) tert-butyl 3-[3-(2-diazoacetyl)-1-bicyclo[1.1.1]pentanyl]azetidine-1-carboxylate

To a stirred solution of tert-butyl 3-(3-isobutoxycarbonyloxycarbonyl-1-bicyclo[1.1.1]pentanyl)azetidine-1-carboxylate (1400.0 mg, 3.81 mmol) in THF (30 mL) was added a solution of diazomethane (480.54 mg, 11.43 mmol) in TBME (50 mL). An ethereal solution of diazomethane (about 125 mL) was then added through the funnel, stirring was resumed for about 5 seconds and the nitrogen stream was stopped. After 45 min, the remainder of the diazomethane solution (about 85 mL) was added. The cooling bath was removed and the solution allowed to react for 3 h, without stirring. Then, 75 mL of 0.5 N acetic acid was added carefully to destroy unreacted diazomethane and saturated aqueous sodium bicarbonate solution (75 mL) was added carefully. The aqueous layer was separated in a separatory funnel and the organic layer was washed with saturated aqueous sodium chloride (75 mL). The organic layer was dried over magnesium sulfate, filtered, and evaporated. The crude product was placed under high vacuum for 3 h, and was then directly used in the next step.

Caution! Diazomethane should be handled in an efficient fume hood behind a protection shield because of its toxicity and the possibility of explosions.

### Step c) 2-[3-(1-tert-butoxycarbonylazetidin-3-yl)-1-bicyclo[1.1.1]pentanyl]acetic acid

A 500-mL, three-necked flask was equipped with a nitrogen gas inlet, bubble counter, septum and a magnetic stirring bar. The flask was carefully wrapped in aluminum foil (to exclude light during the reaction). The crude diazo ketone from the preceding step was dissolved in tetrahydrofuran (380 mL) and added to the flask under nitrogen. De-ionized water (38 mL) was added, the flask was immersed in a dry ice-acetone bath, and the solution is cooled to −25° C. (temperature of the acetone cooling bath) for 30 min. Silver trifluoroacetate (2.72 g, 12.3 mmol) was placed in a 50-mL Erlenmeyer flask and quickly dissolved in triethylamine (39 mL, 279 mmol). The resulting solution was added to the diazoketone solution in one portion (via syringe). The solution was allowed to warm to room temperature overnight. Evolution of nitrogen started at a bath temperature of about −15° C. The solution was transferred to a 1-L, round-bottomed flask and the reaction vessel was rinsed with ethyl acetate (2×10 mL). The solution was evaporated to dryness with a rotary evaporator and the residue was stirred for 1 h with saturated aqueous sodium bicarbonate (NaHCO₃) solution (100 mL). The black mixture was transferred into a 1-L separatory funnel with water (150 mL) and ethyl acetate (200 mL), and the mixture was shaken well. The clear aqueous layer was separated and put aside, leaving an organic phase containing a suspension of black solid. Brine (30 mL) was added to the organic phase and the resulting mixture was shaken vigorously. Saturated, aqueous NaHCO₃ solution (30 mL) is added, the medium is shaken again, and the layers are separated. The black solid was carried away with the aqueous phase, which was now combined with the first-separated aqueous phase. The organic layer was washed with three additional portions of saturated aqueous NaHCO₃ solution (30 mL each) and all the aqueous layers were combined. The first organic layer was put aside and not used further. The combined aqueous layers containing a black suspension were extracted with ethyl acetate (50 mL) and the ethyl acetate layer was then back-extracted with two portions of saturated aqueous NaHCO₃ solution (25 mL each), which were combined with the original aqueous layers. The ethyl acetate was put aside and not used further. All the combined aqueous layers were extracted again with 50 mL of ethyl acetate, which was washed with saturated aqueous NaHCO₃ solution (2×20 mL). The organic layer was put aside and not used further. All the combined aqueous layers are then transferred to a 2-L, round-bottomed flask equipped with a magnetic stirring bar and about 10 drops of Congo Red indicator and ethyl acetate (100 mL) were added. The organic layer was dried over Na₂SO₄, filtered, and evaporated, to give the title compound (3.20 g, 71.6% yield) as a light yellow solid. MS (ESI): m/z=280.1 [M−H]⁻

### Step d) tert-butyl 3-[3-(2-hydrazino-2-oxo-ethyl)-]-bicyclo[ ]_{0.1.1}]pentanyl]azetidine-]-carboxylate

To a stirred solution of 2-[3-(1-tert-butoxycarbonylazetidin-3-yl)-1-bicyclo[1.1.1]pentanyl]acetic acid (1180.0 mg, 4.19 mmol) in THF (50 mL), N,N′-carbonyldiimidazole (1020.1 mg, 6.29 mmol) was added. The mixture was stirred for 45 min at 50° C., before being cooled down to 25° C. Hydrazine hydrate (2.1 mL, 41.94 mmol) was added, and the mixture was stirred for 18 h at 25° C., before being evaporated. The residue was partioned between DCM (100 mL) and water (100 mL). The water layer was washed with DCM (2×50 mL). The combined organic layers were washed with brine, dried over Na₂SO₄, filtered, and evaporated, to give the title compound (1.20 g, 88.15% yield) as a yellow viscous oil. MS (ESI): m/z=294.2 [M−H]⁻

### Step e) tert-butyl 3-[3-[(5-cyclopropyl-4H-1,2,4-triazol-3-yl)methyl]-1-bicyclo[1.1.1]pentanyl]azetidine-1-carboxylate

A solution of tert-butyl 3-[3-(2-hydrazino-2-oxo-ethyl)-1-bicyclo[1.1.1]pentanyl]azetidine-1-carboxylate (1200.0 mg, 4.06 mmol), cyclopropanecarboximidamide hydrochloride (832.77 mg, 6.91 mmol), triethylamine (18.0 mL, 129.14 mmol) and pyridine (18.0 mL, 222.55 mmol) was stirred at 90° C. for 18 h, before being evaporated and partitioned between water and CHCl₃. The organic layer was dried over Na₂SO₄, filtered, and evaporated. Purification by RP-HPLC gave the title compound (72.0 mg, 4.89% yield) as a light yellow oil. MS (ESI): m/z=345.2 [M+H]⁺

### Example E.8

### 1-[3-(azetidin-3-yl)-1-bicyclo[1.1.1]pentanyl]-5-cyclopropyl-3-methyl-pyrazole;4-methylbenzenesulfonic acid

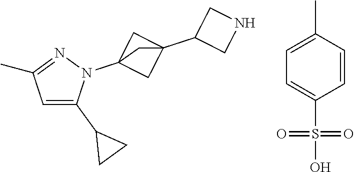 [SMILES: Cc1cc(C2CC2)n(C23CC(C4CNC4)(C2)C3)n1 ; Cc1ccc(S(=O)(=O)O)cc1]

To a solution of tert-butyl 3-[3-(5-cyclopropyl-3-methyl-pyrazol-1-yl)-1-bicyclo[0.1.1]pentanyl]azetidine-1-carboxylate (558.0 mg, 1.62 mmol) in MeOH (3 mL), p-toluenesulfonic acid (419.65 mg, 2.44 mmol) was added, and the resulting mixture was stirred for 16 h. The mixture was evaporated, triturated with acetonitrile (10 mL), filtered and dried. Purification by RP-HPLC gave the title compound (390.0 mg, 54.88% yield) as a white solid. MS (ESI): m/z=244.0 [M+H]⁺

### Step a) tert-butyl 3-[3-(benzyloxycarbonylamino)-1-bicyclo[1.]0.1]pentanyl]azetidine-]-carboxylate

To a solution of 3-(1-tert-butoxycarbonylazetidin-3-yl)bicyclo[1.1.1]pentane-1-carboxylic acid (4.25 g, 15.9 mmol) and benzyl alcohol (3.29 mL, 31.8 mmol) in Toluene (60 mL) at ambient temperature was added triethylamine (6.65 mL, 47.7 mmol). The mixture was stirred for 5 min, and diphenylphosphonic azide (3.6 mL, 16.69 mmol) was added. The mixture was stirred another 15 min at ambient temperature, and for 16 h at 100° C. After cooling, the mixture was poured into ice-cold water (100 mL) and extracted with MTBE. The organic layer was washed with H₂O and brine, dried over Na₂SO₄, filtered, and evaporated. Purification by FC gave the title compound (3.7 g, 59.36% yield) as a white solid. MS (ESI): m/z=371.2 [M−H]⁻

### Step b): tert-butyl 3-(3-amino-]-bicyclo[1.1.1]pentanyl)azetidine-1-carboxylate

To a solution of tert-butyl 3-[3-(benzyloxycarbonylamino)-1-bicyclo[1.1.1]pentanyl]azetidine-1-carboxylate (4.15 g, 11.14 mmol) in MeOH (50 mL) was added Pd/C (10%) (0.58 mL, 0.56 mmol). The reaction mixture was stirred for 48 h at room temperature under hydrogen atmosphere. The solids were removed by filtration and the filtrate was concentrated in vacuo, to give the title compound (2.6 g, 93.01% yield) as a colorless oil. MS (ESI): m/z=239.2 M+H]+

### Step c): tert-butyl 3-[3-(5-cyclopropyl-3-methyl-pyrazol-1-yl)-1-bicyclo[1.1.1]pentanyl]azetidine-1-carboxylate

To a solution of tert-butyl 3-(3-amino-1-bicyclo[1.1.1]pentanyl)azetidine-1-carboxylate (1.3 g, 5.45 mmol) in DMF (10 mL) 1-cyclopropylbutane-1,3-dione (0.76 g, 6.0 mmol) and O-(4-nitrobenzoyl)hydroxylamine (1.49 g, 8.18 mmol) were added. The mixture was stirred at 85° C. for 2 h, cooled down, water (25 mL) was added and extracted with at EtOAc (3 times 15 mL each). The organics were then separated and dried (Na₂SO₄) before concentration to dryness. Purification by RP-HPLC gave the title compound (558.0 mg, 29.78% yield). MS (ESI): m/z=344.2 [M+H]⁺ Example 540

A compound of formula (I) can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example 541

A compound of formula (I) can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1. A compound of formula (I) 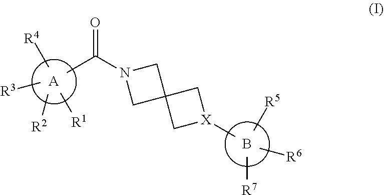
or a pharmaceutically acceptable salt thereof, wherein:
X is CR⁸ or N;
A is selected from 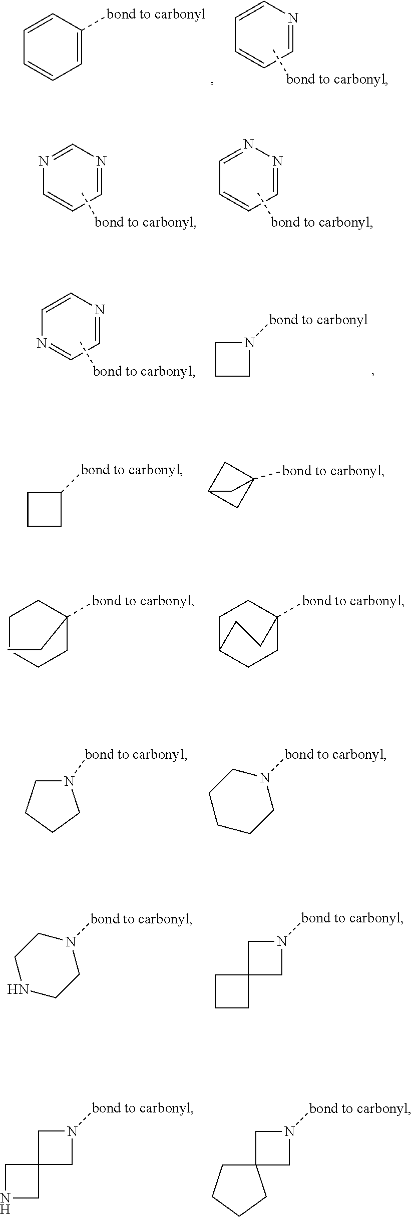 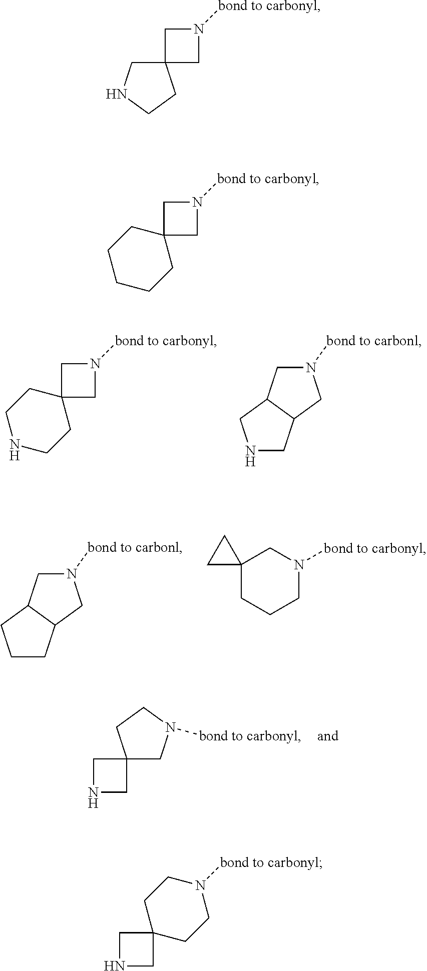
B is a heteroaryl selected from B-1 to B-10: 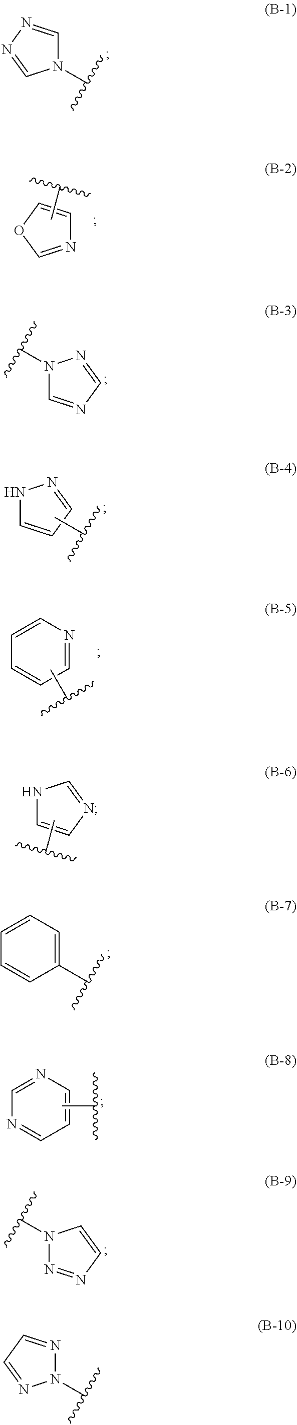
wherein the wavy line indicates the point of attachment to the remainder of formula (I);
C is selected from C₆-C₁₄-aryl, C₃-C₁₀-cycloalkyl, 5- to 6-membered heteroaryl, and 3- to 14-membered heterocyclyl;
D is selected from C₃-C₁₀-cycloalkyl, 3- to 14-membered heterocyclyl, C₆-C₁₄-aryl, and 5- to 14-membered heteroaryl;
E is selected from C₃-C₁₀-cycloalkyl, and 3- to 14-membered heterocyclyl;
L¹ is selected from a covalent bond, —CR¹²R¹³—, —CH₂O—, —OCH₂—, —CH₂NH—, —NHCH₂—, —CH₂OCH₂—, —O—, —NH—, —CH₂CH₂—, —CH═CH—, —SO₂NH—, —NHSO₂—, —SO₂NHCH₂—, —CH₂NHSO₂—, —SO₂—, —CH₂SO₂—, —SO₂CH₂—, —(CH₂)₂SO₂—, —SO₂(CH₂)₂—, carbonyl, —NHC(O)— and —C(O)NH—;
L² is selected from a covalent bond, —CH₂—, —CH₂NH—, —NHCH₂—, —NH—, —N(C₁-C₆-alkyl)- and —SO₂—;
L³ is selected from a covalent bond and —CH₂—; 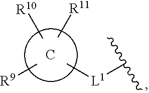
R¹ is selected from hydrogen, halogen, a group L, C₁-C₆-alkyl, halo-C₁-C₆-alkyl, C₁-C₆-alkoxy, halo-C₁-C₆-alkoxy, C₁-C₆-alkyl-SO₂NH—, C₃-C₁₀-cycloalkyl-C₁-C₆-alkyl-S(O)₂—, C₁—C-alkyl-SO₂—, halo-C₁-C₆-alkyl-S(O)₂—, (C₁-C₆-alkyl)₂N—SO₂—, and halo-C₁-C₆-alkyl-C(O)—;
R², R³, and R⁴ are each independently selected from hydrogen, halogen, C₁-C₆-alkyl, halo-C₁-C₆-alkyl, and 3- to 14-membered heterocyclyl;
R⁵ and R⁶ are each independently selected from hydrogen, halogen, cyano, hydroxy, amino, C₁-C₆-alkyl, C₁-C₆-alkoxy, halo-C₁-C₆-alkyl, halo-C₁-C₆-alkoxy, C₃-C₁₀-cycloalkyl, and 3- to 14-membered heterocyclyl; wherein said C₃-C₁₀-cycloalkyl and 3- to 14-membered heterocyclyl are optionally substituted with 1, 2 or 3 substituents selected from C₁-C₆-alkyl, halo-C₁-C₆-alkyl, C₁-C₆-alkoxy, halo-C₁-C₆-alkoxy, halogen, cyano, amino, and hydroxy;
R⁷ is absent or is selected from hydrogen, halogen, cyano, hydroxy, amino, C₁-C₆-alkyl, C₁-C₆-alkoxy, halo-C₁-C₆-alkyl, and halo-C₁-C₆-alkoxy;
R⁸ is selected from hydrogen, halogen, cyano, C₁-C₆-alkyl, C₁-C₆-alkoxy, and hydroxy;
R⁹ is selected from hydrogen, C₁-C₆-alkyl, C₁-C₆-alkoxy, halo-C₁-C₆-alkyl, halo-C₁-C₆-alkoxy, halogen, cyano, SF₅, C₁—C-alkyl-SO₂—, halo-C₁-C₆-alkyl-SO₂—, (C₁-C₆-alkyl)₂-PO—, amino, carboxy, carboxy-C₁-C₆-alkyl, C₁-C₆-alkoxycarbonyl, C₁-C₆-alkoxycarbonyl-C₁-C₆-alkyl-, NH₂SO₂—, carbamoyl, C₁-C₆-alkyl-C(O)NH—, halo-C₁-C₆-alkyl-NHC(O)—, oxo, a group 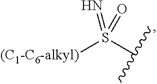
a group 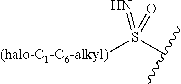
and a group 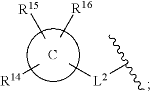
R¹⁰ and R¹¹ are each independently selected from hydrogen, halogen, cyano, C₁-C₆-alkyl, C₁-C₆-alkoxy, halo-C₁-C₆-alkyl, and oxo;
R¹² is selected from hydrogen, carbamoyl, C₁-C₆-alkyl-NHC(O)—, and halo-C₆-C₁₄-aryl and R¹³ is hydrogen; or
R¹² and R¹³, taken together with the carbon atom to which they are attached, form a C₃-C₁₀-cycloalkyl;
R¹⁴ is selected from hydrogen, C₁-C₆-alkyl, halo-C₁-C₆-alkyl, C₁-C₆-alkoxy, halo-C₁-C₆-alkoxy, halogen, cyano, amino, carbamoyl, hydroxy, oxo, C₁—C-alkyl-SO₂—, and a group 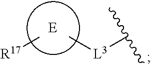
R¹⁵ is selected from hydrogen, halogen, hydroxy, oxo, and C₁-C₆-alkyl;
R¹⁶ is selected from hydrogen and halogen; and
R¹⁷ is selected from hydrogen, C₁-C₆-alkyl, and halo-C₁-C₆-alkyl.

2. The compound of formula (I) according to claim 1, or a pharmaceutically acceptable salt thereof, wherein:
X is CR⁸;
A is selected from 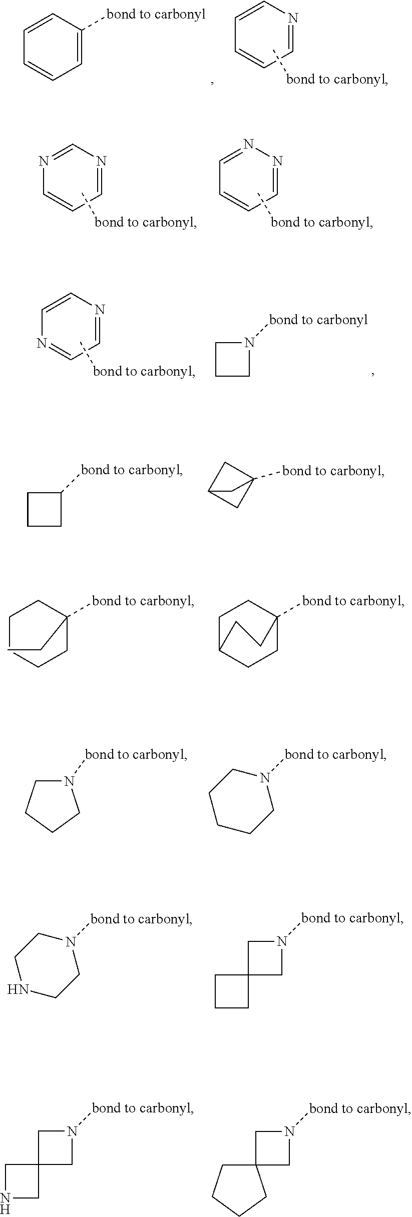 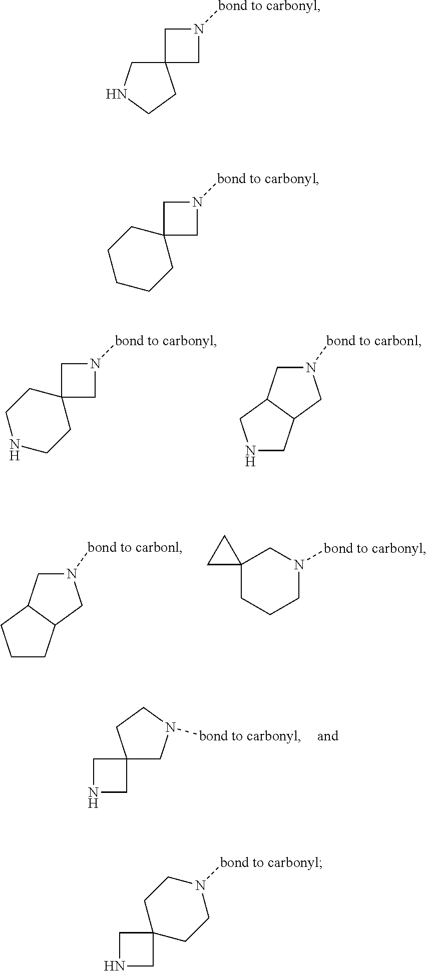
B is a heteroaryl selected from B-1 to B-6: 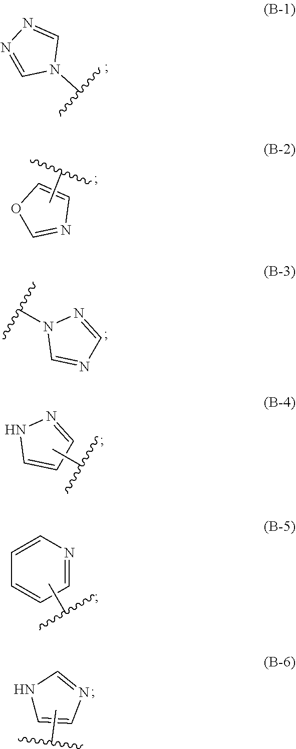
wherein the wavy line indicates the point of attachment to the remainder of formula (I);
C is selected from C₆-C₁₄-aryl, C₃-C₁₀-cycloalkyl, 5- to 6-membered heteroaryl, and 3- to 14-membered heterocyclyl;
L¹ is selected from a covalent bond, —CR¹²R¹³—, —CH₂O—, —OCH₂— —CH₂NH—, —NHCH₂—, —CH₂OCH₂—, —O—, —NH—, [symbol] , —SO₂NH—, —NHSO₂—, —SO₂NHCH₂—, —CH₂NHSO₂—, —SO₂—, —CH₂SO₂—, —SO₂CH₂—, —(CH₂)₂SO₂—, —SO₂(CH₂)₂—, carbonyl, —NHC(O)— and —C(O)NH—;
R¹ is selected from hydrogen, halogen, a group 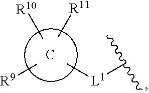
C₁-C₆-alkyl, halo-C₁-C₆-alkyl, C₁-C₆-alkoxy, halo-C₁-C₆-alkoxy, C₁-C₆-alkyl-SO₂NH—, C₃-C₁₀-cycloalkyl-C₁-C₆-alkyl-S(O)₂—, C₁—C-alkyl-SO₂—, halo-C₁-C₆-alkyl-S(O)₂—, (C₁-C₆-alkyl)₂N—SO₂—, and halo-C₁-C₆-alkyl-C(O)—;
R², R³, and R⁴ are each independently selected from hydrogen, halogen, C₁-C₆-alkyl, halo-C₁-C₆-alkyl, and 3- to 14-membered heterocyclyl;
R⁵ and R⁶ are each independently selected from hydrogen, halogen, cyano, hydroxy, amino, C₁-C₆-alkyl, C₁-C₆-alkoxy, halo-C₁-C₆-alkyl, halo-C₁-C₆-alkoxy, C₃-C₁₀-cycloalkyl, and 3- to 14-membered heterocyclyl;
wherein C₃-C₁₀-cycloalkyl and 3- to 14-membered heterocyclyl are optionally substituted with 1, 2 or 3 substituents selected from C₁-C₆-alkyl, halo-C₁-C₆-alkyl, C₁-C₆-alkoxy, halo-C₁-C₆-alkoxy, halogen, cyano, amino, and hydroxy;
R⁷ is absent or is selected from hydrogen, halogen, cyano, hydroxy, amino, C₁-C₆-alkyl, C₁-C₆-alkoxy, halo-C₁-C₆-alkyl, and halo-C₁-C₆-alkoxy;
R⁸ is selected from hydrogen, halogen, cyano, C₁-C₆-alkyl, C₁-C₆-alkoxy, and hydroxy;
R⁹ is selected from hydrogen, C₁-C₆-alkyl, C₁-C₆-alkoxy, halo-C₁-C₆-alkyl, halo-C₁-C₆-alkoxy, halogen, cyano, SF₅, C₃-C₁₀-cycloalkyl, C₃-C₁₀-cycloalkyl-C₁-C₆-alkyl-, 3- to 14-membered heterocyclyl, C₆-C₁₄-aryl, C₁—C-alkyl-SO₂—, amino, carboxy, carboxy-C₁-C₆-alkyl, C₁-C₆-alkoxycarbonyl, C₁-C₆-alkoxycarbonyl-C₁-C₆-alkyl-, NH₂SO₂—, carbamoyl, C₁-C₆-alkyl-C(O)NH—, halo-C₁-C₆-alkyl-NHC(O)— and oxo;
wherein C₃-C₁₀-cycloalkyl, 3- to 14-membered heterocyclyl, and C₆-C₁₄-aryl are optionally substituted with 1, 2, or 3 substituents selected from C₁-C₆-alkyl, halo-C₁-C₆-alkyl, C₁-C₆-alkoxy, halo-C₁-C₆-alkoxy, 3- to 14-membered heterocyclyl, halogen, cyano, amino, and hydroxy;
R¹⁰ and R¹¹ are each independently selected from hydrogen, halogen, cyano, C₁-C₆-alkyl, C₁-C₆-alkoxy, halo-C₁-C₆-alkyl, and oxo;
R¹² is selected from hydrogen, carbamoyl, C₁-C₆-alkyl-NHC(O)—, and halo-C₆-C₁₄-aryl and R¹³ is hydrogen; or
R¹² and R¹³, taken together with the carbon atom to which they are attached, form a C₃-C₁₀-cycloalkyl.

3. The compound of formula (I) according to claim 1, or a pharmaceutically acceptable salt thereof, wherein:
A is selected from 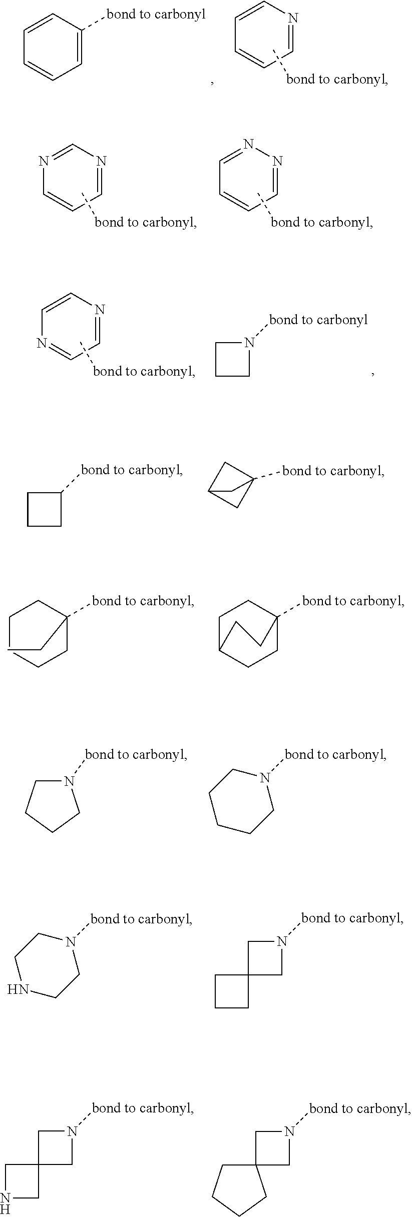 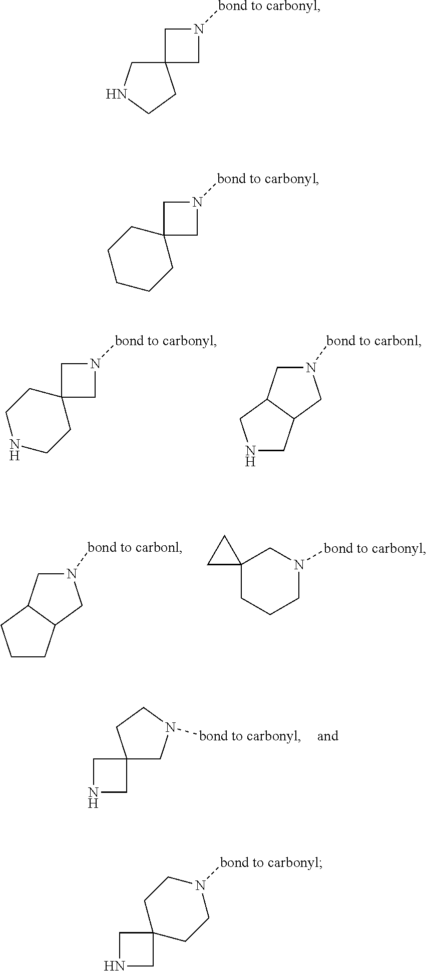
C is selected from C₆-C₁₄-aryl, C₃-C₁₀-cycloalkyl, 5- to 6-membered heteroaryl, and 3- to 14-membered heterocyclyl;
D is selected from C₃-C₁₀-cycloalkyl, 3- to 14-membered heterocyclyl, C₆-C₁₄-aryl and 5- to 14-membered heteroaryl;
E is selected from C₃-C₁₀-cycloalkyl, and 3- to 14-membered heterocyclyl;
L¹ is selected from a covalent bond, —CR¹²R¹³—, —CH₂O—, —CH₂NH—, —CH₂OCH₂—, —O—, —NH—, [symbol] , —SO₂NH—, —NHSO₂—, —SO₂NHCH₂—, —CH₂NHSO₂—, —SO₂—, —CH₂SO₂—, —(CH₂)₂SO₂—, carbonyl, and —C(O)NH—;
L² is selected from a covalent bond, —CH₂—, —CH₂NH—, —NHCH₂—, —NH—, —N(C₁-C₆-alkyl)- and —SO₂—;
L³ is selected from a covalent bond and —CH₂—;
R¹ is selected from a group 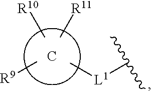
halo-C₁-C₆-alkoxy, C₁-C₆-alkyl-SO₂NH—, C₃-C₁₀-cycloalkyl-C₁-C₆-alkyl-S(O)₂—, C₁—C-alkyl-SO₂—, halo-C₁-C₆-alkyl-S(O)₂—, (C₁-C₆-alkyl)₂N—SO₂—, and halo —C₆-alkyl-C(O)—;
R² is selected from hydrogen, halogen, C₁-C₆-alkyl, halo-C₁-C₆-alkyl, and 3- to 14-membered heterocyclyl;
R³ is selected from hydrogen and halogen;
R⁴ is hydrogen;
R⁹ is selected from hydrogen, C₁-C₆-alkyl, C₁-C₆-alkoxy, halo-C₁-C₆-alkyl, halo-C₁-C₆-alkoxy, halogen, cyano, SF₅, C₁—C-alkyl-SO₂—, halo-C₁—C-alkyl-SO₂—, (C₁-C₆-alkyl)₂-PO—, amino, carboxy, carboxy-C₁-C₆-alkyl, C₁-C₆-alkoxycarbonyl, C₁-C₆-alkoxycarbonyl-C₁-C₆-alkyl-, NH₂SO₂—, carbamoyl, C₁-C₆-alkyl-C(O)NH—, halo-C₁-C₆-alkyl-NHC(O)—, oxo, a group 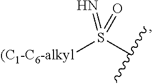
a group 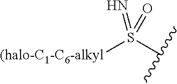
and a group 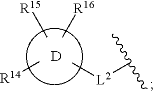
wherein C₃-C₁₀-cycloalkyl, 3- to 14-membered heterocyclyl, and C₆-C₁₄-aryl are optionally substituted with 1 or 2 substituents selected from halo-C₁-C₆-alkyl, 3- to 14-membered heterocyclyl, halogen, and hydroxy;
R¹⁰ is selected from hydrogen, halogen, cyano, C₁-C₆-alkyl, C₁-C₆-alkoxy, halo-C₁-C₆-alkyl, and oxo;
R¹¹ is selected from hydrogen and halogen;
R¹² is selected from hydrogen, carbamoyl, C₁-C₆-alkyl-NHC(O)—, and halo-C₆-C₁₄-aryl and R¹³ is hydrogen; or
R¹² and R³, taken together with the carbon atom to which they are attached, form a C₃-C₁₀-cycloalkyl;
R¹⁴ is selected from hydrogen, C₁-C₆-alkyl, halo-C₁-C₆-alkyl, C₁-C₆-alkoxy, halo-C₁-C₆-alkoxy, halogen, cyano, amino, carbamoyl, hydroxy, oxo, C₁—C-alkyl-SO₂—, a group 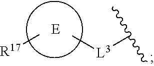
R¹⁵ is selected from hydrogen, halogen, hydroxy, oxo, and C₁-C₆-alkyl;
R¹⁶ is selected from hydrogen and halogen; and
R¹⁷ is selected from hydrogen, C₁-C₆-alkyl, and halo-C₁-C₆-alkyl.

4. (canceled)

5. The compound of formula (I) according to claim 3, or a pharmaceutically acceptable salt thereof, wherein:

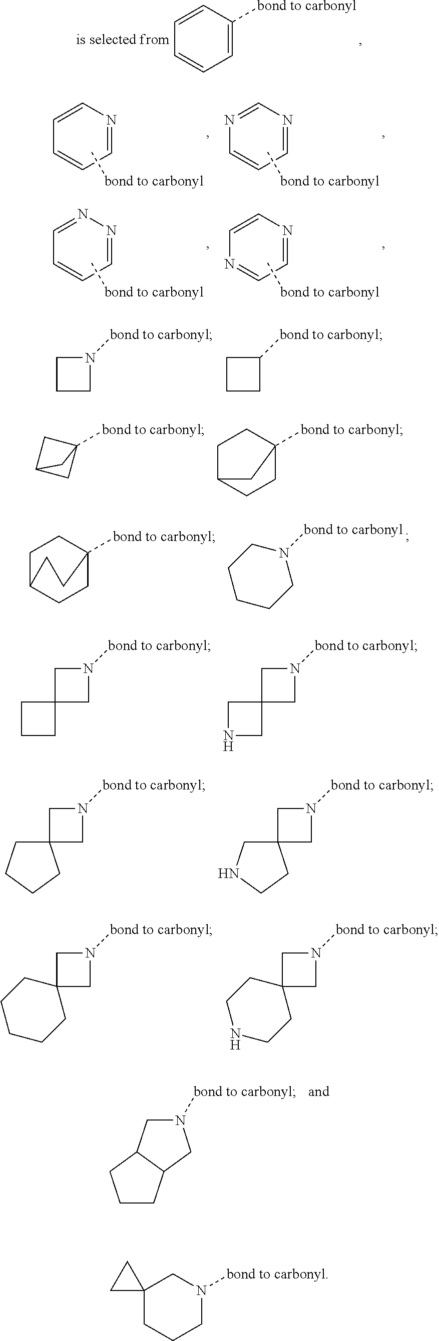

6. The compound of formula (I) according to claim 3, or a pharmaceutically acceptable salt thereof, wherein:
A is selected from phenyl, pyridyl, azetidinyl, 2-azaspiro[3.3]heptan-2-yl, 2,6-diazaspiro[3.3]heptanyl, and 2-azaspiro[3.5]nonan-2-yl;
C is selected from C—C₁₄-aryl, C₃-C₁₀-cycloalkyl, and 5- to 14-membered heteroaryl;
D is selected from C₃-C₁₀-cycloalkyl and 3- to 14-membered heterocyclyl;
L¹ is selected from a covalent bond, —CR¹²R¹³—, —CH₂O—, —O—, —SO₂NH—, and —SO₂—;
L² is selected from a covalent bond and —CH₂—;
R¹ is a group 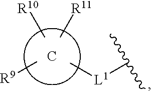
R² is selected from hydrogen and C₁-C₆-alkyl;
R³, R⁴, R¹², and R¹³ are all hydrogen;
R⁹ is selected from halogen, C₁-C₆-alkyl, halo-C₁-C₆-alkyl, halo-C₁-C₆-alkoxy, SF₅, C₁-C₆-alkyl-SO₂—, a group 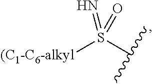
a group 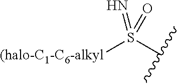
and a group; 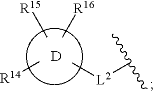
R¹⁰ is selected from hydrogen, halogen, halo-C₁-C₆-alkyl, and C₁-C₆-alkoxy;
R¹¹ is selected from hydrogen and halogen;
R¹⁴ is selected from hydrogen and halo-C₁-C₆-alkyl;
R¹⁵ is selected from hydrogen and hydroxy; and
R¹⁶ is hydrogen.

7. The compound of formula (I) according to claim 6, or a pharmaceutically acceptable salt thereof, wherein:
A is selected from phenyl, pyridyl, azetidinyl, 2-azaspiro[3.3]heptan-2-yl, 2,6-diazaspiro[3.3]heptanyl, and 2-azaspiro[3.5]nonan-2-yl;
C is selected from phenyl, cyclopropyl, pyridyl, 1,2,4-oxadiazolyl, pyrazinyl, and pyrimidinyl;
D is selected from cyclopropyl, azetidinyl, and pyrrolidinyl;
L¹ is selected from a covalent bond, —CR¹²R¹³—, —CH₂O—, —O—, —SO₂NH—, and —SO₂—;
L² is selected from a covalent bond and —CH₂—;
R¹ is a group 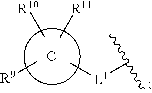
R² is selected from hydrogen and methyl;
R³, R⁴, R¹², and R¹³ are all hydrogen;
R⁹ is selected from fluoro, chloro tert-butyl, CF₃, CF₃O, SF₅, methylsulfonyl, a group 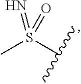 [SMILES: *S(C)(=N)=O]
a group 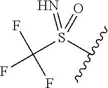 [SMILES: *S(=N)(=O)C(F)(F)F]
and a group 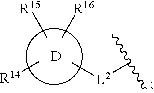
R¹⁰ is selected from hydrogen, fluoro, chloro, CF₃, and methoxy;
R¹¹ is selected from hydrogen and fluoro;
R¹⁴ is selected from hydrogen and CF₃;
R¹⁵ is selected from hydrogen and hydroxy; and
R¹⁶ is hydrogen.

8. The compound of formula (I) according to claim 7, or a pharmaceutically acceptable salt thereof, wherein:
B is a heteroaryl selected from B-1 to B-10: 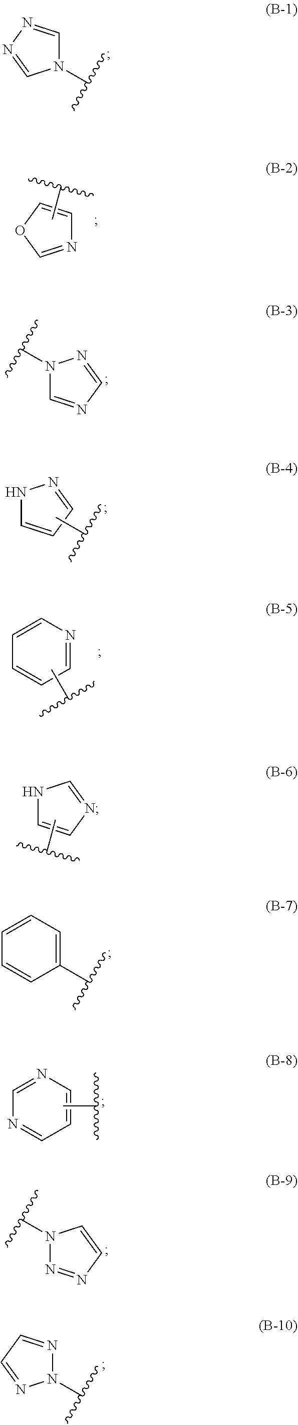
wherein the wavy line indicates the point of attachment to the remainder of formula (I);
R⁵ is selected from hydrogen, halogen, cyano, C₁-C₆-alkyl, C₁-C₆-alkoxy, halo-C₁-C₆-alkyl, C₃-C₁₀-cycloalkyl, and 3- to 14-membered heterocyclyl;
wherein said C₃-C₁₀-cycloalkyl is optionally substituted with one substituent selected from hydroxy and C₁-C₆-alkyl;
R⁶ is selected from hydrogen and halogen; and
R⁷ is absent or hydrogen.

9. The compound of formula (I) according to claim 8, or a pharmaceutically acceptable salt thereof, wherein:
B is 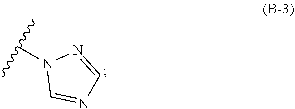 [SMILES: *n1cncn1]
wherein the wavy line indicates the point of attachment to the remainder of formula (I);
R⁵ is selected from C₁-C₆-alkyl, halo-C₁-C₆-alkyl, and C₃-C₁₀-cycloalkyl, wherein said C₃-C₁₀-cycloalkyl is optionally substituted with one hydroxy substituent;
R⁶ is hydrogen; and
R⁷ is absent.

10. The compound of formula (I) according to claim 9, or a pharmaceutically acceptable salt thereof, wherein:
B is 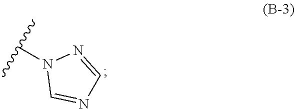 [SMILES: *n1cncn1]
wherein the wavy line indicates the point of attachment to the remainder of formula (I);
R⁵ is selected from ethyl, CF₃, and cyclopropyl, wherein said cyclopropyl is optionally substituted with one hydroxy substituent;
R⁶ is hydrogen; and
R⁷ is absent.

11. The compound of formula (I) according to claim 10, or a pharmaceutically acceptable salt thereof, wherein:
X is CR⁸ or N; and
R⁸ is hydrogen or hydroxy.

12. The compound of formula (I) according to claim 11, or a pharmaceutically acceptable salt thereof, wherein:
X is CR⁸; and
R⁸ is hydrogen.

13. The compound of formula (I) according to claim 1, or a pharmaceutically acceptable salt thereof, wherein:
X is CR⁸ or N;
A is selected from 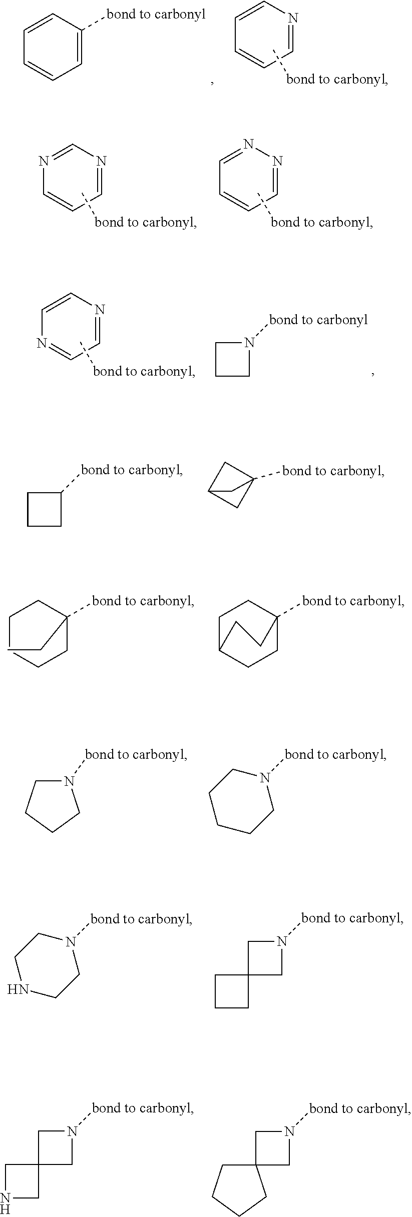 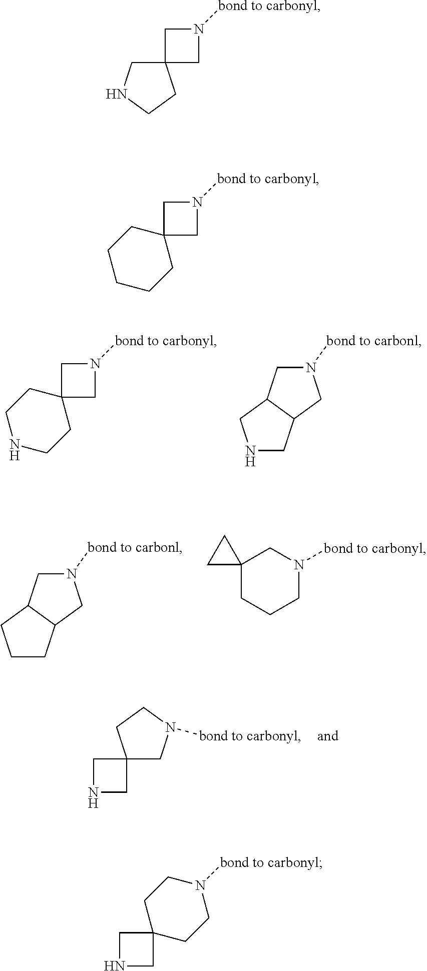
B is a heteroaryl selected from B-1 to B-10: 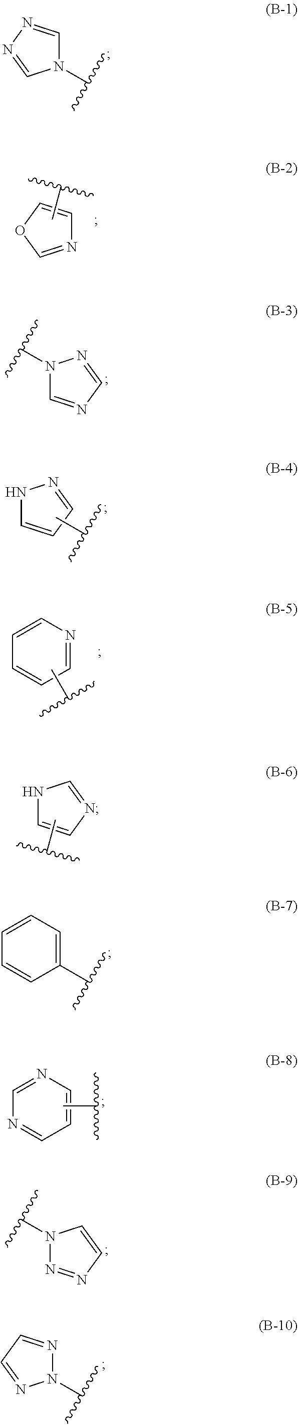
wherein the wavy line indicates the point of attachment to the remainder of formula (I);
C is selected from C₆-C₁₄-aryl, C₃-C₁₀-cycloalkyl, 5- to 6-membered heteroaryl, and 3- to 14-membered heterocyclyl;
D is selected from C₃-C₁₀-cycloalkyl, 3- to 14-membered heterocyclyl, C₆-C₁₄-aryl and 5- to 14-membered heteroaryl;
E is selected from C₃-C₁₀-cycloalkyl, and 3- to 14-membered heterocyclyl;
L¹ is selected from a covalent bond, —CR¹²R¹³—, —CH₂O—, —CH₂NH—, —CH₂OCH₂—, —O—, —NH—, [symbol] , —SO₂NH—, —NHSO₂—, —SO₂NHCH₂—, —CH₂NHSO₂—, —SO₂—, —CH₂SO₂—, —(CH₂)₂SO₂—, carbonyl, and —C(O)NH—;
L² is selected from a covalent bond, —CH₂—, —CH₂NH—, —NHCH₂—, —NH—, —N(C₁-C₆-alkyl)- and —SO₂—;
L³ is selected from a covalent bond and —CH₂—;
R¹ is selected from a group 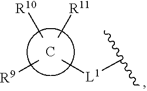
halo-C₁-C₆-alkoxy, C₁-C₆-alkyl-SO₂NH—, C₃-C₁₀-cycloalkyl-C₁-C₆-alkyl-S(O)₂—, C₁—C-alkyl-SO₂—, halo-C₁-C₆-alkyl-S(O)₂—, (C₁-C₆-alkyl)₂N—SO₂—, and halo-C₁-C₆-alkyl-C(O)—;
R² is selected from hydrogen, halogen, C₁-C₆-alkyl, halo-C₁-C₆-alkyl, and 3- to 14-membered heterocyclyl;
R³ is selected from hydrogen and halogen;
R⁴ is hydrogen;
R⁵ is selected from hydrogen, halogen, cyano, C₁-C₆-alkyl, C₁-C₆-alkoxy, halo-C₁-C₆-alkyl, C₃-C₁₀-cycloalkyl, and 3- to 14-membered heterocyclyl;
wherein said C₃-C₁₀-cycloalkyl is optionally substituted with one substituent selected from hydroxy and C₁-C₆-alkyl;
R⁶ is selected from hydrogen and halogen;
R⁷ is absent or hydrogen;
R⁸ is hydrogen or hydroxy;
R⁹ is selected from hydrogen, C₁-C₆-alkyl, C₁-C₆-alkoxy, halo-C₁-C₆-alkyl, halo-C₁-C₆-alkoxy, halogen, cyano, SF₅, C₁—C-alkyl-SO₂—, halo-C₁-C₆-alkyl-SO₂—, (C₁-C₆-alkyl)₂-PO—, amino, carboxy, carboxy-C₁-C₆-alkyl, C₁-C₆-alkoxycarbonyl, C₁-C₆-alkoxycarbonyl-C₁-C₆-alkyl-, NH₂SO₂—, carbamoyl, C₁-C₆-alkyl-C(O)NH—, halo-C₁-C₆-alkyl-NHC(O)—, oxo, a group 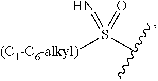
a group 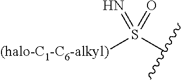
and a group 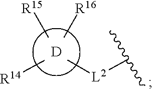
R¹⁰ is selected from hydrogen, halogen, cyano, C₁-C₆-alkyl, C₁-C₆-alkoxy, halo-C₁-C₆-alkyl, and oxo;
R¹¹ is selected from hydrogen and halogen;
R¹² is selected from hydrogen, carbamoyl, C₁-C₆-alkyl-NHC(O)—, and halo-C₆-C₁₄-aryl and R¹³ is hydrogen; or
R¹² and R¹³, taken together with the carbon atom to which they are attached, form a C₃-C₁₀-cycloalkyl;
R¹⁴ is selected from hydrogen, C₁-C₆-alkyl, halo-C₁-C₆-alkyl, C₁-C₆-alkoxy, halo-C₁-C₆-alkoxy, halogen, cyano, amino, carbamoyl, hydroxy, oxo, C₁—C-alkyl-SO₂—, and a group 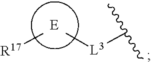
R¹⁵ is selected from hydrogen, halogen, hydroxy, oxo, and C₁-C₆-alkyl;
R¹⁶ is selected from hydrogen and halogen; and
R¹⁷ is selected from hydrogen, C₁-C₆-alkyl, and halo-C₁-C₆-alkyl.

14. The compound of formula (I) according to claim 13, or a pharmaceutically acceptable salt thereof, wherein:
X is CR⁸;
A is selected from phenyl, pyridyl, azetidinyl, 2-azaspiro[3.3]heptan-2-yl, 2,6-diazaspiro[3.3]heptanyl, and 2-azaspiro[3.5]nonan-2-yl;
B is 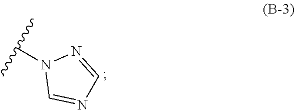 [SMILES: *n1cncn1]
wherein the wavy line indicates the point of attachment to the remainder of formula (I);
C is selected from C₆-C₁₄-aryl, C₃-C₁₀-cycloalkyl, and 5- to 14-membered heteroaryl;
D is selected from C₃-C₁₀-cycloalkyl and 3- to 14-membered heterocyclyl;
L¹ is selected from a covalent bond, —CR¹²R¹³—, —CH₂O—, —O—, —SO₂NH—, and —SO₂—;
L² is selected from a covalent bond and —CH₂—;
R¹ is a group 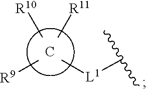
R² is selected from hydrogen and C₁-C₆-alkyl;
R³, R⁴, R⁶, R⁸, R¹², and R¹³ are all hydrogen;
R⁵ is selected from C₁-C₆-alkyl, halo-C₁-C₆-alkyl, and C₃-C₁₀-cycloalkyl, wherein said C₃-C₁₀-cycloalkyl is optionally substituted with one hydroxy substituent;
R⁷ is absent;
R⁹ is selected from halogen, C₁-C₆-alkyl, halo-C₁-C₆-alkyl, halo-C₁-C₆-alkoxy, SF₅, C₁-C₆-alkyl-SO₂—, a group 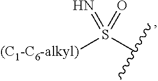
a group 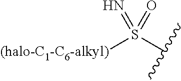
and a group 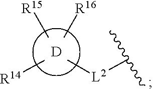
R¹⁰ is selected from hydrogen, halogen, halo-C₁-C₆-alkyl, and C₁-C₆-alkoxy;
R¹¹ is selected from hydrogen and halogen;
R¹⁴ is selected from hydrogen and halo-C₁-C₆-alkyl;
R¹⁵ is selected from hydrogen and hydroxy; and
R¹⁶ is hydrogen.

15. The compound of formula (I) according to claim 14, or a pharmaceutically acceptable salt thereof, wherein:
X is CR⁸;
A is selected from phenyl, pyridyl, azetidinyl, 2-azaspiro[3.3]heptan-2-yl, 2,6-diazaspiro[3.3]heptanyl, and 2-azaspiro[3.5]nonan-2-yl;
B is 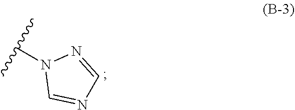 [SMILES: *n1cncn1]
wherein the wavy line indicates the point of attachment to the remainder of formula (I);
C is selected from phenyl, cyclopropyl, pyridyl, 1,2,4-oxadiazolyl, pyrazinyl, and pyrimidinyl;
D is selected from cyclopropyl, azetidinyl, and pyrrolidinyl;
L¹ is selected from a covalent bond, —CR¹²R¹³—, —CH₂O—, —O—, —SO₂NH—, and —SO₂—;
L² is selected from a covalent bond and —CH₂—;
R¹ is a group 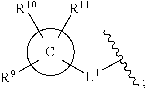
R² is selected from hydrogen and methyl;
R³, R⁴, R⁶, R⁸, R¹², and R¹³ are all hydrogen;
R⁵ is selected from ethyl, CF₃, and cyclpropyl, wherein said cyclopropyl is optionally substituted with one hydroxy substituent;
R⁷ is absent;
R⁹ is selected from fluoro, chloro, tert-butyl, CF₃, CF₃O, SF₅, methylsulfonyl, a group 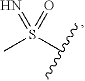 [SMILES: *S(C)(=N)=O]
a group 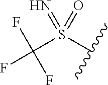 [SMILES: *S(=N)(=O)C(F)(F)F]
and a group 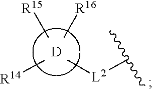
R¹⁰ is selected from hydrogen, fluoro, chloro, CF₃, and methoxy;
R¹¹ is selected from hydrogen and fluoro;
R¹⁴ is selected from hydrogen and CF₃;
R¹⁵ is selected from hydrogen and hydroxy; and
R¹⁶ is hydrogen.

16. The compound of formula (I) according to claim 1, or a pharmaceutically acceptable salt thereof, wherein the compound is selected from:
(6-(3-cyclopropyl-1H-1,2,4-triazol-1-yl)-2-azaspiro[3.3]heptan-2-yl)(3-(4-(1-(trifluoromethyl)cyclopropyl)phenyl)azetidin-1-yl)methanone;
(6-(3-cyclopropyl-1H-1,2,4-triazol-1-yl)-2-azaspiro[3.3]heptan-2-yl)(3-(3-((1-(trifluoromethyl)cyclopropyl)methyl)-1,2,4-oxadiazol-5-yl)azetidin-1-yl)methanone;
(4-(5-(tert-butyl)-1,2,4-oxadiazol-3-yl)phenyl)(6-(3-cyclopropyl-1H-1,2,4-triazol-1-yl)-2-azaspiro[3.3]heptan-2-yl)methanone;
(4-(5-(tert-butyl)-1,2,4-oxadiazol-3-yl)phenyl)(6-(3-(trifluoromethyl)-1H-1,2,4-triazol-1-yl)-2-azaspiro [3.3]heptan-2-yl)methanone;
[4-(5-tert-butyl-1,2,4-oxadiazol-3-yl)phenyl]−[6-[3-(trifluoromethyl)pyrazol-1-yl]-2-azaspiro[3.3]heptan-2-yl]methanone;
[4-(5-tert-butyl-1,2,4-oxadiazol-3-yl)phenyl]−[6-(4-chloropyrazol-1-yl)-2-azaspiro[3.3]heptan-2-yl]methanone;
[4-(5-tert-butyl-1,2,4-oxadiazol-3-yl)phenyl]−[6-(4-cyclopropylpyrazol-1-yl)-2-azaspiro[3.3]heptan-2-yl]methanone;
1-[2-[4-(5-tert-butyl-1,2,4-oxadiazol-3-yl)benzoyl]-2-azaspiro[3.3]heptan-6-yl]pyrazole-3-carbonitrile;
[4-(5-tert-butyl-1,2,4-oxadiazol-3-yl)phenyl]−[6-[3-(1-methylcyclopropyl)pyrazol-1-yl]-2-azaspiro[3.3]heptan-2-yl]methanone;
[4-(5-tert-butyl-1,2,4-oxadiazol-3-yl)phenyl]−[6-[5-(1-methylcyclopropyl)pyrazol-1-yl]-2-azaspiro[3.3]heptan-2-yl]methanone;
[4-(5-tert-butyl-1,2,4-oxadiazol-3-yl)phenyl]−[6-(4-methoxypyrazol-1-yl)-2-azaspiro[3.3]heptan-2-yl]methanone;
[4-(5-tert-butyl-1,2,4-oxadiazol-3-yl)phenyl]−[6-(4,5,6,7-tetrahydroindazol-2-yl)-2-azaspiro[3.3]heptan-2-yl]methanone;
[4-(5-tert-butyl-1,2,4-oxadiazol-3-yl)phenyl]−[6-(3-methoxypyrazol-1-yl)-2-azaspiro[3.3]heptan-2-yl]methanone;
[4-(5-tert-butyl-1,2,4-oxadiazol-3-yl)phenyl]−[6-(6,7-dihydro-4H-pyrano[4,3-c]pyrazol-2-yl)-2-azaspiro[3.3]heptan-2-yl]methanone;
[4-(5-tert-butyl-1,2,4-oxadiazol-3-yl)phenyl]−[6-(4-fluoropyrazol-1-yl)-2-azaspiro[3.3]heptan-2-yl]methanone;
[4-(5-tert-butyl-1,2,4-oxadiazol-3-yl)phenyl]−[6-[5-(trifluoromethyl)pyrazol-1-yl]-2-azaspiro[3.3]heptan-2-yl]methanone;
[4-(5-tert-butyl-1,2,4-oxadiazol-3-yl)phenyl]−[6-(5-cyclopropyl-1,2,4-triazol-1-yl)-2-azaspiro[3.3]heptan-2-yl]methanone;
[4-(5-tert-butyl-1,2,4-oxadiazol-3-yl)phenyl]−[6-(6,7-dihydro-4H-pyrano[4,3-c]pyrazol-1-yl)-2-azaspiro[3.3]heptan-2-yl]methanone;
[4-(5-tert-butyl-1,2,4-oxadiazol-3-yl)phenyl]−[6-(5-methoxypyrazol-1-yl)-2-azaspiro[3.3]heptan-2-yl]methanone;
1-[2-[4-(5-tert-butyl-1,2,4-oxadiazol-3-yl)benzoyl]-2-azaspiro[3.3]heptan-6-yl]-1,2,4-triazole-3-carbonitrile;
1-[2-[4-(5-tert-butyl-1,2,4-oxadiazol-3-yl)benzoyl]-2-azaspiro[3.3]heptan-6-yl]pyrazole-4-carbonitrile;
[4-(5-tert-butyl-1,2,4-oxadiazol-3-yl)phenyl]−[6-(4,5,6,7-tetrahydroindazol-1-yl)-2-azaspiro[3.3]heptan-2-yl]methanone;
[4-(5-tert-butyl-1,2,4-oxadiazol-3-yl)phenyl]−[6-(6-methyl-3-pyridyl)-2-azaspiro[3.3]heptan-2-yl]methanone;
[6-(3-cyclopropyl-1,2,4-triazol-1-yl)-2-azaspiro[3.3]heptan-2-yl]−[5-methyl-6-[[1-(trifluoromethyl)cyclopropyl]methoxy]-3-pyridyl]methanone;
[6-(4-cyclopropylimidazol-1-yl)-2-azaspiro[3.3]heptan-2-yl]−[5-methyl-6-[[1-(trifluoromethyl)cyclopropyl]methoxy]-3-pyridyl]methanone;
[6-(3-cyclopropyl-1,2,4-triazol-1-yl)-2-azaspiro[3.3]heptan-2-yl]−[5-fluoro-6-[[1-(trifluoromethyl)cyclopropyl]methoxy]-3-pyridyl]methanone;
[6-(3-cyclopropyl-1,2,4-triazol-1-yl)-2-azaspiro[3.3]heptan-2-yl]−[6-methyl-5-[[1-(trifluoromethyl)cyclopropyl]methoxy]pyrazin-2-yl]methanone;
[6-(4-cyclopropylimidazol-1-yl)-2-azaspiro[3.3]heptan-2-yl]−[5-fluoro-6-[[1-(trifluoromethyl)cyclopropyl]methoxy]-3-pyridyl]methanone;
[6-(3-cyclopropyl-1,2,4-triazol-1-yl)-2-azaspiro[3.3]heptan-2-yl]−[5-fluoro-6-[(1-methylcyclopropyl)methoxy]-3-pyridyl]methanone;
[[6-(4-cyclopropylimidazol-1-yl)-2-azaspiro[3.3]heptan-2-yl]−[5-fluoro-6-[(1-methylcyclopropyl)methoxy]-3-pyridyl]methanone;
[6-(3-cyclopropyl-1,2,4-triazol-1-yl)-2-azaspiro[3.3]heptan-2-yl]−[2-[[1-(trifluoromethyl)cyclopropyl]methoxy]pyrimidin-5-yl]methanone;
[6-(3-cyclopropyl-1,2,4-triazol-1-yl)-2-azaspiro[3.3]heptan-2-yl]−[5-methyl-6-[[1-(trifluoromethyl)cyclopropyl]methoxy]pyridazin-3-yl]methanone;
[6-(3-cyclopropyl-1,2,4-triazol-1-yl)-2-azaspiro[3.3]heptan-2-yl]−[3-[4-(trifluoromethoxy)phenyl]azetidin-1-yl]methanone;
[6-(3-cyclopropyl-1,2,4-triazol-1-yl)-2-azaspiro[3.3]heptan-2-yl]−[3-[4-(1-morpholinocyclopropyl)phenyl]azetidin-1-yl]methanone;
6-(3-cyclopropyl-1,2,4-triazol-1-yl)-2-azaspiro[3.3]heptan-2-yl]−[3-[4-(1,1-difluoroethyl)phenyl]azetidin-1-yl]methanone;
[6-(3-cyclopropyl-1,2,4-triazol-1-yl)-2-azaspiro[3.3]heptan-2-yl]−[3-[3-fluoro-4-(trifluoromethoxy)phenyl]azetidin-1-yl]methanone;
[6-(3-cyclopropyl-1,2,4-triazol-1-yl)-2-azaspiro[3.3]heptan-2-yl]−[3-[4-(2,2,2-trifluoroethyl)phenyl]azetidin-1-yl]methanone;
[3-(4-cyclopropyl-2-fluoro-phenyl)azetidin-1-yl]−[6-(3-cyclopropyl-1,2,4-triazol-1-yl)-2-azaspiro[3.3]heptan-2-yl]methanone;
5-[1-[6-(3-cyclopropyl-1,2,4-triazol-1-yl)-2-azaspiro[3.3]heptane-2-carbonyl]azetidin-3-yl]-2-(trifluoromethoxy)benzonitrile;
[6-(3-cyclopropyl-1,2,4-triazol-1-yl)-2-azaspiro[3.3]heptan-2-yl]−[6-[[2-methoxy-4-(trifluoromethyl)phenyl]methyl]-2-azaspiro[3.3]heptan-2-yl]methanone;
[6-(3-cyclopropyl-1,2,4-triazol-1-yl)-2-azaspiro[3.3]heptan-2-yl]−[6-[(2,4-difluorophenyl)methyl]-2-azaspiro[3.3]heptan-2-yl]methanone;
6-(3-cyclopropyl-1,2,4-triazol-1-yl)-2-azaspiro[3.3]heptan-2-yl]−[6-[2-fluoro-4-(trifluoromethoxy)phenoxy]-2-azaspiro[3.3]heptan-2-yl]methanone;
[6-(3-cyclopropyl-1,2,4-triazol-1-yl)-2-azaspiro[3.3]heptan-2-yl]−[6-[4-(trifluoromethyl)phenoxy]-2-azaspiro[3.3]heptan-2-yl]methanone;
[3-(4-tert-butylphenyl)azetidin-1-yl]−[6-(3-cyclopropyl-1,2,4-triazol-1-yl)-2-azaspiro[3.3]heptan-2-yl]methanone;
[6-(2-chloro-4-fluoro-phenoxy)-2-azaspiro[3.3]heptan-2-yl]−[6-(3-cyclopropyl-1,2,4-triazol-1-yl)-2-azaspiro[3.3]heptan-2-yl]methanone;
(6-(3-cyclopropyl-1H-1,2,4-triazol-1-yl)-2-azaspiro[3.3]heptan-2-yl)(6-(3-fluoro-5-(trifluoromethyl)phenoxy)-2-azaspiro[3.3]heptan-2-yl)methanone;
2-[[2-[6-(3-cyclopropyl-1,2,4-triazol-1-yl)-2-azaspiro[3.3]heptane-2-carbonyl]-2-azaspiro[3.3]heptan-6-yl]oxy]-5-(trifluoromethoxy)benzonitrile;
[6-(4-cyclopropylimidazol-1-yl)-2-azaspiro[3.3]heptan-2-yl]−[3-[4-[1-(trifluoromethyl)cyclopropyl]phenyl]azetidin-1-yl]methanone;
[6-(3-cyclopropyl-1,2,4-triazol-1-yl)-2-azaspiro[3.3]heptan-2-yl]−[3-[[2-fluoro-4-(pentafluoro-X⁶-sulfanyl)phenyl]methoxy]azetidin-1-yl]methanone;
(6-(3-cyclopropyl-1H-1,2,4-triazol-1-yl)-2-azaspiro[3.3]heptan-2-yl)(6-(3,4-difluorobenzyl)-2-azaspiro[3.3]heptan-2-yl)methanone;
(6-(3-cyclopropyl-1H-1,2,4-triazol-1-yl)-2-azaspiro[3.3]heptan-2-yl)(6-((6-(trifluoromethyl)pyrazin-2-yl)oxy)-2-azaspiro[3.3]heptan-2-yl)methanone;
[6-(3-cyclopropyl-1,2,4-triazol-1-yl)-2-azaspiro[3.3]heptan-2-yl]−[6-[[5-(trifluoromethyl)-2-pyridyl]oxy]-2-azaspiro[3.3]heptan-2-yl]methanone;
[3-[3-chloro-4-(trifluoromethoxy)phenyl]azetidin-1-yl]−[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[6-[4-(trifluoromethoxy)phenoxy]-2-azaspiro[3.3]heptan-2-yl]methanone;
[6-(3-cyclopropyl-1,2,4-triazol-1-yl)-2-azaspiro[3.3]heptan-2-yl]−[6-[2-(trifluoromethyl)pyrimidin-4-yl]oxy-2-azaspiro[3.3]heptan-2-yl]methanone;
(6-(3-cyclopropyl-1H-1,2,4-triazol-1-yl)-2-azaspiro[3.3]heptan-2-yl)(6-((6-(difluoromethoxy)pyridin-3-yl)oxy)-2-azaspiro[3.3]heptan-2-yl)methanone;
[6-(3-cyclopropyl-1,2,4-triazol-1-yl)-2-azaspiro[3.3]heptan-2-yl]−[6-[6-(trifluoromethyl)pyrimidin-4-yl]oxy-2-azaspiro[3.3]heptan-2-yl]methanone;
[6-(3-cyclopropyl-1,2,4-triazol-1-yl)-2-azaspiro[3.3]heptan-2-yl]−[6-[4-(trifluoromethyl)pyrimidin-2-yl]oxy-2-azaspiro[3.3]heptan-2-yl]methanone;
(6-(3-cyclopropyl-1H-1,2,4-triazol-1-yl)-2-azaspiro[3.3]heptan-2-yl)(6-(2,4-difluorophenoxy)-2-azaspiro[3.3]heptan-2-yl)methanone;
(6-(3-cyclopropyl-1H-1,2,4-triazol-1-yl)-2-azaspiro[3.3]heptan-2-yl)(6-((6-methoxypyridin-3-yl)oxy)-2-azaspiro[3.3]heptan-2-yl)methanone;
(6-(3-cyclopropyl-1H-1,2,4-triazol-1-yl)-2-azaspiro[3.3]heptan-2-yl)(6-((2-(trifluoromethyl)pyrimidin-5-yl)oxy)-2-azaspiro[3.3]heptan-2-yl)methanone;
[6-(3-cyclopropyl-1,2,4-triazol-1-yl)-2-azaspiro[3.3]heptan-2-yl]−[6-[6-(trifluoromethyl)pyridazin-3-yl]oxy-2-azaspiro[3.3]heptan-2-yl]methanone;
(6-(3-cyclopropyl-1H-1,2,4-triazol-1-yl)-2-azaspiro[3.3]heptan-2-yl)(6-((5-fluoropyridin-3-yl)oxy)-2-azaspiro[3.3]heptan-2-yl)methanone;
(4-(5-(tert-butyl)-1,2,4-oxadiazol-3-yl)phenyl)(6-(4-cyclopropyl-1H-imidazol-1-yl)-2-azaspiro[3.3]heptan-2-yl)methanone;
[6-(3-cyclopropyl-1,2,4-triazol-1-yl)-2-azaspiro[3.3]heptan-2-yl]−[6-(2,2,2-trifluoroethoxy)-2-azaspiro[3.3]heptan-2-yl]methanone;
4-[[2-[6-(3-cyclopropyl-1,2,4-triazol-1-yl)-2-azaspiro[3.3]heptane-2-carbonyl]-2-azaspiro[3.3]heptan-6-yl]oxy]-1-methyl-pyridin-2-one;
[6-(5-cyclopropyl-1,2,4-triazol-1-yl)-2-azaspiro[3.3]heptan-2-yl]−[3-[4-[1-(trifluoromethyl)cyclopropyl]phenyl]azetidin-1-yl]methanone;
[6-(3-cyclopropyl-1,2,4-triazol-1-yl)-2-azaspiro[3.3]heptan-2-yl]−[6-(3,4-difluorophenoxy)-2-azaspiro[3.3]heptan-2-yl]methanone;
[3-[3-chloro-4-(trifluoromethoxy)phenyl]azetidin-1-yl]−[6-(4-cyclopropylimidazol-1-yl)-2-azaspiro[3.3]heptan-2-yl]methanone;
[6-(3-cyclopropyl-1,2,4-triazol-1-yl)-2-azaspiro[3.3]heptan-2-yl]−[6-[5-(trifluoromethyl)pyrazin-2-yl]oxy-2-azaspiro[3.3]heptan-2-yl]methanone;
(6-(3-cyclopropyl-1H-1,2,4-triazol-1-yl)-2-azaspiro[3.3]heptan-2-yl)(3-((4-(trifluoromethoxy)benzyl)oxy)azetidin-1-yl)methanone;
(6-(3-cyclopropyl-1H-1,2,4-triazol-1-yl)-2-azaspiro[3.3]heptan-2-yl)(3-((4-(trifluoromethyl)benzyl)oxy)azetidin-1-yl)methanone;
(6-(3-cyclopropyl-1H-1,2,4-triazol-1-yl)-2-azaspiro[3.3]heptan-2-yl)(3-((2-fluoro-5-(trifluoromethyl)benzyl)oxy)azetidin-1-yl)methanone;
(6-(3-cyclopropyl-1H-1,2,4-triazol-1-yl)-2-azaspiro[3.3]heptan-2-yl)(3-((3-fluoro-4-(trifluoromethyl)benzyl)oxy)azetidin-1-yl)methanone;
(6-(3-cyclopropyl-1H-1,2,4-triazol-1-yl)-2-azaspiro[3.3]heptan-2-yl)(3-((2-fluoro-4-(trifluoromethoxy)benzyl)oxy)azetidin-1-yl)methanone;
(6-(3-cyclopropyl-1H-1,2,4-triazol-1-yl)-2-azaspiro[3.3]heptan-2-yl)(3-((3-fluoro-5-(trifluoromethyl)benzyl)oxy)azetidin-1-yl)methanone;
(3-((2-chloro-4-fluorobenzyl)oxy)azetidin-1-yl)(6-(3-cyclopropyl-1H-1,2,4-triazol-1-yl)-2-azaspiro[3.3]heptan-2-yl)methanone;
(6-(3-cyclopropyl-1H-1,2,4-triazol-1-yl)-2-azaspiro[3.3]heptan-2-yl)(3-((4-fluoro-2-(trifluoromethyl)benzyl)oxy)azetidin-1-yl)methanone;
(6-(3-cyclopropyl-1H-1,2,4-triazol-1-yl)-2-azaspiro[3.3]heptan-2-yl)(6-(2,5-difluorophenoxy)-2-azaspiro[3.3]heptan-2-yl)methanone;
(6-(3-cyclopropyl-1H-1,2,4-triazol-1-yl)-2-azaspiro[3.3]heptan-2-yl)(6-((6-(trifluoromethyl)pyridin-3-yl)oxy)-2-azaspiro[3.3]heptan-2-yl)methanone;
(6-(3-cyclopropyl-1H-1,2,4-triazol-1-yl)-2-azaspiro[3.3]heptan-2-yl)(6-((5-(trifluoromethyl)pyridin-3-yl)oxy)-2-azaspiro[3.3]heptan-2-yl)methanone;
(6-((5-chloropyridin-3-yl)oxy)-2-azaspiro[3.3]heptan-2-yl)(6-(3-cyclopropyl-1H-1,2,4-triazol-1-yl)-2-azaspiro[3.3]heptan-2-yl)methanone;
(6-((6-chloropyridin-3-yl)oxy)-2-azaspiro[3.3]heptan-2-yl)(6-(3-cyclopropyl-1H-1,2,4-triazol-1-yl)-2-azaspiro[3.3]heptan-2-yl)methanone;
[3-[2-chloro-4-(trifluoromethoxy)phenyl]azetidin-1-yl]−[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[3-[3,5-difluoro-4-(trifluoromethoxy)phenyl]azetidin-1-yl]methanone;
[6-(3-cyclopropyl-1,2,4-triazol-1-yl)-2-azaspiro[3.3]heptan-2-yl]−[3-[2-fluoro-4-(trifluoromethoxy)phenyl]azetidin-1-yl]methanone;
[3-(2-tert-butylthiazol-4-yl)azetidin-1-yl]−[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[4-[5-(2,2-dimethylpropyl)-1,2,4-oxadiazol-3-yl]phenyl]methanone;
[6-(3-cyclopropyl-1,2,4-triazol-1-yl)-2-azaspiro[3.3]heptan-2-yl]−[rac-(3aS,6aR)-5-[2-fluoro-4-(trifluoromethyl)phenoxy]-3,3a,4,5,6,6a-hexahydro-1H-cyclopenta[c]pyrrol-2-yl]methanone;
[4-(5-tert-butyl-1,2,4-oxadiazol-3-yl)phenyl]−[6-(3-cyclobutyl-1,2,4-triazol-1-yl)-2-azaspiro[3.3]heptan-2-yl]methanone;
[6-(4-cyclopropylimidazol-1-yl)-2-azaspiro[3.3]heptan-2-yl]−[rac-(3aR,6aS)-5-(2-chloro-4-fluoro-phenoxy)-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[3-[2-[4-(difluoromethoxy)phenyl]ethynyl]azetidin-1-yl]methanone;
[6-(3-cyclopropyl-1,2,4-triazol-1-yl)-2-azaspiro[3.3]heptan-2-yl]−[rac-(3aR,6aS)-5-(2-chloro-4-fluoro-phenoxy)-3,3a,4,5,6,6a-hexahydro-1H-cyclopenta[c]pyrrol-2-yl]methanone;
(3-(2-chloro-3-cyclopropylphenoxy)azetidin-1-yl)(6-(3-cyclopropyl-1H-1,2,4-triazol-1-yl)-2-azaspiro[3.3]heptan-2-yl)methanone;
(3-(4-chloro-3-cyclopropylphenoxy)azetidin-1-yl)(6-(3-cyclopropyl-1H-1,2,4-triazol-1-yl)-2-azaspiro[3.3]heptan-2-yl)methanone;
(6-(3-cyclopropyl-1H-1,2,4-triazol-1-yl)-2-azaspiro[3.3]heptan-2-yl)(3-(2-fluoro-4-(trifluoromethyl)phenoxy)azetidin-1-yl)methanone;
(3-(2-chloro-4-methylphenoxy)azetidin-1-yl)(6-(3-cyclopropyl-1H-1,2,4-triazol-1-yl)-2-azaspiro[3.3]heptan-2-yl)methanone;
(6-(3-cyclopropyl-1H-1,2,4-triazol-1-yl)-2-azaspiro[3.3]heptan-2-yl)(3-(2,4-dichlorophenoxy)azetidin-1-yl)methanone;
(3-(4-chloro-2-fluorophenoxy)azetidin-1-yl)(6-(3-cyclopropyl-1H-1,2,4-triazol-1-yl)-2-azaspiro[3.3]heptan-2-yl)methanone;
(3-((2-chloro-6-methylpyridin-3-yl)oxy)azetidin-1-yl)(6-(3-cyclopropyl-1H-1,2,4-triazol-1-yl)-2-azaspiro[3.3]heptan-2-yl)methanone;
[6-(4-cyclopropylimidazol-1-yl)-2-azaspiro[3.3]heptan-2-yl]−[3-[[4-(trifluoromethyl)phenyl]methoxy]azetidin-1-yl]methanone;
[6-(4-cyclopropylimidazol-1-yl)-2-azaspiro[3.3]heptan-2-yl]−[3-[[2-fluoro-4-(trifluoromethyl)phenyl]methoxy]azetidin-1-yl]methanone;
[6-(4-cyclopropylimidazol-1-yl)-2-azaspiro[3.3]heptan-2-yl]−[3-[[3-fluoro-4-(trifluoromethyl)phenyl]methoxy]azetidin-1-yl]methanone;
[6-(4-cyclopropylimidazol-1-yl)-2-azaspiro[3.3]heptan-2-yl]−[3-[[4-(pentafluoro-λ⁶-sulfanyl)phenyl]methoxy]azetidin-1-yl]methanone;
[6-(4-cyclopropylimidazol-1-yl)-2-azaspiro[3.3]heptan-2-yl]−[3-[[2-fluoro-4-(pentafluoro-)⁶-sulfanyl)phenyl]methoxy]azetidin-1-yl]methanone;
[6-(4-cyclopropylimidazol-1-yl)-2-azaspiro[3.3]heptan-2-yl]−[3-[[4-methyl-3-(trifluoromethyl)phenyl]methoxy]azetidin-1-yl]methanone;
[6-(3-cyclopropyl-1,2,4-triazol-1-yl)-2-azaspiro[3.3]heptan-2-yl]−[3-[2-[2-(difluoromethyl)phenyl]ethynyl]azetidin-1-yl]methanone;
[3-[(4-chloro-2-fluoro-phenyl)methoxy]azetidin-1-yl]−[6-(4-cyclopropylimidazol-1-yl)-2-azaspiro[3.3]heptan-2-yl]methanone;
[3-[(2-chloro-4-fluoro-phenyl)methoxy]azetidin-1-yl]−[6-(4-cyclopropylimidazol-1-yl)-2-azaspiro[3.3]heptan-2-yl]methanone;
[6-(4-cyclopropylimidazol-1-yl)-2-azaspiro[3.3]heptan-2-yl]−[3-[(2,4-difluorophenyl)methoxy]azetidin-1-yl]methanone;
4-[[2-[6-(3-cyclopropyl-1,2,4-triazol-1-yl)-2-azaspiro[3.3]heptane-2-carbonyl]-2-azaspiro[3.3]heptan-6-yl]oxy]-3-fluoro-benzonitrile;
[6-(3-cyclopropyl-1,2,4-triazol-1-yl)-2-azaspiro[3.3]heptan-2-yl]−[rac-(3aS,6aR)-5-[4-(difluoromethoxy)-2-fluoro-phenoxy]-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-[4-(trifluoromethyl)phenoxy]-3,3a,4,5,6,6a-hexahydro-1H-cyclopenta[c]pyrrol-2-yl]methanone;
2-[[rac-(3aS,6aR)-2-[6-(3-cyclopropyl-1,2,4-triazol-1-yl)-2-azaspiro[3.3]heptane-2-carbonyl]-3,3a,4,5,6,6a-hexahydro-1H-cyclopenta[c]pyrrol-5-yl]oxy]-5-(trifluoromethyl)benzonitrile;
[6-(3-cyclopropyl-1,2,4-triazol-1-yl)-2-azaspiro[3.3]heptan-2-yl]−[rac-(3aS,6aR)-5-[3-chloro-4-(trifluoromethyl)phenoxy]-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-[2-methoxy-4-(trifluoromethyl)phenoxy]-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-[3-fluoro-4-(trifluoromethyl)phenoxy]-3,3a,4,5,6,6a-hexahydro-1H-cyclopenta[c]pyrrol-2-yl]methanone;
5-[[rac-(3aS,6aR)-2-[6-(3-cyclopropyl-1,2,4-triazol-1-yl)-2-azaspiro[3.3]heptane-2-carbonyl]-3,3a,4,5,6,6a-hexahydro-1H-cyclopenta[c]pyrrol-5-yl]oxy]-2-(trifluoromethyl)benzonitrile;
[6-(3-cyclopropyl-1,2,4-triazol-1-yl)-2-azaspiro[3.3]heptan-2-yl]−[rac-(3aS,6aR)-5-(3,4-difluorophenoxy)-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-[4-fluoro-3-(trifluoromethyl)phenoxy]-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-(2,4-difluorophenoxy)-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-(4-fluoro-2-methoxy-phenoxy)-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-(4-fluoro-2-methylsulfonyl-phenoxy)-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-(2,4,6-trifluorophenoxy)-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-(4-fluoro-3-methyl-phenoxy)-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-(4-fluoro-3-chloro-phenoxy)-3,3a,4,5,6,6a-hexahydro-1H-cyclopenta[c]pyrrol-2-yl]methanone;
2-fluoro-5-[[rac-(3aS,6aR)-2-[6-(3-cyclopropyl-1,2,4-triazol-1-yl)-2-azaspiro[3.3]heptane-2-carbonyl]-3,3a,4,5,6,6a-hexahydro-1H-cyclopenta[c]pyrrol-5-yl]oxy]benzonitrile;
[6-(3-cyclopropyl-1,2,4-triazol-1-yl)-2-azaspiro[3.3]heptan-2-yl]−[rac-(3aS,6aR)-5-(4,5-difluoro-2-methyl-phenoxy)-3,3a,4,5,6,6a-hexahydro-1H-cyclopenta[c]pyrrol-2-yl]methanone;
5-fluoro-2-[[rac-(3aS,6aR)-2-[6-(3-cyclopropyl-1,2,4-triazol-1-yl)-2-azaspiro[3.3]heptane-2-carbonyl]-3,3a,4,5,6,6a-hexahydro-1H-cyclopenta[c]pyrrol-5-yl]oxy]benzonitrile;
[6-(3-cyclopropyl-1,2,4-triazol-1-yl)-2-azaspiro[3.3]heptan-2-yl]−[rac-(3aS,6aR)-5-(4-fluoro-3-methylsulfonyl-phenoxy)-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-(4-fluoro-3-methoxy-phenoxy)-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-[2,4-difluoro-5-(trifluoromethyl)phenoxy]-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-[4-fluoro-3-(trifluoromethoxy)phenoxy]-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-[4-(trifluoromethoxy)phenoxy]-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[rac-(3aS,6aR)-5-[3-fluoro-4-(trifluoromethoxy)phenoxy]-3,3a,4,5,6,6a-hexahydro-1H-cyclopenta[c]pyrrol-2-yl]methanone;
[6-(3-cyclopropyl-1,2,4-triazol-1-yl)-2-azaspiro[3.3]heptan-2-yl]−[3-[[5-(trifluoromethyl)-3-pyridyl]methoxy]azetidin-1-yl]methanone;
[6-(3-cyclopropyl-1,2,4-triazol-1-yl)-2-azaspiro[3.3]heptan-2-yl]−[6-[[1-(trifluoromethyl)cyclopropyl]methoxy]-2-azaspiro[3.3]heptan-2-yl]methanone;
[6-(3-cyclopropyl-1,2,4-triazol-1-yl)-2-azaspiro[3.3]heptan-2-yl]−[2-methyl-3-[[4-(trifluoromethyl)phenyl]methoxy]azetidin-1-yl]methanone;
[6-(4-cyclopropylimidazol-1-yl)-2-azaspiro[3.3]heptan-2-yl]−[3-[2-[2-(difluoromethyl)phenyl]ethynyl]azetidin-1-yl]methanone;
[6-(3-cyclopropyl-1,2,4-triazol-1-yl)-2-azaspiro[3.3]heptan-2-yl]−[3-[5-[1-(trifluoromethyl)cyclopropyl]-1,2,4-oxadiazol-3-yl]azetidin-1-yl]methanone;
[6-(4-cyclopropylimidazol-1-yl)-2-azaspiro[3.3]heptan-2-yl]−[3-[5-[1-(trifluoromethyl)cyclopropyl]-1,2,4-oxadiazol-3-yl]azetidin-1-yl]methanone;
[6-(4-cyclopropylimidazol-1-yl)-2-azaspiro[3.3]heptan-2-yl]−[5-methyl-6-[(1-methylcyclopropyl)methoxy]-3-pyridyl]methanone;
[6-(3-cyclopropyl-1,2,4-triazol-1-yl)-2-azaspiro[3.3]heptan-2-yl]−[5-methyl-6-[(1-methylcyclopropyl)methoxy]-3-pyridyl]methanone;
[6-(4-cyclopropylimidazol-1-yl)-2-azaspiro[3.3]heptan-2-yl]−[3-[4-(2,2,2-trifluoroethoxy)pyrazol-1-yl]azetidin-1-yl]methanone;
[6-(3-cyclopropyl-1,2,4-triazol-1-yl)-2-azaspiro[3.3]heptan-2-yl]−[3-[5-[1-(trifluoromethyl)cyclopropyl]-1,3,4-oxadiazol-2-yl]azetidin-1-yl]methanone;
[4-(5-tert-butyl-1,2,4-oxadiazol-3-yl)phenyl]−[6-(4-methylpyrazol-1-yl)-2-azaspiro[3.3]heptan-2-yl]methanone;
[6-(4-methylpyrazol-1-yl)-2-azaspiro[3.3]heptan-2-yl]−[5-methyl-6-[[1-(trifluoromethyl)cyclopropyl]methoxy]-3-pyridyl]methanone;
[4-(5-tert-butyl-1,2,4-oxadiazol-3-yl)phenyl]−[6-(3-methylpyrazol-1-yl)-2-azaspiro[3.3]heptan-2-yl]methanone;
[6-(3-cyclopropyl-1,2,4-triazol-1-yl)-2-azaspiro[3.3]heptan-2-yl]−[8-[[1-(trifluoromethyl)cyclopropyl]methoxy]-5-azaspiro[2.5]octan-5-yl]methanone;
[6-(3-cyclopropyl-1,2,4-triazol-1-yl)-2-azaspiro[3.3]heptan-2-yl]−[3,3-difluoro-4-[[1-(trifluoromethyl)cyclopropyl]methoxy]-1-piperidyl]methanone;
[6-(3-cyclopropylpyrazol-1-yl)-2-azaspiro[3.3]heptan-2-yl]−[5-methyl-6-[[1-(trifluoromethyl)cyclopropyl]methoxy]-3-pyridyl]methanone;
[6-(3-cyclopropylpyrazol-1-yl)-2-azaspiro[3.3]heptan-2-yl]−[5-methyl-6-(2,2,2-trifluoro-1,1-dimethyl-ethoxy)-3-pyridyl]methanone;
[6-(3-cyclopropyl-1,2,4-triazol-1-yl)-2-azaspiro[3.3]heptan-2-yl]−[5-(trifluoromethyl)-6-[[1-(trifluoromethyl)cyclopropyl]methoxy]-3-pyridyl]methanone;
[6-(3-cyclopropyl-1,2,4-triazol-1-yl)-2-azaspiro[3.3]heptan-2-yl]−[5-(oxetan-3-yl)-6-[[1-(trifluoromethyl)cyclopropyl]methoxy]-3-pyridyl]methanone;
[6-(3-cyclopropyl-1,2,4-triazol-1-yl)-2-azaspiro[3.3]heptan-2-yl]−[4-[[1-(trifluoromethyl)cyclopropyl]methoxymethyl]-1-bicyclo[2.2.2]octanyl]methanone;
[4-(5-tert-butyl-1,2,4-oxadiazol-3-yl)phenyl]−[6-(3-ethyl-1,2,4-triazol-1-yl)-2-azaspiro[3.3]heptan-2-yl]methanone;
[4-(5-tert-butyl-1,2,4-oxadiazol-3-yl)phenyl]−[6-(4-ethylimidazol-1-yl)-2-azaspiro[3.3]heptan-2-yl]methanone;
[4-(5-tert-butyl-1,2,4-oxadiazol-3-yl)phenyl]−[6-[4-(trifluoromethyl)imidazol-1-yl]-2-azaspiro[3.3]heptan-2-yl]methanone;
[4-(5-tert-butyl-1,2,4-oxadiazol-3-yl)phenyl]−[6-(4-chloroimidazol-1-yl)-2-azaspiro[3.3]heptan-2-yl]methanone;
1-[2-[4-(5-tert-butyl-1,2,4-oxadiazol-3-yl)benzoyl]-2-azaspiro[3.3]heptan-6-yl]imidazole-4-carbonitrile;
[6-(3-cyclopropyl-1,2,4-triazol-1-yl)-2-azaspiro[3.3]heptan-2-yl]−[4-[[1-(trifluoromethyl)cyclopropyl]methoxymethyl]norbornan-1-yl]methanone;
[3-(5-tert-butyl-1,2,4-oxadiazol-3-yl)-1-bicyclo[1.1.1]pentanyl]−[6-(4-cyclopropylimidazol-1-yl)-2-azaspiro[3.3]heptan-2-yl]methanone;
[3-(5-tert-butyl-1,2,4-oxadiazol-3-yl)-1-bicyclo[1.1.1]pentanyl]−[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[3-[1-[1-(trifluoromethyl)cyclopropyl]triazol-4-yl]azetidin-1-yl]methanone;
[6-(3-cyclopropyl-1,2,4-triazol-1-yl)-2-azaspiro[3.3]heptan-2-yl]−[3-[4-(2,2,2-trifluoroethoxy)pyrazol-1-yl]azetidin-1-yl]methanone;
[6-(3-cyclopropyl-1,2,4-triazol-1-yl)-2-azaspiro[3.3]heptan-2-yl]−[3-[3-[1-(trifluoromethyl)cyclopropyl]-1,2,4-oxadiazol-5-yl]azetidin-1-yl]methanone;
[6-(3-cyclopropyl-1,2,4-triazol-1-yl)-2-azaspiro[3.3]heptan-2-yl]−[3-[1-[3-(trifluoromethyl)oxetan-3-yl]triazol-4-yl]azetidin-1-yl]methanone;
[4-(5-tert-butyl-1,3,4-oxadiazol-2-yl)phenyl]−[6-(3-chloro-1,2,4-triazol-1-yl)-2-azaspiro[3.3]heptan-2-yl]methanone;
[4-(5-tert-butyl-1,3,4-oxadiazol-2-yl)phenyl]−[6-(3-cyclopropylpyrazol-1-yl)-2-azaspiro[3.3]heptan-2-yl]methanone;
[4-(1-tert-butylpyrazol-4-yl)phenyl]−[6-(3-chloro-1,2,4-triazol-1-yl)-2-azaspiro[3.3]heptan-2-yl]methanone;
[4-(5-tert-butyl-1,2,4-oxadiazol-3-yl)phenyl]−[6-(3-cyclopropylpyrazol-1-yl)-2-azaspiro[3.3]heptan-2-yl]methanone;
[4-(5-tert-butyl-1,2,4-oxadiazol-3-yl)phenyl]−[6-(3-chloro-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[3-[[1-(trifluoromethyl)cyclopropyl]methoxymethyl]cyclobutyl]methanone;
[6-(3-cyclopropylpyrazol-1-yl)-2-azaspiro[3.3]heptan-2-yl]−[5-methyl-6-(2,2,2-trifluoro-1,1-dimethyl-ethoxy)-3-pyridyl]methanone;
[6-(3-chloro-1,2,4-triazol-1-yl)-2-azaspiro[3.3]heptan-2-yl]−[5-methyl-6-(2,2,2-trifluoro-1,1-dimethyl-ethoxy)-3-pyridyl]methanone;
[6-(3-cyclopropyl-1,2,4-triazol-1-yl)-2-azaspiro[3.3]heptan-2-yl]−[3-[[1-(trifluoromethyl)cyclopropyl]methoxy]cyclobutyl]methanone;
(6-(3-(azetidin-1-yl)-1H-1,2,4-triazol-1-yl)-2-azaspiro[3.3]heptan-2-yl)(3-((4-(trifluoromethyl)benzyl)oxy)azetidin-1-yl)methanone;
[6-(3-cyclopropyl-1,2,4-triazol-4-yl)-2-azaspiro[3.3]heptan-2-yl]−[rac-(3aS,6aR)-5-(2-chloro-4-fluoro-phenoxy)-3,3a,4,5,6,6a-hexahydro-1H-cyclopenta[c]pyrrol-2-yl]methanone;
[4-(5-tert-butyl-1,2,4-oxadiazol-3-yl)phenyl]−[6-(2-cyclopropyloxazol-5-yl)-6-hydroxy-2-azaspiro[3.3]heptan-2-yl]methanone;
[4-(5-tert-butyl-1,2,4-oxadiazol-3-yl)phenyl]−[6-(5-cyclopropylpyrazol-1-yl)-2-azaspiro[3.3]heptan-2-yl]methanone;
[3-(1-tert-butylpyrazol-4-yl)azetidin-1-yl]−[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[3-[3-methyl-4-(trifluoromethoxy)phenyl]azetidin-1-yl]methanone;
(6-(3-cyclopropyl-1H-1,2,4-triazol-1-yl)-2-azaspiro[3.3]heptan-2-yl)(3-(4-cyclopropylphenoxy)azetidin-1-yl)methanone;
[6-(3-cyclobutyl-1,2,4-triazol-1-yl)-2-azaspiro[3.3]heptan-2-yl]−[6-[(2,4-difluorophenyl)methyl]-2-azaspiro[3.3]heptan-2-yl]methanone;
(3-((3-chloro-4-cyclopropylpyridin-2-yl)oxy)azetidin-1-yl)(6-(3-cyclopropyl-1H-1,2,4-triazol-1-yl)-2-azaspiro[3.3]heptan-2-yl)methanone;
[6-[(2,4-difluorophenyl)methyl]-2-azaspiro[3.3]heptan-2-yl]−[6-(3-ethyl-1,2,4-triazol-1-yl)-2-azaspiro[3.3]heptan-2-yl]methanone;
(6-(3-cyclopropyl-1H-1,2,4-triazol-1-yl)-2-azaspiro[3.3]heptan-2-yl)(3-(3-cyclopropyl-4-(trifluoromethyl)phenoxy)azetidin-1-yl)methanone;
5-cyclopropyl-2-((1-(6-(3-cyclopropyl-1H-1,2,4-triazol-1-yl)-2-azaspiro[3.3]heptane-2-carbonyl)azetidin-3-yl)oxy)benzonitrile;
[6-(3-cyclopropyl-1,2,4-triazol-1-yl)-2-azaspiro[3.3]heptan-2-yl]−[6-[2-fluoro-4-(trifluoromethyl)benzyl]-2,6-diazaspiro[3.3]heptan-2-yl]methanone;
(6-(3-cyclopropyl-1H-1,2,4-triazol-1-yl)-2-azaspiro[3.3]heptan-2-yl)(3-(4-cyclopropyl-2-fluorophenoxy)azetidin-1-yl)methanone;
2-cyclopropyl-6-((1-(6-(3-cyclopropyl-1H-1,2,4-triazol-1-yl)-2-azaspiro[3.3]heptane-2-carbonyl)azetidin-3-yl)oxy)benzonitrile;
[6-(3-cyclopropyl-1,2,4-triazol-1-yl)-2-azaspiro[3.3]heptan-2-yl]−[6-(3-mesylbenzyl)-2-azaspiro[3.3]heptan-2-yl]methanone;
[6-(3-cyclopropyl-1,2,4-triazol-1-yl)-2-azaspiro[3.3]heptan-2-yl]−[6-[[5-(trifluoromethyl)pyrazin-2-yl]methyl]-2-azaspiro[3.3]heptan-2-yl]methanone;
methyl 3-[3-[1-[6-(3-cyclopropyl-1,2,4-triazol-1-yl)-2-azaspiro[3.3]heptane-2-carbonyl]azetidin-3-yl]oxyphenyl]-2,2-dimethylpropanoate;
N-[2-[6-(3-cyclopropyl-1,2,4-triazol-1-yl)-2-azaspiro[3.3]heptane-2-carbonyl]-2-azaspiro[3.3]heptan-6-yl]-3-(trifluoromethyl)benzenesulfonamide;
[6-(3-cyclopropyl-1,2,4-triazol-1-yl)-2-azaspiro[3.3]-2-yl]−[6-[[4-fluoro-2-(trifluoromethyl)phenyl]methyl]-2,6-diazaspiro[3.3]heptan-2-yl]methanone;
[4-[(R)-(3-cyclopropyl-1,2,4-oxadiazol-5-yl)-(4-fluorophenyl)methyl]-1-piperidyl]−[6-(3-cyclopropyl-1,2,4-triazol-1-yl)-2-azaspiro[3.3]heptan-2-yl]methanone;
(6-(3-cyclopropyl-1H-1,2,4-triazol-1-yl)-2-azaspiro[3.3]heptan-2-yl)(3-(3-cyclopropyl-2-fluorophenoxy)azetidin-1-yl)methanone;
[6-(3-cyclopropyl-1,2,4-triazol-1-yl)-2-azaspiro[3.3]heptan-2-yl]−[6-[(3,5-difluoro-2-pyridyl)methyl]-2-azaspiro[3.3]heptan-2-yl]methanone;
methyl 2-[3-[1-[6-(3-cyclopropyl-1,2,4-triazol-1-yl)-2-azaspiro[3.3]heptane-2-carbonyl]azetidin-3-yl]oxyphenyl]-2-methylpropanoate;
(6-(2-chloro-4-fluorobenzyl)-2,6-diazaspiro[3.3]heptan-2-yl)(6-(3-cyclopropyl-1H-1,2,4-triazol-1-yl)-2-azaspiro[3.3]heptan-2-yl)methanone;
(6-(3-cyclopropyl-1H-1,2,4-triazol-1-yl)-2-azaspiro[3.3]heptan-2-yl)(3-(4-(trifluoromethyl)phenoxy)azetidin-1-yl)methanone;
[6-[(2,4-difluorophenyl)methyl]-2-azaspiro[3.3]heptan-2-yl]−[6-(3-isopropyl-1,2,4-triazol-1-yl)-2-azaspiro[3.3]heptan-2-yl]methanone;
(6-(3-cyclopropyl-1H-1,2,4-triazol-1-yl)-2-azaspiro[3.3]heptan-2-yl)(3-((4-cyclopropyl-3-fluoropyridin-2-yl)oxy)azetidin-1-yl)methanone;
[6-(3-cyclopropyl-1,2,4-triazol-1-yl)-2-azaspiro[3.3]heptan-2-yl]−[6-(4-mesylbenzyl)-2-azaspiro[3.3]heptan-2-yl]methanone;
[6-(3-cyclopropyl-1,2,4-triazol-1-yl)-2-azaspiro[3.3]heptan-2-yl]−[3-[6-[3-hydroxy-3-(trifluoromethyl)azetidin-1-yl]-3-pyridyl]azetidin-1-yl]methanone;
(4-((3-cyclopropyl-1,2,4-oxadiazol-5-yl)(4-fluorophenyl)methyl)piperidin-1-yl)(6-(3-cyclopropyl-1H-1,2,4-triazol-1-yl)-2-azaspiro[3.3]heptan-2-yl)methanone;
(6-(3-cyclopropyl-1H-1,2,4-triazol-1-yl)-2-azaspiro[3.3]heptan-2-yl)(3-((6-cyclopropyl-2-fluoropyridin-3-yl)oxy)azetidin-1-yl)methanone;
[6-(3-cyclopropyl-1,2,4-triazol-1-yl)-2-azaspiro[3.3]heptan-2-yl]−[2-[3-(trifluoromethyl)phenyl]sulfonyl-2,6-diazaspiro[3.3]heptan-6-yl]methanone;
[3-[(5-cyclopropyl-2-pyridyl)oxy]azetidin-1-yl]−[6-(3-cyclopropyl-1,2,4-triazol-1-yl)-2-azaspiro[3.3]heptan-2-yl]methanone;
(6-(3-cyclopropyl-1H-1,2,4-triazol-1-yl)-2-azaspiro[3.3]heptan-2-yl)(6-((4-fluoro-2-(trifluoromethyl)phenyl)sulfonyl)-2,6-diazaspiro[3.3]heptan-2-yl)methanone;
[6-(3-cyclopropyl-1,2,4-triazol-1-yl)-2-azaspiro[3.3]heptan-2-yl]−[3-[6-[3-hydroxy-3-(trifluoromethyl)pyrrolidino]-3-pyridyl]azetidin-1-yl]methanone;
[6-(3-cyclopropyl-1,2,4-triazol-1-yl)-2-azaspiro[3.3]heptan-2-yl]−[2-(3,5-difluorophenyl)sulfonyl-2,6-diazaspiro[3.3]heptan-6-yl]methanone;
[6-(3-cyclopropyl-1,2,4-triazol-1-yl)-2-azaspiro[3.3]heptan-2-yl]−[3-[6-[(3R)-3-hydroxy-3-(trifluoromethyl)pyrrolidin-1-yl]-3-pyridyl]azetidin-1-yl]methanone;
N-[2-[6-(3-cyclopropyl-1,2,4-triazol-1-yl)-2-azaspiro[3.3]heptane-2-carbonyl]-2-azaspiro[3.3]heptan-6-yl]-2,2-dimethyl-propane-1-sulfonamide;
[6-(3-cyclopropyl-1,2,4-triazol-1-yl)-2-azaspiro[3.3]heptan-2-yl]−[3-[6-[(3S)-3-hydroxy-3-(trifluoromethyl)pyrrolidino]-3-pyridyl]azetidin-1-yl]methanone;
[6-(3-cyclopropyl-1,2,4-triazol-1-yl)-2-azaspiro[3.3]heptan-2-yl]−[6-[(5-fluoro-2-pyridyl)methyl]-2-azaspiro[3.3]heptan-2-yl]methanone;
[6-[(2,4-difluorophenyl)methyl]-2-azaspiro[3.3]heptan-2-yl]−[6-(4-methylimidazol-1-yl)-2-azaspiro[3.3]heptan-2-yl]methanone;
(6-(3-cyclopropyl-1H-1,2,4-triazol-1-yl)-2-azaspiro[3.3]heptan-2-yl)(3-(2-methoxy-3-(trifluoromethyl)phenoxy)azetidin-1-yl)methanone;
(6-(3-cyclopropyl-1H-1,2,4-triazol-1-yl)-2-azaspiro[3.3]heptan-2-yl)(3-(3-cyclopropyl-4-fluorophenoxy)azetidin-1-yl)methanone;
[6-(3-cyclopropyl-1,2,4-triazol-1-yl)-2-azaspiro[3.3]heptan-2-yl]−[6-(3,5-difluorobenzyl)-2,6-diazaspiro[3.3]heptan-2-yl]methanone;
(3-(2-chloro-3-(trifluoromethyl)phenoxy)azetidin-1-yl)(6-(3-cyclopropyl-1H-1,2,4-triazol-1-yl)-2-azaspiro[3.3]heptan-2-yl)methanone;
[6-(3-cyclopropyl-1,2,4-triazol-1-yl)-2-azaspiro[3.3]heptan-2-yl]−[2-[2-(trifluoromethyl)phenyl]sulfonyl-2,6-diazaspiro[3.3]heptan-6-yl]methanone;
(2-cyclohexylsulfonyl-2,6-diazaspiro[3.3]heptan-6-yl)-[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[2-[4-(trifluoromethyl)phenyl]sulfonyl-2,6-diazaspiro[3.3]heptan-6-yl]methanone;
[6-(3-cyclopropyl-1,2,4-triazol-1-yl)-2-azaspiro[3.3]heptan-2-yl]−[2-[3-fluoro-5-(trifluoromethyl)phenyl]sulfonyl-2,6-diazaspiro[3.3]heptan-6-yl]methanone;
[4-[(S)-(3-cyclopropyl-1,2,4-oxadiazol-5-yl)-(4-fluorophenyl)methyl]-1-piperidyl]−[6-(3-cyclopropyl-1,2,4-triazol-1-yl)-2-azaspiro[3.3]heptan-2-yl]methanone;
(6-((2-chloro-4-fluorophenyl)sulfonyl)-2,6-diazaspiro[3.3]heptan-2-yl)(6-(3-cyclopropyl-1H-1,2,4-triazol-1-yl)-2-azaspiro[3.3]heptan-2-yl)methanone;
2-[[2-[6-(3-cyclopropyl-1,2,4-triazol-1-yl)-2-azaspiro[3.3]heptane-2-carbonyl]-2,6-diazaspiro[3.3]heptan-6-yl]methyl]benzoic acid methyl ester;
[6-(3-cyclopropyl-1,2,4-triazol-1-yl)-2-azaspiro[3.3]heptan-2-yl]−[2-[4-(trifluoromethoxy)phenyl]sulfonyl-2,6-diazaspiro[3.3]heptan-6-yl]methanone;
[6-(3-cyclopropyl-1,2,4-triazol-1-yl)-2-azaspiro[3.3]heptan-2-yl]−[2-(2,4-difluorophenyl)sulfonyl-2,6-diazaspiro[3.3]heptan-6-yl]methanone;
[6-(3-cyclopropyl-1,2,4-triazol-1-yl)-2-azaspiro[3.3]heptan-2-yl]−[6-[[5-(trifluoromethyl)-2-pyridyl]methyl]-2,6-diazaspiro[3.3]heptan-2-yl]methanone;
[6-(3-cyclopropyl-1,2,4-triazol-1-yl)-2-azaspiro[3.3]heptan-2-yl]−[2-[3-(trifluoromethoxy)phenyl]sulfonyl-2,6-diazaspiro[3.3]heptan-6-yl]methanone;
[6-(3-cyclopropyl-1,2,4-triazol-1-yl)-2-azaspiro[3.3]heptan-2-yl]−[6-(2,4-difluorobenzyl)-2,6-diazaspiro[3.3]heptan-2-yl]methanone;
(3-((4-chloro-5-cyclopropylpyridin-3-yl)oxy)azetidin-1-yl)(6-(3-cyclopropyl-1H-1,2,4-triazol-1-yl)-2-azaspiro[3.3]heptan-2-yl)methanone;
[6-(3-cyclopropyl-1,2,4-triazol-1-yl)-2-azaspiro[3.3]heptan-2-yl]−[2-[4-fluoro-3-(trifluoromethyl)phenyl]sulfonyl-2,6-diazaspiro[3.3]heptan-6-yl]methanone;
[6-(3-cyclopropyl-1,2,4-triazol-1-yl)-2-azaspiro[3.3]heptan-2-yl]-(2-piperidinosulfonyl-2,6-diazaspiro[3.3]heptan-6-yl)methanone;
[6-(3-cyclopropyl-1,2,4-triazol-1-yl)-2-azaspiro[3.3]heptan-2-yl]−[7-(4-fluoro-2-mesyl-phenoxy)-2-azaspiro[3.5]nonan-2-yl]methanone;
[6-(3-cyclopropyl-1,2,4-triazol-1-yl)-2-azaspiro[3.3]heptan-2-yl]-(2-neopentylsulfonyl-2,6-diazaspiro[3.3]heptan-6-yl)methanone;
2-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]benzonitrile;
(6-(3-cyclopropyl-1H-1,2,4-triazol-1-yl)-2-azaspiro[3.3]heptan-2-yl)(3-(2-fluoro-4-methylphenoxy)azetidin-1-yl)methanone;
[6-(3-cyclopropyl-1,2,4-triazol-1-yl)-2-azaspiro[3.3]heptan-2-yl]−[2-(2,4,6-trifluorophenyl)sulfonyl-2,6-diazaspiro[3.3]heptan-6-yl]methanone;
[2-[(2-chloro-3-pyridyl)sulfonyl]-2,6-diazaspiro[3.3]heptan-6-yl]−[6-(3-cyclopropyl-1,2,4-triazol-1-yl)-2-azaspiro[3.3]heptan-2-yl]methanone;
[2-(cyclohexylmethylsulfonyl)-2,6-diazaspiro[3.3]heptan-6-yl]−[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[2-(3-methoxyphenyl)sulfonyl-2,6-diazaspiro[3.3]heptan-6-yl]methanone;
N-[[1-[6-(3-cyclopropyl-1,2,4-triazol-1-yl)-2-azaspiro[3.3]heptane-2-carbonyl]azetidin-3-yl]methyl]-3-(trifluoromethyl)benzenesulfonamide;
6-[6-(3-cyclopropyl-1,2,4-triazol-1-yl)-2-azaspiro[3.3]heptane-2-carbonyl]-N-[[1-(trifluoromethyl)cyclopropyl]methyl]-2,6-diazaspiro[3.3]heptane-2-sulfonamide;
(6-(3-cyclopropyl-1H-1,2,4-triazol-1-yl)-2-azaspiro[3.3]heptan-2-yl)(3-((6-(trifluoromethyl)pyridazin-3-yl)oxy)azetidin-1-yl)methanone;
(6-(3-cyclopropyl-1H-1,2,4-triazol-1-yl)-2-azaspiro[3.3]heptan-2-yl)(3-(4-((1,1,1-trifluoropropan-2-yl)oxy)-1H-pyrazol-1-yl)azetidin-1-yl)methanone;
(2-benzofurazan-4-ylsulfonyl-2,6-diazaspiro[3.3]heptan-6-yl)-[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[2-(2-methoxyphenyl)sulfonyl-2,6-diazaspiro[3.3]heptan-6-yl]methanone;
[6-(3-cyclopropyl-1,2,4-triazol-1-yl)-2-azaspiro[3.3]heptan-2-yl]−[3-[4-[1-(2H-tetrazol-5-yl)cyclopropyl]phenyl]azetidin-1-yl]methanone;
N-[[1-[6-(3-cyclopropyl-1,2,4-triazol-1-yl)-2-azaspiro[3.3]heptane-2-carbonyl]-4-piperidyl]methyl]-4-(trifluoromethyl)benzenesulfonamide;
(3-((6-chloro-5-cyclopropylpyridin-3-yl)oxy)azetidin-1-yl)(6-(3-cyclopropyl-1H-1,2,4-triazol-1-yl)-2-azaspiro[3.3]heptan-2-yl)methanone;
[6-(3-cyclopropyl-1,2,4-triazol-1-yl)-2-azaspiro[3.3]heptan-2-yl]−[2-[[6-(trifluoromethyl)-3-pyridyl]sulfonyl]-2,6-diazaspiro[3.3]heptan-6-yl]methanone;
6-[6-(3-cyclopropyl-1,2,4-triazol-1-yl)-2-azaspiro[3.3]heptane-2-carbonyl]-N-(4-fluorobenzyl)-2,6-diazaspiro[3.3]heptane-2-sulfonamide;
2-[[2-[6-(3-cyclopropyl-1,2,4-triazol-1-yl)-2-azaspiro[3.3]heptane-2-carbonyl]-2,6-diazaspiro[3.3]heptan-6-yl]methyl]benzenesulfonamide;
[6-(3-cyclopropyl-1,2,4-triazol-1-yl)-2-azaspiro[3.3]heptan-2-yl]−[6-[(3,5-difluoro-2-pyridyl)methyl]-2,6-diazaspiro[3.3]heptan-2-yl]methanone;
N-[[1-[6-(3-cyclopropyl-1,2,4-triazol-1-yl)-2-azaspiro[3.3]heptane-2-carbonyl]-4-piperidyl]methyl]-4-(trifluoromethoxy)benzenesulfonamide;
6-[6-(3-cyclopropyl-1,2,4-triazol-1-yl)-2-azaspiro[3.3]heptane-2-carbonyl]-N-[1-(trifluoromethyl)cyclopropyl]-2,6-diazaspiro[3.3]heptane-2-sulfonamide;
4-(1-(6-(3-cyclopropyl-1H-1,2,4-triazol-1-yl)-2-azaspiro[3.3]heptane-2-carbonyl)azetidin-3-yl)-1-(2,2,2-trifluoroethyl)pyridin-2(1H)-one;
[6-(3-cyclopropyl-1,2,4-triazol-1-yl)-2-azaspiro[3.3]heptan-2-yl]−[3-[[2-fluoro-4-(trifluoromethyl)benzyl]amino]azetidin-1-yl]methanone;
[6-(3-cyclopropyl-1,2,4-triazol-1-yl)-2-azaspiro[3.3]heptan-2-yl]−[2-[[4-(trifluoromethyl)-3-pyridyl]sulfonyl]-2,6-diazaspiro[3.3]heptan-6-yl]methanone;
2-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]benzoic acid methyl ester;
(2-benzylsulfonyl-2,6-diazaspiro[3.3]heptan-6-yl)-[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[3-(2-fluoro-4-mesyl-benzyl)oxyazetidin-1-yl]methanone;
[6-(3-cyclopropyl-1,2,4-triazol-1-yl)-2-azaspiro[3.3]heptan-2-yl]−[6-[[6-(trifluoromethyl)pyridazin-3-yl]amino]-2-azaspiro[3.3]heptan-2-yl]methanone;
[6-(3-cyclopropyl-1,2,4-triazol-1-yl)-2-azaspiro[3.3]heptan-2-yl]−[2-[[5-(trifluoromethyl)-3-pyridyl]sulfonyl]-2,6-diazaspiro[3.3]heptan-6-yl]methanone;
6-[6-(3-cyclopropyl-1,2,4-triazol-1-yl)-2-azaspiro[3.3]heptane-2-carbonyl]-N-(1-methylcyclopropyl)-2,6-diazaspiro[3.3]heptane-2-sulfonamide;
(6-(3-cyclopropyl-1H-1,2,4-triazol-1-yl)-2-azaspiro[3.3]heptan-2-yl)(3-((6-(trifluoromethyl)pyridin-3-yl)oxy)azetidin-1-yl)methanone;
4-chloro-N-[[1-[6-(3-cyclopropyl-1,2,4-triazol-1-yl)-2-azaspiro[3.3]heptane-2-carbonyl]-4-piperidyl]methyl]benzenesulfonamide;
[6-(3-cyclopropyl-1,2,4-triazol-1-yl)-2-azaspiro[3.3]heptan-2-yl]−[2-[2-(4-fluorophenyl)ethylsulfonyl]-2,6-diazaspiro[3.3]heptan-6-yl]methanone;
[6-(3-cyclopropyl-1,2,4-triazol-1-yl)-2-azaspiro[3.3]heptan-2-yl]−[2-(3,4-difluorophenyl)sulfonyl-2,6-diazaspiro[3.3]heptan-6-yl]methanone;
(6-(3-cyclopropyl-1H-1,2,4-triazol-1-yl)-2-azaspiro[3.3]heptan-2-yl)(3-((2-cyclopropylpyrimidin-4-yl)oxy)azetidin-1-yl)methanone;
(6-(6-(3-cyclopropyl-1H-1,2,4-triazol-1-yl)-2-azaspiro[3.3]heptane-2-carbonyl)-2,6-diazaspiro[3.3]heptan-2-yl)(4-fluoro-2-(trifluoromethyl)phenyl)methanone;
N-[[1-[6-(3-cyclopropyl-1,2,4-triazol-1-yl)-2-azaspiro[3.3]heptane-2-carbonyl]azetidin-3-yl]methyl]-4-(trifluoromethyl)benzenesulfonamide;
N-[2-[6-(3-cyclopropyl-1,2,4-triazol-1-yl)-2-azaspiro[3.3]heptane-2-carbonyl]-2-azaspiro[3.3]heptan-6-yl]-1-(trifluoromethyl)cyclopropanecarboxamide;
[2-[(4-chloro-3-pyridyl)sulfonyl]-2,6-diazaspiro[3.3]heptan-6-yl]−[6-(3-cyclopropyl-1,2,4-triazol-1-yl)-2-azaspiro[3.3]heptan-2-yl]methanone;
2-[3-[1-[6-(3-cyclopropyl-1,2,4-triazol-1-yl)-2-azaspiro[3.3]heptane-2-carbonyl]azetidin-3-yl]oxyphenyl]-2-methylpropanoic acid;
[3-(6-cyclopropylpyridazin-3-yl)oxyazetidin-1-yl]−[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[2-(3,5-dimethylisoxazol-4-yl)sulfonyl-2,6-diazaspiro[3.3]heptan-6-yl]methanone;
[6-(3-cyclopropyl-1,2,4-triazol-1-yl)-2-azaspiro[3.3]heptan-2-yl]−[2-(4-methoxyphenyl)sulfonyl-2,6-diazaspiro[3.3]heptan-6-yl]methanone;
(6-(3-cyclopropyl-1H-1,2,4-triazol-1-yl)-2-azaspiro[3.3]heptan-2-yl)(3-((2-(trifluoromethyl)pyrimidin-4-yl)oxy)azetidin-1-yl)methanone;
[6-(3-cyclopropyl-1,2,4-triazol-1-yl)-2-azaspiro[3.3]heptan-2-yl]-(2-pyrrolidinosulfonyl-2,6-diazaspiro[3.3]heptan-6-yl)methanone;
(6-(3-cyclopropyl-1H-1,2,4-triazol-1-yl)-2-azaspiro[3.3]heptan-2-yl)(3-((6-(trifluoromethyl)pyrimidin-4-yl)oxy)azetidin-1-yl)methanone;
[2-[(6-chloro-2-pyridyl)sulfonyl]-2,6-diazaspiro[3.3]heptan-6-yl]−[6-(3-cyclopropyl-1,2,4-triazol-1-yl)-2-azaspiro[3.3]heptan-2-yl]methanone;
3-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]benzonitrile;
[6-(3-cyclopropyl-1,2,4-triazol-1-yl)-2-azaspiro[3.3]heptan-2-yl]−[2-(1-methylcyclopropyl)sulfonyl-2,6-diazaspiro[3.3]heptan-6-yl]methanone;
(6-(3-cyclopropyl-1H-1,2,4-triazol-1-yl)-2-azaspiro[3.3]heptan-2-yl)(3-(5-(2,4-difluorophenyl)-4H-1,2,4-triazol-3-yl)azetidin-1-yl)methanone;
[6-(3-cyclopropyl-1,2,4-triazol-1-yl)-2-azaspiro[3.3]heptan-2-yl]−[2-(4-fluoro-2-methoxy-phenyl)sulfonyl-2,6-diazaspiro[3.3]heptan-6-yl]methanone;
(2S)-2-[1-[6-(3-cyclopropyl-1,2,4-triazol-1-yl)-2-azaspiro[3.3]heptane-2-carbonyl]-4-piperidyl]-2-(4-fluorophenyl)acetamide;
(6-(2-chloro-4-fluorobenzoyl)-2,6-diazaspiro[3.3]heptan-2-yl)(6-(3-cyclopropyl-1H-1,2,4-triazol-1-yl)-2-azaspiro[3.3]heptan-2-yl)methanone;
4-[1-[6-(3-cyclopropyl-1,2,4-triazol-1-yl)-2-azaspiro[3.3]heptane-2-carbonyl]azetidin-3-yl]benzenesulfonamide;
3-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]-4-fluoro-benzamide;
N-[4-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]phenyl]acetamide;
[2-(cyclopropylmethylsulfonyl)-2,6-diazaspiro[3.3]heptan-6-yl]−[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[2-[[2-(trifluoromethyl)-3-pyridyl]sulfonyl]-2,6-diazaspiro[3.3]heptan-6-yl]methanone;
4-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]benzamide;
3-[3-[1-[6-(3-cyclopropyl-1,2,4-triazol-1-yl)-2-azaspiro[3.3]heptane-2-carbonyl]azetidin-3-yl]oxyphenyl]-2,2-dimethylpropanoic acid;
[6-(3-cyclopropyl-1,2,4-triazol-1-yl)-2-azaspiro[3.3]heptan-2-yl]−[2-(5-methylisoxazol-4-yl)sulfonyl-2,6-diazaspiro[3.3]heptan-6-yl]methanone;
(2R)-2-[1-[6-(3-cyclopropyl-1,2,4-triazol-1-yl)-2-azaspiro[3.3]heptane-2-carbonyl]-4-piperidyl]-2-(4-fluorophenyl)acetamide;
N-[[1-[6-(3-cyclopropyl-1,2,4-triazol-1-yl)-2-azaspiro[3.3]heptane-2-carbonyl]azetidin-3-yl]methyl]-4-fluoro-2-(trifluoromethyl)benzenesulfonamide;
(6-(3-cyclopropyl-1H-1,2,4-triazol-1-yl)-2-azaspiro[3.3]heptan-2-yl)(3-((6-(trifluoromethyl)pyrazin-2-yl)oxy)azetidin-1-yl)methanone;
[6-(3-cyclopropyl-1,2,4-triazol-1-yl)-2-azaspiro[3.3]heptan-2-yl]-(2-triflyl-2,6-diazaspiro[3.3]heptan-6-yl)methanone;
2-[1-[6-(3-cyclopropyl-1,2,4-triazol-1-yl)-2-azaspiro[3.3]heptane-2-carbonyl]-4-piperidyl]-2-(4-fluorophenyl)acetamide;
(2S)-2-[4-[6-(3-cyclopropyl-1,2,4-triazol-1-yl)-2-azaspiro[3.3]heptane-2-carbonyl]piperazino]-2-(4-fluorophenyl)acetamide;
[3-[[4-(pentafluoro-16-sulfanyl)phenyl]methoxy]azetidin-1-yl]−[6-[4-(trifluoromethyl)imidazol-1-yl]-2-azaspiro[3.3]heptan-2-yl]methanone;
4-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]benzonitrile;
[6-(3-cyclopropyl-1,2,4-triazol-1-yl)-2-azaspiro[3.3]heptan-2-yl]−[2-[(2-methoxy-3-pyridyl)sulfonyl]-2,6-diazaspiro[3.3]heptan-6-yl]methanone;
[2-(2-aminopyrimidin-5-yl)sulfonyl-2,6-diazaspiro[3.3]heptan-6-yl]−[6-(3-cyclopropyl-1,2,4-triazol-1-yl)-2-azaspiro[3.3]heptan-2-yl]methanone;
2-[2-[6-(3-cyclopropyl-1,2,4-triazol-1-yl)-2-azaspiro[3.3]heptane-2-carbonyl]-2,6-diazaspiro[3.3]heptan-6-yl]-N-methyl-2-phenyl-acetamide;
1-(1-(6-(3-cyclopropyl-1H-1,2,4-triazol-1-yl)-2-azaspiro[3.3]heptane-2-carbonyl)azetidin-3-yl)-N-(2,2,2-trifluoroethyl)-1H-pyrazole-4-carboxamide;
2-[[2-[6-(3-cyclopropyl-1,2,4-triazol-1-yl)-2-azaspiro[3.3]heptane-2-carbonyl]-2,6-diazaspiro[3.3]heptan-6-yl]methyl]benzoic acid;
6-[6-(3-cyclopropyl-1,2,4-triazol-1-yl)-2-azaspiro[3.3]heptane-2-carbonyl]-N,N-dimethyl-2,6-diazaspiro[3.3]heptane-2-sulfonamide;
[6-(4-methylimidazol-1-yl)-2-azaspiro[3.3]heptan-2-yl]−[3-[[4-(pentafluoro-16-sulfanyl)phenyl]methoxy]azetidin-1-yl]methanone;
(6-(3-cyclopropyl-1H-1,2,4-triazol-1-yl)-2-azaspiro[3.3]heptan-2-yl)(3-((4-(trifluoromethyl)pyrimidin-2-yl)oxy)azetidin-1-yl)methanone;
[6-(3-cyclopropyl-1,2,4-triazol-1-yl)-2-azaspiro[3.3]heptan-2-yl]−[2-(3-pyridylsulfonyl)-2,6-diazaspiro[3.3]heptan-6-yl]methanone;
[6-(3-cyclopropyl-1,2,4-triazol-1-yl)-2-azaspiro[3.3]heptan-2-yl]-(2-morpholinosulfonyl-2,6-diazaspiro[3.3]heptan-6-yl)methanone;
3-[[1-[6-(3-cyclopropyl-1,2,4-triazol-1-yl)-2-azaspiro[3.3]heptane-2-carbonyl]-4-piperidyl]methyl]-4-fluoro-benzenesulfonamide;
[6-(3-cyclopropyl-1,2,4-triazol-1-yl)-2-azaspiro[3.3]heptan-2-yl]-(2-propylsulfonyl-2,6-diazaspiro[3.3]heptan-6-yl)methanone;
N-[1-[6-(3-cyclopropyl-1,2,4-triazol-1-yl)-2-azaspiro[3.3]heptane-2-carbonyl]-4-piperidyl]-4-(trifluoromethyl)benzenesulfonamide;
[6-(3-cyclopropyl-1,2,4-triazol-1-yl)-2-azaspiro[3.3]heptan-2-yl]−[3-[4-(trifluoromethyl)pyridazin-3-yl]oxyazetidin-1-yl]methanone;
2-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]benzamide;
4-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]benzoic acid;
N-[[1-[6-(3-cyclopropyl-1,2,4-triazol-1-yl)-2-azaspiro[3.3]heptane-2-carbonyl]-4-piperidyl]methyl]benzenesulfonamide;
(2R)-2-[4-[6-(3-cyclopropyl-1,2,4-triazol-1-yl)-2-azaspiro[3.3]heptane-2-carbonyl]piperazino]-2-(4-fluorophenyl)acetamide;
3-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]-4-fluoro-benzoic acid;
[6-[(2,4-difluorophenyl)methyl]-2-azaspiro[3.3]heptan-2-yl]−[6-(5-ethyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[2-(2,2,2-trifluoroethylsulfonyl)-2,6-diazaspiro[3.3]heptan-6-yl]methanone;
(2S)-2-[4-[6-(3-cyclopropyl-1,2,4-triazol-1-yl)-2-azaspiro[3.3]heptane-2-carbonyl]piperazino]-2-(4-fluorophenyl)-N-methyl-acetamide;
[6-(3-cyclopropyl-1,2,4-triazol-1-yl)-2-azaspiro[3.3]heptan-2-yl]−[2-(1-methylpyrazol-4-yl)sulfonyl-2,6-diazaspiro[3.3]heptan-6-yl]methanone;
2-[4-[6-(3-cyclopropyl-1,2,4-triazol-1-yl)-2-azaspiro[3.3]heptane-2-carbonyl]piperazino]-2-(4-fluorophenyl)acetamide;
(6-(3-cyclopropyl-1H-1,2,4-triazol-1-yl)-2-azaspiro[3.3]heptan-2-yl)(3-(5-(cyclopropylmethyl)-4H-1,2,4-triazol-3-yl)azetidin-1-yl)methanone;
2-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]-N-methyl-benzamide;
(2R)-2-[4-[6-(3-cyclopropyl-1,2,4-triazol-1-yl)-2-azaspiro[3.3]heptane-2-carbonyl]piperazino]-2-(4-fluorophenyl)-N-methyl-acetamide;
N-[1-[6-(3-cyclopropyl-1,2,4-triazol-1-yl)-2-azaspiro[3.3]heptane-2-carbonyl]-4-piperidyl]-4-fluoro-benzenesulfonamide;
1-[2-[6-(3-cyclopropyl-1,2,4-triazol-1-yl)-2-azaspiro[3.3]heptane-2-carbonyl]-2,6-diazaspiro[3.3]heptan-6-yl]-2,2,2-trifluoro-ethanone;
2-[[6-[6-(3-cyclopropyl-1,2,4-triazol-1-yl)-2-azaspiro[3.3]heptane-2-carbonyl]-2,6-diazaspiro[3.3]heptan-2-yl]sulfonyl]benzoic acid;
[6-(3-cyclopropyl-1,2,4-triazol-1-yl)-2-azaspiro[3.3]heptan-2-yl]−[2-(1,1-diketothietan-3-yl)sulfonyl-2,6-diazaspiro[3.3]heptan-6-yl]methanone;
2-[4-[6-(3-cyclopropyl-1,2,4-triazol-1-yl)-2-azaspiro[3.3]heptane-2-carbonyl]piperazino]-2-(4-fluorophenyl)-N-methyl-acetamide;
[3-[4-(4-chloro-2-methylsulfonyl-phenyl)phenyl]azetidin-1-yl]−[6-(triazol-2-yl)-2-azaspiro[3.3]heptan-2-yl]methanone;
[3-[4-(4-chloro-2-methylsulfonyl-phenyl)phenyl]azetidin-1-yl]−[6-(triazol-1-yl)-2-azaspiro[3.3]heptan-2-yl]methanone;
[6-[(2,4-difluorophenyl)methyl]-2-azaspiro[3.3]heptan-2-yl]−[6-(4-fluorophenyl)-2-azaspiro[3.3]heptan-2-yl]methanone;
[3-[4-(4-chloro-2-methylsulfonyl-phenyl)phenyl]azetidin-1-yl]−[6-[2-(trifluoromethyl)pyrimidin-5-yl]-2-azaspiro[3.3]heptan-2-yl]methanone;
[6-[(2,4-difluorophenyl)methyl]-2-azaspiro[3.3]heptan-2-yl]−[6-[4-(trifluoromethylsulfonyl)phenyl]-2-azaspiro[3.3]heptan-2-yl]methanone;
[6-[(2,4-difluorophenyl)methyl]-2-azaspiro[3.3]heptan-2-yl]−[6-(4-methylsulfonylphenyl)-2-azaspiro[3.3]heptan-2-yl]methanone;
[6-[(2,4-difluorophenyl)methyl]-2-azaspiro[3.3]heptan-2-yl]−[6-[2-methylsulfonyl-4-(trifluoromethyl)phenyl]-2-azaspiro[3.3]heptan-2-yl]methanone;
[6-[(5-chloro-2-pyridyl)methyl]-2-azaspiro[3.3]heptan-2-yl]−[6-(5-fluoro-3-pyridyl)-2,6-diazaspiro[3.3]heptan-2-yl]methanone;
[3-[4-[3-(2,2-dimethylpropyl)triazol-4-yl]phenyl]azetidin-1-yl]−[6-(5-fluoro-3-pyridyl)-2,6-diazaspiro[3.3]heptan-2-yl]methanone;
[3-[4-(4-chloro-2-methylsulfonyl-phenyl)phenyl]azetidin-1-yl]−[6-(5-fluoro-3-pyridyl)-2,6-diazaspiro[3.3]heptan-2-yl]methanone;
[6-[3-(1-hydroxycyclopropyl)-1,2,4-triazol-1-yl]-2-azaspiro[3.3]heptan-2-yl]−[(6S)-6-[(3, 5-difluoro-2-pyridyl)methyl]-2-azaspiro[3.4]octan-2-yl]methanone;
[6-[3-(1-hydroxycyclopropyl)-1,2,4-triazol-1-yl]-2-azaspiro[3.3]heptan-2-yl]−[(6R)-6-[(3,5-difluoro-2-pyridyl)methyl]-2-azaspiro[3.4]octan-2-yl]methanone;
[6-[[4-(trifluoromethylsulfonyl)phenyl]methyl]-2,6-diazaspiro[3.3]heptan-2-yl]−[6-[3-(trifluoromethyl)-1,2,4-triazol-1-yl]-2-azaspiro[3.3]heptan-2-yl]methanone;
[6-[[4-fluoro-2-(methylsulfonimidoyl)phenyl]methyl]-2-azaspiro[3.3]heptan-2-yl]−[6-[3-(1-hydroxycyclopropyl)-1,2,4-triazol-1-yl]-2-azaspiro[3.3]heptan-2-yl]methanone;
[3-[3-(5-cyclopropyl-3-methyl-pyrazol-1-yl)-1-bicyclo[1.1.1]pentanyl]azetidin-1-yl]−[6-[3-(1-hydroxycyclopropyl)-1,2,4-triazol-1-yl]-2-azaspiro[3.3]heptan-2-yl]methanone;
[3-[3-(5-cyclopropyl-3-methyl-pyrazol-1-yl)-1-bicyclo[1.1.1]pentanyl]azetidin-1-yl]−[6-[3-(1-hydroxycyclopropyl)-1,2,4-triazol-1-yl]-2-azaspiro[3.3]heptan-2-yl]methanone;
[3-[3-(3,5-dimethylpyrazol-1-yl)-1-bicyclo[1.1.1]pentanyl]azetidin-1-yl]−[6-[3-(1-hydroxycyclopropyl)-1,2,4-triazol-1-yl]-2-azaspiro[3.3]heptan-2-yl]methanone;
[6-(3-cyclopropyl-1,2,4-triazol-1-yl)-2-azaspiro[3.3]heptan-2-yl]−[6-(1H-pyrazolo[4,3-b]pyridin-5-ylmethyl)-2-azaspiro[3.3]heptan-2-yl]methanone;
[6-[(2,4-difluorophenyl)methyl]-2-azaspiro[3.3]heptan-2-yl]−[6-[6-(trifluoromethyl)-3-pyridyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[[3-methylsulfonyl-5-(trifluoromethyl)phenyl]methyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[(6S)-6-[[3-(trifluoromethylsulfonyl)phenyl]methyl]-2-azaspiro[3.4]octan-2-yl]methanone;
[6-[3-(1-hydroxycyclopropyl)-1,2,4-triazol-1-yl]-2-azaspiro[3.3]heptan-2-yl]−[(6R)-6-[[3-(trifluoromethylsulfonyl)phenyl]methyl]-2-azaspiro[3.4]octan-2-yl]methanone;
[6-[3-(1-hydroxycyclopropyl)-1,2,4-triazol-1-yl]-2-azaspiro[3.3]heptan-2-yl]−[(6S)-6-[[4-(trifluoromethylsulfonyl)phenyl]methyl]-2-azaspiro[3.4]octan-2-yl]methanone;
[6-[3-(1-hydroxycyclopropyl)-1,2,4-triazol-1-yl]-2-azaspiro[3.3]heptan-2-yl]−[(6R)-6-[[4-(trifluoromethylsulfonyl)phenyl]methyl]-2-azaspiro[3.4]octan-2-yl]methanone;
[6-[(4-cyclopropylsulfonylphenyl)methyl]-2-azaspiro[3.3]heptan-2-yl]−[6-[3-(1-hydroxycyclopropyl)-1,2,4-triazol-1-yl]-2-azaspiro[3.3]heptan-2-yl]methanone;
5-[[2-[6-[3-(1-hydroxycyclopropyl)-1,2,4-triazol-1-yl]-2-azaspiro[3.3]heptane-2-carbonyl]-2-azaspiro[3.3]heptan-6-yl]methyl]-2-(trifluoromethyl)benzonitrile;
[3-[4-(4-chloro-2-methylsulfonyl-phenyl)phenyl]azetidin-1-yl]−[6-(5-fluoro-3-pyridyl)-2-azaspiro[3.3]heptan-2-yl]methanone;
[6-[(5-chloro-3-fluoro-2-pyridyl)methyl]-2-azaspiro[3.3]heptan-2-yl]−[6-[3-(1-hydroxycyclopropyl)-1,2,4-triazol-1-yl]-2-azaspiro[3.3]heptan-2-yl]methanone;
4-[[2-[6-[3-(1-hydroxycyclopropyl)-1,2,4-triazol-1-yl]-2-azaspiro[3.3]heptane-2-carbonyl]-2-azaspiro[3.3]heptan-6-yl]methyl]-2-(trifluoromethyl)benzonitrile;
[6-[3-(1-hydroxycyclopropyl)-1,2,4-triazol-1-yl]-2-azaspiro[3.3]heptan-2-yl]−[7-[[4-(trifluoromethylsulfonyl)phenyl]methyl]-2,7-diazaspiro[3.4]octan-2-yl]methanone;
3-[[2-[6-[3-(1-hydroxycyclopropyl)-1,2,4-triazol-1-yl]-2-azaspiro[3.3]heptane-2-carbonyl]-2-azaspiro[3.3]heptan-6-yl]methyl]-5-(trifluoromethyl)benzonitrile;
[3-[4-(4-chloro-2-methylsulfonyl-phenyl)phenyl]azetidin-1-yl]−[6-[6-(trifluoromethyl)-3-pyridyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[3-[6-[[1-(trifluoromethyl)cyclopropyl]methylamino]-3-pyridyl]azetidin-1-yl]methanone;
[6-[3-(1-hydroxycyclopropyl)-1,2,4-triazol-1-yl]-2-azaspiro[3.3]heptan-2-yl]−[3-[6-[(3R)-3-(trifluoromethyl)pyrrolidin-1-yl]-3-pyridyl]azetidin-1-yl]methanone;
[6-[3-(1-hydroxycyclopropyl)-1,2,4-triazol-1-yl]-2-azaspiro[3.3]heptan-2-yl]−[6-[[3-(methylsulfonimidoyl)phenyl]methyl]-2-azaspiro[3.3]heptan-2-yl]methanone;
[6-[(4-dimethylphosphorylphenyl)methyl]-2-azaspiro[3.3]heptan-2-yl]−[6-[3-(trifluoromethyl)-1,2,4-triazol-1-yl]-2-azaspiro[3.3]heptan-2-yl]methanone;
[6-(3-cyclopropyl-1,2,4-triazol-1-yl)-2-azaspiro[3.3]heptan-2-yl]−[6-[(4-dimethylphosphorylphenyl)methyl]-2-azaspiro[3.3]heptan-2-yl]methanone;
[6-[(5-dimethylphosphoryl-2-pyridyl)methyl]-2-azaspiro[3.3]heptan-2-yl]−[6-[3-(trifluoromethyl)-1,2,4-triazol-1-yl]-2-azaspiro[3.3]heptan-2-yl]methanone;
[6-(3-cyclopropyl-1,2,4-triazol-1-yl)-2-azaspiro[3.3]heptan-2-yl]−[6-[(5-dimethylphosphoryl-2-pyridyl)methyl]-2-azaspiro[3.3]heptan-2-yl]methanone;
[6-[(4-dimethylphosphorylphenyl)methyl]-2,6-diazaspiro[3.3]heptan-2-yl]−[6-[3-(trifluoromethyl)-1,2,4-triazol-1-yl]-2-azaspiro[3.3]heptan-2-yl]methanone;
[6-[(2,4-difluorophenyl)methyl]-2-azaspiro[3.3]heptan-2-yl]−[6-(5-fluoro-3-pyridyl)-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[[6-(trifluoromethoxy)-3-pyridyl]methyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[3-[3-[[6-(trifluoromethyl)-3-pyridyl]methyl]-1-bicyclo[1.1.1]pentanyl]azetidin-1-yl]methanone;
[6-[3-(1-hydroxycyclopropyl)-1,2,4-triazol-1-yl]-2-azaspiro[3.3]heptan-2-yl]−[6-(1H-pyrazolo[4,3-b]pyridin-5-ylmethyl)-2-azaspiro[3.3]heptan-2-yl]methanone;
[6-(3-cyclopropyl-1,2,4-triazol-1-yl)-2-azaspiro[3.3]heptan-2-yl]−[3-[3-[(5-cyclopropyl-4H-1,2,4-triazol-3-yl)methyl]-1-bicyclo[1.1.1]pentanyl]azetidin-1-yl]methanone;
[6-[3-(1-hydroxycyclopropyl)-1,2,4-triazol-1-yl]-2-azaspiro[3.3]heptan-2-yl]−[6-[[4-methylsulfonyl-3-(trifluoromethyl)phenyl]methyl]-2-azaspiro[3.3]heptan-2-yl]methanone;
[3-[6-(4-chloro-2-methylsulfonyl-phenyl)-3-pyridyl]azetidin-1-yl]−[6-[6-(trifluoromethyl)-3-pyridyl]-2-azaspiro[3.3]heptan-2-yl]methanone;
5-[[(6S)-2-[6-[3-(1-hydroxycyclopropyl)-1,2,4-triazol-1-yl]-2-azaspiro[3.3]heptane-2-carbonyl]-2-azaspiro[3.4]octan-6-yl]oxy]-2-(trifluoromethyl)pyridine-4-carbonitrile;
[6-[(3,5-difluoro-2-pyridyl)methyl]-2-azaspiro[3.4]octan-2-yl]−[6-[3-(1-hydroxycyclopropyl)-1,2,4-triazol-1-yl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[[5-(trifluoromethyl)-2-pyridyl]methyl]-2-azaspiro[3.4]octan-2-yl]methanone;
[6-[(5-fluoro-2-pyridyl)methyl]-2-azaspiro[3.3]heptan-2-yl]−[6-[3-(1-hydroxycyclopropyl)-1,2,4-triazol-1-yl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[7-[[5-(trifluoromethyl)-2-pyridyl]methyl]-2,7-diazaspiro[3.5]nonan-2-yl]methanone;
[6-[3-(1-hydroxycyclopropyl)-1,2,4-triazol-1-yl]-2-azaspiro[3.3]heptan-2-yl]−[7-[[6-(trifluoromethyl)-3-pyridyl]methyl]-2,7-diazaspiro[3.5]nonan-2-yl]methanone;
[6-[3-(1-hydroxycyclopropyl)-1,2,4-triazol-1-yl]-2-azaspiro[3.3]heptan-2-yl]−[3-[[2-methoxy-4-(trifluoromethyl)phenyl]methylamino]azetidin-1-yl]methanone;
[6-[3-(1-hydroxycyclopropyl)-1,2,4-triazol-1-yl]-2-azaspiro[3.3]heptan-2-yl]−[3-[4-[5-[(1-methylcyclopropyl)methyl]-4H-1,2,4-triazol-3-yl]phenyl]azetidin-1-yl]methanone;
[6-[3-(1-hydroxycyclopropyl)-1,2,4-triazol-1-yl]-2-azaspiro[3.3]heptan-2-yl]−[3-[3-[[1-(trifluoromethyl)cyclopropyl]methylamino]-1-bicyclo[1.1.1]pentanyl]azetidin-1-yl]methanone;
[3-[4-(4-chloro-2-methylsulfonyl-phenyl)phenyl]azetidin-1-yl]−[6-[3-(1-hydroxycyclopropyl)-1,2,4-triazol-1-yl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[3-[6-[(3S)-3-(trifluoromethyl)pyrrolidin-1-yl]-3-pyridyl]azetidin-1-yl]methanone;
[3-[[2-fluoro-4-(trifluoromethylsulfonyl)phenyl]methoxy]azetidin-1-yl]−[6-[3-(1-hydroxycyclopropyl)-1,2,4-triazol-1-yl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[3-[[4-(trifluoromethylsulfonyl)phenyl]methoxy]azetidin-1-yl]methanone;
[6-[3-(1-hydroxycyclopropyl)-1,2,4-triazol-1-yl]-2-azaspiro[3.3]heptan-2-yl]−[6-[[4-(trifluoromethyl)-2-pyridyl]methyl]-2-azaspiro[3.3]heptan-2-yl]methanone;
N-[2-[6-[3-(1-hydroxycyclopropyl)-1,2,4-triazol-1-yl]-2-azaspiro[3.3]heptane-2-carbonyl]-2-azaspiro[3.3]heptan-6-yl]-3-(trifluoromethyl)benzenesulfonamide;
[6-[(3,5-difluoro-2-pyridyl)methyl]-2-azaspiro[3.3]heptan-2-yl]−[6-[3-(1-hydroxycyclopropyl)-1,2,4-triazol-1-yl]-2-azaspiro[3.3]heptan-2-yl]methanone;
[6-[(2-fluoro-4-methylsulfonyl-phenyl)methyl]-2-azaspiro[3.3]heptan-2-yl]−[6-[3-(1-hydroxycyclopropyl)-1,2,4-triazol-1-yl]-2-azaspiro[3.3]heptan-2-yl]methanone;
[6-[(5-chloro-2-pyridyl)methyl]-2-azaspiro[3.3]heptan-2-yl]−[6-[3-(1-hydroxycyclopropyl)-1,2,4-triazol-1-yl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[[4-(methylsulfonimidoyl)phenyl]methyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[[3-(trifluoromethylsulfonimidoyl)phenyl]methyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[[3-(trifluoromethylsulfonimidoyl)phenyl]methyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[[5-(trifluoromethyl)-2-pyridyl]methyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[[6-(trifluoromethyl)pyridazin-3-yl]methyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[[2-(trifluoromethyl)pyrimidin-5-yl]methyl]-2-azaspiro[3.3]heptan-2-yl]methanone;
[6-[(3-fluoro-5-methylsulfonyl-phenyl)methyl]-2-azaspiro[3.3]heptan-2-yl]−[6-[3-(1-hydroxycyclopropyl)-1,2,4-triazol-1-yl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[[4-(trifluoromethylsulfonyl)phenyl]methyl]-2,6-diazaspiro[3.3]heptan-2-yl]methanone;
[3-[4-(4-chloro-2-methylsulfonyl-phenyl)phenyl]azetidin-1-yl]−[6-(1,2,4-triazol-1-yl)-2-azaspiro[3.3]heptan-2-yl]methanone;
[3-[(4-dimethylphosphorylphenyl)methoxy]azetidin-1-yl]−[6-[3-(trifluoromethyl)-1,2,4-triazol-1-yl]-2-azaspiro[3.3]heptan-2-yl]methanone;
[6-(3-cyclopropyl-1,2,4-triazol-1-yl)-2-azaspiro[3.3]heptan-2-yl]−[3-[(4-dimethylphosphorylphenyl)methoxy]azetidin-1-yl]methanone;
[6-(3-cyclopropyl-1,2,4-triazol-1-yl)-2-azaspiro[3.3]heptan-2-yl]−[6-[[4-fluoro-2-(methylsulfonimidoyl)phenyl]methyl]-2-azaspiro[3.3]heptan-2-yl]methanone;
[3-[4-[3-(2,2-dimethylpropyl)triazol-4-yl]phenyl]azetidin-1-yl]−[6-(1,2,4-triazol-1-yl)-2-azaspiro[3.3]heptan-2-yl]methanone;
[3-[3-[5-[1-(trifluoromethyl)cyclopropyl]-4H-1,2,4-triazol-3-yl]-1-bicyclo[1.1.1]pentanyl]azetidin-1-yl]−[6-[3-(trifluoromethyl)-1,2,4-triazol-1-yl]-2-azaspiro[3.3]heptan-2-yl]methanone;
[6-(3-cyclopropyl-1,2,4-triazol-1-yl)-2-azaspiro[3.3]heptan-2-yl]−[3-[3-[5-[1-(trifluoromethyl)cyclopropyl]-4H-1,2,4-triazol-3-yl]-1-bicyclo[1.1.1]pentanyl]azetidin-1-yl]methanone;
[3-[3-[[[1-(trifluoromethyl)cyclopropyl]amino]methyl]-1-bicyclo[1.1.1]pentanyl]azetidin-1-yl]−[6-[3-(trifluoromethyl)-1,2,4-triazol-1-yl]-2-azaspiro[3.3]heptan-2-yl]methanone;
[3-[3-[[1-(trifluoromethyl)cyclopropyl]methylamino]-1-bicyclo[1.1.1]pentanyl]azetidin-1-yl]−[6-[3-(trifluoromethyl)-1,2,4-triazol-1-yl]-2-azaspiro[3.3]heptan-2-yl]methanone;
[3-[6-[3-(trifluoromethyl)azetidin-1-yl]-3-pyridyl]azetidin-1-yl]−[6-[3-(trifluoromethyl)-1,2,4-triazol-1-yl]-2-azaspiro[3.3]heptan-2-yl]methanone;
[3-[2-[3-(trifluoromethyl)azetidin-1-yl]pyrimidin-5-yl]azetidin-1-yl]−[6-[3-(trifluoromethyl)-1,2,4-triazol-1-yl]-2-azaspiro[3.3]heptan-2-yl]methanone;
[3-[6-[[1-(trifluoromethyl)cyclopropyl]methylamino]-3-pyridyl]azetidin-1-yl]−[6-[3-(trifluoromethyl)-1,2,4-triazol-1-yl]-2-azaspiro[3.3]heptan-2-yl]methanone;
[3-[4-[5-[(1-methylcyclopropyl)methyl]-4H-1,2,4-triazol-3-yl]phenyl]azetidin-1-yl]−[6-[3-(trifluoromethyl)-1,2,4-triazol-1-yl]-2-azaspiro[3.3]heptan-2-yl]methanone;
[3-[6-[(3S)-3-(trifluoromethyl)pyrrolidin-1-yl]-3-pyridyl]azetidin-1-yl]−[6-[3-(trifluoromethyl)-1,2,4-triazol-1-yl]-2-azaspiro[3.3]heptan-2-yl]methanone;
[3-[6-[(3R)-3-(trifluoromethyl)pyrrolidin-1-yl]-3-pyridyl]azetidin-1-yl]−[6-[3-(trifluoromethyl)-1,2,4-triazol-1-yl]-2-azaspiro[3.3]heptan-2-yl]methanone;
[3-[6-[(1,1-dioxothietan-3-yl)methylamino]-3-pyridyl]azetidin-1-yl]−[6-[3-(trifluoromethyl)-1,2,4-triazol-1-yl]-2-azaspiro[3.3]heptan-2-yl]methanone;
2-[4-[1-[6-[3-(trifluoromethyl)-1,2,4-triazol-1-yl]-2-azaspiro[3.3]heptane-2-carbonyl]azetidin-3-yl]phenyl]benzamide;
[6-[[4-(methylsulfonimidoyl)phenyl]methyl]-2-azaspiro[3.3]heptan-2-yl]−[6-[3-(trifluoromethyl)-1,2,4-triazol-1-yl]-2-azaspiro[3.3]heptan-2-yl]methanone;
[6-[[3-(methylsulfonimidoyl)phenyl]methyl]-2-azaspiro[3.3]heptan-2-yl]−[6-[3-(trifluoromethyl)-1,2,4-triazol-1-yl]-2-azaspiro[3.3]heptan-2-yl]methanone;
[6-(3-cyclopropyl-1,2,4-triazol-1-yl)-2-azaspiro[3.3]heptan-2-yl]−[3-[3-(5-cyclopropyl-4H-1,2,4-triazol-3-yl)-1-bicyclo[1.1.1]pentanyl]azetidin-1-yl]methanone;
[3-[3-(5-cyclopropyl-4H-1,2,4-triazol-3-yl)-1-bicyclo[1.1.1]pentanyl]azetidin-1-yl]−[6-[3-(trifluoromethyl)-1,2,4-triazol-1-yl]-2-azaspiro[3.3]heptan-2-yl]methanone;
[6-(3-cyclopropyl-1,2,4-triazol-1-yl)-2-azaspiro[3.3]heptan-2-yl]−[7-[[6-(trifluoromethyl)-3-pyridyl]methyl]-2,7-diazaspiro[3.4]octan-2-yl]methanone;
[6-(3-cyclopropyl-1,2,4-triazol-1-yl)-2-azaspiro[3.3]heptan-2-yl]−[3-[3-[5-(2,2,2-trifluoroethyl)-1,3,4-oxadiazol-2-yl]-1-bicyclo[1.1.1]pentanyl]azetidin-1-yl]methanone;
[3-[3-(5-cyclopropyl-1,3,4-oxadiazol-2-yl)-1-bicyclo[1.1.1]pentanyl]azetidin-1-yl]−[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[7-[[5-(trifluoromethyl)pyrazin-2-yl]methyl]-2,7-diazaspiro[3.4]octan-2-yl]methanone;
[6-(3-cyclopropyl-1,2,4-triazol-1-yl)-2-azaspiro[3.3]heptan-2-yl]−[7-[[6-(trifluoromethyl)pyridazin-3-yl]methyl]-2,7-diazaspiro[3.4]octan-2-yl]methanone;
[6-(3-cyclopropyl-1,2,4-triazol-1-yl)-2-azaspiro[3.3]heptan-2-yl]−[7-[[5-(trifluoromethyl)-2-pyridyl]methyl]-2,7-diazaspiro[3.4]octan-2-yl]methanone;
[6-(3-cyclopropyl-1,2,4-triazol-1-yl)-2-azaspiro[3.3]heptan-2-yl]−[6-[[3-(methylsulfonimidoyl)phenyl]methyl]-2-azaspiro[3.3]heptan-2-yl]methanone;
[6-(3-cyclopropyl-1,2,4-triazol-1-yl)-2-azaspiro[3.3]heptan-2-yl]−[6-[[3-(methylsulfonimidoyl)phenyl]methyl]-2-azaspiro[3.3]heptan-2-yl]methanone;
[6-(3-cyclopropyl-1,2,4-triazol-1-yl)-2-azaspiro[3.3]heptan-2-yl]−[6-[[4-(methylsulfonimidoyl)phenyl]methyl]-2-azaspiro[3.3]heptan-2-yl]methanone;
[6-(3-cyclopropyl-1,2,4-triazol-1-yl)-2-azaspiro[3.3]heptan-2-yl]−[6-[[4-(methylsulfonimidoyl)phenyl]methyl]-2-azaspiro[3.3]heptan-2-yl]methanone;
[6-(3-cyclopropyl-1,2,4-triazol-1-yl)-2-azaspiro[3.3]heptan-2-yl]−[3-[3-[[[1-(trifluoromethyl)cyclopropyl]amino]methyl]-1-bicyclo[1.1.1]pentanyl]azetidin-1-yl]methanone;
[6-(3-cyclopropyl-1,2,4-triazol-1-yl)-2-azaspiro[3.3]heptan-2-yl]−[3-[3-[[1-(trifluoromethyl)cyclopropyl]methylamino]-1-bicyclo[1.1.1]pentanyl]azetidin-1-yl]methanone;
[6-(3-cyclopropyl-1,2,4-triazol-1-yl)-2-azaspiro[3.3]heptan-2-yl]−[6-[[4-(trifluoromethylsulfonimidoyl)phenyl]methyl]-2-azaspiro[3.3]heptan-2-yl]methanone;
[6-(3-cyclopropyl-1,2,4-triazol-1-yl)-2-azaspiro[3.3]heptan-2-yl]−[6-[[3-(trifluoromethylsulfonimidoyl)phenyl]methyl]-2-azaspiro[3.3]heptan-2-yl]methanone;
[6-(3-cyclopropyl-1,2,4-triazol-1-yl)-2-azaspiro[3.3]heptan-2-yl]−[3-[4-[5-[(1-methylcyclopropyl)methyl]-4H-1,2,4-triazol-3-yl]phenyl]azetidin-1-yl]methanone;
[6-(3-cyclopropyl-1,2,4-triazol-1-yl)-2-azaspiro[3.3]heptan-2-yl]−[6-[3-(trifluoromethyl)-1,2,4-triazol-1-yl]-2-azaspiro[3.3]heptan-2-yl]methanone;
[6-(3-cyclopropyl-1,2,4-triazol-1-yl)-2-azaspiro[3.3]heptan-2-yl]−[6-(5-cyclopropyl-1,2,4-triazol-1-yl)-2-azaspiro[3.3]heptan-2-yl]methanone;
[3-[[2-methoxy-4-(trifluoromethyl)phenyl]methylamino]azetidin-1-yl]−[6-[3-(trifluoromethyl)-1,2,4-triazol-1-yl]-2-azaspiro[3.3]heptan-2-yl]methanone;
[3-[6-[3-hydroxy-3-(trifluoromethyl)azetidin-1-yl]-3-pyridyl]azetidin-1-yl]−[6-[3-(trifluoromethyl)-1,2,4-triazol-1-yl]-2-azaspiro[3.3]heptan-2-yl]methanone;
[3-[6-(3-hydroxy-3-methyl-azetidin-1-yl)-3-pyridyl]azetidin-1-yl]−[6-[3-(trifluoromethyl)-1,2,4-triazol-1-yl]-2-azaspiro[3.3]heptan-2-yl]methanone;
3-(trifluoromethyl)-N-[2-[6-[3-(trifluoromethyl)-1,2,4-triazol-1-yl]-2-azaspiro[3.3]heptane-2-carbonyl]-2-azaspiro[3.3]heptan-6-yl]benzenesulfonamide;
[6-[(4-methylsulfonylphenyl)methyl]-2-azaspiro[3.3]heptan-2-yl]−[6-[3-(trifluoromethyl)-1,2,4-triazol-1-yl]-2-azaspiro[3.3]heptan-2-yl]methanone;
[6-[(3-methylsulfonylphenyl)methyl]-2-azaspiro[3.3]heptan-2-yl]−[6-[3-(trifluoromethyl)-1,2,4-triazol-1-yl]-2-azaspiro[3.3]heptan-2-yl]methanone;
[6-[(5-methylsulfonyl-3-pyridyl)methyl]-2-azaspiro[3.3]heptan-2-yl]−[6-[3-(trifluoromethyl)-1,2,4-triazol-1-yl]-2-azaspiro[3.3]heptan-2-yl]methanone;
[6-[(5-methylsulfonyl-2-pyridyl)methyl]-2-azaspiro[3.3]heptan-2-yl]−[6-[3-(trifluoromethyl)-1,2,4-triazol-1-yl]-2-azaspiro[3.3]heptan-2-yl]methanone;
[6-[(2-fluoro-4-methylsulfonyl-phenyl)methyl]-2-azaspiro[3.3]heptan-2-yl]−[6-[3-(trifluoromethyl)-1,2,4-triazol-1-yl]-2-azaspiro[3.3]heptan-2-yl]methanone;
[6-[(3-fluoro-5-methylsulfonyl-phenyl)methyl]-2-azaspiro[3.3]heptan-2-yl]−[6-[3-(trifluoromethyl)-1,2,4-triazol-1-yl]-2-azaspiro[3.3]heptan-2-yl]methanone;
[6-(3-cyclopropyl-1,2,4-triazol-1-yl)-2-azaspiro[3.3]heptan-2-yl]−[6-[[3-(methylsulfonimidoyl)phenyl]methyl]-2-azaspiro[3.3]heptan-2-yl]methanone;
[6-(3-cyclopropyl-1,2,4-triazol-1-yl)-2-azaspiro[3.3]heptan-2-yl]−[6-[[4-(methylsulfonimidoyl)phenyl]methyl]-2-azaspiro[3.3]heptan-2-yl]methanone;
[6-(3-cyclopropyl-1,2,4-triazol-1-yl)-2-azaspiro[3.3]heptan-2-yl]−[7-[[5-(trifluoromethyl)-2-pyridyl]methyl]-2,7-diazaspiro[3.5]nonan-2-yl]methanone;
[6-(3-cyclopropyl-1,2,4-triazol-1-yl)-2-azaspiro[3.3]heptan-2-yl]−[7-[[6-(trifluoromethyl)-3-pyridyl]methyl]-2,7-diazaspiro[3.5]nonan-2-yl]methanone;
[6-(3-cyclopropyl-1,2,4-triazol-1-yl)-2-azaspiro[3.3]heptan-2-yl]−[6-[[[3-(trifluoromethyl)oxetan-3-yl]amino]methyl]-2-azaspiro[3.3]heptan-2-yl]methanone;
[6-(3-cyclopropyl-1,2,4-triazol-1-yl)-2-azaspiro[3.3]heptan-2-yl]−[3-[4-[5-[1-(trifluoromethyl)cyclopropyl]-4H-1,2,4-triazol-3-yl]phenyl]azetidin-1-yl]methanone;
[6-(3-cyclopropyl-1,2,4-triazol-1-yl)-2-azaspiro[3.3]heptan-2-yl]−[3-[4-(5-cyclopropyl-1H-1,2,4-triazol-3-yl)phenyl]azetidin-1-yl]methanone;
[6-(3-cyclopropyl-1,2,4-triazol-1-yl)-2-azaspiro[3.3]heptan-2-yl]−[6-[(3-fluoro-5-methylsulfonyl-phenyl)methyl]-2-azaspiro[3.3]heptan-2-yl]methanone;
[6-(3-cyclopropyl-1,2,4-triazol-1-yl)-2-azaspiro[3.3]heptan-2-yl]−[6-[[4-(trifluoromethylsulfonyl)phenyl]methyl]-2,6-diazaspiro[3.3]heptan-2-yl]methanone;
[6-(3-cyclopropyl-1,2,4-triazol-1-yl)-2-azaspiro[3.3]heptan-2-yl]−[3-[5-[[1-(trifluoromethyl)cyclopropyl]methylamino]pyrazin-2-yl]azetidin-1-yl]methanone;
[3-[4-(5-cyclobutyl-1H-1,2,4-triazol-3-yl)phenyl]azetidin-1-yl]−[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[6-[[6-(trifluoromethyl)pyridazin-3-yl]methyl]-2,6-diazaspiro[3.3]heptan-2-yl]methanone;
[6-(3-cyclopropyl-1,2,4-triazol-1-yl)-2-azaspiro[3.3]heptan-2-yl]−[6-[[5-(trifluoromethyl)pyrazin-2-yl]methyl]-2,6-diazaspiro[3.3]heptan-2-yl]methanone;
[6-(3-cyclopropyl-1,2,4-triazol-1-yl)-2-azaspiro[3.3]heptan-2-yl]−[2-[[3-(trifluoromethyl)-1-bicyclo[1.1.1]pentanyl]sulfonyl]-2,6-diazaspiro[3.3]heptan-6-yl]methanone;
[6-(3-cyclopropyl-1,2,4-triazol-1-yl)-2-azaspiro[3.3]heptan-2-yl]−[6-[(4-fluoro-2-methylsulfonyl-phenyl)methyl]-2-azaspiro[3.3]heptan-2-yl]methanone;
[6-(3-cyclopropyl-1,2,4-triazol-1-yl)-2-azaspiro[3.3]heptan-2-yl]−[3-[6-[(3S)-3-(trifluoromethyl)pyrrolidin-1-yl]-3-pyridyl]azetidin-1-yl]methanone;
[6-(3-cyclopropyl-1,2,4-triazol-1-yl)-2-azaspiro[3.3]heptan-2-yl]−[3-[6-[[1-(trifluoromethyl)cyclopropyl]amino]-3-pyridyl]azetidin-1-yl]methanone;
[6-(3-cyclopropyl-1,2,4-triazol-1-yl)-2-azaspiro[3.3]heptan-2-yl]−[3-[5-[[1-(trifluoromethyl)cyclopropyl]methylamino]-2-pyridyl]azetidin-1-yl]methanone;
[6-(3-cyclopropyl-1,2,4-triazol-1-yl)-2-azaspiro[3.3]heptan-2-yl]−[3-[2-[3-(trifluoromethyl)azetidin-1-yl]pyrimidin-5-yl]azetidin-1-yl]methanone;
[6-(3-cyclopropyl-1,2,4-triazol-1-yl)-2-azaspiro[3.3]heptan-2-yl]−[3-[6-[(3S)-3-(trifluoromethyl)pyrrolidin-1-yl]-3-pyridyl]azetidin-1-yl]methanone;
[6-(3-cyclopropyl-1,2,4-triazol-1-yl)-2-azaspiro[3.3]heptan-2-yl]−[3-[2-[(3S)-3-(trifluoromethyl)pyrrolidin-1-yl]pyrimidin-5-yl]azetidin-1-yl]methanone;
[6-(3-cyclopropyl-1,2,4-triazol-1-yl)-2-azaspiro[3.3]heptan-2-yl]−[3-[2-[(3R)-3-(trifluoromethyl)pyrrolidin-1-yl]pyrimidin-5-yl]azetidin-1-yl]methanone;
[6-(3-cyclopropyl-1,2,4-triazol-1-yl)-2-azaspiro[3.3]heptan-2-yl]−[6-[(2-fluoro-4-methylsulfonyl-phenyl)methyl]-2-azaspiro[3.3]heptan-2-yl]methanone;
[6-(3-cyclopropyl-1,2,4-triazol-1-yl)-2-azaspiro[3.3]heptan-2-yl]−[7-[[3-(trifluoromethyl)-1-bicyclo[1.1.1]pentanyl]sulfonyl]-2,7-diazaspiro[3.5]nonan-2-yl]methanone;
[6-(3-cyclopropyl-1,2,4-triazol-1-yl)-2-azaspiro[3.3]heptan-2-yl]−[7-[(3-methylsulfonylphenyl)methyl]-2,7-diazaspiro[3.5]nonan-2-yl]methanone;
[6-(3-cyclopropyl-1,2,4-triazol-1-yl)-2-azaspiro[3.3]heptan-2-yl]−[7-[(4-methylsulfonylphenyl)methyl]-2,7-diazaspiro[3.5]nonan-2-yl]methanone;
[6-[3-(1-hydroxycyclopropyl)-1,2,4-triazol-1-yl]-2-azaspiro[3.3]heptan-2-yl]−[6-[[5-(trifluoromethyl)pyrazin-2-yl]methyl]-2-azaspiro[3.3]heptan-2-yl]methanone;
[6-(3-cyclopropyl-1,2,4-triazol-1-yl)-2-azaspiro[3.3]heptan-2-yl]−[6-[(5-methylsulfonyl-2-pyridyl)methyl]-2-azaspiro[3.3]heptan-2-yl]methanone;
[6-(3-cyclopropyl-1,2,4-triazol-1-yl)-2-azaspiro[3.3]heptan-2-yl]−[6-[(5-methylsulfonyl-3-pyridyl)methyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[(3-methylsulfonylphenyl)methyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[6-[(4-methylsulfonylphenyl)methyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[3-[6-[[1-(trifluoromethyl)cyclopropyl]amino]-3-pyridyl]azetidin-1-yl]methanone;
(2R)-1-[4-[1-[6-(3-cyclopropyl-1,2,4-triazol-1-yl)-2-azaspiro[3.3]heptane-2-carbonyl]azetidin-3-yl]phenyl]-4,4-difluoro-piperidine-2-carboxamide;
(2R)-1-[4-[1-[6-(3-cyclopropyl-1,2,4-triazol-1-yl)-2-azaspiro[3.3]heptane-2-carbonyl]azetidin-3-yl]phenyl]-4,4-difluoro-piperidine-2-carboxamide;
[6-[3-(1-hydroxycyclopropyl)-1,2,4-triazol-1-yl]-2-azaspiro[3.3]heptan-2-yl]−[6-[[6-(trifluoromethyl)-3-pyridyl]methyl]-2-azaspiro[3.3]heptan-2-yl]methanone;
[6-[3-(1-hydroxycyclopropyl)-1,2,4-triazol-1-yl]-2-azaspiro[3.3]heptan-2-yl]−[3-[6-[3-(trifluoromethyl)azetidin-1-yl]-3-pyridyl]azetidin-1-yl]methanone;
[6-[3-(1-hydroxycyclopropyl)-1,2,4-triazol-1-yl]-2-azaspiro[3.3]heptan-2-yl]−[2-[3-(trifluoromethoxy)phenyl]sulfonyl-2,6-diazaspiro[3.3]heptan-6-yl]methanone;
[6-(3-cyclopropyl-1,2,4-triazol-1-yl)-2-azaspiro[3.3]heptan-2-yl]−[3-[[3-(trifluoromethylsulfonyl)phenyl]methoxy]azetidin-1-yl]methanone;
[6-(3-cyclopropyl-1,2,4-triazol-1-yl)-2-azaspiro[3.3]heptan-2-yl]−[3-[[4-(trifluoromethylsulfonyl)phenyl]methoxy]azetidin-1-yl]methanone;
[6-(3-cyclopropyl-1,2,4-triazol-1-yl)-2-azaspiro[3.3]heptan-2-yl]−[6-[(4-methylsulfonylphenyl)methyl]-2,6-diazaspiro[3.3]heptan-2-yl]methanone;
[6-(3-cyclopropyl-1,2,4-triazol-1-yl)-2-azaspiro[3.3]heptan-2-yl]−[6-[(3-methylsulfonylphenyl)methyl]-2,6-diazaspiro[3.3]heptan-2-yl]methanone;
[6-(3-cyclopropyl-1,2,4-triazol-1-yl)-2-azaspiro[3.3]heptan-2-yl]−[6-[(2-methylsulfonylphenyl)methyl]-2,6-diazaspiro[3.3]heptan-2-yl]methanone;
1-[4-[1-[6-(3-cyclopropyl-1,2,4-triazol-1-yl)-2-azaspiro[3.3]heptane-2-carbonyl]azetidin-3-yl]phenyl]-4,4-difluoro-piperidine-2-carboxamide;
[6-(3-cyclopropyl-1,2,4-triazol-1-yl)-2-azaspiro[3.3]heptan-2-yl]−[3-[6-(3-hydroxy-3-methyl-azetidin-1-yl)-3-pyridyl]azetidin-1-yl]methanone;
[6-(3-cyclopropyl-1,2,4-triazol-1-yl)-2-azaspiro[3.3]heptan-2-yl]−[3-[6-[(1,1-dioxothiolan-3-yl)amino]-3-pyridyl]azetidin-1-yl]methanone;
[6-(3-cyclopropyl-1,2,4-triazol-1-yl)-2-azaspiro[3.3]heptan-2-yl]−[4-(3-fluorophenoxy)-1-piperidyl]methanone;
[3-(4-cyclobutylphenyl)azetidin-1-yl]−[6-(3-cyclopropyl-1,2,4-triazol-1-yl)-2-azaspiro[3.3]heptan-2-yl]methanone;
[6-(3-cyclopropyl-1,2,4-triazol-1-yl)-2-azaspiro[3.3]heptan-2-yl]−[3-[2-(2-fluoro-6-methyl-phenyl)ethyl]azetidin-1-yl]methanone;
[6-(3-cyclopropyl-1,2,4-triazol-1-yl)-2-azaspiro[3.3]heptan-2-yl]−[3-[(E)-2-(3-fluorophenyl)vinyl]azetidin-1-yl]methanone;
bis[6-(3-cyclopropyl-1,2,4-triazol-1-yl)-2-azaspiro[3.3]heptan-2-yl]methanone;
[7-[[6-(difluoromethoxy)-3-pyridyl]methyl]-2-azaspiro[3.5]nonan-2-yl]−[6-(5-fluoro-3-pyridyl)-2-azaspiro[3.3]heptan-2-yl]methanone;
[3-[3-cyclopropyl-4-(trifluoromethyl)phenoxy]azetidin-1-yl]−[6-(5-fluoro-3-pyridyl)-2-azaspiro[3.3]heptan-2-yl]methanone;
[3-[(2-chloro-4-fluoro-phenyl)methoxy]azetidin-1-yl]−[6-(5-fluoro-3-pyridyl)-2-azaspiro[3.3]heptan-2-yl]methanone;
[3-[[2-chloro-4-(trifluoromethyl)phenyl]methylamino]azetidin-1-yl]−[6-(5-fluoro-3-pyridyl)-2-azaspiro[3.3]heptan-2-yl]methanone;
[6-(5-fluoro-3-pyridyl)-2-azaspiro[3.3]heptan-2-yl]−[6-[[6-(trifluoromethyl)-3-pyridyl]methyl]-2-azaspiro[3.3]heptan-2-yl]methanone;
[7-[(5-fluoro-2-pyridyl)methyl]-2-azaspiro[3.5]nonan-2-yl]−[6-[6-(trifluoromethyl)-3-pyridyl]-2-azaspiro[3.3]heptan-2-yl]methanone;
[7-[(5-chloro-2-pyridyl)methyl]-2-azaspiro[3.5]nonan-2-yl]−[6-[6-(trifluoromethyl)-3-pyridyl]-2-azaspiro[3.3]heptan-2-yl]methanone;
[3-[[2-methoxy-4-(trifluoromethyl)phenyl]methylamino]azetidin-1-yl]−[6-[6-(trifluoromethyl)-3-pyridyl]-2-azaspiro[3.3]heptan-2-yl]methanone;
[6-[[5-(trifluoromethyl)pyrazin-2-yl]methyl]-2-azaspiro[3.3]heptan-2-yl]−[6-[6-(trifluoromethyl)-3-pyridyl]-2-azaspiro[3.3]heptan-2-yl]methanone;
[6-[6-(trifluoromethyl)-3-pyridyl]-2-azaspiro[3.3]heptan-2-yl]−[3-[[4-(trifluoromethylsulfonyl)phenyl]methoxy]azetidin-1-yl]methanone;
[7-[[6-(difluoromethoxy)-3-pyridyl]methyl]-2-azaspiro[3.5]nonan-2-yl]−[6-[6-(trifluoromethyl)-3-pyridyl]-2-azaspiro[3.3]heptan-2-yl]methanone;
bis[6-[6-(trifluoromethyl)-3-pyridyl]-2-azaspiro[3.3]heptan-2-yl]methanone;
[3-[6-[3-(trifluoromethyl)azetidin-1-yl]-3-pyridyl]azetidin-1-yl]−[6-[6-(trifluoromethyl)-3-pyridyl]-2-azaspiro[3.3]heptan-2-yl]methanone;
[6-[6-(trifluoromethyl)-3-pyridyl]-2-azaspiro[3.3]heptan-2-yl]−[6-[[6-(trifluoromethyl)-3-pyridyl]methyl]-2-azaspiro[3.3]heptan-2-yl]methanone;
[6-[6-(trifluoromethyl)-3-pyridyl]-2-azaspiro[3.3]heptan-2-yl]−[6-[[6-(trifluoromethyl)-3-pyridyl]oxy]-2-azaspiro[3.3]heptan-2-yl]methanone;
[6-[6-(trifluoromethyl)-3-pyridyl]-2-azaspiro[3.3]heptan-2-yl]−[6-[[4-(trifluoromethylsulfonyl)phenyl]methyl]-2,6-diazaspiro[3.3]heptan-2-yl]methanone;
[3-[6-[[1-(trifluoromethyl)cyclopropyl]methylamino]-3-pyridyl]azetidin-1-yl]−[6-[6-(trifluoromethyl)-3-pyridyl]-2-azaspiro[3.3]heptan-2-yl]methanone;
[3-[3-cyclopropyl-4-(trifluoromethyl)phenoxy]azetidin-1-yl]−[6-[6-(trifluoromethyl)-3-pyridyl]-2-azaspiro[3.3]heptan-2-yl]methanone;
[6-[(3-methylsulfonylphenyl)methyl]-2-azaspiro[3.3]heptan-2-yl]−[6-[6-(trifluoromethyl)-3-pyridyl]-2-azaspiro[3.3]heptan-2-yl]methanone;
[6-[(4-methylsulfonylphenyl)methyl]-2-azaspiro[3.3]heptan-2-yl]−[6-[6-(trifluoromethyl)-3-pyridyl]-2-azaspiro[3.3]heptan-2-yl]methanone;
[3-[[2-chloro-4-(trifluoromethyl)phenyl]methylamino]azetidin-1-yl]−[6-[6-(trifluoromethyl)-3-pyridyl]-2-azaspiro[3.3]heptan-2-yl]methanone;
[6-[[4-methylsulfonyl-3-(trifluoromethyl)phenyl]methyl]-2-azaspiro[3.3]heptan-2-yl]−[6-[6-(trifluoromethyl)-3-pyridyl]-2-azaspiro[3.3]heptan-2-yl]methanone;
[3-[(2-chloro-4-fluoro-phenyl)methoxy]azetidin-1-yl]−[6-[6-(trifluoromethyl)-3-pyridyl]-2-azaspiro[3.3]heptan-2-yl]methanone;
[3-[6-(4-isopropyl-N-methyl-anilino)-3-pyridyl]azetidin-1-yl]−[6-[6-(trifluoromethyl)-3-pyridyl]-2-azaspiro[3.3]heptan-2-yl]methanone;
[6-[6-(trifluoromethyl)-3-pyridyl]-2-azaspiro[3.3]heptan-2-yl]−[6-[[6-(trifluoromethyl)-3-pyridyl]methyl]-2-azaspiro[3.4]octan-2-yl]methanone;
2-(trifluoromethyl)-5-[[2-[6-[6-(trifluoromethyl)-3-pyridyl]-2-azaspiro[3.3]heptane-2-carbonyl]-2-azaspiro[3.3]heptan-6-yl]methyl]benzonitrile;
[3-[5-(2,4-dichlorophenyl)-2-pyridyl]azetidin-1-yl]−[6-[6-(trifluoromethyl)-3-pyridyl]-2-azaspiro[3.3]heptan-2-yl]methanone;
[3-[6-(2-chloro-4-methylsulfonyl-phenyl)-3-pyridyl]azetidin-1-yl]−[6-[6-(trifluoromethyl)-3-pyridyl]-2-azaspiro[3.3]heptan-2-yl]methanone; and
[3-[5-(4-chloro-2-fluoro-phenyl)-2-pyridyl]azetidin-1-yl]−[6-[6-(trifluoromethyl)-3-pyridyl]-2-azaspiro[3.3]heptan-2-yl]methanone.

17. A pharmaceutical composition comprising a compound of formula (I) according to claim 1, or a pharmaceutically acceptable salt thereof.

18. A pharmaceutical composition comprising a compound of formula (I) according to claim 16, or a pharmaceutically acceptable salt thereof, and a therapeutically inert carrier.

19. A method of treating a disease or disorder in a human in need thereof, comprising administering to the human a compound of formula (I) according to claim 1, or a pharmaceutically acceptable salt thereof, and wherein the disease or disorder is neuroinflammation, neurodegenerative disease, pain, cancer, a mental disorder, or inflammatory bowel disease.

20. The method of claim 19, wherein said disease or disorder is selected from multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome, and visceral pain.

21. (canceled)
